(19) United States
(12) Patent Application Publication
(10) Pub. No.: US 2024/0217951 A1
(43) Pub. Date: Jul. 4, 2024

(54) PCSK9 INHIBITORS AND METHODS OF USE THEREOF

(71) Applicant: AstraZeneca AB, Södertälje (SE)

(72) Inventors: Gavin Donal O’MAHONY, Gothenburg (SE); Olaf PANKNIN, Gothenburg (SE); Andrey FROLOV, Gothenburg (SE); Anna TOMBERG, Gothenburg (SE); Mateusz Piotr PLESNIAK, Gothenburg (SE); Jens Peter BRANDT, Södertälje (SE); Jan Åke LINDBERG, Södertälje (SE); Jens Patrik JOHANSSON, Gothenburg (SE)

(21) Appl. No.: 18/470,521

(22) Filed: Sep. 20, 2023

Related U.S. Application Data
(60) Provisional application No. 63/376,791, filed on Sep. 23, 2022.
(60) Provisional application No. 63/483,797, filed on Feb. 8, 2023.
(60) Provisional application No. 63/580,507, filed on Sep. 5, 2023.

(30) Foreign Application Priority Data
Aug. 31, 2023 (PK) 581/2023

(51) Int. Cl. C07D 401/14; A61K 31/497; A61K 31/498; A61K 31/4985; A61K 31/501; A61K 45/06; C07D 471/04; C07D 487/04; C07D 491/048

(52) U.S. Cl. CPC C07D 401/14; A61K 31/497; A61K 31/498; A61K 31/4985; A61K 31/501; A61K 45/06; C07D 471/04; C07D 487/04; C07D 491/048

(57) ABSTRACT

A compound with the Formula (I):

A-B—C (I)
wherein A is of the following formula:

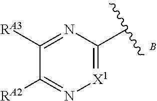

where X¹ is C—R^{A1};
B is of formula (B-1) or (B-2)

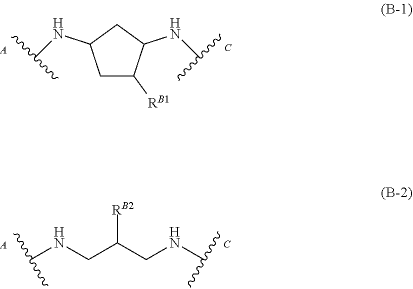

and C is selected from the group consisting of optionally substituted C₆₋₁₀ carboaryl, C₅₋₆ heteroaryl and C₅₋₁₀ heterocyclyl and their use as PCSK9 inhibitors.

### CROSS-REFERENCE TO RELATED APPLICATIONS

The application claims the benefit of priority to U.S. application No. 63/376,791, filed on 23 Sep. 2022, U.S. application No. 63/483,797, filed on 8 Feb. 2023, Pakistan application No. 581/2023, filed on 31 Aug. 2023, and U.S. application No. 63/580,507, filed on 5 Sep. 2023, which are incorporated herein by reference in their entirety.

The present disclosure relates to compounds which inhibit PCSK9 and their use in methods of treatment.

### REFERENCE TO SEQUENCE LISTING SUBMITTED ELECTRONICALLY

The content of the electronically submitted sequence listing (201191.xml; Size: 2,618 bytes; and Date of Creation: Jan. 22, 2024) submitted in this application is incorporated herein by reference in its entirety.

### BACKGROUND

PCSK9, also referred to as “proprotein convertase subtilisin/kexin 9”, is a member of the secretory proprotein convertase family and plays an important role in cholesterol metabolism. PCSK9 increases the levels of circulating LDL cholesterol (LDL-C) via the enhanced degradation of the LDLRs independently of its catalytic activity. Secreted PCSK9 binds to the Epidermal Growth Factor domain A (EGFA) of the LDL receptor (LDLR) at the cell surface and the PCSK9/LDLR complex is internalized into endosomal/lysosomal compartments. The enhanced binding affinity of PCSK9 to the LDLR at the acidic pH of late endosomes/lysosomes reduces LDLR recycling and instead targets LDLR for lysosomal degradation. Genetic association studies have demonstrated that loss-of-function mutations in PCSK9 are associated with low plasma LDL-C levels and a reduction in the incidence of adverse cardiovascular events.

For cardiovascular disease, few options exist for inhibiting PCSK9. Statins actually upregulate PCSK9 in HepG2 cells and in human primary hepatocytes through the increased expression of SREBP-2, a transcription factor that upregulates both the LDLR and PCSK9 genes. Since an elevated level of PCSK9 decreases the abundance of LDLR on the cell surface, increasing doses of statins have failed to achieve proportional LDL-C lowering effects.

Two monoclonal antibodies (mAbs) that bind selectively to extracellular PCSK9 and prevent its interaction with the LDLR, alirocumab and evolocumab, have recently received FDA approval for lowering LDL-C levels. In clinical trials, alirocumab showed an about 50% decrease in LDL levels compared to placebo (Elbitar 2016). Patients taking evolocumab showed an about 60-75% decrease in LDL levels. The potency of these drugs demonstrates the potential for inhibitors of PCSK9 to be effective treatments for those with hypercholesterolemia and other cardiovascular diseases. However, both antibody drugs require intravenous administration and can cause allergic reactions or other deleterious immune responses in the body.

Cardiovascular diseases often require management over a person's lifetime, unlike an infection that could be episodic. Thus, ease of dosing and administration become important factors for patient compliance with maintenance drug treatments. There is a need for PCSK9 inhibitors with increased efficacy and greater ease of administration, which can be achieved with small molecule PCSK9 inhibitors.

WO 2020/150473 A2 relates to heteroaryl compounds and pharmaceutical preparations thereof. It also relates to methods of treating or preventing cardiovascular diseases, and methods of treating sepsis or septic shock, using the described novel heterocyclic compounds.

WO 2020/150474 A1 relates to an inhibitor pharmacophore of PCSK9 and heteroaryl compounds that bind the PCSK9 protein.

### SUMMARY

A first aspect provides a compound with the Formula (I):

A-B—C (I)

or a pharmaceutically acceptable salt and tautomeric forms or stereoisomers thereof,

wherein A is of the following formula:

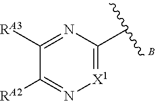

wherein the wavy line indicates the point of attachment to B;

X¹ is C—R^{A1};

R^{A1} is selected from the group consisting of:
(i) H;
(ii) halo;
(iii) CN;
(iv) C₁₋₆ hydrocarbon, optionally substituted by one or more OH, CN, C₁₋₆ acyl, C₁₋₆ alkoxy or one or more halo groups;
(v) C₁₋₆ alkoxy, optionally substituted by OH, one or more halo groups, C₁₋₆ alkyl amido;
(vi) C₁₋₆ alkylester;
(vii) C₁₋₆ alkyl acyl; and
(viii) OH;

R^{A2} is selected from the group consisting of:
(i) H;
(ii) halo;
(iii) CN;
(iv) C₁₋₆ hydrocarbon, optionally substituted by OH, CN, C₁₋₆ acyl, C₁₋₆ alkoxy or one or more halo groups;
(v) C₁₋₆ alkoxy, optionally substituted by OH, C₁₋₆ alkyl amido, or one or more halo groups;
(vi) C₁₋₆ acyl amido (wherein the acyl is optionally substituted by H or methyl);
(vii) C₁₋₆ thioalkyl optionally substituted by C₁₋₆ alkyl ester;
(viii) C₁₋₆ alkyl ester;
(ix) C₁₋₆ alkyl acyl;
(x) C₄₋₅ heterocyclyl;
(xi) C₅ heteroaryl;
(xii) C₁₋₆ alkyl amido, optionally substituted by C₁₋₃ alkyl amido, CN, OH, C₂₋₃ alkynyl, C₄₋₆ heterocyclyl, C₁₋₃ alkyl wherein the C₁₋₃ alkyl is optionally substituted with one or more halo or OH groups; and
(xiii) OH;

R^{A3} is selected from the group consisting of:
(i) H;
(ii) halo;
(iii) CN;
(iv) C₁₋₆ hydrocarbon which is optionally substituted by OH, CN, C₁₋₆ thioalkyl, C₁₋₆ alkoxy, C₁₋₆ alkyl acyl, C₁₋₆ acyloxy, carboxy, C₁₋₆ alkyl ester, C₁₋₆ alkylamino, —C(═O)NH₂, C₁₋₆ alkyl amido, C₁₋₆ alkyl acylamido, C₁₋₆ alkyl sulfinyl, C₁₋₆ alkyl sulfonyl or one or more halo groups;
(v) OH;
(vi) C₁₋₆ alkoxy, optionally substituted by OH, NH₂, C₄ heterocyclyl or one or more halo groups;
(vii) C₁₋₆ acyloxy;
(viii) C₄ heterocycyl;
(ix) NH₂;
(x) C₁₋₆ alkylamino, optionally substituted by CN, OH, Ca heterocyclyl;
(xi) C₁₋₆ dialkylamino, optionally substituted by —NH₂;
(xii) C₁₋₆ acylamido (where acyl substituent is H or Me);
(xiii) carbaimidoyl or methyl-carbaimidoyl;
(xiv) carboxyamino;
(xv) C₁₋₆ thioalkyl, optionally substituted by OH or NH₂;
(xvi) C₁₋₆ alkyl sulfinyl;
(xvii) C₁₋₆ alkyl sulfonyl, optionally substituted by one or more halo groups;
(xviii) C₁₋₆ sulfonimodyl;
(xix) C₁₋₆ alkyl phosphinyl;
(xx) carboxy;
(xxi) C(═O)NH₂
(xxii) C₁₋₆ alkyl ester;
(xxiii) C₁₋₆ alkyl acyl, optionally substituted by one or more halo groups;
(xxiv) C₁₋₆ alkyl amido;

or wherein R^{A3} and R^{A2} together with the carbon atoms to which they are bound form:
(i) an optionally substituted C₅₋₇ heterocycle ring;
(ii) an optionally substituted C₅₋₇ heteroaromatic ring;
(iii) an optionally substituted C₆ carboaromatic ring;
(iv) an optionally substituted C₅₋₇ carbocyclic ring

wherein the optional substituents are selected from C₁₋₆alkyl, halo, C₁₋₆ alkoxy, NH₂, C₁₋₆alkylamino, OH, and CN;

wherein B is of formula (B-1) or (B-2)
(i)

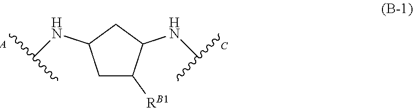

wherein the wavy line indicates the point of attachment to A and C;

R^{B1} is H, OH, ═CHCH₂—OH, O—C₁₋₄ alkyl, C₁₋₄ alkyl which C₁₋₄ alkyl is optionally substituted by OH or OMe;
(ii)

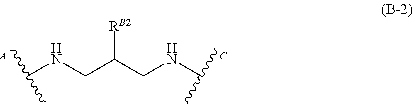

wherein the wavy line indicates the point of attachment to A and C;

R^{B2} is C₁₋₂ alkyl-OH, CH₂CONHMe or C₁₋₃ alkyl;

wherein C is selected from the group consisting of C₆₋₁₀carboaryl, C₅₋₆ heteroaryl and C₅₋₁₀ heterocyclyl, which groups are optionally substituted by:
(i) a group selected from C₆₋₁₀carboaryl, C₄₋₁₀ carbocyclyl, C₅₋₁₀ heteroaryl C₄₋₁₀ heterocyclyl, or C₅₋₁₀ bridged heterocycle, spiro C₆₋₁₂heterocyclyl or a spiro C₆₋₁₂ carbocyclyl, which are themselves optionally substituted by one or more of the following groups:
a) one or two ═O groups;
b) one or more halo groups;
c) CN, NH₂, OH;
d) one or more C₁₋₆ alkyl groups including branched and cyclic and with an optional substituent selected from OH, C₁₋₆ alkyl sulfonyl or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl optionally substituted with methyl;
i) C₄₋₁₀ carbocyclyl with an optional methyl or ═O substituent;
j) C₆₋₁₀carboaryl with optional substituents of one or more halo groups;
l) P(═O)Me₂;
m) carboxy or CH₂-carboxy; and/or
n) tetrazolyl, CH₂-tetrazolyl, 5-oxo-4H-1,2,4-oxadiazol-3-yl;

and/or
(ii) one or more groups selected from carboxy, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido, and di-C₁₋₆ alkyl sulfonamido.

A second aspect provides a pharmaceutical composition comprising the compound of the first aspect and a pharmaceutically acceptable diluent, carrier or excipient.

The third aspect provides the compound of the first aspect for use in a method of therapy. The third aspect also provides the use of a compound of the first aspect in the manufacture of a medicament for treating a cardiovascular disease. The third aspect also provides a compound of the first aspect for use in the treatment of a cardiovascular disease. The third aspect also provides a method of treating a cardiovascular disease comprising administering a therapeutically effective amount of a compound of the first aspect or a composition according to the second aspect to a patient in need thereof.

The disclosure includes the combination of the aspects and features described except where such a combination is clearly impermissible or expressly avoided.

### DETAILED DESCRIPTION

Aspects and embodiments will now be discussed. Further aspects and embodiments will be apparent to those skilled in the art. All documents mentioned in this text are incorporated herein by reference.

Compounds of Formula (I) and its use in the treatment of cardiovascular diseases are described herein. The compounds disclosed herein are PCSK9 inhibitors. The compounds may have higher inhibition of PCSK9, lower hERG activity, improved secondary pharmacology profile including GSK3p and/or other kinases, good stability, and/or improved activity in the treatment of cardiovascular diseases. The compounds may have an improved secondary pharmacology profile or an improved off-target profile.

### Definitions

### Substituents

The phrase “optionally substituted” as used herein, pertains to a parent group which may be unsubstituted or which may be substituted.

Unless otherwise specified, the term “substituted” as used herein, pertains to a parent group which bears one or more substituents. The term “substituent” is used herein in the conventional sense and refers to a chemical moiety which is covalently attached to, or if appropriate, fused to, a parent group. A wide variety of substituents are well known, and methods for their formation and introduction into a variety of parent groups are also well known.

Examples of substituents are described in more detail below.

Unless otherwise stated, halo is selected from chloro (Cl), fluoro (F), bromo (Br) and iodo (I), such as fluoro.

Cyano (nitrile, carbonitrile): —CN.

Hydroxy: —OH.

Oxo: ═O (oxygen double bonded to the rest of the molecule).

C₁₋₆ hydrocarbon: The term “C₁₋₆ hydrocarbon” as used herein pertains to a monovalent moiety obtained by removing a hydrogen atom from a carbon atom of a hydrocarbon compound having from 1 to 6 carbon atoms, which may be aliphatic or alicyclic, which may be saturated or unsaturated (e.g. partially unsaturated, fully unsaturated) and may also be branched. Thus, the term “hydrocarbon” includes the terms alkyl, alkenyl, alkynyl, cycloalkyl, etc., discussed below.

C₁₋₆ alkyl: The term “C₁₋₆ alkyl” as used herein, pertains to a monovalent moiety obtained by removing a hydrogen atom from a carbon atom of a hydrocarbon compound having from 1 to 6 carbon atoms, which are saturated and may also be branched. The term “C₁₋₄ alkyl” as used herein, pertains to a monovalent moiety obtained by removing a hydrogen atom from a carbon atom of a hydrocarbon compound having from 1 to 4 carbon atoms, which are saturated.

Examples of saturated alkyl groups include, but are not limited to, methyl (C₁), ethyl (C₂), propyl (C₃), butyl (C₄), pentyl (C₅) and hexyl (C₆).

Examples of saturated linear alkyl groups include, but are not limited to, methyl (C₁), ethyl (C₂), n-propyl (C₃), n-butyl (C₄), n-pentyl (amyl) (C₅) and n-hexyl (C₆).

Examples of saturated branched alkyl groups include iso-propyl (C₃), iso-butyl (C₄), sec-butyl (C₄), tert-butyl (C₄), iso-pentyl (C₅), and neo-pentyl (C₅).

C₂₋₆ Alkenyl: The term “C₂₋₆ alkenyl” as used herein, pertains to a hydrocarbon group having one or more carbon-carbon double bonds.

Examples of unsaturated alkenyl groups include, but are not limited to, ethenyl (vinyl, —CH═CH₂), 1-propenyl (—CH═CH—CH₃), 2-propenyl (allyl, —CH—CH═CH₂), isopropenyl (1-methylvinyl, —C(CH₃)═CH₂), butenyl (C₄), pentenyl (C₅), and hexenyl (C₆).

C₂₋₆ alkynyl: The term “C₂₋₆ alkynyl” as used herein, pertains to a hydrocarbon group having one or more carbon-carbon triple bonds.

Examples of unsaturated alkynyl groups include, but are not limited to, ethynyl (—C═CH) and 2-propynyl (propargyl, —CH₂C═CH).

C₁₋₆ alkoxy: The term C₁₋₆ alkoxy as used herein, pertains to an OR group, wherein R is an C₁₋₆ hydrocarbon group. Examples of C₁₋₆ alkoxy groups include, but are not limited to, OMe, OEt (ethoxy), —O(nPr) (n-propoxy), —O(iPr) (isopropoxy), O(nBu) (n-butoxy), O(sBu) (sec-butoxy), O(iBu) (isobutoxy), and O(tBu) (tert-butoxy).

C₁₋₆ acyloxy: The term C₁₋₆ acyloxy (reverse ester) as used herein, pertains to an —OC(═O)R, wherein R is a C₁₋₆ hydrocarbon group. Examples of acyloxy groups include, but are not limited to, —OC(═O)CH₃ (acetoxy), —OC(═O)CH₂CH₃, or —OC(═O)C(CH₃)₃.

Amino: —NR¹R², wherein R¹ and R² are independently amino substituents, for example, hydrogen, a C₁₋₆ hydrocarbon group (also referred to as C₁₋₆ alkylamino or C₁₋₆ dialkylamino), or, in the case of a “cyclic” amino group, R¹ and R², taken together with the nitrogen atom to which they are attached, form a heterocyclic ring having from 4 to 6 ring atoms. Amino groups may be primary (—NH₂), secondary (—NHR¹), or tertiary (—NHR¹R²), and in cationic form, may be quaternary (—⁺NR¹R²R³). Examples of amino groups include, but are not limited to —NH₂, —NHCH₃, —NHC(CH₃)₂, —N(CH₃)₂, —N(CH₂CH₃)₂, and —NHPh. Examples of cyclic amino groups include, but are not limited to, aziridino, azetidino, pyrrolidino, piperidino, piperazino, morpholino, and thiomorpholino.

C₁₋₆ acylamido: Acylamido (acylamino): NR¹C(═O)R², wherein R¹ is an amide substituent, for example, hydrogen or a C₁₋₆ hydrocarbon group, and R² is an acyl substituent, for example, a C₁₋₆ hydrocarbon group. Examples of acylamide groups include, but are not limited to, NHC(═O)CH₃ and NHC(═O)CH₂CH₃. In some embodiments R¹ and R² may together form a cyclic or bicyclic structure and form a cyclic acylamido groups. Examples of such groups include succinimidyl, maleimidyl, phthalimidyl, 2-oxo-3H-benzimidazol-1-yl, 3-methyl-2-oxo-benzimidazol-1-yl, 1-methyl-2-oxoimidazo[4,5-b]pyridin-3-yl, 2,5-dioxoimidazolidin-1-yl and 2,4-dioxoimidazolidin-1-yl:

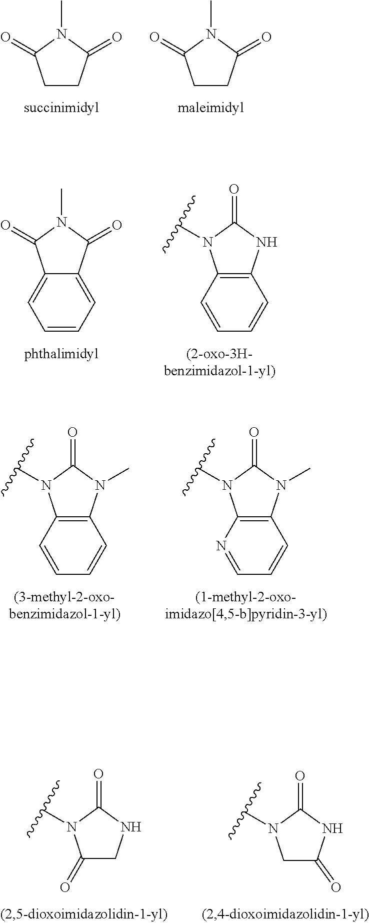

Carbaimidoyl: —C(═NH)(NH₂).

Methyl-carbaimidoyl: —C(═N—CH₃)NH₂.

Carboxyamino: —N(H)(C(═O)OH).

C₁₋₆ thioalkyl: The term C₁₋₆ thioalkyl as used herein, pertains to an —SR, wherein R is a C₁₋₆ hydrocarbon group. Examples of C₁₋₆alkylthio groups include, but are not limited to, —SCH₃ and —SCH₂CH₃.

C₁₋₆ alkyl sulfinyl: The term C₁₋₆ alkyl sulfinyl pertains to a sulfine (sulfinyl, sulfoxide) which has the structure —S(═O)R, wherein R is a C₁₋₆ hydrocarbon group. Examples of C₁₋₆ alkyl sulfinyl groups include, but are not limited to, —S(═O)CH₃ and —S(═O)CH₂CH₃.

C₁₋₆ alkyl sulfonyl: The term C₁₋₆ alkyl sulfonyl as used herein pertains to an —S(═O)₂R, group wherein R is a C₁₋₆ hydrocarbon group, including, for example, a fluorinated or perfluorinated C₁₋₆ alkyl group. Examples of C₁₋₆ alkyl sulfonyl groups include, but are not limited to, —S(═O)₂CH₃ (methanesulfonyl, mesyl), —S(═O)₂CF₃ (triflyl), —S(═O)₂CH₂CH₃ (esyl), —S(═O)₂C₄F₉ (nonaflyl) and —S(═O)₂CH₂CF₃ (tresyl).

C₁₋₆ sulfonimodyl: The term C₁₋₆ sulfonimodyl is also referred to as Sulfonamido (sulfinamoyl; sulfonic acid amide; sulfonamide) and has the structure —S(═O)₂NR¹R², wherein R¹ and R² are independently amino substituents, as defined for amino groups. Examples of sulfonamido groups include, but are not limited to, —S(═O)₂NH₂, —S(═O)₂NH(CH₃), —S(═O)₂N(CH₃)₂, —S(═O)₂NH(CH₂CH₃) and —S(═O)₂N(CH₂CH₃)₂.

C₁₋₆ alkyl phosphinyl: The term C₁₋₆ alkyl phosphinyl (phosphine oxide) has the structure —P(═O)R₂, wherein each R is independently a C₁₋₆ hydrocarbon group. Examples of C₁₋₆ alkyl phosphinyl groups include, but are not limited to, P(═O)(CH₃)₂, P(═O)(CH₂CH₃)₂ and P(═O)(tBu)₂. Wherein each R group can be the same or different groups.

Carboxy (carboxylic acid): —C(═O)OH.

C₁₋₆ alkyl ester: The term C₁₋₆ alkyl ester (carboxylate, carboxylic acid ester, oxycarbonyl) has the structure —C(═O)OR, wherein R is a C₁₋₆ hydrocarbon group. Examples of ester groups include, but are not limited to, —C(═O)OCH₃, —C(═O)OCH₂CH₃ and —C(═O)OC(CH₃)₃.

C₁₋₆ alkyl acyl: The term C₁₋₆ alkyl acyl also known as Acyl (keto) has the structure —C(═O)R, wherein R is a C₁₋₆ hydrocarbon group. Examples of C₁₋₆ alkyl acyl groups include, but are not limited to, —C(═O)CH₃ (acetyl), —C(═O)CH₂CH₃ (propionyl) or —C(═O)C(CH₃)₃(t-butyryl).

C₁₋₆ alkyl amido: The term C₁₋₆ alkyl amido (also referred to as carbamoyl, carbamyl, aminocarbonyl, carboxamide) has the structure —C(═O)NR¹R², wherein R¹ and R² are independently amino substituents, as defined for amino groups for example, hydrogen, a C₁₋₆ hydrocarbon group (also referred to as C₁₋₆ alkylamido or C₁₋₆ dialkylamido), or, in the case of a “cyclic” amido group, R¹ and R², taken together with the nitrogen atom to which they are attached, form a heterocyclic ring having from 4 to 6 ring atoms. Examples of amido groups include, but are not limited to, —C(═O)NH₂, —C(═O)NHCH₃, —C(═O)N(CH₃)₂, —C(═O)NHCH₂CH₃, and —C(═O)N(CH₂CH₃)₂, as well as amido groups in which R¹ and R², together with the nitrogen atom to which they are attached, form a heterocyclic structure as in, for example, piperidinocarbonyl, morpholinocarbonyl, thiomorpholinocarbonyl, and piperazinocarbonyl.

C₃₋₁₂ cycloalkyl: The term “C₃₋₁₂ cycloalkyl” as used herein, pertains to an alkyl group which is also a cyclyl group; that is, a monovalent moiety obtained by removing a hydrogen atom from an alicyclic ring atom of a cyclic hydrocarbon (carbocyclic) compound, which moiety has from 3 to 7 carbon atoms, including from 3 to 7 ring atoms. The carbocyclic ring may be saturated or unsaturated and may be bridged or unbridged. The ring may be a fused ring or a single ring.

Examples of cycloalkyl groups include, but are not limited to, those derived from:
saturated monocyclic hydrocarbon compounds:

cyclopropane (C₃), cyclobutane (C₄), cyclopentane (C₅), cyclohexane (C₆), cycloheptane (C₇), methylcyclopropane (C₄), dimethylcyclopropane (C₅), methylcyclobutane (C₅), dimethylcyclobutane (C₆), methylcyclopentane (C₆), dimethylcyclopentane (C₇) and methylcyclohexane (C₇);
unsaturated monocyclic hydrocarbon compounds:

cyclopropene (C₃), cyclobutene (C₄), cyclopentene (C₅), cyclohexene (C₆), methylcyclopropene (C₄), dimethylcyclopropene (C₅), methylcyclobutene (C₅), dimethylcyclobutene (C₆), methylcyclopentene (C₆), dimethylcyclopentene (C₇) and methylcyclohexene (C₇); and
saturated polycyclic hydrocarbon compounds:

norcarane (C₇), norpinane (C₇), norbornane (C₇).

C₃₋₁₀ heterocyclyl: The term “C₃₋₁₀ heterocyclyl” as used herein, pertains to a monovalent moiety obtained by removing a hydrogen atom from a ring atom of a heterocyclic compound, which moiety has from 3 to 10 ring atoms, of which from 1 to 5 are ring heteroatoms. In certain embodiments, each ring has from 3 to 7 ring atoms, of which from 1 to 4 are ring heteroatoms. The ring may be saturated or unsaturated, and may be bridged or unbridged. The ring may be a fused ring or a single ring. For the avoidance of doubt, substituents on the heterocycloalkyl ring may be linked via either a carbon atom or a heteroatom.

In this context the term ‘heteroatom’ means O, S, N, Si or B (Boron).

In this context, the prefixes (e.g. C₃₋₁₀ C₃₋₇, C₅₋₆, etc.) denote the number of ring atoms, or range of number of ring atoms, whether carbon atoms or heteroatoms. For example, the term “C₅₋₆heterocyclyl”, as used herein, pertains to a heterocyclyl group having 5 or 6 ring atoms.

Examples of monocyclic heterocyclyl groups include, but are not limited to, those derived from:

N₁: aziridine (C₃), azetidine (C₄), pyrrolidine (tetrahydropyrrole) (C₅), pyrroline (e.g., 3-pyrroline, 2,5-dihydropyrrole) (C₅), 2H-pyrrole or 3H-pyrrole (isopyrrole, isoazole) (C₅), piperidine (C₆), dihydropyridine (C₆), tetrahydropyridine (C₆), azepine (C₇);

O₁: oxirane (C₃), oxetane (C₄), oxolane (tetrahydrofuran) (C₅), oxole (dihydrofuran) (C₅), oxane (tetrahydropyran) (C₆), dihydropyran (C₆), pyran (C₆), oxepin (C₇);

S₁: thiirane (C₃), thietane (C₄), thiolane (tetrahydrothiophene) (C₅), thiane (tetrahydrothiopyran) (C₆), thiepane (C₇);

O₂: dioxolane (C₅), dioxane (C₆), and dioxepane (C₇);

O₃: trioxane (C₆);

N₂: imidazolidine (C₅), pyrazolidine (diazolidine) (C₅), imidazoline (C₅), pyrazoline (dihydropyrazole) (C₅), piperazine (C₆);

N₁O₁: tetrahydrooxazole (C₅), dihydrooxazole (C₅), tetrahydroisoxazole (C₅), dihydroisoxazole (C₅), morpholine (C₆), tetrahydrooxazine (C₆), dihydrooxazine (C₆), oxazine (C₆);

N₁S₁: thiazoline (C₅), thiazolidine (C₅), thiomorpholine (C₆);

N₂O₁: oxadiazine (C₆);

O₁S₁: oxathiole (C₅) and oxathiane (thioxane) (C₆); and,

N₁O₁S₁: oxathiazine (C₆).

Examples of bicyclic heterocyclyl groups include, but are not limited to those derived from:

| Compound | Structure |
|---|---|
| 7-azabicyclo[4.2.0]octane (N₁)C₈ | 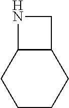 |
| 3-azabicyclo[3.1.0]hexane (N₁)C₆ | 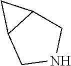 |
| 6-azabicyclo[3.2.0]heptane (N₁)C₇ | 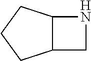 |
| 2,3,3a,4,5,6,7,7a- octahydrofuro[2,3- c]pyridine (N₁O₁)C₉ | 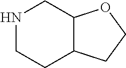 |

C₆₋₁₀ carboaryl: The term “C₆₋₁₀ carboaryl”, as used herein, pertains to a monovalent moiety obtained by removing a hydrogen atom from an aromatic ring atom of an aromatic compound, which moiety has from 6 to 10 ring atoms and the ring atoms are all carbon atoms, as in “carboaryl groups”. The ring may be a fused ring or a single ring. Examples of carboaryl groups include, but are not limited to, those derived from benzene (i.e. phenyl) (C₆), naphthalene (C₁₀) and azulene (C₁₀).

In this context, the prefixes (e.g. C₅₋₇, C₅₋₆, C₅₋₁₀, etc.) denote the number of ring atoms, or range of number of ring atoms. For example, the term “C₅₋₆ aryl” as used herein, pertains to an aryl group having 5 or 6 ring atoms.

Examples of carboaryl groups which comprise fused rings, at least one of which is an aromatic ring, include, but are not limited to, groups derived from indane (e.g. 2,3-dihydro-1H-indene) (C₉), indene (C₉), isoindene (C₉) and tetraline (1,2,3,4-tetrahydronaphthalene) (C₁₀).

C₅₋₁₀ heteroaryl: The term “C₅₋₁₀ heteroaryl”, as used herein, pertains to a monovalent moiety obtained by removing a hydrogen atom from an aromatic ring atom of an aromatic compound, which moiety has from 5 to 10 ring atoms of which from 1 to 5 are ring heteroatoms. In certain embodiments, each ring has from 5 to 7 ring atoms, of which from 1 to 4 are ring heteroatoms. For the avoidance of doubt, substituents on the heteroaryl ring may be linked via either a carbon atom or a heteroatom. The ring may be a fused ring or a single ring.

In this context the term ‘heteroatom’ means O, S, N, Si or B (Boron).

Examples of monocyclic heteroaryl groups include, but are not limited to, those derived from:

N₁: pyrrole (azole) (C₅), pyridine (azine) (C₆);

O₁n: furan (oxole) (C₅);

S₁: thiophene (thiole) (C₅);

N₁O₁: oxazole (C₅), isoxazole (C₅), isoxazine (C₆);

N₂O₁: oxadiazole (furazan) (C₅);

N₃O₁: oxatriazole (C₅);

N₁S₁: thiazole (C₅), isothiazole (C₅);

N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆);

N₃: triazole (C₁), triazine (C₆); and,

N₄: tetrazole (C₅).

Examples of heteroaryl which comprise fused rings, include, but are not limited to C₉ (with 2 fused rings) derived from:

| Compound | Structure |
|---|---|
| benzofuran (O₁) | 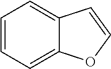 |
| isobenzofuran (O₁) | 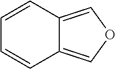 |
| indole (N₁) | 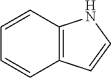 |
| isoindole (N₁) | 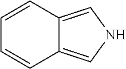 |
| indolizine (N₁) | 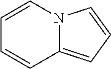 |
| indoline (N₁) | 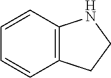 |
| isoindoline (N₁) | 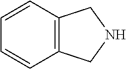 |
| purine (N₄)(e.g., adenine, guanine) | 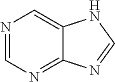 |
| | 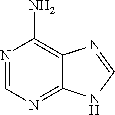 |
| | 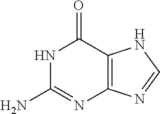 [SMILES: Nc1nc2nc[nH]c2c(=O)[nH]1] |
| benzimidazole (N₂), | 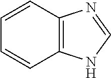 |
| indazole (N₂) | 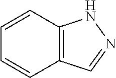 |
| benzoxazole (N₁O₁) | 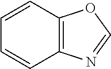 |
| benzisoxazole (N₁O₁) | 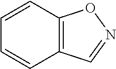 |
| benzodioxole O₂) | 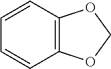 |
| | 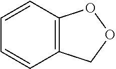 |
| benzofurazan (N₂O₁) | 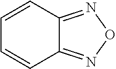 |
| benzotriazole (N₃) | 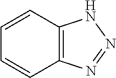 |
| benzothiofuran (S₁) | 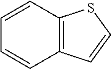 |
| benzothiazole (N₁S₁) | 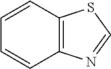 |
| benzothiadiazole (N₂S) | 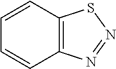 |
| 3H-imidazo[4,5- b]pyridine (N₃) | 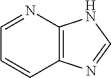 |
| 5H-pyrrolo[2,3- b]pyrazine (N₃) | 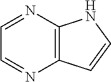 |
| 1H-pyrazolo[3,4- b]pyrazine (N₄) | 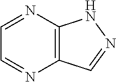 |

Examples of heteroaryl which comprise fused rings, include, but are not limited to C₁₀ (with 2 fused rings) derived from:

| Compound | Structure | |
|---|---|---|
| 2H-chromene (O₁) | 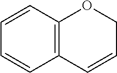 | |
| 1H- isochromene (O₁) | 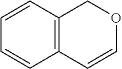 | |
| chromane (O₁) | 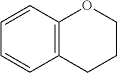 | |
| isochromane (O₁) | 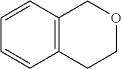 | |
| 3,4-dihydro- benzo[c][1,2]dioxine (O₂) | 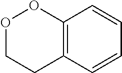 | |
| quinoline (N₁) | 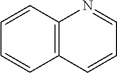 | |
| isoquinoline (N₁) | 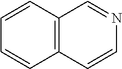 | |
| quinolizine (N₁) | 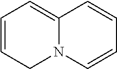 | |
| benzoxazine (N₁O₁) | 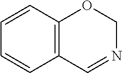 | |
| | 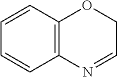 | |
| | 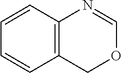 | |
| | And isomers thereof | |
| benzodiazine (N₂) | 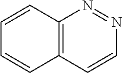 | |
| pteridine (N₄) | 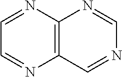 | |
| quinoxaline (N₂) | 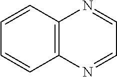 | |
| quinazoline (N₂) | 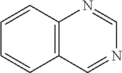 | |
| cinnoline (N₂) | 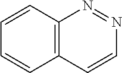 | |
| phthalazine (N₂) | 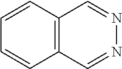 | |
| naphthyridine (N₂) | 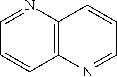 | 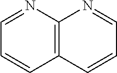 |
| | 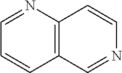 | 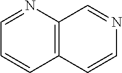 |

And isomers thereof

Spiro C₆₋₁₂ carbocyclyl: The term Spiro C₆₋₁₂carbocyclyl as used herein pertains to a moiety that has at least two molecular rings with only one common atom. The simplest spiro compounds are bicyclic (having just two rings), or have a bicyclic portion as part of the larger ring system, in either case with the two rings connected through the defining single common atom. Spiro C₆₋₁₂carbocyclyl pertains to a cyclyl group; that is, a monovalent moiety obtained by removing a hydrogen atom from an alicyclic ring atom of a cyclic hydrocarbon (carbocyclic) compound, which moiety has from 6 to 12 carbon atoms, including from 3 to 7 ring atoms wherein the rings share a common atom.

Spiro C₆₋₁₂ heterocyclyl: The term Spiro C₆₋₁₂heterocyclyl as used herein pertains to a moiety that has at least two molecular rings with only one common atom. The simplest spiro compounds are bicyclic (having just two rings), or have a bicyclic portion as part of the larger ring system, in either case with the two rings connected through the defining single common atom. The spiro C₆₋₁₂heterocyclyl moiety pertains to a monovalent moiety obtained by removing a hydrogen atom from a ring atom of a heterocyclic compound, which moiety has from 8 to 12 ring atoms of which from 1 to 3 are ring heteroatoms wherein the rings share a common atom. In certain embodiments, each ring has from 9 to 11 ring atoms, of which from 1 to 2 are ring heteroatoms. For the avoidance of doubt, substituents on the heteroaryl ring may be linked via either a carbon atom or a heteroatom.

For the avoidance of doubt, where multiple substituents are independently selected from a given group, the selected substituents may comprise the same substituents or different substituents from within the given group.

Pharmaceutically Acceptable Salt

The term “pharmaceutically acceptable” is used to specify that an object (for example a salt, dosage form or excipient) is suitable for use in patients. An example list of pharmaceutically acceptable salts can be found in the Handbook of Pharmaceutical Salts: Properties, Selection and Use, P. H. Stahl and C. G. Wermuth, editors, Weinheim/Zürich: Wiley-VCH/VHCA, 2002. A suitable pharmaceutically acceptable salt of a compound of Formula (I) is, for example, an acid-addition salt. An acid addition salt of a compound of Formula (I) may be formed by bringing the compound into contact with a suitable inorganic or organic acid under conditions known to the skilled person. An acid addition salt may for example be formed using an inorganic acid selected from the group consisting of hydrochloric acid, hydrobromic acid, sulphuric acid and phosphoric acid. An acid addition salt may also be formed using an organic acid selected from the group consisting of trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid and para-toluenesulfonic acid.

Therefore, in one embodiment there is provided a compound of Formula (I) or a pharmaceutically acceptable salt thereof, where the pharmaceutically acceptable salt is a hydrochloric acid, hydrobromic acid, sulphuric acid, phosphoric acid, trifluoroacetic acid, citric acid, maleic acid, oxalic acid, acetic acid, formic acid, benzoic acid, fumaric acid, succinic acid, tartaric acid, lactic acid, pyruvic acid, methanesulfonic acid, benzenesulfonic acid or para-toluenesulfonic acid salt. In one embodiment there is provided a compound of Formula (I) or a pharmaceutically acceptable salt thereof, where the pharmaceutically acceptable salt is a methanesulfonic acid salt. In one embodiment there is provided a compound of Formula (I) or a pharmaceutically acceptable salt thereof, where the pharmaceutically acceptable salt is a mono-methanesulfonic acid salt, i.e. the stoichiometry of the compound of the compound of Formula (I) to methanesulfonic acid is 1:1.

### Other Forms

Compounds and salts described in this specification may exist in solvated forms and unsolvated forms. For example, a solvated form may be a hydrated form, such as a hemihydrate, a monohydrate, a dihydrate, a trihydrate or an alternative quantity thereof. The compounds of Formula (I) encompass all such solvated and unsolvated forms of compounds of Formula (I), particularly to the extent that such forms possess PCSK9 kinase inhibitory activity, as for example measured using the tests described herein.

Compounds and salts described in this specification include one or more chiral (i.e. asymmetric) centres. To the extent a structure or chemical name in this specification does not indicate the chirality, the structure or name is intended to encompass any single stereoisomer (i.e. any single chiral isomer) corresponding to that structure or name, as well as any mixture of stereoisomers (e.g. a racemate). In some embodiments, a single stereoisomer is obtained by isolating it from a mixture of isomers (e.g. a racemate) using, for example, chiral chromatographic separation. In other embodiments, a single stereoisomer is obtained through direct synthesis from, for example, a chiral starting material.

A particular enantiomer of a compound described herein may be more active than other enantiomers of the same compound.

According to one embodiment there is provided a compound of Formula (I), or a pharmaceutically acceptable salt thereof, which is a single enantiomer being in an enantiomeric excess (% ee) of ≥95, ≥98% or ≥99%. Conveniently, the single enantiomer is present in an enantiomeric excess (% ee) of ≥99%.

According to another embodiment there is provided a pharmaceutical composition, which comprises a compound of Formula (I), which is a single enantiomer being in an enantiomeric excess (% ee) of ≥95, ≥98% or ≥99% or a pharmaceutically acceptable salt thereof, in association with one or more pharmaceutically acceptable excipients. Conveniently, the single enantiomer is present in an enantiomeric excess (% ee) of ≥99%.

### Isotopes

Atoms of the compounds and salts described in this specification may exist as their isotopes. The compound of Formula (I) encompasses all compounds of Formula (I) where an atom is replaced by one or more of its isotopes (for example a compound of Formula (I) where one or more carbon atom is an ¹¹C or ¹³C carbon isotope, or where one or more hydrogen atoms is a ²H or ³H isotope).

### Tautomers

Compounds and salts described in this specification may exist as a mixture of tautomers. “Tautomers” are structural isomers that exist in equilibrium resulting from the migration of a hydrogen atom. The compound of Formula (I) includes all tautomers of compounds of Formula (I) particularly to the extent that such tautomers possess PCSK9 inhibitory activity.

For example, tautomeric forms of some of the exemplified compounds wherein R^{A1} is OH can be shown as follows.

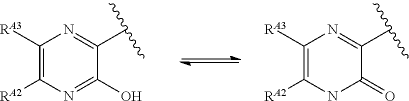

### Crystalline Forms

Compounds and salts described in this specification may be crystalline and may exhibit one or more crystalline forms. The compound of Formula (I) encompasses any crystalline or amorphous form of a compound of Formula (I), or mixture of such forms, which possesses PCSK9 inhibitory activity.

It is generally known that crystalline materials may be characterised using conventional techniques such as X-Ray Powder Diffraction (XRPD), Differential Scanning Calorimetry (DSC), Thermal Gravimetric Analysis (TGA), Diffuse Reflectance Infrared Fourier Transform (DRIFT) spectroscopy, Near Infrared (NIR) spectroscopy, solution and/or solid state nuclear magnetic resonance spectroscopy. The water content of crystalline materials may be determined by Karl Fischer analysis.

### Therapy, Prophylaxis and Related Terms

The term “therapy” is intended to have its normal meaning of dealing with a disease in order to entirely or partially relieve one, some or all of its symptoms, or to correct or compensate for the underlying pathology. The term “therapy” also includes “prophylaxis” unless there are specific indications to the contrary. The terms “therapeutic” and “therapeutically” should be interpreted in a corresponding manner.

The term “prophylaxis” is intended to have its normal meaning and includes primary prophylaxis to prevent the development of the disease and secondary prophylaxis whereby the disease has already developed and the patient is temporarily or permanently protected against exacerbation or worsening of the disease or the development of new symptoms associated with the disease.

The term “treatment” is used synonymously with “therapy”. Similarly the term “treat” can be regarded as “applying therapy” where “therapy” is as defined herein.

The term “subject” to which administration is contemplated includes, but is not limited to, humans (i.e., a male or female of any age group, e.g., a paediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

An “effective amount”, as used herein, refers to an amount that is sufficient to achieve a desired biological effect. A “therapeutically effective amount”, as used herein refers to an amount that is sufficient to achieve a desired therapeutic effect. For example, a therapeutically effective amount can refer to an amount that is sufficient to improve at least one sign or symptom of the disease to be treated.

### Pharmaceutical Compositions

The compounds of Formula (I), and pharmaceutically acceptable salts thereof, may be administered as pharmaceutical compositions, comprising one or more pharmaceutically acceptable excipients.

Therefore, in one embodiment there is provided a pharmaceutical composition comprising a compound of Formula (I), or a pharmaceutically acceptable salt thereof, and at least one pharmaceutically acceptable excipient.

The excipient(s) selected for inclusion in a particular composition will depend on factors such as the mode of administration and the form of the composition provided. Suitable pharmaceutically acceptable excipients are well known to persons skilled in the art and are described, for example, in the Handbook of Pharmaceutical Excipients, Sixth edition, Pharmaceutical Press, edited by Rowe, Ray C; Sheskey, Paul J; Quinn, Marian. Pharmaceutically acceptable excipients may function as, for example, adjuvants, diluents, carriers, stabilisers, flavourings, colorants, fillers, binders, disintegrants, lubricants, glidants, thickening agents and coating agents. As persons skilled in the art will appreciate, certain pharmaceutically acceptable excipients may serve more than one function and may serve alternative functions depending on how much of the excipient is present in the composition and what other excipients are present in the composition.

The pharmaceutical compositions may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous or intramuscular dosing), or as a suppository for rectal dosing. The compositions may be obtained by conventional procedures well known in the art. Compositions intended for oral use may contain additional components, for example, one or more colouring, sweetening, flavouring and/or preservative agents.

Suitable daily doses of the compounds disclosed herein, or a pharmaceutically acceptable salt thereof, in therapeutic treatment of humans are about 0.0001-100 mg/kg body weight. Pharmaceutical formulations as described herein may be formulated by methods known to those skilled in the art to provide doses of the active compound in the range of 0.1 mg to 1000 mg. The daily dose will necessarily be varied depending upon the host treated, the particular route of administration, any therapies being co-administered, and the severity of the illness being treated. Accordingly, the practitioner who is treating any particular patient may determine the optimum dosage.

The pharmaceutical compositions described herein comprise compounds of Formula (I), or a pharmaceutically acceptable salt thereof, and are therefore expected to be useful in therapy.

As such, in one embodiment there is provided a pharmaceutical composition for use in therapy, comprising a compound of Formula (I), or a pharmaceutically acceptable salt thereof, and at least one pharmaceutically acceptable excipient.

In one embodiment there is provided a pharmaceutical composition for use in the treatment of a disease in which inhibition of PCSK9 is beneficial, comprising a compound of Formula (I), or a pharmaceutically acceptable salt thereof, and at least one pharmaceutically acceptable excipient. In one embodiment there is provided a pharmaceutical composition for use in the treatment of a cardiovascular disease, comprising a compound of Formula (I), or a pharmaceutically acceptable salt thereof, and at least one pharmaceutically acceptable excipient. In one embodiment there is provided a pharmaceutical composition for use in the treatment of a cardiovascular disease in which inhibition of PCSK9 is beneficial, comprising a compound of Formula (I), or a pharmaceutically acceptable salt thereof, and at least one pharmaceutically acceptable excipient.

Methods of Use

The compounds described herein may be used in a method of therapy. Also provided is a method of treatment, comprising administering to a subject in need of treatment a therapeutically effective amount of a compound of formula I. The term “therapeutically effective amount” is an amount sufficient to show benefit to a patient. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors.

A compound may be administered alone or in combination with other treatments, either simultaneously or sequentially dependent upon the condition to be treated.

In one embodiment there is provided a compound of Formula (I), or a pharmaceutically acceptable salt thereof, or a pharmaceutical composition comprising the compound of Formula (I) for use in therapy. In one embodiment there is provided the use of the compound of Formula (I), or a pharmaceutically acceptable salt thereof, or a pharmaceutical composition comprising the compound of Formula (I) for the manufacture of a medicament. In another embodiment there is provided a method of treatment comprising administering to a subject the compound of Formula (I), or a pharmaceutically acceptable salt thereof, or a pharmaceutical composition comprising the compound of Formula (I).

The compounds described herein are PCSK9 inhibitors. The PCSK9 gene was identified using genetic mapping techniques on DNA from subjects with autosomal dominant hypercholesterolemia (Abifadel 2003). The encoded protein is a serine protease that is mostly expressed in the liver, gut, kidney, and nervous system and circulates in plasma. While not wishing to be bound by any particular theory, studies on mutations in the gene indicated that its putative role was in reducing LDLRs at the cell surface independently of its catalytic activity. (Abifadel 2010). Binding of PCSK9 to the LDLR results in their lysosomal degradation. This enhanced LDLR degradation results in increases in the amount of circulating low-density lipoprotein (LDL). PCSK9 is upregulated by statins, SREBP-1a and SREBP-2, LXR agonist, and insulin, but downregulated by dietary cholesterol, glucagon, ethinylestradiol, chenodeoxycholic acid and the bile acid-activated farnesoid X receptor (FXR) (Maxwell 2003; Persson 2009; Langhi 2008). Since an elevated level of PCSK9 decreases the abundance of LDLR on the cell surface, increasing doses of statins fail to achieve proportional LDL-C lowering results. Thus, disclosed herein are methods for treating a wide range of cardiovascular diseases and conditions that benefit from inhibiting PCSK9 thereby lowering LDL-C.

In certain embodiments, the method of inhibiting PCSK9 occurs in a subject in need thereof, thereby treating a disease or disorder mediated by PCSK9. Also, disclosed herein are methods of treating or preventing a disease or a disorder mediated by PCSK9 comprising administering a compound of Formula (I) or a pharmaceutically acceptable salt thereof. In certain embodiments, disclosed herein are methods of treating a disease or a disorder mediated by PCSK9 comprising administering a compound of Formula (I) or a pharmaceutically acceptable salt thereof. In certain embodiments, disclosed herein are methods of preventing a disease or a disorder mediated by PCSK9 comprising administering a compound of Formula (I) or a pharmaceutically acceptable salt thereof. The prevention of cardiovascular events through the inhibition of PCSK9 has been described, e.g., in Robinson 2015.

In some embodiments there is provided a method of treating a cardiovascular disease comprising administering to a subject a compound of Formula (I), or a pharmaceutical composition comprising a compound of Formula (I). In some embodiments there is provided a compound of Formula (I), or a pharmaceutically acceptable salt thereof, or a pharmaceutical composition comprising a compound of Formula (I) for use in the treatment of a cardiovascular disease. In some embodiments there is provided a compound of Formula (I), or a pharmaceutically acceptable salt thereof, or a pharmaceutical composition comprising a compound of Formula (I) for the manufacture of a medicament for the treatment of a cardiovascular disease.

Exemplary cardiovascular diseases and conditions include, but are not limited to, dyslipidemia, hypercholesterolemia, hypertriglyceridemia, hyperlipidemia, hypoalphalipoproteinemia, metabolic syndrome, diabetic complications, atherosclerosis, stroke, vascular dimensia, chronic kidney disease, coronary heart disease, coronary artery disease, retinopathy, inflammation, thrombosis, peripheral vascular disease, heart failure or congestive heart failure. In certain embodiments, exemplary cardiovascular diseases and conditions include, but are not limited to, hypercholesterolemia, hyperlipidemia, hyperlipoproteinemia, hypertriglyceridemia, dyslipidemia, dyslipoproteinemia, atherosclerosis, hepatic steatosis, metabolic syndrome and coronary artery disease. In certain embodiments, the disease is hypercholesterolemia, such as familial hypercholesterolemia or autosomal dominant hypercholesterolemia. In certain embodiments, the disease is hyperlipidemia. In certain embodiments, the disease is coronary artery disease.

In certain embodiments, the disclosed methods of treatment can decrease high levels of circulating serum cholesterol, such as LDL-C and VLDL-Cholesterol. In addition, the disclosed methods are useful for decreasing circulating serum triglycerides, circulating serum lipoprotein A, circulating serum LDL-C and atherogenic lipoproteins. In certain embodiments, the diseases or conditions treated with the disclosed compounds and compositions include atherosclerosis and atherosclerotic plaque formation. Subjects having a gain-of-function mutation in the PCSK9 gene also benefit with treatment with the disclosed compounds and compositions counteracting the mutation through their inhibition of PCSK9.

### Combination Treatments

Disclosed compounds and compositions may be conjointly administered with other therapeutic agents, such as other agents suitable for the treatment of high levels of LDL-C and triglycerides. In certain embodiments, conjointly administering one or more additional therapeutic agents with a compound described herein provides a synergistic effect. In certain embodiments, conjointly administering one or more additional therapeutic agents provides an additive effect.

In some embodiments in which a combination therapy is used, the amount of the compound or salt described in this specification and the amount of the other pharmaceutically active agent(s) are, when combined, therapeutically effective to treat a targeted disorder in the animal patient. In this context, the combined amounts are “therapeutically effective amounts” if they are, when combined, sufficient to reduce or completely alleviate symptoms or other detrimental effects of the disorder; cure the disorder; reverse, completely stop, or slow the progress of the disorder; or reduce the risk of the disorder getting worse. Typically, such amounts may be determined by one skilled in the art by, for example, starting with the dosage range described in this specification for the compound or salt and an approved or otherwise published dosage range(s) of the other pharmaceutically active compound(s).

A pharmaceutical composition of the specification may comprise one or more further active ingredients, as appropriate, examples of combinations of a compound of the specification (or a pharmaceutically acceptable salt thereof) and one or more additional active ingredients are described herein.

The specification further relates to a combination therapy wherein a compound of the specification, or a pharmaceutically acceptable salt thereof, and a second active ingredient are administered concurrently, sequentially or in admixture, for the treatment of one or more of the conditions listed above. Such a combination may be used in combination with one or more further active ingredients.

In one aspect there is provided a combination (for example, for use as a medicament for the treatment of one of the diseases or conditions listed herein, such as a cardiovascular disease) comprising a compound of the specification, or a pharmaceutically acceptable salt thereof, and at least one active ingredient selected from:
i) a statin;
ii) a cholesterol absorption inhibitor;
iii) a SGLT2 inhibitor;
iv) a P2Y12 inhibitor;
v) a citrate lyase inhibitor; and
vi) anti-hypertensive drugs.

In a further aspect of the present specification there is provided a pharmaceutical composition (for example, for use as a medicament for the treatment of one of the diseases or conditions listed herein, such as a cardiovascular disease) comprising a compound of the specification, or a pharmaceutically acceptable salt thereof, and at least one active ingredient selected from:
i) a statin;
ii) a cholesterol absorption inhibitor;
iii) a SGLT2 inhibitor;
iv) a P2Y12 inhibitor;
v) citrate lyase inhibitor;
vi) anti-hypertensive drugs.

In another embodiment there is provided a compound of Formula (I), or a pharmaceutically acceptable salt thereof, and at least one additional active ingredient selected from a statin, wherein the statin is selected from atorvastatin, Fluvastatin, Lovastatin, Mevastatin, Pitavastatin, Pravastatin, Rosuvastatin or Simvastatin. In another aspect the statin is Rosuvastatin (Crestor).

In another embodiment there is provided a compound of Formula (I), or a pharmaceutically acceptable salt thereof, and at least one additional active ingredient selected from a cholesterol absorption inhibitor, wherein the cholesterol absorption inhibitor is selected from Ezetimibe (Ezetrol).

In another embodiment there is provided a compound of Formula (I), or a pharmaceutically acceptable salt thereof, and at least one additional active ingredient selected from a SGLT2 inhibitor, wherein the SGLT2 inhibitor is selected from Canagliflozin, Dapagliflozin, Empagliflozin, Ertugliflozin, Ipragliflozin, Luseogliflozin, Remogliflozin etabonate, Sergliflozin etabonate, Sotagliflozin or Tofogliflozin. In some aspects the SGLT2 inhibitor is selected from Dapagliflozin (Farxiga or Forxiga).

In another embodiment there is provided a compound of Formula (I), or a pharmaceutically acceptable salt thereof, and at least one additional active ingredient selected from a P2Y12 inhibitor, wherein the P2Y12 inhibitor is selected from Ticagrelor and Clopidogrel (Plavix).

In another embodiment there is provided a compound of Formula (I), or a pharmaceutically acceptable salt thereof, and at least one additional active ingredient selected from a citrate lyase inhibitor, wherein the citrate lyase inhibitor is Bempedoic acid (Nexletol).

In another embodiment there is provided a compound of Formula (I), or a pharmaceutically acceptable salt thereof, and at least one additional active ingredient selected from Ezetimibe, Rosuvastatin, Dapagliflozin and Ticagrelor. In one embodiment there is one additional active ingredient. In another embodiment there are two additional active ingredients. In one embodiment the additional active ingredient is Ezetimibe, Rosuvastatin, Dapagliflozin or Ticagrelor. In another embodiment the additional two active ingredients are Ezetimibe and Rosuvastatin or Dapagliflozin and Rosuvastatin.

In another embodiment there is provided a compound of Formula (I), or a pharmaceutically acceptable salt thereof, and at least one additional active ingredient selected from anti-hypertensive drugs. In some aspects, the antihypertensive drug is selected from Valsartan (Diovan), Metoprolol (Lopressor), HCTZ (Hydrochlorothiazide), Olmesartan (Benicar), Lisinopril (Prinivil, Zestril), Amlodipine besylate (Norvasc), Candesartan, or a calcium channel blocker or a combination thereof. In another aspect there is provided the compound of Formula (I) or a pharmaceutically acceptable salt thereof in combination with:
i) Valsartan;
ii) Metoprolol;
iii) Valsartan and HCTZ;
iv) Olmesartan;
v) Olmesartan and HCTZ;
vi) Lisinopril;
vii) Amlodipine;
viii) Candesartan;
ix) a calcium channel blocker; or
x) HCTZ.

In one embodiment there is provided a compound of Formula (I), or a pharmaceutically acceptable salt thereof, and at least one additional active ingredient for use in the simultaneous, separate or sequential treatment of a cardiovascular disease. In one embodiment there is provided a compound of Formula (I), or a pharmaceutically acceptable salt thereof, for use in the treatment of a cardiovascular disease, where the compound of Formula (I), or a pharmaceutically acceptable salt thereof, is administered simultaneously, separately or sequentially with at least one an additional active substance selected from Ezetimibe, Rosuvastatin, Dapagliflozin and Ticagrelor.

In another embodiment there is provided a method of treating a cardiovascular disease in a subject, which comprises administering to said subject a compound of Formula (I) or a pharmaceutically acceptable salt thereof, and simultaneously, separately or sequentially administering at least one additional active substance, wherein the at least one additional active substance is selected from Ezetimibe, Rosuvastatin, Dapagliflozin and Ticagrelor.

### FURTHER EMBODIMENTS

The following embodiments may apply to all aspects as described above or may relate to a single aspect. The embodiments may be combined together in any combination.

X¹

X¹ is C—R^{A1}.

R^{A1}

In some embodiments R^{A1} is selected from the group consisting of:
(i) H
(ii) halo;
(iii) CN;
(iv) C₁₋₆ hydrocarbon, optionally substituted by one or more OH, CN, C₁₋₆ acyl, C₁₋₆ alkoxy or one or more halo groups;
(v) C₁₋₆ alkoxy, optionally substituted by OH, one or more halo groups, C₁₋₆ alkyl amido;
(vi) C₁₋₆ alkylester;
(vii) C₁₋₆ alkyl acyl; and
(viii) OH.

In some embodiments R^{A1} is selected from the group consisting of:
(i) H
(ii) halo;
(iii) CN;
(iv) C₁₋₆ hydrocarbon, optionally substituted by one or more OH, CN, C₁₋₆ acyl, C₁₋₆ alkoxy or one or more halo groups; and
(v) C₁₋₆ alkoxy, optionally substituted by OH, halo or C₁₋₆ alkyl amido.

In some embodiments R^{A1} is selected from the group consisting of:
(i) H
(ii) halo;
(iii) CN;
(iv) C₁₋₆ hydrocarbon, optionally substituted by one or more OH, CN, C₁₋₆ acyl, C₁₋₆ alkoxy or one or more halo groups;
(v) C₁₋₆ alkoxy, optionally substituted by OH, one or more halo groups or C₁₋₆ alkyl amido; and
(vi) OH.

When R^{A1} is an optionally substituted C₁₋₆hydrocarbon it is an optionally substituted C₁₋₆ alkyl. In some embodiments it is optionally substituted methyl or optionally substituted ethyl. In further embodiments, it is optionally substituted methyl. In further embodiments, it is unsubstituted methyl.

When R^{A1} is optionally substituted C₁₋₆ alkyl, in some embodiments the optional substituents are selected from OH, CN, or one or more halo groups. In further embodiments the optional substituents are selected from OH, F and Br.

When R^{A1} is optionally substituted C₁₋₆ alkoxy, in some embodiments it is optionally substituted OMe or ethoxy. In some embodiments R^{A1} is unsubstituted OMe.

When R^{A1} is optionally substituted C₁₋₆ alkoxy, in some embodiments the optional substituents are selected from alkyl amido or one or more halo groups. In another embodiment the optional substituents are selected from one, two or three F atoms.

When R^{A1} is halo, in some embodiments it is F, Br or Cl. In other embodiments it is Br or Cl.

In some embodiments R^{A1} is OH.

In some embodiments R^{A1} is CN.

In some embodiments R^{A1} is methyl.

In some embodiments R^{A1} is —OCF₂H.

In some embodiments R^{A1} is selected from H, Br, Cl, CN, OMe, ethoxy, methyl or ethyl. In further embodiments R^{A1} is selected from H, —OCF₂H, Br and Cl. In further embodiments R^{A1} is selected from H, Br or Cl. In other embodiments R^{A1} is H or —OCF₂H.

In some embodiments R^{A1} is selected from the group consisting of H, OH, Br, Cl, CN, OCF₂H, OMe, ethoxy, methyl and ethyl. In some embodiments R^{A1} is selected from the group consisting of H, Br, Cl, CN, OCF₂H, OMe, ethoxy, methyl and ethyl. In some embodiments R^{A1} is selected from the group consisting of H, OH, —OCF₂H, Br and CI. In some embodiments R^{A1} is selected from the group consisting of H, —OCF₂H, Br and C.

In some embodiments R^{A1} is H.

In some embodiments R^{A1} is OH or H.

In some embodiments R^{A2} is selected from the group consisting of:
(i) H;
(ii) halo;
(iii) CN;
(iv) C₁₋₆ hydrocarbon, optionally substituted by OH, CN, C₁₋₆ acyl, C₁₋₆ alkoxy or one or more halo groups;
(v) C₁₋₆ alkoxy, optionally substituted by OH, alkyl amido, or one or more halo groups;
(vi) C₁₋₆ acyl amido (wherein the acyl is optionally substituted by H or methyl);
(vii) C₁₋₆ thioalkyl, optionally substituted by C₁₋₆ alkyl ester;
(viii) C₁₋₆ alkyl ester;
(ix) C₁₋₆ alkyl acyl,
(x) C₄₋₅ heterocyclyl;
(xi) C₅heteroaryl;
(xii) C₁₋₆ alkyl amido optionally substituted by C₁₋₃ alkyl amido, CN, OH, C₂₋₃ alkynyl, C₄₋₆ heterocyclyl, C₁₋₃ alkyl which alkyl is optionally substituted with one or more halo or OH groups; and
(xiii) OH.

In some embodiments R^{A2} is selected from the group consisting of:
(i) H;
(ii) halo;
(iii) CN;
(iv) C₁₋₆ hydrocarbon, optionally substituted by OH, CN, C₁₋₆acyl, C₁₋₆ alkoxy or one or more halo groups;
(v) C₁₋₆ alkoxy, optionally substituted by OH, alkyl amido, or one or more halo groups;
(vi) C₁₋₆ acyl amido (wherein the acyl is optionally substituted by H or methyl);
(vii) C₁₋₆ thioalkyl,
(viii) C₁₋₆ alkyl ester;
(ix) C₁₋₆ alkyl acyl,
(x) C₄₋₅ heterocyclyl;
(xi) C₅heteroaryl;
(xii) C₁₋₆ alkyl amido optionally substituted by C₁₋₃alkyl amido, CN, C₂₋₃ alkynyl, C₄₋₆ heterocyclyl, C₁₋₃ alkyl which alkyl is optionally substituted with one or more halo or OH groups; and
(xiii) OH.

In further embodiments R^{A2} is selected from the group consisting of:
(i) H;
(ii) halo;
(iii) CN;
(iv) C₁₋₆ hydrocarbon, optionally substituted by OH, CN, C₁₋₆acyl, C₁₋₆ alkoxy or one or more halo groups;
(v) C₁₋₆ alkoxy, optionally substituted by OH, C₁₋₆ alkyl amido, or one or more halo groups;
(vi) C₁₋₆ thioalkyl;
(viii) C₁₋₆ alkyl ester; and
(ix) C₁₋₆ alkyl amido optionally substituted by C₁₋₃ alkyl amido, CN, C₂₋₃ alkynyl, C₄₋₆ heterocyclyl, or C-alkyl which alkyl is optionally substituted with one or more halo or OH groups.

In further embodiments R^{A2} is selected from the group consisting of:
(i) H;
(ii) halo;
(iii) CN;
(iv) C₁₋₆ hydrocarbon, optionally substituted by OH, CN, C₁₋₆ acyl, C₁₋₆alkoxy or one or more halo groups;
(v) OH;
(vi) C₁₋₆ alkoxy, optionally substituted by OH, C₁₋₆ alkyl amido, or one or more halo groups;
(vii) C₁₋₆ alkyl ester;
(viii) C₁₋₆ alkyl acyl; and
(ix) C₁₋₆ alkyl amido optionally substituted by C₁₋₃ alkyl amido, CN, C₂₋₃ alkynyl, C₄₋₆ heterocyclyl, or C₁₋₃ alkyl which alkyl is optionally substituted with one or more halo or OH groups.

In some embodiments R^{A2} is selected from:
(i) H;
(ii) halo;
(iii) CN;
(iv) C₁₋₆ hydrocarbon, optionally substituted by OH, CN, C₁₋₆ acyl, C₁₋₆alkoxy or one or more halo groups;
(v) OH;
(vi) C₁₋₆ alkoxy, optionally substituted by OH, C₁₋₆ alkyl amido, or one or more halo groups;
(vii) C₁₋₆ alkyl ester;
(viii) C₁₋₆ alkyl acyl;
(ix) C₁₋₆ alkyl amido optionally substituted by C₁₋₃ alkyl amido, CN, C₂₋₃ alkynyl, C₄₋₆ heterocyclyl, or C₁₋₃ alkyl which alkyl is optionally substituted with one or more halo or OH groups; and (x) C₁₋₆ thioalkyl optionally substituted by C₁₋₆ alkyl ester.

In some embodiments R^{A2} is selected from:
(i) H;
(ii) halo;
(iii) CN;
(iv) C₁₋₆ hydrocarbon, optionally substituted by OH, CN, C₁₋₆acyl, C₁₋₆alkoxy or one or more halo groups;
(v) C₁₋₆ alkoxy, optionally substituted by OH, alkyl amido, or one or more halo groups;
(vi) C₁₋₆ thioalkyl, optionally substituted by C₁₋₆ alkyl ester;
(vii) C₁₋₆ alkyl ester; or
(viii) C₁₋₆ alkyl amido optionally substituted by C₁₋₃ alkyl amido, CN, C₂₋₃ alkynyl, C₄₋₆heterocyclyl or C₁₋₃alkyl wherein the C₁₋₃ alkyl is optionally substituted with one or more halo or OH groups.

In some embodiments R^{A2} is selected from:
(i) H;
(ii) halo;
(iii) CN;
(iv) C₁₋₆ alkyl;
(v) cyclopropyl;
(vi) C₁₋₆ alkoxy, optionally substituted by one or more halo groups;
(vii) C₁₋₆ alkyl ester;
(viii) C₁₋₆alkyl amido;
(ix) C₁₋₆ thioalkyl, optionally substituted by C₁₋₆ alkyl ester; or
(x) OH.

In further embodiments R^{A2} is selected from:
(i) H;
(ii) Cl;
(iii) CN;
(iv) methyl;
(v) cyclopropyl;
(vii) C₁₋₂ alkoxy, optionally substituted by two or three fluoro groups;
(viii) C₁₋₃ thioalkyl, optionally substituted by C₁₋₃ alkyl ester;
(viii) C(═O)CH₃; or
(ix) C(═O)NH(CH₃).

When R^{A2} is halo, in some embodiments it is Br or Cl. In further embodiments it is Cl.

In some embodiments R^{A2} is selected from CN, Cl, OMe, methyl, cyclopropyl, —OCF₂H, —OCF₃ and optionally substituted C₁₋₆ alkylamido.

In some embodiments R^{A2} is CN.

In some embodiments R^{A2} is H.

In some embodiments R^{A2} is OH.

In some embodiments R^{A2} is —C(═O)CH₃.

In some embodiments R^{A2} is —OCF₂H.

In some embodiments R^{A2} is cyclopropyl.

In some embodiments R^{A2} is Cl.

In some embodiments R^{A2} is methyl.

In some embodiments R^{A2} is —S—CH₃.

In some embodiments R^{A2} is —S—CH₂CH₃.

In some embodiments R^{A2} is —S—CH₂—C(═O)—O—CH₃.

In some embodiments R^{A2} is C(═O)NH(CH)₃.

When R^{A2} is a C₁₋₆ hydrocarbon it is an optionally substituted C₁₋₆ alkyl. In some embodiments it is optionally substituted methyl, optionally substituted ethyl or optionally substituted cyclopropyl.

In further embodiments, it is optionally substituted methyl. In further embodiments, it is unsubstituted methyl. In other embodiments it is unsubstituted cyclopropyl.

When R^{A2} is optionally substituted C₁₋₆ alkyl, in some embodiments the optional substituents are selected from OH, CN, or one or more halo groups. In further embodiments the optional substituents are selected from OH, F and Br.

When R^{A2} is optionally substituted C₁₋₆ alkoxy, in some embodiments it is optionally substituted OMe or ethoxy.

When R^{A2} is optionally substituted C₁₋₆ alkoxy, in some embodiments the optional substituents are selected from alkyl amido or one or more halo groups. In another embodiment the optional substituents are selected from one or more F. In another embodiment where R^{A2} is optionally substituted C₁₋₆ alkoxy it is difluoromethoxy (OCHF₂).

When R^{A2} is C₁₋₆ alkyl ester, in some embodiments it is —C(═O)OCH₂CH₃.

When R^{A2} is C₁₋₆ alkyl amido, in some embodiments the optional substituents are selected from one or more methyl groups, an oxetane ring, a C₂alkylamido, ethyl which ethyl is optionally substituted by OH or one or more halo groups. In other embodiments when R^{A2} is C₁₋₆ alkyl amido it is C(═O)NHCH₂C(═O)NH₂, C(═O)NHCH₂CHCH, —C(═O)NH-oxetane, C(═O)NHCH₂CHF₂, C(═O)NHCH₂CH₂OH, C(═O)NHCH₂CH₃, C(═O)NH₂, C(═O)NHCH₃, C(═O)N(CH₃)₂. When R^{A2} is C₁₋₆ alkyl amido, in some embodiments the optional substituents are OH.

When R^{A2} is C₁₋₆ thioalkyl, in some embodiments the optional substituents are a C₁₋₆ alkyl ester. When R^{A2} is C₁₋₆ thioalkyl, in some embodiments the optional substituents are selected from C(═O)—O—CH₃ or C(═O)—O—CH₂CH₃. In some embodiments the thioalkyl is unsubstituted and is —S—CH₃ or —S—CH₂CH₃. In some embodiments when R^{A2} is an optionally substituted C₁₋₆ thioalkyl, it is S—CH₃, —S—CH₂CH₃ or —S—CH₂—C(═O)—O—CH₃.

In other embodiments R^{A2} is selected from —OCHF₂, Cl, —OMe, methyl, C(═O)CH₃, CN, —CH₂OH, H and cyclopropyl. In further embodiments R^{A2} is selected from methyl, —OCHF₂, Cl, —CH₂OH, H, CN, —C(═O)CH₃, —OMe.

In further embodiments R^{A2} is selected from methyl, —OCHF₂, Cl and cyclopropyl.

In further embodiments R^{A2} is selected from H, —COOH, —CH₂OH, methyl, CN, cyclopropyl, —C(═O)CH₃, —OCF₂H, Cl, —C(═O)OCH₂CH₃, —C(═O)NH₂, —C(═O)NHCH₃, —C(═O)N(CH₃)₂, —C(═O)NHCH₂C(═O)NH₂, —C(═O)NHCH₂CHCH, —C(═O)NH-oxetane, —C(═O)NHCH₂CHF₂, —C(═O)NHCH₂CH₂OH, —C(═O)NHCH₂CH₃.

In further embodiments R^{A2} is selected from the group consisting of —CN, methyl, Cl, —C(═O)CH₃, —C(═O)OCH₂CH₃, cyclopropyl, —C(═O)NHCH₂C(═O)NH₂, —C(═O)NHCH₂CHCH, —C(═O)NH-oxetane, —C(═O)NHCH₂CHF₂, —C(═O)NHCH₂CH₂OH, —C(═O)NHCH₂CH₃, —C(═O)NH₂, —C(═O)NHCH₃, —C(═O)N(CH₃)₂, —OCF₂H, H, —OMe, and —OCF₃

In further embodiments R^{A2} is selected from CN, methyl, Cl, —C(═O)CH₃, —OCHF₂, cyclopropyl, —OCF₃, —OCH₃, H, —C(═O)NH(CH₃), —S—CH₃, —S—CH₂CH₃ or —S—CH₂—C(═O)—O—CH₃.

In some embodiments R^{A2} is selected from CN, methyl, Cl, —C(═O)CH₃, —C(C═O)OCH₂CH₃, cyclopropyl, —C(═O)NHCH₂C(═O)NH₂, —C(═O)NHCH₂CHCH, —C(═O)NH-oxetane, —C(═O)NHCH₂CHF₂, —C(═O)NHCH₂CH₂OH, —C(═O)NHCH₂CH₃, —C(═O)NH₂, —C(═O)NHCH₃, —C(═O)N(CH₃)₂, —OCF₂H, H, —OMe, —OCF₃. These groups are as shown in the table below:

| | |
|---|---|
| 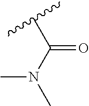 [SMILES: *C(=O)N(C)C] | 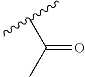 [SMILES: *C(C)=O] |
| 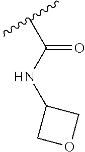 [SMILES: *C(=O)NC1COC1] | 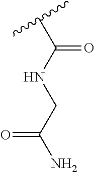 [SMILES: *C(=O)NCC(N)=O] |
| 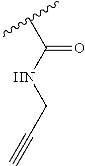 [SMILES: *C(=O)NCC#C] | 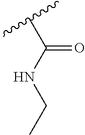 [SMILES: *C(=O)NCC] |
| 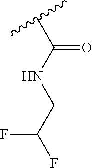 [SMILES: *C(=O)NCC(F)F] | 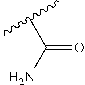 [SMILES: *C(N)=O] |
| 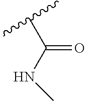 [SMILES: *C(=O)NC] | 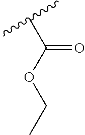 [SMILES: *C(=O)OCC] |
| 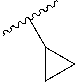 [SMILES: *C1CC1] | 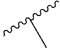 |
| 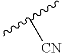 [SMILES: *C#N] | 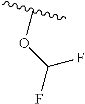 [SMILES: *OC(F)F] |
| 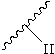 [SMILES: *[H]] | 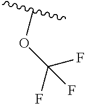 [SMILES: *OC(F)(F)F] |
| 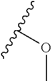 [SMILES: *OC] | 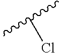 [SMILES: *Cl] |

In some embodiments R^{A2} is selected from the following groups:

[symbol]

In some embodiments R^{A2} is selected from the following groups:

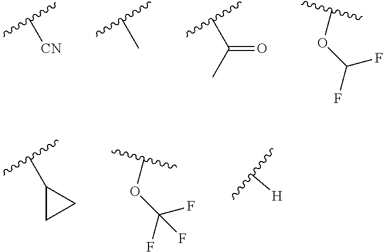

In some embodiments R^{A3} is selected from the group consisting of:
(i) H;
(ii) halo;
(iii) ON;
(iv) C₁₋₆hydrocarbon, optionally substituted by OH, ON, C₁₋₆ thioalkyl, C₁₋₆ alkoxy, C₁₋₆ alkylacyl, C₁₋₆ acyloxy, carboxy, C₁₋₆ alkylester, C₁₋₆ alkylamino; —C(═O)NH₂, C₁₋₆ alkyl amido, C₁₋₆ alkylacylamido, C₁₋₆ alkyl sulfinyl, C₁₋₆ alkyl sulfonyl or one or more halo groups;
(v) OH;
(vi) C₁₋₆ alkoxy, optionally substituted by OH, NH₂, C₄ heterocyclyl or one or more halo groups.

Where R^{A3} is halo, in some embodiments it is Br or Cl. In some embodiments it is Cl. In further embodiments R^{A3} is Br.

Where R^{A3} is a C₁₋₆hydrocarbon it is an optionally substituted C₁₋₆ alkyl. In some embodiments it is optionally substituted methyl or optionally substituted ethyl. In further embodiments, it is optionally substituted methyl. In further embodiments, it is unsubstituted methyl.

When R^{A3} is optionally substituted C₁₋₆ alkyl, in some embodiments the optionally substituents are selected from OH, CN, or one or more halo groups. In further embodiments the optional substituents are selected from OH, F and Br.

In some embodiments R^{A3} is OH.

When R^{A3} is optionally substituted C₁₋₆ alkoxy, in some embodiments it is optionally substituted OMe or ethoxy. In further embodiments it is OMe.

When R^{A3} is optionally substituted C₁₋₆ alkoxy, in some embodiments the optional substituents are selected from alkyl amido or one or more halo groups. In another embodiment the optional substituents are selected from one or more F.

In some embodiments R^{A3} is selected from H, CF₃, CN, C₁₋₂alkyl, NH₂ and halo. In other embodiments R^{A3} is selected from H, methyl, CN and C₁.

In some embodiments R^{A3} is selected from H, OMe, CF₃, CN, C₁₋₂alkyl, NH₂ and halo.

In some embodiments R^{A3} is CN.

In some embodiments R^{A3} is H.

In some embodiments R^{A3} is methyl.

In some embodiments R^{A3} is OMe.

In some embodiments R^{A3} is selected from methyl, H and CN.

R^{A2} and R^{A3}

When R^{A3} and R^{A2} together with the carbon atoms to which they are bound form an optionally substituted C carboaromatic ring or C₅₋₇ heteroaromatic ring they form an optionally substituted benzene ring or an optionally substituted pyridine ring.

When R^{A3} and R^{A2} together with the carbon atoms to which they are bound form an optionally substituted C carboaromatic ring or C₅₋₇ heteroaromatic ring the optional substituents are selected from NH₂, C₁₋₆ alkyl, C₁₋₆ alkoxy and halo. In other embodiments the optional substituents are selected from methyl, ethyl, OMe, NH₂, F, Cl and Br. In other embodiments the optional substituents are selected from methyl, NH₂, Cl, F and OMe. In other embodiments the optional substituent is methyl.

In one embodiment when R^{A2} and R^{A1} together with the carbon atoms to which they are bound form an optionally substituted C₅₋₇ heteroaromatic ring, they form an optionally substituted pyridine. In some embodiments the optional substituent is NH₂. In another embodiment R^{A2} and R^{A3} together with the carbon atoms to which they are bound form an unsubstituted pyridine. In another embodiment R^{A} and R^{A3} together form an optionally substituted pyrazole, an optionally substituted pyrrole or an optionally substituted thiazole. In some embodiments the optional substituent is methyl.

When R^{A3} and R^{A2} together with the carbon atoms to which they are bound form an optionally substituted C₅₋₇ heteroaromatic ring, the optional substituents are selected from C₁₋₆ alkyl, C₁₋₆ alkoxy, NH₂ and halo. In other embodiments the optional substituents are selected from methyl, ethyl, OMe, ethoxy, NH₂ and halo. In other embodiments the optional substituents are selected from NH₂ and methyl.

When R^{A3} and R^{A2} together with the carbon atoms to which they are bound form an optionally substituted C₅₋₇ heterocycle ring, they form a 5 membered ring which comprises one or two atoms selected from N, O and S. In some embodiments the 5 membered ring contains one N and one S. In other embodiments the 5 membered ring contains one N. In other embodiments the 5 membered ring contains one N and one O. In other embodiments the 5 membered ring contains two Ns. In some embodiments R^{A3} and R^{A} together with the carbon atoms to which they are bound form an optionally substituted pyrrole or pyrazole.

When R^{A3} and R^{A2} together with the carbon atoms to which they are bound form an optionally substituted C₅₋₇ heterocycle ring, the optional substituents are selected from NH₂, C₁₋₆alkyl, C₁₋₆alkoxy and halo. In other embodiments the optional substituents are selected from methyl, ethyl, OMe, ethoxy, NH₂, F, Cl and Br. In other embodiments the optional substituent is methyl.

In other embodiments R^{A2} and R^{A3} together with the carbon atoms to which they are bound form:
(i) optionally substituted C₆ heteroaromatic ring; wherein the optional substituent is NH₂;
(ii) optionally substituted C₆ carboaromatic ring; wherein the optional substituent is F, OMe, C₁;
(iii) optionally substituted C₅ heteroaromatic or C₅ heterocycle ring wherein the optional substituent is methyl.

In some embodiments R^{A3} and R^{A2} together with the carbon atoms to which they are bound form an optionally substituted C carboaromatic ring or C₅₋₇ heteroaromatic ring wherein the optional substituents are selected from C₁₋₆alkyl, and halo.

In some embodiments R^{A2} and R^{A3} together form an unsubstituted 2-pyrazole, a 2-pyrrole substituted by methyl, pyridine optionally substituted by NH₂, or a phenyl optionally substituted by Cl, F or OMe.

In some embodiments R^{A2} and R^{A3} together form a ring selected from:

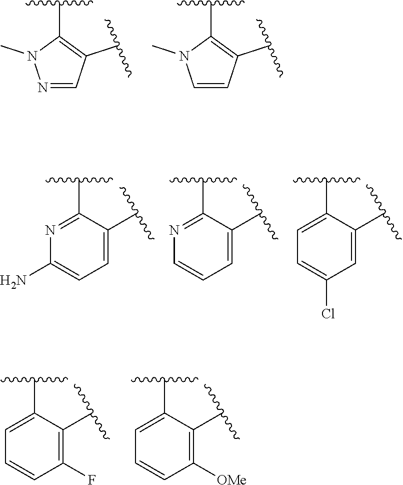

### B

In some embodiments B is of formula (B-1):

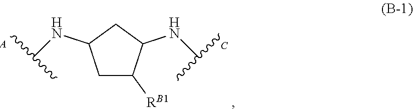

wherein the wavy lines indicate the point of attachment to A and C; wherein R^{B1} is H, OH, —OMe, —O-Ethyl, —CH₂OH, —CH₂CH₂OH or ═CHCH₂—OH.

In other embodiments R^{B1} is H, —CH₂OH, —CH₂CH₂OH or ═CHCH₂—OH.

In other embodiments R^{B}1 is —CH₂OH, —CH₂CH₂OH or ═CHCH₂—OH.

In other embodiments R^{B1} is H.

In another embodiment B is of the formula (B-1a):

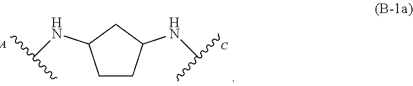

In further embodiments B is of the formula (B-1b):

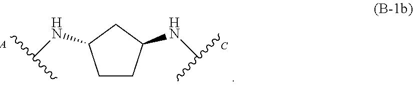

Therefore in some embodiments the compounds of Formula (I) is the S,S-enantiomer.

In some embodiments B is of formula (B-2):

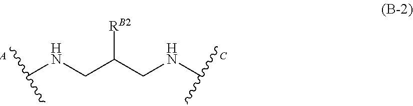

wherein the wavy line indicates the point of attachment to A and C;

R^{B2} is C₁₋₂ alkyl-OH, CH₂CONHMe or C₁₋₃ alkyl.

In some of these embodiments, R^{B2} is C₁₋₂ alkyl-OH, or C₁₋₃ alkyl In some embodiments when B is of the formula (B-2) it is of the following formula (B-2a) wherein the wavy line indicates the point of attachment to A and C; and R^{B2} is C₁₋₂alkyl-OH, CH₂CONHMe or C₁₋₂alkyl;

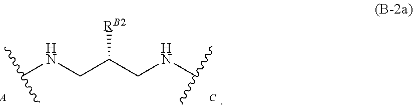

### C

C is selected from the group consisting of C₆₋₁₀ carboaryl, C₅₋₆ heteroaryl and C₅₋₁₀ heterocyclyl, which groups are optionally substituted by:
(i) C₆₋₁₀ carboaryl, C₄₋₁₀ carbocyclyl, C₅₋₁₀ heteroaryl C₄₋₁₀ heterocyclyl, or C₅₋₁₀ bridged heterocycle, spiro C₆₋₁₂heterocyclyl or a spiro C₆₋₁₂carbocyclyl,

which are themselves optionally substituted by one or more of the following groups:
a) one or two ═O groups;
b) one or more halo groups;
c) CN, NH₂, OH;
d) one or more C₁₋₆ alkyl groups including branched and cyclic and with an optional substituent selected from OH, C₁₋₆ alkyl sulfonyl or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl with an optional methyl substituent;
i) C₄₋₁₀ carbocyclyl with an optional methyl or ═O substituent;
j) C₆₋₁₀carboaryl with optional substituents of one or more halo groups;
l) P(═O)Me₂;
m) carboxy or CH₂-carboxy;
n) tetrazolyl, CH₂-tetrazolyl, 5-oxo-4H-1,2,4-oxadiazol-3-yl;
(ii) one or more groups selected from carboxy, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido, and di-C₁₋₆ alkyl sulfonamido.

In some embodiments C is selected from the group consisting of C₆₋₁₀ carboaryl, C₅₋₆ heteroaryl and C₅₋₁₀ heterocyclyl, which groups are optionally substituted by:
(i) C₆₋₁₀ carboaryl, C₄₋₁₀ carbocyclyl, C₅₋₁₀ heteroaryl C₄₋₁₀ heterocyclyl, or C₅₋₁₀ bridged heterocycle, spiro C₆₋₁₂heterocyclyl or a spiro C₆₋₁₂carbocyclyl, which are themselves optionally substituted by one or more of the following groups:
a) one or two ═O groups;
b) one or more halo groups;
c) CN, NH₂, OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl;
i) C₄₋₁₀ carbocyclyl with an optional methyl or ═O substituent;
j) C₆₋₁₀ carboaryl with optional substituents of one or more halo groups;
l) P(═O)Me₂;
m) carboxy or CH₂-carboxy; and/or
n) tetrazolyl, CH₂-tetrazolyl, 5-oxo-4H-1,2,4-oxadiazol-3-yl;
(ii) one or more groups selected from carboxy, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido, and di-C₁₋₆ alkyl sulfonamido.

In some embodiments C is an optionally substituted pyridinyl, pyrazinyl or pyrimidinyl, wherein the optional substituents are selected from C₆₋₁₀ carboaryl, C₄₋₁₀ carbocyclyl, C₅₋₁₀ heteroaryl, C₅₋₁₀ heterocyclyl, C₅₋₁₀ bridged heterocyclyl, spiro C₆₋₁₂heterocyclyl or a spiro C₆₋₁₂carbocyclyl, which are themselves optionally substituted by one or more of the following groups:
a) one or two ═O groups;
b) one or more halo groups;
c) CN, NH₂ or OH;
d) one or more C₁₋₆ alkyl groups including branched and cyclic and with an optional substituent selected from OH, C₁₋₆ alkyl sulfonyl or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl with an optional methyl substituent;
i) C₄₋₁₀ carbocyclyl with an optional methyl or ═O substituent;
j) C₆₋₁₀ carboaryl with optional substituents of one or more halo groups;
l) P(═O)Me₂;
m) carboxy or CH₂-carboxy; and/or
n) tetrazolyl, CH₂-tetrazolyl, 5-oxo-4H-1,2,4-oxadiazol-3-yl.

In some embodiments C is an optionally substituted pyridinyl, pyrazinyl or pyrimidinyl, wherein the optional substituents are selected from C₆₋₁₀ carboaryl, C₄₋₁₀ carbocyclyl, C₅₋₁₀ heteroaryl, C₅₋₁₀ heterocyclyl, C₅₋₁₀ bridged heterocyclyl, spiro C₆₋₁₂ heterocyclyl or a spiro C₆₋₁₂carbocyclyl, which are themselves optionally substituted by one or more of the following groups:
a) one or two ═O groups;
b) one or more halo groups;
c) CN, NH₂ or OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl;
i) C₄₋₁₀ carbocyclyl with an optional methyl or ═O substituent;
j) C₆₋₁₀ carboaryl with optional substituents of one or more halo groups;
l) P(═O)Me₂;
m) carboxy or CH₂-carboxy; and/or
n) tetrazolyl, CH₂-tetrazolyl, 5-oxo-4H-1,2,4-oxadiazol-3-yl.

When C is an optionally substituted C₅₋₆ heteroaryl in some embodiments it is an optionally substituted C₆heteroaryl. In other embodiments it is an optionally substituted pyridinyl, pyrazinyl or pyrimidinyl. In other embodiments it is an optionally substituted pyridinyl.

When C is an optionally substituted C₅₋₆ heteroaryl it can be optionally substituted by:
(i) C₆₋₁₀ carboaryl, C₄₋₁₀ carbocyclyl, C₅₋₁₀ heteroaryl, C₅₋₁₀ heterocyclyl, or C₅₋₁₀ bridged heterocyclyl, spiro C₆₋₁₂heterocyclyl or a spiro C₆₋₁₂carbocyclyl, which are themselves optionally substituted by one or more of the groups selected from:
a) one or two ═O groups;
b) one or more halo groups;
c) CN, NH₂, OH;
d) C₁₋₆ alkyl groups including branched and cyclic and with an optional substituent selected from OH, C₁₋₆ alkyl sulfonyl or one or more halo groups;
e) C₁₋₆ alkoxy with optional substituents of one or more halo groups;
f) C₁₋₆alkylester;
g) C₅₋₆ heterocyclyl with an optional methyl, OH or ═O substituent;
h) C₅₋₆ heteroaryl with an optional methyl substituent;
i) C₄₋₁₀carbocyclyl with an optional methyl or ═O substituent;
j) C₆₋₁₀carboaryl with optional substituents of one or more halo groups;
l) P(═O)Me₂;
m) carboxy or CH₂-carboxy; and/or
n) tetrazolyl, CH₂-tetrazolyl, 5-oxo-4H-1,2,4-oxadiazol-3-yl;
(ii) one or more groups selected from carboxy, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido and di-C₁₋₆ alkyl sulfonamido.

Where C is substituted by C₆₋₁₀ carboaryl, C₅₋₁₀ heteroaryl or C₅₋₁₀ heterocyclyl, it may bear a number of substituent groups. The substituents are selected from:
a) one or two ═O groups;
b) one or more halo groups, CN, NH₂;
c) one or more C₁₋₆ alkyl including (CH₃)₂, C₁₋₆ alkoxy, or C₁₋₆alkylester, wherein each is optionally substituted by one or more halo groups;
d) C₅₋₆ heterocyclyl or C₅₋₆heteroaryl with an optional methyl substituent;
e) C₆₋₁₀carboaryl optionally substituted by one or more halo atoms;
f) P(═O)Me₂; or
g) carboxy or CH₂-carboxy.

Where C is substituted by C₆₋₁₀ carboaryl, C₅₋₁₀ heteroaryl, or C₅₋₁₀ heterocyclyl, it may bear a number of substituent groups. The substituents are selected from:
a) one or two ═O groups;
b) one or more halo groups, CN or NH₂;
c) one or more C₁₋₆ alkyl groups, C₁₋₆ alkoxy, or C₁₋₆alkylester, wherein each group is optionally substituted by one or more halo groups;
d) C₅₋₆ heterocyclyl or C₅₋₆ heteroaryl with an optional methyl substituent;
e) C₆₋₁₀ carboaryl optionally substituted by one or more halo atoms;
f) carboxy or CH₂-carboxy; or
g) tetrazolyl, CH₂-tetrazolyl, 5-oxo-4H-1,2,4-oxadiazol-3-yl.

Where C is substituted by C₆₋₁₀ carboaryl, C₅₋₁₀ heteroaryl or C₅₋₁₀ heterocyclyl, it may bear a number of substituent groups. The substituents are selected from:
a) one or two ═O groups;
b) one or more halo groups,
c) CN;
d) one or more C₁₋₆ alkyl optionally substituted by one or more halo groups or C₁₋₆ alkyl sulfonyl;
e) C₁₋₆ alkoxy optionally substituted by one or more halo groups;
f) C₅₋₆ heterocyclyl or C₅₋₆ heteroaryl with an optional methyl substituent;
g) C₆ carboaryl optionally substituted by one or more halo atoms; or
h) carboxy.

In some embodiments when C is an optionally substituted C₅₋₆heteroaryl, the optional substituents are selected from C₁₋₆alkyl and halo. In further embodiments the optional substituent is methyl. In further embodiments C is substituted by methyl at the meta position. In some embodiments C is a C₆₋₁₀ carboaryl, C₅₋₆ heteroaryl and C₅₋₁₀ heterocyclyl, wherein each group is substituted by methyl.

In some embodiments one substituent of C is at the para position.

In other embodiments where C is substituted by an optionally substituted C₆₋₁₀ carboaryl, C₅₋₁₀ heteroaryl or C₅₋₁₀ heterocyclyl, it may be substituted by an optionally substituted phenyl, an optionally substituted pyridyl, an optionally substituted pyridazine, an optionally substituted imidazolidinyl, an optionally substituted pyrrolidine, an optionally substituted dihydroquinolinyl, an optionally substituted napthyridine, an optionally substituted 2,3-dihydro-1H-imidazo[4,5-b]pyridine, an optionally substituted 3H-imidazo[4,5-b]pyridine, an optionally substituted benzimidazolyl or an optionally substituted imidazopyridinyl. In other embodiments where C is substituted by an optionally substituted C₆₋₁₀ carboaryl, C₅₋₁₀ heteroaryl or C₅₋₁₀ heterocyclyl, it may be substituted by an optionally substituted phenyl, an optionally substituted pyridyl, an optionally substituted imidazolidinyl, an optionally substituted dihydroquinolinyl, an optionally substituted benzimidazolyl or an optionally substituted imidazopyridinyl. These groups themselves may be optionally substituted by one or two ═O groups, halo, CN, one or more C₁₋₆ alkyl, C₁₋₆ alkoxy, C₁₋₆alkylester, C₅₋₆ heterocyclyl (with an optional methyl substituent), phenyl substituted by F at the para position, carboxy, CH₂-carboxy, tetrazolyl, pyrazolyl, triazolyl or P(═O)Me₂. In other embodiments the optionally substituted phenyl, optionally substituted pyridyl, optionally substituted imidazolidinyl, optionally substituted dihydroquinolinyl, optionally substituted benzimidazolyl or optionally substituted imidazopyridinyl may be substituted by one or two ═O groups, halo, CN, one or more C₁₋₆ alkyl optionally substituted by one or more halo groups or C₁₋₆ alkyl sulfonyl, C₁₋₆ alkoxy optionally substituted by one or more halo groups, C₅₋₆ heterocyclyl (with an optional methyl substituent), C₅₋₆ heteroaryl (with an optional methyl substituent), phenyl substituted by F or carboxy.

In other embodiments when C is an optionally substituted pyridinyl, pyrazinyl or pyrimidinyl it may bear a number of substituent groups. The substituents are selected from an optionally substituted C₅₋₁₀ heteroaryl and C₅₋₁₀ heterocyclyl, which are themselves optionally substituted by one or two ═O groups, one or more halo groups, CN, one or more C₁₋₆ alkyl, C₁₋₆ alkoxy, C₁₋₆alkylester, C₅₋₆ heterocyclyl (with an optional methyl substituent), phenyl optionally substituted with one or two halo groups, carboxy, CH₂-carboxy, tetrazolyl, pyrazolyl, triazolyl, a pyridine ring, NH₂ or OH. In other embodiments the optionally substituted C₅₋₁₀ heteroaryl and C₅₋₁₀ heterocyclyl, may be optionally substituted by one or two ═O groups, halo, CN, one or more C₁₋₆ alkyl optionally substituted by one or more halo groups or C₁₋₆ alkyl sulfonyl, C₁₋₆ alkoxy optionally substituted by one or more halo groups, C₅₋₆ heterocyclyl (with an optional methyl substituent), C₅₋₆ heteroaryl (with an optional methyl substituent), phenyl substituted by F or carboxy. In some embodiments the substituents are an optionally substituted phenyl, an optionally substituted pyridyl, an optionally substituted imidazolidine, an optionally substituted dihydroquinoline, an optionally substituted benzimidazolyl or an optionally substituted imidazopyridinyl. In some embodiments the substituents are an optionally substituted pyridyl, an optionally substituted pyridazine, an optionally substituted imidazolidinyl, an optionally substituted pyrrolidinyl, an optionally substituted dihydroquinolinyl, an optionally substituted napthyridine, an optionally substituted benzimidazolyl, an optionally substituted 2,3-dihydro-1H-imidazo[4,5-b]pyridine, an optionally substituted 3H-imidazo[4,5-b]pyridine, and optionally substituted 3-azabicyclo[3.1.0]hexane or an optionally substituted 2,3,3a,4,5,6,7,7a-octahydrofuro[2,3-c]pyridine.

In some embodiments C is an optionally substituted pyridinyl, pyrazinyl or pyrimidinyl wherein the optional substituents are selected from:
i) C₆₋₁₀carboaryl, C₅₋₁₀ heteroaryl C₅₋₁₀ heterocyclyl, which are themselves optionally substituted by one or more of the following groups:
a) one or two ═O groups;
b) one or more halo groups;
c) CN, NH₂, OH;
d) one or more C₁₋₆ alkyl groups including branched and cyclic and with an optional substituent selected from OH, C₁₋₆ alkyl sulfonyl or one or more halo groups; e) C₁₋₆ alkoxy with optional substituents of one or more halo groups;
f) C₁₋₆ alkylester;
g) C₅₋₆ heterocyclyl with an optional methyl, OH or ═O substituent; h) C₅₋₆ heteroaryl;
i) C₄₋₁₀ carbocyclyl with an optional methyl or ═O substituent;
j) C₆₋₁₀carboaryl with optional substituents of one or more halo groups;
l) P(═O)Me₂;
m) carboxy, CH₂-carboxy; and/or
n) tetrazolyl, CH₂-tetrazolyl, 5-oxo-4H-1,2,4-oxadiazol-3-yl;
ii) one or more groups selected from carboxy, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido and di-C₁₋₆ alkyl sulfonamido.

In some embodiments C is an optionally substituted pyridinyl, pyrazinyl or pyrimidinyl wherein the optional substituents are selected from C₆₋₁₀carboaryl, C₅₋₁₀ heteroaryl C₅₋₁₀ heterocyclyl, which are themselves optionally substituted by one or more groups selected from the following:
a) one or two ═O groups;
b) one or more halo groups;
c) CN, NH₂, OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl;
i) C₄₋₁₀ carbocyclyl with an optional methyl or ═O substituent;
j) C₆₋₁₀carboaryl with optional substituents of one or more halo groups;
l) P(═O)Me₂;
m) carboxy, CH₂-carboxy; and/or
n) tetrazolyl, CH₂-tetrazolyl, 5-oxo-4H-1,2,4-oxadiazol-3-yl.

In some embodiments C is an optionally substituted pyridinyl wherein the optional substituents are selected from C₆₋₁₀ carboaryl, C₅₋₁₀ heteroaryl, C₅₋₁₀ heterocyclyl, which are themselves optionally substituted by one or more groups selected from the following:
a) one or two ═O groups;
b) one or more halo groups,
c) CN;
d) one or more C₁₋₆ alkyl, optionally substituted by sulfonyl or one or more halo groups;
e) C₁₋₆ alkoxy optionally substituted by one or more halo groups;
f) C₅₋₆ heterocyclyl or C₅₋₆heteroaryl which groups have an optional methyl substituent;
g) C₆carboaryl optionally substituted by one or more halo atoms; or
h) carboxy.

In another embodiment C is of the formula (C-1):

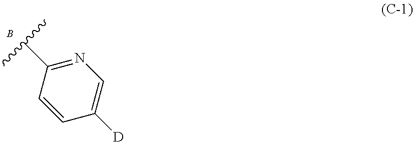

wherein D is C₆₋₁₀carboaryl, C₅₋₁₀ heteroaryl or C₅₋₁₀ heterocyclyl, which are themselves optionally substituted by ═O, halo, CN, NH₂, OH, one or more C₁₋₆ alkyl groups, which alkyl is optionally substituted by halo or C₁₋₆ alkyl sulfonyl, C₁₋₆ alkoxy which is optionally substituted by halo, C₁₋₆alkylester, C₅₋₆ heterocyclyl (with an optional methyl substituent), carboxy, CH₂-carboxy, P(═O)Me₂, tetrazolyl, pyrazolyl optionally substituted by methyl or triazolyl. In some embodiments the optional substituents are selected from ═O, CN, F, Cl, Br, methyl, ethyl, OMe, ethoxy, O—CF₃, OMe, CF₃, P(═O)Me₂ and C₁₋₂ alkyl ester. In some embodiments the optional substituents are selected from ═O, OMe, carboxy, C₁, methyl optionally substituted with —S(═O)₂CH₃, CN, phenyl substituted by F, piperazinyl substituted by methyl, CF₃, OCF₃, pyrazolyl optionally substituted by methyl, tetrazolyl or triazolyl.

In some embodiments C is of the formula (C-1):

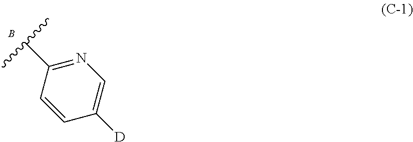

wherein D is C₆₋₁₀carboaryl, C₅₋₁₀ heteroaryl or C₅₋₁₀ heterocyclyl, each which are themselves optionally substituted by:
i) one or two ═O groups;
ii) one or two C₁₋₄ alkyl groups which can be branched;
i) OMe;
ii) piperazinyl, optionally substituted by methyl;
iii) C(═O)OH (carboxy);
iv) Cl;
v) F;
vi) phenyl, optionally substituted by one or more fluoro;
vii) CN;
viii) CF₃;
ix) O—CF₃;
x) OMe;
xi) tetrazolyl, pyrazolyl, triazolyl;
xii) NH₂;
xiii) (CH₃)₂;
xiv) pyridinyl;
xv) CH₂OH;
xvi) OH; or
xvii) P(═O)Me₂.

In some embodiments C is of the formula (C-1):

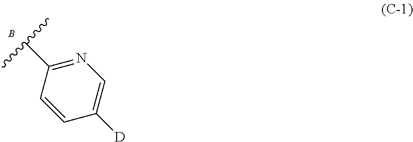

wherein D is C₆₋₁₀carboaryl, C₅₋₁₀ heteroaryl or C₅₋₁₀ heterocyclyl, each which are themselves optionally substituted by:
i) one or two ═O groups;
ii) one or two C₁₋₄ alkyl groups which can be branched, optionally substituted by S(═O)₂CH₃;
i) OMe;
ii) piperazinyl, optionally substituted by methyl;
iii) C(═O)OH (carboxy);
iv) Cl;
v) F;
vi) phenyl, optionally substituted by one or more fluoro;
vii) CN;
viii) CF₃;
ix) O—CF₃;
x) OMe;
xi) tetrazolyl, pyrazolyl optionally substituted by methyl, triazolyl;
xii) NH₂;
xiii) pyridinyl;
xiv) CH₂OH;
xv) OH; or
xvi) P(═O)Me₂.

When C is (C-1) in some embodiments D is an optionally substituted pyridin-2-one. In some embodiments the optional substituents are selected from OMe, Cl, F, methyl, trifluoromethyl, OCF₃, —C(═O)OH (carboxy), CN, pyrazolyl, triazolyl, tetrazolyl, phenyl with an optional F substituent at the para position or piperazinyl with a methyl substituent. In some embodiments the pyridine-2-one is unsubstituted. In some embodiments the optional substituents are selected from OMe, carboxy, C₁, methyl optionally substituted with —S(═O)₂CH₃, CN, phenyl substituted by F, piperazinyl substituted by methyl, CF₃, OCF₃, pyrazolyl optionally substituted by methyl, tetrazolyl or triazolyl. In some embodiments the pyridin-2-one is pyridine-2 (1H-one. When C is (C-1), in some embodiments D is an optionally substituted 6 membered heteroaryl which contains one or two N atoms, one of which is bonded to (C-1), which is substituted by ═O at the ortho position and the other optional substituents are selected from methyl, OMe, piperazine substituted by methyl, —C(═O)OH (carboxy), C₁, phenyl substituted by fluoro, CN, CF₃, F, pyrazolyl, triazolyl, tetrazolyl, or O—CF₃. In some embodiments the optional substituents are selected from OMe, —C(═O)OH (carboxy), Cl, methyl optionally substituted with —S(═O)₂CH₃, CN, phenyl substituted by F, piperazinyl substituted by methyl, CF₃, OCF₃, pyrazolyl optionally substituted by methyl, tetrazolyl or triazolyl.

In some embodiments when C is (C-1), D is an optionally substituted 6 membered heteroaryl which contains 1 or 2 N atoms, one of which is bonded to (C-1), which is substituted by ═O at the ortho position wherein the optional substituents are selected from:
i) methyl;
ii) OMe;
iii) piperazinyl substituted by methyl;
iv) C(═O)OH (carboxy);
v) Cl;
vi) F;
vii) phenyl substituted by fluoro;
viii) CN;
ix) CF₃;
x) O—CF₃;
xi) pyrazole, triazole, tetrazole.

In some embodiments when C is (C-1), D is an optionally substituted 6 membered heteroaryl which contains 1 or 2 N atoms, one of which is bonded to (C-1), which is substituted by ═O at the ortho position wherein the optional substituents are selected from:
i) methyl optionally substituted with —S(═O)₂CH₃;
ii) OMe;
iii) piperazinyl substituted by methyl;
iv) C(═O)OH (carboxy);
v) Cl;
vi) phenyl substituted by fluoro;
vii) CN;
viii) CF₃;
ix) O—CF₃;
x) pyrazole optionally substituted by methyl, triazole or tetrazole.

In further embodiments C is of the formula (C-1) and D is an optionally substituted pyridyl, phenyl or pyridazinyl optionally substituted by methyl which methyl is optionally substituted with —S(═O)₂CH₃, OMe, piperazinyl substituted by methyl, C(O)OH (carboxy), Cl, phenyl substituted by fluoro, CN, CF₃, O—CF₃, pyrazole optionally substituted by methyl, triazole or tetrazole. In some embodiments D is an optionally substituted pyridyl or pyridazinyl.

In some embodiments C is of the formula (C-1) and D is an optionally substituted phenyl or piperidyl wherein there are one or two optional substituents selected from F, OMe and CN.

In some embodiments C is of the formula (C-1) and D is an optionally substituted C₁₀ heterocyclyl wherein the optional substituent is an ═O group. In some embodiments D is an optionally substituted 1,2,3,4-tetrahydro-1,8-naphthyridine or 1,2,3,4-tetrahydroquinoline wherein the optional substituent is an ═O group. In some embodiments D is a 3,4-dihydro-1H-1,8-naphthyridin-2-one (2-oxo-3,4-dihydro-1,8-naphthyridin-1-yl) or 3,4-dihydro-1H-quinolin-2-one (2-oxo-3,4-dihydroquinolin-1-yl). In some embodiments D is an optionally substituted 1,2-dihydro-1,8-naphthyridine or 1,2-dihydroquinoline wherein the optional substituent is an ═O group. In some embodiments D is 1H-1,8-naphthyridin-2-one (2-oxo-1,8-naphthyridin-1-yl) or 1H-quinolin-2-one (2-oxo-1-quinolyl).

In another embodiment D is of the formula (D-1):

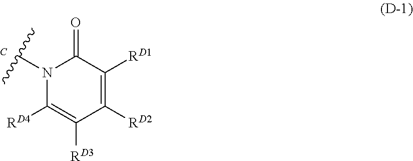

wherein one or two of R^{D1}, R^{D2}, R^{D3} and R^{D4} are selected from a C₁₋₆ alkyl optionally substituted by C₁₋₆ alkyl sulfonyl or one or more halo groups; C₁₋₆ alkoxy optionally substituted by one or more halo groups, C₅₋₆ heterocyclyl or C₅₋₆ heteroaryl with an optional methyl substituent, C(═O)OH, ═O, halo, NH₂, CN, or phenyl optionally substituted by one or more halo atoms; or wherein R^{D3} and R^{D4} form an optionally substituted 6 membered carboaromatic, heterocycle or heteroaromatic ring wherein the optional substituents are selected from OH, methyl, OMe, halo, CN, P(═O)Me₂ and C(═O)OH;

or wherein R^{D1}, R^{D2}, R^{D3} and R^{D4} are all H.

In another embodiment D is of the formula (D-1):

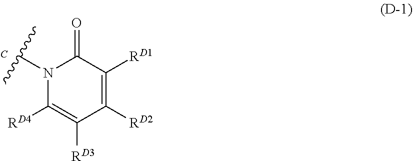

wherein one or two of R^{D1}, R^{D2}, R^{D3} and R^{D4} are selected from a C₁₋₆ alkyl optionally substituted by one or more halo groups; C₁₋₆ alkoxy optionally substituted by one or more halo groups, C₅₋₆ heterocyclyl or C₅₋₆heteroaryl with an optional methyl substituent, C(═O)OH, ═O, halo, NH₂, CN, or phenyl optionally substituted by one or more halo atoms; or

wherein R^{D3} and R^{D4} form an optionally substituted 6 membered carboaromatic, heterocycle or heteroaromatic ring wherein the optional substituents are selected from OH, methyl, OMe, halo, CN, P(═O)Me₂ and C(═O)OH;

or wherein R^{D1}, R^{D2}, R^{D3} and R^{D4} are all H.

In some embodiments D is of the formula (D-1):

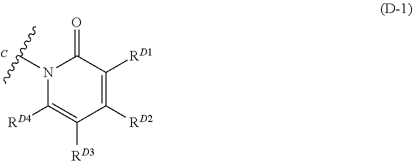

wherein one or two of R^{D1}, R^{D2}, R^{D3} and R^{D4} are selected from
i) C₁₋₆ alkyl, optionally substituted by or one or more halo groups;
ii) C₁₋₆ alkoxy, optionally substituted by one or more halo groups;
iii) C₅₋₆ heterocyclyl or C₅₋₆ heteroaryl which groups have an optional methyl substituent;
iv) C(═O)OH or CH₂—C(═O)OH;
v)═O, halo, NH₂ or CN;
vi) phenyl, optionally substituted by one or more halo atoms;

and the rest are H; or

wherein R^{D3} and R^{D4} form an optionally substituted 6 membered carboaromatic, heterocycle or heteroaromatic ring, wherein the optional substituents are selected from OH, methyl, OMe, halo and C(═O)OH;

or wherein R^{D1}, R^{D2}, R^{D3} and R^{D4} are all H.

In another embodiment one or two of R^{D1}, R^{D2}, R^{D3} and R^{D4} are selected from C₁₋₆ alkyl optionally substituted by —S(═O)₂CH₃ or one or more halo groups, C₁₋₆ alkoxy optionally substituted by one or more halo groups, C₅₋₆ heterocyclyl or C₅₋₆heteroaryl with an optional methyl substituent, C(═O)OH, halo, CN, or phenyl optionally substituted by one or more halo atoms, and the rest are H.

In some embodiments D is of the formula (D-1) and one or two of R^{D1}, R^{D2}, R^{D3} and R^{D4} are selected from:
i) methyl;
ii) OMe;
iii) piperazine substituted by methyl;
iv) C(═O)OH (carboxy);
v) Cl;
vi) phenyl substituted by fluoro;
vii) CN;
viii) CF₃;
ix) F;
x) pyrazolyl, triazolyl, tetrazolyl;
xi) O—CF₃

and the rest of R^{D1}, R^{D2}, R^{D3} and R^{D4} are H.

In some embodiments D is of the formula (D-1) and one or two of R^{D1}, R^{D2}, R^{D3} and R^{D4} are selected from:
i) methyl;
ii) OMe;
iii) piperazinyl substituted by methyl;
iv) C(═O)OH (carboxy);
v) Cl;
vi) phenyl substituted by fluoro;
vii) CN;
viii) CF₃;
ix) O—CF₃
x) F;
xi) pyrazolyl, triazolyl or tetrazolyl;
and the rest of R^{D1}, R^{D2}, R^{D3} and R^{D4} are H

In some embodiments D is of the formula (D-1) and one or two of R^{D1}, R^{D2}, R^{D3} and R^{D4} are selected from:
i) methyl optionally substituted with —S(═O)₂CH₃;
ii) OMe;
iii) piperazine substituted by methyl;
iv) C(═O)OH (carboxy);
v) Cl;
vi) phenyl substituted by fluoro;
vii) CN;
viii) CF₃;
ix) pyrazolyl optionally substituted by methyl, triazolyl, tetrazolyl;
x) O—CF₃

and the rest of R^{D1}, R^{D2}, R^{D3} and R^{D4} are H.

In another embodiment one or two of R^{D1}, R^{D2}, R^{D3} and R^{D4} are selected from methyl, —OMe, halo, —C(═O)OH, CN, CF₃, —OCF₃, —OCHF₂ and the rest of R^{D1}, R^{D2}, R^{D3} and R^{D4} are H. In another embodiment one or two of R^{D1}, R^{D2}, R^{D3} and R^{D4} are selected from methyl optionally substituted with —S(═O)₂CH₃, —OMe, halo, —C(═O)OH, CN, CF₃, —OCF₃, pyrazolyl optionally substituted by methyl, triazolyl, tetrazolyl, phenyl substituted by fluoro, piperazine substituted by methyl and the rest of R^{D1}, R^{D2}, R^{D3} and R^{D4} are H.

In one embodiment all of R^{D1}, R^{D2}, R^{D3} and R^{D4} are H.

In some embodiments R^{D3} is selected from the group consisting of: H; optionally substituted phenyl, wherein the optional substituent is halo, methyl, OMe, C(═O)OH, Cl, CN; or piperazinyl optionally substituted by methyl; or pyrazolyl, triazolyl, or tetrazolyl, and wherein R^{D1}, R^{D2} and R^{D4} are all H. In another embodiment R^{D3} is selected from the group consisting of: H; optionally substituted phenyl, wherein the optional substituent is halo, methyl optionally substituted by —S(═O)₂CH₃, OMe, C(═O)OH, Cl, CN; or piperazinyl optionally substituted by methyl; or pyrazolyl optionally substituted by methyl, triazolyl, or tetrazolyl, and wherein R^{D1}, R^{D2} and R^{D4} are all H. In another embodiment R^{D3} is selected from H, optionally substituted phenyl (wherein the optional substituent is halo), methyl, OMe, —C(═O)OH, —OCHF₂, Cl, CN and piperazinyl optionally substituted by methyl wherein R^{D1}, R^{D2} and R^{D4} are all H. In another embodiment R^{D3} is selected from H, methyl optionally substituted by —S(═O)₂CH₃, OMe, —C(═O)OH, Cl, CN, phenyl optionally substituted with F, piperazine substituted by methyl, pyrazolyl optionally substituted by methyl, triazolyl or tetrazolyl.

In other embodiments R^{D1} is selected from H, methyl, OMe, Cl, CF₃, OCF₃, OCHF₂ and CN and R^{D2}, R^{D3} and R^{D4} are all H. In another embodiment R^{D1} is selected from H, methyl, OMe, Cl, F, CF₃, OCF₃, and CN, and wherein R^{D2}, R^{D3} and R^{D4} are all H. In other embodiments R^{D1} is selected from H, methyl, OMe, Cl, CF₃, OCF₃, pyrazolyl optionally substituted by methyl, triazolyl and CN and R^{D2}, R^{D3} and R^{D4} are all H.

In another embodiment R^{D3} and R^{D4} form an optionally substituted 6 membered carboaromatic, heterocycle or heteroaromatic ring wherein the optional substituents are selected from OH, CN, P(═O)Me₂, methyl, OMe, halo and C(═O)OH.

In some embodiments R^{D3} and R^{D4} form an optionally substituted benzene ring or an optionally substituted pyridine ring.

In some embodiments R^{D3} and R^{D4} form an unsubstituted benzene ring or an unsubstituted pyridine ring.

In another embodiment D is of the formula (D-2):

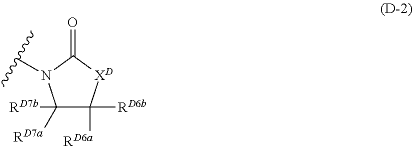

Wherein X^{D} is NR^{D5a} or CR^{D5a}R^{D5b};

R^{D5a} is selected from H or methyl;

either R^{D5b} and R^{D6b} are both H or together they are —CH₂—;

R^{D6a} is selected from H, ═O, methyl, —CH₂OH or —C(═O)OH, wherein when R^{D6a} is ═O, R^{D6b} is absent;

R^{D7a} is selected from H, ═O, methyl, —CH₂OH or —C(═O)OH;

R^{D7b} is H, wherein when R^{D7a} is ═O, R^{D7b} is absent;

or wherein R^{D6a} and R^{D7a} together form a benzene ring or a C heteroaromatic ring which is optionally substituted by CN, P(═O)Me₂ or carboxy and R^{D6b} and R^{D7b} are absent.

In some embodiments C is of the formula (C-1) and D is of the formula (D-2) and X^{D} is N and R^{D5a} is methyl.

In some embodiments C is of the formula (C-1) and D is of the formula (D-2) and R^{D6a} and R^{D6b} both H and R^{D7a} is selected from ═O, —CH₂OH or —C(═O)OH and R^{D7b} is H or when R^{D7a} is ═O R^{D7b} is absent.

In some embodiments C is of the formula (C-1) and D is of the formula (D-2) and X^{D} is N and R^{D5a} is methyl wherein R^{D7a} is selected from H and ═O and R^{D6a} is H and ═O, wherein when R^{D7a} is ═O, R^{D6a} and R^{D6b} are H and wherein R^{D6a} is ═O, R^{D7a} is H and R^{D6b} is absent and R^{D7b} is H or when R^{D7a} is ═O, R^{D7b} is absent.

In some embodiments C is of the formula (C-1) and D is of the formula (D-2), R^{D7a} is ═O, X^{D} is O and R^{D5b} and R^{D6b} together are —CH₂— and R^{D6a} and R^{D5a} are methyl.

In another embodiment R^{D6a} and R^{D7a} together form a benzene ring or a C₆ heteroaromatic ring which is optionally substituted by CN, P(═O)Me₂ or —C(═O)OH, and R^{D6b} and R^{D7b} are absent. In some embodiments R^{D6a} and R^{D7a} together form a benzene ring or a pyridyl ring which is optionally substituted with CN.

In some embodiments R^{D6a} and R^{D7a} form an unsubstituted benzene ring and R^{D6b} and R^{D7b} are absent. In some embodiments R^{D6a} and R^{D7a} form an unsubstituted pyridine ring and R^{D6b} and R^{D7b} are absent.

In some embodiments X^{D} is N and R^{D5a} is H or methyl.

In some embodiments X^{D} is C and R^{D5a} is H or methyl and R^{D5b} is H. In further embodiments both R^{D5a} and R^{D5b} are H.

In some embodiments R^{D7a} is carboxy, —CH₂OH or ═O. In some embodiments R^{D7a} is ═O. R^{D7b} is H, or when R^{D7a} is ═O, R^{D7b} is absent.

In some embodiments R^{D6a} is H or ═O. In some embodiments R^{D6a} is ═O and R^{D6b} is absent.

In some embodiments where R^{D7a} is ═O, R^{D6a} and R^{D6b} are H and R^{D7b} is absent and where R^{D6a} is ═O, R^{D7a} and R^{D7b} are H and R^{D6a} is absent.

In some embodiments X^{D} is N and R^{D5a} is H or methyl, R^{D6b} is H and R^{D6a} and R^{D7a} together form a pyridine ring or a benzene ring which is optionally substituted by —C(═O)OH or CN or P(═O)Me₂ and R^{D6b} and R^{D7b} are absent.

In some embodiments X^{D} is CR^{D5a}R^{D5b}, R^{D5a} and R^{D6a} are methyl, R^{D7a} is ═O and R^{D7b} is absent and R^{D6b} and R^{D5b} together form a 3 membered ring.

In some embodiments D is (7-oxo-2,3,3a,4,5,7a-hexahydrofuro[2,3-c]pyridin-6-yl). In some embodiments D is [(1R,5S)-1,5-dimethyl-2,4-dioxo-3-azabicyclo[3.1.0]hexan-3-yl].

In some embodiments D is selected from the following table:

| Name | Structure |
|---|---|
| 2-oxo-1-pyridyl | 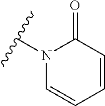 [SMILES: *n1ccccc1=O] |
| 3-methyl-2-oxo-1- pyridyl | 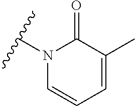 [SMILES: *n1cccc(C)c1=O] |
| 3-methoxy-2- oxo-1-pyridyl | 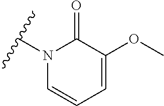 [SMILES: *n1cccc(OC)c1=O] |
| 5-methoxy-2-oxo- 1-pyridyl | 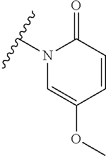 [SMILES: *n1cc(OC)ccc1=O] |
| 5-chloro-2-oxo- 1-pyridyl | 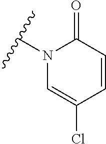 [SMILES: *n1cc(Cl)ccc1=O] |
| 2-oxo-1,8- naphthyridin-1-yl | 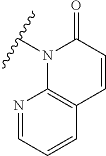 [SMILES: *n1c(=O)ccc2cccnc21] |
| 2-oxo-1-quinolyl | 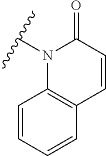 [SMILES: *n1c(=O)ccc2ccccc21] |
| 3-methyl-2-oxo- benzimidazol-1-yl | 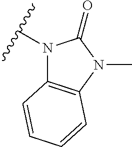 [SMILES: *n1c(=O)n(C)c2ccccc21] |
| 3-methyl-2,4- dioxo- imidazolidin-1-yl | 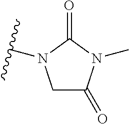 [SMILES: *N1CC(=O)N(C)C1=O] |
| 3-methyl-2,5- dioxo-imidazolidin- 1-yl | 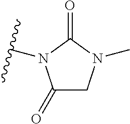 [SMILES: *N1C(=O)CN(C)C1=O] |
| 3-chloro-2-oxo- 1-pyridyl | 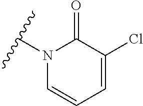 [SMILES: *n1cccc(Cl)c1=O] |
| 3-cyano-2-oxo-1- pyridyl | 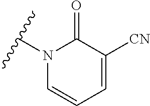 [SMILES: *n1cccc(C#N)c1=O] |
| 5-cyano-2-oxo- 1-pyridyl | 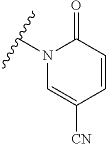 [SMILES: *n1cc(C#N)ccc1=O] |
| 3-fluoro-2- oxopyridin-1(2H)- yl | 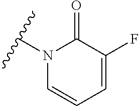 [SMILES: *n1cccc(F)c1=O] |
| 5-methyl-2-oxo- 1-pyridyl | 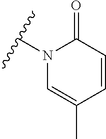 [SMILES: *n1cc(C)ccc1=O] |
| 5-carboxy-2-oxo- 1-pyridyl | 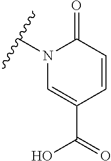 [SMILES: *n1cc(C(=O)O)ccc1=O] |
| 2-oxo-5-(1H- pyrazol-4-yl)-1- pyridyl | 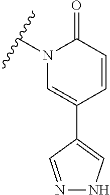 [SMILES: *n1cc(-c2cn[nH]c2)ccc1=O] |
| 2-oxo-5-(1H- triazol-4-yl)-1- pyridyl | 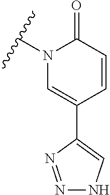 [SMILES: *n1cc(-c2c[nH]nn2)ccc1=O] |
| 5-cyano-3- methyl-2-oxo- benzimidazol-1- yl | 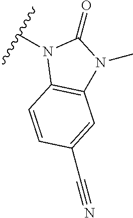 [SMILES: *n1c(=O)n(C)c2cc(C#N)ccc21] |
| 5-carboxy-3- methyl-2-oxo- benzimidazol-1-yl | 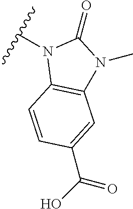 [SMILES: *n1c(=O)n(C)c2cc(C(=O)O)ccc21] |
| 6-carboxy-3- methyl-2-oxo- benzimidazol-1- yl | 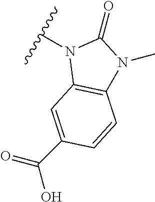 [SMILES: *n1c(=O)n(C)c2ccc(C(=O)O)cc21] |
| (6- dimethylphosphor- yl-3-methyl-2-oxo- benzimidazol-1-yl) | 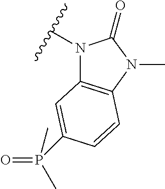 [SMILES: *n1c(=O)n(C)c2ccc(P(C)(C)=O)cc21] |
| 2-oxo-3H- benzimidazol-1- yl | 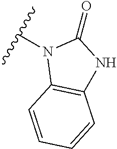 [SMILES: *n1c(=O)[nH]c2ccccc21] |
| 2-oxo-1H- imidazo[4,5- b]pyridin-3-yl | 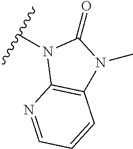 [SMILES: *n1c(=O)n(C)c2cccnc21] |
| 6-oxopyridazin- 1-yl | 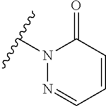 [SMILES: *n1ncccc1=O] |
| 6-oxopyrimidin-1- yl | 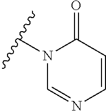 [SMILES: *n1cnccc1=O] |
| 1-methyl-2-oxo- 3-pyridyl | 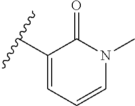 [SMILES: *c1cccn(C)c1=O] |
| 2-oxo-3- (trifluoromethyl)-1- pyridyl | 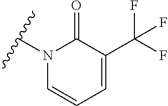 [SMILES: *n1cccc(C(F)(F)F)c1=O] |
| 2-oxo-3- (trifluoromethoxy)- 1-pyridyl | 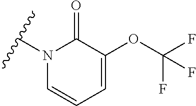 [SMILES: *n1cccc(OC(F)(F)F)c1=O] |
| 2-oxo-5-(1H- tetrazol-5-yl)-1- pyridyl | 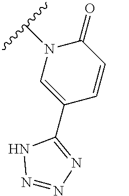 |

In some embodiments D is selected from the following table

| | |
|---|---|
| [3-(1-methylpyrazol-4- yl)-2-oxo-1-pyridyl] | 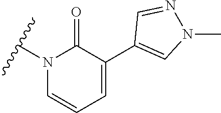 [SMILES: *n1cccc(-c2cnn(C)c2)c1=O] |
| [2-oxo-3-(1H- pyrazol-4-yl)-1- pyridyl] | 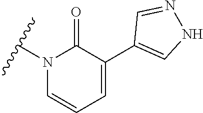 [SMILES: *n1cccc(-c2cn[nH]c2)c1=O] |
| [2-oxo-3-(1H-triazol-4- yl)-1-pyridyl] | 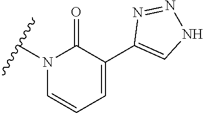 [SMILES: *n1cccc(-c2c[nH]nn2)c1=O] |
| [5-(1- methylpyrazol-4-yl)- 2-oxo-1-pyridyl] | 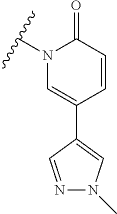 [SMILES: *n1cc(-c2cnn(C)c2)ccc1=O] |
| (6-cyano-1-methyl-2- oxo-imidazo[4,5- b]pyridin-3-yl) | 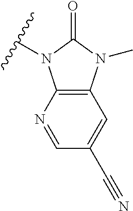 [SMILES: *n1c(=O)n(C)c2cc(C#N)cnc21] |
| (5-methoxy-6-oxo- pyridazin-1-yl) | 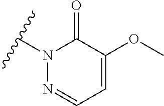 [SMILES: *n1nccc(OC)c1=O] |
| (5-methyl-6-oxo- pyridazin-1-yl) | 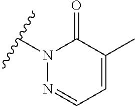 [SMILES: *n1nccc(C)c1=O] |
| (3-methyl-6-oxo- pyridazin-1-yl) | 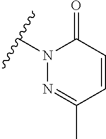 [SMILES: *n1nc(C)ccc1=O] |
| (5-chloro-6-oxo- pyridazin-1-yl) | 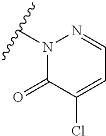 [SMILES: *n1nccc(Cl)c1=O] |
| (3-chloro-6-oxo- pyridazin-1-yl) | 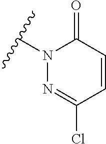 [SMILES: *n1nc(Cl)ccc1=O] |
| [5- (methylsulfonylmethyl)- 2-oxo-1-pyridyl] | 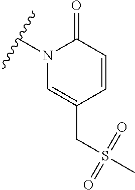 [SMILES: *n1cc(CS(C)(=O)=O)ccc1=O] |
| (2-oxo-3,4- dihydroquinolin-1- yl) | 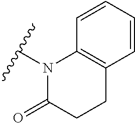 [SMILES: *N1C(=O)CCc2ccccc21] |
| (5-cyano-6-oxo- pyridazin-1-yl) | 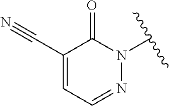 [SMILES: *n1nccc(C#N)c1=O] |
| [5- (difluoromethoxy)- 6-oxo-pyridazin-1- yl] | 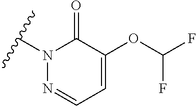 [SMILES: *n1nccc(OC(F)F)c1=O] |
| (2-oxo-3,4-dihydro-1,8- naphthyridin-1-yl) | 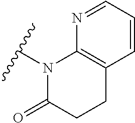 [SMILES: *N1C(=O)CCc2cccnc21] |

In some embodiments D is selected from the following table

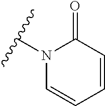 [SMILES: *n1ccccc1=O]

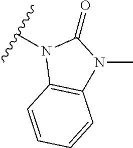 [SMILES: *n1c(=O)n(C)c2ccccc21]

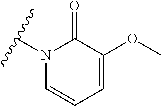 [SMILES: *n1cccc(OC)c1=O]

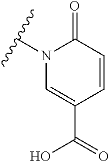 [SMILES: *n1cc(C(=O)O)ccc1=O]

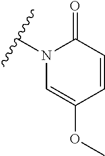 [SMILES: *n1cc(OC)ccc1=O]

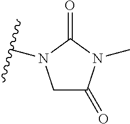 [SMILES: *N1CC(=O)N(C)C1=O]

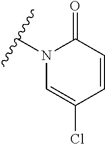 [SMILES: *n1cc(Cl)ccc1=O]

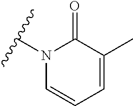 [SMILES: *n1cccc(C)c1=O]

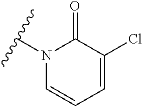 [SMILES: *n1cccc(Cl)c1=O]

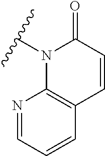 [SMILES: *n1c(=O)ccc2cccnc21]

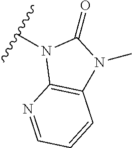 [SMILES: *n1c(=O)n(C)c2cccnc21]

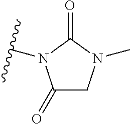 [SMILES: *N1C(=O)CN(C)C1=O]

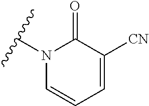 [SMILES: *n1cccc(C#N)c1=O]

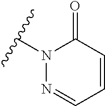 [SMILES: *n1ncccc1=O]

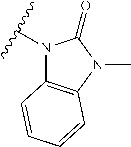 [SMILES: *n1c(=O)n(C)c2ccccc21]

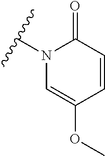 [SMILES: *n1cc(OC)ccc1=O]

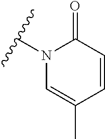 [SMILES: *n1cc(C)ccc1=O]

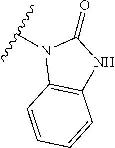 [SMILES: *n1c(=O)[nH]c2ccccc21]

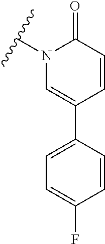 [SMILES: *n1cc(-c2ccc(F)cc2)ccc1=O]

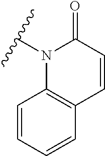 [SMILES: *n1c(=O)ccc2ccccc21]

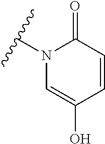 [SMILES: *n1cc(O)ccc1=O]

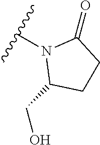

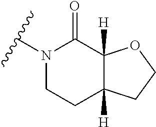 [SMILES: *N1CC[C@H]2CCO[C@H]2C1=O]

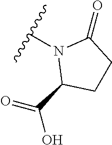 [SMILES: *N1C(=O)CC[C@H]1C(=O)O]

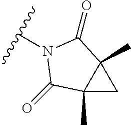 [SMILES: *N1C(=O)[C@]2(C)C[C@]2(C)C1=O]

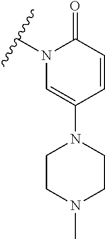 [SMILES: *n1cc(N2CCN(C)CC2)ccc1=O]

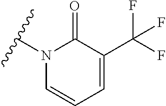 [SMILES: *n1cccc(C(F)(F)F)c1=O]

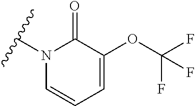 [SMILES: *n1cccc(OC(F)(F)F)c1=O]

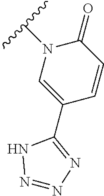 [SMILES: *n1cc(-c2nnn[nH]2)ccc1=O]

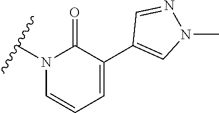 [SMILES: *n1cccc(-c2cnn(C)c2)c1=O]

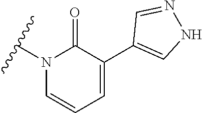 [SMILES: *n1cccc(-c2cn[nH]c2)c1=O]

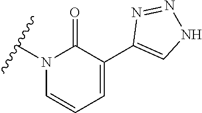 [SMILES: *n1cccc(-c2c[nH]nn2)c1=O]

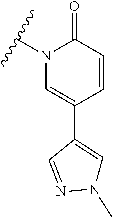 [SMILES: *n1cc(-c2cnn(C)c2)ccc1=O]

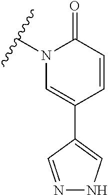 [SMILES: *n1cc(-c2cn[nH]c2)ccc1=O]

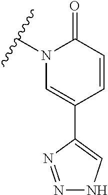 [SMILES: *n1cc(-c2c[nH]nn2)ccc1=O]

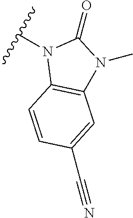 [SMILES: *n1c(=O)n(C)c2cc(C#N)ccc21]

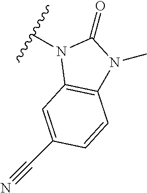 [SMILES: *n1c(=O)n(C)c2ccc(C#N)cc21]

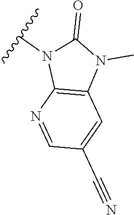 [SMILES: *n1c(=O)n(C)c2cc(C#N)cnc21]

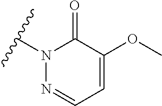 [SMILES: *n1nccc(OC)c1=O]

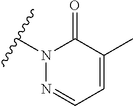 [SMILES: *n1nccc(C)c1=O]

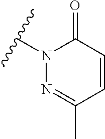 [SMILES: *n1nc(C)ccc1=O]

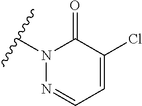 [SMILES: *n1nccc(Cl)c1=O]

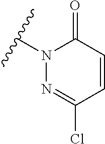 [SMILES: *n1nc(Cl)ccc1=O]

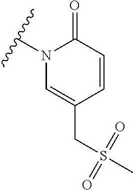 [SMILES: *n1cc(CS(C)(=O)=O)ccc1=O]

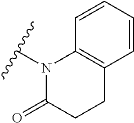 [SMILES: *N1C(=O)CCc2ccccc21]

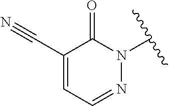 [SMILES: *n1nccc(C#N)c1=O]

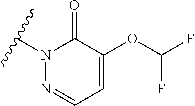 [SMILES: *n1nccc(OC(F)F)c1=O]

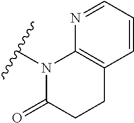 [SMILES: *N1C(=O)CCc2cccnc21]

In another embodiment C has the formula (C-2):

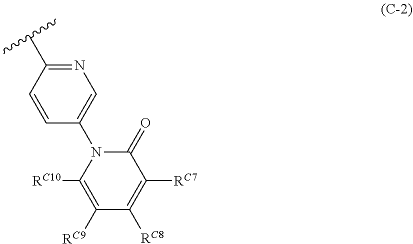

wherein one or two of R^{C7}, R^{C8}, R^{C9} and R^{C10} are selected from methyl optionally substituted with —S(═O)₂CH₃, OMe, halo, —C(═O)OH, piperazine optionally substituted by methyl, optionally substituted phenyl (wherein the optional substituent is methyl or halo), CN, CF₃, —O—CF₃, tetrazolyl, pyrazolyl optionally substituted with methyl, or triazolyl and the rest of R^{C7}, R^{C8}, R^{C9} and R^{C10} are H; or R^{C9} and R^{C10} form an optionally substituted 6 membered carboaromatic, heterocycle or heteroaromatic ring wherein the optional substituents are selected from OH, methyl, OMe, halo, CN, P(═O)Me₂ or —C(═O)OH; or wherein R^{C7}, R^{C8}, R^{C9} and R^{C10} are all H.

In some embodiments one of R^{C7}, R^{C8}, R^{C9} and R^{C10} are selected from the group consisting of: methyl; OMe; piperazinyl optionally substituted by methyl; —C(═O)OH (carboxy); Cl; F; pyrazolyl; triazolyl; tetrazole; optionally substituted phenyl (wherein the optional substituent is methyl or halo); CN; CF₃; O—CF₃; and the rest of R^{C7}, R^{C8}, R^{C9} and R^{C10} are H; or R^{C9} and R^{C10} form a benzene or 6 membered heteroaromatic ring, and R^{C7} and R^{C8} are both H; or wherein R^{C7}, R^{C8}, R^{C9} and R^{C10} are all H. In other embodiments one of R^{C7}, R^{C8}, R^{C9} and R^{C10} are selected from the group consisting of: methyl optionally substituted with —S(═O)₂CH₃; OMe; piperazinyl optionally substituted by methyl; —C(═O)OH (carboxy); Cl; F; pyrazolyl optionally substituted with methyl; triazolyl; tetrazole; optionally substituted phenyl (wherein the optional substituent is methyl or halo); CN; CF₃; O—CF₃; and the rest of R^{C7}, R^{C8}, R^{C9} and R^{C10} are H; or R^{C9} and R^{C10} form a benzene or 6 membered heteroaromatic ring, and R^{C7} and R^{C8} are both H; or wherein R^{C7}, R^{C8}, R^{C9} and R^{C10} are all H

In some embodiments one or two of R^{C7}, R^{C8}, R^{C9} and R^{C10} are selected from methyl optionally substituted with —S(═O)₂CH₃ OMe, halo, C(═O)OH, piperazine optionally substituted by methyl, optionally substituted phenyl (wherein the optional substituent is methyl or halo), CN, CF₃, —O—CF₃, tetrazolyl, pyrazolyl optionally substituted with methyl, or triazolyl and the rest of R^{C7}, R^{C8}, R^{C9} and R^{C10} are H; or R^{C9} and R^{C10} form an optionally substituted 6 membered carboaromatic, heterocycle or heteroaromatic ring wherein the optional substituents are selected from OH, methyl, OMe, halo, CN, P(═O)Me₂ or —C(═O)OH; or wherein R^{C7}, R^{C8}, R^{C9} and R^{C10} are all H.

In one embodiment C has the formula (C-1) and all of R^{C7}, R^{C8}, R^{C9} and R^{C10} are H.

In another embodiment one or two of R^{C7}, R^{C8}, R^{C9} and R^{C10} are selected from methyl, Cl, OMe, phenyl substituted with F at para position, —C(═O)OH, CN, OCF₃, CF₃, F, pyrazolyl, triazolyl, tetrazolyl, piperazinyl substituted by methyl and the rest of R^{C7}, R^{C8}, R^{C9} and R^{C10} are H. In another embodiment one or two of R^{C7}, R^{C8}, R^{C9} and R^{C10} are selected from methyl optionally substituted with —S(═O)₂CH₃, Cl, OMe, phenyl substituted with F at the para position, —C(═O)OH, CN, OCF₃, CF₃, pyrazolyl optionally substituted by methyl, triazolyl, tetrazolyl, piperazinyl substituted by methyl and the rest of R^{C7}, R^{C8}, R^{C9} and R^{C10} are H.

In another embodiment one of R^{C7} and R^{C9} are independently selected from methyl, —OMe, CI, —C(═O)OH, piperazinyl optionally substituted by methyl, optionally substituted phenyl (wherein the optional substituent is F), CN, CF₃, —O—CF₃, F, pyrazolyl, triazolyl or tetrazolyl and the other and is H and R^{C10} and R^{C8} are H. In another embodiment one of R^{C7} and R^{C9} are independently selected from methyl optionally substituted with —S(═O)₂CH₃, Cl, OMe, phenyl substituted with F at the para position, —C(═O)OH, CN, OCF₃, CF₃, pyrazolyl optionally substituted by methyl, triazolyl, tetrazolyl, piperazinyl substituted by methyl and R^{C8} and R^{C10} are H.

In another embodiment R^{C9} is selected from H, optionally substituted phenyl (wherein the optional substituent is halo), methyl, OMe, C(═O)OH, Cl, CN, pyrazolyl, triazolyl, tetrazolyl, and piperazinyl optionally substituted with methyl and R^{C7}, R^{C8} and R^{C10} are all H. In another embodiment R^{C9} is selected from H, methyl optionally substituted with —S(═O)₂CH₃, OMe, —C(═O)OH, Cl, CN, phenyl optionally substituted with F, piperazine substituted by methyl, pyrazolyl optionally substituted by methyl, triazolyl or tetrazolyl.

In other embodiments R^{C7} is selected from H, methyl, OMe, Cl, F, CF₃, —OCF₃ and CN and R^{C8}, R^{C9} and R^{C10} are all H. In other embodiments R^{C7} is selected from H, methyl, OMe, Cl, CF₃, OCF₃, pyrazolyl optionally substituted by methyl, triazolyl and CN and R^{C8}, R^{C9} and R^{C10} are all H.

In some embodiments:
a) all of R^{C7}, R^{C8}, R^{C9} and R^{C10} are H; or
b) R^{C9} is selected from H, optionally substituted phenyl (wherein the optional substituent is F), methyl optionally substituted with —S(═O)₂CH₃, OMe, C(═O)OH, Cl, pyrazolyl optionally substituted by methyl, triazolyl, tetrazolyl, CN and piperazinyl optionally substituted by methyl, and R^{C7}, R^{C8} and R^{C10} are all H; or
c) R^{C7} is selected from H, methyl, OMe, O—CF₃, CF₃, Cl, pyrazolyl optionally substituted by methyl, triazolyl and CN and R^{C8}, R^{C9} and R^{C10} are all H.

In other embodiments:
a) all of R^{C7}, R^{C8}, R^{C9} and R^{C10} are H; or
b) R^{C9} is selected from H, optionally substituted phenyl (wherein the optional substituent is F), methyl, OMe, —C(═O)OH, Cl, F, pyrazolyl, triazolyl, tetrazolyl, CN and piperazinyl optionally substituted by methyl, and R^{C7}, R^{C8} and R^{C10} are all H; or
c) R^{C7} is selected from H, methyl, OMe, O—CF₃, CF₃, Cl, F, pyrazolyl and CN and R^{C8}, R^{C9} and R^{C10} are all H.

In another embodiment C is selected from the group consisting of the groups 1-48 listed in the following table:

| No. | Structure |
|---|---|
| 1 | 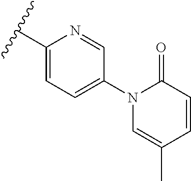 [SMILES: *c1ccc(-n2cc(C)ccc2=O)cn1] |
| 2 | 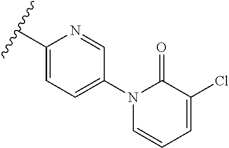 [SMILES: *c1ccc(-n2cccc(Cl)c2=O)cn1] |
| 3 | 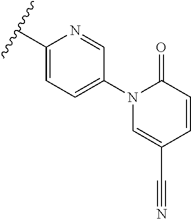 [SMILES: *c1ccc(-n2cc(C#N)ccc2=O)cn1] |
| 4 | 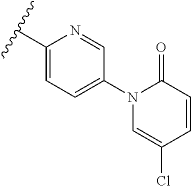 [SMILES: *c1ccc(-n2cc(Cl)ccc2=O)cn1] |
| 5 | 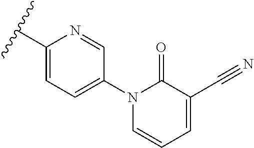 [SMILES: *c1ccc(-n2cccc(C#N)c2=O)cn1] |
| 6 | 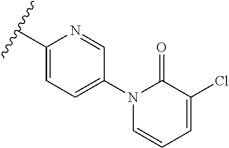 [SMILES: *c1ccc(-n2cccc(Cl)c2=O)cn1] |
| 7 | 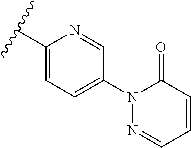 [SMILES: *c1ccc(-n2ncccc2=O)cn1] |
| 8 | 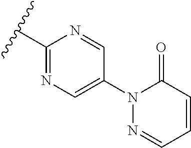 [SMILES: *c1ncc(-n2ncccc2=O)cn1] |
| 9 | 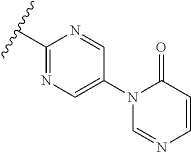 [SMILES: *c1ncc(-n2cnccc2=O)cn1] |
| 10 | 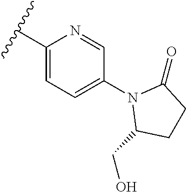 |
| 11 | 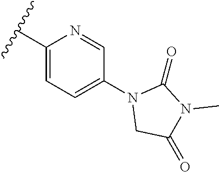 [SMILES: *c1ccc(N2CC(=O)N(C)C2=O)cn1] |
| 12 | 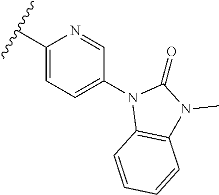 [SMILES: *c1ccc(-n2c(=O)n(C)c3ccccc32)cn1] |
| 13 | 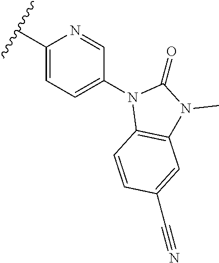 [SMILES: *c1ccc(-n2c(=O)n(C)c3cc(C#N)ccc32)cn1] |
| 14 | 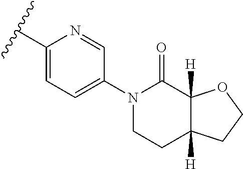 [SMILES: *c1ccc(N2CC[C@H]3CCO[C@H]3C2=O)cn1] |
| 15 | 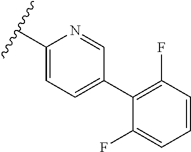 [SMILES: *c1ccc(-c2c(F)cccc2F)cn1] |
| 16 | 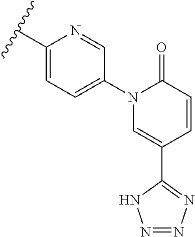 [SMILES: *c1ccc(-n2cc(-c3nnn[nH]3)ccc2=O)cn1] |
| 17 | 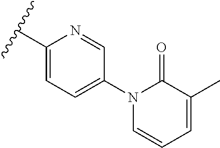 [SMILES: *c1ccc(-n2cccc(C)c2=O)cn1] |
| 18 | 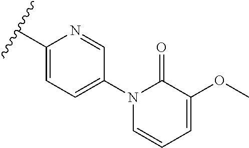 [SMILES: *c1ccc(-n2cccc(OC)c2=O)cn1] |
| 19 | 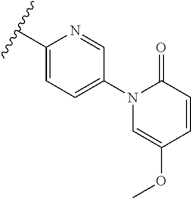 [SMILES: *c1ccc(-n2cc(OC)ccc2=O)cn1] |
| 20 | 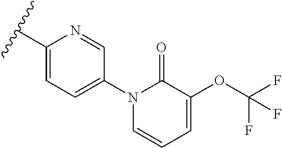 [SMILES: *c1ccc(-n2cccc(OC(F)(F)F)c2=O)cn1] |
| 21 | 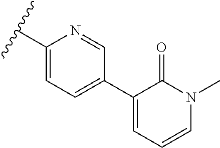 [SMILES: *c1ccc(-c2cccn(C)c2=O)cn1] |
| 22 | 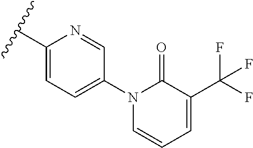 [SMILES: *c1ccc(-n2cccc(C(F)(F)F)c2=O)cn1] |
| 23 | 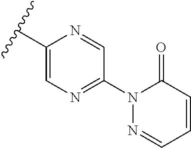 [SMILES: *c1cnc(-n2ncccc2=O)cn1] |
| 24 | 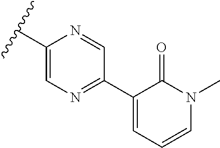 [SMILES: *c1cnc(-c2cccn(C)c2=O)cn1] |
| 25 | 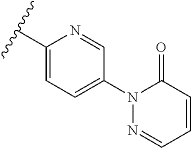 [SMILES: *c1ccc(-n2ncccc2=O)cn1] |
| 26 | 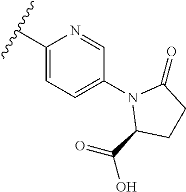 [SMILES: *c1ccc(N2C(=O)CC[C@H]2C(=O)O)cn1] |
| 27 | 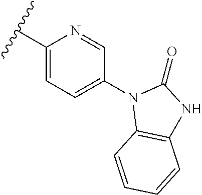 [SMILES: *c1ccc(-n2c(=O)[nH]c3ccccc32)cn1] |
| 28 | 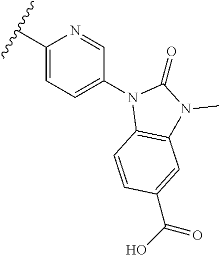 [SMILES: *c1ccc(-n2c(=O)n(C)c3cc(C(=O)O)ccc32)cn1] |
| 29 | 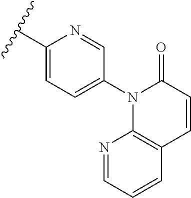 [SMILES: *c1ccc(-n2c(=O)ccc3cccnc32)cn1] |
| 30 | 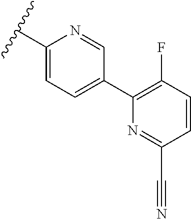 [SMILES: *c1ccc(-c2nc(C#N)ccc2F)cn1] |
| 31 | 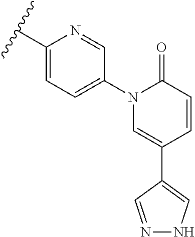 [SMILES: *c1ccc(-n2cc(-c3cn[nH]c3)ccc2=O)cn1] |
| 32 | 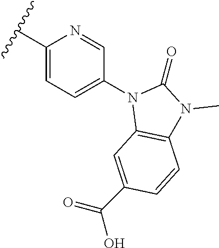 [SMILES: *c1ccc(-n2c(=O)n(C)c3ccc(C(=O)O)cc32)cn1] |
| 33 | 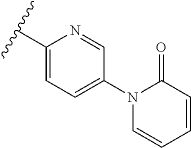 [SMILES: *c1ccc(-n2ccccc2=O)cn1] |
| 34 | 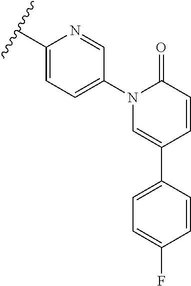 [SMILES: *c1ccc(-n2cc(-c3ccc(F)cc3)ccc2=O)cn1] |
| 35 | 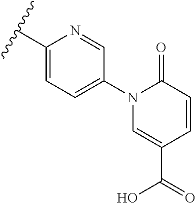 [SMILES: *c1ccc(-n2cc(C(=O)O)ccc2=O)cn1] |
| 36 | 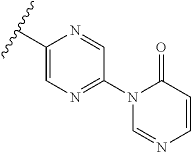 [SMILES: *c1cnc(-n2cnccc2=O)cn1] |
| 37 | 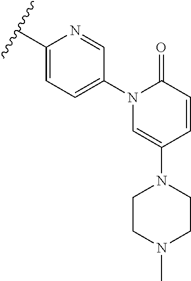 [SMILES: *c1ccc(-n2cc(N3CCN(C)CC3)ccc2=O)cn1] |
| 38 | 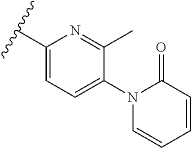 [SMILES: *c1ccc(-n2ccccc2=O)c(C)n1] |
| 39 | 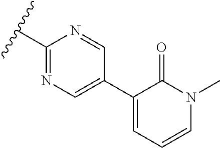 [SMILES: *c1ncc(-c2cccn(C)c2=O)cn1] |
| 40 | 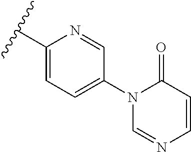 [SMILES: *c1ccc(-n2cnccc2=O)cn1] |
| 41 | 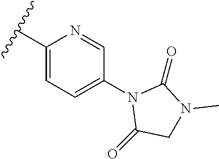 [SMILES: *c1ccc(N2C(=O)CN(C)C2=O)cn1] |
| 42 | 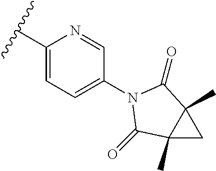 [SMILES: *c1ccc(N2C(=O)[C@]3(C)C[C@]3(C)C2=O)cn1] |
| 43 | 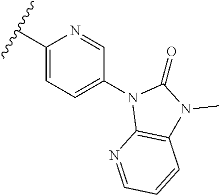 [SMILES: *c1ccc(-n2c(=O)n(C)c3cccnc32)cn1] |
| 44 | 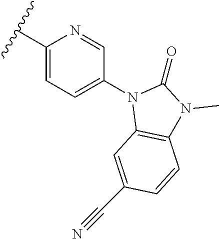 [SMILES: *c1ccc(-n2c(=O)n(C)c3ccc(C#N)cc32)cn1] |
| 45 | 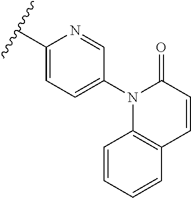 [SMILES: *c1ccc(-n2c(=O)ccc3ccccc32)cn1] |
| 46 | 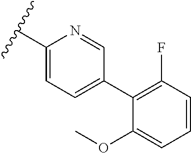 [SMILES: *c1ccc(-c2c(F)cccc2OC)cn1] |
| 47 | 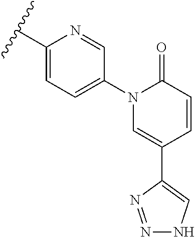 [SMILES: *c1ccc(-n2cc(-c3c[nH]nn3)ccc2=O)cn1] |
| 48 | 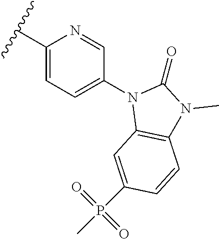 |

In another embodiment C is selected from the group consisting of the groups listed in the following table:

Structure

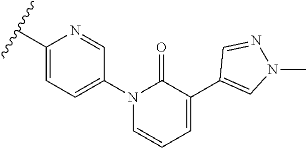 [SMILES: *c1ccc(-n2cccc(-c3cnn(C)c3)c2=O)cn1]

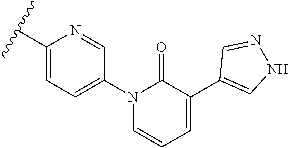 [SMILES: *c1ccc(-n2cccc(-c3cn[nH]c3)c2=O)cn1]

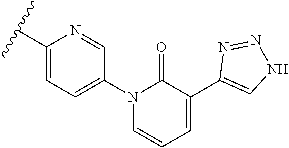 [SMILES: *c1ccc(-n2cccc(-c3c[nH]nn3)c2=O)cn1]

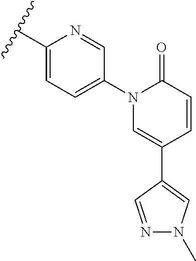 [SMILES: *c1ccc(-n2cc(-c3cnn(C)c3)ccc2=O)cn1]

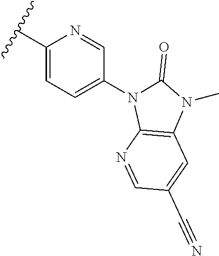 [SMILES: *c1ccc(-n2c(=O)n(C)c3cc(C#N)cnc32)cn1]

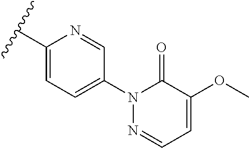 [SMILES: *c1ccc(-n2nccc(OC)c2=O)cn1]

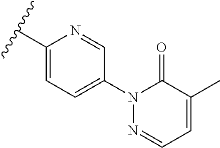 [SMILES: *c1ccc(-n2nccc(C)c2=O)cn1]

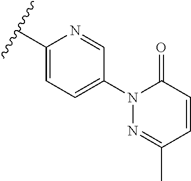 [SMILES: *c1ccc(-n2nc(C)ccc2=O)cn1]

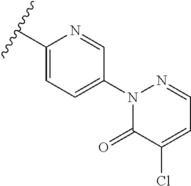 [SMILES: *c1ccc(-n2nccc(Cl)c2=O)cn1]

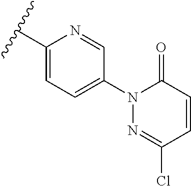 [SMILES: *c1ccc(-n2nc(Cl)ccc2=O)cn1]

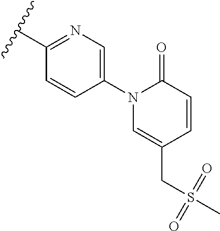 [SMILES: *c1ccc(-n2cc(CS(C)(=O)=O)ccc2=O)cn1]

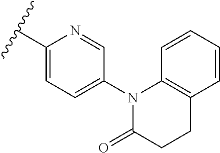 [SMILES: *c1ccc(N2C(=O)CCc3ccccc32)cn1]

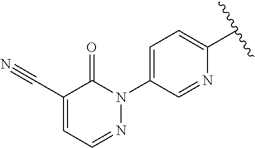 [SMILES: *c1ccc(-n2nccc(C#N)c2=O)cn1]

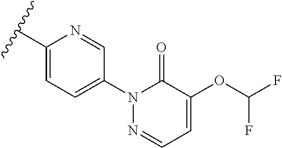 [SMILES: *c1ccc(-n2nccc(OC(F)F)c2=O)cn1]

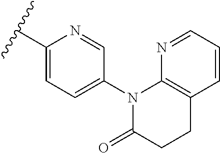 [SMILES: *c1ccc(N2C(=O)CCc3cccnc32)cn1]

In another embodiment C is selected from the group consisting of the groups listed in the following table:

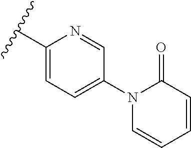 [SMILES: *c1ccc(-n2ccccc2=O)cn1]

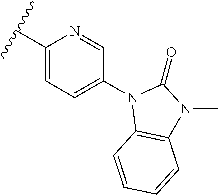 [SMILES: *c1ccc(-n2c(=O)n(C)c3ccccc32)cn1]

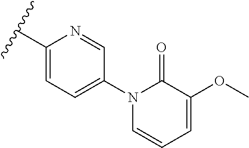 [SMILES: *c1ccc(-n2cccc(OC)c2=O)cn1]

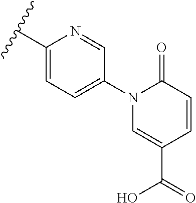 [SMILES: *c1ccc(-n2cc(C(=O)O)ccc2=O)cn1]

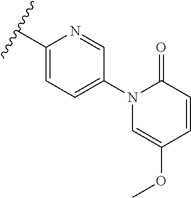 [SMILES: *c1ccc(-n2cc(OC)ccc2=O)cn1]

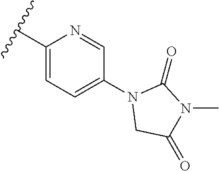 [SMILES: *c1ccc(N2CC(=O)N(C)C2=O)cn1]

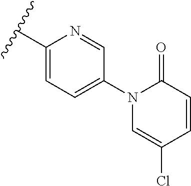 [SMILES: *c1ccc(-n2cc(Cl)ccc2=O)cn1]

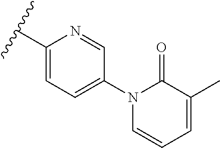 [SMILES: *c1ccc(-n2cccc(C)c2=O)cn1]

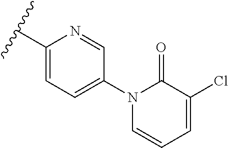 [SMILES: *c1ccc(-n2cccc(Cl)c2=O)cn1]

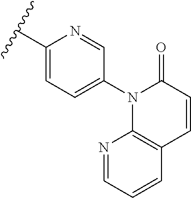 [SMILES: *c1ccc(-n2c(=O)ccc3cccnc32)cn1]

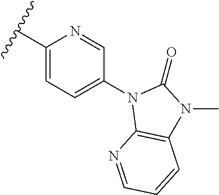 [SMILES: *c1ccc(-n2c(=O)n(C)c3cccnc32)cn1]

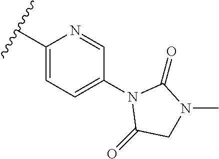 [SMILES: *c1ccc(N2C(=O)CN(C)C2=O)cn1]

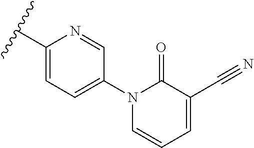 [SMILES: *c1ccc(-n2cccc(C#N)c2=O)cn1]

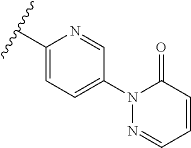 [SMILES: *c1ccc(-n2ncccc2=O)cn1]

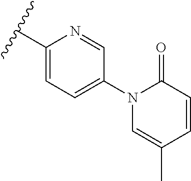 [SMILES: *c1ccc(-n2cc(C)ccc2=O)cn1]

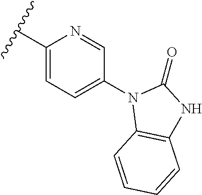 [SMILES: *c1ccc(-n2c(=O)[nH]c3ccccc32)cn1]

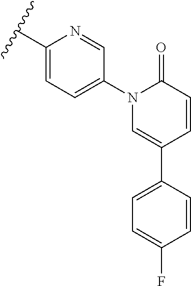 [SMILES: *c1ccc(-n2cc(-c3ccc(F)cc3)ccc2=O)cn1]

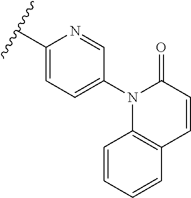 [SMILES: *c1ccc(-n2c(=O)ccc3ccccc32)cn1]

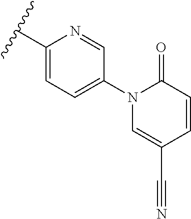 [SMILES: *c1ccc(-n2cc(C#N)ccc2=O)cn1]

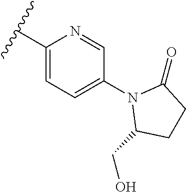

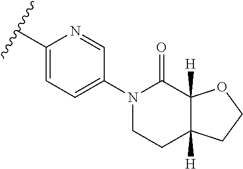 [SMILES: *c1ccc(N2CC[C@H]3CCO[C@H]3C2=O)cn1]

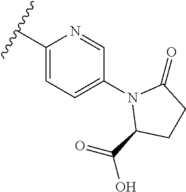 [SMILES: *c1ccc(N2C(=O)CC[C@H]2C(=O)O)cn1]

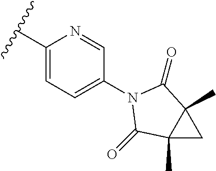 [SMILES: *c1ccc(N2C(=O)[C@]3(C)C[C@]3(C)C2=O)cn1]

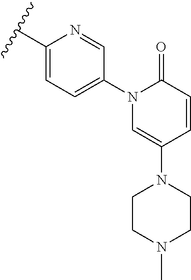 [SMILES: *c1ccc(-n2cc(N3CCN(C)CC3)ccc2=O)cn1]

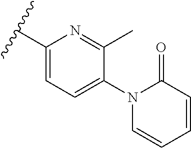 [SMILES: *c1ccc(-n2ccccc2=O)c(C)n1]

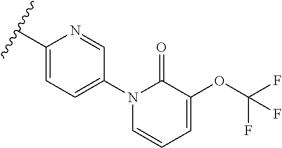 [SMILES: *c1ccc(-n2cccc(OC(F)(F)F)c2=O)cn1]

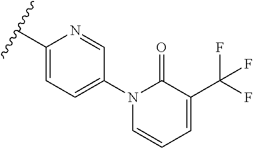 [SMILES: *c1ccc(-n2cccc(C(F)(F)F)c2=O)cn1]

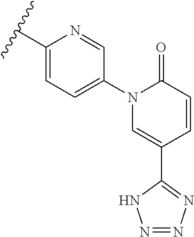 [SMILES: *c1ccc(-n2cc(-c3nnn[nH]3)ccc2=O)cn1]

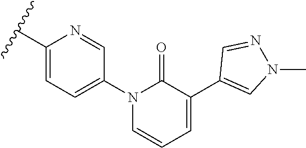 [SMILES: *c1ccc(-n2cccc(-c3cnn(C)c3)c2=O)cn1]

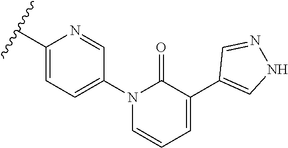 [SMILES: *c1ccc(-n2cccc(-c3cn[nH]c3)c2=O)cn1]

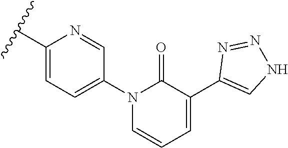 [SMILES: *c1ccc(-n2cccc(-c3c[nH]nn3)c2=O)cn1]

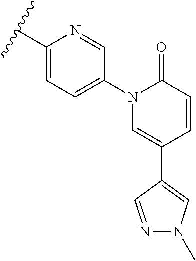 [SMILES: *c1ccc(-n2cc(-c3cnn(C)c3)ccc2=O)cn1]

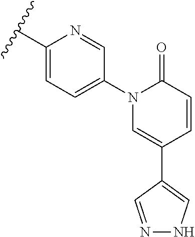 [SMILES: *c1ccc(-n2cc(-c3cn[nH]c3)ccc2=O)cn1]

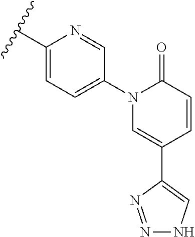 [SMILES: *c1ccc(-n2cc(-c3c[nH]nn3)ccc2=O)cn1]

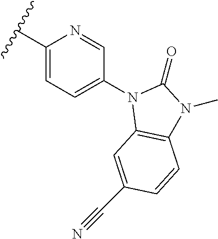 [SMILES: *c1ccc(-n2c(=O)n(C)c3ccc(C#N)cc32)cn1]

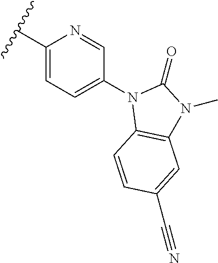 [SMILES: *c1ccc(-n2c(=O)n(C)c3cc(C#N)ccc32)cn1]

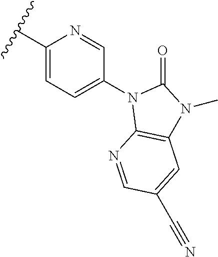 [SMILES: *c1ccc(-n2c(=O)n(C)c3cc(C#N)cnc32)cn1]

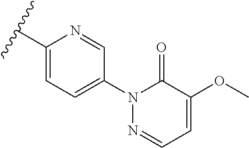 [SMILES: *c1ccc(-n2nccc(OC)c2=O)cn1]

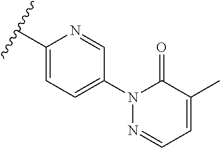 [SMILES: *c1ccc(-n2nccc(C)c2=O)cn1]

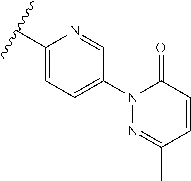 [SMILES: *c1ccc(-n2nc(C)ccc2=O)cn1]

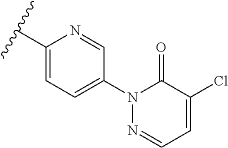 [SMILES: *c1ccc(-n2nccc(Cl)c2=O)cn1]

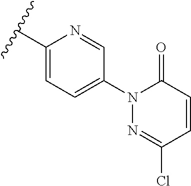 [SMILES: *c1ccc(-n2nc(Cl)ccc2=O)cn1]

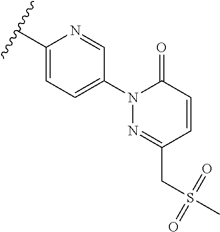 [SMILES: *c1ccc(-n2nc(CS(C)(=O)=O)ccc2=O)cn1]

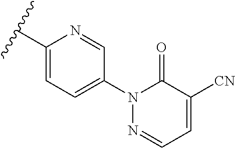 [SMILES: *c1ccc(-n2nccc(C#N)c2=O)cn1]

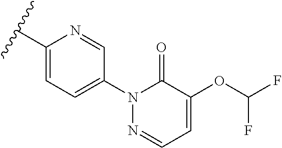 [SMILES: *c1ccc(-n2nccc(OC(F)F)c2=O)cn1]

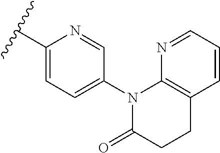 [SMILES: *c1ccc(N2C(=O)CCc3cccnc32)cn1]

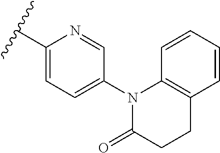 [SMILES: *c1ccc(N2C(=O)CCc3ccccc32)cn1]

In other embodiments the compound of formula A-B—C is of the Formula (I-A):

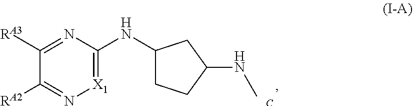

wherein X¹ is C—R^{A1}.

In some embodiments R^{A1} is H, OH, CN, Br, Cl, optionally substituted —OMe, —O-ethyl, methyl or ethyl, where the optional substituents on the —OMe, O-ethyl, methyl or ethyl groups are selected from OH, CN, or one or more halo groups. In some embodiments R^{A1} is H, CN, OH, Br, Cl, optionally substituted —OMe, —O-ethyl, methyl or ethyl, where the optional substituents on the —OMe, O-ethyl, methyl or ethyl groups are selected from OH, CN, or one or more halo groups. In further embodiments R^{A1} is optionally substituted O-methyl wherein the optional substituents are one or more F groups. In further embodiments R^{A1} is H. In further embodiments R^{A1} is OH.

In some embodiments R^{A2} is selected from the group consisting of: Br, Cl, CN, H, —C(═O)CH₃, C₁₋₆ alkyl amido which alkyl amido is optionally substituted by C₁₋₃alkyl amido, a thioalkyl optionally substituted by C₁₋₃ alkyl ester, CN, C₂₋₃ alkynyl, C₄₋₆ heterocyclyl, C₁ 3 alkyl which alkyl is optionally substituted with one or more halo or OH groups, optionally substituted methoxy, methyl, ethyl or cyclopropyl wherein the optional substituents are selected from OH, CN, or one or more halo groups, an optionally substituted methoxy or ethoxy wherein the optional substituents are selected from alkyl amido or one or more halo groups. In some embodiments R^{A2} is selected from the group consisting of: Br, Cl, CN, H, —C(═O)CH₃, C₁₋₆ alkyl amido which alkyl amido is optionally substituted by C₁₋₃alkyl amido, CN, C₂₋₃ alkynyl, C₄₋₆ heterocyclyl, C₁ 3 alkyl which alkyl is optionally substituted with one or more halo or OH groups, optionally substituted methoxy, methyl, ethyl or cyclopropyl wherein the optional substituents are selected from OH, CN, or one or more halo groups, an optionally substituted methoxy or ethoxy wherein the optional substituents are selected from alkyl amido or one or more halo groups. In further embodiments R^{A2} is selected from CN, methyl, C₁, —C(═O)CH₃, —C(═O)OCH₂CH₃, cyclopropyl, —C(═O)NHCH₂C(═O)NH₂, —C(═O)NHCH₂CHCH, —C(═O)NH-oxetane, —C(═O)NHCH₂CHF₂, —C(═O)NHCH₂CH₂OH, —C(═O)NHCH₂CH₃, —C(═O)NH₂, —C(═O)NHCH₃, —C(═O)N(CH₃)₂, —OCF₂H, H, —OMe, —OCF₃. In some embodiments R^{A2} is selected from the group consisting of CN, methyl, C₁, —C(═O)CH₃, OCHF₂, cyclopropyl, OCF₃, OCH₃, H, —C(═O)NH(CH₃), S—CH₃, —S—CH₂CH₃ or —S—CH₂—C(═O)—O—CH₃.

In some embodiments R^{A3} is selected from the group consisting of CN, Br, Cl, OH, H, CF₃, C₁₋₂ alkyl, C₁₋₂alkoxy, NH₂. In further embodiments R^{A3} is selected from H, methyl and CN.

In other embodiments R^{A3} and R^{A2} together with the carbon atoms to which they are bound form an optionally substituted C carboaromatic ring or C₅₋₇ heteroaromatic ring, the optional substituents are selected from NH₂, C₁₋₆ alkyl, C₁₋₆ alkoxy and halo. In other embodiments the optional substituents are selected from NH₂, methyl, ethyl, OMe, F, Cl and Br.

In some embodiments R^{A3} and R^{A2} together with the carbon atoms to which they are bound form an optionally substituted pyridine, an optionally substituted benzene, a pyrrole or a pyrazole. In some embodiments R^{A2} and R^{A3} together form an unsubstituted 2-pyrazole, a 2-pyrrole substituted by methyl, pyridine optionally substituted by NH₂ or benzene optionally substituted by Cl, F or OMe.

In some embodiments C is an optionally substituted pyridinyl, pyrazinyl or pyrimidinyl. In some embodiments the optional substituents are selected from:
(i) C₆₋₁₀ carboaryl, C₄₋₁₀ carbocyclyl, C₅₋₁₀ heteroaryl, C₅₋₁₀ heterocyclyl, or C₅₋₁₀ bridged heterocycle, spiro C₆₋₁₂ heterocyclyl or a spiro C₆₋₁₂ carbocyclyl, which are themselves optionally substituted by one or more of the groups selected from:
a) one or two ═O groups;
b) one or more halo groups;
c) CN, NH₂, OH;
d) one or more C₁₋₆ alkyl groups including branched and cyclic and with an optional substituent selected from OH, C₁₋₆ alkyl sulfonyl or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl with an optional methyl substituent;
i) C₄₋₁₀ carbocyclyl with an optional methyl or ═O substituent;
j) C₆₋₁₀carboaryl with optional substituents of one or more halo groups;
k) carboxy, CH₂-carboxy;
l) P(═O)Me₂;
(ii) one or more of the groups selected from carboxy, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido, di-C₁₋₆ alkyl sulfonamido.

In some embodiments C is an optionally substituted pyridinyl, pyrazinyl or pyrimidinyl. In some embodiments the optional substituents are selected from:
(i) C₆₋₁₀ carboaryl, C₄₋₁₀carbocyclyl, C₅₋₁₀ heteroaryl, C₅₋₁₀ heterocyclyl, or C₅₋₁₀ bridged heterocycle, spiro C₆₋₁₂ heterocyclyl or a spiro C₆₋₁₂ carbocyclyl, which are themselves optionally substituted by one or more of the groups selected from:
a) one or two ═O groups;
b) one or more halo groups;
c) CN, NH₂, OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl;
i) C₄₋₁₀ carbocyclyl with an optional methyl or ═O substituent;
j) C₆₋₁₀carboaryl with optional substituents of one or more halo groups;
k) carboxy, CH₂-carboxy;
l) P(═O)Me₂;
(ii) one or more of the groups selected from carboxy, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido, di-C₁₋₆ alkyl sulfonamido.

In other embodiments the compound of formula A-B—C is of the Formula (I-B):

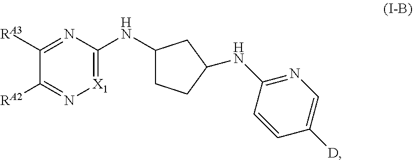

wherein X¹, R^{A2} and R^{A3} are as defined (I-A).

In some embodiments Formula (I-B) can be Formula (I-Ba) or (I-Bb) as shown below:

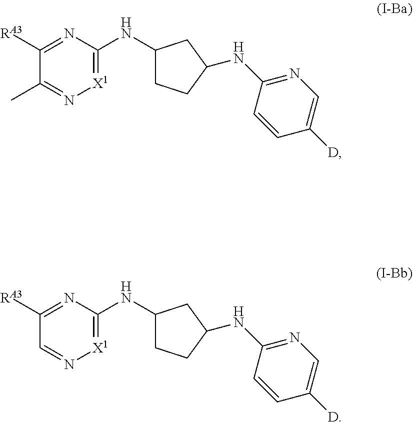

In some embodiments D is C₆₋₁₀ carboaryl, C₄₋₁₀ carbocyclyl, C₅₋₁₀ heteroaryl or C₅₋₁₀ heterocyclyl which are themselves optionally substituted by one or more groups selected from the following:
a) one or two ═O groups;
b) one or more halo groups;
c) CN, NH₂, OH;
d) one or more C₁₋₆ alkyl groups including branched and cyclic and with an optional substituent selected from OH, C₁₋₆ alkyl sulfonyl or one or more halo groups;
e) C₁₋₆ alkoxy with optional substituents of one or more halo groups;
f) C₁₋₆alkylester;
g) C₅₋₆ heterocyclyl with an optional methyl, OH or ═O substituent;
h) C₅₋₆ heteroaryl optionally substituted by methyl;
i) C₄₋₁₀ carbocyclyl with an optional methyl or ═O substituent;
j) C₆₋₁₀carboaryl with optional substituents of one or more halo groups;
l) carboxy, CH₂-carboxy
m) P(═O)Me₂.

In some embodiments D is C₆₋₁₀carboaryl, C₄₋₁₀carbocyclyl, C₅₋₁₀ heteroaryl or C₅₋₁₀ heterocyclyl which are themselves optionally substituted by one or more groups selected from the following:
a) one or two ═O groups;
b) one or more halo groups;
c) CN, NH₂, OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl;
i) C₄₋₁₀ carbocyclyl with an optional methyl or ═O substituent;
j) C₆₋₁₀carboaryl with optional substituents of one or more halo groups;
l) carboxy, CH₂-carboxy
m) P(═O)Me₂.

In some embodiments D is an optionally substituted pyridine-2-one. In some embodiments the optional substituents are selected from OMe, Cl, F, pyrazole, triazole, tetrazole, methyl, trifluoromethyl (CF₃), OCF₃, carboxy (C(═O)OH), CN, phenyl with an optional F substituent at the para position or a piperazine with a methyl substituent. In some embodiments the pyridine-2-one is unsubstituted. In some embodiments the optional substituents are selected from OMe, carboxy, Cl, methyl optionally substituted with —S(═O)₂CH₃, CN, phenyl substituted by F, piperazinyl substituted by methyl, CF₃, OCF₃, pyrazolyl optionally substituted by methyl, tetrazolyl or triazolyl. In some embodiments the pyridine-2-one is pyridin-2 (1H-one.

In some embodiments D is an optionally substituted 6 membered heteroaryl which contains one or two N atoms, one of which is bonded to C, which is substituted by ═O at the ortho position and the other optional substituents are selected from methyl, OMe, piperazinyl substituted by methyl, C(═O)OH (carboxy), Cl, F, pyrazolyl, triazolyl, tetrazolyl, phenyl substituted by fluoro, CN, CF₃ or O—CF₃. In some embodiments the other optional substituents are selected from methyl optionally substituted with —S(═O)₂CH₃, OMe, piperazinyl substituted by methyl, C(═O)OH (carboxy), Cl, phenyl substituted by fluoro, CN, CF₃, O—CF₃, pyrazole optionally substituted by methyl, triazole or tetrazole.

In other embodiments the compound of formula A-B—C is of the Formula (I-C):

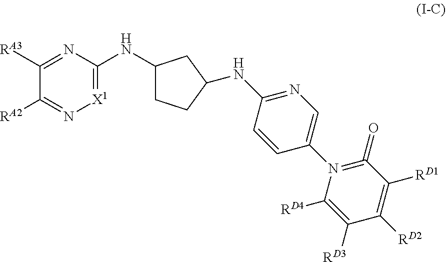

wherein X¹, R^{A2} and R^{A3} are as defined (I-A).

In some embodiments one or two of R^{D1}, R^{D2}, R^{D3} and R^{D4} are selected from a C₁₋₆ alkyl optionally substituted by one or more halo groups; C₁₋₆ alkoxy optionally substituted by one or more halo groups, C₅₋₆ heterocyclyl or C₅₋₆heteroaryl with an optional methyl substituent, carboxy, ═O, halo, NH₂, CN, or phenyl optionally substituted by one or more halo atoms or C₁₋₆ alkyl optionally substituted by C₁₋₆ alkyl sulfonyl; or

wherein R^{D3} and R^{D4} form an optionally substituted 6 membered carboaromatic, heterocycle or heteroaromatic ring wherein the optional substituents are selected from OH, methyl, OMe, halo, CN, P(═O)Me₂ and C(═O)OH;

or wherein R^{D1}, R^{D2}, R^{D3} and R^{D4} are all H.

In some embodiments one or two of R^{D1}, R^{D2}, R^{D3} and R^{D4} are selected from a C₁₋₆ alkyl optionally substituted by one or more halo groups or —S(═O)₂CH₃; C₁₋₆ alkoxy optionally substituted by one or more halo groups, C₅₋₆ heterocyclyl or C₅₋₆ heteroaryl with an optional methyl substituent, carboxy, ═O, halo, NH₂, CN, or phenyl optionally substituted by one or more halo atoms; or

wherein R^{D3} and R^{D4} form an optionally substituted 6 membered carboaromatic, heterocycle or heteroaromatic ring wherein the optional substituents are selected from OH, methyl, OMe, halo, C(═O)OH.

In another embodiment one or two of R^{D1}, R^{D2}, R^{D3} and R^{D4} are selected from methyl, OMe, halo, C(═O)OH, CN, CF₃, OCF₃, OCHF₂, pyrazolyl, triazolyl, tetrazolyl and the rest are H. In another embodiment one or two of R^{D1}, R^{D2}, R^{D3} and R^{D4} are selected from methyl optionally substituted with —S(═O)₂CH₃, OMe, halo, C(═O)OH, CN, CF₃, OCF₃, pyrazolyl optionally substituted by methyl, triazolyl, tetrazolyl, phenyl substituted by fluoro, piperazine substituted by methyl and the rest of R^{D1}, R^{D2}, R^{D3} and R^{D4} are H.

In one embodiment all of R^{D1}, R^{D2}, R^{D3} and R^{D4} are H.

In some embodiments R^{D3} and R^{D4} form an unsubstituted benzene ring or an unsubstituted pyridine ring.

In other embodiments the compound of formula A-B—C is of Formula (I-D):

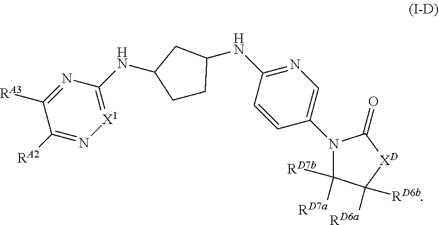

In some embodiments X¹, R^{A2} and R^{A3} are as defined (I-A).

In some embodiments X^{D} is NR^{D5a} or CR^{D5a}R^{D5b};

R^{D5a} is selected from H or methyl;

either R^{D5b} and R^{D6b} are both H or together they are —CH₂—;

R^{D6a} is selected from H, ═O, methyl, CH₂OH or C(═O)OH;

R^{D7a} is selected from H, ═O, methyl, CH₂OH or C(═O)OH;

R^{D7b} is H, wherein when R^{D7a} is ═O, R^{D7b} is absent;

or wherein R^{D6a} and R^{D7a} together form a benzene ring or a C heteroaromatic ring which is

optionally substituted by CN, P(═O)Me₂ or carboxy and R^{D6b} and R^{D7b} are absent.

In some embodiments the compound is of Formula (I)

A-B—C (I)

or a pharmaceutically acceptable salt, tautomeric forms or stereoisomers thereof,

wherein A is of the following:

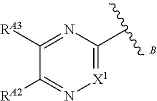

wherein the wavy line indicates the point of attachment to B;

X¹ is C—R^{A1};

R^{A1} is selected from the group consisting of:
(i) H;
(ii) halo;
(iii) CN;
(iv) C₁₋₆ hydrocarbon, optionally substituted by one or more OH, CN, C₁₋₆ acyl, C₁₋₆alkoxy or one or more halo groups;
(v) C₁₋₆ alkoxy, optionally substituted by OH, one or more halo groups, C₁₋₆ alkyl amido;
(vi) C₁₋₆ alkylester;
(vii) C₁₋₆ alkyl acyl; and
(viii) OH;

R^{A2} is selected from the group consisting of:
(i) H;
(ii) halo;
(iii) CN;
(iv) C₁₋₆ hydrocarbon, optionally substituted by OH, CN, C₁₋₆acyl, C₁₋₆alkoxy or one or more halo groups;
(v) C₁₋₆ alkoxy, optionally substituted by OH, alkyl amido, or one or more halo groups;
(vi) C₁₋₆ acyl amido (wherein the acyl is optionally substituted by H or methyl);
(vii) C₁₋₆ thioalkyl;
(viii) C₁₋₆ alkyl ester;
(ix) C₁₋₆ alkyl acyl;
(x) C₄₋₅ heterocyclyl;
(xi) C₅ heteroaryl;
(xii) C₁₋₆ alkyl amido optionally substituted by C₁₋₃ alkyl amido, CN, C₂₋₃ alkynyl, C₄₋₆ heterocyclyl or C₁₋₃ alkyl wherein the C₁₋₃ alkyl is optionally substituted with one or more halo or OH groups; and
(xiii) OH;

R^{A3} is selected from the group consisting of:
(i) H;
(ii) halo;
(iii) CN;
(iv) C₁₋₆hydrocarbon optionally substituted by OH, CN, C₁₋₆ thioalkyl, C₁₋₆ alkoxy, C₁₋₆ alkyl acyl, C₁₋₆ acyloxy, carboxy, C₁₋₆ alkyl ester, C₁₋₆ alkylamino, —C(═O)NH₂, C₁₋₆ alkyl amido, C₁₋₆ alkyl acylamido, C₁₋₆ alkyl sulfinyl, C₁₋₆ alkyl sulfonyl or one or more halo groups;
(v) OH;
(vi) C₁₋₆ alkoxy, optionally substituted by OH, NH₂, C₄ heterocyclyl or one or more halo groups;
(vii) C₁₋₆ acyloxy;
(viii) C₄ heterocycyl;
(ix) —NH₂;
(x) C₁₋₆ alkylamino, optionally substituted by CN, OH or C₄ heterocyclyl;
(xi) C₁₋₆ dialkylamino, optionally substituted by —NH₂;
(xii) C₁₋₆ acylamido (where acyl substituent is H or Me);
(xiii) carbaimidoyl or methyl-carbaimidoyl;
(xiv) carboxyamino;
(xv) C₁₋₆ thioalkyl, optionally substituted by OH or NH₂;
(xvi) C₁₋₆ alkyl sulfinyl;
(xvi) C₁₋₆ alkyl sulfonyl, optionally substituted by one or more halo groups;
(xvii) C₁₋₆ sulfonimodyl;
(xviii) C₁₋₆ alkyl phosphinyl;
(xix) carboxy;
(xx) C(═O)NH₂
(xxi) C₁₋₆ alkyl ester;
(xxii) C₁₋₆ alkyl acyl, optionally substituted by one or more halo groups; and
(xxiii) C₁₋₆ alkyl amido;

or wherein R^{A3} and R^{A2} together with the carbon atoms to which they are bound form:
(i) an optionally substituted C₅₋₇ heterocycle ring;
(ii) an optionally substituted C₅₋₇ heteroaromatic ring;
(iii) an optionally substituted C₆ carboaromatic ring;
(iv) an optionally substituted C₅₋₇ carbocyclic ring
wherein the optional substituents are selected from C₁₋₆alkyl, halo, C₁₋₆ alkoxy, NH₂, C₁₋₆alkylamino, OH, and CN;

wherein B is of formula (B-1) or (B-2)
i)

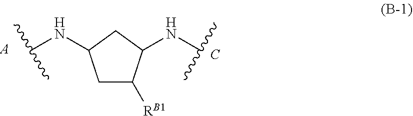

wherein the wavy line indicates the point of attachment to A and C;

R^{B1} is H, OH, ═CHCH₂—OH, —O—C₁₋₄alkyl or C₁₋₄alkyl, wherein the C₁₋₄alkyl is optionally substituted by OH or OMe;
(ii)

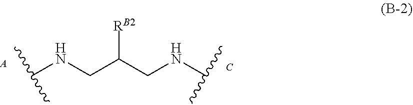

wherein the wavy line indicates the point of attachment to A and C;

R^{B2} is C₁₋₂ alkyl-OH, CH₂CONHMe or C₁₋₃ alkyl;

wherein C is selected from the group consisting of C₆₋₁₀ carboaryl, C₅₋₆ heteroaryl and C₅₋₁₀ heterocyclyl, which groups are optionally substituted by:
(i) C₆₋₁₀ carboaryl, C₄₋₁₀ carbocyclyl, C₅₋₁₀ heteroaryl C₄₋₁₀ heterocyclyl, or C₅₋₁₀ bridged heterocycle, spiro C₆₋₁₂heterocyclyl or a spiro C₆₋₁₂carbocyclyl, which are themselves optionally substituted by one or more of the following groups:
a) one or two ═O groups;
b) one or more halo groups;
c) CN, NH₂, OH;
d) one or more C₁₋₆ alkyl groups including branched and cyclic and with an optional substituent selected from OH or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl;
i) C₄₋₁₀ carbocyclyl with an optional methyl or ═O substituent;
j) C₆₋₁₀ carboaryl with optional substituents of one or more halo groups;
l) P(═O)Me₂;
m) carboxy or CH₂-carboxy; and/or
n) tetrazolyl, CH₂-tetrazolyl, 5-oxo-4H-1,2,4-oxadiazol-3-yl;
(ii) one or more groups selected from carboxy, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido, and di-C₁₋₆ alkyl sulfonamido.

In some embodiments the compound is of Formula (I)

A-B—C (I)

or a pharmaceutically acceptable salt, tautomeric forms or stereoisomers thereof,

wherein A is of the following:

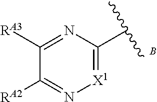

wherein the wavy line indicates the point of attachment to B;

R^{A2} is C₁₋₆ thioalkyl substituted by C₁₋₆ alkyl ester or alkyl amido substituted by OH;

C is selected from the group consisting of C₆₋₁₀ carboaryl, C₅₋₆ heteroaryl and C₅₋₁₀ heterocyclyl, which groups are optionally substituted by:
(i) a group selected from C₆₋₁₀carboaryl, C₄₋₁₀carbocyclyl, C₅₋₁₀ heteroaryl C₄₋₁₀ heterocyclyl, or C₅₋₁₀ bridged heterocycle, spiro C₆₋₁₂heterocyclyl or a spiro C₆₋₁₂ carbocyclyl, which are themselves optionally substituted by one or more of the following groups:

one or more C₁₋₆ alkyl groups including branched and cyclic with a C₁₋₆ alkyl sulfonyl substituent or a C₅₋₆ heteroaryl substituted with methyl;

wherein X¹, R^{A3} and B are as defined above.

In some embodiments the compound of Formula (I) is selected from the following in Table 1.

TABLE 1

| Ex | Structure | Name |
|---|---|---|
| 1 | 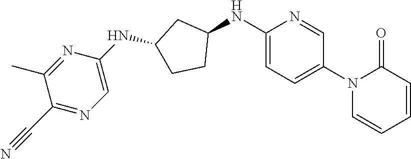 [SMILES: Cc1nc(N[C@H]2CC[C@H](Nc3ccc(-n4ccccc4=O)cn3)C2)cnc1C#N] | 3-Methyl-5-(((1S,3S)-3-((2-oxo-2H- [1,3′-bipyridin]-6′- yl)amino)cyclopentyl)amino)pyrazine- 2-carbonitrile |
| 2 | 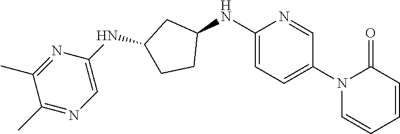 [SMILES: Cc1ncc(N[C@H]2CC[C@H](Nc3ccc(-n4ccccc4=O)cn3)C2)nc1C] | 6′-(((1S,3S)-3-((5,6-Dimethylpyrazin- 2-yl)amino)cyclopentyl)amino)-2H- [1,3′-bipyridin]-2-one |
| 3 | 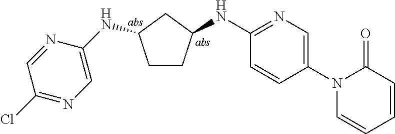 [SMILES: O=c1ccccn1-c1ccc(N[C@H]2CC[C@H](Nc3cnc(Cl)cn3)C2)nc1] | 6′-(((1S,3S)-3-((5-Chloropyrazin-2- yl)amino)cyclopentyl)amino)-2H- [1,3′-bipyridin]-2-one |
| 4 | 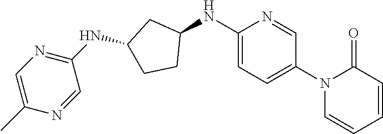 [SMILES: Cc1cnc(N[C@H]2CC[C@H](Nc3ccc(-n4ccccc4=O)cn3)C2)cn1] | 6′-(((1S,3S)-3-((5-Methylpyrazin-2- yl)amino)cyclopentyl)amino)-2H- [1,3′-bipyridin]-2-one |
| 5 | 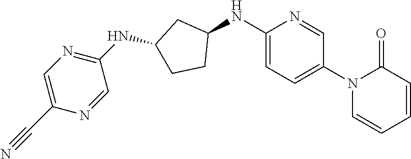 [SMILES: N#Cc1cnc(N[C@H]2CC[C@H](Nc3ccc(-n4ccccc4=O)cn3)C2)cn1] | 5-(((1S,3S)-3-((2-Oxo-2H-[1,3′- bipyridin]-6′- yl)amino)cyclopentyl)amino)pyrazine- 2-carbonitrile |
| 6 | 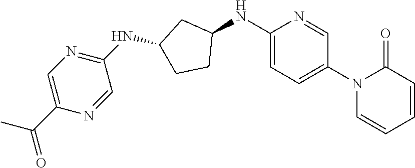 [SMILES: CC(=O)c1cnc(N[C@H]2CC[C@H](Nc3ccc(-n4ccccc4=O)cn3)C2)cn1] | 6′-(((1S,3S)-3-((5-Acetylpyrazin-2- yl)amino)cyclopentyl)amino)-2H- [1,3′-bipyridin]-2-one |
| 7 | 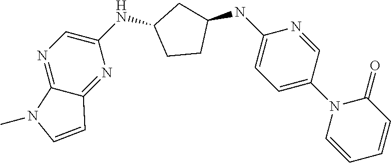 [SMILES: Cn1ccc2nc(N[C@H]3CC[C@H](Nc4ccc(-n5ccccc5=O)cn4)C3)cnc21] | 6′-(((1S,3S)-3-((5-Methyl-5H- pyrrolo[2,3-b]pyrazin-2- yl)amino)cyclopentyl)amino)-2H- [1,3′-bipyridin]-2-one |
| 8 | 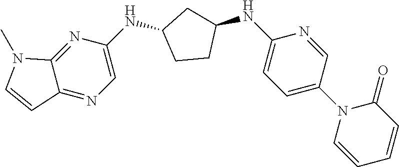 [SMILES: Cn1ccc2ncc(N[C@H]3CC[C@H](Nc4ccc(-n5ccccc5=O)cn4)C3)nc21] | 6′-(((1S,3S)-3-((5-Methyl-5H- pyrrolo[2,3-b]pyrazin-3- yl)amino)cyclopentyl)amino)-2H- [1,3′-bipyridin]-2-one |
| 9 | 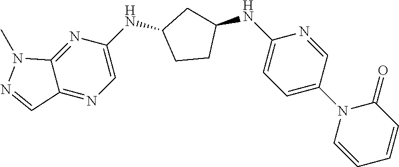 [SMILES: Cn1ncc2ncc(N[C@H]3CC[C@H](Nc4ccc(-n5ccccc5=O)cn4)C3)nc21] | 6′-(((1S,3S)-3-((1-Methyl-1H- pyrazolo[3,4-b]pyrazin-6- yl)amino)cyclopentyl)amino)-2H- [1,3′-bipyridin]-2-one |
| 11 | 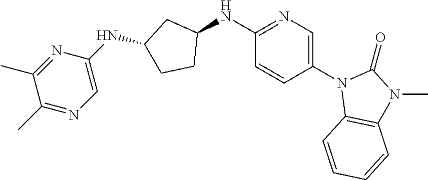 | 1-(6-(((1S,3S)-3-((5,6- Dimethylpyrazin-2- yl)amino)cyclopentyl)amino)pyridin- 3-yl)-3-methyl-1,3-dihydro-2H- benzo[d]imidazol-2-one |
| 12 | 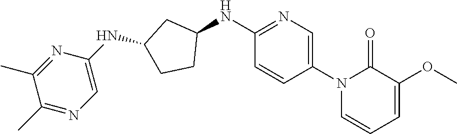 | 6′-(((1S,3S)-3-((5,6-Dimethylpyrazin- 2-yl)amino)cyclopentyl)amino)-3- methoxy-2H-[1,3′-bipyridin]-2-one |
| 13 | 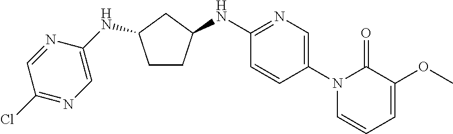 [SMILES: COc1cccn(-c2ccc(N[C@H]3CC[C@H](Nc4cnc(Cl)cn4)C3)nc2)c1=O] | 6′-(((1S,3S)-3-((5-Chloropyrazin-2- yl)amino)cyclopentyl)amino)-3- methoxy-2H-[1,3′-bipyridin]-2-one |
| 14 | 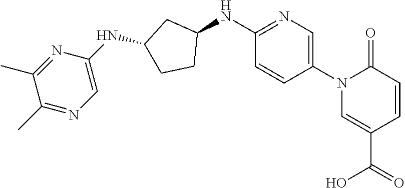 [SMILES: Cc1ncc(N[C@H]2CC[C@H](Nc3ccc(-n4cc(C(=O)O)ccc4=O)cn3)C2)nc1C] | 6′-(((1S,3S)-3-((5,6-Dimethylpyrazin- 2-yl)amino)cyclopentyl)amino)-2-oxo- 2H-[1,3′-bipyridine]-5-carboxylic acid |
| 15 | 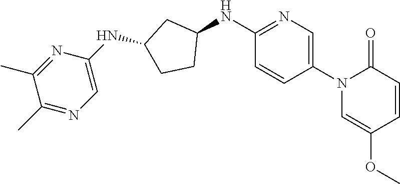 | 6′-(((1S,3S)-3-((5,6-Dimethylpyrazin- 2-yl)amino)cyclopentyl)amino)-5- methoxy-2H-[1,3′-bipyridin]-2-one |
| 16 | 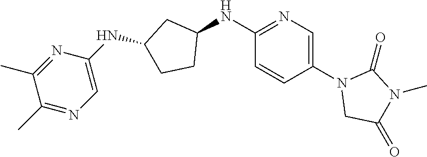 | 1-(6-(((1S,3S)-3-((5,6- Dimethylpyrazin-2- yl)amino)cyclopentyl)amino)pyridin- 3-yl)-3-methylimidazolidine-2,4-dione |
| 17 | 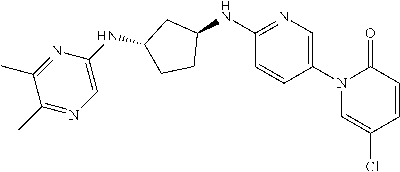 [SMILES: Cc1ncc(N[C@H]2CC[C@H](Nc3ccc(-n4cc(Cl)ccc4=O)cn3)C2)nc1C] | 5-Chloro-6′-(((1S,3S)-3-((5,6- dimethylpyrazin-2- yl)amino)cyclopentyl)amino)-2H- [1,3′-bipyridin]-2-one |
| 18 | 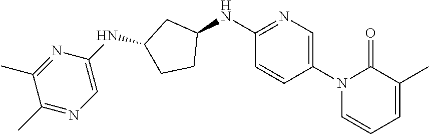 | 6′-(((1S,3S)-3-((5,6-Dimethylpyrazin- 2-yl)amino)cyclopentyl)amino)-3- methyl-2H-[1,3′-bipyridin]-2-one |
| 19 | 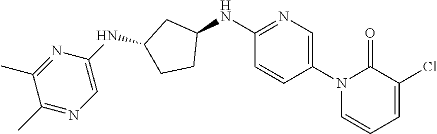 [SMILES: Cc1ncc(N[C@H]2CC[C@H](Nc3ccc(-n4cccc(Cl)c4=O)cn3)C2)nc1C] | 3-Chloro-6′-(((1S,3S)-3-((5,6- dimethylpyrazin-2- yl)amino)cyclopentyl)amino)-2H- [1,3′-bipyridin]-2-one |
| 20 | 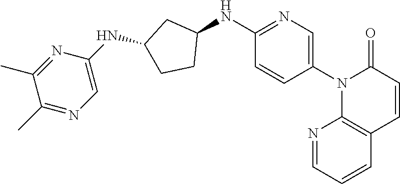 [SMILES: Cc1ncc(N[C@H]2CC[C@H](Nc3ccc(-n4c(=O)ccc5cccnc54)cn3)C2)nc1C] | 1-(6-(((1S,3S)-3-((5,6- Dimethylpyrazin-2- yl)amino)cyclopentyl)amino)pyridin- 3-yl)-1,8-naphthyridin-2(1H)-one |
| 21 | 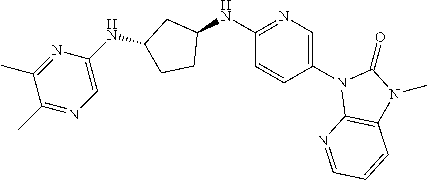 | 3-(6-(((1S,3S)-3-((5,6- Dimethylpyrazin-2- yl)amino)cyclopentyl)amino)pyridin- 3-yl)-1-methyl-1,3-dihydro-2H- imidazo[4,5-b]pyridin-2-one |
| 22 | 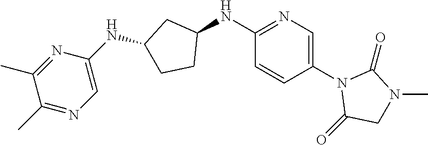 | 3-(6-(((1S,3S)-3-((5,6- Dimethylpyrazin-2- yl)amino)cyclopentyl)amino)pyridin- 3-yl)-1-methylimidazolidine-2,4-dione |
| 23 | 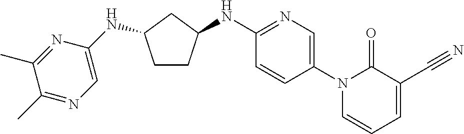 [SMILES: Cc1ncc(N[C@H]2CC[C@H](Nc3ccc(-n4cccc(C#N)c4=O)cn3)C2)nc1C] | 6′-(((1S,3S)-3-((5,6-Dimethylpyrazin- 2-yl)amino)cyclopentyl)amino)-2-oxo- 2H-[1,3′-bipyridine]-3-carbonitrile |
| 24 | 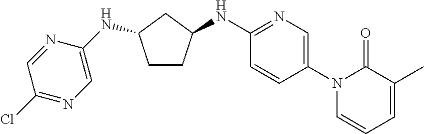 [SMILES: Cc1cccn(-c2ccc(N[C@H]3CC[C@H](Nc4cnc(Cl)cn4)C3)nc2)c1=O] | 6′-(((1S,3S)-3-((5-Chloropyrazin-2- yl)amino)cyclopentyl)amino)-3- methyl-2H-[1,3′-bipyridin]-2-one |
| 25 | 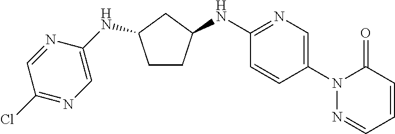 [SMILES: O=c1cccnn1-c1ccc(N[C@H]2CC[C@H](Nc3cnc(Cl)cn3)C2)nc1] | 2-(6-(((1S,3S)-3-((5-Chloropyrazin-2- yl)amino)cyclopentyl)amino)pyridin- 3-yl)pyridazin-3(2H)-one |
| 26 | 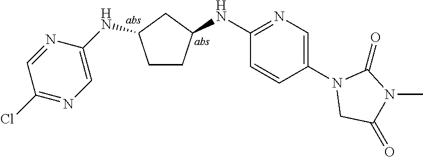 [SMILES: CN1C(=O)CN(c2ccc(N[C@H]3CC[C@H](Nc4cnc(Cl)cn4)C3)nc2)C1=O] | 1-(6-(((1S,3S)-3-((5-Chloropyrazin-2- yl)amino)cyclopentyl)amino)pyridin- 3-yl)-3-methylimidazolidine-2,4-dione |
| 27 | 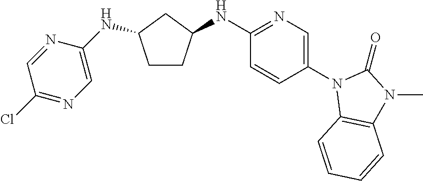 [SMILES: Cn1c(=O)n(-c2ccc(N[C@H]3CC[C@H](Nc4cnc(Cl)cn4)C3)nc2)c2ccccc21] | 1-(6-(((1S,3S)-3-((5-Chloropyrazin-2- yl)amino)cyclopentyl)amino)pyridin- 3-yl)-3-methyl-1,3-dihydro-2H- benzo[d]imidazol-2-one |
| 28 | 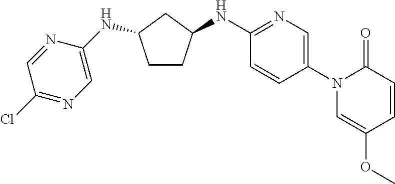 [SMILES: COc1ccc(=O)n(-c2ccc(N[C@H]3CC[C@H](Nc4cnc(Cl)cn4)C3)nc2)c1] | 6′-(((1S,3S)-3-((5-Chloropyrazin-2- yl)amino)cyclopentyl)amino)-5- methoxy-2H-[1,3′-bipyridin]-2-one |
| 29 | 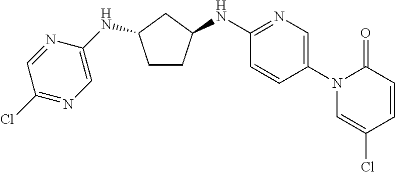 [SMILES: O=c1ccc(Cl)cn1-c1ccc(N[C@H]2CC[C@H](Nc3cnc(Cl)cn3)C2)nc1] | 5-Chloro-6′-(((1S,3S)-3-((5- chloropyrazin-2- yl)amino)cyclopentyl)amino)-2H- [1,3′-bipyridin]-2-one |
| 30 | 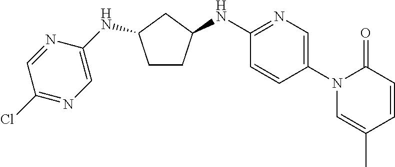 [SMILES: Cc1ccc(=O)n(-c2ccc(N[C@H]3CC[C@H](Nc4cnc(Cl)cn4)C3)nc2)c1] | 6′-(((1S,3S)-3-((5-Chloropyrazin-2- yl)amino)cyclopentyl)amino)-5- methyl-2H-[1,3′-bipyridin]-2-one |
| 31 | 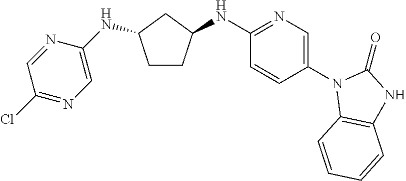 [SMILES: O=c1[nH]c2ccccc2n1-c1ccc(N[C@H]2CC[C@H](Nc3cnc(Cl)cn3)C2)nc1] | 1-(6-(((1S,3S)-3-((5-Chloropyrazin-2- yl)amino)cyclopentyl)amino)pyridin- 3-yl)-1,3-dihydro-2H- benzo[d]imidazol-2-one |
| 32 | 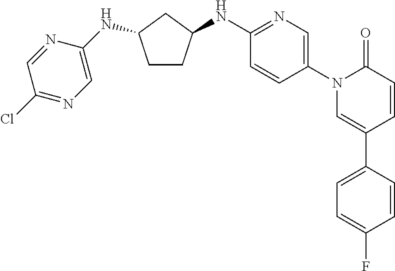 [SMILES: O=c1ccc(-c2ccc(F)cc2)cn1-c1ccc(N[C@H]2CC[C@H](Nc3cnc(Cl)cn3)C2)nc1] | 6′-(((1S,3S)-3-((5-Chloropyrazin-2- yl)amino)cyclopentyl)amino)-5-(4- fluorophenyl)-2H-[1,3′-bipyridin]-2- one |
| 33 | 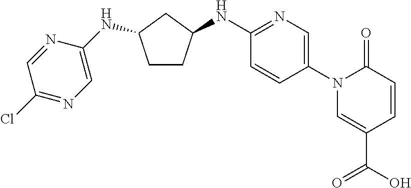 [SMILES: O=C(O)c1ccc(=O)n(-c2ccc(N[C@H]3CC[C@H](Nc4cnc(Cl)cn4)C3)nc2)c1] | 6′-(((1S,3S)-3-((5-Chloropyrazin-2- yl)amino)cyclopentyl)amino)-2-oxo- 2H-[1,3′-bipyridine]-5-carboxylic acid |
| 34 | 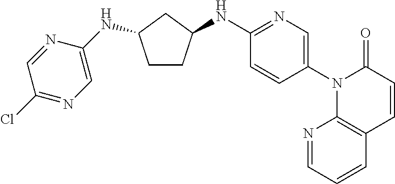 [SMILES: O=c1ccc2cccnc2n1-c1ccc(N[C@H]2CC[C@H](Nc3cnc(Cl)cn3)C2)nc1] | 1-(6-(((1S,3S)-3-((5-Chloropyrazin-2- yl)amino)cyclopentyl)amino)pyridin- 3-yl)-1,8-naphthyridin-2(1H)-one |
| 35 | 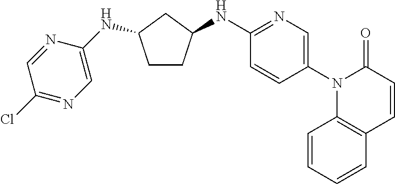 [SMILES: O=c1ccc2ccccc2n1-c1ccc(N[C@H]2CC[C@H](Nc3cnc(Cl)cn3)C2)nc1] | 1-(6-(((1S,3S)-3-((5-Chloropyrazin-2- yl)amino)cyclopentyl)amino)pyridin- 3-yl)quinolin-2(1 H)-one |
| 36 | 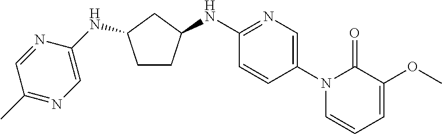 | 3-Methoxy-6′-(((1S,3S)-3-((5- methylpyrazin-2- yl)amino)cyclopentyl)amino)-2H- [1,3′-bipyridin]-2-one |
| 37 | 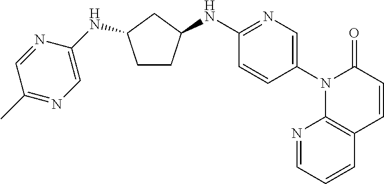 [SMILES: Cc1cnc(N[C@H]2CC[C@H](Nc3ccc(-n4c(=O)ccc5cccnc54)cn3)C2)cn1] | 1-(6-(((1S,3S)-3-((5-Methylpyrazin-2- yl)amino)cyclopentyl)amino)pyridin- 3-yl)-1,8-naphthyridin-2(1 H)-one |
| 38 | 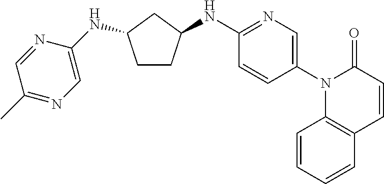 [SMILES: Cc1cnc(N[C@H]2CC[C@H](Nc3ccc(-n4c(=O)ccc5ccccc54)cn3)C2)cn1] | 1-(6-(((1S,3S)-3-((5-Methylpyrazin-2- yl)amino)cyclopentyl)amino)pyridin- 3-yl)quinolin-2(1H)-one |
| 39 | 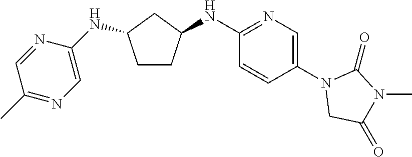 | 3-Methyl-1-(6-(((1S,3S)-3-((5- methylpyrazin-2- yl)amino)cyclopentyl)amino)pyridin- 3-yl)imidazolidine-2,4-dione |
| 40 | 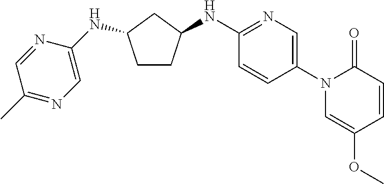 | 5-Methoxy-6′-(((1S,3S)-3-((5- methylpyrazin-2- yl)amino)cyclopentyl)amino)-2H- [1,3′-bipyridin]-2-one |
| 41 | 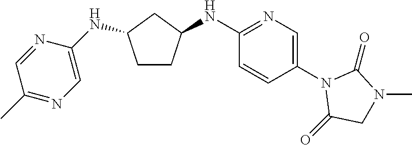 | 1-Methyl-3-(6-(((1S,3S)-3-((5- methylpyrazin-2- yl)amino)cyclopentyl)amino)pyridin- 3-yl)imidazolidine-2,4-dione |
| 42 | 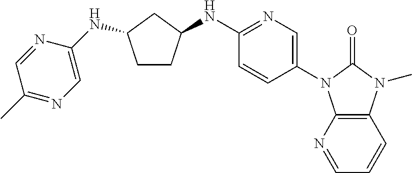 | 1-Methyl-3-(6-(((1S,3S)-3-((5- methylpyrazin-2- yl)amino)cyclopentyl)amino)pyridin- 3-yl)-1,3-dihydro-2H-imidazo[4,5- b]pyridin-2-one |
| 43 | 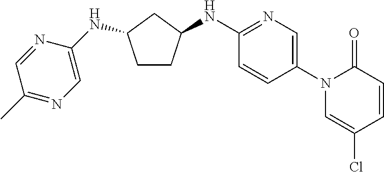 [SMILES: Cc1cnc(N[C@H]2CC[C@H](Nc3ccc(-n4cc(Cl)ccc4=O)cn3)C2)cn1] | 5-Chloro-6′-(((1S,3S)-3-((5- methylpyrazin-2- yl)amino)cyclopentyl)amino)-2H- [1,3′-bipyridin]-2-one |
| 44 | 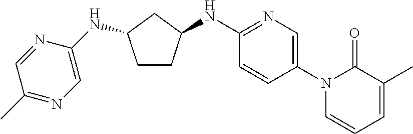 | 3-Methyl-6′-(((1S,3S)-3-((5- methylpyrazin-2- yl)amino)cyclopentyl)amino)-2H- [1,3′-bipyridin]-2-one |
| 45 | 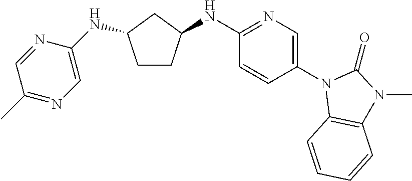 | 1-Methyl-3-(6-(((1S,3S)-3-((5- methylpyrazin-2- yl)amino)cyclopentyl)amino)pyridin- 3-yl)-1,3-dihydro-2H- benzo[d]imidazol-2-one |
| 46 | 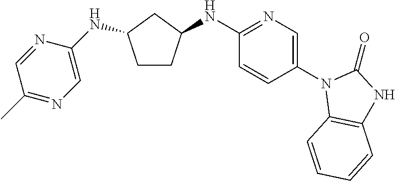 [SMILES: Cc1cnc(N[C@H]2CC[C@H](Nc3ccc(-n4c(=O)[nH]c5ccccc54)cn3)C2)cn1] | 1-(6-(((1S,3S)-3-((5-Methylpyrazin-2- yl)amino)cyclopentyl)amino)pyridin- 3-yl)-1,3-dihydro-2H- benzo[d]imidazol-2-one |
| 47 | 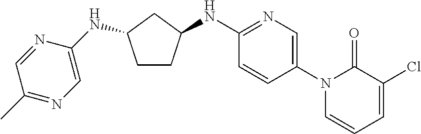 [SMILES: Cc1cnc(N[C@H]2CC[C@H](Nc3ccc(-n4cccc(Cl)c4=O)cn3)C2)cn1] | 3-Chloro-6′-(((1S,3S)-3-((5- methylpyrazin-2- yl)amino)cyclopentyl)amino)-2H- [1,3′-bipyridin]-2-one |
| 48 | 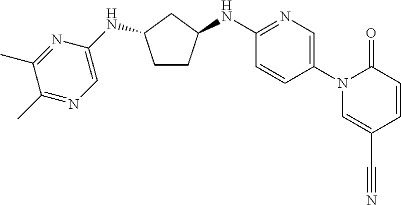 [SMILES: Cc1ncc(N[C@H]2CC[C@H](Nc3ccc(-n4cc(C#N)ccc4=O)cn3)C2)nc1C] | 6′-(((1S,3S)-3-((5,6-Dimethylpyrazin- 2-yl)amino)cyclopentyl)amino)-2-oxo- 2H-[1,3′-bipyridine]-5-carbonitrile |
| 49 | 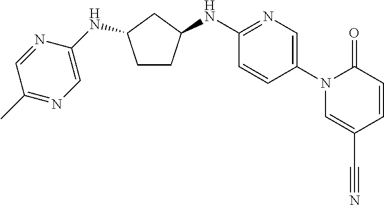 [SMILES: Cc1cnc(N[C@H]2CC[C@H](Nc3ccc(-n4cc(C#N)ccc4=O)cn3)C2)cn1] | 6′-(((1S,3S)-3-((5-Methylpyrazin-2- yl)amino)cyclopentyl)amino)-2-oxo- 2H-[1,3′-bipyridine]-5-carbonitrile |
| 77 | 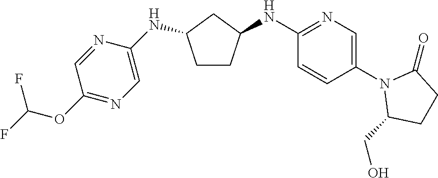 [SMILES: O=C1CC[C@H](CO)N1c1ccc(N[C@H]2CC[C@H](Nc3cnc(OC(F)F)cn3)C2)nc1] | (R)-1-(6-(((1S,3S)-3-((5- (Difluoromethoxy)pyrazin-2- yl)amino)cyclopentyl)amino)pyridin- 3-yl)-5-(hydroxymethyl)pyrrolidin-2- one |
| 78 | 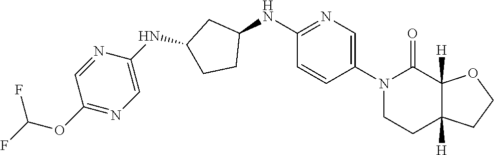 [SMILES: O=C1[C@@H]2OCC[C@@H]2CCN1c1ccc(N[C@H]2CC[C@H](Nc3cnc(OC(F)F)cn3)C2)nc1] | (3aS,7aR)-6-(6-(((1S,3S)-3-((5- (Difluoromethoxy)pyrazin-2- yl)amino)cyclopentyl)amino)pyridin- 3-yl)hexahydrofuro[2,3-c]pyridin- 7(4H)-one |
| 79 | 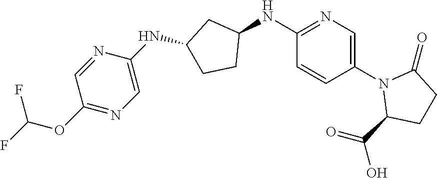 [SMILES: O=C(O)[C@@H]1CCC(=O)N1c1ccc(N[C@H]2CC[C@H](Nc3cnc(OC(F)F)cn3)C2)nc1] | (S)-1-(6-(((1S,3S)-3-((5- (Difluoromethoxy)pyrazin-2- yl)amino)cyclopentyl)amino)pyridin- 3-yl)-5-oxopyrrolidine-2-carboxylic acid |
| 80 | 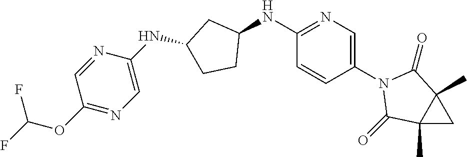 [SMILES: C[C@@]12C[C@]1(C)C(=O)N(c1ccc(N[C@H]3CC[C@H](Nc4cnc(OC(F)F)cn4)C3)nc1)C2=O] | (cis)-3-(6-(((1S,3S)-3-((5- (Difluoromethoxy)pyrazin-2- yl)amino)cyclopentyl)amino)pyridin- 3-yl)-1,5-dimethyl-3- azabicyclo[3.1.0]hexane-2,4-dione |

Further compounds are shown in Table 2:

TABLE 2

| Ex | Structure | Name |
|---|---|---|
| 81 | 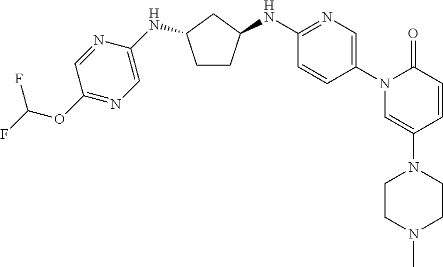 [SMILES: CN1CCN(c2ccc(=O)n(-c3ccc(N[C@H]4CC[C@H](Nc5cnc(OC(F)F)cn5)C4)nc3)c2)CC1] | 6′-(((1S,3S)-3-((5- (Difluoromethoxy)pyrazin-2- yl)amino)cyclopentyl)amino)-5-(4- methylpiperazin-1-yl)-2H-[1,3′- bipyridin]-2-one |
| 82 | 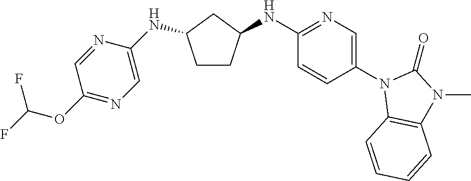 [SMILES: Cn1c(=O)n(-c2ccc(N[C@H]3CC[C@H](Nc4cnc(OC(F)F)cn4)C3)nc2)c2ccccc21] | 1-(6-(((1S,3S)-3-((5- (Difluoromethoxy)pyrazin-2- yl)amino)cyclopentyl)amino)pyridin- 3-yl)-3-methyl-1,3-dihydro-2H- benzo[d]imidazol-2-one |
| 83 | 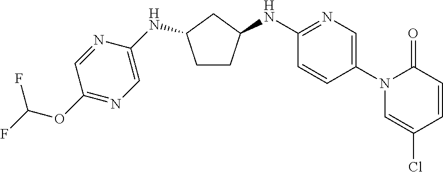 [SMILES: O=c1ccc(Cl)cn1-c1ccc(N[C@H]2CC[C@H](Nc3cnc(OC(F)F)cn3)C2)nc1] | 5-Chloro-6′-(((1S,3S)-3-((5- (difluoromethoxy)pyrazin-2- yl)amino)cyclopentyl)amino)-2H- [1,3′-bipyridin]-2-one |
| 84 | 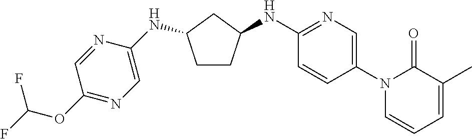 [SMILES: Cc1cccn(-c2ccc(N[C@H]3CC[C@H](Nc4cnc(OC(F)F)cn4)C3)nc2)c1=O] | 6′-(((1S,3S)-3-((5- (Difluoromethoxy)pyrazin-2- yl)amino)cyclopentyl)amino)-3- methyl-2H-[1,3′-bipyridin]-2-one |
| 85 | 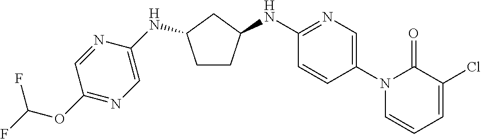 [SMILES: O=c1c(Cl)cccn1-c1ccc(N[C@H]2CC[C@H](Nc3cnc(OC(F)F)cn3)C2)nc1] | 3-Chloro-6′-(((1S,3S)-3-((5- (difluoromethoxy)pyrazin-2- yl)amino)cyclopentyl)amino)-2H- [1,3′-bipyridin]-2-one |
| 86 | 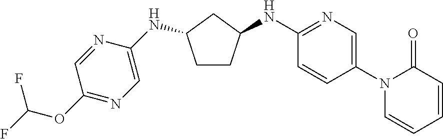 [SMILES: O=c1ccccn1-c1ccc(N[C@H]2CC[C@H](Nc3cnc(OC(F)F)cn3)C2)nc1] | 6′-(((1S,3S)-3-((5- (Difluoromethoxy)pyrazin-2- yl)amino)cyclopentyl)amino)-2H- [1,3′-bipyridin]-2-one |
| 87 | 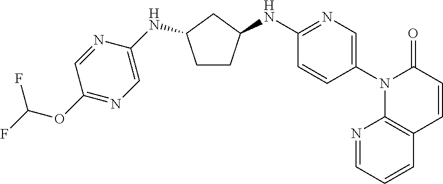 [SMILES: O=c1ccc2cccnc2n1-c1ccc(N[C@H]2CC[C@H](Nc3cnc(OC(F)F)cn3)C2)nc1] | 1-(6-(((1S,3S)-3-((5- (Difluoromethoxy)pyrazin-2- yl)amino)cyclopentyl)amino)pyridin- 3-yl)-1,8-naphthyridin-2(1 H)-one |
| 88 | 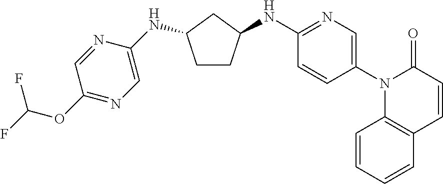 [SMILES: O=c1ccc2ccccc2n1-c1ccc(N[C@H]2CC[C@H](Nc3cnc(OC(F)F)cn3)C2)nc1] | 1-(6-(((1S,3S)-3-((5- (Difluoromethoxy)pyrazin-2- yl)amino)cyclopentyl)amino)pyridin- 3-yl)quinolin-2(1H)-one |
| 89 | 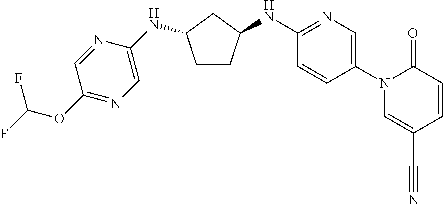 [SMILES: N#Cc1ccc(=O)n(-c2ccc(N[C@H]3CC[C@H](Nc4cnc(OC(F)F)cn4)C3)nc2)c1] | 6′-(((1S,3S)-3-((5- (Difluoromethoxy)pyrazin-2- yl)amino)cyclopentyl)amino)-2-oxo- 2H-[1,3′-bipyridine]-5-carbonitrile |
| 90 | 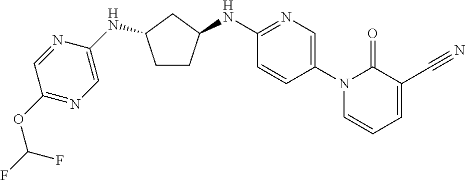 [SMILES: N#Cc1cccn(-c2ccc(N[C@H]3CC[C@H](Nc4cnc(OC(F)F)cn4)C3)nc2)c1=O] | 6′-(((1S,3S)-3-((5- (Difluoromethoxy)pyrazin-2- yl)amino)cyclopentyl)amino)-2-oxo- 2H-[1,3′-bipyridine]-3-carbonitrile |
| 91 | 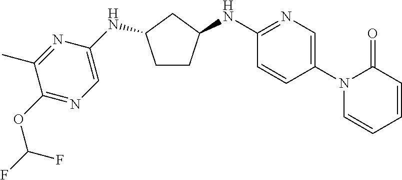 [SMILES: Cc1nc(N[C@H]2CC[C@H](Nc3ccc(-n4ccccc4=O)cn3)C2)cnc1OC(F)F] | 6′-(((1S,3S)-3-((5-(Difluoromethoxy)- 6-methylpyrazin-2- yl)amino)cyclopentyl)amino)-2H- [1,3′-bipyridin]-2-one |
| 92 | 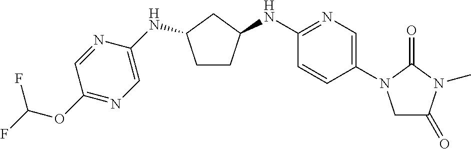 [SMILES: CN1C(=O)CN(c2ccc(N[C@H]3CC[C@H](Nc4cnc(OC(F)F)cn4)C3)nc2)C1=O] | 1-(6-(((1S,3S)-3-((5- (Difluoromethoxy)pyrazin-2- yl)amino)cyclopentyl)amino)pyridin- 3-yl)-3-methylimidazolidine-2,4-dione |
| 93 | 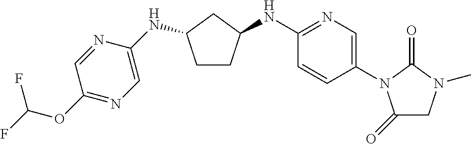 [SMILES: CN1CC(=O)N(c2ccc(N[C@H]3CC[C@H](Nc4cnc(OC(F)F)cn4)C3)nc2)C1=O] | 3-(6-(((1S,3S)-3-((5- (Difluoromethoxy)pyrazin-2- yl)amino)cyclopentyl)amino)pyridin- 3-yl)-1-methylimidazolidine-2,4-dione |
| 94 | 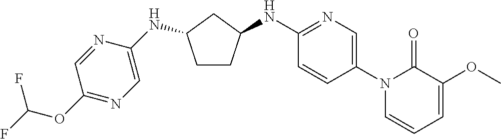 [SMILES: COc1cccn(-c2ccc(N[C@H]3CC[C@H](Nc4cnc(OC(F)F)cn4)C3)nc2)c1=O] | 6′-(((1S,3S)-3-((5- (Difluoromethoxy)pyrazin-2- yl)amino)cyclopentyl)amino)-3- methoxy-2H-[1,3′-bipyridin]-2-one |
| 95 | 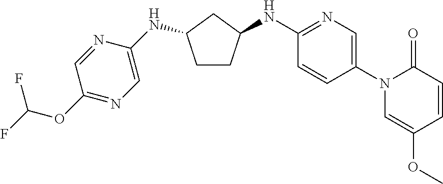 [SMILES: COc1ccc(=O)n(-c2ccc(N[C@H]3CC[C@H](Nc4cnc(OC(F)F)cn4)C3)nc2)c1] | 6′-(((1S,3S)-3-((5- (Difluoromethoxy)pyrazin-2- yl)amino)cyclopentyl)amino)-5- methoxy-2H-[1,3′-bipyridin]-2-one |
| 96 | 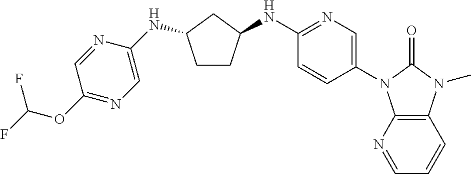 [SMILES: Cn1c(=O)n(-c2ccc(N[C@H]3CC[C@H](Nc4cnc(OC(F)F)cn4)C3)nc2)c2ncccc21] | 3-(6-(((1S,3S)-3-((5- (Difluoromethoxy)pyrazin-2- yl)amino)cyclopentyl)amino)pyridin- 3-yl)-1-methyl-1,3-dihydro-2H- imidazo[4,5-b]pyridin-2-one |
| 97 | 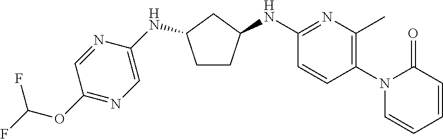 [SMILES: Cc1nc(N[C@H]2CC[C@H](Nc3cnc(OC(F)F)cn3)C2)ccc1-n1ccccc1=O] | 6′-(((1S,3S)-3-((5- (Difluoromethoxy)pyrazin-2- yl)amino)cyclopentyl)amino)-2′- methyl-2H-[1,3′-bipyridin]-2-one |
| 98 | 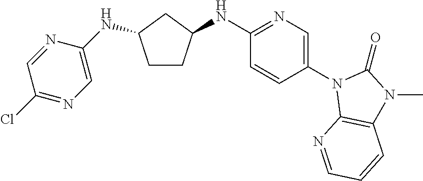 [SMILES: Cn1c(=O)n(-c2ccc(N[C@H]3CC[C@H](Nc4cnc(Cl)cn4)C3)nc2)c2ncccc21] | 3-(6-(((1S,3S)-3-((5-Chloropyrazin-2- yl)amino)cyclopentyl)amino)pyridin- 3-yl)-1-methyl-1,3-dihydro-2H- imidazo[4,5-b]pyridin-2-one |
| 99 | 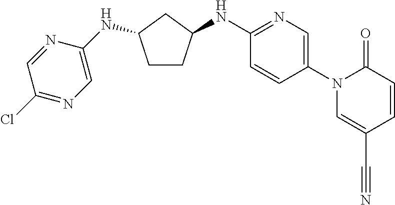 [SMILES: N#Cc1ccc(=O)n(-c2ccc(N[C@H]3CC[C@H](Nc4cnc(Cl)cn4)C3)nc2)c1] | 6′-(((1S,3S)-3-((5-Chloropyrazin-2- yl)amino)cyclopentyl)amino)-2-oxo- 2H-[1,3′-bipyridine]-5-carbonitrile |
| 100 | 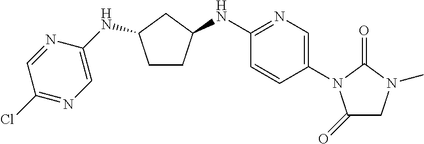 [SMILES: CN1CC(=O)N(c2ccc(N[C@H]3CC[C@H](Nc4cnc(Cl)cn4)C3)nc2)C1=O] | 3-(6-(((1S,3S)-3-((5-Chloropyrazin-2- yl)amino)cyclopentyl)amino)pyridin- 3-yl)-1-methylimidazolidine-2,4-dione |
| 101 | 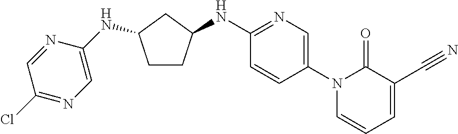 [SMILES: N#Cc1cccn(-c2ccc(N[C@H]3CC[C@H](Nc4cnc(Cl)cn4)C3)nc2)c1=O] | 6′-(((1S,3S)-3-((5-Chloropyrazin-2- yl)amino)cyclopentyl)amino)-2-oxo- 2H-[1,3′-bipyridine]-3-carbonitrile |
| 102 | 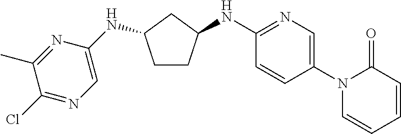 [SMILES: Cc1nc(N[C@H]2CC[C@H](Nc3ccc(-n4ccccc4=O)cn3)C2)cnc1Cl] | 6′-(((1S,3S)-3-((5-Chloro-6- methylpyrazin-2- yl)amino)cyclopentyl)amino)-2H- [1,3′-bipyridin]-2-one |
| 103 | 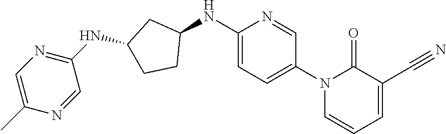 [SMILES: Cc1cnc(N[C@H]2CC[C@H](Nc3ccc(-n4cccc(C#N)c4=O)cn3)C2)cn1] | 6′-(((1S,3S)-3-((5-Methylpyrazin-2- yl)amino)cyclopentyl)amino)-2-oxo- 2H-[1,3′-bipyridine]-3-carbonitrile |
| 104 | 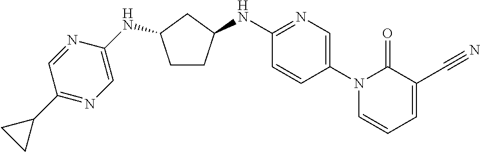 [SMILES: N#Cc1cccn(-c2ccc(N[C@H]3CC[C@H](Nc4cnc(C5CC5)cn4)C3)nc2)c1=O] | 6′-(((1S,3S)-3-((5- Cyclopropylpyrazin-2- yl)amino)cyclopentyl)amino)-2-oxo- 2H-[1,3′-bipyridine]-3-carbonitrile |
| 105 | 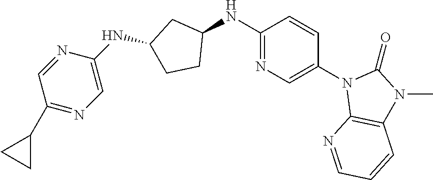 [SMILES: Cn1c(=O)n(-c2ccc(N[C@H]3CC[C@H](Nc4cnc(C5CC5)cn4)C3)nc2)c2ncccc21] | 3-(6-(((1S,3S)-3-((5- Cyclopropylpyrazin-2- yl)amino)cyclopentyl)amino)pyridin- 3-yl)-1-methyl-1,3-dihydro-2H- imidazo[4,5-b]pyridin-2-one |
| 106 | 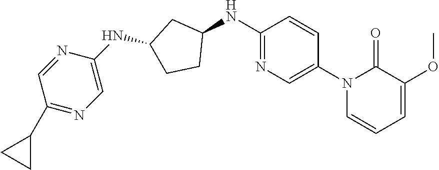 [SMILES: COc1cccn(-c2ccc(N[C@H]3CC[C@H](Nc4cnc(C5CC5)cn4)C3)nc2)c1=O] | 6′-(((1S,3S)-3-((5- Cyclopropylpyrazin-2- yl)amino)cyclopentyl)amino)-3- methoxy-2H-[1,3′-bipyridin]-2-one |
| 107 | 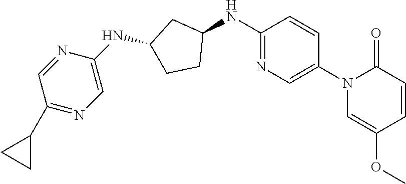 [SMILES: COc1ccc(=O)n(-c2ccc(N[C@H]3CC[C@H](Nc4cnc(C5CC5)cn4)C3)nc2)c1] | 6′-(((1S,3S)-3-((5- Cyclopropylpyrazin-2- yl)amino)cyclopentyl)amino)-5- methoxy-2H-[1,3′-bipyridin]-2-one |
| 108 | 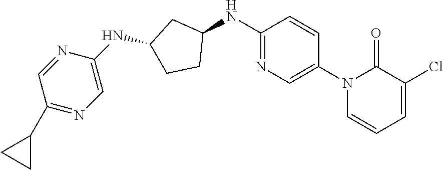 [SMILES: O=c1c(Cl)cccn1-c1ccc(N[C@H]2CC[C@H](Nc3cnc(C4CC4)cn3)C2)nc1] | 3-Chloro-6′-(((1S,3S)-3-((5- cyclopropylpyrazin-2- yl)amino)cyclopentyl)amino)-2H- [1,3′-bipyridin]-2-one |
| 109 | 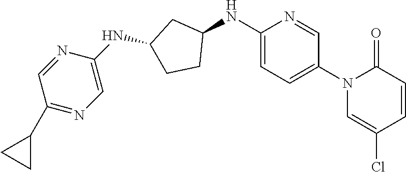 [SMILES: O=c1ccc(Cl)cn1-c1ccc(N[C@H]2CC[C@H](Nc3cnc(C4CC4)cn3)C2)nc1] | 5-Chloro-6′-(((1S,3S)-3-((5- Cyclopropylpyrazin-2- yl)amino)cyclopentyl)amino)-2H- [1,3′-bipyridin]-2-one |
| 110 | 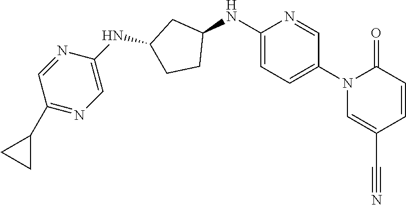 [SMILES: N#Cc1ccc(=O)n(-c2ccc(N[C@H]3CC[C@H](Nc4cnc(C5CC5)cn4)C3)nc2)c1] | 6′-(((1S,3S)-3-((5- Cyclopropylpyrazin-2- yl)amino)cyclopentyl)amino)-2-oxo- 2H-[1,3′-bipyridine]-5-carbonitrile |
| 111 | 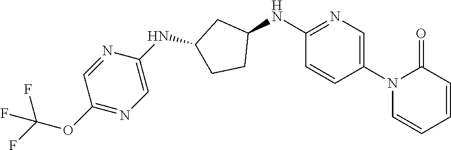 [SMILES: O=c1ccccn1-c1ccc(N[C@H]2CC[C@H](Nc3cnc(OC(F)(F)F)cn3)C2)nc1] | 6′-(((1S,3S)-3-((5- (Trifluoromethoxy)pyrazin-2- yl)amino)cyclopentyl)amino)-2H- [1,3′-bipyridin]-2-one |
| 139 | 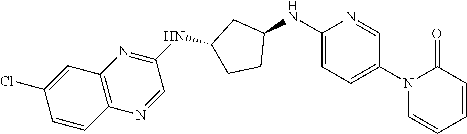 [SMILES: O=c1ccccn1-c1ccc(N[C@H]2CC[C@H](Nc3cnc4ccc(Cl)cc4n3)C2)nc1] | 6′-(((1S,3S)-3-((7-Chloroquinoxalin- 2-yl)amino)cyclopentyl)amino)-2H- [1,3′-bipyridin]-2-one |
| 140 | 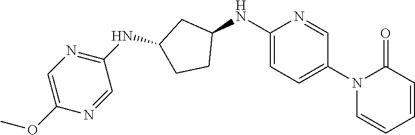 [SMILES: COc1cnc(N[C@H]2CC[C@H](Nc3ccc(-n4ccccc4=O)cn3)C2)cn1] | 6′-(((1S,3S)-3-((5-Methoxypyrazin-2- yl)amino)cyclopentyl)amino)-2H- [1,3′-bipyridin]-2-one |
| 141 | 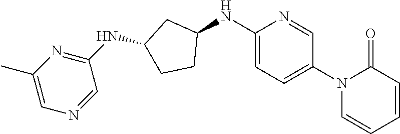 [SMILES: Cc1cncc(N[C@H]2CC[C@H](Nc3ccc(-n4ccccc4=O)cn3)C2)n1] | 6′-(((1S,3S)-3-((6-Methylpyrazin-2- yl)amino)cyclopentyl)amino)-2H- [1,3′-bipyridin]-2-one |
| 142 | 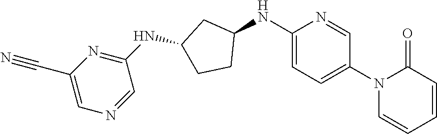 [SMILES: N#Cc1cncc(N[C@H]2CC[C@H](Nc3ccc(-n4ccccc4=O)cn3)C2)n1] | 6-(((1S,3S)-3-((2-Oxo-2H-[1,3′- bipyridin]-6′- yl)amino)cyclopentyl)amino)pyrazine- 2-carbonitrile |
| 143 | 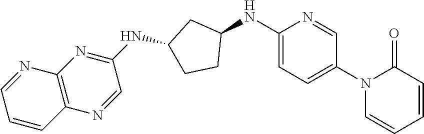 [SMILES: O=c1ccccn1-c1ccc(N[C@H]2CC[C@H](Nc3cnc4cccnc4n3)C2)nc1] | 6′-(((1S,3S)-3-(Pyrido[2,3-b]pyrazin- 3-ylamino)cyclopentyl)amino)-2H- [1,3′-bipyridin]-2-one |
| 144 | 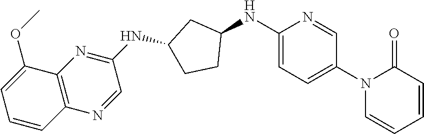 [SMILES: COc1cccc2ncc(N[C@H]3CC[C@H](Nc4ccc(-n5ccccc5=O)cn4)C3)nc12] | 6′-(((1S,3S)-3-((8- Methoxyquinoxalin-2- yl)amino)cyclopentyl)amino)-2H- [1,3′-bipyridin]-2-one |
| 145 | 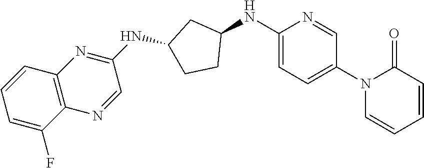 [SMILES: O=c1ccccn1-c1ccc(N[C@H]2CC[C@H](Nc3cnc4c(F)cccc4n3)C2)nc1] | 6′-(((1S,3S)-3-((5-Fluoroquinoxalin-2- yl)amino)cyclopentyl)amino)-2H- [1,3′-bipyridin]-2-one |
| 146 | 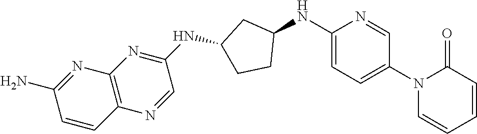 [SMILES: Nc1ccc2ncc(N[C@H]3CC[C@H](Nc4ccc(-n5ccccc5=O)cn4)C3)nc2n1] | 6′-(((1S,3S)-3-((6-Aminopyrido[2,3- b]pyrazin-3- yl)amino)cyclopentyl)amino)-2H- [1,3′-bipyridin]-2-one |
| 147 | 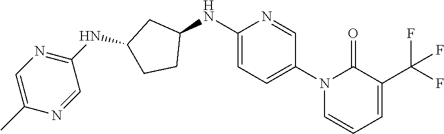 [SMILES: Cc1cnc(N[C@H]2CC[C@H](Nc3ccc(-n4cccc(C(F)(F)F)c4=O)cn3)C2)cn1] | 6′-(((1S,3S)-3-((5-Methylpyrazin-2- yl)amino)cyclopentyl)amino)-3- (trifluoromethyl)-2H-[1,3′-bipyridin]- 2-one |
| 148 | 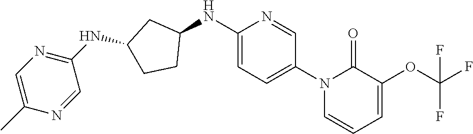 [SMILES: Cc1cnc(N[C@H]2CC[C@H](Nc3ccc(-n4cccc(OC(F)(F)F)c4=O)cn3)C2)cn1] | 6′-(((1S,3S)-3-((5-Methylpyrazin-2- yl)amino)cyclopentyl)amino)-3- (trifluoromethoxy)-2H-[1,3′- bipyridin]-2-one |

In some embodiments the compound is selected from 36, 47, and 103. In some embodiments the compound is 36.

Further compounds are shown in Table 2a:

TABLE 2a

| Ex | Structure | Name |
|---|---|---|
| 149 | 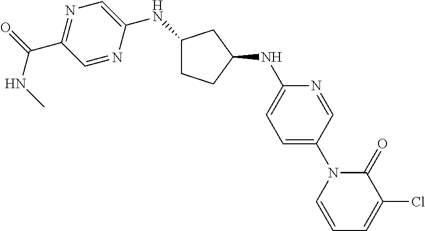 [SMILES: CNC(=O)c1cnc(N[C@H]2CC[C@H](Nc3ccc(-n4cccc(Cl)c4=O)cn3)C2)cn1] | 5-(((1S,3S)-3-((3-Chloro-2-oxo-2H- [1,3′-bipyridin]-6′- yl)amino)cyclopentyl)amino)-N- methylpyrazine-2-carboxamide |
| 150 | 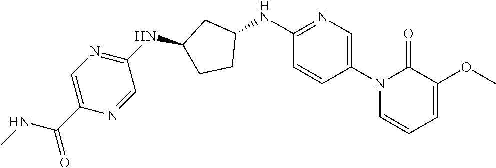 | 5-(((1S,3S)-3-((3-Methoxy-2-oxo- 2H-[1,3′-bipyridin]-6′- yl)amino)cyclopentyl)amino)-N- methylpyrazine-2-carboxamide |
| 162 | 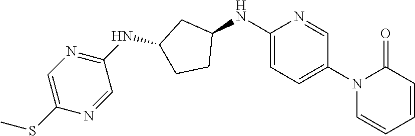 [SMILES: CSc1cnc(N[C@H]2CC[C@H](Nc3ccc(-n4ccccc4=O)cn3)C2)cn1] | 6′-(((1S,3S)-3-((5- (Methylthio)pyrazin-2- yl)amino)cyclopentyl)amino)-2H- [1,3′-bipyridin]-2-one |
| 163 | 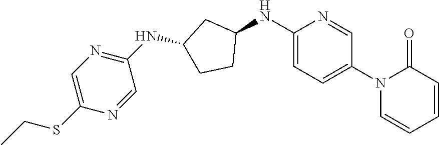 [SMILES: CCSc1cnc(N[C@H]2CC[C@H](Nc3ccc(-n4ccccc4=O)cn3)C2)cn1] | 6′-(((1S,3S)-3-((5-(Ethylthio)pyrazin- 2-yl)amino)cyclopentyl)amino)-2H- [1,3′-bipyridin]-2-one |
| 164 | 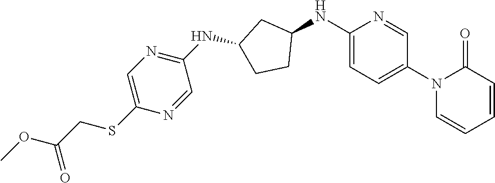 [SMILES: COC(=O)CSc1cnc(N[C@H]2CC[C@H](Nc3ccc(-n4ccccc4=O)cn3)C2)cn1] | Methyl 2-((5-(((1S,3S)-3-((2-oxo-2H- [1,3′-bipyridin]-6′- yl)amino)cyclopentyl)amino)pyrazin- 2-yl)thio)acetate |
| 170 | 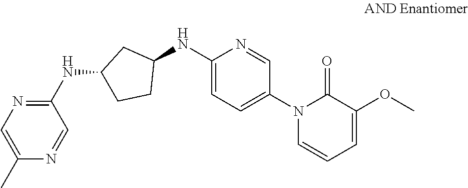 [SMILES: COc1cccn(-c2ccc(N[C@H]3CC[C@H](Nc4cnc(C)cn4)C3)nc2)c1=O] | rac-3-Methoxy-6′-(((1R,3R)-3-((5- methylpyrazin-2- yl)amino)cyclopentyl)amino)-2H- [1,3′-bipyridin]-2-one |
| 171 | 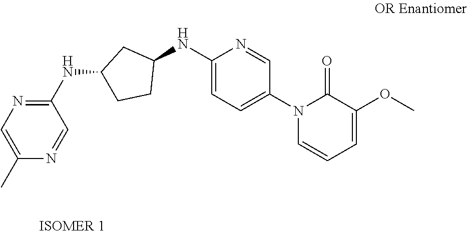 [SMILES: COc1cccn(-c2ccc(N[C@H]3CC[C@H](Nc4cnc(C)cn4)C3)nc2)c1=O] | rel-3-Methoxy-6′-(((1R,3R)-3-((5- methylpyrazin-2- yl)amino)cyclopentyl)amino)-2H- [1,3′-bipyridin]-2-one, Isomer 1 |
| 172 | 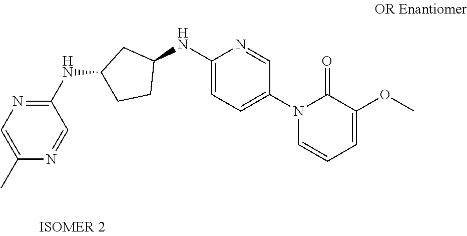 [SMILES: COc1cccn(-c2ccc(N[C@H]3CC[C@H](Nc4cnc(C)cn4)C3)nc2)c1=O] | rel-3-Methoxy-6′-(((1R,3R)-3-((5- methylpyrazin-2- yl)amino)cyclopentyl)amino)-2H- [1,3′-bipyridin]-2-one, Isomer 2 |
| 176 | 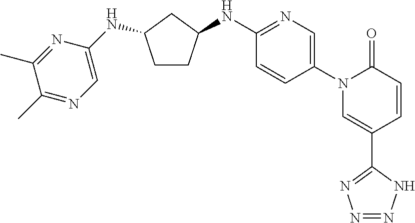 [SMILES: Cc1ncc(N[C@H]2CC[C@H](Nc3ccc(-n4cc(-c5nnn[nH]5)ccc4=O)cn3)C2)nc1C] | 6′-(((1S,3S)-3-((5,6- Dimethylpyrazin-2- yl)amino)cyclopentyl)amino)-5-(1H- tetrazol-5-yl)-2H-[1,3′-bipyridin]-2- one |
| 177 | 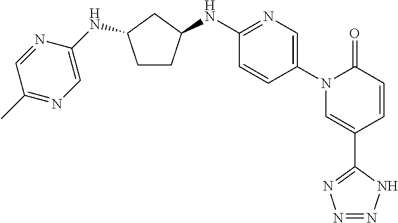 [SMILES: Cc1cnc(N[C@H]2CC[C@H](Nc3ccc(-n4cc(-c5nnn[nH]5)ccc4=O)cn3)C2)cn1] | 6′-(((1S,3S)-3-((5-Methylpyrazin-2- yl)amino)cyclopentyl)amino)-5-(1H- tetrazol-5-yl)-2H-[1,3′-bipyridin]-2- one |
| 178 | 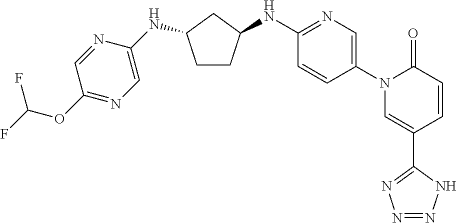 [SMILES: O=c1ccc(-c2nnn[nH]2)cn1-c1ccc(N[C@H]2CC[C@H](Nc3cnc(OC(F)F)cn3)C2)nc1] | 6′-(((1S,3S)-3-((5- (Difluoromethoxy)pyrazin-2- yl)amino)cyclopentyl)amino)-5-(1H- tetrazol-5-yl)-2H-[1,3′-bipyridin]-2- one |
| 179 | 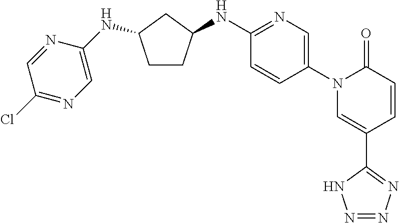 [SMILES: O=c1ccc(-c2nnn[nH]2)cn1-c1ccc(N[C@H]2CC[C@H](Nc3cnc(Cl)cn3)C2)nc1] | 6′-(((1S,3S)-3-((5-Chloropyrazin-2- yl)amino)cyclopentyl)amino)-5-(1H- tetrazol-5-yl)-2H-[1,3′-bipyridin]-2- one |
| 203 | 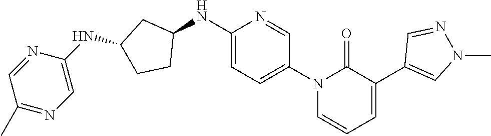 | 3-(1-Methyl-1H-pyrazol-4-yl)-6′- (((1S,3S)-3-((5-methylpyrazin-2- yl)amino)cyclopentyl)amino)-2H- [1,3′-bipyridin]-2-one |
| 204 | 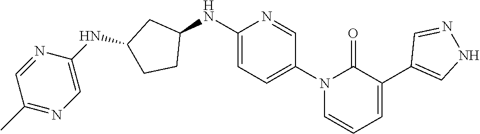 [SMILES: Cc1cnc(N[C@H]2CC[C@H](Nc3ccc(-n4cccc(-c5cn[nH]c5)c4=O)cn3)C2)cn1] | 6′-(((1S,3S)-3-((5-Methylpyrazin-2- yl)amino)cyclopentyl)amino)-3-(1H- pyrazol-4-yl)-2H-[1,3′-bipyridin]-2- one |
| 205 | 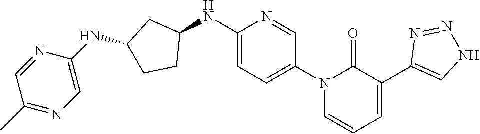 [SMILES: Cc1cnc(N[C@H]2CC[C@H](Nc3ccc(-n4cccc(-c5c[nH]nn5)c4=O)cn3)C2)cn1] | 6′-(((1S,3S)-3-((5-Methylpyrazin-2- yl)amino)cyclopentyl)amino)-3-(1H- 1,2,3-triazol-4-yl)-2H-[1,3′- bipyridin]-2-one |
| 206 | 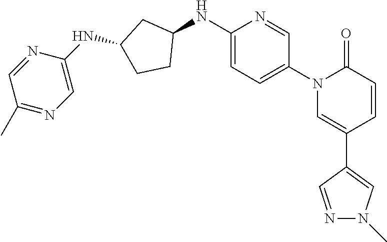 | 5-(1-Methyl-1H-pyrazol-4-yl)-6′- (((1S,3S)-3-((5-methylpyrazin-2- yl)amino)cyclopentyl)amino)-2H- [1,3′-bipyridin]-2-one |
| 207 | 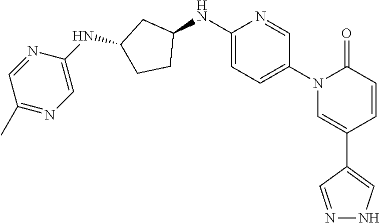 [SMILES: Cc1cnc(N[C@H]2CC[C@H](Nc3ccc(-n4cc(-c5cn[nH]c5)ccc4=O)cn3)C2)cn1] | 6′-(((1S,3S)-3-((5-Methylpyrazin-2- yl)amino)cyclopentyl)amino)-5-(1H- pyrazol-4-yl)-2H-[1,3′-bipyridin]-2- one |
| 209 | 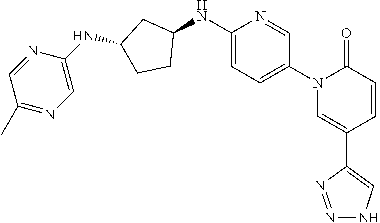 [SMILES: Cc1cnc(N[C@H]2CC[C@H](Nc3ccc(-n4cc(-c5c[nH]nn5)ccc4=O)cn3)C2)cn1] | 6′-(((1S,3S)-3-((5-Methylpyrazin-2- yl)amino)cyclopentyl)amino)-5-(1H- 1,2,3-triazol-4-yl)-2H-[1,3′- bipyridin]-2-one |
| 215 | 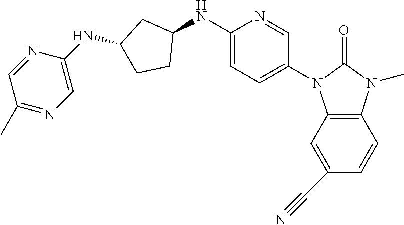 | 1-Methyl-3-(6-(((1S,3S)-3-((5- methylpyrazin-2- yl)amino)cyclopentyl)amino)pyridin- 3-yl)-2-oxo-2,3-dihydro-1H- benzo[d]imidazole-5-carbonitrile |
| 216 | 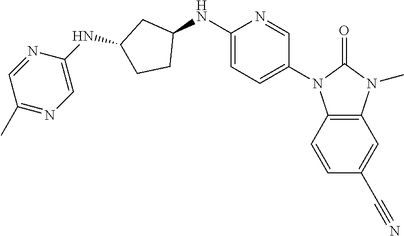 | 3-Methyl-1-(6-(((1S,3S)-3-((5- methylpyrazin-2- yl)amino)cyclopentyl)amino)pyridin- 3-yl)-2-oxo-2,3-dihydro-1H- benzo[d]imidazole-5-carbonitrile |
| 218 | 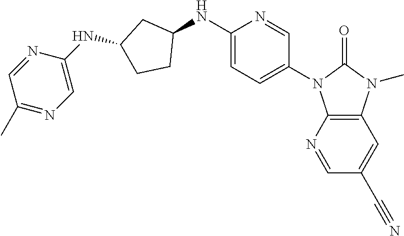 | 1-Methyl-3-(6-(((1S,3S)-3-((5- methylpyrazin-2- yl)amino)cyclopentyl)amino)pyridin- 3-yl)-2-oxo-2,3-dihydro-1H- imidazo[4,5-b]pyridine-6-carbonitrile |
| 226 | 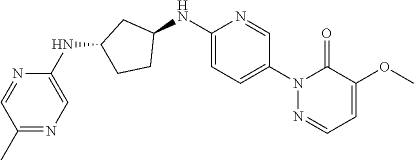 | 4-Methoxy-2-(6-(((1S,3S)-3-((5- methylpyrazin-2- yl)amino)cyclopentyl)amino)pyridin- 3-yl)pyridazin-3(2H)-one |
| 227 | 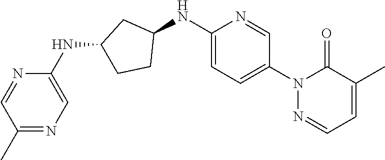 | 4-Methyl-2-(6-(((1S,3S)-3-((5- methylpyrazin-2- yl)amino)cyclopentyl)amino)pyridin- 3-yl)pyridazin-3(2H)-one |
| 231 | 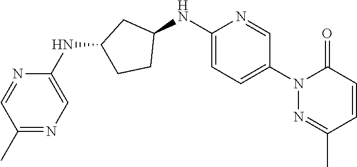 [SMILES: Cc1cnc(N[C@H]2CC[C@H](Nc3ccc(-n4nc(C)ccc4=O)cn3)C2)cn1] | 6-Methyl-2-(6-(((1S,3S)-3-((5- methylpyrazin-2- yl)amino)cyclopentyl)amino)pyridin- 3-yl)pyridazin-3(2H)-one |
| 232 | 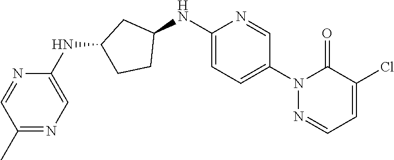 [SMILES: Cc1cnc(N[C@H]2CC[C@H](Nc3ccc(-n4nccc(Cl)c4=O)cn3)C2)cn1] | 4-Chloro-2-(6-(((1S,3S)-3-((5- methylpyrazin-2- yl)amino)cyclopentyl)amino)pyridin- 3-yl)pyridazin-3(2H)-one |
| 233 | 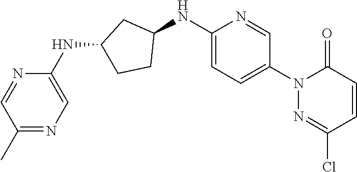 [SMILES: Cc1cnc(N[C@H]2CC[C@H](Nc3ccc(-n4nc(Cl)ccc4=O)cn3)C2)cn1] | 6-Chloro-2-(6-(((1S,3S)-3-((5- methylpyrazin-2- yl)amino)cyclopentyl)amino)pyridin- 3-yl)pyridazin-3(2H)-one |
| 252 | 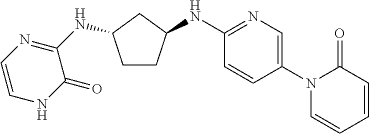 [SMILES: O=c1[nH]ccnc1N[C@H]1CC[C@H](Nc2ccc(-n3ccccc3=O)cn2)C1] | 6′-(((1S,3S)-3-((3-Oxo-3,4- dihydropyrazin-2- yl)amino)cyclopentyl)amino)-2H- [1,3′-bipyridin]-2-one |
| 253 | 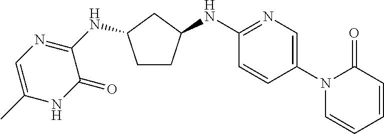 [SMILES: Cc1cnc(N[C@H]2CC[C@H](Nc3ccc(-n4ccccc4=O)cn3)C2)c(=O)[nH]1] | 6′-(((1S,3S)-3-((5-Methyl-3-oxo-3,4- dihydropyrazin-2- yl)amino)cyclopentyl)amino)-2H- [1,3′-bipyridin]-2-one |
| 259 | 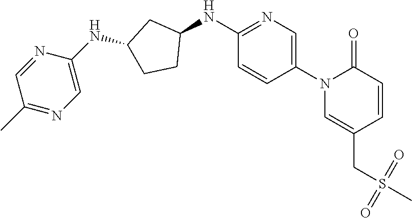 | 6′-(((1S,3S)-3-((5-Methylpyrazin-2- yl)amino)cyclopentyl)amino)-5- ((methylsulfonyl)methyl)-2H-[1,3′- bipyridin]-2-one |
| 274 | 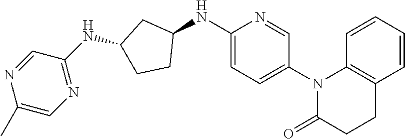 [SMILES: Cc1cnc(N[C@H]2CC[C@H](Nc3ccc(N4C(=O)CCc5ccccc54)cn3)C2)cn1] | 1-(6-(((1S,3S)-3-((5-Methylpyrazin- 2- yl)amino)cyclopentyl)amino)pyridin- 3-yl)-3,4-dihydroquinolin-2(1H)-one |
| 276 | 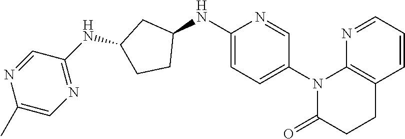 [SMILES: Cc1cnc(N[C@H]2CC[C@H](Nc3ccc(N4C(=O)CCc5cccnc54)cn3)C2)cn1] | 1-(6-(((1S,3S)-3-((5-Methylpyrazin- 2- yl)amino)cyclopentyl)amino)pyridin- 3-yl)-3,4-dihydro-1,8-naphthyridin- 2(1H)-one |
| 278 | 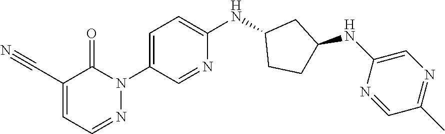 [SMILES: Cc1cnc(N[C@H]2CC[C@H](Nc3ccc(-n4nccc(C#N)c4=O)cn3)C2)cn1] | 2-(6-(((1S,3S)-3-((5-Methylpyrazin- 2- yl)amino)cyclopentyl)amino)pyridin- 3-yl)-3-oxo-2,3-dihydropyridazine-4- carbonitrile |
| 282 | 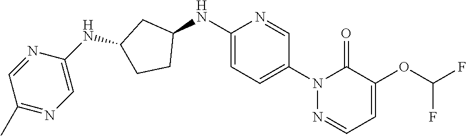 [SMILES: Cc1cnc(N[C@H]2CC[C@H](Nc3ccc(-n4nccc(OC(F)F)c4=O)cn3)C2)cn1] | 4-(Difluoromethoxy)-2-(6-(((1S,3S)- 3-((5-methylpyrazin-2- yl)amino)cyclopentyl)amino)pyridin- 3-yl)pyridazin-3(2H)-one |

General Synthesis

The compounds according to general formula (I-B) can be prepared according to the following schemes 1, 2 or 3. The schemes and procedures described below illustrate synthetic routes to the compounds of general formula (I-B) and are not intended to be limiting. It is clear that the order of transformations as exemplified in schemes 1, 2 or 3 can be modified in various ways. The order of transformations exemplified in these schemes is therefore not intended to be limiting.

Routes for the preparation of compounds of general formula (I-B) and corresponding intermediates are described in schemes 1, 2 or 3.

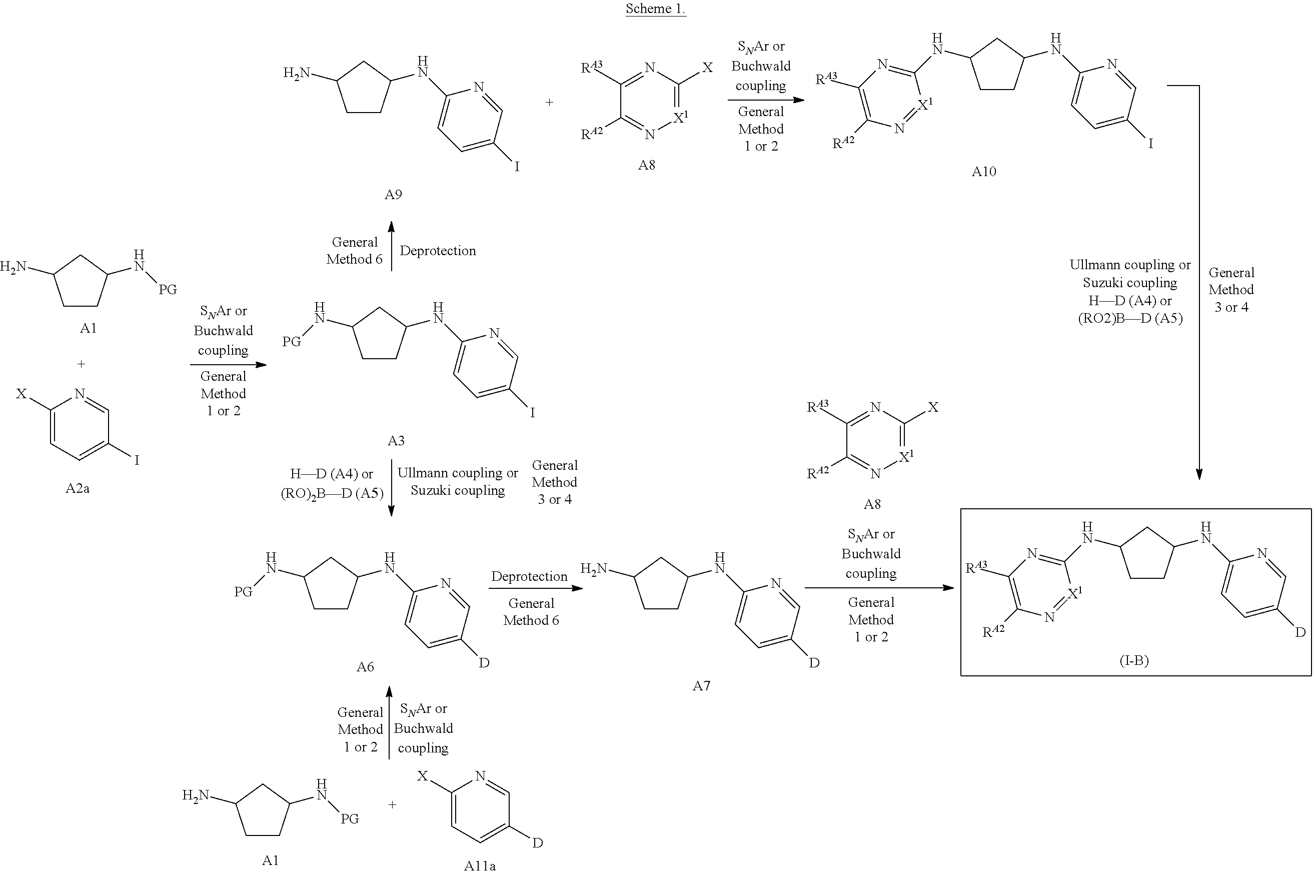

Scheme 1: Routes for the preparation of compounds of general formula (I-B) in which X is a leaving group, PG is a protective group and D, X¹, R^{A2} and R^{A3} have the meaning as given for general formula (I-B), supra.

Monoarylated diamines of general formula (A3) can be obtained via nucleophilic aromatic substitution (S_{N}Ar) or palladium catalyzed Buchwald-Hartwig amination between monoprotected diamines (A1) or their corresponding salts and heteroaryls (A2a) with X being a leaving group like halogen or —S(O)Me as depicted in Scheme 1. For S_{N}Ar approaches with X being groups like for example fluorine or —S(O)Me, diamines (A1) may be reacted with (A2a) in the presence of inorganic bases like K₂CO₃ or Na₂CO₃ or in the presence of organic bases like triethylamine or DIPEA or without any additional base in polar solvents such as for example DMSO, NMP or nBuOH at temperatures between 100-130° C. The reaction times may vary between 1 hour and 24 hours. In certain instances, it can be beneficial to apply microwave heating.

For palladium catalyzed Buchwald-Hartwig aminations all methods that are known in the art may be applied. For example, diamines (A1) may be reacted with (A2a) in the presence of a palladium catalyst like Pd PEPPSI-lpentCl [CAS 1612891-29-8], Pd₂(dba)₃ or tBuXPhos Pd G3 [1447963-75-8] and a base like Cs₂CO₃ or NaOtBu in aprotic solvents like 1,4-dioxane, DMF, toluene or DMA at temperatures between room temperature and 130° C., preferably at 65-100° C., for 15-24 h.

Diamines of general formula (A1) and heteroaryls of general formula (A2a) are either commercially available or can be prepared according to procedures available from the public domain. For the synthesis of diamines (A1) see for example WO2004004726 and references therein.

Arylated diamines of general formula (A6) can be obtained from (A3) via copper catalyzed Ullmann couplings with heterocycles (A4) or via palladium catalyzed Suzuki couplings with boronic acid derivatives (A5). For Ullmann couplings all methods that are known in the art may be applied. For example, (A3) may be reacted with (A4) in the presence of a copper catalyst like Cu(I)I, Cu(OTf)₂ or Cu(OAc)₂ and a base like Cs₂CO₃ or K₂CO₃ in polar, aprotic solvents like 1,4-dioxane, DMF or pyridine at temperatures between room temperature and 120° C., preferably at 100° C. for 15-20 h. In some instances a ligand like N¹,N2-dimethylcyclohexane-1,2-diamine, TMEDA, N¹,N²-dimethylethane-1,2-diamine or N,N-dimethylglycine might be added to the reaction mixture.

For Suzuki couplings towards (A6) all methods that are known in the art may be applied. For example, (A3) may be reacted with boronic acid derivatives (A5) in the presence of a palladium catalyst like 1,1′-bis(di-tert-butylphosphino)ferrocene palladium dichloride [CAS 95408-45-0] or 1,1′-bis(diphenylphosphino)ferrocene palladium dichloride [CAS 72287-26-4] and a base like Cs₂CO₃, K₂CO₃ or K₃PO₄ in polar solvents such as 1,4-dioxane, THF and water or mixtures thereof at temperatures between room temperature and 120° C. for 2-15 hours.

Heterocycles of general formula (A4) and boronic acid derivatives of general formula (A5) are either commercially available or can be prepared according to procedures available from the public domain.

Primary amines of general formula (A7) can be obtained from monoprotected diamines of general formula (A6) via deprotection methods. Depending on the protective group applied these can be for example acidic, basic, oxidative or hydrogenation methods. Appropriate protective moieties for amino groups and their introduction and cleavage are well-known in the art. For an overview of protective group chemistry see for example Wuts 2014.

Final compounds of general formula (I-B) can be synthesized from primary amines of general formula (A7) via nucleophilic aromatic substitution (S_{N}Ar) or palladium catalyzed Buchwald-Hartwig amination. Primary amines of general formula (A7) can be reacted with heteroaryls of general formula (A8) with X being a leaving group like halogen such as chlorine or —S(O)Me applying procedures in analogy to those described for the synthesis of (A3) from (A1) and (A2a) in Scheme 1. Heteroaryls of general formula (A8) are either commercially available or can be prepared according to procedures available from the public domain.

An alternative route to compounds of general formula (I-B) starts with deprotection of diamines of general formula (A3) to give primary amines of general formula (A9) as depicted in Scheme 1. For deprotection the same procedures apply as described for the synthesis of (A7) from (A6).

Primary amines of general formula (A9) in turn can be reacted with heteroaryls of general formula (A8) via nucleophilic aromatic substitution (S_{N}Ar) or palladium catalyzed Buchwald-Hartwig amination to give aryl iodides of general formula (A10) applying procedures in analogy to those described for the synthesis of (A3) from (A1) and (A2a) in Scheme 1.

Final compounds of general formula (I-B) can be synthesized from aryl iodides of general formula (10) via copper catalyzed Ullmann couplings with heterocycles H-D (A4) or via palladium catalyzed Suzuki couplings with boronic acid derivatives (A5) applying procedures in analogy to those described for the synthesis of compounds (A6) from (A3) in Scheme 1.

Yet another approach to compounds of general formula (I-B) starts from monoprotected diamines (1) or their corresponding salts and preassembled heteroaryls (A11a) with X being a leaving group like halogen or —S(O)Me via nucleophilic aromatic substitution (S_{N}Ar) or palladium catalyzed Buchwald-Hartwig amination to give arylated diamines of general formula (A6). The procedures that can be applied are in analogy to those described for the synthesis of (A3) from (A1) and (A2a) in Scheme 1. Heteroaryls of general formula (A11a) are either commercially available or can be prepared according to procedures available from the public domain (for example via Chan-Lam coupling). Specific examples of (A11a) are described in the subsequent paragraphs.

An alternative route for the preparation of compounds of general formula (I-B) and intermediates of general formula (A10) is depicted in Scheme 2.

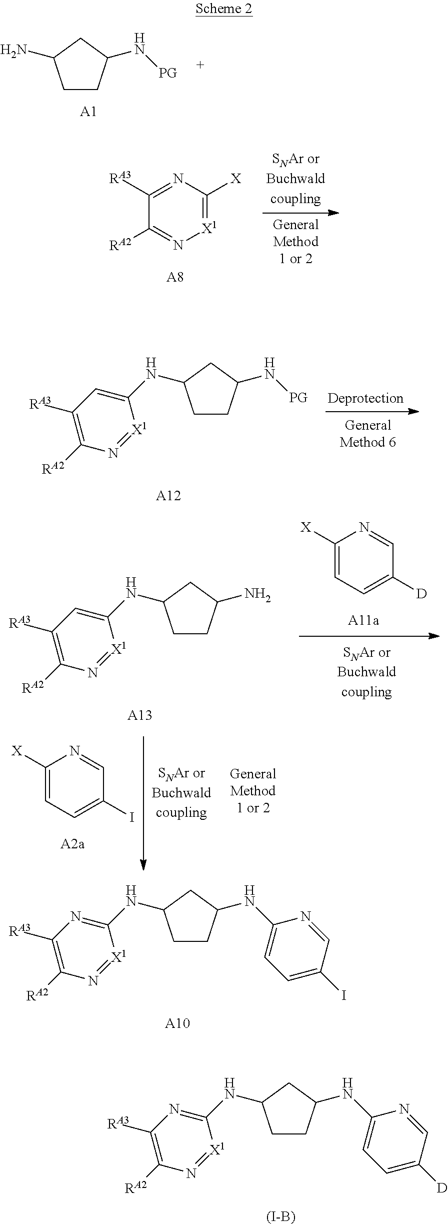

Scheme 2: Routes for the preparation of compounds of general formula (I-B) and intermediates (A10) in which X is a leaving group, PG is a protective group and D, X¹, R^{A} and R^{A3} have the meaning as given for general formula (I-B), supra.

Monoarylated diamines of general formula (A12) can be obtained via nucleophilic aromatic substitution (S_{N}Ar) or palladium catalyzed Buchwald-Hartwig amination between monoprotected diamines (A1) or their corresponding salts and heteroaryls (A8) with X being a leaving group such as halogen or —S(O)Me. The procedures that can be applied are in analogy to those described for the synthesis of (A3) from (A1) and (A2a) in Scheme 1.

Deprotection of diamines of general formula (A12) can give primary amines of general formula (A13). For deprotection the same procedures apply as described for the synthesis of (A7) from (A6) in Scheme 1.

Final compounds of general formula (I-B) in turn can be synthesized from primary amines (A13) or their corresponding salts and preassembled heteroaryls (A11a) with X being a leaving group like halogen or —S(O)Me via nucleophilic aromatic substitution (S_{N}Ar) or palladium catalyzed Buchwald-Hartwig amination. The procedures that can be applied are in analogy to those described for the synthesis of (A6) from (A1) and (A11a) in Scheme 1.

For the synthesis of intermediates of general formula (A10) primary amines (A13) or their corresponding salts may be reacted with heteroaryls (A2a) with X being a leaving group like halogen or —S(O)Me in a nucleophilic aromatic substitution (S_{N}Ar) or palladium catalyzed Buchwald-Hartwig amination. The procedures that can be applied are in analogy to those described for the synthesis of (A3) from (A1) and (A2a) in Scheme 1.

Carboxylic acid derivatives of general formula (A21) may be synthesized according to the route depicted in Scheme 3. Nitro compounds of general formula (A15) can be obtained from reacting monoprotected diamines (A1) or their corresponding salts and nitroaryls (A14) with X being a leaving group like chlorine in a nucleophilic aromatic substitution (S_{N}Ar). The reaction can be carried out in the presence of inorganic bases like K₂CO₃ in polar solvents such as for example DMSO at temperatures between room temperature and the boiling point of the solvent for 2-12 h. Nitroaryls of general formula (A14) are either commercially available or can be prepared according to procedures available from the public domain.

Anilines of general formula (A16) may be obtained from nitro compounds of general formula (A15) by reduction. For reduction all methods that are known in the art may be applied. For example, nitro compounds of general formula (A15) can be reacted in the presence of a metal catalyst like palladium on charcoal under an atmosphere of hydrogen gas (1-5 bar) in polar, protic solvents like methanol or ethanol at temperatures between 0° C. and the boiling point of the solvent for 15-24 h.

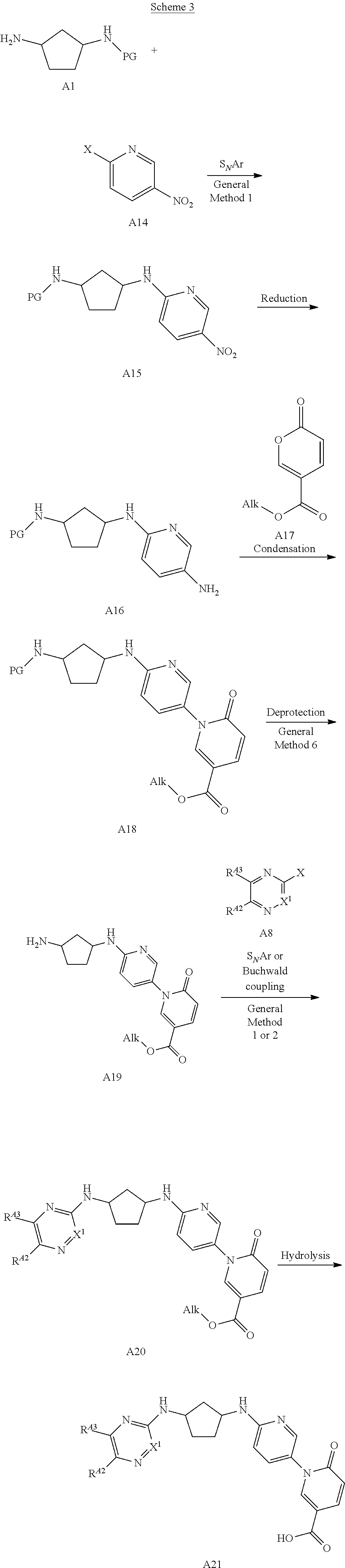

Scheme 3: Routes for the preparation of compounds of general formula (A21) in which X is a leaving group, PG is a protective group, Alk is methyl or ethyl and X¹, R^{A2} and R^{A3} have the meaning as given for general formula (I-B), supra.

Pyridones of general formula (A18) may be obtained from anilines (A16) by condensation with oxo-pyranes of general formula (A17) with Alk being methyl or ethyl in polar, protic solvents like ethanol at temperatures between room temperature and the boiling point of the solvent for 2-12 h. Oxo-pyranes of general formula (A17) are either commercially available or can be prepared according to procedures available from the public domain.

Primary amines of general formula (A19) may be synthesized from (A18) via removal of the protective group. For deprotection the same procedures apply as described for the synthesis of (A7) from (A6) in Scheme 1.

Carboxylic esters of general formula (A20) can be obtained from primary amines (A19) and heteroaryls (A8) via nucleophilic aromatic substitution (S_{N}Ar) or palladium catalyzed Buchwald-Hartwig amination applying procedures in analogy to those described for the synthesis of (A3) from (A1) and (A2a) in Scheme 1.

Carboxylic acid derivatives of general formula (A21) may be synthesized from carboxylic esters of general formula (A20) by ester hydrolysis. For saponification all methods that are known in the art may be applied. For example, esters (A20) may be reacted with bases like sodium hydroxide in polar, protic solvents like methanol, water or mixtures thereof at temperatures between 0° C. and the boiling point of the solvent for 0.5-2 h.

For the synthesis of final compounds of general formula (B-2) routes and methods comparable to the ones described in Schemes 1-3 may be applied. Without being intended to be limiting the routes to compounds of general formula (A43) in Schemes 6-7 are given for further illustration. Diamines of general formula (A39) are either commercially available or can be prepared according to procedures available from the public domain.

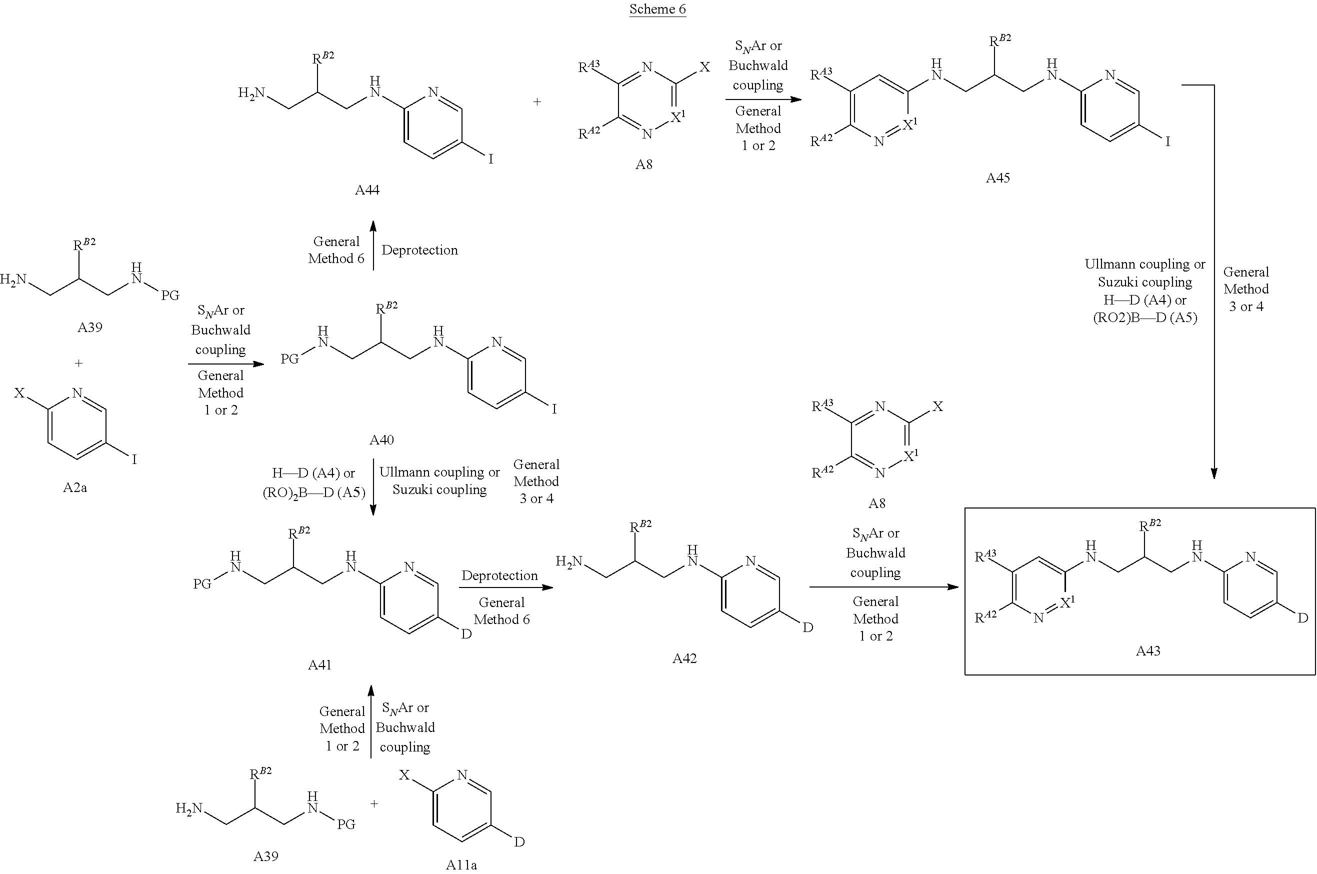

Scheme 6: Routes for the preparation of compounds of general formula (A43) in which X is a leaving group, PG is a protective group and D, X¹, R^{A2}, R^{A3} and R^{B2} have the meaning as given for general formulae (I-B) and (B-2), supra.

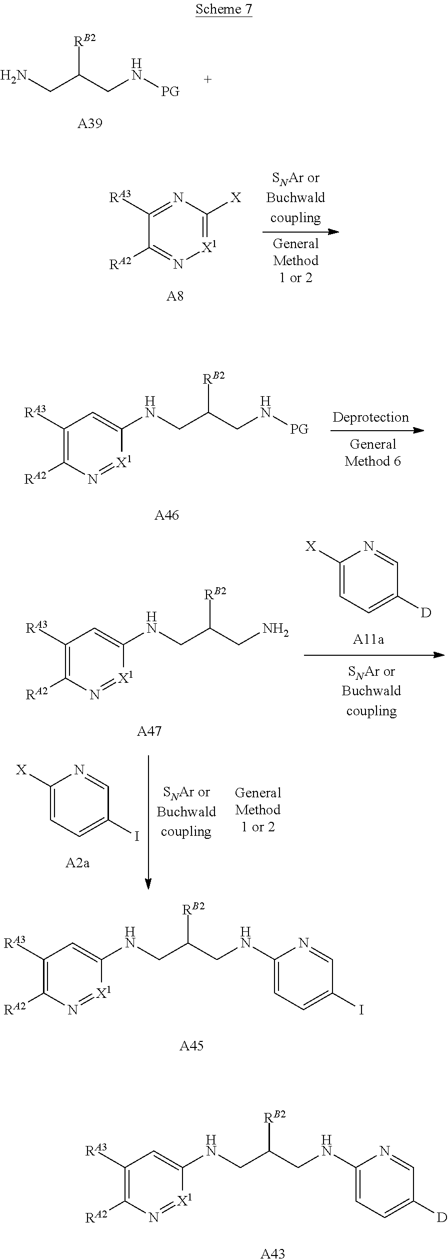

Scheme 7: Routes for the preparation of compounds of general formula (A43) and intermediates (A45) in which X is a leaving group, PG is a protective group and D, X¹, R^{A2}, R^{A3} and R^{B2} have the meaning as given for general formulae (I-B) and (B-2), supra.

Further compounds with different formulae as described above can be prepared by similar methods.

### EXPERIMENTAL SECTION

NMR peak forms are stated as they appear in the spectra, possible higher order effects have not been considered.

The following table lists the abbreviations used in this paragraph and in the examples section as far as they are not explained within the text body. Other abbreviations have their meanings customary per se to the skilled person.

### Abbreviations

Aq Aqueous
BINAP 2,2′-Bis(diphenylphosphaneyl)-1,1′-binaphthalene
Boc tert-Butoxycarbonyl
Brine Saturated aqueous sodium chloride solution
nBuOH Butan-1-ol
CataCXium A Di(adamantan-1-yl)(butyl)phosphane (CAS Registry Number 321921-71-5)
CataCXium A Pd G3 Methanesulfonato(diadamantyl-n-butylphosphino)-2′-amino-1,1′ biphenyl-2-yl)palladium(II) precatalyst generation 3
CDI 1,1′-Carbonyldiimidazole
CHAPS 3-[(3-Cholamidopropyl)dimethylammonio]-1-propanesulfonate
m-CPBA 3-Chlorobenzoperoxoic acid
DavePhos 2′-(Dicyclohexylphosphaneyl)-N¹,N²-dimethyl-[1,1′-biphenyl]-2-amine (CAS Registry Number 213697-53-1)
dba (all-trans)-Dibenzylideneacetone
DCC Dicyclohexylcarbodiimide
DCM Dichloromethane
DEA Diethylamine
DIPEA N-ethyl-N-isopropyl-propan-2-amine
DMA N,N-Dimethylacetamide
DME Dimethoxyethane
DMF N,N-Dimethylformamide
DMSO Dimethyl sulfoxide
DPPA Diphenyl phosphorazidate
dppf 1,1′-Bis(diphenylphosphino)ferrocene
EDC 2-(((Ethylimino)methylene)amino)-N¹,N²-dimethylethan-1-amine hydrochloride
ESI Electrospray ionization
EtOAc Ethyl acetate
EtOH Ethanol
FA Formic acid
G3 Generation 3
GMean Geometric mean
HATU (1-(Bis(dimethylamino)methylene]-1H-1,2,3-triazolo[4,5-b]pyridinium-3-oxo hexafluorophosphate
HEPES (4-(2-Hydroxyethyl)-1-piperazineethanesulfonic acid)
HOAt 3H-[1,2,3]Triazolo[4,5-b]pyridin-3-ol
HOBt 1H-Benzo[d][1,2,3]triazol-1-ol
HPLC High performance liquid chromatography
HRMS High resolution mass spectrometry
IC₅₀ Half maximal inhibitory concentration
K_{D} Dissociation constant
MeCN Acetonitrile
MeOH Methanol
MS Mass spectrometry
MTBD 7-Methyl-1,5,7-triazabicyclo[4.4.0]dec-5-ene
MTBE Methyl tert-butyl ether
MW Microwave
NBS N-Bromosuccinimide
NMP 1-Methylpyrrolidin-2-one
NMR Nuclear magnetic resonance
O.n Overnight
OTf Trifluoromethanesulfonate
Pd-118/PdCl₂(dtbpf) [1,1′-Bis(di-tert-butylphosphino)ferrocene]dichloropalladium(II)
Pd₂(dba)₃ Tris(dibenzylideneacetone)dipalladium (0)
Pd₂(dba)₃-CHCl₃ Tris(dibenzylideneacetone)dipalladium(0) chloroform adduct
PdCl₂(dppf) [1,1′-Bis(diphenylphosphino)ferrocene]dichloropalladiun(II)
Pd-PEPPSI-IpentCl
2-methylpyridine (SP-4-1)-[1,3-Bis[2,6-bis(1-ethylpropyl)phenyl]-4,5-dichloro-1,3-dihydro-2H-imidazol-2-ylidene]dichloro(2-methylpyridine)palladium (CAS Registry Number 1612891-29-8)
PE Petroleum ether
PG Protective group
prep. Preparative
PyBOP ((1H-Benzo[d][1,2,3]triazol-1-yl)oxy)tri(pyrrolidin-1-yl)phosphonium hexafluorophosphate
qToF Quadrupole time-of-flight
RockPhos Di-tert-butyl(2′,4′,6′-triisopropyl-3-methoxy-6-methyl-[1,1′-biphenyl]-2-yl)phosphine
rt Room temperature
sat. Saturated
SFC Supercritical fluid chromatography
S_{N}Ar Nucleophilic aromatic substitution
T3P 2,4,6-Tripropyl-1,3,5,2,4,6-trioxatriphosphinane 2,4,6-trioxide
TEA Triethylamine
TFA Trifluoroacetic acid
TFAA 2,2,2-Trifluoroacetic anhydride
THE Tetrahydrofurane
TLC Thin layer chromatography
TMEDA N¹,N¹,N²,N²-Tetramethylethane-1,2-diamine
t_{R} Retention time
UPLC Ultra high performance liquid chromatography
XantPhos 4,5-Bis(diphenylphosphino)-9,9-dimethylxanthene, (9,9-dimethyl-9H-xanthene-4,5-diyl)bis(diphenylphosphine)
XPhos Dicyclohexyl(2′,4′,6′-triisopropyl-[1,1′-biphenyl]-2-yl)phosphane

Units
atm atmosphere
C Celcius
g gram
h hour(s)
Hz hertz
L liter
M mole per liter
mg milligram
MHz megahertz
min minute(s)
mL milliliter
mm millimeter
mM millimole per liter
mol mole
mmol millimole(s)
nA nanoampere
nL nanolitre
μm micrometer
μL microliter
N equivalents per liter
nm nanometer
ppm parts per million
Å Ångström

The various embodiments described in this application are illustrated by the following examples which are not meant to limit the compound of Formula (I) in any way.

The example testing experiments described herein serve to illustrate the present embodiments and is not limited to the examples given.

### EXPERIMENTAL SECTION—GENERAL PART

### General Conditions

(i) operations were carried out at room temperature (rt), i.e. in the range 17 to 28° C. and were needed under an atmosphere of an inert gas such as N₂;
(ii) where reactions refer to being degassed or purged, this can be performed for example by purging the reaction solvent with a constant flow of nitrogen for a suitable period of time (for example 5 to 10 min) or by repeatedly evacuating the vessel and backfill with appropriate inert atmosphere (for example nitrogen (g) or argon (g));
(iii) where reactions refer to the use of a microwave reactor, one of the following microwave reactors were used: Biotage Initiator, Personal Chemistry Emrys Optimizer, Personal Chemistry Smith Creator or CEM Explorer;
(iv) in general, the course of reactions was followed by thin layer chromatography (TLC) and/or analytical high performance liquid chromatography (HPLC or UPLC) which was usually coupled to a mass spectrometer (LCMS);
(v) to remove excess water, organic solutions were dried over anhydrous MgSO₄ or Na₂SO₄, or by using ISOLUTE® Phase Separator, and work-up procedures were carried out using traditional phase separating techniques;
(vi) evaporations were carried out either by rotary evaporation in vacuo or in a Genevac™ HT-4/EZ-2 or Biotage® V10;
(vii) unless otherwise stated, flash column chromatography was performed on normal phase silica, using either Merck Silica Gel (Art. 9385) or pre-packed cartridges such as Biotage® SNAP cartridges (40-63 μm silica, 4-330 g), Biotage® Sfar Silica HC D cartridges (20 μm, 10-100 g), Interchim puriFlash™ cartridges (25 μm, 4-120 g), Interchim puriFlash™ cartridges (50 μm, 25-330 g), Grace™ GraceResolv™ Silica Flash Cartridges (4-120 g) or Agela Flash Colum Silica-CS cartridges (80-330 g), or on reverse phase silica using Agela Technologies C-18, spherical cartridges (20-35 μm, 100 A, 80-330 g), manually or automated using a Grace Reveleris® X2 Flash system or similar system;
(viii) preparative reversed phase HPLC and preparative reversed phase SFC were performed using standard HPLC and SFC instruments, respectively, equipped with either a MS and/or UV triggered fraction collecting instrument, using either isocratic or a gradient of the mobile phase as described in the experimental section;
relevant fractions were collected, combined and freeze-dried or evaporated to give the purified compound or relevant fractions were collected, combined and concentrated at reduced pressure, extracted with DCM or EtOAc, and the organic phase was dried either over Na₂SO₄ or by using a phase-separator, and then concentrated at reduced pressure to give the purified compound;
(ix) chiral preparative chromatography was carried out using HPLC or SFC on a standard HPLC or SFC instrument, respectively, and using either isocratic or gradient run with mobile phase as described in the experimental section;
(x) preparative thin layer chromatography (TLC) was performed using TLC glass plates and applying a suitable solvent or mixture of solvents;
(xi) yields, where present, are not necessarily the maximum attainable, and some, reactions were repeated if a larger amount of the reaction product was required;
(xii) where certain compounds were obtained as an acid-addition salt, for example a mono-hydrochloride salt or a di-hydrochloride salt, the stoichiometry of the salt was based on the number and nature of the basic groups in the compound, the exact stoichiometry of the salt was generally not determined, for example by means of elemental analysis data. Where stated the salts were treated according to literature-known processes to generate the corresponding free base prior to being used;
(xiii) in general, the structures of the end-products of the Formula (I) were confirmed by nuclear magnetic resonance (NMR) and/or mass spectral techniques; proton NMR chemical shift values were measured on the delta scale using Bruker Avance III 300, 400, 500 and 600 spectrometers, operating at ¹H frequencies of 300, 400, 500 and 600 MHz, respectively. The experiments were typically recorded at 25° C. Chemical shifts are given in ppm with the solvent as internal standard. Protons on heteroatoms such as NH and OH protons are only reported when detected in NMR and can therefore be missing. In certain instances, protons can be masked or partially masked by solvent peaks and will therefore either be missing and not reported or reported as multiplets overlapping with solvent. The following abbreviations have been used (and derivatives thereof, e.g. dd, doublet of doublets, etc.): s, singlet; d, doublet; t, triplet; q, quartet; m, multiplet; br, broad; qn, quintet; p, pentet. Electrospray mass spectral data were obtained using a Waters Acquity UPLC coupled to a Waters single quadrupole mass spectrometer or similar equipment, acquiring both positive and negative ion data, and generally, only ions relating to the parent structure are reported; high resolution electrospray mass spectral data were obtained using a Waters XEVO qToF mass spectrometer or similar equipment, coupled to a Waters Acquity UPLC, acquiring either positive and negative ion data, and generally, only ions relating to the parent structure are reported;
(xiv) intermediates were in certain cases not fully purified but their structures and purity were assessed by TLC, analytical HPLC/UPLC, and/or NMR analysis and/or mass spectrometry;
(xv) certain intermediates were isolated as TFA salts and can contain excess TFA. The excess can be calculated from the weight of the crude sample. Where stated the salts were treated according to literature-known processes to generate the corresponding free base prior to being used;
(xvi) unless stated otherwise compounds containing an asymmetric carbon and/or sulfur atom were not resolved;
(xvii) in general Examples and Intermediate compounds are named using ChemDraw Professional version 21.0.0.28 from PerkinElmer. ChemDraw Professional version 21.0.0.28 generates the names of chemical structures using the Cahn-Ingold-Prelog (CIP) rules for stereochemistry and follows IUPAC rules as closely as possible when generating chemical names. Stereoisomers are differentiated from each other by stereodescriptors cited in names and assigned in accordance with the CIP rules.

Where applicable ChemDraw is using labels in the graphical representation of stereocenters such as ‘&’ and ‘or’ to describe the configuration of the stereochemical centers present in the structure. A number following the ‘&’ and ‘or’ flag is assigned to each stereocenter present.

In some cases, the purification methods as described above can provide those compounds of Formula (I) which possess a sufficiently basic or acidic functionality in the form of a salt, such as, in the case of a compound of Formula (I) which is sufficiently basic, a trifluoroacetate or formate salt for example, or, in the case of a compound of Formula (I) which is sufficiently acidic, an ammonium salt for example. A salt of this type can either be transformed into its free base or free acid form, respectively, by various methods known in the art or be used as salt in subsequent biological assays. It is to be understood the specific form (e.g. salt, free base etc.) of a compound of Formula (I) as isolated and as described herein is not necessarily the only form in which said compound can be applied to a biological assay in order to quantify the specific biological activity.

### Purification Methods

### Preparative HPLC Methods:

PrepMethod A: The compound was purified by preparative HPLC on a XBridge™ C18 OBD column (5 μm, 150×30 mm ID) using a gradient of MeOH in a H₂O/NH₄HCO₃ (10 mM)/NH₃ (0.1%, aq) buffer system as mobile phase;

PrepMethod B: The compound was purified by preparative HPLC on a YMC-Actus Triart C18 ExRS column (5 μm, 150×30 mm ID) using a gradient of MeCN in H₂O/NH₄HCO₃ (10 mM)/NH₃ (0.1%, aq) buffer system as mobile phase;

PrepMethod C: The compound was purified by preparative HPLC on a XBridge™ Shield RP18 OBD column (5 μm, 150×30 mm ID) using a gradient of MeCN in H₂O/NH₄HCO₃ (10 mM)/NH₃ (0.1%, aq) buffer system as mobile phase;

PrepMethod D: The compound was purified by preparative HPLC on a XBridge™ C18 OBD column (5 μm, 150×30 mm ID) using a gradient of MeCN in a H₂O/NH₄HCO₃ (10 mM)/NH₃ (0.1%, aq) buffer system as mobile phase;

PrepMethod E: The compound was purified by preparative HPLC on a Waters Xselect CSH C18 OBD column (5 μm, 150×30 mm ID) using a gradient of MeCN in H₂O/FA (0.1%) buffer system as mobile phase;

PrepMethod F: The compound was purified by preparative HPLC on a XBridge™ C18 column (10 μm, 250×50 mm ID) using a gradient of MeCN in H₂O/MeCN/NH₃ (95/5/0.2) buffer system as mobile phase;

PrepMethod G: The compound was purified by preparative HPLC on a Kromasil C18 column (10 μm, 250×20 mm ID) using a gradient of MeCN in H₂O/MeCN/FA (95/5/0.2) buffer system as mobile phase;

PrepMethod H: The compound was purified by preparative HPLC on a XBridge™ C18 column (10 μm, 250×19 mm ID) using a gradient of MeCN in H₂O/MeCN/NH₃ (95/5/0.2) buffer system as mobile phase;

PrepMethod I: The compound was purified by preparative HPLC on a XBridge™ C18 ODB column (5 μm, 150×19 mm ID) using a gradient of MeCN in H₂O/NH₄HCO₃ (10 mM, pH 9) buffer system as mobile phase.

PrepMethod J: The compound was purified by preparative HPLC on a Waters™ Sunfire™ C18 OBD column (5 μm, 150×30 mm ID) using a gradient of MeCN in H₂O/FA (0.1%) as mobile phase;

PrepMethod K: The compound was purified by preparative HPLC on a YMC-Actus Triart C18 column (5 μm, 150×30 mm ID) using a gradient of MeCN in H₂O/NH₄HCO₃ (10 mM)/NH₃ (0.05%, aq) buffer system as mobile phase;

PrepMethod L: The compound was purified by preparative HPLC on a Waters™ Sunfire™ C18 OBD column (5 μm, 150×30 mm ID) using a gradient of MeCN in a H₂O/NH₄HCO₃ (10 mM)/NH₃ (0.1%, aq) buffer system as mobile phase;

PrepMethod M: The compound was purified by preparative HPLC on a YMC-Actus Triart C18 column (5 μm, 150×30 mm ID) using a gradient of MeCN in H₂O/NH₄HCO₃ (10 mM)/NH₃ (0.1%, aq) buffer system as mobile phase;

PrepMethod N: The compound was purified by preparative HPLC on a XBridge™ OBD Phenyl column (5 μm, 150×19 mm ID) using a gradient of MeCN in a H₂O/NH₄HCO₃ (10 mM)/NH₃ (0.1%, aq) buffer system as mobile phase;

PrepMethod 0: The compound was purified by preparative HPLC on a XBridge™ C18 OBD column (5 μm, 150×30 mm ID) using a gradient of MeCN in a H₂O/NH₄HCO₃ (10 mM)/NH₃ (0.05%, aq) buffer system as mobile phase;

PrepMethod P: The compound was purified by preparative HPLC on a Waters Xselect CSH C18 OBD column (5 μm, 150×30 mm ID) using a gradient of MeCN in H₂O as mobile phase;

PrepMethod Q: The compound was purified by preparative HPLC on a XBridge™ Shield RP18 OBD column (5 μm, 250×19 mm ID) using a gradient of MeCN in H₂O/NH₄HCO₃ (10 mM)/NH₃ (0.05%, aq) buffer system as mobile phase;

PrepMethod R: The compound was purified by preparative HPLC on a Waters™ Sunfire™ C18 OBD column (5 μm, 150×30 mm ID) using a gradient of MeCN in H₂O/FA (10 mM) as mobile phase;

PrepMethod S: The compound was purified by preparative HPLC on a XBridge™ C18 OBD column (5 μm, 150×30 mm ID) using a gradient of MeCN in a H₂O/NH₄HCO₃ (10 mM) buffer system as mobile phase;

PrepMethod T: The compound was purified by preparative HPLC on a XBridge™ C18 OBD column (5 μm, 150×30 mm ID) using a gradient of 20 mM NaOH+10% MeCN in a H₂O/NH₄HCO₃ (10 mM)/NH₃ (0.05%, aq) buffer system as mobile phase;

PrepMethod U: The compound was purified by preparative HPLC on a XBridge™ OBD Phenyl column (5 μm, 250×19 mm ID) using a gradient of MeCN in a H₂O/NH₄HCO₃ (10 mM)/NH₃ (0.05%, aq) buffer system as mobile phase;

PrepMethod V: The compound was purified by preparative HPLC on a XBridge™ OBD Phenyl column (5 μm, 250×19 mm ID) using a gradient of MeCN in a H₂O/TFA (0.05%) buffer system as mobile phase;

PrepMethod X: The compound was purified by preparative HPLC on a YMC-Actus Triart C18 ExRS column (5 μm, 150×30 mm ID) using a gradient of MeCN in H₂O/NH₄HCO₃ (10 mM)/NH₃ (0.05%, aq) buffer system as mobile phase;

PrepMethod Y: The compound was purified by preparative HPLC on a XBridge™ Shield RP18 OBD column (5 μm, 100×30 mm ID) using a gradient of MeCN in H₂O/NH₄HCO₃ (10 mM)/NH₃ (0.05%, aq) buffer system as mobile phase;

PrepMethod Z: The compound was purified by preparative HPLC on a Waters Xselect Peptide CSH C18 OBD column (5 μm, 150×30 mm ID) using a gradient of MeCN in H₂O/FA (0.1%) buffer system as mobile phase;

PrepMethod Z1: The compound was purified by preparative HPLC on a Waters Xselect Peptide CSH C18 OBD column (5 μm, 150×30 mm ID) using a gradient of MeCN in a H₂O/NHaHCO₃ (10 mM)/NH₃ (0.1%, aq) buffer system as mobile phase;

PrepMethod Z2: The compound was purified by preparative HPLC on a Waters Xselect CSH C18 OBD column (5 μm, 150×30 mm ID) using a gradient of MeCN in a H₂O/NH₄HCO₃ (10 mM)/NH₃ (0.05%, aq) buffer system as mobile phase;

PrepMethod Z3: The compound was purified by preparative HPLC on a XBridge™ C18 OBD column (5 μm, 100×30 mm ID) using a gradient of MeCN in a H₂O/NH₄HCO₃ (10 mM)/NH₃ (0.05%, aq) buffer system as mobile phase;

PrepMethod Z4: The compound was purified by preparative HPLC on a XBridge™ C18 OBD column (5 μm, 250×19 mm ID) using a gradient of MeCN in a H₂O/TFA (0.005%) buffer system as mobile phase;

PrepMethod Z5: The compound was purified by preparative HPLC on a Waters Xselect CSH C18 OBD column (5 μm, 250×19 mm ID) using a gradient of MeOH in a H₂O/NH₄HCO₃ (10 mM)/NH₃ (0.05%, aq) buffer system as mobile phase;

PrepMethod Z6: The compound was purified by preparative HPLC on a XBridge™ Shield RP18 OBD column (5 μm, 100×30 mm ID) using a gradient of MeCN in H₂O/NH₄HCO₃ (10 mM)/NH₃ (0.1%, aq) buffer system as mobile phase;

PrepMethod Z7: The compound was purified by preparative HPLC on a XBridge™ C18 OBD column (5 μm, 100×50 mm ID) using a gradient of MeCN in a H₂O/NH₄HCO₃ (10 mM)/NH₃ (0.1%, aq) buffer system as mobile phase;

PrepMethod Z8: The compound was purified by preparative HPLC on a XBridge™ RP18 OBD column (5 μm, 100×30 mm ID) using a gradient of MeCN in H₂O/NH₄HCO₃ (10 mM)/NH₃ (0.1%, aq) buffer system as mobile phase.

PrepMethod Z9: The compound was purified by preparative HPLC on a XBridge™ C18 column (5 μm, 250×20 mm ID) using a gradient of MeCN in H₂O/MeCN/NH₃ (95/5/0.2) buffer system as mobile phase;

PrepMethod Z10: The compound was purified by preparative HPLC on a XBridge™ C18 column (5 μm, 250×50 mm ID) using a gradient of MeCN in H₂O/MeCN/NH₃ (95/5/0.2) buffer system as mobile phase.

### Preparative SFC Methods:

PrepMethod SFC-A: The compound was purified by preparative SFC on a Waters™ BEH (5 μm, 250×30 mm ID) using MeOH/H₂O (NH₃, 50 mM) (97/3) in CO₂ as mobile phase;

PrepMethod SFC-B: The compound was purified by preparative SFC on a Phenomenex Luna Hilic (3.5 μm, 100×3 mm ID) using MeOH/NH₃ 20 mM in CO₂ as mobile phase;

PrepMethod SFC-C: The compound was purified by preparative SFC on a Waters™ Acquity UPC2 BEH (3.5 μm, 100×3 mm ID) using MeOH/H₂O (NH₃, 50 mM) (97/3) in CO₂ as mobile phase.

PrepMethod SFC-D: The compound was purified by preparative SFC on a Waters™ BEH (5 μm, 250×30 mm ID) using MeOH/NH₃ (20 mM) in CO₂ as mobile phase;

PrepMethod SFC-E: The compound was purified by preparative SFC on a Phenomenex Luna Hilic (5 μm, 250×30 mm ID) using MeOH/NH₃ 20 mM in CO₂ as mobile phase.

### Preparative HPLC Methods for Parallel Experiment Set-Up:

PrepMethod Parallel A: The compound was purified by preparative HPLC on a Waters™ XBridge™ C₁₈ column (5 μm, 100×10 mm ID) using a gradient (2-94%) of MeCN in H₂O/NH₃ (pH 10) buffer system as mobile phase;

PrepMethod Parallel B: The compound was purified by preparative HPLC on a Waters™ Xselect™ CSH Fluoro Phenyl column (5 μm, 100×10 mm ID) using a gradient (2-94%) of MeCN in H₂O/FA (pH 3) buffer system as mobile phase.

PrepMethod Parallel C: The compound was purified by preparative HPLC on a Waters™ XBridge™ C18 OBD column (5 μm, 150×19 mm ID) using a gradient (5-95%) of MeCN in H₂O/MeCN/NH₃ (95/5/0.2) (pH 10) buffer system as mobile phase;

PrepMethod Parallel D: The compound was purified by preparative HPLC on a XBridge™ 018 OBD column (5 μm, 150×19 mm ID) using a gradient (5-95%) of MeCN in a H₂O/NH₄HCO₃ (10 mM) (pH 9) buffer system as mobile phase.

### Synthesis Methods

### General Method 1 (GM1): Nucleophilic Aromatic Substitution (S_{N}Ar)

### Condition a (GM1A): Conventional Heating

To a solution of the respective amine nucleophile or its salt (1 eq.) in DMSO (alternatively NMP or n-BuOH or 1,4-dioxane) is added base (1-7 eq.) and the respective heteroaryl electrophile (0.9-2 eq.) at rt and the resulting mixture stirred under heating (100-130° C.) until TLC and/or LCMS indicate complete consumption of the starting material (typically overnight). The reaction mixture is either concentrated under reduced pressure and the obtained crude material subjected to chromatography or prep. TLC and/or prep. HPLC to give the desired aniline product.

In an alternative aqueous work-up, the reaction mixture is poured into sat. brine or water and extracted with EtOAc. The combined organic layers are washed with water or brine, dried over Na₂SO₄, filtered and evaporated and the obtained crude material subjected to chromatography on silica to give the desired aniline product.

### Condition B (GM1B): Microwave Heating

To a mixture of the respective amine nucleophile or its salt (1 eq.) and the respective heteroaryl electrophile (1-1.5 eq.) in a microwave vial is added NMP and base (1-3 eq.), the vial capped and heated under microwave radiation until TLC and/or LCMS indicate consumption of the starting material (typically 0.5-2 h). The reaction mixture is concentrated under reduced pressure and the obtained crude material subjected to prep. HPLC to give the desired aniline product.

### General Method 2 (GM2): Buchwald-Hartwig Amination

A solution of the respective amine nucleophile or its salt (1 eq.) in 1,4-dioxane under nitrogen at rt is treated with the respective heteroaryl electrophile (0.5-4 eq.), Cs₂CO₃ (1.2-5 eq.) and Pd PEPPSI-IpentCl [CAS 1612891-29-8] (3-7 mol %) and the reaction mixture stirred under heating (100° C.) until TLC and/or LCMS indicate complete consumption of the starting material (typically overnight). The reaction mixture is either filtered through a pad of Celite or silica, the filter cake washed with EtOAc and the combined filtrates concentrated under reduced pressure. The obtained crude material is either triturated with PE/EtOAc or subjected to prep. TLC and/or prep. HPLC to give the desired aniline product.

In an alternative aqueous work-up, the reaction mixture is concentrated under reduced pressure and the residue partitioned between EtOAc and water. The phases are separated and the aqueous phase is extracted with EtOAc. The combined organic layers are washed with water and/or brine, dried over Na₂SO₄, filtered and evaporated and the obtained crude material subjected to prep. TLC and/or prep. HPLC to give the desired aniline product.

### General Method 3 (GM3): Ullmann Coupling

A solution of the respective aryl halide (1 eq.) in 1,4-dioxane under nitrogen at rt is treated with the respective heteroatom nucleophile H-D (A4) (1-10 eq.), Cs₂CO₃ (3-6 eq.), Cu(I)I (0.2-2 eq.) and rel-(1R,2R)—N¹,N²-dimethylcyclohexane-1,2-diamine [CAS 67579-81-1] (0.2-2 eq.) and the reaction mixture stirred under heating (80-100° C.) until TLC and/or LCMS indicate complete consumption of the starting material (typically 15-18). The reaction mixture is diluted with EtOAc and washed sequentially with water and brine. Alternatively, the reaction mixture is diluted with water or brine and the aqueous layer extracted with EtOAc. The organic layer is dried over Na₂SO₄, filtered and evaporated. The obtained crude material is either triturated with PE/EtOAc or subjected to prep. TLC and/or prep. HPLC to give the desired coupling product.

In an alternative non-aqueous work-up, the reaction mixture is filtered through a pad of Celite, the filter cake washed with DCM and the combined filtrates concentrated under reduced pressure. The obtained crude material is subjected to prep. TLC and/or prep. HPLC to give the desired coupling product.

### Condition a (GM3A): Parallel Experiment Set-Up

To a mixture of the respective heteroatom nucleophile H-D (A4) (50 μmol, 2 eq.) and a pre-mix of Cu(I)I (1.9 mg, 10 μmol, 0.40 eq.) and K₂CO₃ (10.4 mg, 75.0 μmol, 3.0 equiv.) under argon was added a stock solution of (1S,3S)—N¹-(5-(difluoromethoxy)pyrazin-2-yl)-N3-(5-iodopyridin-2-yl)cyclopentane-1,3-diamine (Intermediate 19) compound i-19d (11 mg, 25 μmol, 1.0 eq.) and rel-(1R,2R)—N¹,N²-dimethylcyclohexane-1,2-diamine [CAS 67579-81-1] (2.8 mg, 20 μmol, 0.80 eq.) in DMSO (100 μL). The reaction mixtures were stirred at 120° C. for 17-22 h and then diluted with DMSO (500 μL). SiliaMetS™ Imidazole Metal Scavenger (42 mg, 50 μmol, loading: 1.20 mmol/mg) in DMSO (200 μL) was added to each vial and the mixtures were shaken on a Bioshake® microplate shaker at 800 rpm at 25° C. overnight. The reaction mixtures were transferred to a 24-well filter plate and filtered to 4 mL high-recovery vials using a Biotage® Pressure+manifold. The vials were rinsed with DMSO (1 mL) and the solutions were passed through the filter plate. The filtered solutions were concentrated in a Genevac™ centrifugal concentrator, re-constituted in DMSO (300 μL) and purified by preparative HPLC to give the desired coupling product.

### General Method 4 (GM4): Suzuki Coupling

Condition a (GM4A): Coupling with Boronic Acid Derivatives

A solution of the respective aryl halide (1 eq.) in a mixture of 1,4-dioxane and water under nitrogen at rt is treated with the respective boronic acid derivative (A5) (1.5-2 eq.) or (A26) (10 eq.), Cs₂CO₃ (3 eq.) or K₂CO₃ (3 eq.) or K₃PO₄ (2-3 eq.) and 1,1′-bis(di-tert-butylphosphino)ferrocene palladium dichloride [CAS 95408-45-0] (5-10 mol %) or 1,1′-bis(diphenylphosphino)ferrocene palladium dichloride [CAS 72287-26-4] (10 mol %) and the reaction mixture stirred under heating (80-100° C.) until TLC and/or LCMS indicate complete consumption of the starting material (2-18). The reaction mixture is filtered through a pad of Celite, the filter cake washed with DCM and the combined filtrates concentrated under reduced pressure. The obtained crude material is subjected to prep. TLC and/or prep. HPLC to give the desired coupling product.

In an alternative aqueous work-up, the reaction mixture is diluted with water, the phases separated and the aqueous phase extracted with EtOAc. The combined organic layers are dried over Na₂SO₄, filtered and evaporated and the obtained crude material subjected to prep. TLC and/or C18-flash chromatography or prep. HPLC to give the desired coupling product.

Condition B (GM48): Coupling with Trifluoroborates

A solution of the respective aryl halide (1 eq.) in 1,4-dioxane under nitrogen at rt is treated with the respective potassium trifluoroborate (A25) (4 eq.), Cs₂CO₃ (4 eq.) and CataCXium A Pd G3 [CAS 1651823-59-4] (20 mol %) and the reaction mixture stirred under heating (100° C.) until TLC and/or LCMS indicate complete consumption of the starting material (typically 15). The reaction mixture is filtered through a pad of Celite, the filter cake washed with DCM and the combined filtrates concentrated under reduced pressure. The obtained crude material is subjected to prep. TLC and/or prep. HPLC to give the desired coupling product.

### General Method 5 (GM5): Chan-Lam Coupling

A mixture of the respective heteroatom nucleophile H-D (A4) (1 eq.) in 1,4-dioxane at rt is treated with the respective boronic acid (2-3 eq.), TMEDA (3 eq.) and Cu(OTf)₂ (1.2-2 eq.) and the reaction mixture stirred under heating (100° C.) until TLC and/or LCMS indicate complete consumption of the starting material (typically 16). The reaction mixture is filtered through a pad of Celite, the filtrate concentrated under reduced pressure and the residue partitioned between EtOAc and water. The aqueous layer is extracted with EtOAc, the combined organic layers washed with water, dried over Na₂SO₄, filtered and evaporated. The obtained crude material is subjected to C18-flash chromatography to give the desired coupling product.

### General Method 6 (GM6): Boc Deprotection

Condition A (GM6A): Deprotection with HCl

A solution of the respective Boc protected amine (1 eq.) in MeOH at rt is treated with 4 M HCl in MeOH (13-77 eq.) and the reaction mixture stirred under heating (60-80° C.) until TLC and/or LCMS indicate complete consumption of the starting material (typically 2-3). The reaction mixture is concentrated under reduced pressure to give the desired amine as (an unspecified) HCl salt.

Condition B (GM6B): Deprotection with TFA

A solution of the respective Boc protected amine (1 eq.) in DCM at rt is treated with TFA (11-190 eq.) and the reaction mixture stirred at rt until TLC and/or LCMS indicate complete consumption of the starting material (3-16). The reaction mixture is concentrated under reduced pressure to give the desired amine as (an unspecified) TFA salt.

### General Method 7 (GM7): Amide Formation

In a microwave vial a solution of the respective ester (A35) in MeOH is treated with the respective amine or its salt (A36) (4-100 eq., neat or as a solution in THF) and where applicable additionally with DIPEA (4-8 eq.) at rt. The vial is capped, and the reaction mixture stirred at rt or heating (60-70° C.) until TLC and/or LCMS indicate complete consumption of the starting material (3). The reaction mixture is concentrated under reduced pressure and the obtained crude material subjected to preparative HPLC to give the desired amide.

### General Method 8 (GM8): Methyl-thioether Oxidation

A solution of the methyl-thioether (1 eq.) in DCM is cooled to 0° C. and treated slowly with 3-chlorobenzoperoxoic acid (m-CPBA) [CAS 937-14-4] (0.9-1.2 eq.) and the resulting mixture stirred at rt until TLC and/or LCMS indicate complete consumption of the starting material (0.5-2). The reaction mixture is directly used in the next step.

Alternatively, the reaction mixture is concentrated under reduced pressure and the obtained crude material is subjected to flash chromatography on silica to give the desired oxidation product.

### INTERMEDIATES

### Intermediate 1

### 6′-(((1S,3S)-3-Aminocyclopentyl)amino)-2H-[1,3′-bipyridin]-2-one

### Step A. i-1a

### tert-Butyl ((1S,3S)-3-((5-iodopyridin-2-yl)amino)cyclopentyl)carbamate

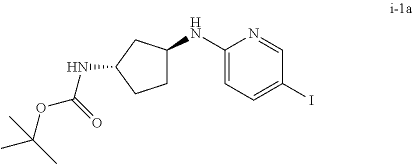 [SMILES: CC(C)(C)OC(=O)N[C@H]1CC[C@H](Nc2ccc(I)cn2)C1]

According to GM1A 2-fluoro-5-iodopyridine (CAS Reg. No. 171197-80-1) (2.23 g, 9.99 mmol) was added to tert-butyl ((1S,3S)-3-aminocyclopentyl)carbamate (CAS Reg. No. 645400-44-8) (2.00 g, 9.99 mmol) and K₂CO₃ (2.76 g, 20 mmol) in DMSO (30 mL). The resulting solution was stirred at 125° C. for 18 h under a nitrogen atmosphere. The reaction mixture was diluted with EtOAc (50 mL) and washed with water (3×75 mL). The organic layer was dried over Na₂SO₄, filtered and evaporated. The crude material was purified by flash chromatography on silica (gradient: 0-50% EtOAc in PE) to give the title compound (2.70 g, 67%) as a pale yellow solid. MS (ESI): m/z [M+H]⁺ 403.9.

### Step B. i-1b

### tert-Butyl ((1S,3S)-3-((2-oxo-2H-[1,3′-bipyridin]-6′-yl)amino)cyclopentyl)carbamate

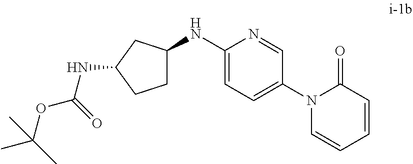 [SMILES: CC(C)(C)OC(=O)N[C@H]1CC[C@H](Nc2ccc(-n3ccccc3=O)cn2)C1]

In a slight modification of GM3 rel-(1R,2R)—N¹,N²-dimethylcyclohexane-1,2-diamine (CAS Reg. No. 67579-81-1) (0.212 g, 1.49 mmol) and Cu(I)I (0.283 g, 1.49 mmol) were added to tert-butyl ((1S,3S)-3-((5-iodopyridin-2-yl)amino)cyclopentyl)carbamate compound i-1a (3.0 g, 7.44 mmol), K₂CO₃ (3.08 g, 22.3 mmol) and pyridin-2 (1H)-one (CAS Reg. No. 142-08-5) (1.42 g, 14.9 mmol) in 1,4-dioxane (20 mL). The resulting solution was stirred at 110° C. for 18 h under a nitrogen atmosphere. The reaction mixture was diluted with EtOAc (25 mL) and washed sequentially with water (3×25 mL). The organic layer was dried over Na₂SO₄, filtered and evaporated. The crude material was triturated with EtOAc:PE (5:1) to give a solid which was collected by filtration and dried under vacuum to give the title compound (2.70 g, 98%) as a yellow solid. MS (ESI): m/z [M+H]⁺ 371.2.

### Step C. i-1c

### 6′-(((1S,3S)-3-Aminocyclopentyl)amino)-2H-[1,3′-bipyridin]-2-one

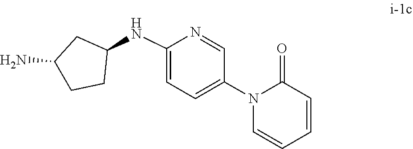 [SMILES: N[C@H]1CC[C@H](Nc2ccc(-n3ccccc3=O)cn2)C1]

In a modification of GM6A HCl (2 M in diethyl ether, 27 mL, 54 mmol) was added slowly to tert-butyl ((1S,3S)-3-((2-oxo-2H-[1,3′-bipyridin]-6′-yl)amino)cyclopentyl)carbamate compound i-1b (1.0 g, 2.70 mmol) in DCM (10 mL) at 25° C. The resulting mixture was stirred at 25° C. for 3 h. This synthesis procedure was repeated for a second batch of tert-butyl ((1S,3S)-3-((2-oxo-2H-[1,3′-bipyridin]-6′-yl)amino)cyclopentyl)carbamate compound i-1b (1.7 g, 4.6 mmol). The two batches were combined and concentrated. The crude product was recrystallised with EtOAc:PE (5:1) to give a solid which was collected by filtration and dried under vacuum to give an unspecified HCl salt of the title compound (2.5 g, 100%) as a yellow solid. MS (ESI): m/z [M+H]⁺ 270.9.

### Intermediate 2

### 1-(6-(((1S,3S)-3-Aminocyclopentyl)amino)pyridin-3-yl)-3-methyl-1,3-dihydro-2H-benzo[d]imidazol-2-one×3 TFA

### Step A. i-2a

### tert-Butyl ((1S,3S)-3-((5-(3-methyl-2-oxo-2,3-dihydro-1H-benzo[d]imidazol-1-yl)pyridin-2-yl)amino)cyclopentyl)carbamate

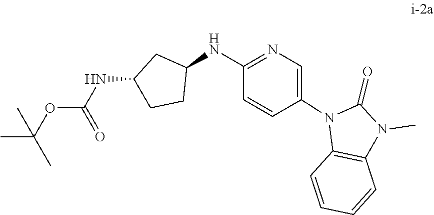 [SMILES: Cn1c(=O)n(-c2ccc(N[C@H]3CC[C@H](NC(=O)OC(C)(C)C)C3)nc2)c2ccccc21]

According to GM3 rel-(1R,2R)—N¹,N²-dimethylcyclohexane-1,2-diamine (88 mg, 0.62 mmol), tert-butyl ((1S,3S)-3-((5-iodopyridin-2-yl)amino)cyclopentyl)carbamate compound i-1a (250 mg, 0.62 mmol), 1-methyl-1,3-dihydro-2H-benzo[d]imidazol-2-one (CAS Reg. No. 1849-01-0) (184 mg, 1.24 mmol), Cs₂CO₃ (606 mg, 1.86 mmol) and Cu(I)I (118 mg, 0.62 mmol) were reacted in 1,4-dioxane (1 mL) at 100° C. for 18 h to give after aqueous work-up and preparative TLC (7 M NH₃ in MeOH:DCM=1:20) the title compound (172 mg, 65%) as a brown gum. MS (ESI): m/z [M+H]⁺ 424.

### Step B. i-2b

### 1-(6-(((1S,3S)-3-Aminocyclopentyl)amino)pyridin-3-yl)-3-methyl-1,3-dihydro-2H-benzo[d]imidazol-2-one×3 TFA

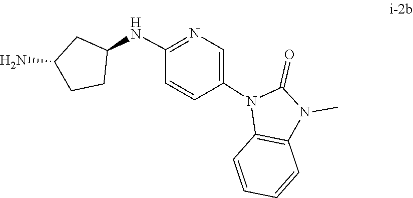 [SMILES: Cn1c(=O)n(-c2ccc(N[C@H]3CC[C@H](N)C3)nc2)c2ccccc21]

In a slight modification of GM6B tert-butyl ((1S,3S)-3-((5-(3-methyl-2-oxo-2,3-dihydro-1H-benzo[d]imidazol-1-yl)pyridin-2-yl)amino)cyclopentyl)carbamate compound i-2a (165 mg, 0.39 mmol) was reacted with TFA (2 mL, 26 mmol) in DCM (4 mL) at 25° C. for 3 h and the reaction mixture filtered through Celite. The filter cake was washed with DCM (2×5 mL) and the filtrate concentrated under reduced pressure to give the title compound (216 mg, 83%) as a brown gum. MS (ESI): m/z [M+H]⁺ 324.

### Intermediate 3

### 6′-(((1S,3S)-3-Aminocyclopentyl)amino)-3-methoxy-2H-[1,3′-bipyridin]-2-one×2 HCl

### Step A. i-3a

### tert-Butyl ((1S,3S)-3-((3-methoxy-2-oxo-2H-[1,3′-bipyridin]-6′-yl)amino)cyclopentyl)carbamate

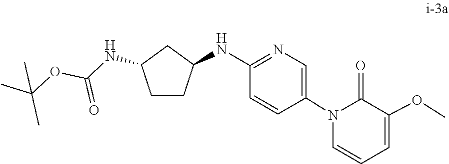 [SMILES: COc1cccn(-c2ccc(N[C@H]3CC[C@H](NC(=O)OC(C)(C)C)C3)nc2)c1=O]

According to GM3 tert-butyl ((1S,3S)-3-((5-iodopyridin-2-yl)amino)cyclopentyl)carbamate compound i-1a (500 mg, 1.24 mmol), 3-methoxypyridin-2 (1H)-one (CAS Reg. No. 20928-63-6) (776 mg, 6.20 mmol), rel-(1R,2R)—N¹,N²-dimethylcyclohexane-1,2-diamine (176 mg, 1.24 mmol), Cs₂CO₃ (2020 mg, 6.20 mmol) and Cu(I)I (236 mg, 1.24 mmol) were reacted in 1,4-dioxane (5 mL) at 100° C. for 15 h to give after aqueous work-up and preparative TLC (MeOH:DCM=1:20) the title compound (387 mg, 78%) as a brown solid. MS (ESI): m/z [M+H]⁺ 401.3.

### Step B. i-3b

### 6′-(((1S,3S)-3-Aminocyclopentyl)amino)-3-methoxy-2H-[1,3′-bipyridin]-2-one×2 HCl

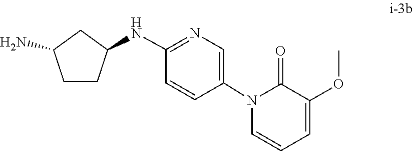 [SMILES: COc1cccn(-c2ccc(N[C@H]3CC[C@H](N)C3)nc2)c1=O]

According to GM6A3 tert-butyl ((1S,3S)-3-((3-methoxy-2-oxo-2H-[1,3′-bipyridin]-6′-yl)amino)cyclopentyl)carbamate compound i-3a (370 mg, 0.92 mmol) was reacted with 4 M HCl in MeOH (10 mL, 40 mmol) in MeOH (15 mL) at 60° C. for 2 h to give the crude title compound (340 mg, 99%) as a brown solid. MS (ESI): m/z [M+H]⁺ 301.1.

### Intermediate 4

### Methyl 6′-(((1S,3S)-3-((5,6-dimethylpyrazin-2-yl)amino)cyclopentyl)amino)-2-oxo-2H-[1,3′-bipyridine]-5-carboxylate

### Step A. i-4a

### tert-Butyl ((1S,3S)-3-((5-nitropyridin-2-yl)amino)cyclopentyl)carbamate

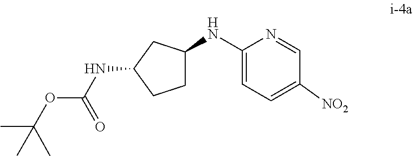 [SMILES: CC(C)(C)OC(=O)N[C@H]1CC[C@H](Nc2ccc([N+](=O)[O-])cn2)C1]

According to GM1A K₂CO₃ (4.14 g, 29.96 mmol), tert-butyl ((1S,3S)-3-aminocyclo-pentyl)carbamate (CAS Reg. No. 645400-44-8) (3.0 g, 14.98 mmol) and 2-chloro-5-nitropyridine (CAS Reg. No. 4548-45-2) (2.375 g, 14.98 mmol) were reacted in DMSO (50 mL) at 100° C. for 3 h. The crude gum obtained upon aqueous work-up was triturated with PE:EtOAc (2:1, 20 mL) to give a solid which was collected by filtration and dried under vacuum to give the title compound (4.0 g, 83%) as a brown solid. MS (ESI): m/z [M+H]⁺ 323.1.

### Step B. i-4b

### tert-Butyl ((1S,3S)-3-((5-aminopyridin-2-yl)amino)cyclopentyl)carbamate

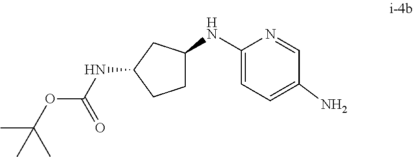 [SMILES: CC(C)(C)OC(=O)N[C@H]1CC[C@H](Nc2ccc(N)cn2)C1]

tert-Butyl ((1S,3S)-3-((5-nitropyridin-2-yl)amino)cyclopentyl)carbamate compound i-4a (1.6 g, 4.96 mmol) and Pd—C (5% wet, 0.53 g, 0.25 mmol) in MeOH (40 mL) were stirred under H₂ (g, 2 atm) at 20° C. for 15 h. The reaction mixture was filtered through Celite and the solvent removed under reduced pressure to give the crude title compound (1.4 g, 96%) as a dark oil which solidified on standing. MS (ESI): m/z [M+H]⁺ 293.0.

### Step C. i-4c

### Methyl 6′-(((1S,3S)-3-((tert-butoxycarbonyl)amino)cyclopentyl)amino)-2-oxo-2H-[1,3′-bipyridine]-5-carboxylate

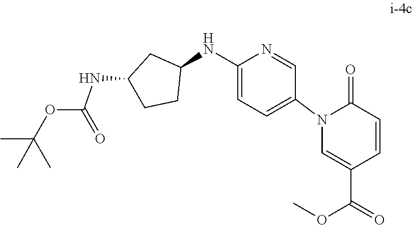 [SMILES: COC(=O)c1ccc(=O)n(-c2ccc(N[C@H]3CC[C@H](NC(=O)OC(C)(C)C)C3)nc2)c1]

Methyl 2-oxo-2H-pyran-5-carboxylate (CAS Reg. No. 6018-41-3) (506 mg, 3.28 mmol) was added to tert-butyl ((1S,3S)-3-((5-aminopyridin-2-yl)amino)cyclopentyl)carbamate compound i-4b (800 mg, 2.74 mmol) in EtOH (20 mL) at 20° C. The resulting solution was stirred at 80° C. for 5 h and the solvent removed under reduced pressure. The crude product was purified by flash chromatography on silica (gradient: 2-30% EtOAc in PE) to give the title compound (800 mg, 68%) as a yellow oil which solidified on standing. MS (ESI): m/z [M+H]⁺ 429.1.

### Step D. i-4d

### Methyl 6′-(((1S,3S)-3-aminocyclopentyl)amino)-2-oxo-2H-[1,3′-bipyridine]-5-carboxylate

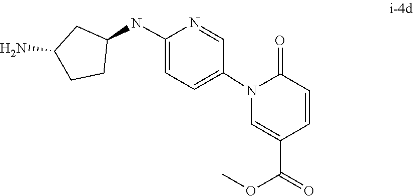

According to GM6A methyl 6′-(((1S,3S)-3-((tert-butoxycarbonyl)amino)cyclopentyl)amino)-2-oxo-2H-[1,3′-bipyridine]-5-carboxylate compound i-4c (800 mg, 1.87 mmol) was reacted with HCl (4 M in MeOH, 6.0 ml, 24 mmol) in MeOH (6 mL) at 60° C. for 2 h to give an unspecified HCl salt of the title compound (661 mg, 97%) as a brown solid. MS (ESI): m/z [M+H]⁺ 329.0.

### Step E. i-4e

### Methyl 6′-(((1S,3S)-3-((5,6-dimethylpyrazin-2-yl)amino)cyclopentyl)amino)-2-oxo-2H-[1,3′-bipyridine]-5-carboxylate

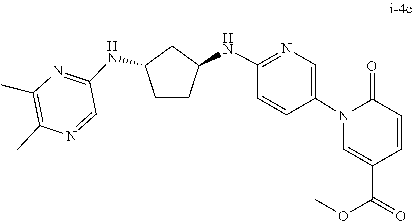 [SMILES: COC(=O)c1ccc(=O)n(-c2ccc(N[C@H]3CC[C@H](Nc4cnc(C)c(C)n4)C3)nc2)c1]

According to GM2 Cs₂CO₃ (893 mg, 2.74 mmol) was added to methyl 6′-(((1S,3S)-3-aminocyclopentyl)amino)-2-oxo-2H-[1,3′-bipyridine]-5-carboxylate compound i-4d (300 mg, 0.91 mmol), 5-chloro-2,3-dimethylpyrazine (CAS Reg. No. 59489-32-6) (261 mg, 1.83 mmol) and Pd-PEPPSI-IpentCl 2-methylpyridine (CAS Reg. No. 1612891-29-8) (38 mg, 0.05 mmol) in 1,4-dioxane (5 mL) at 25° C. The resulting suspension was stirred at 100° C. for 16 h under a nitrogen atmosphere. The reaction mixture was concentrated under reduced pressure, the residue taken up with water (50 mL) and extracted with EtOAc (2×100 mL). The organic layer was dried over Na₂SO₄, filtered and evaporated and the obtained material purified by preparative TLC (7 M NH₃ in MeOH:DCM=1:30) to give the title compound (68 mg, 17%) as a brown solid. MS (ESI): m/z [M+H]⁺ 435.1.

### Intermediate 5

### (1S,3S)—N¹-(5,6-Dimethylpyrazin-2-yl)-N³-(5-iodopyridin-2-yl)cyclopentane-1,3-diamine

### Step A. i-5a

### tert-Butyl ((1S,3S)-3-((5,6-dimethylpyrazin-2-yl)amino)cyclopentyl)carbamate

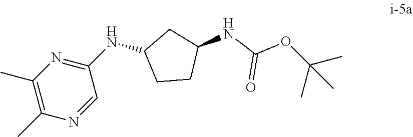

The reaction was run in 3 parallel batches. According to GM2 tert-butyl ((1S,3S)-3-aminocyclopentyl)carbamate (CAS Reg. No. 645400-44-8) (430 mg, 2.15 mmol), 5-chloro-2,3-dimethylpyrazine (CAS Reg. No. 59489-32-6) (306 mg, 2.15 mmol), Cs₂CO₃ (1049 mg, 3.22 mmol) and Pd-PEPPSI-IpentCl 2-methylpyridine (54 mg, 0.06 mmol) were reacted in 1,4-dioxane (20 mL) at 100° C. for 15 h. Upon combination of batches, non-aqueous work-up and trituration (PE:EtOAc=5:1, 25 mL) a solid was collected by filtration which was dried under vacuum to yield the title compound (1.5 g, 75%) as a pale yellow solid. MS (ESI): m/z [M+H]⁺ 307.1.

### Step B. i-5b

### (1S,3S)—N¹-(5,6-Dimethylpyrazin-2-yl)cyclopentane-1,3-diamine×5 TFA

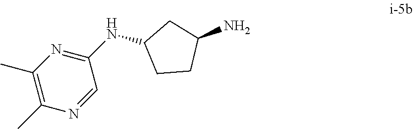

According to GM6B tert-butyl ((1S,3S)-3-((5,6-dimethylpyrazin-2-yl)amino)cyclopentyl)carbamate compound i-5a (1.5 g, 4.9 mmol) was reacted with TFA (4 mL, 52 mmol) in DCM (20 mL) at 20° C. 15 h to give the crude title compound (3.80 g, 100%) as a yellow gum. MS (ESI): m/z [M+H]⁺ 207.2.

### Step C. i-5c

### (1S,3S)—N¹-(5,6-Dimethylpyrazin-2-yl)-N³-(5-iodopyridin-2-yl)cyclopentane-1,3-diamine

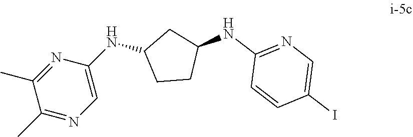

According to GM1A (1S,3S)—N-(5,6-dimethylpyrazin-2-yl)cyclopentane-1,3-diamine×5 TFA compound i-5b (3.80 g, 4.89 mmol), 2-fluoro-5-iodopyridine (CAS Reg. No. 171197-80-1) (2.18 g, 9.79 mmol) and K₂CO₃ (3.38 g, 24.5 mmol) were reacted in DMSO (20 mL) at 120° C. for 15 h to give upon aqueous work-up and purification by flash chromatography on silica (gradient: 2-25% EtOAc in PE) the title compound (1.6 g, 80%) as a pale yellow oil which solidified on standing. MS (ESI): m/z [M+H]⁺ 410.0.

### Intermediate 6

### i-6a

### 1-(6-Chloropyridin-3-yl)-1,8-naphthyridin-2 (1H)-one

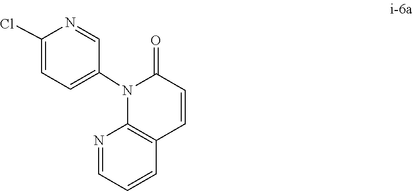 [SMILES: O=c1ccc2cccnc2n1-c1ccc(Cl)nc1]

According to GM5 (6-chloropyridin-3-yl)boronic acid (CAS Reg. No. 444120-91-6) (1.29 g, 8.21 mmol), 1,8-naphthyridin-2 (1H-one (CAS Reg. No. 15936-09-1) (600 mg, 4.11 mmol), N¹,N¹,N²,N²-tetramethylethane-1,2-diamine (1431 mg, 12.32 mmol) and Cu(OTf)₂ (1782 mg, 4.93 mmol) were reacted in 1,4-dioxane (10 mL) at 100° C. for 16 h to give upon purification by C18-flash chromatography (gradient: 5-43% MeCN in water+0.1% NH₃ (aq)) the title compound (252 mg, 23%) as a pale yellow solid. MS (ESI): m/z [M+H]⁺ 257.9.

### Intermediate 7

### 3-(6-Chloropyridin-3-yl)-1-methylimidazolidine-2,4-dione

### Step A. i-7a

### 4-Nitrophenyl (6-chloropyridin-3-yl)carbamate

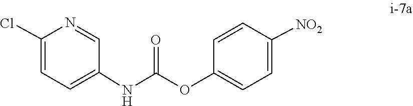 [SMILES: O=C(Nc1ccc(Cl)nc1)Oc1ccc([N+](=O)[O-])cc1]

4-Nitrophenyl carbonochloridate (CAS Reg. No. 7693-46-1) (1.73 g, 8.56 mmol) was added to 6-chloropyridin-3-amine (CAS Reg. No. 5350-93-6) (1.0 g, 7.8 mmol) in MeCN (20 mL) at 20° C. and the resulting solution stirred at this temperature for 30 min. The reaction mixture was diluted with MeCN (200 mL), filtered through an organic phase filter and evaporated to give the crude title compound (2.18 g, 96%) as a purple solid. MS (ESI): m/z [M+H]⁺ 293.9.

### Step B. i-7b

### 3-(6-Chloropyridin-3-yl)-1-methylimidazolidine-2,4-dione

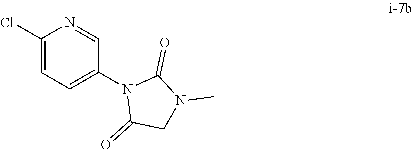

DIPEA (3.90 mL, 22.3 mmol) was added to methyl methylglycinate HCl (CAS Reg. No. 13515-93-0) (1.04 g, 7.44 mmol) in MeCN (20 mL) at 20° C. The resulting suspension was stirred at 20° C. for 15 min upon which 4-nitrophenyl (6-chloropyridin-3-yl)carbamate compound i-7a (2.18 g, 7.44 mmol) was added and stirring at 20° C. continued for 10 min. The mixture was concentrated under reduced pressure and the obtained crude material purified by flash chromatography on silica (gradient: 65-70% EtOAc in PE) to give the title compound (1.55 g, 92%) as a white solid. MS (ESI): m/z [M+H]⁺ 225.8.

### Intermediate 8

### 6′-Chloro-2-oxo-2H-[1,3′-bipyridine]-5-carbonitrile

### Step A. i-8a

### Methyl 6′-chloro-2-oxo-2H-[1,3′-bipyridine]-5-carboxylate

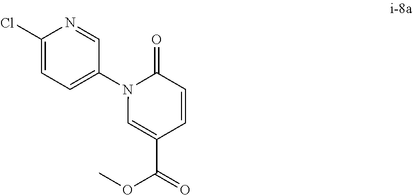 [SMILES: COC(=O)c1ccc(=O)n(-c2ccc(Cl)nc2)c1]

6-Chloropyridin-3-amine (CAS Reg. No. 5350-93-6) (1.0 g, 7.8 mmol) was added to a solution of methyl 2-oxo-2H-pyran-5-carboxylate (CAS Reg. No. 6018-41-3) (1.2 g, 7.8 mmol) in EtOH (20 mL) at 15° C. and it was stirred at 80° C. for 16 h. The reaction mixture was diluted with EtOH (20 mL), filtered through an organic phase filter and evaporated to give the crude title compound (1.53 g, 74%) as a purple solid. MS (ESI): m/z [M+H]⁺ 265.2.

### Step B. i-8b

### 6′-Chloro-2-oxo-2H-[1,3′-bipyridine]-5-carboxylic acid

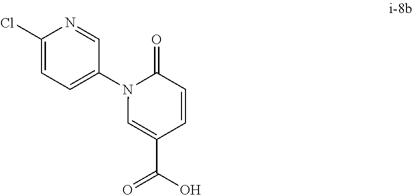 [SMILES: O=C(O)c1ccc(=O)n(-c2ccc(Cl)nc2)c1]

Methyl 6′-chloro-2-oxo-2H-[1,3′-bipyridine]-5-carboxylate compound i-8a (300 mg, 1.13 mmol) was added to NaOH (91 mg, 2.3 mmol) in a mixture of THE (8 mL) and water (2 mL) at 20° C. and the resulting solution stirred at 20° C. for 15 h. The reaction mixture was concentrated und reduced pressure, the residue diluted with water (5 mL) and the pH adjusted to pH<7 with 1 M HCl. The mixture was further diluted with water (100 mL) and extracted with EtOAc (3×75 mL). The organic layer was dried over Na₂SO₄, filtered and evaporated to give crude title compound (230 mg, 81%) as a white solid. MS (ESI): m/z [M+H]⁺ 251.

### Step C. i-8c

### 6′-Chloro-2-oxo-2H-[1,3′-bipyridine]-5-carboxamide

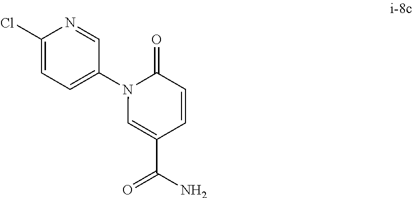 [SMILES: NC(=O)c1ccc(=O)n(-c2ccc(Cl)nc2)c1]

6′-Chloro-2-oxo-2H-[1,3′-bipyridine]-5-carboxylic acid compound i-8b (160 mg, 0.64 mmol) was added to HATU (485 mg, 1.28 mmol), TEA (0.712 mL, 5.11 mmol) and NH₄Cl (137 mg, 2.55 mmol) in DMF (10 mL) under a nitrogen atmosphere. The resulting mixture was stirred at 60° C. for 3 h, subsequently cooled to rt, quenched with sat brine (100 mL) and extracted with EtOAc (3×75 mL). The organic layer was dried over Na₂SO₄, filtered and evaporated and the obtained residue purified by preparative TLC (DCM:MeOH=10:1) to give the title compound (130 mg, 82%) as a white solid. MS (ESI): m/z [M+H]⁺ 250.

### Step D. i-8d

### 6′-Chloro-2-oxo-2H-[1,3′-bipyridine]-5-carbonitrile

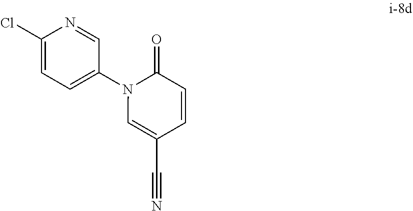 [SMILES: N#Cc1ccc(=O)n(-c2ccc(Cl)nc2)c1]

6′-Chloro-2-oxo-2H-[1,3′-bipyridine]-5-carboxamide compound i-8c (700 mg, 2.80 mmol) was added to a solution of pyridine (0.68 mL, 8.4 mmol) in DCM (10 mL) TFAA (1.19 mL, 8.41 mmol) was slowly added to the mixture at 0° C. and it was stirred at 20° C. for 1 h. The synthesis procedure was repeated with a second batch of 6′-chloro-2-oxo-2H-[1,3′-bipyridine]-5-carboxamide compound i-8c (100 mg, 0.40 mmol). The two batches were combined, concentrated under reduced pressure and purified by preparative TLC (EtOAc:PE=3:1) to give the title compound (540 mg, 72%) as a white solid. MS (ESI): m/z [M+H]⁺ 232.1.

### Intermediate 9

### 3-(Trifluoromethoxy)pyridin-2-ol

### Step A. i-9a

### 2-((4-Methoxybenzyl)oxy)-3-(trifluoromethoxy)pyridine

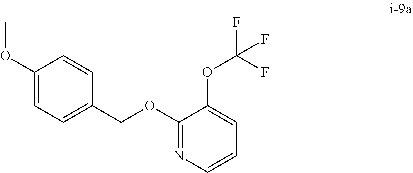 [SMILES: COc1ccc(COc2ncccc2OC(F)(F)F)cc1]

KOtBu (682 mg, 6.07 mmol) was added to (4-methoxyphenyl)methanol (CAS Reg. No. 105-13-5) (629 mg, 4.56 mmol) in 1,4-dioxane (2 mL) at 20° C. and it was stirred at 20° C. for 2 h. 2-Chloro-3-(trifluoromethoxy)pyridine (CAS Reg. No. 1206980-39-3) (600 mg, 3.04 mmol) was added to the reaction mixture and the resulting suspension was stirred at 100° C. for 15 h. The reaction mixture was poured into sat. brine (125 mL) and extracted with EtOAc (3×75 mL). The organic layer was dried over Na₂SO₄, filtered and evaporated and the obtained residue was purified by preparative TLC (EtOAc:PE=1:5) to give the title compound (600 mg, 66%) as a pale yellow liquid. MS (ESI): m/z [M+H]⁺ 300.0.

### Step B. i-9b

### 3-(Trifluoromethoxy)pyridin-2-ol

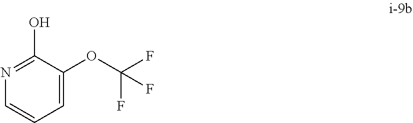 [SMILES: Oc1ncccc1OC(F)(F)F]

2-((4-Methoxybenzyl)oxy)-3-(trifluoromethoxy)pyridine compound i-9a (500 mg, 1.67 mmol) was added to TFA (10 mL) at 25° C., it was warmed to 60° C. and the resulting solution stirred at this temperature for 15 h. The solvent was removed under reduced pressure and the obtained residue purified by preparative TLC (EtOAc:PE=1:1) to give the title compound (150 mg, 50%) as a pale yellow solid. MS (ESI): m/z [M+H]⁺ 180.0.

### Intermediate 10

### i-10a

### 6′-Chloro-2-oxo-2H-[1,3′-bipyridine]-3-carbonitrile

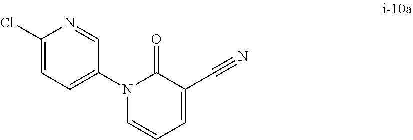 [SMILES: N#Cc1cccn(-c2ccc(Cl)nc2)c1=O]

In a variation of GM5 pyridine (0.81 mL, 10 mmol) was added to a mixture of 2-oxo-1,2-dihydropyridine-3-carbonitrile (CAS Reg. No. 20577-27-9) (600 mg, 5.00 mmol), (6-chloropyridin-3-yl)boronic acid (CAS Reg. No. 444120-91-6) (1.57 g, 10 mmol), Cu(OAc)₂ (1.82 g, 10 mmol) and 5 Å molecular sieves (500 mg, dried 24 h at 200° C.) in DCM (100 mL) and DMF (15 mL) at 25° C. Air was allowed to diffuse into the reaction mixture via a CaCl₂ tube. The resulting mixture was stirred at 25° C. for 15 h and subsequently filtered through Celite. The filter cake was washed with DCM (3×10 mL), the combined filtrates concentrated under reduced pressure and the residue partitioned between EtOAc (400 mL) and water (150 mL). The aqueous layer was extracted with EtOAc (5×200 mL), the combined organic layers were washed with sat. brine (5×100 mL), dried over Na₂SO₄, filtered and evaporated. The dry solid was triturated with DMF (3×5 mL), the solid filtered off and the filter cake washed with MTBE (3×5 mL). The solid was dried under vacuum to give the title compound (105 mg, 9%) as a grey solid. MS (ESI): m/z [M+H]⁺ 232.1.

### Intermediate 13

### i-13a

### 3-Chloro-6′-fluoro-2H-[1,3′-bipyridin]-2-one

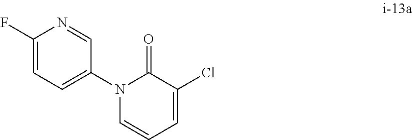 [SMILES: O=c1c(Cl)cccn1-c1ccc(F)nc1]

In a slight variation of GM3 2-fluoro-5-iodopyridine (CAS Reg. No. 171197-80-1) (560 mg, 2.51 mmol), 3-chloropyridin-2 (1H-one (CAS Reg. No. 13466-35-8) (651 mg, 5.02 mmol), potassium phosphate, tribasic (1.60 g, 7.53 mmol), Cu(I)I (478 mg, 2.51 mmol) and rel-(1R,2R)—N¹,N²-dimethylcyclohexane-1,2-diamine (357 mg, 2.51 mmol) were reacted in 1,4-dioxane (60 mL) at 100° C. for 18 h. The reaction mixture was poured into sat. brine (350 mL) and filtered through Celite. The filtrate was extracted with EtOAc (3×250 mL), the organic layer dried over Na₂SO₄, filtered and evaporated. The crude material was purified by flash chromatography on silica (gradient: 0-30% EtOAc in PE) to give the title compound (258 mg, 45%) as a pale yellow solid. MS (ESI): m/z [M+H]⁺ 224.9.

### Intermediate 15

### 6′-(((1S,3S)-3-Aminocyclopentyl)amino)-3-methyl-2H-[1,3′-bipyridin]-2-one×3 HCl

### Step A. i-15a

### tert-Butyl ((1S,3S)-3-((3-methyl-2-oxo-2H-[1,3′-bipyridin]-6′-yl)amino)cyclopentyl)carbamate

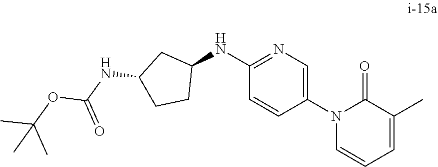 [SMILES: Cc1cccn(-c2ccc(N[C@H]3CC[C@H](NC(=O)OC(C)(C)C)C3)nc2)c1=O]

According to GM3 tert-butyl ((1S,3S)-3-((5-iodopyridin-2-yl)amino)cyclopentyl)carbamate compound i-1a (400 mg, 0.99 mmol), 3-methylpyridin-2 (1H)-one (CAS Reg. No. 1003-56-1) (1.08 g, 9.92 mmol), Cs₂CO₃ (3.23 g, 9.92 mmol), Cu(I)I (378 mg, 1.98 mmol) and rel-(1R,2R)—N¹,N²-dimethylcyclohexane-1,2-diamine (282 mg, 1.98 mmol) were reacted in 1,4-dioxane (15 mL) at 100° C. for 15 h to give after aqueous work-up and preparative TLC (MeOH:DCM=1:15) the title compound (375 mg, 98%) as a yellow solid; MS (ESI): m/z [M+H]⁺ 385.30.

### Step B. i-15b

### 6′-(((1S,3S)-3-Aminocyclopentyl)amino)-3-methyl-2H-[1,3′-bipyridin]-2-one×3 HCl

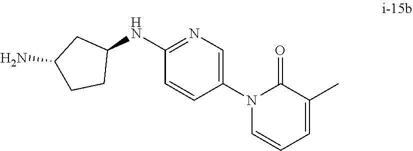 [SMILES: Cc1cccn(-c2ccc(N[C@H]3CC[C@H](N)C3)nc2)c1=O]

According to GM6A tert-butyl ((1S,3S)-3-((3-methyl-2-oxo-2H-[1,3′-bipyridin]-6′-yl)amino)cyclopentyl)carbamate compound i-15a (360 mg, 0.94 mmol) was reacted with HCl in MeOH (4 M, 10 mL, 40 mmol) in MeOH (15 mL) at 60° C. for 2 h to afford the title compound (356 mg, 97%) as a yellow solid. MS (ESI): m/z [M+H]⁺ 285.20.

### Intermediate 16

### 2-(6-(((1S,3S)-3-Aminocyclopentyl)amino)pyridin-3-yl)pyridazin-3 (2H)-one

### Step A. i-16a

### tert-Butyl ((1S,3S)-3-((5-(6-oxopyridazin-1 (6H)-yl)pyridin-2-yl)amino)cyclopentyl)carbamate

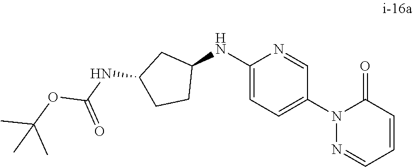 [SMILES: CC(C)(C)OC(=O)N[C@H]1CC[C@H](Nc2ccc(-n3ncccc3=O)cn2)C1]

In a slight modification of GM3 tert-butyl ((1S,3S)-3-((5-iodopyridin-2-yl)amino)cyclopentyl)carbamate compound i-1a (250 mg, 0.62 mmol), pyridazin-3 (2H)-one (CAS Reg. No. 504-30-3) (119 mg, 1.24 mmol), Cs₂CO₃ (606 mg, 1.86 mmol), Cu(I)I (59 mg, 0.31 mmol) and N¹,N²-dimethylethane-1,2-diamine (27 mg, 0.31 mmol) were reacted in 1,4-dioxane (5 mL) at 100° C. for 16 h to give after aqueous work-up and preparative TLC (NH₃ in MeOH (7 M):DCM=1:30) the title compound (173 mg, 75%) as a brown solid. MS (ESI): m/z (ESI), [M+H]⁺ 372.0.

### Step B. i-16b

### 2-(6-(((1S,3S)-3-Aminocyclopentyl)amino)pyridin-3-yl)pyridazin-3 (2H)-one

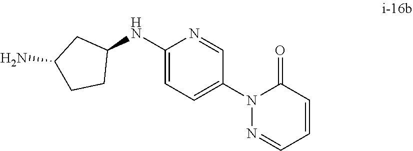 [SMILES: N[C@H]1CC[C@H](Nc2ccc(-n3ncccc3=O)cn2)C1]

According to GM6A tert-butyl ((1S,3S)-3-((5-(6-oxopyridazin-1 (6H)-yl)pyridin-2-yl)amino)cyclopentyl)carbamate compound i-16a (168 mg, 0.45 mmol) was reacted with HCl in MeOH (4 M, 5.0 mL, 20 mmol) in MeOH (5 mL) at 60° C. for 3 h to afford an unspecified HCl salt of the title compound (156 mg, 100%) as a brown solid. MS (ESI): m/z [M+H]⁺ 272.00.

### Intermediate 17

### (1S,3S)—N¹-(5-Chloropyrazin-2-yl)-M-(5-iodopyridin-2-yl)cyclopentane-1,3-diamine

### Step A. i-17a

### (1S,3S)—N¹-(5-iodopyridin-2-yl)cyclopentane-1,3-diamine×3 HCl

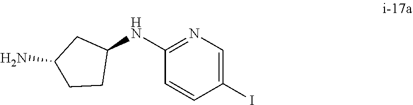 [SMILES: N[C@H]1CC[C@H](Nc2ccc(I)cn2)C1]

According to GM6A tert-butyl ((1S,3S)-3-((5-iodopyridin-2-yl)amino)cyclopentyl)carbamate compound i-1a (Intermediate 1, Step A) (1.29 g, 3.19 mmol) was reacted with HCl in MeOH (4 M, 10 mL, 40 mmol) in MeOH (20 mL) at 80° C. for 2 h to afford the title compound (1.27 g, 96%) as a beige solid. MS (ESI): m/z [M+H]⁺ 303.90.

### Step B. i-17b

### (1S,3S)—N¹-(5-Chloropyrazin-2-yl)-M-(5-iodopyridin-2-yl)cyclopentane-1,3-diamine

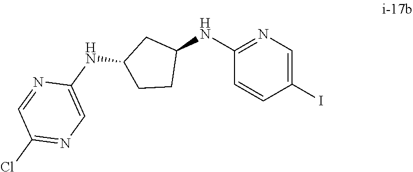 [SMILES: Clc1cnc(N[C@H]2CC[C@H](Nc3ccc(I)cn3)C2)cn1]

According to GM1A (1S,3S)—N¹-(5-iodopyridin-2-yl)cyclopentane-1,3-diamine compound i-17a (1.26 g, 3.05 mmol), 2,5-dichloropyrazine (CAS Reg. No. 19745-07-4) (0.910 g, 6.11 mmol) and Na₂CO₃ (1.62 g, 15.3 mmol) were reacted in DMSO (25 mL) at 120° C. for 15 h. The mixture was filtered through a Celite pad and the filtrate purified by flash C18-flash chromatography (gradient: 10-61% MeCN in water, containing 0.1% NH₃, aq) to give the title compound (0.78 g, 61%) as a beige solid. MS (ESI): m/z [M+H]⁺ 415.90.

### Intermediate 18

### i-18a

### Methyl 6′-(((1S,3S)-3-((5-chloropyrazin-2-yl)amino)cyclopentyl)amino)-2-oxo-2H-[1,3′-bipyridine]-5-carboxylate

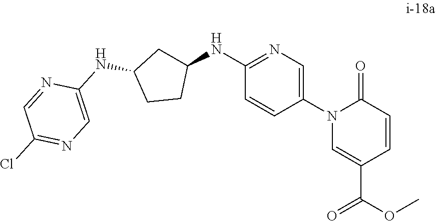 [SMILES: COC(=O)c1ccc(=O)n(-c2ccc(N[C@H]3CC[C@H](Nc4cnc(Cl)cn4)C3)nc2)c1]

According to GM1A methyl 6′-(((1S,3S)-3-aminocyclopentyl)amino)-2-oxo-2H-[1,3′-bipyridine]-5-carboxylate compound i-4d (185 mg, 0.51 mmol), 2,5-dichloropyrazine (CAS Reg. No. 19745-07-4) (151 mg, 1.01 mmol) and Na₂CO₃ (161 mg, 1.52 mmol) were reacted in DMSO (5 mL) at 120° C. for 3 h to give upon aqueous work-up and purification by preparative TLC (NH₃ in MeOH:DCM=1:20) the title compound (93 mg, 42%) as a brown solid. MS (ESI): m/z [M+H]⁺ 441.05.

### Intermediate 19

### (1S,3S)—N¹-(5-(difluoromethoxy)pyrazin-2-yl)-N3-(5-iodopyridin-2-yl)cyclopentane-1,3-diamine

### Step A. i-19a

### 2-Chloro-5-(difluoromethoxy)pyrazine

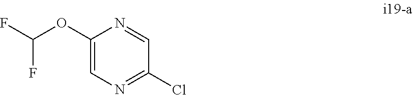 [SMILES: FC(F)Oc1cnc(Cl)cn1]

At 10° C. diethyl (bromodifluoromethyl)phosphonate (CAS Reg. No. 65094-22-6) (80 g, 299 mmol) was added slowly to a mixture of 5-chloropyrazin-2-ol (CAS Reg. No. 89180-45-0) (13 g, 99.6 mmol) and aqueous potassium hydroxide (6N) (180 mL, 1080 mmol) in acetonitrile (180 mL). After stirring the resulting solution at 20° C. for 30 minutes the solvent was removed under reduced pressure. The residue was poured into saturated brine (400 mL), extracted with EtOAc (3×200 mL), the organic layer was dried over Na₂SO₄, filtered and evaporated to afford brown liquid. The crude product was purified by flash chromatography on silica (gradient: 0-2% EtOAc in PE) to afford the title compound (9.00 g, 50%) as a pale yellow liquid. ¹H-NMR (300 MHz, CDCl₃) δ ppm 7.36 (1H, t), 8.21 (2H, s).

### Step B. i-19b

### tert-Butyl ((1S,3S)-3-((5-(difluoromethoxy)pyrazin-2-yl)amino)cyclopentyl)carbamate

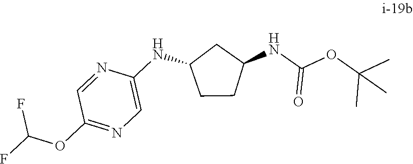 [SMILES: CC(C)(C)OC(=O)N[C@H]1CC[C@H](Nc2cnc(OC(F)F)cn2)C1]

According to GM2 2-chloro-5-(difluoromethoxy)pyrazine compound i-19a (1.0 g, 5.54 mmol), tert-butyl ((1S,3S)-3-aminocyclopentyl)carbamate (CAS Reg. No. 645400-44-8) (1.24 g, 6.20 mmol), Cs₂CO₃ (3.6 g, 11 mmol) and Pd-PEPPSI-IpentCl 2-methylpyridine (0.19 g, 0.22 mmol) were reacted in 1,4-dioxane (30 mL) at 100° C. for 16 h to give upon non-aqueous workup the title compound (1.6 g, 84%) as a pale yellow solid. MS (ESI): m/z [M+H]⁺ 345.05. ¹H-NMR (300 MHz, CDCl₃) δ ppm 1.46 (11H, s), 1.95 (2H, t), 2.26 (2H, dddd), 4.09-4.25 (2H, m), 6.98 (1H, d), 7.47 (1H, d), 7.88 (1H, d).

### Step C. i-19c

### (1S,3S)—N¹-(5-(Difluoromethoxy)pyrazin-2-yl)cyclopentane-1,3-diamine×2 TFA

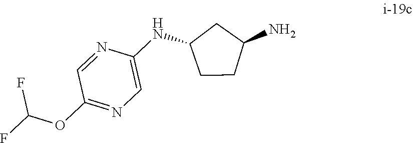 [SMILES: N[C@H]1CC[C@H](Nc2cnc(OC(F)F)cn2)C1]

According to GM6B tert-butyl ((1S,3S)-3-((5-(difluoromethoxy)pyrazin-2-yl)amino)cyclopentyl)carbamate compound i-19b (1.55 g, 4.50 mmol) was deprotected with TFA (10 mL, 130 mmol) in DCM (20 mL) for 16 h to give the title compound (2.0 g, 94%) as a brown gum. MS (ESI): m/z [M-NH₃+H]⁺227.95. ¹H-NMR (300 MHz, MeOH-d₄) δ ppm 1.59-1.76 (2H, m), 1.98-2.12 (2H, m), 2.21-2.36 (2H, m), 3.75 (1H, q), 4.29-4.40 (1H, m), 7.15 (1H, t), 7.56 (1H, d), 7.80 (1H, d).

### Step D. i-19d

### (1S,3S)—N¹-(5-(difluoromethoxy)pyrazin-2-yl)-N3-(5-iodopyridin-2-yl)cyclopentane-1,3-diamine

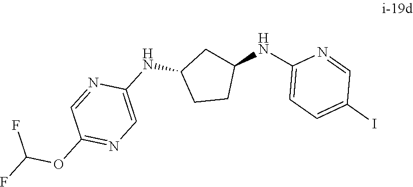 [SMILES: FC(F)Oc1cnc(N[C@H]2CC[C@H](Nc3ccc(I)cn3)C2)cn1]

According to GM1A (1S,3S)—N¹-(5-(difluoromethoxy)pyrazin-2-yl)cyclopentane-1,3-diamine compound i-19c (1.27 g, 2.69 mmol), 2-fluoro-5-iodo pyridine (CAS Reg. No. 171197-80-1) (500 mg, 2.24 mmol) and Cs₂CO₃ (1.46 g, 4.48 mmol) were reacted in DMSO (5 mL) at 100° C. for 16 h to give upon aqueous work-up the title compound (1.03 mmol, 46%) as a yellow solid. HRMS (ESI) m/z [M+H]⁺ calcd for C₁₅H₁₇F₂IN₅O: 448.0440, found: 448.0412. ¹H NMR (300 MHz, MeOH-d₄) δ 1.49-1.62 (2H, m), 1.95 (2H, t), 2.16-2.31 (2H, m), 4.18-4.33 (2H, m), 6.39 (1H, dd), 7.25 (1H, d), 7.53-7.61 (2H, m), 7.77 (1H, d), 8.07 (1H, dd).

### Intermediate 20

### 1-(6-(((1S,3S)-3-Aminocyclopentyl)amino)pyridin-3-yl)-1,8-naphthyridin-2 (1H)-one

### Step A. i-20a

### tert-Butyl ((1S,3S)-3-((5-(2-oxo-1,8-naphthyridin-1 (2H)-yl)pyridin-2-yl)amino)cyclopentyl)carbamate

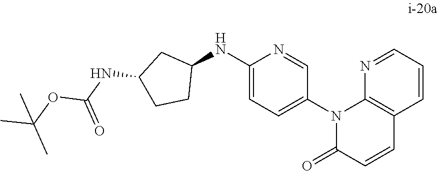 [SMILES: CC(C)(C)OC(=O)N[C@H]1CC[C@H](Nc2ccc(-n3c(=O)ccc4cccnc43)cn2)C1]

According to GM2 1-(6-chloropyridin-3-yl)-1,8-naphthyridin-2 (1H-one compound i-6a (100 mg, 0.39 mmol), tert-butyl ((1S,3S)-3-aminocyclopentyl)carbamate (CAS Reg. No. 645400-44-8) (117 mg, 0.58 mmol), Cs₂CO₃ (379 mg, 1.16 mmol) and Pd-PEPPSI-IpentCl 2-methylpyridine (33 mg, 0.04 mmol) were reacted in 1,4-dioxane (5 mL) at 100° C. for 18 h to give upon aqueous work-up and purification by preparative TLC (MeOH:DCM=1:30) the title compound (140 mg, 86%) as a pale yellow solid. MS (ESI): m/z [M+H]⁺ 422.10.

### Step B. i-20b

### 1-(6-(((1S,3S)-3-Aminocyclopentyl)amino)pyridin-3-yl)-1,8-naphthyridin-2 (1H)-one

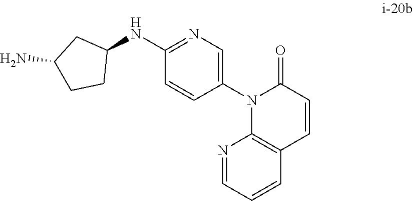 [SMILES: N[C@H]1CC[C@H](Nc2ccc(-n3c(=O)ccc4cccnc43)cn2)C1]

According to GM6A tert-butyl ((1S,3S)-3-((5-(2-oxo-1,8-naphthyridin-1 (2H)-yl)pyridin-2-yl)amino)cyclopentyl)carbamate compound i-20a (130 mg, 0.31 mmol) was reacted with HCl in methanol (4 M 5.0 mL, 20 mmol) in MeOH (5 mL) at 80° C. for 2 h to give an unspecified HCl salt of the title compound (105 mg, 95%) as a light brown solid. MS (ESI): m/z [M+H]⁺ 322.00.

### Intermediate 21

### 1-(6-(((1S,3S)-3-Aminocyclopentyl)amino)pyridin-3-yl)quinolin-2 (1H)-one

### Step A. i-21a

### 1-(6-Chloropyridin-3-yl)quinolin-2 (1H)-one

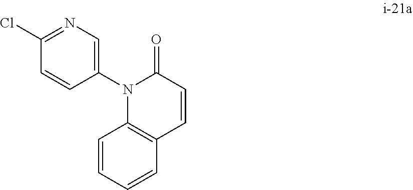 [SMILES: O=c1ccc2ccccc2n1-c1ccc(Cl)nc1]

According to GM5 quinolin-2 (1H-one (CAS Reg. No. (59-31-4) (500 mg, 3.44 mmol), (6-chloropyridin-3-yl)boronic acid (CAS Reg. No. (444120-91-6) (1.63 g, 10.3 mmol), Cu(OTf)₂ (2.49 g, 6.89 mmol) and N,N,N′,N′-tetramethylethylenediamine (1.20 g, 10.3 mmol) were reacted in 1,4-dioxane (20 mL) at 100° C. for 16 h to give upon purification by C18-flash chromatography (gradient: 5-46% MeCN in water, containing 0.1% NH₃, aq) 1-(6-chloropyridin-3-yl)quinolin-2 (1H-one (211 mg, 24%) as a green solid. MS (ESI): m/z [M+H]⁺ 256.95.

### Step B. i-21b

### tert-Butyl ((1S,3S)-3-((5-(2-oxoquinolin-1 (2H)-yl)pyridin-2-yl)amino)cyclopentyl)carbamate

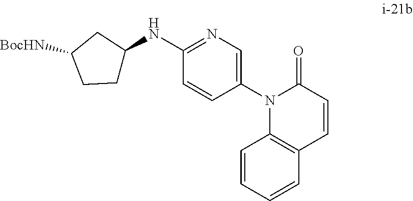 [SMILES: CC(C)(C)OC(=O)N[C@H]1CC[C@H](Nc2ccc(-n3c(=O)ccc4ccccc43)cn2)C1]

According to GM2 1-(6-chloropyridin-3-yl)quinolin-2 (1H)-one compound i-21a (90 mg, 0.35 mmol), tert-butyl ((1S,3S)-3-aminocyclopentyl)carbamate (CAS Reg. No. 645400-44-8) (211 mg, 1.05 mmol), Cs₂CO₃ (571 mg, 1.75 mmol) and Pd-PEPPSI-IpentCl 2-methylpyridine (29.5 mg, 0.04 mmol) were reacted in 1,4-dioxane (10 mL) at 100° C. for 15 h to give upon aqueous work-up and purification by preparative TLC (MeOH:DCM=1:40) tert-butyl ((1S,3S)-3-((5-(2-oxoquinolin-1 (2H)-yl)pyridin-2-yl)amino)cyclopentyl)carbamate (122 mg, 83%) as a white solid. MS (ESI): m/z [M+H]⁺ 421.00.

### Step C. i-21c

### 1-(6-(((1S,3S)-3-Aminocyclopentyl)amino)pyridin-3-yl)quinolin-2 (1H)-one

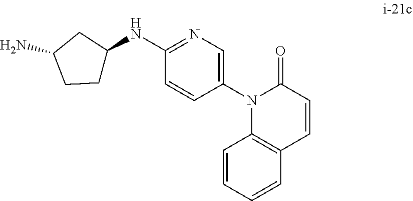 [SMILES: N[C@H]1CC[C@H](Nc2ccc(-n3c(=O)ccc4ccccc43)cn2)C1]

According to GM6A tert-butyl ((1S,3S)-3-((5-(2-oxoquinolin-1 (2H)-yl)pyridin-2-yl)amino)cyclopentyl)carbamate compound i-21b (110 mg, 0.26 mmol) was reacted with HCl in methanol (4 M, 5.0 mL, 20 mmol) in MeOH (10 mL) at 80° C. for 2 h to afford an unspecified HCl salt of 1-(6-(((1S,3S)-3-aminocyclopentyl)amino)pyridin-3-yl)quinolin-2 (1H-one (90 mg, 96%) as a brown gum. MS (ESI): m/z [M+H]⁺ 321.00.

### Intermediate 22

### (1S,3S)—N¹-(6-Cyclopropyl-1,2,4-triazin-3-yl)-AP-(5-iodopyridin-2-yl)cyclopentane-1,3-diamine

### Step A. i-22a

### tert-Butyl ((1S,3S)-3-((1,2,4-triazin-3-yl)amino)cyclopentyl)carbamate

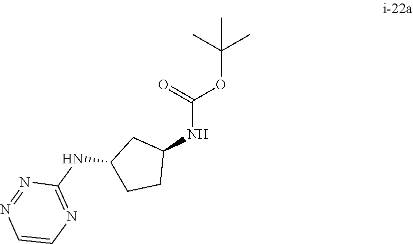 [SMILES: CC(C)(C)OC(=O)N[C@H]1CC[C@H](Nc2nccnn2)C1]

m-CPBA (5.60 g, 25.95 mmol) was added in small portions to a solution of 3-(methylthio)-1,2,4-triazine (CAS Reg. No. 28735-21-9) (3.0 g, 23.6 mmol) in DCM (80 mL) at 0° C. and the resulting suspension was stirred at 20° C. for 2 h. The solvent was removed under reduced pressure without heating, the residue dissolved in n-butanol (40 mL) and tert-butyl ((1S,3S)-3-aminocyclopentyl)carbamate (CAS Reg. No. 645400-44-8) (5.20 g, 26.0 mmol) was added and the resulting solution was stirred at 120° C. for 18 h. The reaction mixture was poured into 1 M NaOH (250 mL) and extracted with EtOAc (3×200 mL). The combined organic layers were dried (Na₂SO₄), filtered and evaporated to afford the crude product as a brown solid which was purified by flash chromatography on silica (gradient: 5-60% EtOAc in PE) to give (3.7 g, 56%) of the title compound as a yellow solid. MS (ESI): m/z [M+H]⁺ 280.

### Step B. i-22b

### tert-Butyl ((1S,3S)-3-((6-bromo-1,2,4-triazin-3-yl)amino)cyclopentyl)carbamate

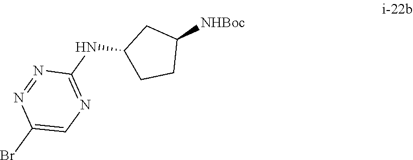

A solution of Br₂ in DCM (1 M, 15.9 mL, 15.9 mmol) was added dropwise to a solution of tert-butyl ((1S,3S)-3-((1,2,4-triazin-3-yl)amino)cyclopentyl)carbamate compound i-22a (3.7 g, 13.2 mmol) in a mixture of MeOH (60 mL) and water (30 mL) and the mixture was stirred at rt for 15 h. The solvent was removed under reduced pressure and the residue was poured into sat. Na₂SO₃ (aq) (150 mL) and extracted with EtOAc (3×100 mL). The organic layer was dried (Na₂SO₄), filtered and evaporated to afford the crude product as brown solid which was purified by flash chromatography on silica (gradient: 5-30% EtOAc in PE) to give (3.5 g, 74%) of the title compound as a yellow solid. MS (ESI): m/z [M+H]⁺ 358.

### Step C. i-22c

### tert-Butyl ((1S,3S)-3-((6-cyclopropyl-1,2,4-triazin-3-yl)amino)cyclopentyl)carbamate

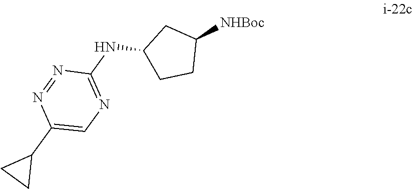

According to GM4B tert-butyl ((1S,3S)-3-((6-bromo-1,2,4-triazin-3-yl)amino)cyclopentyl)carbamate compound i-22b (1.5 g, 4.2 mmol), potassium cyclopropyltrifluoroborate (2.48 g, 16.8 mmol), Cs₂CO₃ (5.46 g, 16.8 mmol) and CataCXium A Pd G3 (CAS Reg. No. 1651823-59-4) (0.61 g, 0.84 mmol) were reacted in dioxane (70 mL) at 100° C. for 15 h. Upon combination with a second batch (prepared in the same manner, 1.12 mmol scale) the material was purified by preparative TLC (EtOAc:PE=1:3) to afford (1.12 g, 66%) of the title compound as a pale yellow solid. MS (ESI): m/z [M+H]⁺ 320.

### Step D. i-22d

### (1S,3S)—N¹-(6-Cyclopropyl-1,2,4-triazin-3-yl)cyclopentane-1,3-diamine×4 TFA

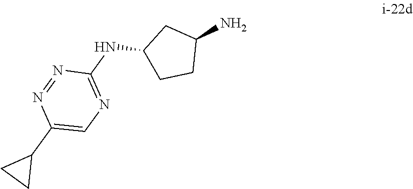 [SMILES: N[C@H]1CC[C@H](Nc2ncc(C3CC3)nn2)C1]

According to GM6B tert-butyl ((1S,3S)-3-((6-cyclopropyl-1,2,4-triazin-3-yl)amino)cyclopentyl)carbamate compound i-22c (500 mg, 1.57 mmol) was reacted with TFA (5.0 mL, 65 mmol) in DCM (20 mL) at rt for 15 h to give (940 mg, 89%) of the title compound as a yellow gum (contains 4 mol eq of residual TFA). MS (ESI): m/z [M+H]⁺ 220.

### Step E. i-22e

### (1S,3S)—N¹-(6-Cyclopropyl-1,2,4-triazin-3-yl)-NM-(5-iodopyridin-2-yl)cyclopentane-1,3-diamine

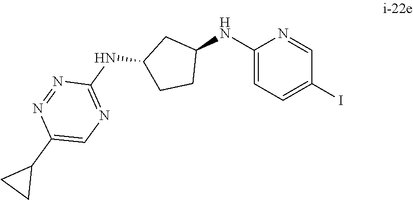 [SMILES: Ic1ccc(N[C@H]2CC[C@H](Nc3ncc(C4CC4)nn3)C2)nc1]

According to GM1A (1S,3S)—N¹-(6-cyclopropyl-1,2,4-triazin-3-yl)cyclopentane-1,3-diamine 4TFA compound i-22d (880 mg, 1.30 mmol), 2-fluoro-5-iodopyridine (436 mg, 1.95 mmol) and K₂CO₃ (900 mg, 6.51 mmol) were reacted in DMSO (10 mL) at 120° C. for 15 h to give upon aqueous work-up and purification by preparative TLC (EtOAc:PE=3:1) the title compound (369 mg, 67%) as a pale yellow solid. MS (ESI): m/z [M+H]⁺ 423.

### Intermediate 23

### i-23a

### 1-(6-Chloropyridin-3-yl)-3-methylimidazolidine-2,4-dione

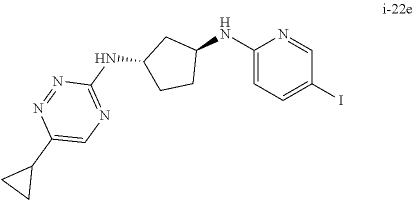 [SMILES: Ic1ccc(N[C@H]2CC[C@H](Nc3ncc(C4CC4)nn3)C2)nc1]

CuI (0.230 g, 1.57 mmol) was added to a mixture of 2-chloro-5-iodopyridine (CAS Reg. No. 69045-79-0) (0.50 g, 2.09 mmol), 3-methylimidazolidine-2,4-dione (CAS Reg. No. 6843-45-4) (0.48 g, 4.18 mmol), N,N-dimethylglycine hydrochloride (0.22 g, 1.57 mmol) and Cs₂CO₃ (1.36 g, 4.18 mmol) in dioxane (20 mL) and the resulting suspension was stirred at 100° C. for 15 h under nitrogen. The reaction mixture was poured into brine (125 mL) and extracted with EtOAc (3×100 mL). The organic layer was dried (Na₂SO₄), filtered and evaporated to afford the crude product as yellow gum which was purified by preparative TLC (EtOAc:PE=1:1) to give (0.25 g, 53%) of the title compound as a pale yellow oil which solidified on standing. MS (ESI): m/z [M+H]⁺ 226.

### Intermediate 24

### i-24a

### 3-(6-Chloropyridin-3-yl)-1-methyl-1,3-dihydro-2H-imidazo[4,5-b]pyridin-2-one

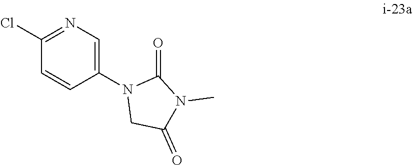

According to GM5 (6-chloropyridin-3-yl)boronic acid (CAS Reg. No. 444120-91-6) (2.93 g, 18.6 mmol), 1-methyl-1,3-dihydro-2H-imidazo[4,5-b]pyridin-2-one (CAS Reg. No. 50339-06-5) (1.11 g, 7.44 mmol), Cu(OTf)₂ (4.04 g, 11.2 mmol) and TMEDA (2.59 g, 22.3 mmol) were reacted in dioxane (100 mL) at 100° C. for 16 h to give upon work-up and purification by C18-flash chromatography (gradient: 5-40% MeCN in water, containing 0.1% NH₃, aq) (0.235 g, 12%) of the title compound as a white solid. MS (ESI): m/z [M+H]⁺ 261.

### Intermediate 25

### i-25a

### 6′-Chloro-3-methoxy-2H-[1,3′-bipyridin]-2-one

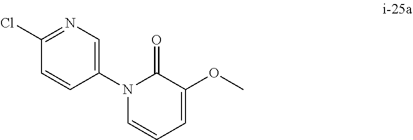 [SMILES: COc1cccn(-c2ccc(Cl)nc2)c1=O]

3-Methoxypyridin-2 (1H)-one (CAS Reg. No. 20928-63-6) (1.35 g, 10.8 mol) and 2-chloro-5-iodopyridine (CAS Reg. No. 69045-72-0) (1.03 g, 4.32 mmol) were added to dimethylglycine hydrochloride (301 mg, 2.16 mmol), CuI (411 mg, 2.16 mmol) and Cs₂CO₃ (4.22 g, 13.0 mmol) in dioxane (20 mL) and the resulting solution was stirred at 100° C. for 15 h. The solvent was removed under reduced pressure and the crude product was purified by preparative TLC (PE:EtOAc=1:2) to afford (250 mg, 55%) of the title compound as a pale yellow solid. MS (ESI): m/z [M+H]⁺ 237.

### Intermediate 26 i-26a

### 6′-Chloro-5-methoxy-2H-[1,3′-bipyridin]-2-one

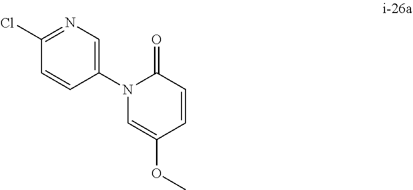 [SMILES: COc1ccc(=O)n(-c2ccc(Cl)nc2)c1]

CuI (159 mg, 0.84 mmol) and N,N-dimethylglycine hydrochloride (117 mg, 0.84 mmol) were added to a mixture of 2-chloro-5-iodopyridine (400 mg, 1.67 mmol), 5-methoxypyridin-2 (1H-one (CAS Reg. No. 61941-79-5) (523 mg, 4.18 mmol) and Cs₂CO₃ (1.63 mg, 5.01 mmol) in dioxane (8 mL) and the resulting suspension was stirred at 100° C. for 15 h under nitrogen. The mixture was filtered through a Celite pad, the solvent removed under reduced pressure and the crude product purified by preparative TLC (EtOAc:PE=3:1) to afford (179 mg, 45%) of the title compound as a white solid. MS (ESI): m/z [M+H]⁺ 237.

### Intermediate 28

### (1S,3S)—N¹-(6-Methyl-1,2,4-triazin-3-yl)cyclopentane-1,3-diamine×2 TFA

### Step A. i-28a

### tert-Butyl ((1S,3S)-3-((6-methyl-1,2,4-triazin-3-yl)amino)cyclopentyl)carbamate

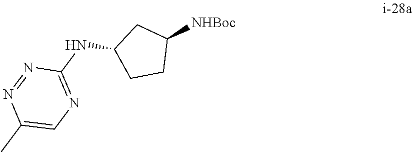

According to GM4A tert-butyl ((1S,3S)-3-((6-bromo-1,2,4-triazin-3-yl)amino)cyclopentyl)carbamate compound i-22b (300 mg, 0.84 mmol), 2,4,6-trimethyl-1,3,5,2,4,6-trioxatriborinane in THE (2.10 g, 8.37 mmol), K₃PO₄ (356 mg, 1.67 mmol) and 1,1′-bis(di-tert-butylphosphino)ferrocene palladium dichloride (CAS Reg. No. 9548-5-) (55 mg, 0.08 mmol) were reacted in a mixture of dioxane (4 mL) and water (1 mL) at 100° C. for 15 h to give upon aqueous work-up and purification by preparative TLC (MeOH:DCM=1:20) followed by C18-flash chromatography (gradient: 0-49% MeOH in water) the title compound (103 mg, 42%) as a brown solid. MS (ESI): m/z [M+H]⁺ 294.

### Step B. i-28b

### (1S,3S)—N¹-(6-Methyl-1,2,4-triazin-3-yl)cyclopentane-1,3-diamine×2 TFA

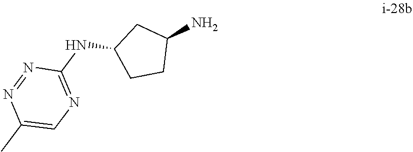

According to GM6B tert-butyl ((1S,3S)-3-((6-methyl-1,2,4-triazin-3-yl)amino)cyclopentyl)carbamate compound i-28a (100 mg, 0.34 mmol) was reacted with TFA (5 mL) in DCM (5 mL) at rt for 15 h to afford (100 mg, 95%) of title compound as a white solid. MS (ESI): m/z [M+H]⁺ 194.

### Intermediate 31

### (1S,3S)—N¹-(5-Methylpyrazin-2-yl)cyclopentane-1,3-diamine

### Step A. i-31a

### tert-Butyl ((1S,3S)-3-((5-methylpyrazin-2-yl)amino)cyclopentyl)carbamate

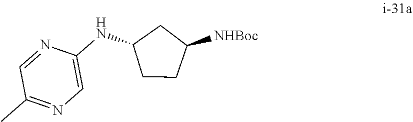 [SMILES: Cc1cnc(N[C@H]2CC[C@H](NC(=O)OC(C)(C)C)C2)cn1]

The reaction was run in 3 parallel batches.

According to GM2 tert-butyl ((1S,3S)-3-aminocyclopentyl)carbamate (CAS Reg. No. 645400-44-8) (420 mg, 2.10 mmol), 2-bromo-5-methylpyrazine (CAS Reg. No. 98006-90-7) (363 mg, 2.10 mmol), Cs₂CO₃ (1.03 g, 3.15 mmol) and Pd-PEPPSI-IpentCl 2-methylpyridine (62 mg, 0.07 mmol) were reacted in 1,4-dioxane (20 mL) at 100° C. for 15 h. Upon combination of batches, non-aqueous work-up and trituration (PE:EtOAc=2:1, 20 mL) a solid was collected by filtration which was dried under vacuum to yield the title compound (1.2 g, 63%) as a grey solid. MS (ESI): m/z [M+H]⁺ 293.3. ¹H NMR (300 MHz, CDCl₃) δ ppm 1.46 (11H, s), 1.93 (2H, td), 2.25 (2H, ddt), 2.39 (3H, d), 4.15 (1H, d), 4.23 (1H, p), 4.47 (1H, d), 4.57 (1H, s), 7.80 (1H, d), 7.85-7.90 (1H, m).

### Step B. i-31b

### (1S,3S)—N¹-(5-Methylpyrazin-2-yl)cyclopentane-1,3-diamine

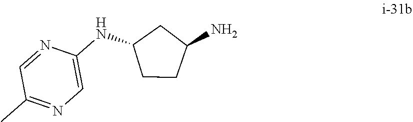

According to GM6B tert-butyl ((1S,3S)-3-((5-methylpyrazin-2-yl)amino)cyclopentyl)carbamate compound i-31a (350 mg, 1.20 mmol) was reacted with TFA (2 mL, 26 mmol) in DCM (10 mL) at 20° C. for 15 h to afford an unspecified TFA salt of the title compound (704 mg, 91%) as a brown gum. MS (ESI): m/z [M+H]⁺ 193.0. ¹H NMR (300 MHz, DMSO-d₆) δ ppm 1.44-1.64 (2H, m), 1.91 (2H, m), 2.06-2.19 (2H, m), 2.26 (3H, s), 3.60-3.73 (1H, m), 4.21-4.32 (1H, m), 7.74-7.94 (5H, m).

### Intermediate 32

### i-32a

### (1S,3S)—N¹-(5-Iodopyridin-2-yl)-NM-(5-methylpyrazin-2-yl)cyclopentane-1,3-diamine

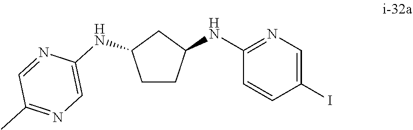 [SMILES: Cc1cnc(N[C@H]2CC[C@H](Nc3ccc(I)cn3)C2)cn1]

According to GM1A (1S,3S)—N¹-(5-methylpyrazin-2-yl)cyclopentane-1,3-diamine compound i-31b (1.65 g, 2.54 mmol), 2-fluoro-5-iodopyridine (CAS Reg. No. 171197-80-1) (1.14 g, 5.09 mmol) and K₂CO₃ (2.11 g, 15.3 mmol) were reacted in DMSO (20 mL) at 120° C. for 15 h to give upon aqueous work-up and purification by flash chromatography on silica (gradient: 2-25% EtOAc in PE) the title compound (0.70 g, 70%) as a pale-yellow oil which solidified on standing. MS (ESI): m/z [M+H]⁺ 396.15. ¹H NMR (300 MHz, CDCl₃) δ ppm 1.26 (2H, t), 1.49-1.62 (2H, m), 1.95-2.04 (2H, m), 2.25-2.35 (2H, m), 2.36-2.42 (3H, m), 4.14-4.33 (2H, m), 4.53 (1H, d), 4.75 (1H, d), 6.24 (1H, dd), 7.60 (1H, dd), 7.81 (1H, d), 7.86 (1H, t), 8.22 (1H, dd).

### Intermediate 33

### 6′-(((1S,3S)-3-Aminocyclopentyl)amino)-2-oxo-2H-[1,3′-bipyridine]-5-carbonitrile

### Step A. i-33a

### tert-Butyl ((1S,3S)-3-((5-cyano-2-oxo-2H-[1,3′-bipyridin]-6′-yl)amino)cyclopentyl)carbamate

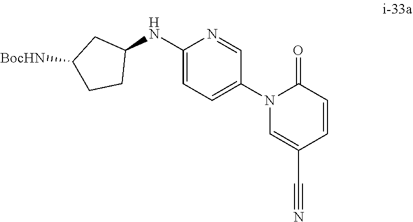 [SMILES: CC(C)(C)OC(=O)N[C@H]1CC[C@H](Nc2ccc(-n3cc(C#N)ccc3=O)cn2)C1]

According to GM2 tert-butyl ((1S,3S)-3-aminocyclopentyl)carbamate (425 mg, 2.12 mmol), 6′-chloro-2-oxo-2H-[1,3′-bipyridine]-5-carbonitrile (Intermediate 8) compound i-8d (410 mg, 1.77 mmol), Cs₂CO₃ (1.73 g, 5.31 mmol) and Pd-PEPPSI-IpentCl 2-methylpyridine (74 mg, 0.09 mmol) were reacted in 1,4-dioxane (10 mL) at 100° C. for 16 h to give upon non-aqueous work-up and purification by preparative TLC (MeOH:DCM=1:15) the title compound (390 mg, 56%) as a yellow solid.

### Step B. i-33b

### 6′-(((1S,3S)-3-Aminocyclopentyl)amino)-2-oxo-2H-[1,3′-bipyridine]-5-carbonitrile

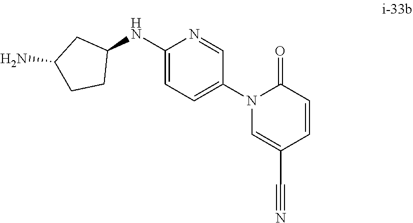 [SMILES: N#Cc1ccc(=O)n(-c2ccc(N[C@H]3CC[C@H](N)C3)nc2)c1]

According to GM6A tert-butyl ((1S,3S)-3-((5-cyano-2-oxo-2H-[1,3′-bipyridin]-6′-yl)amino)cyclopentyl)carbamate compound i-33a (370 mg, 0.94 mmol) was reacted with HCl in dioxane (4 M, 8 mL, 16 mmol) at 60° C. for 15 h to afford an unspecified HCl salt of the title compound (310 mg, 100%) as a brown solid.

### Intermediate 34

### i-34a

### 6-Chloro-1-methyl-1H-pyrazolo[3,4-b]pyrazine

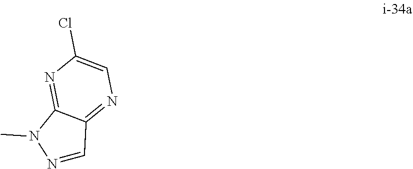

6-Chloro-1H-pyrazolo[3,4-b]pyrazine (CAS Reg. No. 1260664-81-0) (500 mg, 3.24 mmol) was added to K₃PO₄ (2060 mg, 9.71 mmol) and iodomethane (1378 mg, 9.71 mmol) in acetonitrile (20 mL). The resulting mixture was stirred at 60° C. for 3 h under nitrogen. The reaction mixture was quenched with saturated brine (100 mL), extracted with EtOAc (3×100 mL), the organic layer was dried over Na₂SO₄, filtered and evaporated. The residue was purified by preparative TLC (EtOAc:PE=1:1) to afford the title compound (209 mg, 38%) as a yellow solid. MS (ESI): m/z [M+H]⁺ 169. ¹H NMR (300 MHz, DMSO-d₆) δ ppm 4.06 (3H, s), 8.51 (1H, s), 8.71 (1H, s).

### Intermediate 35

### Step A. i-35a

### tert-Butyl ((1S,3S)-3-((3-cyano-2-oxo-2H-[1,3′-bipyridin]-6′-yl)amino)cyclopentyl)carbamate

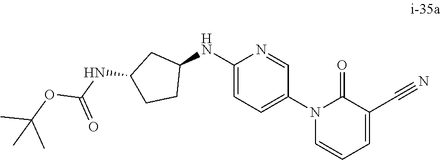 [SMILES: CC(C)(C)OC(=O)N[C@H]1CC[C@H](Nc2ccc(-n3cccc(C#N)c3=O)cn2)C1]

In a slight variation of GM2 6′-chloro-2-oxo-2H-[1,3′-bipyridine]-3-carbonitrile compound i-10a (560 mg, 1.89 mmol), tert-butyl ((1S,3S)-3-aminocyclopentyl)carbamate (CAS Reg. No. 645400-44-8) (1.13 g, 5.66 mmol), Cs₂CO₃ (1.84 g, 5.66 mmol) and Pd-PEPPSI-IpentCl 2-methylpyridine (79 mg, 0.09 mmol) were reacted in DMF (30 mL) at 100° C. for 15 h. The reaction mixture was filtered through a pad of Celite, the filter cake washed with DCM (3×10 mL) and the combined filtrates concentrated under reduced pressure. The obtained material was purified by C18-flash chromatography (gradient: 10-100% MeCN in water+0.1% NH₃ (aq), 910 mg material isolated) followed by preparative TLC (EtOAc:PE=3:1) to give the title compound (174 mg, 23%) as a grey solid. MS (ESI): m/z [M+H]⁺ 396.20.

### Step B. i-35b

### 6′-(((1S,3S)-3-Aminocyclopentyl)amino)-2-oxo-2H-[1,3′-bipyridine]-3-carbonitrile×4 HCl

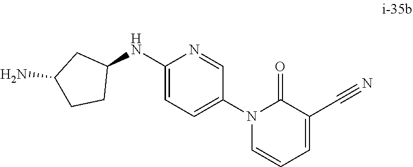 [SMILES: N#Cc1cccn(-c2ccc(N[C@H]3CC[C@H](N)C3)nc2)c1=O]

According to GM6A tert-butyl ((1S,3S)-3-((3-cyano-2-oxo-2H-[1,3′-bipyridin]-6′-yl)amino)cyclopentyl)carbamate compound i-35a (865 mg, 2.19 mmol) was reacted with HCl in methanol (4 M 8.0 mL, 32 mmol) in MeOH (20 mL) at 80° C. for 2 h to give the title compound (966 mg, 100%) as a brown gum. MS (ESI): m/z [M+H]⁺ 296.00.

### Intermediate 40

### Step A. i-40a

### tert-Butyl ((1S,3S)-3-((5-(1-methyl-2-oxo-1,2-dihydro-3H-imidazo[4,5-b]pyridin-3-yl)pyridin-2-yl)amino)cyclopentyl)carbamate

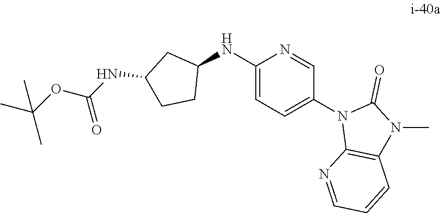 [SMILES: Cn1c(=O)n(-c2ccc(N[C@H]3CC[C@H](NC(=O)OC(C)(C)C)C3)nc2)c2ncccc21]

3-(6-Chloropyridin-3-yl)-1-methyl-1,3-dihydro-2H-imidazo[4,5-b]pyridin-2-one compound i-24a (200 mg, 0.77 mmol) was added to tert-butyl ((1S,3S)-3-aminocyclopentyl)carbamate (307 mg, 1.53 mmol), Cs₂CO₃ (750 mg, 2.30 mmol) and Pd-PEPPSI-IpentCl 2-methylpyridine (32 mg, 0.04 mmol) in 1,4-dioxane (10 mL) at 30° C. The resulting mixture was stirred at 100° C. for 15 h under a nitrogen atmosphere. The reaction mixture was diluted with water (50 mL) and the aqueous layer was extracted with EtOAc (8×30 mL). The organic layer was dried over Na₂SO₄, filtered and evaporated. The residue was purified by preparative TLC (EtOAc) to give the title compound (233 mg, 71%) as a yellow solid; MS (ESI) m/z [M+H]⁺ 425.1.

### Step B. i-40b

### 3-(6-(((1S,3S)-3-Aminocyclopentyl)amino)pyridin-3-yl)-1-methyl-1,3-dihydro-2H-imidazo[4,5-b]pyridin-2-one

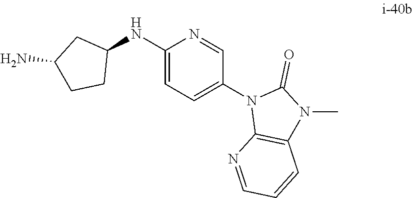 [SMILES: Cn1c(=O)n(-c2ccc(N[C@H]3CC[C@H](N)C3)nc2)c2ncccc21]

TFA (2 mL, 25.96 mmol) was added to tert-butyl ((1S,3S)-3-((5-(1-methyl-2-oxo-1,2-dihydro-3H-imidazo[4,5-b]pyridin-3-yl)pyridin-2-yl)amino)cyclopentyl)carbamate compound i-41a (220 mg, 0.52 mmol) in DCM (8 mL) at 30° C. The resulting mixture was stirred at 30° C. for 2 h. The solvent was removed under reduced pressure to give the TFA salt of the crude title compound (347 mg, 100%) as a brown gum; MS (ESI) m/z [M+H]⁺ 325.0.

### Intermediate 41

### Step A. i-41a

### tert-butyl ((1S,3S)-3-((5-(3-methyl-2,5-dioxoimidazolidin-1-yl)pyridin-2-yl)amino)cyclopentyl)carbamate

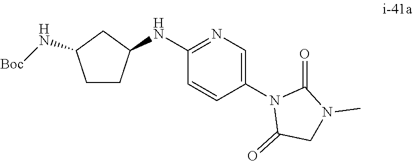 [SMILES: CN1CC(=O)N(c2ccc(N[C@H]3CC[C@H](NC(=O)OC(C)(C)C)C3)nc2)C1=O]

Cs₂CO₃ (3.70 g, 11.4 mmol) was added to tert-butyl ((1S,3S)-3-aminocyclopentyl)carbamate (CAS Reg. No. 645400-44-8) (1.3 g, 6.49 mmol), 3-(6-chloropyridin-3-yl)-1-methylimidazolidine-2,4-dione compound i-7b (1.32 g, 5.84 mmol) and Pd-PEPPSI-IpentCl 2-methylpyridine (CAS Reg. No. 1612891-29-8) (0.142 g, 0.17 mmol) in 1,4-dioxane (30 mL) at rt and the resulting suspension was stirred at 100° C. for 16 h under nitrogen. The reaction mixture was poured into saturated brine (300 mL), extracted with EtOAc (3×200 mL), the organic layer was dried (Na₂SO₄), filtered and evaporated to afford a pale yellow gum. The crude gum was triturated with EtOAc (20 mL) to give a solid which was collected by filtration and dried under vacuum (yellow solid). This solid was further purified by preparative TLC (EtOAc:PE=100:1) to afford (0.25 g, 10%) of the title compound as a white solid. MS (ESI): m/z [M+H]⁺ 390.

### Step B. i-41b

### 3-(6-(((1S,3S)-3-Aminocyclopentyl)amino)pyridin-3-yl)-1-methylimidazolidine-2,4-dione×2 HCl

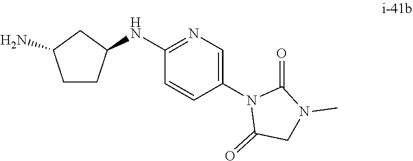 [SMILES: CN1CC(=O)N(c2ccc(N[C@H]3CC[C@H](N)C3)nc2)C1=O]

HCl in MeOH (4M) (5 mL, 20 mmol) was added to tert-butyl ((1S,3S)-3-((5-(3-methyl-2,5-dioxoimidazolidin-1-yl)pyridin-2-yl)amino)cyclopentyl)carbamate compound i-41a (250 mg, 0.64 mmol) in MeOH (15 mL) at rt and the resulting solution was stirred at 60° C. for 16 h. The solvent was removed under reduced pressure to give (220 mg, 95%) of the title compound as a pale yellow oil which solidified on standing. The product was used in the next step directly without further purification. MS (ESI): m/z [M+H]⁺ 290.

### Intermediate 42

### Step A. i-42a

### tert-Butyl ((1S,3S)-3-((5-methoxy-2-oxo-2H-[1,3′-bipyridin]-6′-yl)amino)cyclopentyl)carbamate

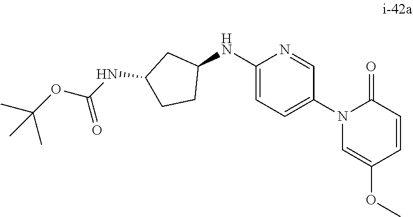 [SMILES: COc1ccc(=O)n(-c2ccc(N[C@H]3CC[C@H](NC(=O)OC(C)(C)C)C3)nc2)c1]

rel-(1R,2R)—N¹,N²-Dimethylcyclohexane-1,2-diamine (53 mg, 0.37 mmol) was added to tert-butyl ((1S,3S)-3-((5-iodopyridin-2-yl)amino)cyclopentyl)carbamate compound i-1a (300 mg, 0.74 mmol), 5-methoxypyridin-2-ol (233 mg, 1.86 mmol), Cs₂CO₃ (727 mg, 2.23 mmol) and Cu(I)I (70.8 mg, 0.37 mmol) in 1,4-dioxane (15 mL) at 25° C. The resulting suspension was stirred at 100° C. for 18 h under a nitrogen atmosphere. The reaction mixture was diluted with EtOAc (50 mL) and washed sequentially with water (3×75 mL). The organic layer was dried over Na₂SO₄, filtered and evaporated. The residue was purified by preparative TLC (EtOAc) to give the title compound (263 mg, 88%) as a brown solid. MS (ESI) m/z [M+H]⁺ 401.0.

### Step B. i-42b

### 6′-(((1S,3S)-3-Aminocyclopentyl)amino)-5-methoxy-2H-[1,3′-bipyridin]-2-one

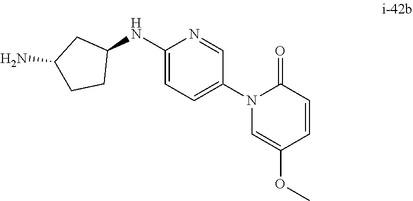 [SMILES: COc1ccc(=O)n(-c2ccc(N[C@H]3CC[C@H](N)C3)nc2)c1]

TFA (5 ml, 64.90 mmol) was added to tert-butyl ((1S,3S)-3-((5-methoxy-2-oxo-2H-[1,3′-bipyridin]-6′-yl)amino)cyclopentyl)carbamate compound i-42a (400 mg, 1.00 mmol) in DCM (15 mL) at 25° C. The resulting suspension was stirred at 25° C. for 18 h. The solvent was removed under reduced pressure to give the TFA salt of the crude title compound (1.1 g, 100%) as a black gum. MS (ESI) m/z [M+H]⁺ 301.0.

### Intermediate 43

### Step A. i-43a

### tert-Butyl ((1S,3S)-3-((3-chloro-2-oxo-2H-[1,3′-bipyridin]-6′-yl)amino)cyclopentyl)carbamate

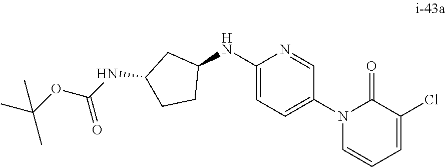 [SMILES: CC(C)(C)OC(=O)N[C@H]1CC[C@H](Nc2ccc(-n3cccc(Cl)c3=O)cn2)C1]

tert-Butyl ((1S,3S)-3-aminocyclopentyl)carbamate (467 mg, 2.33 mmol) was added to 3-chloro-6′-fluoro-2H-[1,3′-bipyridin]-2-one compound i-13a (262 mg, 1.17 mmol) and Na₂CO₃ (371 mg, 3.50 mmol) in DMSO (20 mL) at 25° C. The resulting mixture was stirred at 120° C. for 15 h. The reaction mixture was concentrated and diluted with EtOAc (250 mL) and washed with sat brine (4×50 mL). The organic layer was dried over Na₂SO₄, filtered and evaporated. The residue was purified by preparative TLC (EtOAc:PE=2:1) to give the title compound (456 mg, 97%) as a white solid. MS (ESI) m/z [M+H]⁺ 404.9.

### Step B. i-43b

### 6′-(((1S,3S)-3-Aminocyclopentyl)amino)-3-chloro-2H-[1,3′-bipyridin]-2-one

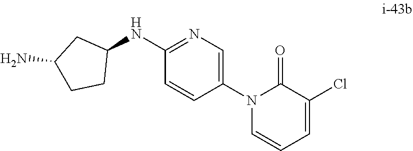 [SMILES: N[C@H]1CC[C@H](Nc2ccc(-n3cccc(Cl)c3=O)cn2)C1]

4 M HCl in dioxane (2 mL, 8.00 mmol) was added to tert-butyl ((1S,3S)-3-((3-chloro-2-oxo-2H-[1,3′-bipyridin]-6′-yl)amino)cyclopentyl)carbamate compound i-43a (425 mg, 1.05 mmol) in MeOH (20 mL) at 30° C. The resulting solution was stirred at 30° C. for 2 h. The solvent was removed under reduced pressure to give the HCl salt of the crude title compound (329 mg, 83%) as a yellow oil. MS (ESI) m/z [M+H]⁺ 305.0.

### Intermediate 44

### Step A. i-44a

### tert-Butyl ((1S,3S)-3-((5-chloro-2-oxo-2H-[1,3′-bipyridin]-6′-yl)amino)cyclopentyl)carbamate

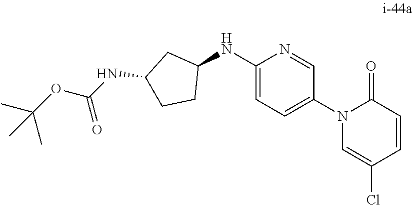 [SMILES: CC(C)(C)OC(=O)N[C@H]1CC[C@H](Nc2ccc(-n3cc(Cl)ccc3=O)cn2)C1]

rel-(1R,2R)—N¹,N²-Dimethylcyclohexane-1,2-diamine (106 mg, 0.74 mmol) was added to tert-butyl ((1S,3S)-3-((5-iodopyridin-2-yl)amino)cyclopentyl)carbamate compound i-1a (300 mg, 0.74 mmol), 5-chloropyridin-2 (1H)-one (193 mg, 1.49 mmol), Cu(I)I (142 mg, 0.74 mmol) and Cs₂CO₃ (727 mg, 2.23 mmol) in 1,4-dioxane (25 mL) at 26° C. The resulting solution was stirred at 100° C. for 18 h. The reaction mixture was diluted with EtOAc (75 mL) and washed sequentially with water (3×25 mL) and sat brine (3×20 mL). The organic layer was dried over Na₂SO₄, filtered and evaporated. The residue was purified by preparative TLC (EtOAc:PE=2:1) to give the title compound (298 mg, 99%) as a green solid. MS (ESI) m/z [M+H]⁺ 405.0.

### Step B. i-44b

### 6′-(((1S,3S)-3-Aminocyclopentyl)amino)-5-chloro-2H-[1,3′-bipyridin]-2-one

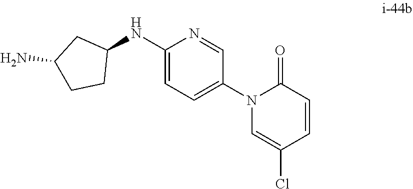 [SMILES: N[C@H]1CC[C@H](Nc2ccc(-n3cc(Cl)ccc3=O)cn2)C1]

TFA (5 mL, 64.90 mmol) was added to tert-butyl ((1S,3S)-3-((5-chloro-2-oxo-2H-[1,3′-bipyridin]-6′-yl)amino)cyclopentyl)carbamate compound i-44a (220 mg, 0.54 mmol) in DCM (20 mL) at 25° C. The resulting solution was stirred at 100° C. for 18 h. The solvent was removed under reduced pressure to give the TFA salt of the crude title compound (432 mg, 91%) as a brown gum. MS (ESI) m/z [M+H]⁺ 304.9.

### Intermediate 45

### Step A. i-45a

### tert-Butyl ((1S,3S)-3-((5-cyclopropylpyrazin-2-yl)amino)cyclopentyl)carbamate

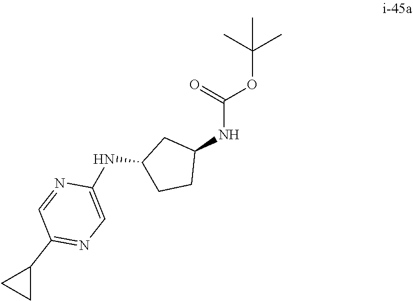 [SMILES: CC(C)(C)OC(=O)N[C@H]1CC[C@H](Nc2cnc(C3CC3)cn2)C1]

2-Bromo-5-cyclopropylpyrazine (CAS Reg. No. 1086382-78-6) (150 mg, 0.75 mmol) was added to Pd-PEPPSI-IpentCl 2-methylpyridine (CAS Reg. No. 1612891-29-8) (32 mg, 0.04 mmol), tert-butyl ((1S,3S)-3-aminocyclopentyl)carbamate (CAS Reg. No. 645400-44-8) (151 mg, 0.75 mmol) and Cs₂CO₃ (737 mg, 2.26 mmol) in 1,4-dioxane (10 mL) and the resulting mixture was stirred at 100° C. for 15 h under nitrogen. The reaction mixture was filtered through celite. The filter cake was washed with EtOAc (3×20 mL) and the filtrate was concentrated under reduced pressure. The obtained crude product was purified by preparative TLC (EtOAc:PE=2:1) to afford (180 mg, 75%) of the title compound as a yellow solid. MS (ESI): m/z [M+H]⁺ 319.1

### Step B. i-45b

### (1S,3S)—N¹-(5-cyclopropylpyrazin-2-yl)cyclopentane-1,3-diamine×3 TFA

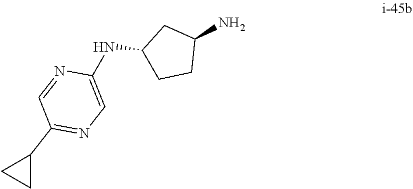 [SMILES: N[C@H]1CC[C@H](Nc2cnc(C3CC3)cn2)C1]

tert-Butyl ((1S,3S)-3-((5-cyclopropylpyrazin-2-yl)amino)cyclopentyl)carbamate compound i-45a (160 mg, 0.50 mmol) was added to a mixture of TFA (2 mL) and DCM (6 mL). The resulting mixture was stirred at rt for 3 h. The organic solvents were removed by evaporation to afford (267 mg, 95%) of the title compound as a brown oil which was directly used in the next step without further purification. MS (ESI): m/z [M+H]⁺ 219.3.

### Intermediate 46

### Step A. i-46a

### tert-Butyl ((1S,3S)-3-((5-(trifluoromethoxy)pyrazin-2-yl)amino)cyclopentyl)carbamate

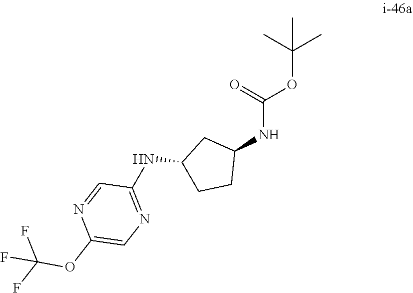 [SMILES: CC(C)(C)OC(=O)N[C@H]1CC[C@H](Nc2cnc(OC(F)(F)F)cn2)C1]

A mixture of 2-chloro-5-(trifluoromethoxy)pyrazine (CAS Reg. No. 1803596-66-8) (46 mg, 0.23 mmol), tert-butyl ((1S,3S)-3-aminocyclopentyl)carbamate (CAS Reg. No. 645400-44-8) (93 mg, 0.46 mmol), (oxybis(2,1-phenylene))bis(diphenylphosphane) (12 mg, 0.02 mmol), Pd₂dba₃ (21 mg, 0.02 mmol) and Cs₂CO₃ (0.226 g, 0.70 mmol) in 1,4-dioxane (2 mL) was stirred at 100° C. for 2 h. The mixture was diluted with DCM, water was added and the phases separated on a phase separator. The organic layer was concentrated and the residue combined with another batch (scale 0.09 mmol) and purified by flash chromatography on silica (gradient: 0-100% EtOAc in n-heptane) to give (66 mg, 57%) of the title compound as a brown syrup. MS (ESI): m/z [M+H]⁺ 363.2.

### Step B. i-46b

### (1S,3S)—N¹-(5-(trifluoromethoxy)pyrazin-2-yl)cyclopentane-1,3-diamine×TFA

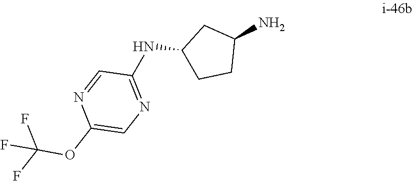 [SMILES: N[C@H]1CC[C@H](Nc2cnc(OC(F)(F)F)cn2)C1]

A mixture of tert-butyl ((1S,3S)-3-((5-(trifluoromethoxy)pyrazin-2-yl)amino)cyclo-pentyl)carbamate compound i-46a (42 mg, 0.12 mmol) in DCM (1.5 mL) and TFA (0.5 mL) was stirred at rt for 2 h. The mixture was concentrated under reduced pressure and directly used in next step without further purification (assuming quantitative yield). MS (ESI): m/z [M+H]⁺ 263.2.

### Intermediate 49

### Step A. i-49a

### 6′-Chloro-5-(1-(4-methoxybenzyl)-1H-1,2,3-triazol-4-yl)-2H-[1,3′-bipyridin]-2-one

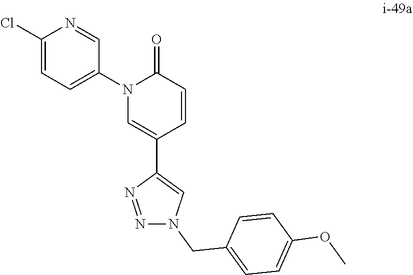 [SMILES: COc1ccc(Cn2cc(-c3ccc(=O)n(-c4ccc(Cl)nc4)c3)nn2)cc1]

Cu(I)I (103 mg, 1.04 mmol) was added to 6′-chloro-5-ethynyl-2H-[1,3′-bipyridin]-2-one compound i-63b (240 mg, 1.04 mmol) and 1-(azidomethyl)-4-methoxybenzene (255 mg, 1.56 mmol) in 1,4-dioxane (10 mL) at rt and the resulting suspension was stirred at 100° C. for 16 h under nitrogen. The mixture was filtered through a Celite pad and the filter cake washed with EtOAc (20 mL). The filtrate was concentrated under reduced pressure and the residue purified by preparative TLC (MeOH:DCM=1:20) to afford (410 mg, 100%) of the title compound as a white solid. MS (ESI): m/z [M+H]⁺ 394.0.

### Step D. i-49d

### 5-(1-(4-Methoxybenzyl)-1H-1,2,3-triazol-4-yl)-6′-(((1S,3S)-3-((5-methylpyrazin-2-yl)amino)cyclopentyl)amino)-2H-[1,3′-bipyridin]-2-one

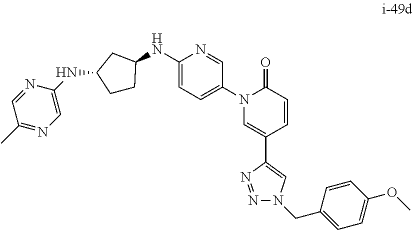

Cs₂CO₃ (207 mg, 0.63 mmol) was added to a mixture of 6′-chloro-5-(1-(4-methoxybenzyl)-1H-1,2,3-triazol-4-yl)-2H-[1,3′-bipyridin]-2-one compound i-49a (50 mg, 0.13 mmol), the 2×HCl salt of (1S,3S)—N¹-(5-methylpyrazin-2-yl)cyclopentane-1,3-diamine compound i-31b (67 mg, 0.25 mmol) and Pd-PEPPSI-IpentCl 2-methylpyridine (CAS Reg. No. 1612891-29-8) (11 mg, 0.01 mmol) in 1,4-dioxane (3 mL) at rt and the resulting mixture was stirred at 100° C. for 16 h under nitrogen. The reaction mixture was poured into water (100 mL) and extracted with EtOAc (2×100 mL). The combined organic layers were dried over Na₂SO₄, filtered and evaporated and the residue purified by preparative TLC (NH₃ in MeOH (7 M):DCM=1:20) to give the title compound (12 mg, 17%) as a brown solid. MS (ESI): m/z [M+H]⁺ 550.0.

### Intermediate 50

### Step A. i-50a

### 5-(1-(4-Methoxybenzyl)-1H-pyrazol-4-yl)pyridin-2-ol

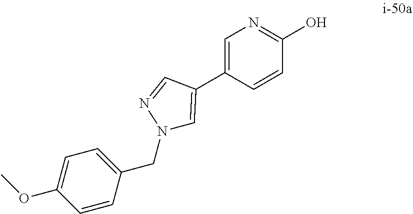 [SMILES: COc1ccc(Cn2cc(-c3ccc(O)nc3)cn2)cc1]

XPhos (0.431 g, 0.90 mmol), XPhos Pd G3 (CAS Reg. No. 1445085-55-1) (0.383 g, 0.45 mmol), 1-(4-methoxybenzyl)-4-(4,4,5,5-tetramethyl-1,3,2-dioxaborolan-2-yl)-1H-pyrazole (CAS Reg. No. 110539-88-0) (2.13 g, 6.79 mmol), 5-iodopyridin-2-ol (CAS Reg. No. 13472-79-2) (1.0 g, 4.5 mmol) and Cs₂CO₃ (4.42 g, 13.6 mmol) in dioxane (10 mL) and water (2 mL) were stirred under an atmosphere of nitrogen at 80° C. for 15 h. The mixture was filtered through a Celite pad. The filter cake was washed with EtOAc (20 mL) and the filtrate was concentrated under reduced pressure to give the crude product which was purified by flash C18-flash chromatography (elution gradient: 0-50% MeCN in water) to afford (160 mg, 13%) of the title compound as a white solid. MS (ESI): m/z [M+H]⁺ 282.0.

### Step B. i-50b

### 6′-Chloro-5-(1-(4-methoxybenzyl)-1H-pyrazol-4-yl)-2H-[1,3′-bipyridin]-2-one

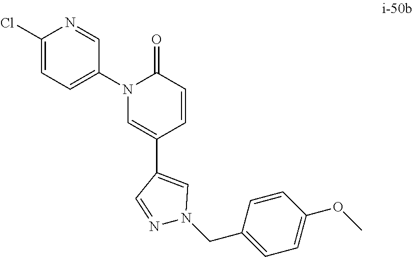 [SMILES: COc1ccc(Cn2cc(-c3ccc(=O)n(-c4ccc(Cl)nc4)c3)cn2)cc1]

Cu(I)I (102 mg, 0.53 mmol) was added to 5-(1-(4-methoxybenzyl)-1H-pyrazol-4-yl)pyridin-2-ol compound i-50a (150 mg, 0.53 mmol), 2-chloro-5-iodopyridine (CAS Reg. No. 69045-79-0) (128 mg, 0.53 mmol), rel-(1R,2R)—N¹,N²-dimethylcyclohexane-1,2-diamine (114 mg, 0.80 mmol) and Cs₂CO₃ (347 mg, 1.07 mmol) in 1,4-dioxane (10 mL) at rt and the resulting suspension was stirred at 60° C. for 5 h under nitrogen. The mixture was filtered through a Celite pad. The filter cake was washed with EtOAc (20 mL) and the filtrate concentrated under reduced pressure. The obtained residue was first purified by preparative TLC (MeOH:DCM=1:10) and then by flash C18-flash chromatography (elution gradient: 0-30% MeCN in water) to afford (150 mg, 72%) of the title compound as a white solid. MS (ESI): m/z [M+H]⁺ 393.0.

### Step E. i-50e

### 5-(1-(4-Methoxybenzyl)-1H-pyrazol-4-yl)-6′-(((1S,3S)-3-((5-methylpyrazin-2-yl)amino)cyclopentyl)amino)-2H-[1,3′-bipyridin]-2-one

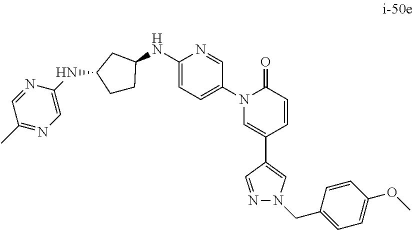

Cs₂CO₃ (622 mg, 1.91 mmol) was added to a mixture of 6′-chloro-5-(1-(4-methoxybenzyl)-1H-pyrazol-4-yl)-2H-[1,3′-bipyridin]-2-one compound i-50b (250 mg, 0.64 mmol), (1S,3S)—N¹-(5-methylpyrazin-2-yl)cyclopentane-1,3-diamine compound i-31b (245 mg, 1.27 mmol) and Pd-PEPPSI-IpentCl 2-methylpyridine (CAS Reg. No. 1612891-29-8) (54 mg, 0.06 mmol) in DMF (15 mL) at rt and the resulting mixture was stirred at 100° C. for 16 h under nitrogen. The reaction mixture was diluted with EtOAc (150 mL) and washed sequentially with sat. brine (7×200 mL). The organic layer was dried (Na₂SO₄), filtered and evaporated and the crude product was purified by preparative TLC (MeOH:DCM=1:15) to give the title compound (150 mg, 43%) as a brown solid. MS (ESI): m/z [M+H]⁺ 549.0.

### Intermediate 55

### i-55a

### 5-Bromo-2-(difluoromethoxy)-3-methylpyrazine

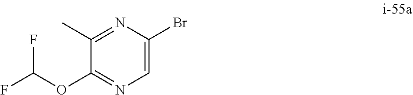

5-Bromo-3-methylpyrazin-2-ol (CAS Reg. No. 100047-56-1) (300 mg, 1.59 mmol) was added to KOH (5 mL, 30.0 mmol) and diethyl (bromodifluoromethyl)phosphonate (1271 mg, 4.76 mmol) in MeCN (5 mL) and the resulting mixture stirred at 20° C. for 1 day. The reaction mixture was quenched with water (100 mL) and extracted with EtOAc (3×75 mL). The combined organic layers were dried over Na₂SO₄, filtered and evaporated and the residue purified by preparative TLC (EtOAc:PE=1:5) to give the title compound (130 mg, 34%) as a white solid. 1H NMR (300 MHz, DMSO-d₆) δ ppm 2.46 (d, 3H), 7.63 (t, 1H), 8.39 (d, 1H).

### Intermediate 56

### i-56a

### 3-(Trifluoromethoxy)pyridin-2 (1H)-one

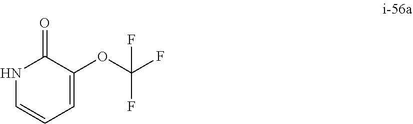 [SMILES: O=c1[nH]cccc1OC(F)(F)F]

TFA (5.0 mL, 65 mmol) was added to 2-((4-methoxybenzyl)oxy)-3-(trifluoromethoxy)pyridine compound i-9a (0.92 g, 3.1 mmol) in DCM (2 mL). The purple solution was stirred for 30 min at rt and then concentrated. The residue was co-evaporated with toluene (×3) and a small amount of DCM was added to the residue. A solid was formed that was filtered off and washed with a small amount of DCM and EtOAc. The solid was dried under vacuum to give the title compound (0.36 g, 65%) as a white solid. MS (ESI): m/z [M+H]⁺ 179.9.

### Intermediate 60

### Step A. i-60a

### 3-((6-Chloropyridin-3-yl)amino)-4-nitrobenzonitrile

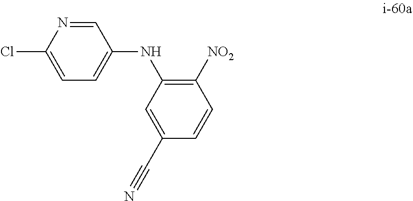 [SMILES: N#Cc1ccc([N+](=O)[O-])c(Nc2ccc(Cl)nc2)c1]

K₂CO₃ (6.45 g, 46.7 mmol) was added to 3-fluoro-4-nitrobenzonitrile (CAS Reg. No 218632-01-0) (3.10 g, 18.7 mmol), 6-chloropyridin-3-amine (CAS Reg. No. 5350-93-6) (2.0 g, 15.6 mmol) and Pd(OAc)₂ (0.262 g, 1.17 mmol) and XantPhos (1.35 g, 2.33 mmol) in MeCN (40 mL) at rt and the resulting mixture was stirred at 80° C. for 15 h under nitrogen. The reaction mixture was filtered through celite and the filter cake washed with DCM (3×50 mL). The combined filtrates were concentrated under reduced pressure and taken up with EtOAc (250 mL). The organic layer was washed sequentially with sat. NaHCO₃ (3×250 mL), dried (Na₂SO₄), filtered and evaporated. The obtained residue was purified by preparative TLC (DCM) to afford (0.63 g, 15%) of the title compound as a yellow solid. MS (ESI): m/z [M+H]⁺ 275.1.

### Step B. i-60b

### 4-Amino-3-((6-chloropyridin-3-yl)amino)benzonitrile

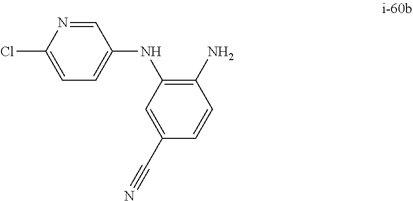 [SMILES: N#Cc1ccc(N)c(Nc2ccc(Cl)nc2)c1]

Zinc (971 mg, 14.9 mmol) was added to 3-((6-chloropyridin-3-yl)amino)-4-nitrobenzonitrile compound i-60a (510 mg, 1.86 mmol) and NH₄Cl (795 mg, 14.8 mmol) in EtOH (15 mL) at rt and the resulting mixture was stirred at 60° C. for 5 h. The reaction mixture was filtered through celite and the filter cake washed with EtOH (3×50 mL). The combined filtrates were concentrated under reduced pressure to afford (363 mg, 80%) of the title compound as a brown solid which was used in the next step directly without further purification. MS (ESI): m/z [M+H]⁺ 245.1.

### Step C. i-60c

### 3-(6-Chloropyridin-3-yl)-2-oxo-2,3-dihydro-1H-benzo[d]imidazole-5-carbonitrile

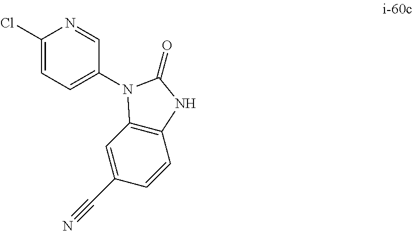 [SMILES: N#Cc1ccc2[nH]c(=O)n(-c3ccc(Cl)nc3)c2c1]

CDI (795 mg, 4.90 mmol) was added to 4-amino-3-((6-chloropyridin-3-yl)amino)benzonitrile compound i-60b (300 mg, 1.23 mmol) in DMF (2 mL) at rt and the resulting mixture was stirred at 80° C. for 24 h. The solid was filtered off to afford the crude product which was triturated with MeCN and dried under vacuum to give (242 mg, 73%) of the title compound as a pink solid. MS (ESI): m/z [M+H]⁺ 271.0.

### Step D. i-60d

### 3-(6-Chloropyridin-3-yl)-1-methyl-2-oxo-2,3-dihydro-1H-benzo[d]imidazole-5-carbonitrile

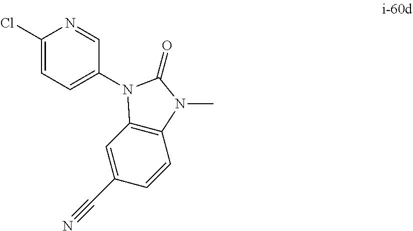 [SMILES: Cn1c(=O)n(-c2ccc(Cl)nc2)c2cc(C#N)ccc21]

Cs₂CO₃ (542 mg, 1.66 mmol) was added to a mixture of 3-(6-chloropyridin-3-yl)-2-oxo-2,3-dihydro-1H-benzo[d]imidazole-5-carbonitrile compound i-60c (150 mg, 0.55 mmol) and CH₃I (118 mg, 0.83 mmol) in DMF (5 mL) at rt and it was stirred at 100° C. for 18 h. The reaction mixture was diluted with EtOAc (50 mL) and washed sequentially with brine (3×50 mL). The organic layer was dried (Na₂SO₄), filtered and evaporated and the obtained material was triturated with MeCN. The formed solid was collected by filtration and dried under vacuum to give (110 mg, 70%) of the title compound as a pink solid. MS (ESI): m/z [M+H]⁺ 285.1.

### Intermediate 61

### Step A. i-61a

### 4-((6-Chloropyridin-3-yl)amino)-3-nitrobenzonitrile

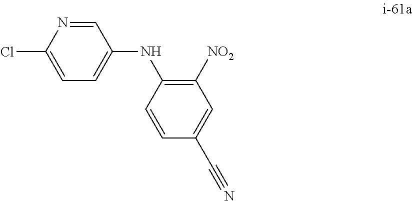 [SMILES: N#Cc1ccc(Nc2ccc(Cl)nc2)c([N+](=O)[O-])c1]

Pd(OAc)₂ (0.262 g, 1.17 mmol) was added to a mixture of K₂CO₃ (6.45 g, 46.7 mmol), 4-fluoro-3-nitrobenzonitrile (CAS Reg. No. 1009-35-4) (2.84 g, 17.1 mmol), 6-chloropyridin-3-amine CAS Reg. No. 5350-93-6 (2.0 g, 15.6 mmol) and XantPhos (1.35 g, 2.33 mmol) in MeCN (40 mL) at rt and it was stirred at 80° C. for 15 h under nitrogen. The reaction mixture was filtered through Celite and the filter cake washed with DCM (3×200 mL). The combined filtrates were concentrated under reduced pressure and taken up with EtOAc (300 mL). The organic layer was washed sequentially with sat. NaHCO₃ (3×400 mL), dried over (Na₂SO₄), filtered and evaporated. The obtained material was purified by preparative TLC (DCM) to afford (0.70 g, 16%) of the title compound as a yellow solid. MS (ESI): m/z [M+H]⁺ 275.0

### Step B. i-61b

### 3-Amino-4-((6-chloropyridin-3-yl)amino)benzonitrile

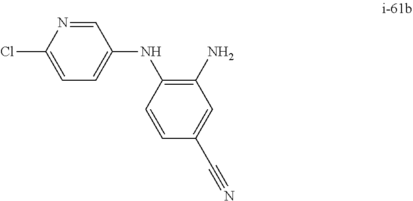 [SMILES: N#Cc1ccc(Nc2ccc(Cl)nc2)c(N)c1]

Zinc (1.33 g, 20.4 mmol) was added to NH₄Cl (1.09 g, 20.4 mmol), 4-((6-chloropyridin-3-yl)amino)-3-nitrobenzonitrile compound i-61a (0.70 g, 2.54 mmol) in EtOH (15 mL) at rt and the resulting mixture was stirred at 60° C. for 5 h. The reaction mixture was filtered through Celite and the filter cake washed with EtOH (3×50 mL). The combined filtrates were concentrated under reduced pressure to afford (0.51 g, 81%) of the title compound as a brown solid which was used in the next step without further purification. MS (ESI): m/z [M+H]⁺ 245.1.

### Step C. i-61c

### 1-(6-Chloropyridin-3-yl)-2-oxo-2,3-dihydro-1H-benzo[d]imidazole-5-carbonitrile

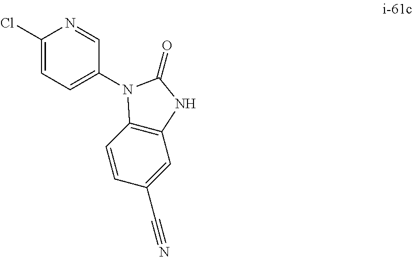 [SMILES: N#Cc1ccc2c(c1)[nH]c(=O)n2-c1ccc(Cl)nc1]

CDI (1.19 g, 7.36 mmol) was added to 3-amino-4-((6-chloropyridin-3-yl)amino)benzonitrile compound i-61b (450 mg, 1.84 mmol) in DMF (10 mL) at rt and the resulting mixture was stirred at 80° C. for 24 h. The solid was filtered off to afford the crude product which was triturated with MeCN and dried under vacuum to give (342 mg, 69%) of the title compound as a pink solid. MS (ESI): m/z [M+H]⁺ 270.9.

### Step D. i-61d

### 1-(6-Chloropyridin-3-yl)-3-methyl-2-oxo-2,3-dihydro-1H-benzo[d]imidazole-5-carbonitrile

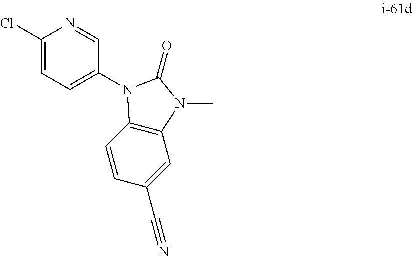 [SMILES: Cn1c(=O)n(-c2ccc(Cl)nc2)c2ccc(C#N)cc21]

CH₃I (157 mg, 1.11 mmol) was added to a mixture of 1-(6-chloropyridin-3-yl)-2-oxo-2,3-dihydro-1H-benzo[d]imidazole-5-carbonitrile compound i-61c (200 mg, 0.74 mmol) and Cs₂CO₃ (722 mg, 2.22 mmol) in DMF (8 mL) at rt and it was stirred at 100° C. for 15 h. The reaction mixture was diluted with EtOAc (100 mL) and washed sequentially with brine (3×100 mL). The organic layer was dried (Na₂SO₄), filtered and evaporated to afford (119 mg, 57%) of the title compound as a yellow solid which was used in the next step without further purification. MS (ESI): m/z [M+H]⁺ 285.0.

### Intermediate 63

### Step A. i-63a

### 5-((tert-Butyldimethylsilyl)ethynyl)-6′-chloro-2H-[1,3′-bipyridin]-2-one

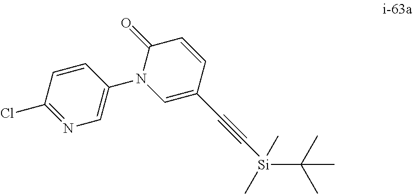 [SMILES: CC(C)(C)[Si](C)(C)C#Cc1ccc(=O)n(-c2ccc(Cl)nc2)c1]

Cs₂CO₃ (2.23 g, 6.86 mmol) was added to 5-((tert-butyldimethylsilyl)ethynyl)pyridin-2 (1H)-one (CAS Reg. No. 2448766-74-1) (800 mg, 3.43 mmol), 2-chloro-5-iodopyridine (CAS Reg. No. 69045-79-0) (821 mg, 3.43 mmol), Cu(I)I (653 mg, 3.43 mmol) and rel-(1R,2R)—N¹,N²-dimethylcyclohexane-1,2-diamine (731 mg, 5.14 mmol) in 1,4-dioxane (20 mL) at rt and the resulting suspension was stirred at 60° C. for 5 h under nitrogen. The mixture was combined with a second batch made in the same way and filtered through a Celite pad. The filter cake was washed with EtOAc (100 mL) and the combined filtrates were concentrated under reduced pressure. The crude product was purified by flash chromatography on silica (gradient: 0-50% EtOAc in n-heptane) to afford (590 mg, 25%) of the title compound as a white solid. MS (ESI): m/z [M+H]⁺ 345.0.

### Step B. i-63b

### 6′-Chloro-5-ethynyl-2H-[1,3′-bipyridin]-2-one

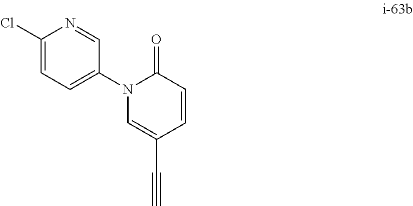 [SMILES: C#Cc1ccc(=O)n(-c2ccc(Cl)nc2)c1]

A solution of TBAF in THE (1 M, 8.55 mL, 8.55 mmol) was added to 5-((tert-butyldimethylsilyl)ethynyl)-6′-chloro-2H-[1,3′-bipyridin]-2-one compound i-63a (590 mg, 1.71 mmol) in THE (15 mL) at rt and the resulting solution was stirred at for 2 h. The solvent was removed under reduced pressure and the residue was purified by preparative TLC (EtOAc:PE=2:1) followed by flash C18-flash chromatography (elution gradient: 0-30% MeCN in water) to afford (290 mg, 74%) of the title compound as a beige solid. MS (ESI): m/z [M+H]⁺ 231.0.

### Intermediate 65

### Step A. i-65a

### 6′-Chloro-5-(1H-tetrazol-5-yl)-2H-[1,3′-bipyridin]-2-one

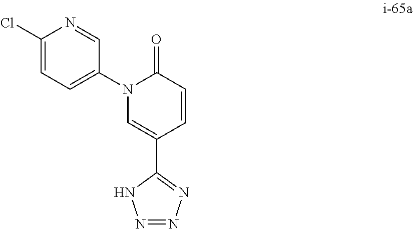 [SMILES: O=c1ccc(-c2nnn[nH]2)cn1-c1ccc(Cl)nc1]

Bis(4-nitrophenyl) phosphorazidate (CAS Reg. No. 51250-91-0) (880 mg, 2.4 mmol) was added to a solution of 6′-chloro-2-oxo-2H-[1,3′-bipyridine]-5-carboxamide compound i-8c (300 mg, 1.20 mmol) in pyridine (5 mL) under air. The resulting mixture was stirred at 90° C. for 16 h, subsequently cooled to rt and concentrated under reduced pressure. The obtained residue was purified by C18-flash chromatography (gradient: 0-20% MeCN in water) to give the title compound (210 mg, 64%) as a white solid. MS (ESI): m/z [M+H]⁺ 275.0.

### Step B. i-65b

### 6′-Chloro-5-(1-(4-methoxybenzyl)-1H-tetrazol-5-yl)-2H-[1,3′-bipyridin]-2-one

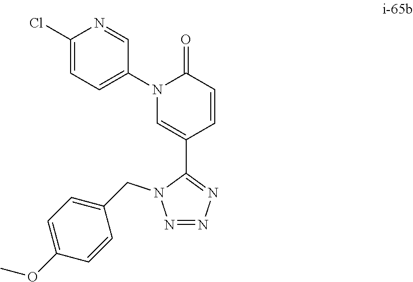 [SMILES: COc1ccc(Cn2nnnc2-c2ccc(=O)n(-c3ccc(Cl)nc3)c2)cc1]

4-Methoxybenzyl chloride (CAS Reg. No. 824-94-2) (690 mg, 4.4 mmol) was added to a mixture of 6′-chloro-5-(1H-tetrazol-5-yl)-2H-[1,3′-bipyridin]-2-one compound i-65a (810 mg, 2.95 mmol) and K₂CO₃ (815 mg, 5.90 mmol) in DMF (15 mL) and the resulting suspension was stirred at 80° C. for 3 h. The reaction mixture was diluted with water (100 mL) and extracted with EtOAc (3×150 mL). The combined organic layers were dried over Na₂SO₄, filtered and evaporated and the crude material was purified by C18-flash chromatography (gradient: 30-40% MeCN in water) to give the title compound (1000 mg, 86%) as a white solid. MS (ESI): m/z [M+H]⁺ 395.1.

### Intermediate 67

### Step A. i-67a

### 5-(1-Methyl-1H-pyrazol-4-yl)pyridin-2-ol

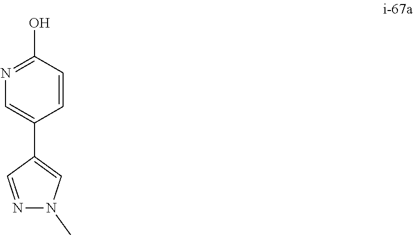

A mixture of 5-iodo-2 (1H)-pyridinone (CAS Reg. No. 13472-79-2) (500 mg, 2.26 mmol), 1-methyl-4-(4,4,5,5-tetramethyl-1,3,2-dioxaborolan-2-yl)-1H-pyrazole (CAS Reg. No. 761446-44-0) (565 mg, 2.71 mmol) and XPhos (CAS Reg. No. 564483-18-7) (108 mg, 0.23 mmol) in 1,4-dioxane (10 mL) and water (1 mL) was treated with XPhos Pd G3 (CAS Reg. No. 1445085-55-1) (192 mg, 0.23 mmol) under nitrogen and stirred at 100° C. for 18 h. The reaction mixture was cooled to rt and filtered through a pad of Celite. The filter cake was washed with EtOAc (100 mL) and the combined filtrates concentrated under reduced pressure. The obtained material was purified by flash chromatography on silica (gradient: 10-20% MeOH in DCM) to afford the title compound (250 mg, 63%) as a green solid. MS (ESI): m/z [M+H]⁺ 175.9.

### Step B. i-67b

### 6′-Chloro-5-(1-methyl-1H-pyrazol-4-yl)-2H-[1,3′-bipyridin]-2-one

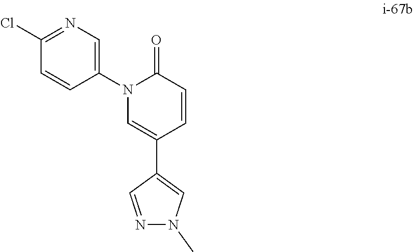 [SMILES: Cn1cc(-c2ccc(=O)n(-c3ccc(Cl)nc3)c2)cn1]

According to GM3 5-(1-methyl-1H-pyrazol-4-yl)pyridin-2-ol compound i-67a (250 mg, 1.43 mmol), 2-chloro-5-iodopyridine (342 mg, 1.43 mmol), rel-(1R,2R)—N¹,N¹-dimethylcyclo-hexane-1,2-diamine (203 mg, 1.43 mmol), Cs₂CO₃ (1.4 g, 4.3 mmol) and Cu(I)I (272 mg, 1.43 mmol) were reacted in 1,4-dioxane (8 mL) at 100° C. for 18 h to give upon non-aqueous work-up (EtOAc) and flash chromatography on silica (gradient: 0-10% MeOH in DCM) the title compound (250 mg, 61%) as a green solid. m/z [M+H]⁺ 287.1/289 (Cl isotope pattern).

### Intermediate 68b

### i-68b

### 6′-(((1S,3S)-3-((5-Methylpyrazin-2-yl)amino)cyclopentyl)amino)-3-(2-(tetrahydro-2H-pyran-2-yl)-2H-1,2,3-triazol-4-yl)-2H-[1,3′-bipyridin]-2-one

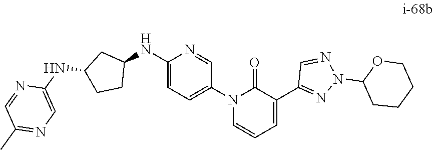

3-Chloro-6′-(((1S,3S)-3-((5-methylpyrazin-2-yl)amino)cyclopentyl)amino)-2H-[1,3′-bipyridin]-2-one Example 47 (150 mg, 0.38 mmol) was added to a mixture of 1-(tetrahydro-2H-pyran-2-yl)-5-(4,4,5,5-tetramethyl-1,3,2-dioxaborolan-2-yl)-1H--1,2,3-triazole (CAS Reg. No. 2710298-24-9) (211 mg, 0.76 mmol), Cs₂CO₃ (369 mg, 1.13 mmol), XPhos (CAS Reg. No. 564483-18-7) (18 mg, 0.04 mmol) and XPhos Pd G3 (CAS Reg. No. 1445085-55-1) (32 mg, 0.04 mmol) in 1,4-dioxane (10 mL) and water (2.5 mL) under nitrogen. The resulting reaction mixture was stirred at 100° C. for 15 h and subsequently poured into saturated brine (125 mL). It was extracted with EtOAc (4×100 mL) and the combined organic layers were dried over Na₂SO₄, filtered and evaporated. The crude material was purified by preparative TLC (7 M NH₃ in MeOH:DCM=1:15) to give the title compound (160 mg, 82%) as a yellow solid. MS (ESI): m/z [M+H]⁺ 514.1.

### Intermediate 69

### StepA. i-69a

### tert-Butyl ((1S,3S)-3-((5-(3-methyl-6-oxopyridazin-1 (6H)-yl)pyridin-2-yl)amino)cyclopentyl)carbamate

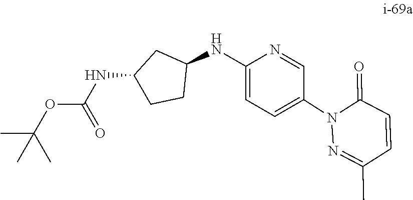 [SMILES: Cc1ccc(=O)n(-c2ccc(N[C@H]3CC[C@H](NC(=O)OC(C)(C)C)C3)nc2)n1]

rel-(1R,2R)—N¹,N²-Dimethylcyclohexane-1,2-diamine (70.5 mg, 0.50 mmol) was added to a mixture of tert-butyl ((1S,3S)-3-((5-iodopyridin-2-yl)amino)cyclopentyl)carbamate compound i-1a (400 mg, 0.99 mmol), 6-methylpyridazin-3 (2H)-one (CAS Reg. No. 13327-27-0) (218 mg, 1.98 mmol), Cu(I)I (94 mg, 0.50 mmol) and Cs₂CO₃ (970 mg, 2.98 mmol) in 1,4-dioxane (20 mL) at 20° C. and the resulting suspension was stirred at 100 C for 15 h under a nitrogen atmosphere. The reaction mixture was diluted with water (75 mL) and extracted with EtOAc (3×150 mL). The organic layers were combined, dried over Na₂SO₄, filtered and evaporated and the residue purified by preparative TLC (EtOAc) to give the title compound (369 mg, 97%) as a yellow solid. MS (ESI): m/z [M+H]⁺ 386.0.

### Step B. i-69b

### 2-(6-(((1S,3S)-3-Aminocyclopentyl)amino)pyridin-3-yl)-6-methylpyridazin-3 (2H)-one 3×HCl

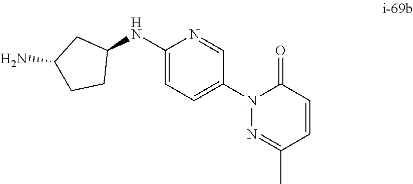 [SMILES: Cc1ccc(=O)n(-c2ccc(N[C@H]3CC[C@H](N)C3)nc2)n1]

A solution of HCl (4 M in MeOH, 5.0 mL, 20 mmol) was added to a stirred solution of tert-butyl ((1S,3S)-3-((5-(3-methyl-6-oxopyridazin-1 (6H)-yl)pyridin-2-yl)amino)cyclopentyl)carbamate compound i-69a (355 mg, 0.92 mmol) in MeOH (10 mL) at 20° C. and the resulting solution was stirred at 60° C. for 2 h. The solvent was removed under reduced pressure to give an unspecified HCl salt of the title compound (360 mg, 99%) as a pale-yellow solid which was directly used in the next step without further purification. MS (ESI): m/z [M+H]⁺ 285.9.

### Intermediate 70

### Step A. i-70a

### tert-Butyl ((1S,3S)-3-((5-(5-chloro-6-oxopyridazin-1 (6H)-yl)pyridin-2-yl)amino)cyclopentyl)carbamate

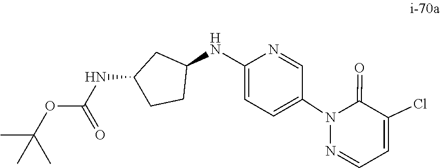 [SMILES: CC(C)(C)OC(=O)N[C@H]1CC[C@H](Nc2ccc(-n3nccc(Cl)c3=O)cn2)C1]

tert-Butyl ((1S,3S)-3-((5-iodopyridin-2-yl)amino)cyclopentyl)carbamate compound i-1a (400 mg, 0.99 mmol) was added to a mixture of 4-chloropyridazin-3 (2H)-one (CAS Reg. No. 1677-79-8) (518 mg, 3.97 mmol), Cu(I)I (94 mg, 0.50 mmol), Cs₂CO₃ (970 mg, 2.98 mmol) and rel-(1R,2R)—N¹,N²-dimethylcyclohexane-1,2-diamine (70.5 mg, 0.50 mmol) in dioxane (5 mL) at 25° C. under a nitrogen atmosphere and it was stirred at 100° C. for 18 h. The reaction mixture was concentrated, diluted with EtOAc (75 mL) and washed sequentially with NH₃ (aq) (3×50 mL). The organic layer was kept and the aqueous layers were combined and re-extracted with EtOAc (2×50 mL). The combined organic layers were dried over Na₂SO₄, filtered and evaporated and the residue was purified by preparative TLC (7 M NH₃ in MeOH:DCM=1:30) to give the title compound (90 mg, 22%) as a yellow solid. This procedure was repeated once more and the two batches were combined. MS (ESI): m/z [M+H]⁺ 406.0.

### Step B. i-70b

### 2-(6-(((1S,3S)-3-Aminocyclopentyl)amino)pyridin-3-yl)-4-chloropyridazin-3 (2H)-one

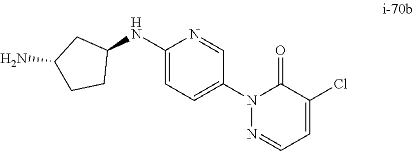 [SMILES: N[C@H]1CC[C@H](Nc2ccc(-n3nccc(Cl)c3=O)cn2)C1]

tert-Butyl ((1S,3S)-3-((5-(5-chloro-6-oxopyridazin-1 (6H)-yl)pyridin-2-yl)amino)cyclopentyl)carbamate compound i-70a (165 mg, 0.41 mmol) was added to a mixture of HCl (4 M in MeOH, 1.2 mL) and MeOH (4.8 mL) at 25° C. The resulting solution was stirred at 25° C. for 1 h and subsequently diluted with DCM (20 mL). The solvent was removed under reduced pressure to give an unspecified HCl salt of the title compound (123 mg, 99%) as a yellow solid that was directly used in the next step without further purification. MS (ESI): m/z [M+H]⁺ 305.9.

### Intermediate 71

### Step A. i-71a

### tert-Butyl ((1S,3S)-3-((5-(3-chloro-6-oxopyridazin-1 (6H)-yl)pyridin-2-yl)amino)cyclopentyl)carbamate

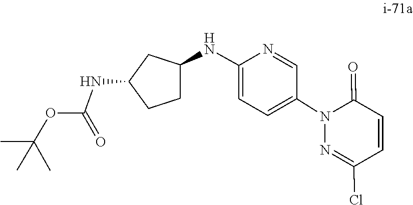 [SMILES: CC(C)(C)OC(=O)N[C@H]1CC[C@H](Nc2ccc(-n3nc(Cl)ccc3=O)cn2)C1]

rel-(1R,2R)—N¹,N²-Dimethylcyclohexane-1,2-diamine (141 mg, 0.99 mmol) was added to a mixture of tert-butyl ((1S,3S)-3-((5-iodopyridin-2-yl)amino)cyclopentyl)carbamate compound i-1a (400 mg, 0.99 mmol), Cu(I)I (189 mg, 0.99 mmol), 6-chloropyridazin-3 (2H)-one (CAS Reg. No. 19064-67-6) (324 mg, 2.48 mmol) and K₂CO₃ (411 mg, 2.98 mmol) in 1,4-dioxane (15 mL) at 25° C. and it was stirred at 100° C. for 18 h under a nitrogen atmosphere. The reaction mixture was concentrated, diluted with EtOAc (150 mL) and washed sequentially with sat. brine (3×50 mL). The organic layer was dried over Na₂SO₄, filtered and evaporated and the residue purified by preparative TLC (MeOH:DCM=1:20) to give the title compound (180 mg, 44%) as a yellow solid. MS (ESI): m/z [M+H]⁺ 405.9.

### Step B. i-71b

### 2-(6-(((1S,3S)-3-Aminocyclopentyl)amino)pyridin-3-yl)-6-chloropyridazin-3 (2H)-one HCl

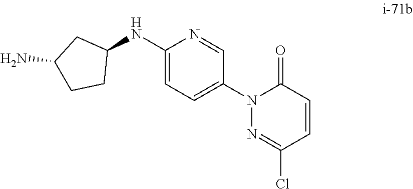 [SMILES: N[C@H]1CC[C@H](Nc2ccc(-n3nc(Cl)ccc3=O)cn2)C1]

A solution of HCl (4 M in 1,4-dioxane, 2 mL, 8 mmol) was added to a mixture of tert-butyl ((1S,3S)-3-((5-(3-chloro-6-oxopyridazin-1 (6H)-yl)pyridin-2-yl)amino)cyclopentyl)carbamate compound i-71a (170 mg, 0.42 mmol) in MeOH (10 mL) at 25° C. and it was stirred at 60° C. for 3 h. The reaction mixture was concentrated, diluted with EtOAc (150 mL) and washed sequentially with sat. brine (3×50 mL). The organic layer was dried over Na₂SO₄, filtered and evaporated and the obtained residue purified by preparative TLC (MeOH:DCM=1:20) to give the title compound (140 mg, 98%) as a yellow solid. MS (ESI): m/z [M+H]⁺ 305.9.

### Intermediate 78

### Step A. i-78a

### 3-Chloro-1-(4-methoxybenzyl)pyrazin-2 (1H)-one

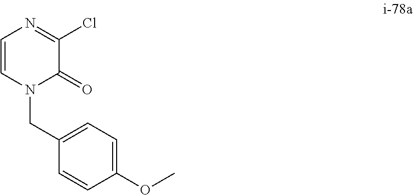 [SMILES: COc1ccc(Cn2ccnc(Cl)c2=O)cc1]

4-Methoxybenzyl chloride (720 mg, 4.60 mmol) was added to a mixture of 3-chloro-2 (1H)-pyrazinone (CAS Reg. No. 105985-17-9) (500 mg, 3.83 mmol) and TEA (1.1 mL, 7.7 mmol) in MeCN (20 mL) and the resulting mixture was stirred at 60° C. for 4 h. The reaction mixture was poured into water (50 mL) and concentrated under reduced pressure. The residue was extracted with EtOAc (3×50 mL) and the combined organic layers were dried over Na₂SO₄, filtered and evaporated. The crude material was purified by preparative TLC (EtOAc:PE=2:1) to give the title compound (200 mg, 21%) as a white solid. MS (ESI): m/z [M+H]⁺ 250.9.

### Step B. i-78b

### 6′-(((1S,3S)-3-((4-(4-Methoxybenzyl)-3-oxo-3,4-dihydropyrazin-2-yl)amino)cyclopentyl)amino)-2H-[1,3′-bipyridin]-2-one

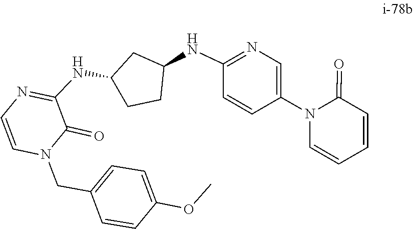 [SMILES: COc1ccc(Cn2ccnc(N[C@H]3CC[C@H](Nc4ccc(-n5ccccc5=O)cn4)C3)c2=O)cc1]

According to GM2 6′-(((1S,3S)-3-aminocyclopentyl)amino)-2H-[1,3′-bipyridin]-2-one compound i-1c (388 mg, 1.44 mmol), 3-chloro-1-(4-methoxybenzyl)pyrazin-2 (1H)-one compound i-78a (300 mg, 1.20 mmol), Pd-PEPPSI-IpentCl 2-methylpyridine (101 mg, 0.12 mmol) and Cs₂CO₃ (780 mg, 2.39 mmol) were reacted in 1,4-dioxane (15 mL) at 100° C. for 8 h to give upon Celite filtration, aqueous work-up and purification by preparative TLC (DCM:MeOH=15:1) the title compound (230 mg, 40%) as a brown solid. MS (ESI): m/z [M+H]⁺ 485.2.

### Intermediate 79

### Step A. i-79a

### 3-Chloro-1-(4-methoxybenzyl)-6-methylpyrazin-2 (1H)-one

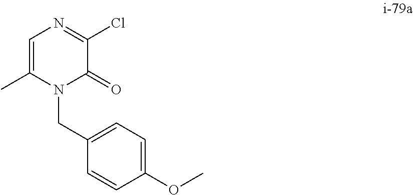

4-Methoxybenzyl chloride (650 mg, 4.15 mmol) was added to a mixture of 3-chloro-6--methylpyrazin-2(1H)-one (CAS Reg. No. 89283-34-1; F. Chillemi, G. Palamidassi, Farmaco, Edizione Scientifica 1963, 18(8), 557-65) (400 mg, 2.77 mmol) and K₃PO₄ (1.1 mL, 7.7 mmol) in DMF (15 mL) and the resulting mixture was stirred at 60° C. for 3 h. The reaction mixture was quenched with sat. brine (125 mL), extracted with EtOAc (3×100 mL) and the combined organic layers were dried over Na₂SO₄, filtered and evaporated. The crude material was purified by preparative TLC (EtOAc:PE=2:1) to give the title compound (260 mg, 36%) as a white solid. MS (ESI): m/z [M+H]⁺ 265.1/267 (Cl isotope pattern). ¹H NMR (300 MHz, DMSO-d₆) δ ppm 2.27 (3H, s), 3.73 (3H, s), 5.23 (2H, s), 6.90-6.94 (2H, m), 7.14-7.16 (2H, m), 7.18 (1H, s).

### Step B. i-79b

### 6′-(((1S,3S)-3-((4-(4-Methoxybenzyl)-5-methyl-3-oxo-3,4-dihydropyrazin-2-yl)amino)cyclopentyl)amino)-2H-[1,3′-bipyridin]-2-one

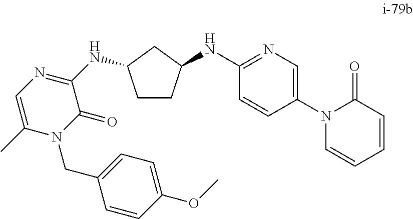 [SMILES: COc1ccc(Cn2c(C)cnc(N[C@H]3CC[C@H](Nc4ccc(-n5ccccc5=O)cn4)C3)c2=O)cc1]

According to GM2 6′-(((1S,3S)-3-aminocyclopentyl)amino)-2H-[1,3′-bipyridin]-2-one compound i-1c (102 mg, 0.38 mmol), 3-chloro-1-(4-methoxybenzyl)-6-methylpyrazin-2 (1H)-one compound i-79a (100 mg, 0.38 mmol), Pd-PEPPSI-IpentCl 2-methylpyridine (318 mg, 0.38 mmol) and K₃PO₄ (241 mg, 1.13 mmol) were reacted in 1,4-dioxane (1 mL) at 100° C. for 16 h to give upon Celite filtration, aqueous work-up and purification by preparative TLC (DCM:MeOH=10:1) the title compound (110 mg, 58%) as a white solid. ¹H NMR (300 MHz, DMSO-d₆) δ ppm 1.56 (2H, m), 1.86-2.00 (2H, m), 2.05-2.11 (2H, m), 2.14 (3H, s), 3.72 (3H, s), 4.05-4.37 (2H, m), 5.14 (2H, s), 6.27 (1H, t), 6.45 (1H, d), 6.54 (1H, d), 6.65 (1H, d), 6.81 (1H, d), 6.87-6.97 (3H, m), 7.08-7.18 (2H, m), 7.40 (1H, dd), 7.48 (1H, dt), 7.60 (1H, dd), 7.93 (1H, d).

### Intermediate 82

### Step A. i-82a

### 6-((6-Chloropyridin-3-yl)amino)-5-nitronicotinonitrile

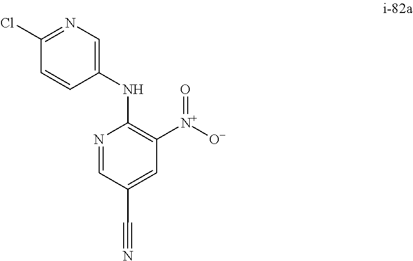 [SMILES: N#Cc1cnc(Nc2ccc(Cl)nc2)c([N+](=O)[O-])c1]

Na₂CO₃ (1.73 g, 16.3 mmol) was added to a mixture of 6-chloro-5-nitronicotinonitrile (CAS Reg. No. 160906-98-9) (1.00 g, 5.45 mmol), 6-chloropyridin-3-amine (CAS Reg. No. 5350-93-6) (1.40 g, 10.9 mmol), XantPhos (CAS Reg. No. 161265-03-8) (315 mg, 0.54 mmol) and Pd(OAc)₂ (61 mg, 0.27 mmol) in 1,4-dioxane (15 mL) at rt under nitrogen and it was stirred at 80° C. for 3 h. The reaction mixture was filtered through a filter membrane, concentrated under reduced pressure and the crude material was purified by preparative TLC (EtOAc:PE=1:1) to give the title compound (1.28 g, 85%) as a yellow solid. MS (ESI): m/z [M+H]⁺ 275.8/278 (C₁ isotope pattern).

### Step B. i-82b

### 5-Amino-6-((6-chloropyridin-3-yl)amino)nicotinonitrile

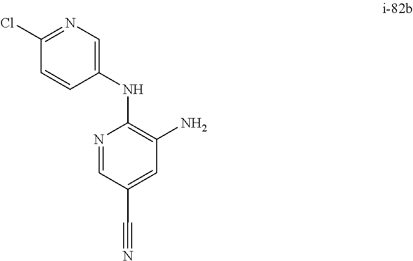 [SMILES: N#Cc1cnc(Nc2ccc(Cl)nc2)c(N)c1]

6-((6-Chloropyridin-3-yl)amino)-5-nitronicotinonitrile compound i-82a (1.28 g, 4.64 mmol) was added to a mixture of zinc (1.52 g, 23.2 mmol) and acetic acid (2.79 g, 46.4 mmol) in EtOH (15 mL) at rt and it was stirred at 60° C. for 2 h. The reaction mixture was filtered through a filter membrane, the filtrate concentrated under reduced pressure and the crude material purified by preparative TLC (EtOAc:PE=1:1) to give the title compound (1.26 g, 100%) as a yellow solid. MS (ESI): m/z [M+H]⁺ 246.0/248 (Cl isotope pattern).

### Step C. i-82c

### 3-(6-Chloropyridin-3-yl)-2-oxo-2,3-dihydro-1H-imidazo[4,5-b]pyridine-6-carbonitrile

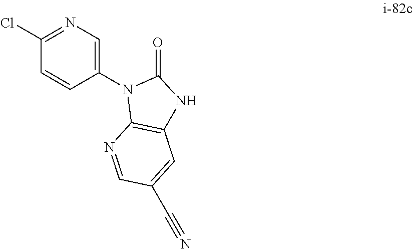 [SMILES: N#Cc1cnc2c(c1)[nH]c(=O)n2-c1ccc(Cl)nc1]

5-Amino-6-((6-chloropyridin-3-yl)amino)nicotinonitrile compound i-82b (1.26 g, 5.13 mmol) in DMF (15 mL) was treated with CDI (CAS Reg. No. 530-62-1) (4.16 g, 25.6 mmol) at rt under nitrogen and it was stirred at 80° C. for 2 h. The reaction mixture was poured into water (100 mL) and the mixture extracted with EtOAc (3×75 mL). The combined organic layers were dried over Na₂SO₄, filtered and evaporated and the crude material purified by preparative TLC (DCM:MeOH=10:1) to give the title compound (590 mg, 38%) as a yellow oil. MS (ESI): m/z [M+H]⁺ 271.9/274 (Cl isotope pattern).

### Step D. i-82d

### 3-(6-Chloropyridin-3-yl)-1-methyl-2-oxo-2,3-dihydro-1H-imidazo[4,5-b]pyridine-6-carbonitrile

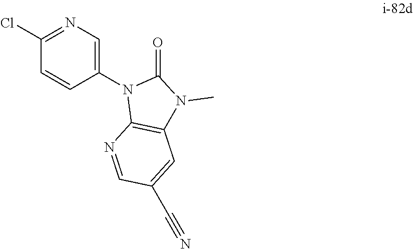 [SMILES: Cn1c(=O)n(-c2ccc(Cl)nc2)c2ncc(C#N)cc21]

In a vial a mixture of 3-(6-chloropyridin-3-yl)-2-oxo-2,3-dihydro-1H-imidazo[4,5-b]pyridine-6-carbonitrile compound i-82c (580 mg, 2.13 mmol) and K₂CO₃ (885 mg, 6.40 mmol) in DMF (2 mL) was treated with iodomethane (3.03 g, 21.4 mmol) at rt under nitrogen and it was stirred at 60° C. for 1 h. The reaction mixture was poured into water (100 mL) and the mixture extracted with EtOAc (3×75 mL). The combined organic layers were dried over Na₂SO₄, filtered and evaporated and the crude material purified by preparative TLC (DCM:PE=10:1) to give the title compound (401 mg, 66%) as a brown oil. MS (ESI): m/z [M+H]⁺ 285.9/288 (Cl isotope pattern).

### Intermediate 88

### Step A. i-88a

### Methyl 5-(((1S,3S)-3-((tert-butoxycarbonyl)amino)cyclopentyl)amino)pyrazine-2-carboxylate

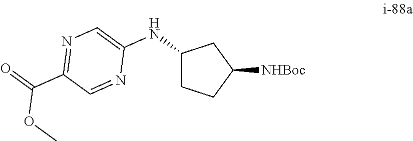 [SMILES: COC(=O)c1cnc(N[C@H]2CC[C@H](NC(=O)OC(C)(C)C)C2)cn1]

Na₂CO₃ (553 mg, 5.22 mmol) was added to tert-butyl ((1S,3S)-3-aminocyclopentyl)carbamate (CAS Reg. No. 645400-44-8) (418 mg, 2.09 mmol) and methyl 5-chloropyrazine-2-carboxylate (CAS Reg. No. 33332-25-1) (300 mg, 1.74 mmol) in DMSO (20 mL) at rt and the resulting mixture was stirred at 120° C. for 16 h. The reaction mixture was concentrated under reduced pressure, diluted with EtOAc (250 mL) and washed sequentially with brine (3×125 mL). The organic layer was dried (Na₂SO₄), filtered and evaporated and the residue purified by preparative TLC (EtOAc) to afford (533 mg, 91%) of the title compound as a pale-yellow solid. MS (ESI): m/z [M+H]⁺ 337.

### Step B. i-88b

### tert-Butyl ((1S,3S)-3-((5-(methylcarbamoyl)pyrazin-2-yl)amino)cyclopentyl)carbamate

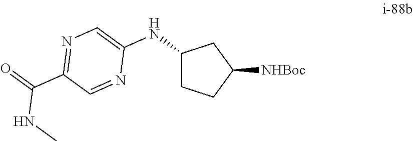 [SMILES: CNC(=O)c1cnc(N[C@H]2CC[C@H](NC(=O)OC(C)(C)C)C2)cn1]

Methyl 5-(((1S,3S)-3-((tert-butoxycarbonyl)amino)cyclopentyl)amino) pyrazine-2-carboxylate compound i-88a (533 mg, 1.58 mmol) was dissolved in methanamine (30% w/w) in EtOH (15 mL) and placed into a microwave tube. The tube was sealed and heated to 100° C. for 2 h in a microwave reactor. The solvent was removed under reduced pressure to afford (525 mg, 99%) of the crude title compound as a yellow solid which was used without further purification. MS (ESI): m/z [M+H]⁺ 335.9.

### Step C. i-88c

### 5-(((1S,3S)-3-Aminocyclopentyl)amino)-N-methylpyrazine-2-carboxamide

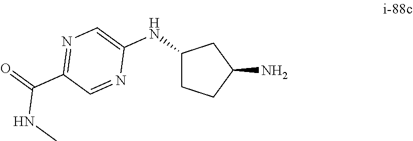 [SMILES: CNC(=O)c1cnc(N[C@H]2CC[C@H](N)C2)cn1]

HCl (4 M in dioxane, 2.5 mL, 10 mmol) was added to crude tert-butyl ((1S,3S)-3-((5-(methylcarbamoyl)pyrazin-2-yl)amino)cyclopentyl)carbamate compound i-88b (525 mg, 1.57 mmol) in MeOH (10 mL) at rt and the resulting mixture was stirred at 60° C. for 4 h. The solvent was removed under reduced pressure and the residue diluted with EtOAc (250 mL) and washed sequentially with saturated NaHCO₃ (3×100 mL). The organic layer was dried (Na₂SO₄), filtered and evaporated and the obtained material purified by preparative TLC (EtOAc:PE=1:1) to afford (425 mg, 115%) of the title compound as a pale-yellow solid. MS (ESI): m/z [M+H]⁺ 235.8.

### Intermediate 89

### i-89a

### Methyl 5-(((1S,3S)-3-((3-methoxy-2-oxo-2H-[1,3′-bipyridin]-6′-yl)amino)cyclopentyl)amino)pyrazine-2-carboxylate

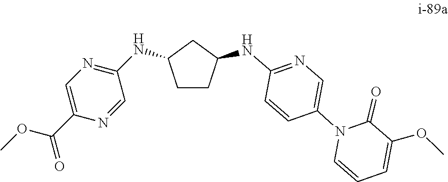

Methyl 5-chloropyrazine-2-carboxylate (CAS Reg. No. 33332-25-1) (190 mg, 1.10 mmol) was added to the 5×TFA salt of 6′-(((1S,3S)-3-aminocyclopentyl)amino)-3-methoxy-2H-[1,3′-bipyridin]-2-one compound i-3b (480 mg, 0.55 mmol), Pd-PEPPSI-IpentCl 2-methylpyridine (CAS Reg. No. 1612891-29-8) (4.6 mg, 5.5 μmol) and Cs₂CO₃ (539 mg, 1.65 mmol) in 1,4-dioxane (15 mL) at rt and the resulting mixture was stirred at 100° C. for 15 h under nitrogen. The reaction mixture was poured into water (125 mL) and extracted with EtOAc (4×100 mL). The combined organic layers were dried (Na₂SO₄), filtered and evaporated the residue was purified by preparative TLC (EtOAc) to afford (164 mg, 68%) of the title compound as a yellow solid. MS (ESI): m/z [M+H]⁺ 436.8.

### Intermediate 93

### i-93a

### 6′-(((1S,3S)-3-((5,6-Dimethylpyrazin-2-yl)amino)cyclopentyl)amino)-5-(1-(4-methoxybenzyl)-1H-tetrazol-5-yl)-2H-[1,3′-bipyridin]-2-one

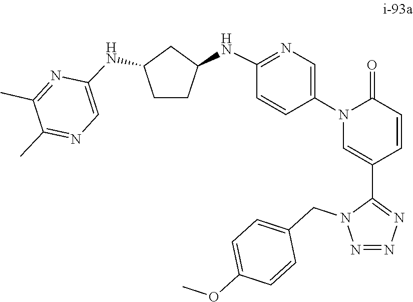 [SMILES: COc1ccc(Cn2nnnc2-c2ccc(=O)n(-c3ccc(N[C@H]4CC[C@H](Nc5cnc(C)c(C)n5)C4)nc3)c2)cc1]

Cs₂CO₃ (371 mg, 1.14 mmol) was added to 6′-chloro-5-(1-(4-methoxybenzyl)-1H-tetrazol-5-yl)-2H-[1,3′-bipyridin]-2-one compound i-65b (90 mg, 0.23 mmol), (1S,3S)—N¹-(5,6-dimethylpyrazin-2-yl)cyclopentane-1,3-diamine×3 TFA compound i-5b (187 mg, 0.34 mmol) and Pd-PEPPSI-IpentCl 2-methylpyridine (CAS:1612891-29-8) (9.6 mg, 0.01 mmol) in 1,4-dioxane (5 mL) at rt and the resulting mixture was stirred at 100° C. for 15 h under nitrogen. The reaction mixture was diluted with water (50 mL) and extracted with EtOAc (3×100 mL). The organic layers were combined, dried (Na₂SO₄), filtered and evaporated and the residue was purified by preparative TLC (MeOH:DCM=1:30) to afford (111 mg, 86%) of the title compound as a pale-yellow solid. MS (ESI): m/z [M+H]⁺ 564.9.

### Intermediate 94

### i-94a

### 5-(1-(4-Methoxybenzyl)-1H-tetrazol-5-yl)-6′-(((1S,3S)-3-((5-methylpyrazin-2-yl)amino)cyclopentyl)amino)-2H-[1,3′-bipyridin]-2-one

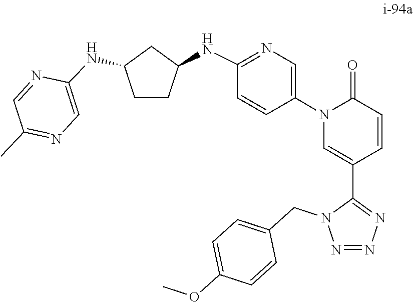 [SMILES: COc1ccc(Cn2nnnc2-c2ccc(=O)n(-c3ccc(N[C@H]4CC[C@H](Nc5cnc(C)cn5)C4)nc3)c2)cc1]

Prepared in a similar way as described for compound i-93a from (1S,3S)—N¹-(5-methylpyrazin-2-yl)cyclopentane-1,3-diamine×5 TFA compound i-31b (261 mg, 0.34 mmol), 6′-chloro-5-(1-(4-methoxybenzyl)-1H-tetrazol-5-yl)-2H-[1,3′-bipyridin]-2-one compound i-65b (90 mg, 0.23 mmol), Cs₂CO₃ (371 mg, 1.14 mmol) and Pd-PEPPSI-IpentCl 2-methylpyridine (9.6 mg, 0.01 mmol) in 1,4-dioxane (5 mL) and purified by preparative TLC (MeOH:DCM=1:30) to give the title compound (80 mg, 64%) as a beige solid. MS (ESI): m/z [M+H]⁺ 550.9.

### Intermediate 95

### i-95a

### 6′-(((1S,3S)-3-((5-(Difluoromethoxy)pyrazin-2-yl)amino)cyclopentyl)amino)-5-(1-(4-methoxybenzyl)-1H-tetrazol-5-yl)-2H-[1,3′-bipyridin]-2-one

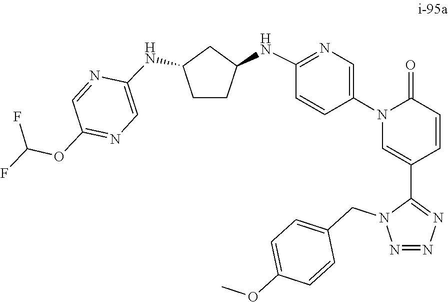 [SMILES: COc1ccc(Cn2nnnc2-c2ccc(=O)n(-c3ccc(N[C@H]4CC[C@H](Nc5cnc(OC(F)F)cn5)C4)nc3)c2)cc1]

Prepared in a similar way as described for compound i-93a from (1S,3S)—N¹-(5-(difluoromethoxy)pyrazin-2-yl)cyclopentane-1,3-diamine×3 TFA compound i-19c (134 mg, 0.23 mmol), 6′-chloro-5-(1-(4-methoxybenzyl)-1H-tetrazol-5-yl)-2H-[1,3′-bipyridin]-2-one compound i-65b (90 mg, 0.23 mmol), Cs₂CO₃ (371 mg, 1.14 mmol) and Pd-PEPPSI-IpentCl 2-methylpyridine (9.6 mg, 0.01 mmol) in 1,4-dioxane (5 mL) and purified by preparative TLC (EtOAc) to give the title compound (123 mg, 90%) as a pale-yellow solid. MS (ESI): m/z [M+H]⁺ 602.9.

### Intermediate 96

### Step A. i-96a

### tert-Butyl ((1S,3S)-3-((5-(1-(4-methoxybenzyl)-1H-tetrazol-5-yl)-2-oxo-2H-[1,3′-bipyridin]-6′-yl)amino)cyclopentyl)carbamate

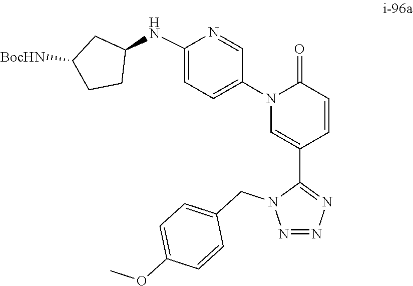 [SMILES: COc1ccc(Cn2nnnc2-c2ccc(=O)n(-c3ccc(N[C@H]4CC[C@H](NC(=O)OC(C)(C)C)C4)nc3)c2)cc1]

Pd-PEPPSI-IPentCl 2-methylpyridine (CAS Rg. No. 1612891-29-8) (0.179 g, 0.21 mmol) was added to a mixture of 6′-chloro-5-(1-(4-methoxybenzyl)-1H-tetrazol-5-yl)-2H-[1,3′-bipyridin]-2-one compound i-65b (0.84 g, 2.13 mmol), Cs₂CO₃ (1.37 g, 4.26 mmol) and tert-butyl ((1S,3S)-3-aminocyclopentyl)carbamate (CAS Reg. No 645400-44-8) (0.43 g, 2.13 mmol) in DMF (13 mL) and it was stirred under an atmosphere of nitrogen at 100° C. for 18 h. The reaction mixture was filtered through Celite and the filter cake washed with EtOAc (20 mL). The combined filtrates were concentrated under reduced pressure and the residue was purified by flash chromatography on silica (gradient: 0-10% MeOH in DCM) to afford (0.70 g, 59%) of the title compound as a yellow solid. MS (ESI): m/z [M+H]⁺ 559.3.

### Step B. i-96b

### 6′-(((1S,3S)-3-Aminocyclopentyl)amino)-5-(1-(4-methoxybenzyl)-1H-tetrazol-5-yl)-2H-[1,3′-bipyridin]-2-one

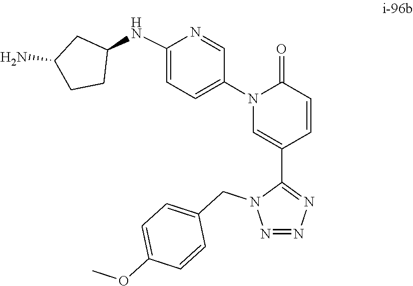 [SMILES: COc1ccc(Cn2nnnc2-c2ccc(=O)n(-c3ccc(N[C@H]4CC[C@H](N)C4)nc3)c2)cc1]

TFA (3 mL, 39 mmol) was added to tert-butyl ((1S,3S)-3-((5-(1-(4-methoxybenzyl)-1H-tetrazol-5-yl)-2-oxo-2H-[1,3′-bipyridin]-6′-yl)amino)cyclopentyl)carbamate compound i-96a (690 mg, 1.24 mmol) in DCM (10 mL) at rt and it was stirred under an atmosphere of nitrogen at rt for 2 h. The solvent was removed under reduced pressure to give an unspecified TFA salt of the title compound (600 mg, 85%) as an orange gum which was used without further purification. MS (ESI): m/z [M+H]⁺ 459.0.

### Step C. i-96c

### 6′-(((1S,3S)-3-((5-Chloropyrazin-2-yl)amino)cyclopentyl)amino)-5-(1-(4-methoxybenzyl)-1H-tetrazol-5-yl)-2H-[1,3′-bipyridin]-2-one

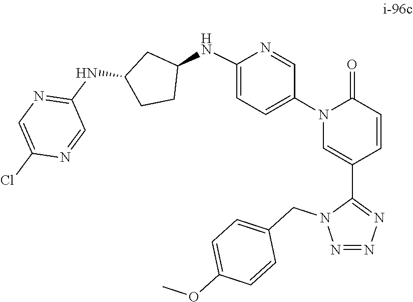 [SMILES: COc1ccc(Cn2nnnc2-c2ccc(=O)n(-c3ccc(N[C@H]4CC[C@H](Nc5cnc(Cl)cn5)C4)nc3)c2)cc1]

A mixture of 6′-(((1S,3S)-3-aminocyclopentyl)amino)-5-(1-(4-methoxybenzyl)-1H-tetrazol-5-yl)-2H-[1,3′-bipyridin]-2-one compound i-96b (150 mg, 0.33 mmol), 2,5-dichloropyrazine (CAS Reg. No. 19745-07-4) (49 mg, 0.33 mmol), Cs₂CO₃ (320 mg, 0.98 mmol) and Pd-PEPPSI-IPentCl 2-methylpyridine (CAS:1612891-29-8) (27 mg, 0.03 mmol) in 1,4-dioxane (8 mL) was stirred under an atmosphere of nitrogen at 100° C. for 18 h. The reaction mixture was quenched with water (75 mL) and extracted with EtOAc (3×50 mL). The combined organic layers were dried (Na₂SO₄), filtered and evaporated and the residue was purified by preparative TLC (EtOAc:PE=1:0) to afford (50 mg, 27%) of the title compound as a pale-yellow solid. MS (ESI): m/z [M+H]⁺ 571.3.

### Intermediate 97

### i-97a

### 6′-Fluoro-3-methoxy-2H-[1,3′-bipyridin]-2-one

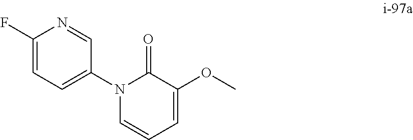 [SMILES: COc1cccn(-c2ccc(F)nc2)c1=O]

A 500 mL flask was charged with 2-fluoro-5-iodopyridine (CAS Reg. No. 171197-80-1) (11.84 g, 53.10 mmol), 3-methoxypyridin-2 (1H)-one (CAS Reg. No. 20928-63-6) (6.64 g, 53.1 mmol), Cu(I)I (2.022 g, 10.62 mmol) and K₂CO₃ (14.68 g, 106.19 mmol). rel-(1R,2R)—N¹,N²-Dimethylcyclohexane-1,2-diamine (1.68 mL, 10.6 mmol) followed by dioxane (200 mL) were added, the flask was backfilled with nitrogen (3×) and the resulting mixture stirred at 100° C. for 24 h. The mixture was cooled to rt, diluted with water (200 mL) and concentrated under reduced pressure. The residue was taken up with EtOAc (200 mL), the phases were separated and the aqueous layer was extracted with EtOAc (2×50 mL). The combined organic layers were washed with 10% citric acid (50 mL) and brine (50 mL) and concentrated under reduced pressure to give the crude title compound (5.46 g, 46%) as a beige solid (91% pure). MS (ESI): m/z [M+H]⁺ 221.2.

### Intermediate 98

### Step A. i-98a

### rel-tert-Butyl ((1R,3R)-3-((5-Methylpyrazin-2-yl)amino)cyclopentyl)carbamate

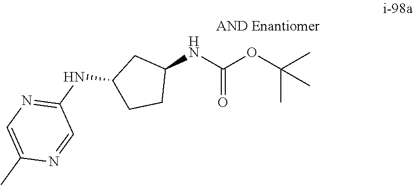 [SMILES: Cc1cnc(N[C@H]2CC[C@H](NC(=O)OC(C)(C)C)C2)cn1]

2-Chloro-5-methylpyrazine (CAS Reg. No. 59303-10-5) (0.349 g, 2.71 mmol), rel-tert-butyl ((1R,3R)-3-aminocyclopentyl)carbamate (CAS Reg. No. 947732-58-3) (0.544 g, 2.72 mmol), Pd-PEPPSI™-IPent catalyst (CAS Reg. No. 1158652-41-5) (91 mg, 0.11 mmol), Cs₂CO₃ (1.33 g, 4.08 mmol) and 1,4-dioxane (6 mL) were mixed at rt. The vial was capped, degassed and filled with nitrogen (2×) and the mixture stirred at 100° C. for 4 h under a nitrogen atmosphere. The mixture was cooled to rt and filtered through a small plug of silica. The plug was washed with EtOAc and the filtrate was evaporated. The residue was suspended in EtOAc:heptane=1:4 (10 mL) and sonicated for 5 min. The solid was filtered off, washed with EtOAc:heptane=1:4 (50 mL) followed by heptane (10 mL) and dried under vacuum overnight to give the title compound (545 mg, 81%) as a grey/white solid. MS (ESI): m/z [M+H]⁺ 293.2.

### Step B. i.98b

### rel-(1R,3R)—N¹-(5-Methylpyrazin-2-yl)cyclopentane-1,3-diamine 3TFA

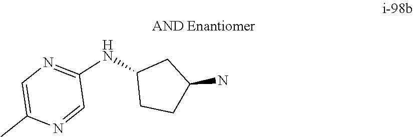

TFA (2.0 mL, 26 mmol) was added to a slurry of rel-tert-butyl ((1R,3R)-3-((5-methylpyrazin-2-yl)amino)cyclopentyl)carbamate compound i-98a (0.545 g, 1.86 mmol) in DCM (2 mL) and the resulting solution was stirred at rt for 1 h. Toluene and MeOH were added and the solvents were evaporated. The residue was co-evaporated with MeOH and toluene (combined) twice to give the crude title compound (0.878 g, 89%) as a brown oil that contained 5 mol % toluene. MS (ESI): m/z [M+H]⁺ 193.1.

### Intermediate 99

### i-99a

### Methyl 2-((5-bromopyrazin-2-yl)thio)acetate

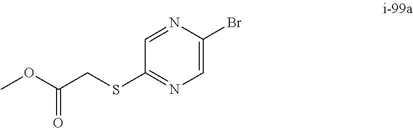

2,5-Dibromopyrazine (CAS Reg. No. 23229-26-7) (500 mg, 2.10 mmol) was added to methyl 2-mercaptoacetate (44.6 mg, 0.42 mmol) and TEA (117 μL, 0.84 mmol) in DMF (5 mL) at 20° C. over a period of 1 min under a nitrogen atmosphere and the resulting suspension was stirred at 60° C. for 15 h. The reaction mixture was concentrated under reduced pressure, the residue diluted with EtOAc (200 mL) and washed sequentially with sat. brine (3×150 mL). The organic layer was dried over Na₂SO₄, filtered and evaporated and the obtained residue purified by preparative TLC (EtOAc:PE=1:3) to give the title compound (106 mg, 96%) as a yellow solid. MS (ESI): m/z [M+H]⁺ 262.8.

### Intermediate 100

### i-100a

### 6′-Fluoro-2H-[1,3′-bipyridin]-2-one

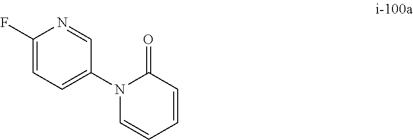 [SMILES: O=c1ccccn1-c1ccc(F)nc1]

rel-(1R,2R)—N¹,N²-Dimethylcyclohexane-1,2-diamine (1.50 g, 10.5 mmol) was added to a mixture of pyridin-2 (1H-one (CAS Reg. No. 142-08-5) (5.0 g, 52.6 mmol), 2-fluoro-5-iodopyridine (CAS Reg. No. 171197-80-1) (17.6 g, 78.9 mmol), Cu(I)I (4.01 g, 21.0 mmol) and K₂CO₃ (14.53 g, 105.2 mmol) in 1,4-dioxane (50 mL) and it was stirred at 90° C. for 18 h under a nitrogen atmosphere. The solvent was removed under reduced pressure and the residue purified by flash chromatography on silica (gradient: 0-50% EtOAc in PE) to give the title compound (11.6 g, 116%) as a brown solid. MS (ESI): m/z [M+H]⁺ 191.1. ¹H NMR (300 MHz, DMSO-d₆) δ ppm 6.36 (1H, dt), 6.52 (1H, d), 7.36 (1H, dd), 7.54 (1H, ddd), 7.73 (1H, dd), 8.14 (1H, ddd), 8.35 (1H, dd).

### Intermediate 101

### Step A. i-101a

### 1-(6-(Benzyloxy)pyridin-3-yl)-4-methylpiperazine

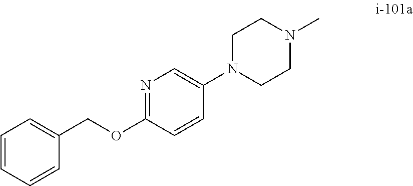 [SMILES: CN1CCN(c2ccc(OCc3ccccc3)nc2)CC1]

Sodium tert-butoxide (437 mg, 4.55 mmol) was added to a mixture of 2-(benzyloxy)-5-bromopyridine (CAS Reg. No. 83664-33-9) (400 mg, 1.51 mmol), BINAP (94 mg, 0.15 mmol), Pd₂(dba)₃ (139 mg, 0.15 mmol) and 1-methylpiperazine (455 mg, 4.54 mmol) in 1,4-dioxane (7 mL) at 25° C. The resulting solution was stirred at 120° C. for 12 min under nitrogen in a microwave reactor. The reaction mixture was filtered through Celite and the filter cake washed with DCM (2×5 mL). The combined filtrates were concentrated under reduced pressure and the obtained material was purified by C18-flash chromatography (gradient: 0-29% MeCN in water+1% TFA) to afford the title compound (303 mg, 71%) as a brown oil which solidified on standing. MS (ESI): m/z [M+H]⁺ 284. ¹H (300 MHz, DMSO-d₆) δ ppm 2.88 (5H, d), 3.23-4.00 (6H, m), 5.27 (2H, s), 6.82 (1H, d), 7.21-7.49 (6H, m), 7.84 (1H, d).

### Step B. i-101b

### 5-(4-Methylpiperazin-1-yl)pyridin-2-ol

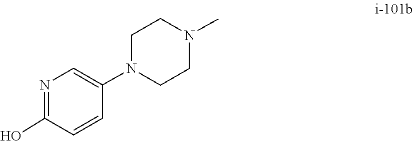

Pd—C (22.5 mg, 0.21 mmol) was added to 1-(6-(benzyloxy)pyridin-3-yl)-4-methylpiperazine compound i-101a (300 mg, 1.06 mmol) in MeOH (20 mL) at 25° C. and the resulting suspension was stirred at this temperature for 16 h under a hydrogen atmosphere. The mixture was filtered through a Celite pad and the filter cake washed with methanol (3×5 mL). The combined filtrates were concentrated under reduced pressure to afford the title compound (180 mg, 88%) as a black solid which was directly used in the next step without further purification. MS (ESI): m/z [M+H]⁺ 194.

### Intermediate 102

### i-102a

### 6′-Chloro-2′-methyl-2H-[1,3′-bipyridin]-2-one

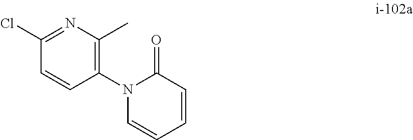

The reaction was run in 2 parallel batches (1×starting from 2 g and 1×from 1 g of boronic acid). N,N,N′,N′-Tetramethylethylenediamine (2.71 g, 23.3 mmol) was added to a mixture of pyridin-2-ol (3.33 g, 35.0 mmol), (6-chloro-2-methylpyridin-3-yl)boronic acid (2.0 g, 11.7 mmol) and copper(II) trifluoromethanesulfonate (8.44 g, 23.3 mmol) in DCM (10 mL) at 25° C. and it was stirred at 25° C. for 18 h. The two reaction mixtures were combined, evaporated to dryness, re-dissolved in EtOAc (100 mL) and washed sequentially with sat. brine (5×25 mL). The combined organic layers were dried over Na₂SO₄, filtered and evaporated and the residue was purified by preparative TLC (EtOAc:PE=1:1) to afford the title compound (36 mg, 0.9%) as a brown gum. MS (ESI): m/z [M+H]⁺ 220.90. ¹H NMR (300 MHz, DMSO-d₆) δ ppm 2.20 (3H, s), 6.37 (1H, dt), 6.52 (1H, dt), 7.48-7.57 (2H, m), 7.60 (1H, ddd), 7.82 (1H, d).

### Intermediate 103

### Step A. i-103a

### tert-Butyl ((1S,3S)-3-((5-(hydroxymethyl)-2-oxo-2H-[1,3′-bipyridin]-6′-yl)amino)cyclopentyl)carbamate

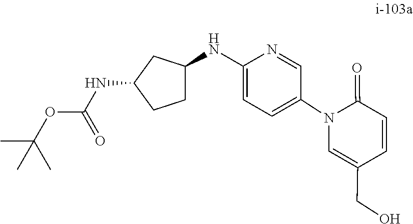 [SMILES: CC(C)(C)OC(=O)N[C@H]1CC[C@H](Nc2ccc(-n3cc(CO)ccc3=O)cn2)C1]

K₃PO₄ (395 mg, 1.86 mmol) was added to a mixture of tert-butyl ((1S,3S)-3-((5-iodopyridin-2-yl)amino)cyclopentyl)carbamate compound i-1a (250 mg, 0.62 mmol), 5-(hydroxymethyl)pyri-din-2 (1H-one (CAS Reg. No. 109205-68-1) (155 mg, 1.24 mmol), Cu(I)I (59 mg, 0.31 mmol) and N,N-dimethylethane-1,2-diamine (27 mg, 0.31 mmol) in DMF (5 mL) at 20° C. under a nitrogen atmosphere and it was stirred at 100° C. for 16 h. The reaction mixture was poured into water (100 mL) and the aqueous layer was extracted with EtOAc (2×100 mL). The combined organic layers were washed with sat. brine (3×50 mL), dried over Na₂SO₄, filtered and evaporated and the residue was purified by preparative TLC (7 M NH₃ in MeOH:DCM=1:20) to give the title compound (155 mg, 62%) as a purple solid. MS (ESI): m/z [M+H]⁺ 401.1.

### Step B. i-103b

### tert-Butyl ((1S,3S)-3-((5-(chloromethyl)-2-oxo-2H-[1,3′-bipyridin]-6′-yl)amino)cyclopentyl)carbamate

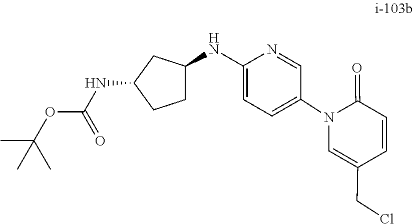 [SMILES: CC(C)(C)OC(=O)N[C@H]1CC[C@H](Nc2ccc(-n3cc(CCl)ccc3=O)cn2)C1]

Thionyl chloride (430 mg, 3.6 mmol) was added to a mixture of tert-butyl ((1S,3S)-3-((5-(hydroxymethyl)-2-oxo-2H-[1,3′-bipyridin]-6′-yl)amino)cyclopentyl)carbamate compound i-103a (144 mg, 0.36 mmol) in DCM (10 mL) at 20° C. and it was stirred at this temperature for 16 h. The reaction mixture was concentrated under reduced pressure and the obtained crude title compound (355 mg) was directly used in the next step without further purification. MS (ESI): m/z [M+H]⁺ 419/421 (Cl isotope pattern).

### Step C. i-103c

### 6′-(((1S,3S)-3-Aminocyclopentyl)amino)-5-((methylthio)methyl)-2H-[1,3′-bipyridin]-2-one

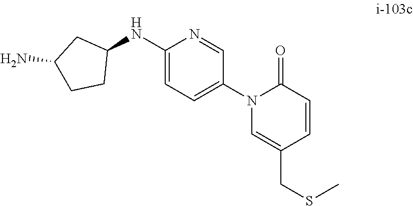 [SMILES: CSCc1ccc(=O)n(-c2ccc(N[C@H]3CC[C@H](N)C3)nc2)c1]

Sodium thiomethoxide (173 mg, 2.47 mmol) was added to a mixture of crude tert-butyl ((1S,3S)-3-((5-(chloromethyl)-2-oxo-2H-[1,3′-bipyridin]-6′-yl)amino)cyclopentyl)carbamate compound i-103b (345 mg) in EtOH (10 mL) at 20° C. and it was stirred at 60° C. for 16 h. The reaction mixture was concentrated under reduced pressure and the obtained material was purified by preparative TLC (7 M NH₃ in MeOH:DCM=1:20) to give the title compound (88 mg, 32%) as a brown solid (along with 20 mg of the Boc-protected title compound). MS (ESI): m/z [M+H]⁺ 331.1.

### Step D. i-103d

### 6′-(((1S,3S)-3-((5-Methylpyrazin-2-yl)amino)cyclopentyl)amino)-5-((methylthio)methyl)-2H-[1,3′-bipyridin]-2-one

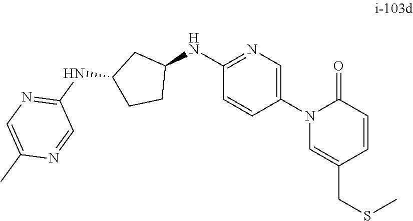

In a slight variation of GM2 Cs₂CO₃ (231 mg, 0.71 mmol) was added to a mixture of 6′-(((1S,3S)-3-aminocyclopentyl)amino)-5-((methylthio)methyl)-2H-[1,3′-bipyridin]-2-one compound i-103c (78 mg, 0.24 mmol), 2-bromo-5-methylpyrazine (CAS Reg. No. 98006-90-7) (82 mg, 0.47 mmol) and Pd-PEPPSI-IpentCl 2-methylpyridine (99 mg, 0.12 mmol) in DMF (3 mL) at 20° C. and the resulting mixture stirred at 100° C. for 16 h under nitrogen. The reaction mixture was poured into sat. brine (100 mL) and the aqueous layer was extracted with EtOAc (3×100 mL). The combined organic layers were washed with sat. brine (3×50 mL), dried over Na₂SO₄, filtered and evaporated and the residue was purified by preparative TLC (7 M NH₃ in MeOH:DCM=1:20) to give the title compound (76 mg, 76%) as a brown solid. MS (ESI): m/z [M+H]⁺ 423.1.

### Intermediate 106

### Step A. i-106a

### tert-Butyl ((1S,3S)-3-((5-(5-cyano-6-oxopyridazin-1 (6H)-yl)pyridin-2-yl)amino)cyclo-pentyl)carbamate

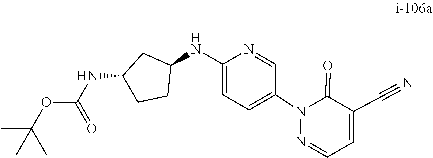 [SMILES: CC(C)(C)OC(=O)N[C@H]1CC[C@H](Nc2ccc(-n3nccc(C#N)c3=O)cn2)C1]

tert-Butyl ((1S,3S)-3-((5-(5-chloro-6-oxopyridazin-1 (6H)-yl)pyridin-2-yl)amino)cyclopentyl)carbamate compound i-70a (420 mg, 1.03 mmol) was added to a mixture of Pd₂(dba)₃ (95 mg, 0.10 mmol), dppf (574 mg, 1.03 mmol), zinc powder (135 mg, 2.07 mmol) and zinc cyanide (243 mg, 2.07 mmol) in DMF (15 mL) at 20° C. under a nitrogen atmosphere and the mixture was stirred at 100° C. for 15 h. The reaction mixture was quenched with sat. brine (150 mL) and extracted with EtOAc (3×100 mL). The combined organic layers were dried over Na₂SO₄, filtered and evaporated and the residue was purified by preparative TLC (MeOH:DCM=1:10) to give the title compound (232 mg, 57%) as an orange solid. MS (ESI): m/z [M+H]⁺ 397.0.

### Step B. i-106b

### 2-(6-(((1S,3S)-3-Aminocyclopentyl)amino)pyridin-3-yl)-3-oxo-2,3-dihydropyridazine-4-carbonitrile

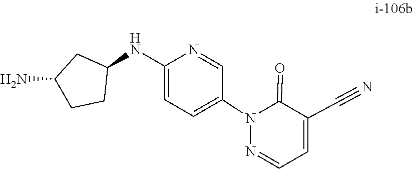 [SMILES: N#Cc1ccnn(-c2ccc(N[C@H]3CC[C@H](N)C3)nc2)c1=O]

A mixture of tert-butyl ((1S,3S)-3-((5-(5-cyano-6-oxopyridazin-1 (6H)-yl)pyridin-2-yl)amino)cyclo-pentyl)carbamate compound i-106a (200 mg, 0.50 mmol) in DCM (3 mL) was treated with TFA (1 mL) at 20° C. and it was stirred at 20° C. for 3 h. The reaction mixture was concentrated under reduced pressure to give an unspecified TFA salt of the title compound (370 mg, 97%) as a brown oil which was used without further purification. ¹H NMR (400 MHz, DMSO-d₆) δ ppm 1.53-1.64 (2H, m), 1.87-1.95 (1H, m), 1.98-2.04 (1H, m), 2.11-2.18 (2H, m), 3.63-3.72 (1H, m), 4.32-4.39 (1H, m), 6.64 (1H, d), 7.56 (1H, brs), 7.65 (1H, dd), 7.91 (2H, brs), 8.19 (1H, d), 8.22-8.24 (2H, m).

### Intermediate 107

### Step A. i-107a

### 4-Bromo-6-chloro-2-(4-methoxybenzyl)pyridazin-3 (2H)-one

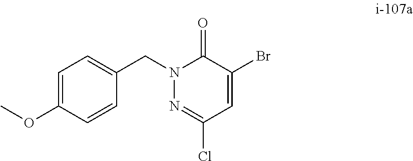 [SMILES: COc1ccc(Cn2nc(Cl)cc(Br)c2=O)cc1]

1-(Chloromethyl)-4-methoxybenzene (7.48 g, 47.8 mmol) was added to a mixture of 4-bromo-6-chloro-3 (2H-pyridazinone (CAS Reg. No. 933041-13-5) (5.0 g, 24 mmol) and Cs₂CO₃ (15.6 g, 47.8 mmol) in MeCN (80 mL) at 15° C. and it was stirred at 60° C. for 3 h. The mixture was filtered through a pad of Celite, the filter cake washed with DCM (3×20 mL) and the combined filtrates were concentrated under reduced pressure. The obtained material was triturated with EtOAc:PE=1:5 (25 mL), the formed solid collected by filtration and dried under vacuum to give the title compound (7.8 g, 98%) as a yellow solid. MS (ESI): m/z [M+H]⁺ 329/331 (Br/Cl isotope pattern).

### Step B. i-107b

### 6-Chloro-4-hydroxy-2-(4-methoxybenzyl)pyridazin-3 (2H)-one

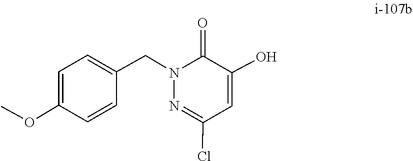 [SMILES: COc1ccc(Cn2nc(Cl)cc(O)c2=O)cc1]

RockPhos Pd G3 (CAS Reg. No. 2009020-38-4) (1.97 g, 2.35 mmol) was added to a mixture of 4-bromo-6-chloro-2-(4-methoxybenzyl)pyridazin-3 (2H)-one compound i-107a (7.75 g, 23.5 mmol), (E)-benzaldehyde oxime (3.70 g, 30.6 mmol) and Cs₂CO₃ (15.6 g, 47.8 mmol) in DMF (40 mL) and water (10 mL) at 15° C. and it was stirred at 80° C. for 15 h. The reaction mixture was poured into sat. brine (100 mL) and the aqueous layer was extracted with EtOAc (3×100 mL). The combined organic layers were washed with sat. brine (3×100 mL), dried over Na₂SO₄, filtered and evaporated and the residue was purified by flash chromatography on silica (gradient: 0-100% EtOAc in PE) to give the title compound (4.0 g, 64%) as a tan solid. MS (ESI): m/z [M+H]⁺ 267/269 (Cl isotope pattern).

### Step C. i-107c

### 6-Chloro-4-(difluoromethoxy)-2-(4-methoxybenzyl)pyridazin-3 (2H)-one

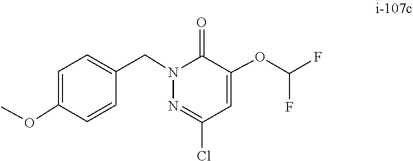 [SMILES: COc1ccc(Cn2nc(Cl)cc(OC(F)F)c2=O)cc1]

Cs₂CO₃ (293 mg, 0.90 mmol) was added to a mixture of 6-chloro-4-hydroxy-2-(4-methoxybenzyl)pyridazin-3 (2H)-one compound i-107b (200 mg, 0.75 mmol) and it was stirred at 20° C. for 1.5 h. Sodium chlorodifluoroacetate (CAS Reg. No. 1895-39-2) (343 mg, 2.25 mmol) was added and the resulting mixture was stirred at 100° C. for 3.5 h. The reaction mixture was poured into sat. brine (50 mL) and the aqueous layer was extracted with EtOAc (3×50 mL). The combined organic layers were washed with sat. brine (3×50 mL), dried over Na₂SO₄, filtered and evaporated and the residue was purified by reparative TLC (EtOAc:PE=1:3) to give the title compound (175 mg, 74%) as a yellow gum. MS (ESI): m/z [M+H]⁺ 317/319 (Cl isotope pattern).

### Step D. i-107d

### 4-(Difluoromethoxy)-2-(4-methoxybenzyl)pyridazin-3 (2H)-one

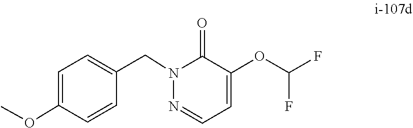 [SMILES: COc1ccc(Cn2nccc(OC(F)F)c2=O)cc1]

Pd—C (11 mg, 0.11 mmol) was added to a mixture of 6-chloro-4-(difluoromethoxy)-2-(4-methoxybenzyl)pyridazin-3 (2H)-one compound i-107c (680 mg, 2.15 mmol) in MeOH (30 mL) at 20° C. and the resulting suspension was stirred at this temperature for 2 h under a hydrogen atmosphere. The reaction mixture was concentrated under reduced pressure to afford the title compound (576 mg, 95%) as a white solid which was directly used in the next step without further purification. MS (ESI): m/z [M+H]⁺ 283.0.

### Step E. i-107e

### 4-(Difluoromethoxy)pyridazin-3 (2H)-one

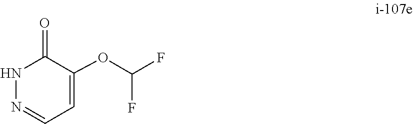 [SMILES: O=c1[nH]nccc1OC(F)F]

TFA (20 mL) was added to 4-(difluoromethoxy)-2-(4-methoxybenzyl)pyridazin-3 (2H)-one compound i-107d (546 mg, 1.93 mmol) at 20° C. and the mixture was stirred at 80° C. for 15 h. The reaction mixture was concentrated under reduced pressure to give an unspecified TFA salt of the title compound (483 mg, 90%) as a black gum which was used without further purification. MS (ESI): m/z [M+H]⁺ 162.9.

### EXAMPLES

### Example 1

### 3-Methyl-5-(((1S,3S)-3-((2-oxo-2H-[1,3′-bipyridin]-6′-yl)amino)cyclopentyl)amino)pyrazine-2-carbonitrile—compound 1

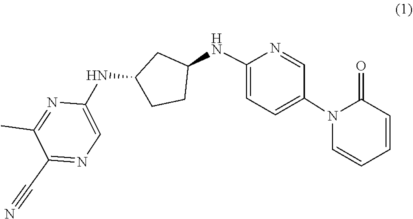 [SMILES: Cc1nc(N[C@H]2CC[C@H](Nc3ccc(-n4ccccc4=O)cn3)C2)cnc1C#N]

According to GM1A 5-chloro-3-methylpyrazine-2-carbonitrile (CAS Reg. No. 1260665-75-5) (85 mg, 0.55 mmol), 6′-(((1S,3S)-3-aminocyclopentyl)amino)-2H-[1,3′-bipyridin]-2-one compound i-1c (150 mg, 0.55 mmol) and TEA (232 μL, 1.66 mmol) were reacted in DMSO (5 mL) at 110° C. for 18 h. The mixture was evaporated and the crude product was purified by preparative HPLC (PrepMethod B, gradient: 23-53%) to give the title compound (70 mg, 32%) as a yellow solid. HRMS (ESI) m/z [M+H]⁺ calcd for C₂₁H₂₂N₇O: 388.1880, found: 388.1878. ¹H NMR (400 MHz, DMSO-d₆) δ ppm 1.48-1.59 (2H, m) 1.87-1.99 (2H, m) 2.11-2.24 (2H, m) 2.43 (3H, s) 4.31-4.41 (1H, m) 6.28 (1H, td) 6.45 (1H, d) 6.53 (1H, d) 6.98 (1H, d) 7.41 (1H, dd) 7.48 (1H, ddd) 7.61 (1H, dd) 7.81 (1H, br s) 7.93 (1H, d) 8.25 (1H, br d).

### Example 2

### 6′-(((1S,3S)-3-((5,6-Dimethylpyrazin-2-yl)amino)cyclopentyl)amino)-2H-[1,3′-bipyridin]-2-one—compound 2

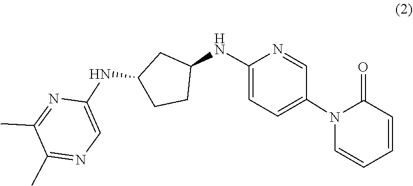 [SMILES: Cc1ncc(N[C@H]2CC[C@H](Nc3ccc(-n4ccccc4=O)cn3)C2)nc1C]

In a variation of GM2 5-chloro-2,3-dimethylpyrazine (CAS Reg. No. 59489-32-6) (132 mg, 0.92 mmol) was added to 6′-(((1S,3S)-3-aminocyclopentyl)amino)-2H-[1,3′-bipyridin]-2-one compound i-1c (250 mg, 0.92 mmol), Cs₂CO₃ (904 mg, 2.77 mmol) and Pd₂(dba)₃-CHCl₃ adduct (CAS Reg. No. 52522-40-4) (191 mg, 0.18 mmol) in toluene (5 mL) and the reaction mixture stirred at 90° C. for 18 h under a nitrogen atmosphere. The reaction mixture was diluted with EtOAc (20 mL) and washed with water (3×50 mL). The organic layer was dried over Na₂SO₄, filtered and evaporated. The residue was purified by preparative TLC (MeOH:DCM, 1:10) followed by preparative HPLC (PrepMethod B, gradient: 15-42%) to give the title compound (40 mg, 11%) as a white solid. HRMS (ESI) m/z [M+H]⁺ calcd for C₂₁H₂₅N₆O: 377.2084, found: 377.2078. ¹H NMR (300 MHz, DMSO-d₆) δ ppm 1.40-1.56 (2H, m) 1.88 (2H, t) 2.07-2.22 (2H, m) 2.25 (6H, d) 4.15-4.42 (2H, m) 6.26 (1H, td) 6.44 (1H, d) 6.52 (1H, d) 6.64 (1H, d) 6.91 (1H, d) 7.39 (1H, dd) 7.47 (1H, ddd) 7.56-7.64 (2H, m) 7.92 (1H, d).

### Example 3

### 6′-(((1S,3S)-3-((5-Chloropyrazin-2-yl)amino)cyclopentyl)amino)-2H-[1,3′-bipyridin]-2-one—compound 3

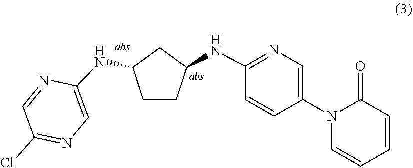 [SMILES: O=c1ccccn1-c1ccc(N[C@H]2CC[C@H](Nc3cnc(Cl)cn3)C2)nc1]

According to GM2 2,5-dichloropyrazine (CAS Reg. No. 19745-07-4) (100 mg, 0.67 mmol), 6′-(((1S,3S)-3-aminocyclopentyl)amino)-2H-[1,3′-bipyridin]-2-one compound i-1c (363 mg, 1.34 mmol), Pd-PEPPSI-IpentCl 2-methylpyridine (452 mg, 0.54 mmol) and Cs₂CO₃ (656 mg, 2.01 mmol) were reacted in 1,4-dioxane (5 mL) at 100° C. for 18 h to give upon aqueous work-up and purification by preparative HPLC (PrepMethod B, gradient 25-55%) the title compound (20 mg, 8%) as a white solid. HRMS (ESI) m/z [M+H]⁺ calcd for C₁₉H₂₀ClN₆O: 383.1382, found: 383.1396. ¹H NMR (300 MHz, DMSO-d₆) δ ppm 1.51 (1H, br d), 1.89 (2H, t), 2.08-2.20 (2H, m), 4.07-4.51 (2H, m), 6.28 (1H, t), 6.46 (1H, s), 6.53 (1H, d), 6.95 (1H, d), 7.38-7.51 (3H, m), 7.61 (1H, dd), 7.73 (1H, d), 7.93 (1H, d), 8.04 (1H, d).

### Example 4

### 6′-(((1S,3S)-3-((5-Methylpyrazin-2-yl)amino)cyclopentyl)amino)-2H-[1,3′-bipyridin]-2-one—compound 4

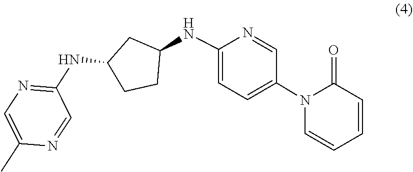 [SMILES: Cc1cnc(N[C@H]2CC[C@H](Nc3ccc(-n4ccccc4=O)cn3)C2)cn1]

In a variation of GM2 2-bromo-5-methylpyrazine (CAS Reg. No. 98006-90-7) (0.026 g, 0.15 mmol) was transferred into a vial and put into a glovebox. Sodium tert-butoxide (0.072 g, 0.75 mmol) followed by 6′-(((1S,3S)-3-aminocyclopentyl)amino)-2H-[1,3′-bipyridin]-2-one compound i-1c (0.057 g, 0.15 mmol), tBuXPhos Pd G3 (CAS Reg. No. 1447963-75-8) (0.012 g, 0.02 mmol) and tBuXPhos (CAS Reg. No. 564483-19-8) (3.2 mg, 7.5 μmol) as a solution in DMA (1 mL) was added inside the glovebox and the mixture was stirred at 65° C. for 24 h. After cooling to rt the reaction mixture was concentrated, re-dissolved in DMSO and purified by prep. HPLC to give the title compound (6.6 mg, 12%). HRMS (ESI) m/z [M+H]⁺ calcd for C₂₀H₂₂N₆O: 363.1933, found: 363.1935. ¹H NMR (600 MHz, DMSO-d₆) δ ppm 1.43-1.55 (2H, m), 1.88 (2H, t), 2.11-2.19 (2H, m), 2.24 (3H), 4.26 (1H, q), 4.33 (1H, p), 6.27 (1H, td), 6.45 (1H, dt), 6.53 (1H, d), 6.87 (1H, d), 6.94 (1H, d), 7.40 (1H, dd), 7.48 (1H, ddd), 7.61 (1H, dd), 7.82 (2H, dd), 7.93 (1H, d).

### Example 5

### 5-(((1S,3S)-3-((2-Oxo-2H-[1,3′-bipyridin]-6′-yl)amino)cyclopentyl)amino)pyrazine-2-carbonitrile—compound 5

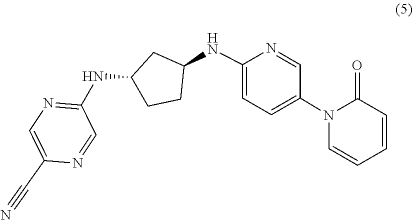 [SMILES: N#Cc1cnc(N[C@H]2CC[C@H](Nc3ccc(-n4ccccc4=O)cn3)C2)cn1]

According to GM1A 5-chloropyrazine-2-carbonitrile (CAS Reg. No. 36070-75-4) (62 mg, 0.44 mmol), 6′-(((1S,3S)-3-aminocyclopentyl)amino)-2H-[1,3′-bipyridin]-2-one compound i-1c (100 mg, 0.37 mmol) and TEA (155 μL, 1.11 mmol) were reacted in DMSO (5 mL) at 110° C. for 18 h to give upon non-aqueous work-up and purification by prep. HPLC (PrepMethod A, gradient: 25-50%) the title compound (47 mg, 34%) as a yellow solid. HRMS (ESI) m/z [M+H]⁺ calcd for C₂₀H₁₉N₇O: 374.1724, found: 374.1726. ¹H NMR (400 MHz, DMSO-d₆) δ ppm 1.59-1.49 (2H, m), 1.93 (2H, t), 2.23-2.13 (2H, m), 4.36 (2H, q), 6.28 (1H, td), 6.45 (1H, dt), 6.53 (1H, d), 6.98 (1H, d), 7.41 (1H, dd), 7.48 (1H, ddd), 7.63-7.58 (1H, m), 7.98-7.92 (2H, m), 8.36 (1H, d), 8.48 (1H, d).

### Example 6

### 6′-(((1S,3S)-3-((5-Acetylpyrazin-2-yl)amino)cyclopentyl)amino)-2H-[1,3′-bipyridin]-2-one—compound 6

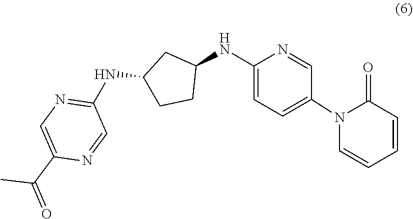 [SMILES: CC(=O)c1cnc(N[C@H]2CC[C@H](Nc3ccc(-n4ccccc4=O)cn3)C2)cn1]

According to GM1A 1-(5-chloropyrazin-2-yl)ethan-1-one (CAS Reg. No. 160252-31-3) (70 mg, 0.44 mmol), 6′-(((1S,3S)-3-aminocyclopentyl)amino)-2H-[1,3′-bipyridin]-2-one compound i-1c (100 mg, 0.37 mmol) and Na₂CO₃ (118 mg, 1.11 mmol) were reacted in DMSO (5 mL) at 100° C. for 18 h to give upon aqueous work-up and purification by prep. HPLC (PrepMethod C, gradient: 1-39%) the title compound (38 mg, 26%) as a white solid. HRMS (ESI) m/z [M+H]⁺ calcd for C₂₁H₂₃N₆O₂: 391.1876, found: 391.1868. ¹H NMR (300 MHz, DMSO-d₆) δ ppm 1.56 (2H, d), 1.94 (2H, t), 2.19 (2H, dd), 2.46 (3H, s), 4.49-4.33 (2H, m), 6.49 (2H, dd), 6.96 (1H, d), 7.52-7.37 (2H, m), 7.60 (1H, dd), 7.94 (2H, d), 8.14 (1H, d), 8.55 (1H, d).

### Example 7

### 6′-(((1S,3S)-3-((5-Methyl-5H-pyrrolo[2,3-b]pyrazin-2-yl)amino)cyclopentyl)amino)-2H-[1,3′-bipyridin]-2-one—compound 7

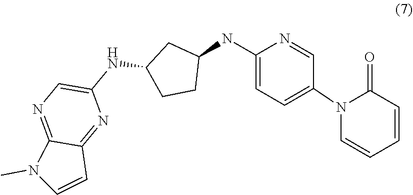

According to GM2 6′-(((1S,3S)-3-aminocyclopentyl)amino)-2H-[1,3′-bipyridin]-2-one compound i-1c (130 mg, 0.48 mmol), 2-bromo-5-methyl-5H-pyrrolo[2,3-b]pyrazine (CAS Reg. No. 1217090-32-8) (153 mg, 0.72 mmol), Pd-PEPPSI-IpentCl 2-methylpyridine (20 mg, 0.02 mmol) and Cs₂CO₃ (783 mg, 2.40 mmol) were reacted in 1,4-dioxane (10 mL) at 100° C. for 15 h to give upon non-aqueous work-up and purification by preparative TLC (MeOH:DCM=1:10) followed by preparative HPLC (PrepMethod E, gradient: 2-19%) the title compound (40 mg, 21%) as a white solid. HRMS (ESI) m/z [M+H]⁺ calcd for C₂₂H₂₄N₇O: 402.2036, found: 402.2022. ¹H NMR (300 MHz, DMSO-d₆) δ ppm 1.51 (2H, td), 1.91 (2H, t), 2.18 (2H, td), 3.73 (3H, s), 4.34 (2H, d), 6.18-6.33 (2H, m), 6.45 (1H, d), 6.54 (1H, d), 6.62 (1H, d), 6.97 (1H, d), 7.36-7.55 (3H, m), 7.55-7.72 (2H, m), 7.92 (1H, d).

### Example 8

### 6′-(((1S,3S)-3-((5-Methyl-5H-pyrrolo[2,3-b]pyrazin-3-yl)amino)cyclopentyl)amino)-2H-[1,3′-bipyridin]-2-one—compound 8

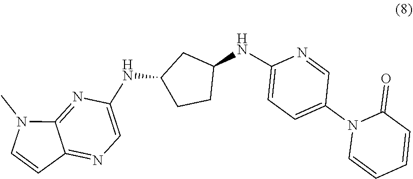 [SMILES: Cn1ccc2ncc(N[C@H]3CC[C@H](Nc4ccc(-n5ccccc5=O)cn4)C3)nc21]

Prepared in analogy to Example 7 from 6′-(((1S,3S)-3-aminocyclopentyl)amino)-2H-[1,3′-bipyridin]-2-one compound i-1c (130 mg, 0.48 mmol) and 3-chloro-5-methyl-5H-pyrrolo[2,3-b]pyrazine (CAS Reg. No. 1111638-11-9) (121 mg, 0.72 mmol) and purified by preparative TLC (MeOH:DCM=1:10) followed by preparative HPLC (PrepMethod E, gradient: 2-19%) to give a pale yellow solid (24 mg, 12%). HRMS (ESI) m/z [M+H]⁺ calcd for C₂₂H₂₄N₇O: 402.2036, found: 402.2014. ¹H NMR (300 MHz, DMSO-d₆) δ ppm 1.55 (2H, dd), 1.97 (2H, dq), 2.08-2.32 (2H, m), 3.66 (3H, s), 4.37 (2H, q), 6.27 (1H, td), 6.34 (1H, d), 6.46 (1H, t), 6.54 (1H, d), 6.96 (2H, t), 7.19 (1H), 7.41 (1H, dd), 7.48 (1H, ddd), 7.61 (1H, dd), 7.77 (1H, s), 7.93 (1H, d).

### Example 9

### 6′-(((1S,3S)-3-((1-Methyl-1H-pyrazolo[3,4-b]pyrazin-6-yl)amino)cyclopentyl)amino)-2H-[1,3′-bipyridin]-2-one—compound 9

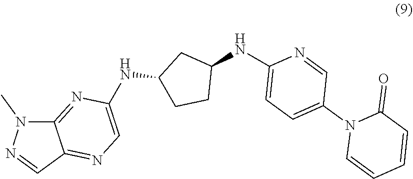 [SMILES: Cn1ncc2ncc(N[C@H]3CC[C@H](Nc4ccc(-n5ccccc5=O)cn4)C3)nc21]

Prepared in analogy to Example 7 from 6′-(((1S,3S)-3-aminocyclopentyl)amino)-2H-[1,3′-bipyridin]-2-one compound i-1c (100 mg, 0.37 mmol) and 6-chloro-1-methyl-1H-pyrazolo[3,4-b]pyrazine compound i-34a (118 mg, 0.55 mmol) and purified by preparative TLC (MeOH:DCM=1:10) followed by preparative HPLC (PrepMethod E, gradient: 3-25%) to give a white solid (11 mg, 7%). HRMS (ESI) m/z [M+H]⁺ calcd for C₂₁H₂₃N₈O: 403.1990, found: 403.1960. ¹H NMR (300 MHz, DMSO-d₆) δ ppm 1.42-1.74 (2H, m), 1.99 (2H, q), 2.23 (2H, dd), 3.84 (3H, s), 4.40 (2H, dt), 6.28 (1H, td), 6.40-6.51 (1H, m), 6.55 (1H, d), 7.00 (1H, d), 7.29-7.53 (2H, m), 7.61 (1H, dd), 7.84 (1H, d), 7.89-8.00 (3H, m).

### Example 11

### 1-(6-(((1S,3S)-3-((5,6-Dimethylpyrazin-2-yl)amino)cyclopentyl)amino)pyridin-3-yl)-3-methyl-1,3-dihydro-2H-benzo[d]imidazol-2-one—compound 11

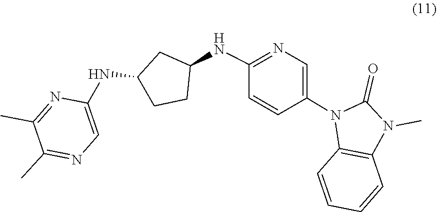

According to GM2 5-chloro-2,3-dimethylpyrazine (77 mg, 0.54 mmol), 1-(6-(((1S,3S)-3-aminocyclopentyl)amino)pyridin-3-yl)-3-methyl-1,3-dihydro-2H-benzo[d]imidazol-2-one compound i-2b (100 mg, 0.18 mmol), Pd-PEPPSI-IpentCl 2-methylpyridine (12 mg, 0.01 mmol) and Cs₂CO₃ (292 mg, 0.90 mmol) were reacted in 1,4-dioxane (3 mL) at 100° C. for 18 h to give upon non-aqueous work-up (filter cake washed with DCM) and purification by preparative TLC (7 M in NH₃ in MeOH:DCM=1:20) followed by preparative HPLC (PrepMethod C, gradient: 27-57%) the title compound (25 mg, 32%) as a white solid. HRMS (ESI) m/z [M+H]⁺ calcd for C₂₄H₂₈N₇O: 430.2350, found: 430.2356. ¹H NMR (300 MHz, DMSO-d₆) δ ppm 1.41-1.60 (2H, m) 1.90 (2H, t) 2.10-2.23 (2H, m) 2.27 (6H, d) 3.39 (3H, s) 4.20-4.44 (2H, m) 6.61 (1H, d) 6.67 (1H, d) 6.85-6.91 (1H, m) 6.97 (1H, d) 7.04 (1H, td) 7.12 (1H, td) 7.20-7.25 (1H, m) 7.46 (1H, dd) 7.63 (1H, s) 8.05 (1H, d).

### Example 12

### 6′-(((1S,3S)-3-((5,6-Dimethylpyrazin-2-yl)amino)cyclopentyl)amino)-3-methoxy-2H-[1,3′-bipyridin]-2-one—compound 12

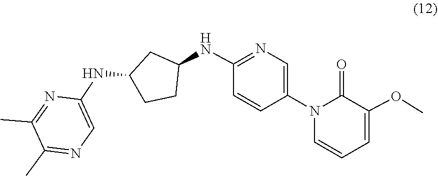

Prepared in analogy to Example 11 from 6′-(((1S,3S)-3-aminocyclopentyl)amino)-3-methoxy-2H-[1,3′-bipyridin]-2-one 2HCl compound i-3b (150 mg, 0.40 mmol) and 5-chloro-2,3-dimethylpyrazine (CAS Reg. No. 59489-32-6) (115 mg, 0.80 mmol) and applying preparative HPLC (PrepMethod C, gradient: 14-44%) to give a white solid (69 mg, 42%). HRMS (ESI) m/z [M+H]⁺ calcd for C₂₂H₂₇N₆O₂: 407.2190, found: 407.2186. ¹H NMR (300 MHz, DMSO-d₆) δ ppm 1.48 (2H, m) 1.88 (2H, t) 2.09-2.21 (2H, m) 2.26 (6H, d) 3.73 (3H, s) 4.29 (2H, m) 6.20 (1H, t) 6.53 (1H, br d) 6.67 (1H, br d) 6.86 (1H, br d) 6.93 (1H, br d) 7.16 (1H, br d) 7.38 (1H, dd) 7.62 (1H, s) 7.91 (1H, d).

### Example 13

### 6′-(((1S,3S)-3-((5-Chloropyrazin-2-yl)amino)cyclopentyl)amino)-3-methoxy-2H-[1,3′-bipyridin]-2-one—compound 13

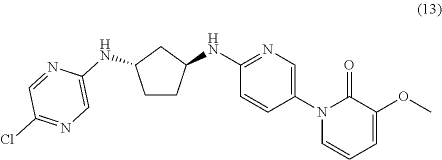 [SMILES: COc1cccn(-c2ccc(N[C@H]3CC[C@H](Nc4cnc(Cl)cn4)C3)nc2)c1=O]

Prepared in analogy to Example 11 from 6′-(((1S,3S)-3-aminocyclopentyl)amino)-3-methoxy-2H-[1,3′-bipyridin]-2-one 2HCl compound i-3b (150 mg, 0.40 mmol) and 2,5-dichloropyrazine (239 mg, 1.61 mmol) and applying preparative HPLC (PrepMethod A, gradient 40-50%) to give a white solid (33 mg, 20%). HRMS (ESI) m/z [M+H]⁺ calcd for C₂₀H₂₂ClN₆O₂: 413.1488, found: 413.1482. ¹H NMR (300 MHz, DMSO-d₆) δ ppm 1.24 (2H, br s), 1.89 (2H, br t), 2.16 (2H, br s), 3.73 (3H, s), 4.13-4.48 (2H, m), 6.20 (1H, s), 6.53 (1H, d), 6.87-6.89 (1H, m), 6.93-6.95 (1H, m), 7.16-7.19 (1H, m), 7.36-7.44 (2H, m), 7.73 (1H, s), 7.92 (1H, s), 8.04 (1H, s).

### Example 14

### 6′-(((1S,3S)-3-((5,6-Dimethylpyrazin-2-yl)amino)cyclopentyl)amino)-2-oxo-2H-[1,3′-bipyridine]-5-carboxylic acid—compound 14

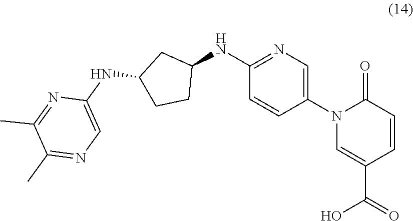 [SMILES: Cc1ncc(N[C@H]2CC[C@H](Nc3ccc(-n4cc(C(=O)O)ccc4=O)cn3)C2)nc1C]

NaOH (30 mg, 0.75 mmol) was added to methyl 6′-(((1S,3S)-3-((5,6-dimethylpyrazin-2-yl)amino)cyclopentyl)amino)-2-oxo-2H-[1,3′-bipyridine]-5-carboxylate compound i-4e (65 mg, 0.15 mmol) in a mixture of MeOH (6 mL) and water (2 mL) at 25° C. and the resulting solution stirred at this temperature for 1 h. The solvent was removed under reduced pressure and the crude product purified by preparative HPLC (PrepMethod E, gradient: 8-17%) to give the title compound (14 mg, 19%) as a white solid. HRMS (ESI) m/z [M+H]⁺ calcd for C₂₂H₂₅N₆O₃: 421.1982, found: 421.1996. ¹H NMR (400 MHz, DMSO-d₆) δ ppm 1.41-1.57 (2H, m), 1.88 (2H, t), 2.1-2.22 (2H, m), 2.26 (6H, d), 4.15-4.42 (2H, m), 6.52 (2H, dd), 6.68 (1H, d), 7.01 (1H, d), 7.44 (1H, dd), 7.62 (1H, s), 7.85 (1H, dd), 7.99 (1H, d), 8.16 (1H, d).

### Example 15

### 6′-(((1S,3S)-3-((5,6-Dimethylpyrazin-2-yl)amino)cyclopentyl)amino)-5-methoxy-2H-[1,3′-bipyridin]-2-one—compound 15

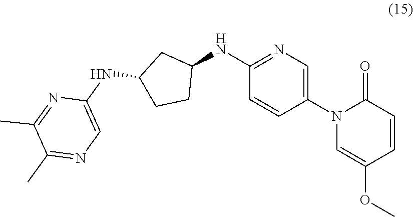

According to GM3 (1S,3S)—N-(5,6-dimethylpyrazin-2-yl)-N3-(5-iodopyridin-2-yl)cyclopentane-1,3-diamine compound i-5c (100 mg, 0.24 mmol), 5-methoxypyridin-2 (1H-one (CAS Reg. No. 61941-79-5) (153 mg, 1.22 mmol), Cu(I)I (47 mg, 0.24 mmol), Cs₂CO₃ (239 mg, 0.73 mmol) and rel-(1R,2R)—N¹,N²-dimethylcyclohexane-1,2-diamine (35 mg, 0.24 mmol) were reacted in 1,4-dioxane (5 mL) at 100° C. for 18 h to give upon aqueous work-up and purification by preparative TLC (DCM: 7 M NH₃ in MeOH=20:1) followed by preparative HPLC (PrepMethod B, gradient: 21-51%) the title compound (45 mg, 45%) as a white solid. HRMS (ESI) m/z [M+H]⁺ calcd for C₂₂H₂₇N₆O₂: 407.2190, found: 407.2166. ¹H NMR (300 MHz, DMSO-d₆) δ ppm 1.39-1.58 (2H, m), 1.81-1.97 (2H, m), 2.08-2.21 (2H, m), 2.26 (6H, d), 3.64 (3H, s), 4.16-4.40 (2H, m), 6.43 (1H, d), 6.53 (1H, d), 6.68 (1H, d), 6.92 (1H, d), 7.19 (1H, d), 7.32-7.45 (2H, m), 7.62 (1H, s), 7.97 (1H, d).

TABLE 3
The following Examples (16-19) where prepared in analogy to Example 15 starting
from compound i-5c and the given nucleophiles, applying Ullmann coupling (GM3).

| | | | Purification | Nucleophile |
|---|---|---|---|---|
| | | | Method/ | (CAS Reg. |
| Ex | Structure/Name | Analytical Data | Yield | No.) |
| 16 | 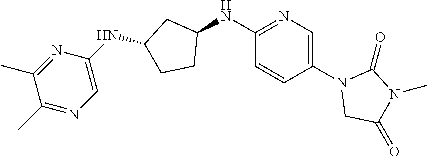 1-(6-(((1S,3S)-3-((5,6-Di- methylpyrazin-2- yl)amino)cyclopentyl)amino)pyri- din-3-yl)-3- methylimidazolidine-2,4-dione | HRMS (ESI) m/z [M + H]⁺ calcd for C₂₀H₂₆N₇O₂: 396.2142, found: 396.2124. ¹H NMR (400 MHz, DMSO-d₆) δ ppm 1.38- 1.52 (2H, m), 1.68-1.91 (2H, m), 2.07-2.22 (2H, m), 2.25 (6H, d), 2.91 (3H, s), 4.18-4.30 (2H, m), 4.38 (2H, s), 6.49 (1H, d), 6.58-6.70 (2H, m), 7.59-7.65 (2H, m), 8.09 (1H, d). | Prep. TLC (MeOH (7-M NH₃):DCM = 1:20) and prep. HPLC applying PrepMethod B (gradient: 22-42%); (96 mg, 66%) | 6843-45-4 |
| 17 | 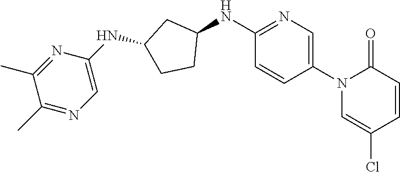 [SMILES: Cc1ncc(N[C@H]2CC[C@H](Nc3ccc(-n4cc(Cl)ccc4=O)cn3)C2)nc1C] 5-Chloro-6′-(((1S,3S)-3-((5,6- dimethylpyrazin-2- yl)amino)cyclopentyl)amino)- 2H-[1,3′-bipyridin]-2-one | HRM (ESI) m/z [M + H]⁺ calcd for C₂₁H₂₄ClN₆O: 411.1694, found: 411.1694. ¹H NMR (400 MHz, DMSO-d₆) δ ppm 1.42- 1.54 (2H, m), 1.82-1.93 (2H, m), 2.08-2.21 (2H, m), 2.26 (6H, d), 4.20- 4.37 (2H, m), 6.51 (2H, t), 6.68 (1H, d), 6.98 (1H, d), 7.41 (1H, dd), 7.55 (1H, dd), 7.62 (1H, s), 7.91 (1H, d), 7.95 (1H, d). | Prep. TLC (MeOH (7 M NH₃):DCM = 1:20) and prep. HPLC applying PrepMethod E (gradient: 9-27%); (41 mg, 40%) | 4214-79-3 |
| 18 | 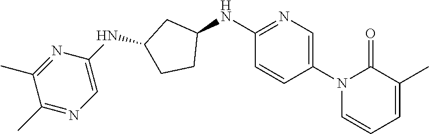 6′-(((1S,3S)-3-((5,6-Dimethyl- pyrazin-2-yl)amino)cyclo- pentyl)amino)-3-methyl-2H- [1,3′-bipyridin]-2-one | HRMS (ESI) m/z [M + H]⁺ calcd for C₂₂H₂₇N₆O: 391.2240, found: 391.2244. ¹H NMR (300 MHz, DMSO-d₆) δ ppm 1.40- 1.57 (2H, m), 1.81-1.95 (2H, m), 2.02 (3H, s), 2.05-2.22 (2H, m), 2.26 (6H, d), 4.20-4.38 (2H, m), 6.19 (1H, t), 6.52 (1H, d), 6.68 (1H, d), 6.93 (1H, d), 7.34-7.47 3H, m), 7.62 (1H, s), | Prep. TLC (MeOH:DCM = 1:20) and prep. HPLC applying PrepMethod B (gradient: 25-45%); (58 mg, 39%) | 1003-56-1 |
| | | 7.91 (1H, d). | | |
| 19 | 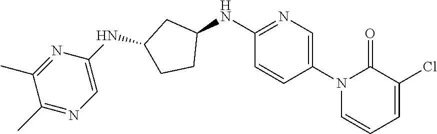 [SMILES: Cc1ncc(N[C@H]2CC[C@H](Nc3ccc(-n4cccc(Cl)c4=O)cn3)C2)nc1C] 3-Chloro-6′-(((1S,3S)-3-((5,6- dimethylpyrazin-2- yl)amino)cyclopentyl)amino)- 2H-[1,3′-bipyridin]-2-one | HRMS (ESI) m/z [M + H]⁺ calcd for C₂₁H₂₄ClN₆O: 411.1694, found: 411.1662. ¹H NMR (300 MHz, DMSO-d₆) δ ppm 1.42- 1.56 (2H, m), 1.88 (2H, t), 2.08-2.21 (2H, m), 2.26 (6H, d), 4.18-4.38 (2H, m), 6.31 (1H, t), 6.54 (1H, d), 6.69 (1H, d), 7.02 (1H, d), 7.44 (1H, dd), 7.62 (1H, s), | Prep. TLC (MeOH:DCM = 1:20) and prep. HPLC applying PrepMethod E (gradient: 10-27%); (59 mg, 28%) | 13466-35-8 |
| | | 7.67 (1H, dd), 7.82 (1H, | | |
| | | dd), 7.96 (1H, d). | | |

### Example 20

### 1-(6-(((1S,3S)-3-((5,6-Dimethylpyrazin-2-yl)amino)cyclopentyl)amino)pyridin-3-yl)-1,8-naphthyridin-2 (1H)-one—compound 20

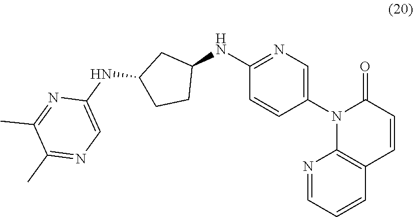 [SMILES: Cc1ncc(N[C@H]2CC[C@H](Nc3ccc(-n4c(=O)ccc5cccnc54)cn3)C2)nc1C]

According to GM2 1-(6-chloropyridin-3-yl)-1,8-naphthyridin-2 (1H)-one compound i-6a (100 mg, 0.39 mmol), (1S,3S)—N¹-(5,6-dimethylpyrazin-2-yl)cyclopentane-1,3-diamine 5TFA compound i-5b (452 mg, 0.58 mmol), Cs₂CO₃ (632 mg, 1.94 mmol) and Pd-PEPPSI-IpentCl 2-methylpyridine (16 mg, 0.02 mmol) were reacted in 1,4-dioxane (10 mL) at 100° C. for 15 h to give upon aqueous work-up and purification by preparative TLC (7 M NH₃ in MeOH:DCM=1:40) followed by preparative HPLC (PrepMethod B, gradient: 24-42%) the title compound (100 mg, 60%) as a white solid. HRMS (ESI) m/z [M+H]⁺ calcd for C₂₄H₂₆N₇O: 428.2194, found: 428.2178. ¹H NMR (300 MHz, DMSO-d₆) δ ppm 1.39-1.63 (2H, in), 1.91 (2H, t), 2.11-2.22 (2H, m), 2.26 (6H, d), 4.19-4.45 (2H, m), 6.58 (1H, d), 6.69 (1H, d), 6.76 (1H, d), 6.85 (1H, d), 7.15-7.39 (2H, m), 7.63 (1H, s), 7.79 (1H, d), 8.04 (1H, d), 8.21 (1H, dd), 8.44 (1H, dd).

### Example 21

### 3-(6-(((1S,3S)-3-((5,6-Dimethylpyrazin-2-yl)amino)cyclopentyl)amino)pyridin-3-yl)-1-methyl-1,3-dihydro-2H-imidazo[4,5-b]pyridin-2-one—compound 21

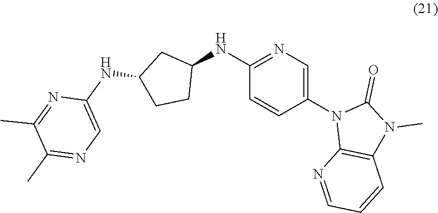

Prepared in analogy to Example 20 from 3-(6-chloropyridin-3-yl)-1-methyl-1,3-dihydro-2H-imidazo[4,5-b]pyridin-2-one compound i-24a (90 mg, 0.35 mmol) and (1S,3S)—N¹-(5,6-dimethylpyrazin-2-yl)cyclopentane-1,3-diamine 5TFA compound i-5b (291 mg, 0.38 mmol) and applying preparative TLC (7 M NH₃ in MeOH:DCM=1:25) followed by preparative HPLC (PrepMethod C, gradient: 24-42%) to give a white solid (65 mg, 43%). HRMS (ESI) m/z [M+H]⁺ calcd for C₂₃H₂₇N₈O: 431.2302, found: 431.2272. ¹H NMR (300 MHz, DMSO-d₆) δ ppm 1.40-1.61 (2H, m), 1.87-1.93 (2H, m), 2.11-2.21 (2H, m), 2.26 (6H, d), 3.41 (3H, s), 4.20-4.43 (2H, m), 6.59 (1H, d), 6.72 (1H, d), 6.93 (1H, d), 7.11-7.18 (1H, m), 7.50 (1H, dd), 7.58 (1H, dd), 7.63 (1H, s), 7.94 (1H, dd), 8.09 (1H, d).

### Example 22

### 3-(6-(((1S,3S)-3-((5,6-Dimethylpyrazin-2-yl)amino)cyclopentyl)amino)pyridin-3-yl)-1-methylimidazolidine-2,4-dione—compound 22

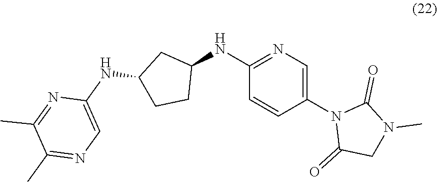

Prepared in analogy to Example 20 from 3-(6-chloropyridin-3-yl)-1-methylimidazolidine-2,4-dione compound i-7b (100 mg, 0.44 mmol) and (1S,3S)—N¹-(5,6-dimethylpyrazin-2-yl)cyclopentane-1,3-diamine 5TFA compound i-5b (438 mg, 0.58 mmol) and applying preparative TLC (7 M NH₃ in MeOH:DCM=1:20) followed by preparative HPLC (PrepMethod C, gradient: 17-35%) to give a white solid (15 mg, 8%). HRMS (ESI) m/z [M+H]⁺ calcd for C₂₀H₂₆N₇O₂: 396.2142, found: 396.2134. ¹H NMR (300 MHz, DMSO-d₆) δ ppm 1.41-1.54 (2H, m), 1.87 (2H, t), 2.10-2.20 (2H, m), 2.26 (6H, d), 2.91 (3H, s), 4.08 (2H, s), 4.20-4.36 (2H, m), 6.52 (1H, d), 6.67 (1H, d), 6.84-7.04 (1H, m), 7.28 (1H, dd), 7.62 (1H, s), 7.85 (1H, d).

### Example 23

### 6′-(((1S,3S)-3-((5,6-Dimethylpyrazin-2-yl)amino)cyclopentyl)amino)-2-oxo-2H-[1,3′-bipyridine]-3-carbonitrile—compound 23

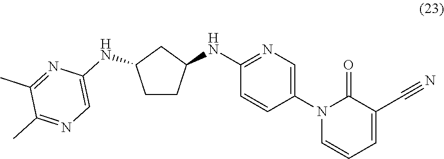 [SMILES: Cc1ncc(N[C@H]2CC[C@H](Nc3ccc(-n4cccc(C#N)c4=O)cn3)C2)nc1C]

Prepared in analogy to Example 20 from 6′-chloro-2-oxo-2H-[1,3′-bipyridine]-3-carbonitrile compound i-10a (120 mg, 0.52 mmol) and (1S,3S)—N-(5,6-dimethylpyrazin-2-yl)cyclopentane-1,3-diamine 5TFA compound i-5b (804 mg, 1.04 mmol) in DMF (10 mL) and applying preparative TLC (7 M NH₃ in MeOH:DCM=1:22) followed by a second preparative TLC (EtOAc) and further purified using preparative HPLC (PrepMethod C, gradient: 20-40%) to give the title compound (5 mg, 2%) as a yellow solid. HRMS (ESI) m/z [M+H]⁺ calcd for C₂₂H₂₄N₇O: 402.2036, found: 402.2054. ¹H NMR (300 MHz, DMSO-d₆) δ ppm 1.41-1.58 (2H, m), 1.88 (2H, m), 2.07-2.22 (2H, m), 2.26 (6H, s), 4.30 (2H, m), 6.43-6.58 (2H, m), 6.68 (1H, d), 7.06 (1H, d), 7.46 (1H, m), 7.62 (1H, s), 7.99 (1H, d), 8.06 (1H, m), 8.23 (1H, m).

### Example 24

### 6′-(((1S,3S)-3-((5-Chloropyrazin-2-yl)amino)cyclopentyl)amino)-3-methyl-2H-[1,3′-bipyridin]-2-one—compound 24

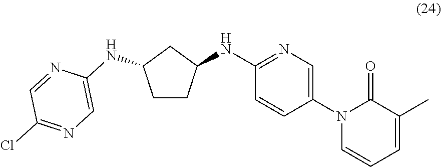 [SMILES: Cc1cccn(-c2ccc(N[C@H]3CC[C@H](Nc4cnc(Cl)cn4)C3)nc2)c1=O]

According to GM1A 6′-(((1S,3S)-3-aminocyclopentyl)amino)-3-methyl-2H-[1,3′-bipyridin]-2-one 3HCl compound i-15b (300 mg, 0.76 mmol), 2,5-dichloropyrazine (227 mg, 1.52 mmol) and Na₂CO₃ (242 mg, 2.29 mmol) were reacted in DMSO (3 mL) at 120° C. for 6 h to give upon aqueous work-up and purification by preparative TLC (MeOH:DCM=1:20) followed by preparative HPLC (PrepMethod B, gradient: 31-61%) the title compound (75 mg, 25%) as a white solid. HRMS (ESI) m/z [M+H]⁺ calcd for C₂₀H₂₂ClN₆O: 397.1538, found: 397.1510. ¹H NMR (400 MHz, DMSO-d₆) δ ppm 1.51 (2H, br s), 1.89 (2H, br t), 2.02 (3H, s), 2.11-2.22 (2H, m), 4.25 (1H, br d), 4.34 (1H, br d), 6.20 (1H, t), 6.52 (1H, d), 6.93 (1H, br d), 7.35-7.47 (4H, m), 7.73 (1H, s), 7.90-7.94 (1H, m), 8.04 (1H, s).

### Example 25

### 2-(6-(((1S,3S)-3-((5-Chloropyrazin-2-yl)amino)cyclopentyl)amino)pyridin-3-yl)pyridazin-3 (2H)-one—compound 25

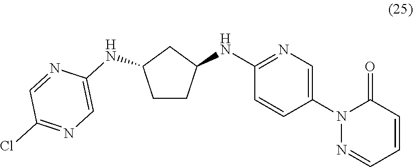 [SMILES: O=c1cccnn1-c1ccc(N[C@H]2CC[C@H](Nc3cnc(Cl)cn3)C2)nc1]

Prepared in analogy to Example 24 from 2-(6-(((1S,3S)-3-aminocyclopentyl)amino)pyridin-3-yl)pyridazin-3 (2H)-one compound i-16b (141 mg, 0.52 mmol) and 2,5-dichloropyrazine (155 mg, 1.04 mmol) at 120° C. for 16 h. The crude product was purified by preparative TLC (NH₃ in MeOH (7 M):DCM=1:20) followed by preparative HPLC (PrepMethod B, gradient: 25-55%) to give the title compound (34 mg, 17%) as a yellow solid. HRMS (ESI) m/z [M+H]⁺ calcd for C₁₈H₁₉ClN₇O: 384.1334, found: 384.1300. ¹H NMR (300 MHz, DMSO-d₆) δ ppm 1.39-1.58 (m, 2H), 1.88 (t, 2H), 2.08-2.24 (m, 2H), 4.28 (dq, 2H), 6.43-6.58 (m, 1H), 6.88-7.07 (m, 2H), 7.32-7.56 (m, 3H), 7.71 (d, 1H), 7.93-8.04 (m, 2H), 8.10 (d, 1H).

### Example 26

### 1-(6-(((1S,3S)-3-((5-Chloropyrazin-2-yl)amino)cyclopentyl)amino)pyridin-3-yl)-3-methylimidazolidine-2,4-dione—compound 26

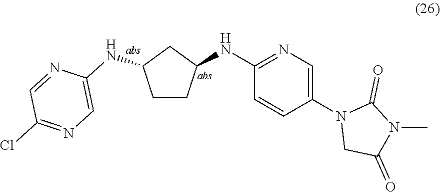 [SMILES: CN1C(=O)CN(c2ccc(N[C@H]3CC[C@H](Nc4cnc(Cl)cn4)C3)nc2)C1=O]

According to GM3 (1S,3S)—N¹-(5-chloropyrazin-2-yl)-N3-(5-iodopyridin-2-yl)cyclopentane-1,3-diamine compound i-17b (100 mg, 0.24 mmol), 3-methylimidazolidine-2,4-dione (CAS Reg. No. 6843-45-4) (137 mg, 1.20 mmol), Cu(I)I (46 mg, 0.24 mmol), Cs₂CO₃ (235 mg, 0.72 mmol) and rel-(1R,2R)—N¹,N²-dimethylcyclohexane-1,2-diamine (34 mg, 0.24 mmol) were reacted in 1,4-dioxane (3 mL) at 100° C. for 15 h to give upon non-aqueous work-up and purification by preparative TLC (7 M NH₃ in MeOH:DCM=1:20, 161 mg material isolated) followed by preparative HPLC (PrepMethod B, gradient: 27-57%) the title compound (50 mg, 52%) as a white solid. HRMS (ESI) m/z [M+H]⁺ calcd for C₁₈H₂₁ClN₇O₂: 402.1440, found: 402.1410.

¹H NMR (300 MHz, DMSO-d₆) δ ppm 1.49 (2H, br s), 1.87 (2H, br t), 2.07-2.18 (2H, 4), 2.92 (3H, s), 4.25 (2H, br d), 4.38 (2H, s), 6.50 (1H, br d), 6.62 (1H, br d), 7.39 (1H, br d), 7.64 (1H, br d), 7.73 (1H, s), 8.04 (1H, s), 8.10 (1H, br s).

TABLE 4
The following Examples (27-32) where prepared in analogy to Example 26 starting from compound i-17b and the given nucleophiles,
applying Ullmann coupling (GM3).

| | | | Purification | Nucleophile |
|---|---|---|---|---|
| | | | Method/ | (CAS |
| Ex | Structure/Name | Analytical Data | Yield | Reg. No.) |
| 27 | 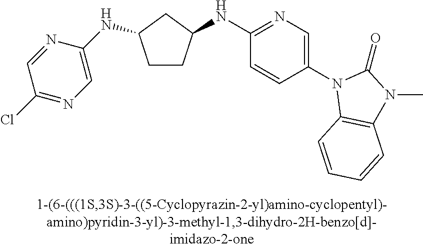 [SMILES: Cn1c(=O)n(-c2ccc(N[C@H]3CC[C@H](Nc4cnc(Cl)cn4)C3)nc2)c2ccccc21] | HRMS (ESI) m/z [M + H]⁺ calcd for C₂₂H₂₃ClN₇O: 436.1648, found: 436.1642. ¹H NMR (300 MHz, DMSO-d₆) δ ppm 1.46- 1.60 (2H, m), 1.92 (2H, t), 2.11-2.25 (2H, m), 3.36- 3.42 (3H, m), 4.22-4.42 (2H, m), 6.61 (1H, d), 6.88 (1H, d), 6.96-7.16 (3H, m), 7.20-7.25 (1H, m), 7.39- 7.49 (2H, m), 7.74 (1H, d), 8.03-8.07 (2H, m). | Prep. TLC (MeOH (7 M NH₃): DCM = 1:20) and prep. HPLC applying PrepMethod C (gradient: 32-62%); (40 mg, 38%) | 1849-01-0 |
| 28 | 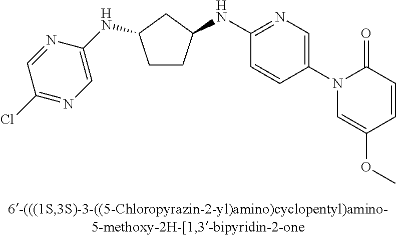 [SMILES: COc1ccc(=O)n(-c2ccc(N[C@H]3CC[C@H](Nc4cnc(Cl)cn4)C3)nc2)c1] | HRMS (ESI) m/z [M + H]⁺ calcd for C₂₀H₂₂ClN₆O₂: 413.1488, found: 413.1464. ¹H NMR (300 MHz, DMSO-d₆) δ ppm 1.45-1.59 (2H, m), 1.90 (2H, t), 2.12- 2.22 (2H, m), 3.65 (3H, s), 4.20-4.40 (2H, m), 6.43 (1H, d), 6.53 (1H, d), 6.92 (1H, d), 7.19 (1H, d), 7.33- 7.46 (3H, m), 7.73 (1H, d), 7.98 (1H, d), 8.04 (1H, d). | Prep. TLC (MeOH (7 M NH₃): DCM = 1:20) and prep. HPLC applying PrepMethod C (gradient: 20-50%); (41 mg, 41%) | 61941-79-5 |
| 29 | 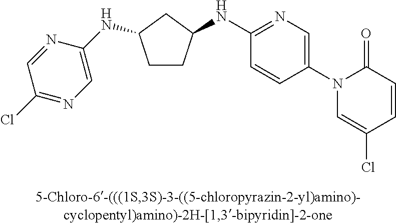 [SMILES: O=c1ccc(Cl)cn1-c1ccc(N[C@H]2CC[C@H](Nc3cnc(Cl)cn3)C2)nc1] | HRMS (ESI) m/z [M + H]⁺ calcd for C₁₉H₁₉Cl₂N₆O: 417.0992, found: 417.0968. ¹H NMR (300 MHz, DMSO-d₆) δ ppm 1.44- 1.58 (2H, m), 1.90 (2H, t), 2.11-2.22 (2H, m), 4.20- 4.38 (2H, m), 6.51 (2H, t), 6.99 (1H, d), 7.38-7.45 (2H, m), 7.55 (1H, dd), 7.73 (1H, d), 7.90 (1H, d), 7.96 (1H, d), 8.02-8.07 (1H, m). | Prep. TLC (MeOH (7 M NH₃): DCM = 1:20) and prep. HPLC applying PrepMethod B (gradient: 34-64%); (34 mg, 34%) | 4214-79-3 |
| 30 | 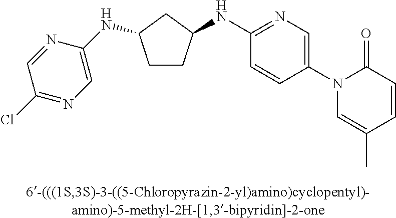 [SMILES: Cc1ccc(=O)n(-c2ccc(N[C@H]3CC[C@H](Nc4cnc(Cl)cn4)C3)nc2)c1] | HRMS (ESI) m/z [M + H]⁺ calcd for C₂₀H₂₂ClN₆O: 397.1538, found: 397.1524. ¹H NMR (300 MHz, DMSO-d₆) δ ppm 1.51 (2H, br s), 1.86-1.92 (2H, m), 2.01-2.09 (3H, m), 2.16 (2H, br s), 4.14-4.46 (2H, m), 6.39 (1H, d), 6.52 (1H, d), 6.83-7.02 (1H, m), 7.39 (4H, br d), 7.73 (1H, s), 7.92 (1H, d), 8.04 (1H, s). | Prep. TLC (MeOH (7 M NH₃): DCM = 1:20) and prep. HPLC applying PrepMethod B (gradient: 33-53%); (45 mg, 47%) | 1192-99-0 |
| 31 | 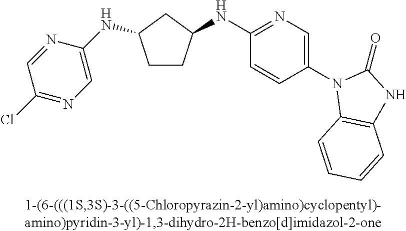 [SMILES: O=c1[nH]c2ccccc2n1-c1ccc(N[C@H]2CC[C@H](Nc3cnc(Cl)cn3)C2)nc1] | HRMS (ESI) m/z [M +H ]⁺ calcd for C₂₁H₂₁ClN₇O: 422.1490, found: 422.1462. ¹H NMR (300 MHz, DMSO-d₆) δ ppm 1.46- 1.60 (2H, m), 1.92 (2H, t), 2.11-2.24 (2H, m), 4.22- 4.30 (1H, m), 4.36 (1H, m), 6.62 (1H, d), 6.83 (1H, d), 6.95-7.07 (4H, m), 7.40- 7.50 (2H, m), 7.74 (1H, d), 8.05 (2H, d), 11.05 (1H, s). | Prep. TLC (MeOH (7 M NH₃): DCM = 1:25) and prep. HPLC applying PrepMethod C (gradient: 34-66%); (23 mg, 23%) | 615-16-7 |
| 32 | 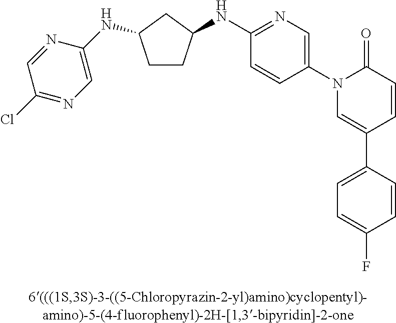 [SMILES: O=c1ccc(-c2ccc(F)cc2)cn1-c1ccc(N[C@H]2CC[C@H](Nc3cnc(Cl)cn3)C2)nc1] | HRMS (ESI) m/z [M + H]⁺ calcd for C₂₅H₂₃ClFN₆O: 477.1600, found: 477.1584. ¹H NMR (300 MHz, DMSO-d₆) δ ppm 1.52 (2H, br s), 1.91 (2H, br s), 2.17 (2H, br s), 4.27 (1H, br s), 4.36 (1H, br s), 6.57 (2H, br d), 6.99 (1H, br d), 7.24 (2H, br t), 7.37-7.55 (2H, m), 7.67 (2H, br s), 7.74 (1H, br s), 7.83 - 8.00 (2H, m), 8.04 (2H, br s). ¹⁹F NMR (282 MHz, DMSO- d₆) δ ppm-115.92 (1F, s). | Prep. TLC (MeOH (7 M NH₃): DCM = 1:20) and prep. HPLC applying PrepMethod D (gradient: 33-63%); (42 mg, 24%) | 163563- 23-3 |

### Example 33

### 6′-(((1S,3S)-3-((5-Chloropyrazin-2-yl)amino)cyclopentyl)amino)-2-oxo-2H-[1,3′-bipyridine]-5-carboxylic acid—compound 33

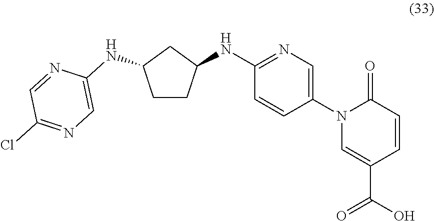 [SMILES: O=C(O)c1ccc(=O)n(-c2ccc(N[C@H]3CC[C@H](Nc4cnc(Cl)cn4)C3)nc2)c1]

NaOH (41.2 mg, 1.03 mmol) was added to methyl 6′-(((1S,3S)-3-((5-chloropyrazin-2-yl)amino)cyclopentyl)amino)-2-oxo-2H-[1,3′-bipyridine]-5-carboxylate compound i-18a (91 mg, 0.21 mmol) in a mixture of MeOH (6 mL) and water (2 mL) at 20° C. and the resulting solution stirred at this temperature for 1 h. The reaction mixture was concentrated under reduced pressure and the obtained residue diluted with water (10 mL). The solvent was adjusted to pH=3 with 1 M HCl (aq), filtered and the filtrate evaporated to afford crude product which was purified by preparative HPLC (PrepMethod C, gradient: 4-24%) to give the title compound (35 mg, 40%) as a white solid. HRMS (ESI) m/z [M+H]⁺ calcd for C₂₀H₂₀ClN₆O₃: 427.1280, found: 427.1290. ¹H NMR (300 MHz, DMSO-d₆) δ ppm 1.49 (q, 2H), 1.88 (t, 2H), 2.15 (qd, 2H), 4.29 (dq, 2H), 6.49 (dd, 2H), 7.00 (d, 1H), 7.34-7.50 (m, 2H), 7.71 (d, 1H), 7.83 (dd, 1H), 7.97 (d, 1H), 8.02 (d, 1H), 8.11 (d, 1H).

### Example 34

### 1-(6-(((1S,3S)-3-((5-Chloropyrazin-2-yl)amino)cyclopentyl)amino)pyridin-3-yl)-1,8-naphthyridin-2 (1H)-one—compound 34

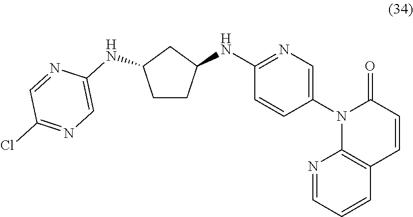 [SMILES: O=c1ccc2cccnc2n1-c1ccc(N[C@H]2CC[C@H](Nc3cnc(Cl)cn3)C2)nc1]

According to GM2 1-(6-(((1S,3S)-3-aminocyclopentyl)amino)pyridin-3-yl)-1,8-naphthyridin-2 (1H)-one compound i-20b (120 mg, 0.34 mmol), 2,5-dichloropyrazine (50 mg, 0.34 mmol), Cs₂CO₃ (328 mg, 1.01 mmol) and Pd-PEPPSI-IpentCl 2-methylpyridine (14 mg, 0.02 mmol) were reacted in 1,4-dioxane (10 mL) 100° C. for 15 h to give upon aqueous work-up and purification by preparative TLC (MeOH:DCM=1:25, 230 mg material isolated) followed by preparative HPLC (PrepMethod B, gradient: 30-48%) the title compound (15 mg, 10%) as a white solid. HRMS (ESI) m/z [M+H]⁺ calcd for C₂₂H₂₁ClN₇O: 434.1490, found: 434.1452. ¹H NMR (300 MHz, DMSO-d₆) δ ppm 1.44-1.64 (2H, m), 1.80-2.01 (2H, m), 2.11-2.24 (2H, m), 4.26 (1H, s), 4.36 (1H, s), 6.58 (1H, d), 6.76 (1H, d), 6.81-6.92 (1H, m), 7.23-7.33 (2H, m), 7.44 (d, 1H) 7.74 (1H, d), 7.79 (1H, d), 8.02-8.06 (2H, m), 8.21 (1H, dd), 8.44 (1H, dd).

### Example 35

### 1-(6-(((1S,3S)-3-((5-Chloropyrazin-2-yl)amino)cyclopentyl)amino)pyridin-3-yl)quinolin-2 (1H)-one—compound 35

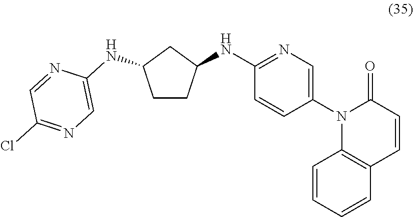 [SMILES: O=c1ccc2ccccc2n1-c1ccc(N[C@H]2CC[C@H](Nc3cnc(Cl)cn3)C2)nc1]

Prepared in analogy to Example 34 from 1-(6-(((1S,3S)-3-aminocyclopentyl)amino)pyridin-3-yl)quinolin-2 (1H)-one compound i-21c (80 mg, 0.22 mmol) and 2,5-dichloropyrazine (50 mg, 0.34 mmol) and purified by preparative TLC (7 M NH₃ in MeOH:DCM=1:25, 80 mg material isolated) followed by preparative HPLC (PrepMethod B, gradient: 42-62%) to give a white solid (28 mg, 29%). ¹H NMR (300 MHz, DMSO-d₆) δ ppm 1.46-1.63 (2H, m), 1.94 (2H, t), 2.08-2.25 (2H, m), 4.23-4.44 (2H, m), 6.63-6.69 (2H, m), 6.74 (1H, d), 7.04 (1H, d), 7.24 (1H, t), 7.31 (1H, dd), 7.41-7.49 (2H, m), 7.72-7.78 (2H, m), 7.85 (1H, d), 8.01 (1H, d), 8.05 (1H, d). HRMS (ESI) m/z [M+H]⁺ calcd for C₂₃H₂₂ClN₆O: 433.1538, found: 433.1500.

### Example 36

### 3-Methoxy-6′-(((1S,3S)-3-((5-methylpyrazin-2-yl)amino)cyclopentyl)amino)-2H-[1,3′-bipyridin]-2-one—compound 36

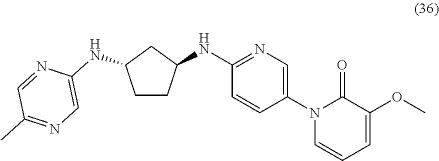

According to GM2 6′-chloro-3-methoxy-2H-[1,3′-bipyridin]-2-one compound i-25a (150 mg, 0.63 mmol) was added to a mixture of (1S,3S)—N¹-(5-methylpyrazin-2-yl)cyclopentane-1,3-diamine compound i-31b (1.39 g, 1.27 mmol), Cs₂CO₃ (1.65 g, 5.07 mmol) and Pd-PEPPSI-IpentCl 2-methylpyridine (27 mg, 0.03 mmol) in 1,4-dioxane (15 mL) at 20° C. and the mixture was stirred at 100° C. for 15 h under nitrogen. The reaction mixture was filtered and the solvent removed under reduced pressure. The residue was purified by preparative TLC (MeOH (7 M NH₃):DCM=1:20) and the obtained material further purified using prep. HPLC (PrepMethod B, gradient: 17-37%) to afford the title compound (87 mg, 35%) as a white solid. HRMS (ESI) m/z [M+H]⁺ calcd for C₂₁H₂₅N₆O₂: 393.2034, found: 393.2030. ¹H NMR (300 MHz, DMSO-d₆) δ ppm 1.50 (2H, tt), 1.89 (2H, t), 2.29-2.08 (5H, m), 3.73 (3H, s), 4.29 (2H, ddd), 6.21 (1H, t), 6.56 (1H, d), 6.93-6.83 (2H, in), 7.04 (1H, s), 7.17 (1H, dd), 7.41 (1H, d), 7.82 (2H, t), 7.92 (1H, d).

TABLE 5
The following Examples (37-42) where prepared in analogy to Example 36 starting from compound i-31b and the given chloro-
pyridine intermediate, applying Buchwald-Hartwig amination (GM2).

| | | | Purification | |
|---|---|---|---|---|
| | | | Method/ | Inter- |
| Example | Structure/Name | Analytical Data | Yield | mediate |
| 37 | 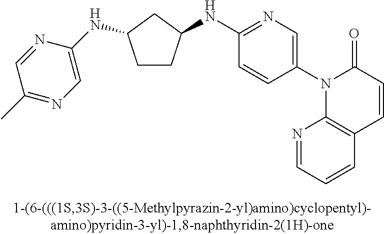 [SMILES: Cc1cnc(N[C@H]2CC[C@H](Nc3ccc(-n4c(=O)ccc5cccnc54)cn3)C2)cn1] | HRMS (ESI) m/z [M + H]⁺ calcd for C₂₃H₂₄N₇O: 414.2036, found: 414.1998. ¹H NMR (300 MHz, DMSO-d₆) δ ppm 1.42-1.61 (2H, m), 1.90 (2H, m), 2.25 (5H, s), 4.31 (2H, m), 6.58 (1H, d), 6.76 (1H, d), 6.87 (2H, m), 7.28 (2H, m), 7.76-7.86 (3H, m), 8.04 (1H, d), 8.21 (1H, m), 8.44 (1H, m). | Prep. TLC (MeOH (7 M NH₃): DCM = 1:20) and prep. HPLC applying PrepMethod B (gradient: 23-43%); (84 mg, 42%) | com- pound i-6a |
| 38 | 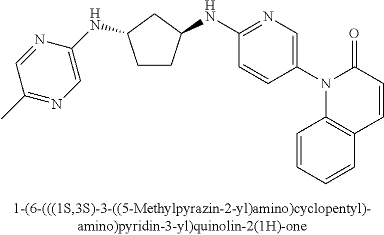 [SMILES: Cc1cnc(N[C@H]2CC[C@H](Nc3ccc(-n4c(=O)ccc5ccccc54)cn3)C2)cn1] | HRMS (ESI) m/z [M + H]⁺ calcd for C₂₄H₂₅N₆O: 413.2084, found: 413.2054. ¹H NMR (300 MHz, DMSO-d₆) δ ppm 1.43-1.63 (2H, m), 1.86-1.97 (2H, m), 2.10-2.29 (5H, m), 4.21- 4.44 (2H, m), 6.66 (2H, m), 6.74 (1H, d), 6.89 (1H, d), 7.02 (1H, d), 7.20-7.34 (2H, m), 7.46 (1H, m), 7.76 (1H, m), 7.80-7.87 (3H, m), 8.01 (1H, d). | Prep. TLC (MeOH (7 M NH₃): DCM = 1:20) and prep. HPLC applying PrepMethod B (gradient: 27-57%); (60 mg, 42%) | com- pound i-21a |
| 39 | 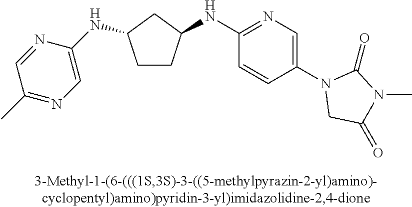 | HRMS (ESI) m/z [M + H]⁺ calcd for C₁₉H₂₄N₇O₂: 382.1986, found: 382.1950. ¹H NMR (300 MHz, DMSO-d₆) δ ppm 1.54-1.39 (2H, m), 1.85 (2H, t), 2.13 (2H, qd), 2.24 (3H, s), 2.92 (3H, s), 4.31-4.17 (2H, m), 4.38 (2H, s), 6.49 (1H, d), 6.62 (1H, d), 6.85 (1H, d), 7.63 (1H, dd), 7.87-7.78 (2H, m), 8.10 (1H, d). | Prep. TLC MeOH (7 M NH₃): DCM = 1:15) and prep. HPLC applying PrepMethod C (gradient: 12-40%); (43 mg, 25%) | com- pound i-23a |
| 40 | 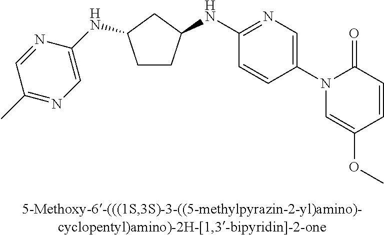 | HRMS (ESI) m/z [M + H]⁺ calcd for C₂₁H₂₅N₆O₂: 393.2034, found: 393.2022. ¹H NMR (300 MHz, DMSO-d₆) δ ppm 1.35-1.63 (2H, m), 1.86 (2H, t), 2.03- 2.17 (2H, m), 2.22 (3H, s), 3.63 (3H, s), 4.28 (2H, dq), 6.41 (1H, d), 6.51 (1H, d), 6.87 (2H, dd), 7.17 (1H, d), 7.37 (2H, ddd), 7.68-7.88 (2H, m), 7.95 (1H, d). | Prep. TLC (MeOH (7 M NH₃): DCM = 1:20) and prep. HPLC applying PrepMethod B (gradient: 22-42%); (65 mg, 26%) | com- pound i-26a |
| 41 | 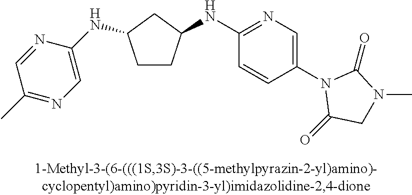 | HRMS (ESI) m/z [M + H]⁺ calcd for C₁₉H₂₄N₇O₂: 382.1986, found: 382.1966. ¹H NMR (300 MHz, DMSO-d₆) δ ppm 1.39- 1.53 (2H, m), 1.85 (2H, m), 2.06-2.18 (2H, m), 2.22 (3H, s), 2.89 (3H, s), 4.06 (2H, s), 4.26 (2H, m), 6.49 (1H, d), 6.85 (2H, m), 7.25 (1H, m), 7.76-7.85 (3H, m). | Prep. TLC (MeOH (7 M NH₃): DCM = 1:20) and prep. HPLC applying PrepMethod B (gradient: 24-39%); (20 mg, 34%) | com- pound i-7b |
| 42 | 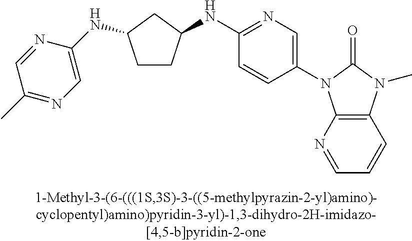 | HRMS (ESI) m/z [M + H]⁺ calcd for C₂₂H₂₅N₈O: 417.2146, found: 417.2176. ¹H NMR (300 MHz, DMSO-d₆) δ ppm 1.40-1.57 (2H, m), 1.88 (2H, t), 2.10- 2.19 (2H, m), 2.22 (3H, s), 3.39 (3H, s), 4.29 (2H, dq), 6.57 (1H, d), 6.88 (2H, dd), 7.12 (1H, dd), 7.44-7.60 (2H, m), 7.80 (2H, q), 7.92 (1H, dd), 8.07 (1H, d). | Prep. TLC (MeOH (7 M NH₃): DCM = 1:20) and prep. HPLC applying PrepMethod C (gradient: 18-38%); (45 mg, 28%) | com- pound i-24a |

### Example 43

### 5-Chloro-6′-(((1S,3S)-3-((5-methylpyrazin-2-yl)amino)cyclopentyl)amino)-2H-[1,3′-bipyridin]-2-one—compound 43

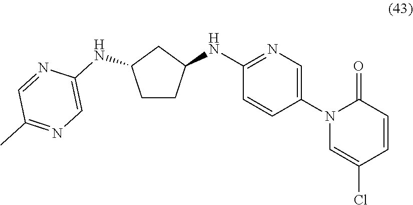 [SMILES: Cc1cnc(N[C@H]2CC[C@H](Nc3ccc(-n4cc(Cl)ccc4=O)cn3)C2)cn1]

According to GM3 (1S,3S)—N¹-(5-iodopyridin-2-yl)-N3-(5-methylpyrazin-2-yl)cyclopentane-1,3-diamine compound i-32a (100 mg, 0.25 mmol), 5-chloropyridin-2-ol (CAS Reg. No. 4214-79-3) (98 mg, 0.76 mmol), Cs₂CO₃ (247 mg, 0.76 mmol), Cu(I)I (48 mg, 0.25 mmol) and rel-(1R,2R)—N¹,N²-dimethylcyclohexane-1,2-diamine (36 mg, 0.25 mmol) were reacted in 1,4-dioxane (3 mL) at 100° C. for 15 h to give upon aqueous work-up and purification by preparative TLC (MeOH (7 M NH₃):DCM=1:20) followed by preparative HPLC (PrepMethod B, gradient: 31-48%) the title compound (31 mg, 31%) as a white solid. HRMS (ESI) m/z [M+H]⁺ calcd for C₂₀H₂₂ClN₆O: 397.1538, found: 397.1512. ¹H NMR (300 MHz, DMSO-d₆) δ ppm 1.31-1.71 (2H, m), 1.85 (2H, t), 2.11 (2H, ddd), 2.22 (3H, s), 4.27 (2H, dq), 6.34-6.60 (2H, m), 6.86 (1H, d), 6.97 (1H, d), 7.39 (1H, dd), 7.53 (1H, dd), 7.72-7.84 (2H, m), 7.84-7.98 (2H, m).

TABLE 6
The following Examples (44-46) where prepared in analogy to Example 43 starting from compound i-32a and the given nucleophiles,
applying Ullmann coupling (GM3). For the preparation of Example 46 Cs₂CO₃ was substituted by K₂CO₃.

| | | | Purification | Nucleophile |
|---|---|---|---|---|
| | | | Method/ | (CAS |
| Example | Structure/Name | Analytical Data | Yield | Reg. No.) |
| 44 | 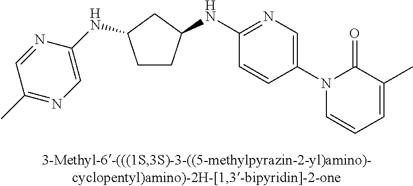 | HRMS (ESI) m/z [M + H]⁺ calcd for C₂₁H₂₅N₆O: 377.2084, found: 377.2080. ¹H NMR (300 MHz, DMSO-d₆) δ ppm 1.38-1.56 (2H, m), 1.86 (2H, t), 2.00 (3H, s), 2.05-2.20 (2H, m), 2.22 (3H, s), 4.28 (2H, dq), 6.18 (1H, t), 6.50 (1H, d), 6.88 (2H, dd), 7.22-7.51 (3H, m), 7.73-7.99 (3H, m). | Prep. TLC (MeOH (7 M NH₃): DCM = 1:20) and prep. HPLC applying PrepMethod B (gradient: 19-49%); (45 mg, 47%) | 1003-56-1 |
| 45 | 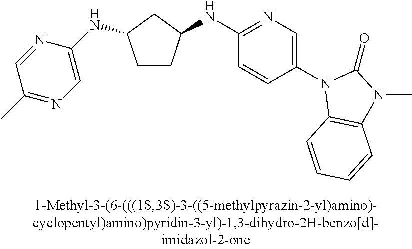 | HRMS (ESI) m/z [M + H]⁺ calcd for C₂₃H₂₆N₇O: 416.2194, found: 416.2198. ¹H NMR (300 MHz, DMSO-d₆) δ ppm 1.62-1.37 (2H, m), 1.90 (2H, t), 2.21- 2.07 (2H, m), 2.25 (s, 3H), 3.39 (s, 3H), 4.31 (2H, dq), 6.61 (1H, dd), 6.91-6.85 (2H, m), 6.98 (1H, d), 7.04 (1H, td), 7.12 (1H, td), 7.26-7.19 (1H, m), 7.46 (1H, dd), 7.82 (2H, q), 8.05 (1H, d). | Prep. TLC (MeOH (7 M NH₃): DCM = 1:20) and prep. HPLC applying PrepMethod B (gradient: 30-50%); (57 mg, 54%) | 1849-01-0 |
| 46 | 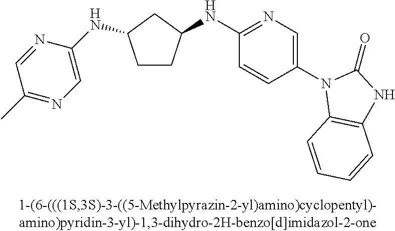 [SMILES: Cc1cnc(N[C@H]2CC[C@H](Nc3ccc(-n4c(=O)[nH]c5ccccc54)cn3)C2)cn1] | HRMS (ESI) m/z [M + H]⁺ calcd for C₂₂H₂₄N₇O: 402.2036, found: 402.2040. ¹H NMR (300 MHz, DMSO-d₆) δ ppm 1.62-1.41 (2H, m), 1.90 (2H, t), 2.22- 2.07 (2H, m), 4.31 (2H, dq), 6.60 (1H, d), 6.85 (2H, dd), 7.01-6.92 (2H, m), 7.08- 7.01 (2H, m), 7.46 (1H, dd), 7.90-7.75 (m, 2H), 8.04 (1H, d), 11.05 (s, 1H). | Prep. TLC (MeOH (7 M NH₃): DCM = 1:20) and prep. HPLC applying PrepMethod B (gradient: 24-44%); (40 mg, 39%) | 615-16-7 (K₂CO₃ used as base) |

### Example 47

### 3-Chloro-6′-(((1S,3S)-3-((5-methylpyrazin-2-yl)amino)cyclopentyl)amino)-2H-[1,3′-bipyridin]-2-one—compound 47

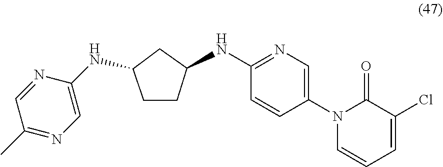 [SMILES: Cc1cnc(N[C@H]2CC[C@H](Nc3ccc(-n4cccc(Cl)c4=O)cn3)C2)cn1]

According to GM3 (1S,3S)—N¹-(5-iodopyridin-2-yl)-NM-(5-methylpyrazin-2-yl)cyclopentane-1,3-diamine compound i-32a (100 mg, 0.25 mmol) was added to 3-chloropyridin-2 (1H)-one (CAS Reg. No. 13466-35-8) (131 mg, 1.01 mmol), Cs₂CO₃ (247 mg, 0.76 mmol), rel-(1R,2R)—N¹,N¹-dimethylcyclohexane-1,2-diamine (36 mg, 0.25 mmol) and Cu(I)I (48 mg, 0.25 mmol) in 1,4-dioxane (30 mL) at 20° C. and the resulting mixture was stirred at 100° C. for 15 h under nitrogen. The reaction mixture was poured into water (125 mL) and the aqueous layer was extracted with EtOAc (6×100 mL). The combined organic layers were dried over Na₂SO₄, filtered and evaporated. The obtained residue was purified by preparative TLC (MeOH (7 M NH₃):DCM=1:20) followed by preparative HPLC (PrepMethod E, gradient: 5-23%) to afford the title compound (43 mg, 43%) as a white solid. HRMS (ESI) m/z [M+H]⁺ calcd for C₂₀H₂₂ClN₆O: 397.1538, found: 397.1518. ¹H NMR (300 MHz, DMSO-d₆) δ 1.40-1.54 (2H, m), 1.86 (2H, t), 2.08-2.19 (2H, m), 2.22 (3H, s), 4.28 (2H, dq), 6.29 (1H, t), 6.52 (1H, d), 6.86 (1H, d), 7.00 (1H, d), 7.42 (1H, dd), 7.65 (1H, dd), 7.79-7.81 (3H, m), 7.95 (1H, d).

### Example 48

### 6′-(((1S,3S)-3-((5,6-Dimethylpyrazin-2-yl)amino)cyclopentyl)amino)-2-oxo-2H-[1,3′-bipyridine]-5-carbonitrile—compound 48

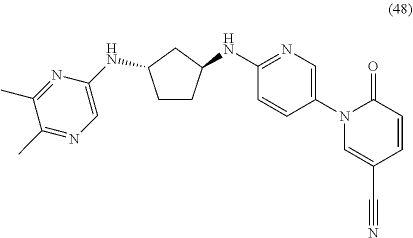 [SMILES: Cc1ncc(N[C@H]2CC[C@H](Nc3ccc(-n4cc(C#N)ccc4=O)cn3)C2)nc1C]

According to GM2 6′-(((1S,3S)-3-aminocyclopentyl)amino)-2-oxo-2H-[1,3′-bipyridine]-5-carbonitrile compound i-33b (50 mg, 0.15 mmol), 5-chloro-2,3-dimethylpyrazine (CAS Reg. No. 59489-32-6) (43 mg, 0.30 mmol), Cs₂CO₃ (147 mg, 0.45 mmol) and Pd-PEPPSI-IpentCl 2-methylpyridine (6.3 mg, 7.5 μmol) were reacted in 1,4-dioxane (6 mL) at 100° C. for 15 h to give upon non-aqueous work-up and purification by preparative TLC (MeOH:DCM=1:10) followed by preparative HPLC (PrepMethod E, gradient: 5-24%) the title compound (5 mg, 8%) as a white solid. HRMS (ESI) m/z [M+H]⁺ calcd for C₂₂H₂₄N₇O: 402.2036, found: 402.2036. ¹H NMR (300 MHz, Methanol-d₄) δ ppm 1.48-1.68 (2H, m), 1.99 (2H, t), 2.26 (2H, m), 2.32 (6H, s), 4.35 (2H, p), 6.62 (2H, t), 7.45 (1H, m), 7.56 (1H, s), 7.69 (1H, m), 7.97 (1H, d), 8.35 (1H, d).

### Example 49

### 6′-(((1S,3S)-3-((5-Methylpyrazin-2-yl)amino)cyclopentyl)amino)-2-oxo-2H-[1,3′-bipyridine]-5-carbonitrile—compound 49

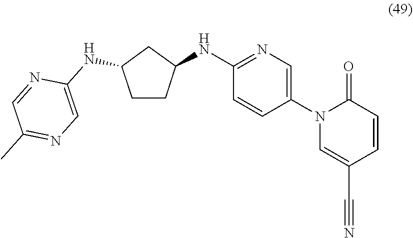 [SMILES: Cc1cnc(N[C@H]2CC[C@H](Nc3ccc(-n4cc(C#N)ccc4=O)cn3)C2)cn1]

Prepared in analogy to Example 48 from 6′-(((1S,3S)-3-aminocyclopentyl)amino)-2-oxo-2H-[1,3′-bipyridine]-5-carbonitrile compound i-33b (50 mg, 0.15 mmol) and 2-bromo-5-methylpyrazine (CAS Reg. No. 98006-90-7) (52 mg, 0.30 mmol) and purified by preparative TLC (MeOH:DCM=1:10) followed by preparative HPLC (PrepMethod E, gradient: 4-22%) to give a white solid (5 mg, 9%). HRMS (ESI) m/z [M+H]⁺ calcd for C₂₁H₂₂N₇O: 388.1880, found: 388.1884. ¹H NMR (300 MHz, Methanol-d₄) δ ppm 1.54-1.66 (2H, m), 1.99 (2H, t), 2.27 (2H, td), 2.32 (3H, s), 4.33 (2H, dt), 6.55-6.69 (2H, m), 7.45 (1H, dd), 7.69 (1H, dd), 7.80 (1H, s), 7.83 (1H, s), 7.97 (1H, d), 8.35 (1H, dd).

### Example 77-80

TABLE 9
The following Examples (77-80) where prepared from compound i-19d and the given nucleophiles, applying Ullmann coupling (GM3A).

| | | | Purification | Nucleophile |
|---|---|---|---|---|
| | | | Method/ | (CAS |
| Ex | Structure/Name | Analytical Data | Yield | Reg. No.) |
| 77 | 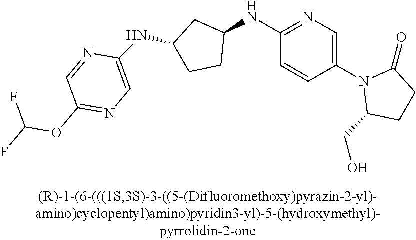 [SMILES: O=C1CC[C@H](CO)N1c1ccc(N[C@H]2CC[C@H](Nc3cnc(OC(F)F)cn3)C2)nc1] | HRMS (ESI) m/z [M + H]⁺ calcd for C₂₀H₂₅F₂N₆O₃: 435.1950, found: 435.1938. | preparative HPLC applying PrepMethod Parallel A; (1.9 mg, 17%) | 66673-40-3 |
| 78 | 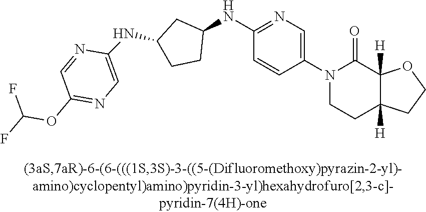 [SMILES: O=C1[C@@H]2OCC[C@@H]2CCN1c1ccc(N[C@H]2CC[C@H](Nc3cnc(OC(F)F)cn3)C2)nc1] | HRMS (ESI) m/z [M + H]⁺ calcd for C₂₂H₂₇F₂N₆O₃: 461.2106, found: 461.2088. ¹H NMR (600 MHz, DMSO-d₆) δ ppm 1.40-1.45 (2H, m), 1.56-1.67 (2H, m), 1.79-1.81 (2H, m), 1.87- 1.92 (1H, m), 2.05-2.11 (4H, m), 3.40-3.44 (1H, m), 3.51-3.55 (1H, m), 3.60- 3.64 (1H, m), 3.76-3.80 (1H, m), 4.07 (1H, d), 4.13- 4.24 (2H, m), 7.08 (1H, d), | preparative HPLC applying PrepMethod Parallel A; (3.9 mg, 34%) | 1823269- 15-3 |
| | | 7.21 (1H, dd), 7.26 (1H, t), | | |
| | | 7.54 (1H, d), 7.79 (1H, d), | | |
| | | 7.85 (1H, d). | | |
| 79 | 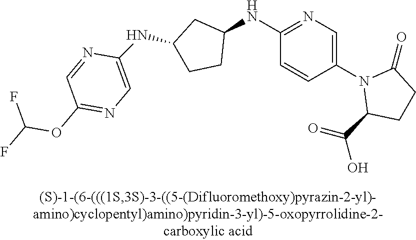 [SMILES: O=C(O)[C@@H]1CCC(=O)N1c1ccc(N[C@H]2CC[C@H](Nc3cnc(OC(F)F)cn3)C2)nc1] | HRMS (ESI) m/z [M + H]⁺ calcd for C₂₀H₂₃F₂N₆O₄: 449.1744, found: 449.1722. ¹H NMR (600 MHz, DMSO-d₆) δ ppm 1.34-1.43 (2H, m), 1.74-1.81 (2H, m), 2.04-2.08 (2H, m), 4.10- 4.22 (2H, m), 6.31-6.42 (2H, m), 7.05 (1H, d), 7.26 (1H, t), 7.53 (1H, s), 7.83 (1H, s). | preparative HPLC applying PrepMethod Parallel A; (6.4 mg, 57%) | 98-79-3 |
| 80 | 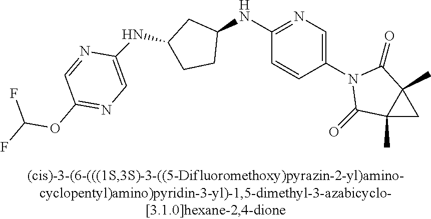 [SMILES: C[C@@]12C[C@]1(C)C(=O)N(c1ccc(N[C@H]3CC[C@H](Nc4cnc(OC(F)F)cn4)C3)nc1)C2=O] | HRMS (ESI) m/z [M + H]⁺ calcd for C₂₂H₂₅F₂N₆O₃: 459.1950, found: 459.1924. ¹H NMR (600 MHz, DMSO-d₆) δ ppm 1.18-1.23 (2H, m), 1.32 (6H, s), 1.37- 1.46 (2H, m), 1.79-1.84 (2H, m), 2.06-2.10 (2H, m), 6.42 (1H, d), 6.82 (1H, d), 7.09-7.10 (1H, m), 7.13- 7.39 (2H, m), 7.54 (1H, d), 7.71 (1H, d), 7.85 (1H, d). | preparative HPLC applying PrepMethod Parallel B | 163151- 91-5 (see also JACS 1976, 98, 8144- 8146) |

### Example 103

### 6′-(((1S,3S)-3-((5-Methylpyrazin-2-yl)amino)cyclopentyl)amino)-2-oxo-2H-[1,3′-bipyridine]-3-carbonitrile—compound 103

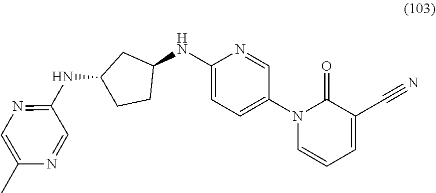 [SMILES: Cc1cnc(N[C@H]2CC[C@H](Nc3ccc(-n4cccc(C#N)c4=O)cn3)C2)cn1]

In a slight variation of GM2 Cs₂CO₃ (450 mg, 1.38 mmol) was added to a mixture of 6′-(((1S,3S)-3-aminocyclopentyl)amino)-2-oxo-2H-[1,3′-bipyridine]-3-carbonitrile 4HCl compound i-35b (122 mg, 0.28 mmol), 2-bromo-5-methylpyrazine (CAS Reg. No. 98006-90-7) (96 mg, 0.55 mmol) and Pd-PEPPSI-IpentCl 2-methylpyridine (12 mg, 0.01 mmol) in DMF (5 mL) at 20° C. and the resulting mixture stirred at 100° C. for 15 h under nitrogen. The reaction mixture was diluted with water (75 mL) and extracted with EtOAc (4×50 mL). The combined organic layers were washed with saturated brine (5×100 mL), dried (Na₂SO₄), filtered and evaporated. The obtained material was purified by preparative TLC (EtOAc, 53 mg material isolated) followed by preparative HPLC (PrepMethod B, gradient: 20-38%) to give the title compound as a yellow solid (28 mg, 26%). HRMS (ESI) m/z [M+H]⁺ calcd for C₂₁H₂₂N₇O: 388.1880, found: 388.1864. ¹H NMR (300 MHz, DMSO-d₆) δ ppm 1.42-1.52 (2H, m), 1.84-1.88 (2H, m), 2.08-2.17 (2H, m), 2.22 (3H, s), 4.18-4.35 (2H, m), 6.45 (1H, t), 6.53 (1H, d), 6.86 (1H, d), 7.05 (1H, d), 7.44 (1H, dd), 7.80-7.81 (2H, m), 7.97 (1H, dd), 8.04 (1H, dd), 8.21 (1H, dd).

### Example 82

### 1-(6-(((1S,3S)-3-((5-(Difluoromethoxy)pyrazin-2-yl)amino)cyclopentyl)amino)pyridin-3-yl)-3-methyl-1,3-dihydro-2H-benzo[d]imidazol-2-one—compound 82

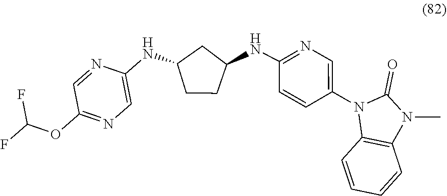 [SMILES: Cn1c(=O)n(-c2ccc(N[C@H]3CC[C@H](Nc4cnc(OC(F)F)cn4)C3)nc2)c2ccccc21]

According to GM3 rel-(1R,2R)—N¹,N²-dimethylcyclohexane-1,2-diamine (CAS Reg. No. 67579-81-1) (95 mg, 0.67 mmol) and Cu(I)I (0.13 g, 0.67 mmol) were added to (1S,3S)—N¹-(5-(difluoromethoxy)pyrazin-2-yl)-N3-(5-iodopyridin-2-yl)cyclopentane-1,3-diamine compound i-19d (100 mg, 0.22 mmol), Cs₂CO₃ (0.22 g, 0.67 mmol) and 1-methyl-1,3-dihydro-2H-benzo[d]imidazol-2-one (CAS Reg. No. 1849-01-0) (99 mg, 0.67 mmol) in 1,4-dioxane (1 mL) at rt. The resulting suspension was stirred at 100° C. for 2 h under a nitrogen atmosphere. The reaction mixture was diluted with EtOAc (100 mL) and washed sequentially with water (3×35 mL) and saturated brine (3×35 mL). The organic layer was dried over Na₂SO₄, filtered and evaporated and the crude material was purified by preparative TLC (EtOAc) followed by preparative HPLC (PrepMethod B, gradient: 41-71%) to give (47 mg, 45%) of the title compound as a white solid. MS (ESI): m/z [M+H]⁺ 468.1. ¹H NMR (300 MHz, DMSO-d₆) δ ppm 1.46-1.58 (2H, m), 1.90 (2H, t), 2.12-2.22 (2H, m), 3.38 (3H, s), 4.21-4.40 (2H, m), 6.60 (1H, dd), 6.87 (1H, dd), 6.98 (1H, d), 7.03 (1H, dt), 7.08-7.57 (5H, m), 7.61 (1H, d), 7.91 (1H, d), 8.04 (1H, d). ¹⁹F NMR (282 MHz, DMSO-d₆) δ ppm −83.75 (2F, s).

### Example 101

### 6′-(((1S,3S)-3-((5-Chloropyrazin-2-yl)amino)cyclopentyl)amino)-2-oxo-2H-[1,3′-bipyridine]-3-carbonitrile—compound 101

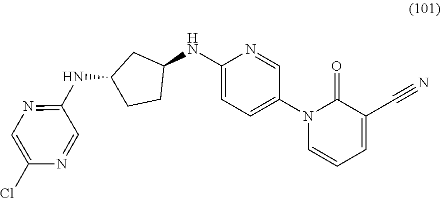 [SMILES: N#Cc1cccn(-c2ccc(N[C@H]3CC[C@H](Nc4cnc(Cl)cn4)C3)nc2)c1=O]

In a slight variation of GM2 Cs₂CO₃ (450 mg, 1.38 mmol) was added to a mixture of 6′-(((1S,3S)-3-aminocyclopentyl)amino)-2-oxo-2H-[1,3′-bipyridine]-3-carbonitrile 4HCl compound i-35b (122 mg, 0.28 mmol), 2,5-dichloropyrazine (82 mg, 0.55 mmol) and Pd-PEPPSI-IpentCl 2-methylpyridine (12 mg, 0.01 mmol) in DMF (5 mL) at 20° C. and the resulting mixture stirred at 100° C. for 15 h under nitrogen. The reaction mixture was diluted with water (100 mL) and extracted with EtOAc (4×75 mL). The combined organic layers were washed with saturated brine (5×100 mL), dried (Na₂SO₄), filtered and evaporated. The obtained material was purified by preparative TLC (EtOAc, 35 mg material isolated) followed by preparative HPLC (PrepMethod B, gradient: 39-54%) to give the title compound as a yellow solid (10 mg, 9%). HRMS (ESI) m/z[M+H]⁺ calcd for C₂₀H₁₉ClN₇O: 408.1334, found: 408.1330. ¹H NMR (300 MHz, DMSO-d₆) δ ppm 1.46-1.56 (2H, m), 1.89 (2H, t), 2.10-2.21 (2H, m), 4.21-4.38 (2H, m), 6.46 (1H, t), 6.53 (1H, d), 7.08 (1H, d), 7.42 (1H, d), 7.46 (1H, dd), 7.72 (1H, d), 7.98 (1H, d), 8.03-8.06 (2H, m), 8.22 (1H, dd).

### Example 81

### 6′-(((1S,3S)-3-((5-(Difluoromethoxy)pyrazin-2-yl)amino)cyclopentyl)amino)-5-(4-methylpiperazin-1-yl)-2H-[1,3′-bipyridin]-2-one—compound 81

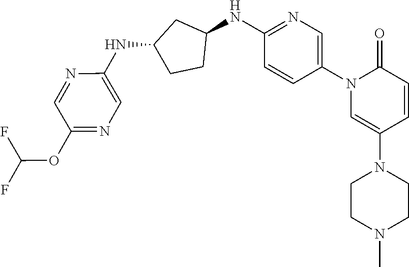 [SMILES: CN1CCN(c2ccc(=O)n(-c3ccc(N[C@H]4CC[C@H](Nc5cnc(OC(F)F)cn5)C4)nc3)c2)CC1]

According to general procedure GM3 (1S,3S)—N¹-(5-(difluoromethoxy)pyrazin-2-yl)-N3-(5-iodopyridin-2-yl)cyclopentane-1,3-diamine compound i-19d (90 mg, 0.20 mmol), 5-(4-methylpiperazin-1-yl)pyridin-2-ol compound i-101b (156 mg, 0.80 mmol), Cs₂CO₃ (197 mg, 0.06 mmol), Cu(I)I (115 mg, 0.60 mmol) and rel-(1R,2R)—N¹,N²-dimethylcyclohexane-1,2-diamine (86 mg, 0.60 mmol) were reacted in 1,4-dioxane (5 mL) at 100° C. for 3 h. Upon aqueous workup (EtOAc) followed by preparative TLC (EtOAc) and preparative HPLC (PrepMethod B, gradient: 23-45%) the title compound (32 mg, 31%) was obtained as a yellow solid. MS (ESI): m/z [M+H]⁺ 513.3. ¹H NMR (300 MHz, DMSO-d₆) δ ppm 1.43-1.56 (2H, m), 1.88 (2H, t), 2.10-2.19 (5H, m), 2.39-2.42 (4H, m), 2.85-2.88 (4H, m), 4.18-4.28 (1H, m), 4.28-4.38 (1H, m), 6.41 (1H, d), 6.51 (1H, d), 6.84 (1H, d), 6.90 (1H, d), 7.08-7.57 (4H, m), 7.60 (1H, d), 7.91 (1H, d), 7.95 (1H, brd). ¹⁹F NMR (282 MHz, DMSO-d₆) δ ppm −83.76 (2F, s).

### Example 83

### 5-Chloro-6′-(((1S,3S)-3-((5-(difluoromethoxy)pyrazin-2-yl)amino)cyclopentyl)amino)-2H-[1,3′-bipyridin]-2-one—compound 83

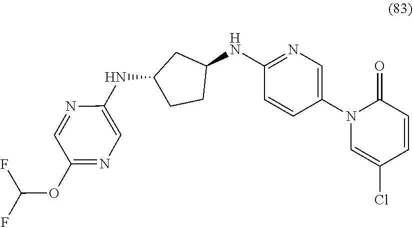 [SMILES: O=c1ccc(Cl)cn1-c1ccc(N[C@H]2CC[C@H](Nc3cnc(OC(F)F)cn3)C2)nc1]

rel-(1R,2R)—N¹,N²-Dimethylcyclohexane-1,2-diamine (31.8 mg, 0.22 mmol) was added to Cs₂CO₃ (219 mg, 0.67 mmol), 5-chloropyridin-2-ol (CAS Reg. No. 4214-79-3) (87 mg, 0.67 mmol), (1S,3S)—N-(5-(difluoromethoxy)pyrazin-2-yl)-N3-(5-iodopyridin-2-yl)cyclopentane-1,3-diamine compound i-19d (100 mg, 0.22 mmol) and Cu(I)I (42.6 mg, 0.22 mmol) in 1,4-dioxane (5 mL) at 20° C. The resulting mixture was stirred at 100° C. for 15 h under a nitrogen atmosphere. The reaction mixture was diluted with EtOAc (75 mL) and washed sequentially with water (3×25 mL) and sat. brine (3×20 mL). The organic layer was dried over Na₂SO₄, filtered and evaporated and the residue purified by preparative TLC (EtOAc) followed by preparative HPLC (PrepMethod M, gradient: 19-49%) to give the title compound (40 mg, 39%) as a white solid. HRMS (ESI) m/z [M+H]⁺ calcd for C₂₀H₂₀ClF₂N₆O₂: 449.1298, found: 449.1308. ¹H NMR (300 MHz, DMSO-d₆) δ ppm 1.40-1.58 (2H, m), 1.88 (2H, t), 2.08-2.22 (2H, m), 4.16-4.40 (2H, m), 6.48 (1H, d), 6.51 (1H, d), 6.99 (1H, d), 7.15 (1H, d), 7.32 (1H, t), 7.41 (1H, dd), 7.54 (1H, dd), 7.60 (1H, d), 7.88-7.92 (2H, m), 7.95 (1H, d).

### Example 84

### 6′-(((1S,3S)-3-((5-(Difluoromethoxy)pyrazin-2-yl)amino)cyclopentyl)amino)-3-methyl-2H-[1,3′-bipyridin]-2-one—compound 84

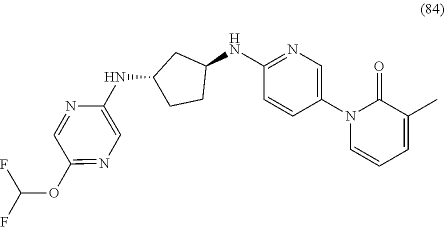 [SMILES: Cc1cccn(-c2ccc(N[C@H]3CC[C@H](Nc4cnc(OC(F)F)cn4)C3)nc2)c1=O]

rel-(1R,2R)—N¹,N²-Dimethylcyclohexane-1,2-diamine (31.8 mg, 0.22 mmol) was added to Cs₂CO₃ (219 mg, 0.67 mmol), 3-methylpyridin-2-ol (CAS Reg. No. 1003-56-1) (73 mg, 0.67 mmol), (1S,3S)—N¹-(5-(difluoromethoxy)pyrazin-2-yl)-N3-(5-iodopyridin-2-yl)cyclopentane-1,3-diamine compound i-19d (100 mg, 0.22 mmol) and Cu(I)I (43 mg, 0.22 mmol) in 1,4-dioxane (5 mL) at 20° C. The resulting mixture was stirred at 100° C. for 15 h under a nitrogen atmosphere. The reaction mixture was diluted with EtOAc (75 mL) and washed sequentially with water (3×25 mL) and sat. brine (3×20 mL). The organic layer was dried over Na₂SO₄, filtered and evaporated. The residue was purified by preparative TLC (100% EtOAc) followed by preparative HPLC (PrepMethod D, gradient: 30-60%) to give the title compound (58 mg, 60%) as a white solid. HRMS (ESI) m/z [M+H]⁺ calcd for C₂₁H₂₃F₂N₆O₂: 429.1844, found: 429.1810. ¹H NMR (300 MHz, DMSO-d₆) δ ppm 1.41-1.58 (2H, m), 1.88 (2H, t), 2.01 (3H, s), 2.08-2.24 (2H, m), 4.16-4.41 (2H, m), 6.19 (1H, t), 6.52 (1H, d), 6.92 (1H, d), 7.15 (1H, d), 7.32 (1H, t), 7.33-7.42 (2H, m), 7.45 (1H, dd), 7.60 (1H, d), 7.89-7.93 (2H, m).

### Example 85

### 3-Chloro-6′-(((1S,3S)-3-((5-(difluoromethoxy)pyrazin-2-yl)amino)cyclopentyl)amino)-2H-[1,3′-bipyridin]-2-one—compound 85

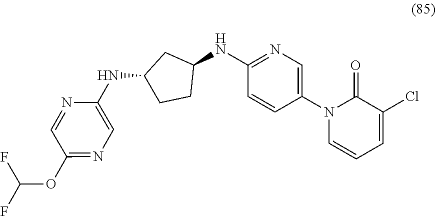 [SMILES: O=c1c(Cl)cccn1-c1ccc(N[C@H]2CC[C@H](Nc3cnc(OC(F)F)cn3)C2)nc1]

(1S,3S)—N¹-(5-(Difluoromethoxy)pyrazin-2-yl)-N3-(5-iodopyridin-2-yl)cyclopentane-1,3-diamine compound i-19d (110 mg, 0.25 mmol) was added to 3-chloropyridin-2-ol (CAS Reg. No. 13466-35-8) (159 mg, 1.23 mmol), rel-(1R,2R)—N¹,N²-dimethylcyclohexane-1,2-diamine (35 mg, 0.25 mmol), Cu(I)I (46.8 mg, 0.25 mmol) and K₂CO₃ (136 mg, 0.98 mmol) in 1,4-dioxane (10 mL) at 20° C. The resulting mixture was stirred at 100° C. for 15 h under a nitrogen atmosphere. The reaction mixture was poured into water (150 mL) and extracted with EtOAc (4×100 mL). The organic layer was dried over Na₂SO₄, filtered and evaporated. The residue was purified by preparative TLC (7 M NH₃ in MeOH:DCM, 1:15) followed by preparative HPLC (PrepMethod B, gradient: 36-56%) to give the title compound (41 mg, 37%) as a white solid.

HRMS (ESI) m/z [M+H]⁺ calcd for C₂₀H₂₀ClF₂N₆O₂: 449.1298, found: 449.1290. ¹H NMR (300 MHz, DMSO-d₆) δ ppm 1.38-1.66 (2H, m), 1.92 (2H, t), 2.10-2.26 (2H, m), 4.34 (2H, br d), 6.33 (1H, t), 6.65 (1H, br d), 7.17 (1H, br s), 7.33 (1H, t), 7.55 (1H, br d), 7.62 (1H, d), 7.67 (1H, dd), 7.84 (1H, dd), 7.92 (1H, d), 8.02 (1H, d).

### Example 86

### 6′-(((1S,3S)-3-((5-(Difluoromethoxy)pyrazin-2-yl)amino)cyclopentyl)amino)-2H-[1,3′-bipyridin]-2-one—compound 86

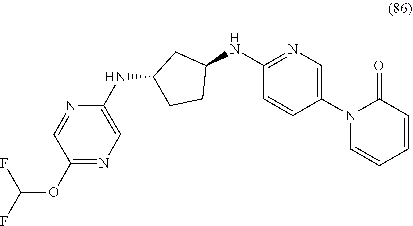 [SMILES: O=c1ccccn1-c1ccc(N[C@H]2CC[C@H](Nc3cnc(OC(F)F)cn3)C2)nc1]

According to general procedure GM2 6′-(((1S,3S)-3-aminocyclopentyl)amino)-2H-[1,3′-bipyridin]-2-one compound i-1c (165 mg, 0.61 mmol), 2-chloro-5-(difluoromethoxy)pyrazine compound i-19a (100 mg, 0.55 mmol), Cs₂CO₃ (541 mg, 1.66 mmol) and Pd PEPPSI-IpentCl 2-methylpyridine (37 mg, 0.04 mmol) were reacted in 1,4-dioxane (5 mL) at 100° C. for 18 h under a nitrogen atmosphere. Upon the alternative aqueous workup (EtOAc) followed by preparative TLC (EtOAc) and preparative HPLC (PrepMethod M, gradient: 29-59%) the title compound (53 mg, 23%) was obtained as a white solid. HRMS (ESI) m/z [M+H]⁺ calcd for C₂₀H₂₁F₂N₆O₂: 415.1688, found: 415.1710. ¹H NMR (300 MHz, DMSO-d₆) δ ppm 1.44-1.58 (2H, m), 1.89 (2H, t), 2.11-2.21 (2H, m), 4.19-4.29 (1H, m), 4.29-4.40 (1H, m), 6.28 (1H, dt), 6.45 (1H, ddd), 6.53 (1H, dd), 6.95 (1H, d), 7.16 (1H, d), 7.33 (1H, t), 7.40 (1H, dd), 7.48 (1H, ddd), 7.59-7.62 (2H, m), 7.91 (1H, d), 7.93 (1H, dd). ¹⁹F NMR NMR (282 MHz, DMSO-d₆) δ ppm −83.76 (2F, s).

### Example 87

### 1-(6-(((1S,3S)-3-((5-(Difluoromethoxy)pyrazin-2-yl)amino)cyclopentyl)amino)pyridin-3-yl)-1,8-naphthyridin-2 (1H)-one—compound 87

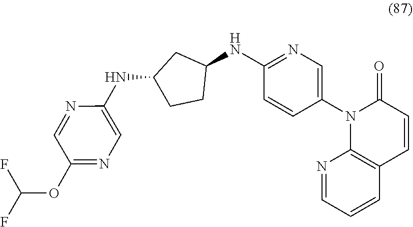 [SMILES: O=c1ccc2cccnc2n1-c1ccc(N[C@H]2CC[C@H](Nc3cnc(OC(F)F)cn3)C2)nc1]

According to general procedure GM2 1-(6-(((1S,3S)-3-aminocyclopentyl)amino)pyridin-3-yl)-1,8-naphthyridin-2 (1H)-one compound i-20b (100 mg, 0.31 mmol), 2-chloro-5-(difluoro-methoxy)pyrazine compound i-19a (56 mg, 0.31 mmol), Cs₂CO₃ (304 mg, 0.93 mmol) and Pd PEPPSI-IpentCl 2-methylpyridine (26 mg, 0.03 mmol) were reacted in 1,4-dioxane (5 mL) at 100° C. for 18 h under a nitrogen atmosphere. Upon the alternative aqueous workup (EtOAc) followed by preparative TLC (MeOH:DCM=1:20) and preparative HPLC (PrepMethod D, gradient: 29-49%) the title compound (25 mg, 17%) was obtained as a white solid. MS (ESI): m/z [M+H]⁺ 466.25. ¹H NMR (300 MHz, DMSO-d₆) δ ppm 1.45-1.59 (2H, m), 1.84-1.98 (2H, m), 2.11-2.24 (2H, m), 4.20-4.30 (1H, m), 4.30-4.41 (1H, m), 6.57 (1H, d), 6.76 (1H, d), 6.85 (1H, d), 7.16 (1H, d), 7.25 (1H, dd), 7.30 (1H, dd), 7.32 (1H, t), 7.61 (1H, d), 7.79 (1H, d), 7.92 (1H, d), 8.04 (1H, d), 8.20 (1H, dd), 8.44 (1H, dd). ¹⁹F NMR (282 MHz, DMSO-d₆) δ ppm −83.73 (2F, s).

### Example 88

### 1-(6-(((1S,3S)-3-((5-(Difluoromethoxy)pyrazin-2-yl)amino)cyclopentyl)amino)pyridin-3-yl)quinolin-2 (1H)-one—compound 88

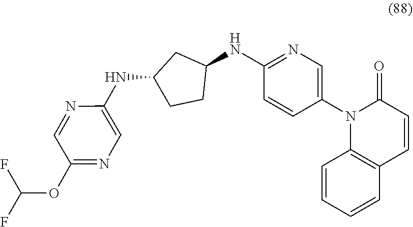 [SMILES: O=c1ccc2ccccc2n1-c1ccc(N[C@H]2CC[C@H](Nc3cnc(OC(F)F)cn3)C2)nc1]

In a slight variation of GM2 1-(6-(((1S,3S)-3-aminocyclopentyl)amino)pyridin-3-yl)quinolin-2 (1) n-one compound i-21c (100 mg, 0.31 mmol), 2-chloro-5-(difluoromethoxy)pyrazine compound i-19a (85 mg, 0.47 mmol), Cs₂CO₃ (305 mg, 0.94 mmol) and Pd PEPPSI-IpentCl 2-methylpyridine (26 mg, 0.03 mmol) were reacted in a mixture of 1,4-dioxane (2 mL) and DMA (1 mL) at 100° C. for 18 h under a nitrogen atmosphere. Upon the alternative aqueous workup (EtOAc) followed by preparative TLC (MeOH:DCM=1:20) and preparative HPLC (PrepMethod C, gradient: 27-64%) the title compound (6 mg, 4%) was obtained as a white solid. HRMS (ESI) m/z [M+H]⁺ calcd for C₂₄H₂₃F₂N₆O₂: 465.1844, found: 465.1852. ¹H NMR (300 MHz, MeOH-d₄) δ ppm 1.56-1.72 (2H, m), 2.04 (2H, dt), 2.24-2.37 (2H, m), 4.29-4.44 (2H, m), 6.72-6.73 (1H, m), 6.76-6.76 (1H, m), 6.89 (1H, d), 7.14 (1H, t), 7.27-7.35 (2H, m), 7.46-7.52 (1H, m), 7.57 (1H, t), 7.74 (1H, dd), 7.80 (1H, t), 7.85 (1H, dd), 8.04 (1H, d). ¹⁹F NMR (282 MHz, MeOH-d₄) δ ppm −87.07 (2F, s).

### Example 89

### 6′-(((1S,3S)-3-((5-(Difluoromethoxy)pyrazin-2-yl)amino)cyclopentyl)amino)-2-oxo-2H-[1,3′-bipyridine]-5-carbonitrile—compound 89

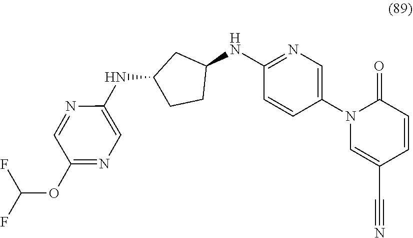 [SMILES: N#Cc1ccc(=O)n(-c2ccc(N[C@H]3CC[C@H](Nc4cnc(OC(F)F)cn4)C3)nc2)c1]

6′-(((1S,3S)-3-Aminocyclopentyl)amino)-2-oxo-2H-[1,3′-bipyridine]-5-carbonitrile HCl compound i-33b (50 mg, 0.15 mmol) was added to Pd-PEPPSI-IpentCl 2-methylpyridine (6.3 mg, 7.5 μmol), 2-chloro-5-(difluoromethoxy)pyrazine compound i-19a (54 mg, 0.30 mmol) and Cs₂CO₃ (147 mg, 0.45 mmol) in 1,4-dioxane (5 mL). The resulting mixture was stirred at 100° C. for 15 h under a nitrogen atmosphere. The reaction mixture was filtered and the filter cake was washed with EtOAc (3×20 mL). The combined filtrates were re-filtered, concentrated under reduced pressure and the residue was purified by preparative TLC (MeOH:DCM, 1:10) followed by preparative HPLC (PrepMethod J, gradient: 15-45%) to give the title compound (13 mg, 19%) as a white solid. HRMS (ESI) m/z [M+H]⁺ calcd for C₂₁H₂₀F₂N₇O₂: 440.1640, found: 440.1628. ¹H NMR (300 MHz, CD₃OD) δ ppm 1.52-1.67 (2H, m), 1.99 (2H, t), 2.20-2.33 (2H, m), 4.24-4.42 (2H, m), 6.60 (1H, br d), 6.64 (1H, dd), 7.13 (1H, t), 7.45 (1H, dd), 7.56 (1H, d), 7.69 (1H, dd), 7.78 (1H, d), 7.97 (1H, d), 8.35 (1H, dd).

### Example 90

### 6′-(((1S,3S)-3-((5-(Difluoromethoxy)pyrazin-2-yl)amino)cyclopentyl)amino)-2-oxo-2H-[1,3′-bipyridine]-3-carbonitrile—compound 90

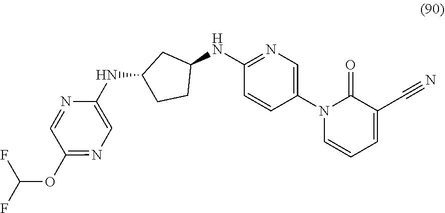 [SMILES: N#Cc1cccn(-c2ccc(N[C@H]3CC[C@H](Nc4cnc(OC(F)F)cn4)C3)nc2)c1=O]

Cs₂CO₃ (450 mg, 1.38 mmol) was added to 6′-(((1S,3S)-3-aminocyclopentyl)amino)-2-oxo-2H-[1,3′-bipyridine]-3-carbonitrile 4HCl compound i-35b (122 mg, 0.28 mmol), 2-chloro-5-(difluoromethoxy)pyrazine compound i-19a (100 mg, 0.55 mmol) and Pd-PEPPSI-IpentCl 2-methylpyridine (11.6 mg, 0.01 mmol) in DMF (5 mL) at 20° C. The resulting mixture was stirred at 100° C. for 15 h under a nitrogen atmosphere. The reaction mixture was diluted with water (75 mL) and extracted with EtOAc (5×50 mL). The organic layers were combined and washed with sat. brine (5×100 mL). The organic layer was dried over Na₂SO₄, filtered and evaporated. The residue was purified by preparative TLC (100% EtOAc) followed by preparative HPLC (PrepMethod B, gradient: 35-53%) to give the title compound (6 mg, 5%) as a pale-yellow solid. HRMS (ESI) m/z [M+H]⁺ calcd for C₂₁H₂₀F₂N₇O₂: 440.1640, found: 440.1662. ¹H NMR (300 MHz, DMSO-d₆) δ ppm 1.48-1.62 (2H, m), 1.92 (2H, t), 2.12-2.25 (2H, m), 4.15-4.48 (2H, m), 5.25-5.43 (1H, m), 6.49 (1H, t), 6.59-6.72 (1H, m), 7.13-7.27 (1H, m), 7.33 (1H, t), 7.51-7.58 (1H, m), 7.61 (1H, d), 7.92 (1H, d), 7.98-8.12 (2H, m), 8.24 (1H, dd).

### Example 91

### 6′-(((1S,3S)-3-((5-(Difluoromethoxy)-6-methylpyrazin-2-yl)amino)cyclopentyl)amino)-2H-[1,3′-bipyridin]-2-one—compound 91

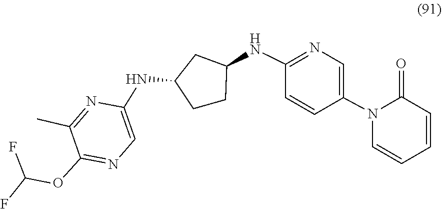 [SMILES: Cc1nc(N[C@H]2CC[C@H](Nc3ccc(-n4ccccc4=O)cn3)C2)cnc1OC(F)F]

5-Bromo-2-(difluoromethoxy)-3-methylpyrazine compound i-55a (120 mg, 0.50 mmol) was added to 6′-(((1S,3S)-3-aminocyclopentyl)amino)-2H-[1,3′-bipyridin]-2-one compound i-1c (90 mg, 0.33 mmol), Pd-PEPPSI-IpentCl 2-methylpyridine (14.1 mg, 0.02 mmol) and Cs₂CO₃ (436 mg, 1.34 mmol) in 1,4-dioxane (6 mL). The resulting mixture was stirred at 100° C. for 15 h under a nitrogen atmosphere. The reaction mixture was filtered through Celite. The filter cake was washed with EtOAc (3×20 mL) and the combined filtrates were concentrated under reduced pressure. The crude product was purified by preparative TLC (MeOH:DCM, 1:10) followed by preparative HPLC (PrepMethod E, gradient: 18-34%) to give the title compound (35 mg, 24%) as a white solid. HRMS (ESI) m/z [M+H]⁺ calcd for C₂₁H₂₃F₂N₆O₂: 429.1844, found: 429.1832. ¹H NMR (300 MHz, DMSO-d₆) δ ppm 1.40-1.58 (2H, m), 1.80-1.96 (2H, m), 2.06-2.24 (2H, m), 2.27 (3H, s), 4.15-4.40 (2H, m), 6.27 (1H, td), 6.44 (1H, d), 6.52 (1H, d), 6.96 (2H, t), 7.33 (1H, t), 7.36-7.42 (2H, m), 7.47 (1H, ddd), 7.60 (1H, dd), 7.92 (1H, d).

### Example 92

### 1-(6-(((1S,3S)-3-((5-(Difluoromethoxy)pyrazin-2-yl)amino)cyclopentyl)amino)pyridin-3-yl)-3-methylimidazolidine-2,4-dione—compound 92

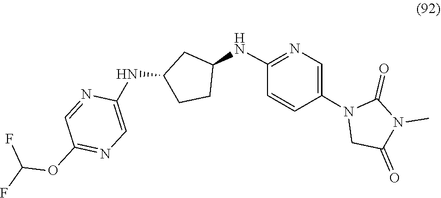 [SMILES: CN1C(=O)CN(c2ccc(N[C@H]3CC[C@H](Nc4cnc(OC(F)F)cn4)C3)nc2)C1=O]

1-(6-Chloropyridin-3-yl)-3-methylimidazolidine-2,4-dione compound i-23a (136 mg, 0.60 mmol) was added to (1S,3S)—N-(5-(difluoromethoxy)pyrazin-2-yl)cyclopentane-1,3-diamine 4TFA compound i-19c (464 mg, 0.66 mmol), Pd-PEPPSI-IpentCl 2-methylpyridine (25.4 mg, 0.03 mmol) and Cs₂CO₃ (1178 mg, 3.62 mmol) in 1,4-dioxane (10 mL) at 20° C. The resulting mixture was stirred at 100° C. for 15 h under a nitrogen atmosphere. The reaction mixture was diluted with water (150 mL) and washed sequentially with EtOAc (6×100 mL). The organic layer was dried over Na₂SO₄, filtered and evaporated. The residue was purified by preparative TLC (7 M NH₃ in MeOH:DCM, 1:20) followed by preparative HPLC (PrepMethod D, gradient: 28-48%) to give the title compound (46.6 mg, 17%) as a white solid. HRMS (ESI) m/z [M+H]⁺ calcd for C₁₉H₂₂F₂N₇O₃: 434.1746, found: 434.1766. ¹H NMR (300 MHz, DMSO-d₆) δ ppm 1.39-1.55 (2H, m), 1.85 (2H, t), 2.06-2.20 (2H, m), 2.91 (3H, s), 4.15-4.32 (2H, m), 4.37 (2H, s), 6.48 (1H, d), 6.62 (1H, d), 7.13 (1H, d), 7.32 (1H, t), 7.59 (1H, d), 7.62 (1H, dd), 7.89 (1H, d), 8.09 (1H, d).

### Example 93

### 3-(6-(((1S,3S)-3-((5-(Difluoromethoxy)pyrazin-2-yl)amino)cyclopentyl)amino)pyridin-3-yl)-1-methylimidazolidine-2,4-dione—compound 93

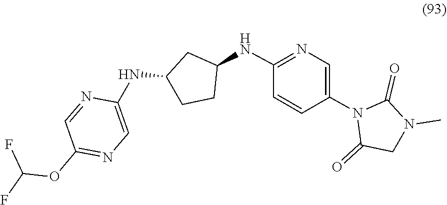 [SMILES: CN1CC(=O)N(c2ccc(N[C@H]3CC[C@H](Nc4cnc(OC(F)F)cn4)C3)nc2)C1=O]

Cs₂CO₃ (520 mg, 1.60 mmol) was added to (1S,3S)—N-(5-(difluoromethoxy)pyrazin-2-yl)cyclopentane-1,3-diamine 2.8TFA compound i-19c (300 mg, 0.53 mmol), 3-(6-chloropyridin-3-yl)-1-methylimidazolidine-2,4-dione compound i-7b (180 mg, 0.80 mmol) and Pd-PEPPSI-IpentCl 2-methylpyridine (22.4 mg, 0.03 mmol) in 1,4-dioxane (10 mL) at 25° C. The resulting solution was stirred at 100° C. for 16 h under a nitrogen atmosphere. The solvent was removed under reduced pressure. The residue was poured into water (50 mL) and extracted with EtOAc (2×100 mL). The organic layer was dried over Na₂SO₄, filtered and evaporated. The residue was purified by preparative TLC (100% EtOAc) followed by preparative HPLC (PrepMethod M, gradient: 35-55%) to give the title compound (40 mg, 17%) as a white solid. HRMS (ESI) m/z [M+H]⁺ calcd for C₁₉H₂₂F₂N₇O₃: 434.1746, found: 434.1736. ¹H NMR (300 MHz, DMSO-d₆) δ ppm 1.40-1.56 (2H, m), 1.87 (2H, t), 2.08-2.21 (2H, m), 2.90 (3H, s), 4.07 (2H, s), 4.15-4.37 (2H, m), 6.51 (1H, d), 6.90 (1H, d), 7.14 (1H, d), 7.26 (1H, dd), 7.32 (1H, t), 7.59 (1H, d), 7.84 (1H, d), 7.90 (1H, d).

### Example 94

### 6′-(((1S,3S)-3-((5-(Difluoromethoxy)pyrazin-2-yl)amino)cyclopentyl)amino)-3-methoxy-2H-[1,3′-bipyridin]-2-one—compound 94

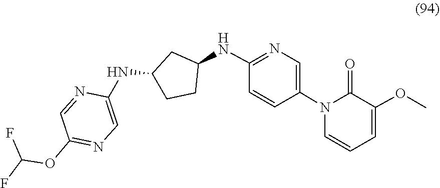 [SMILES: COc1cccn(-c2ccc(N[C@H]3CC[C@H](Nc4cnc(OC(F)F)cn4)C3)nc2)c1=O]

6′-Chloro-3-methoxy-2H-[1,3′-bipyridin]-2-one compound i-25a (150 mg, 0.63 mmol) was added to (1S,3S)—N-(5-(difluoromethoxy)pyrazin-2-yl)cyclopentane-1,3-diamine 8TFA compound i-19c (1466 mg, 1.27 mmol), Pd-PEPPSI-IpentCl 2-methylpyridine (26.7 mg, 0.03 mmol) and Cs₂CO₃ (1652 mg, 5.07 mmol) in 1,4-dioxane (10 mL) at 20° C. The resulting mixture was stirred at 100° C. for 15 h under a nitrogen atmosphere. The reaction mixture was diluted with EtOAc (80 mL) and washed sequentially with water (3×25 mL) and sat brine (3×15 mL). The organic layer was dried over Na₂SO₄, filtered and evaporated. The residue was purified by preparative TLC (7 M NH₃ in MeOH:DCM, 1:20) followed by preparative HPLC (PrepMethod B, gradient: 30-50%) to give the title compound (98 mg, 34%) as a white solid. HRMS (ESI) m/z [M+H]⁺ calcd for C₂₁H₂₃F₂N₆O₃: 445.1794, found: 445.1782. ¹H NMR (300 MHz, DMSO-d₆) δ ppm 1.41-1.59 (2H, m), 1.89 (2H, t), 2.08-2.24 (2H, m), 3.72 (3H, s), 4.16-4.40 (2H, m), 6.19 (1H, t), 6.52 (1H, d), 6.86 (1H, dd), 6.94 (1H, d), 7.10-7.19 (2H, m), 7.32 (1H, t), 7.38 (1H, dd), 7.60 (1H, d), 7.87-7.94 (2H, d).

### Example 95

### 6′-(((1S,3S)-3-((5-(Difluoromethoxy)pyrazin-2-yl)amino)cyclopentyl)amino)-5-methoxy-2H-[1,3′-bipyridin]-2-one—compound 95

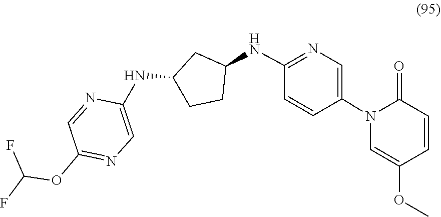 [SMILES: COc1ccc(=O)n(-c2ccc(N[C@H]3CC[C@H](Nc4cnc(OC(F)F)cn4)C3)nc2)c1]

6′-Chloro-5-methoxy-2H-[1,3′-bipyridin]-2-one compound i-26a (150 mg, 0.63 mmol) was added to (1S,3S)—N-(5-(difluoromethoxy)pyrazin-2-yl)cyclopentane-1,3-diamine 7TFA compound i-19c (1321 mg, 1.27 mmol), Pd-PEPPSI-IpentCl 2-methylpyridine (27 mg, 0.03 mmol) and Cs₂CO₃ (2.07 g, 6.34 mmol) in 1,4-dioxane (20 mL) at 20° C. The resulting mixture was stirred at 100° C. for 15 h under a nitrogen atmosphere. The reaction mixture was poured into water (150 mL) and extracted with EtOAc (4×150 mL). The organic layer was dried over Na₂SO₄, filtered and evaporated. The residue was purified by preparative TLC (7 M NH₃ in MeOH:DCM=1:20) and then by preparative HPLC (PrepMethod B, gradient: 30-50%) to give the title compound (55.6 mg, 19%) as a white solid. HRMS (ESI) m/z [M+H]⁺ calcd for C₂₁H₂₃F₂N₆O₃: 445.1794, found: 445.1826. ¹H NMR (300 MHz, DMSO-d₆) δ ppm 1.40-1.61 (2H, m), 1.89 (2H, t), 2.06-2.25 (2H, m), 3.64 (3H, s), 4.15-4.42 (2H, m), 6.42 (1H, d), 6.53 (1H, d), 6.91 (1H, d), 7.14 (1H, d), 7.18 (1H, d), 7.28-7.46 (2H, m), 7.32 (1H, t), 7.60 (1H, br s), 7.90 (1H, br s), 7.97 (1H, br d).

### Example 96

### 3-(6-(((1S,3S)-3-((5-(Difluoromethoxy)pyrazin-2-yl)amino)cyclopentyl)amino)pyridin-3-yl)-1-methyl-1,3-dihydro-2H-imidazo[4,5-b]pyridin-2-one—compound 96

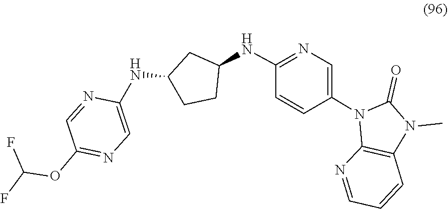 [SMILES: Cn1c(=O)n(-c2ccc(N[C@H]3CC[C@H](Nc4cnc(OC(F)F)cn4)C3)nc2)c2ncccc21]

Cs₂CO₃ (625 mg, 1.92 mmol) was added to 3-(6-chloropyridin-3-yl)-1-methyl-1,3-dihydro-2H-imidazo[4,5-b]pyridin-2-one compound i-24a (100 mg, 0.38 mmol), (1S,3S)—N¹-(5-(difluoromethoxy)pyrazin-2-yl)cyclopentane-1,3-diamine 2.8TFA compound i-19c (216 mg, 0.38 mmol) and Pd-PEPPSI-IpentCl 2-methylpyridine (16.1 mg, 0.02 mmol) in 1,4-dioxane (15 mL) at 20° C. The resulting mixture was stirred at 100° C. for 15 h under a nitrogen atmosphere. The reaction mixture was diluted with water (50 mL) and the aqueous layer was extracted with EtOAc (4×150 mL). The organic layer was dried over Na₂SO₄, filtered and evaporated. The residue was purified by preparative TLC (MeOH:DCM=1:20) followed by preparative HPLC (PrepMethod B, gradient: 34-54%) to give the title compound (55 mg, 30%) as a white solid. HRMS (ESI) m/z [M+H]⁺ calcd for C₂₂H₂₃F₂N₈O₂: 469.1906, found: 469.1920. ¹H NMR (300 MHz, DMSO-d₆) δ ppm 1.42-1.60 (2H, m), 1.90 (2H, t), 2.08-2.24 (2H, m), 3.40 (3H, s), 4.17-4.43 (2H, m), 6.58 (1H, d), 6.93 (1H, d), 7.10-7.19 (2H, m), 7.32 (1H, t), 7.50 (1H, dd), 7.57 (1H, dd), 7.60 (1H, d), 7.91 (1H, d), 7.93 (1H, dd), 8.09 (1H, d).

### Example 97

### 6′-(((1S,3S)-3-((5-(Difluoromethoxy)pyrazin-2-yl)amino)cyclopentyl)amino)-2′-methyl-2H-[1,3′-bipyridin]-2-one—compound 97

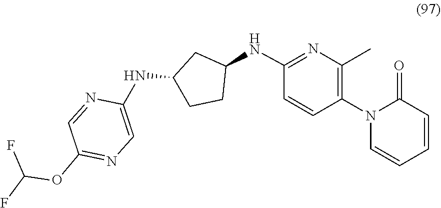 [SMILES: Cc1nc(N[C@H]2CC[C@H](Nc3cnc(OC(F)F)cn3)C2)ccc1-n1ccccc1=O]

According to general procedure GM2 the free base (1S,3S)—N-(5-(difluoromethoxy)pyrazin-2-yl)cyclopentane-1,3-diamine compound i-19c (63 mg, 0.26 mmol), 6′-chloro-2′-methyl-2H-[1,3′-bipyridin]-2-one compound i-102a (57 mg, 0.31 mmol), Cs₂CO₃ (252 mg, 0.77 mmol) and Pd PEPPSI-IpentCl 2-methylpyridine (17 mg, 0.02 mmol) were reacted in 1,4-dioxane (3 mL) at 100° C. for 2 h under a nitrogen atmosphere. Upon the alternative aqueous workup (EtOAc) followed by preparative TLC (EtOAc) and preparative HPLC (PrepMethod D, gradient: 25-55%) the title compound (18 mg, 16%) was obtained as a white solid. HRMS (ESI) m/z [M+H]⁺ calcd for C₂₁H₂₃F₂N₆O₂: 429.1844, found: 429.1872. ¹H NMR (300 MHz, DMSO-d₆) δ ppm 1.37-1.61 (2H, m), 1.85-1.95 (2H, m), 2.02 (3H, s), 2.08-2.29 (2H, m), 4.13-4.40 (2H, m), 6.27 (1H, dt), 6.31-6.49 (2H, m), 6.87 (1H, brs), 7.06-7.56 (5H, m), 7.59 (1H, d), 7.90 (1H, d). ¹⁹F NMR (282 MHz, DMSO-d₆) δ ppm −83.8.

### Example 98

### 3-(6-(((1S,3S)-3-((5-Chloropyrazin-2-yl)amino)cyclopentyl)amino)pyridin-3-yl)-1-methyl-1,3-dihydro-2H-imidazo[4,5-b]pyridin-2-one—compound 98

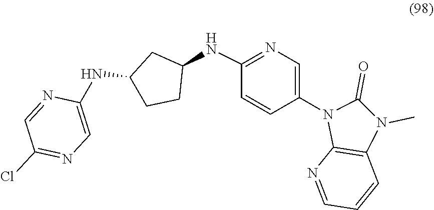 [SMILES: Cn1c(=O)n(-c2ccc(N[C@H]3CC[C@H](Nc4cnc(Cl)cn4)C3)nc2)c2ncccc21]

2,5-Dichloropyrazine (CAS Reg. No. 19745-07-4) (67.2 mg, 0.45 mmol) was added to 3-(6-(((1S,3S)-3-aminocyclopentyl)amino)pyridin-3-yl)-1-methyl-1,3-dihydro-2H-imidazo[4,5-b]pyridin-2-one×13HCl compound i-40b (360 mg, 0.45 mmol), Pd-PEPPSI-IpentCl 2-methylpyridine (19 mg, 0.02 mmol) and Cs₂CO₃ (1175 mg, 3.61 mmol) in 1,4-dioxane (20 mL) at 20° C. The resulting mixture was stirred at 100° C. for 15 h under a nitrogen atmosphere. The reaction mixture was diluted with water (50 mL) and the aqueous layer was extracted with EtOAc (4×150 mL). The organic layer was dried over Na₂SO₄, filtered and evaporated. The residue was purified by preparative TLC (7 M NH₃ in MeOH:DCM=1:20) followed by preparative HPLC (PrepMethod M, gradient: 30-60%) to give the title compound (65.3 mg, 33%) as a white solid. HRMS (ESI) m/z [M+H]⁺ calcd for C₂₁H₂₂ClN₈O: 437.1600, found: 437.1604. ¹H NMR (300 MHz, DMSO-d₆) δ ppm 1.42-1.60 (2H, m), 1.90 (2H, t), 2.09-2.24 (2H, m), 3.40 (3H, s), 4.18-4.41 (2H, m), 6.58 (1H, d), 6.94 (1H, d), 7.13 (1H, dd), 7.42 (1H, d), 7.50 (1H, dd), 7.57 (1H, dd), 7.72 (1H, d), 7.93 (1H, dd), 8.04 (1H, d), 8.09 (1H, d).

### Example 99

### 6′-(((1S,3S)-3-((5-Chloropyrazin-2-yl)amino)cyclopentyl)amino)-2-oxo-2H-[1,3′-bipyridine]-5-carbonitrile—compound 99

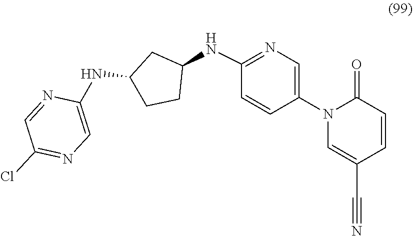 [SMILES: N#Cc1ccc(=O)n(-c2ccc(N[C@H]3CC[C@H](Nc4cnc(Cl)cn4)C3)nc2)c1]

6′-(((1S,3S)-3-Aminocyclopentyl)amino)-2-oxo-2H-[1,3′-bipyridine]-5-carbonitrile HCl compound i-33b (50.0 mg, 0.15 mmol) was added to Pd-PEPPSI-IpentCl 2-methylpyridine (6.3 mg, 7.5 μmol), 2,5-dichloropyrazine (CAS Reg. No. 19745-07-4) (44.9 mg, 0.30 mmol) and Cs₂CO₃ (147 mg, 0.45 mmol) in 1,4-dioxane (5 mL). The resulting mixture was stirred at 100° C. for 15 h under a nitrogen atmosphere. The reaction mixture was filtered through Celite. The filter cake was washed with EtOAc (3×20 mL) and the combined filtrates concentrated under reduced pressure. The crude product was purified by preparative TLC (MeOH:DCM=1:10) followed by preparative HPLC (PrepMethod J, gradient: 15-35%) to give the title compound (6 mg, 9%) as a white solid. HRMS (ESI) m/z [M+H]⁺ calcd for C₂₀H₁₉ClN₇O: 408.1334, found: 408.1340. ¹H NMR (300 MHz, CD₃OD) δ ppm 1.53-1.67 (2H, m), 1.99 (2H, t), 2.19-2.34 (2H, m), 4.26-4.41 (2H, m), 6.60 (1H, br d), 6.64 (1H, dd), 7.45 (1H, dd), 7.66 (1H, d), 7.69 (1H, dd), 7.95 (1H, d), 7.97 (1H, d), 8.35 (1H, dd).

### Example 100

### 3-(6-(((1S,3S)-3-((5-Chloropyrazin-2-yl)amino)cyclopentyl)amino)pyridin-3-yl)-1-methylimidazolidine-2,4-dione—compound 100

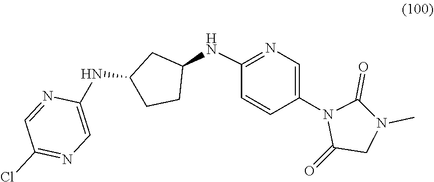 [SMILES: CN1CC(=O)N(c2ccc(N[C@H]3CC[C@H](Nc4cnc(Cl)cn4)C3)nc2)C1=O]

Cs₂CO₃ (1.02 g, 3.13 mmol) was added to 3-(6-(((1S,3S)-3-aminocyclopentyl)amino)pyridin-3-yl)-1-methylimidazolidine-2,4-dione 2.8HCl compound i-41b (244.7 mg, 0.63 mmol), 2,5-dichloropyrazine (186 mg, 1.25 mmol) and Pd-PEPPSI-IpentCl 2-methylpyridine (42 mg, 0.05 mmol) in 1,4-dioxane (4 mL) and DMA (2 mL) at 25° C. The resulting suspension was stirred at 100° C. for 16 h under a nitrogen atmosphere. The solvent was removed under reduced pressure, the residue poured into water (100 mL) and extracted with EtOAc (2×50 mL). The organic layers were combined and washed sequentially with sat. brine (3×50 mL), dried over Na₂SO₄, filtered and evaporated. The residue was purified by preparative TLC (7 M NH₃ in MeOH:DCM=1:20) followed by preparative HPLC (PrepMethod B, gradient: 25-45%) to give the title compound (20 mg, 8%) as a white solid. HRMS (ESI) m/z [M+H]⁺ calcd for C₁₈H₂₁ClN₇O₂: 402.1440, found: 402.1456. ¹H NMR (300 MHz, DMSO-d₆) δ ppm 1.40-1.57 (2H, m), 1.87 (2H, t), 2.08-2.21 (2H, m), 2.90 (3H, s), 4.07 (2H, s), 4.15-4.38 (2H, m), 6.50 (1H, d), 6.90 (1H, d), 7.26 (1H, dd), 7.40 (1H, d), 7.72 (1H, d), 7.84 (1H, d), 8.03 (1H, d).

### Example 102

### 6′-(((1S,3S)-3-((5-Chloro-6-methylpyrazin-2-yl)amino)cyclopentyl)amino)-2H-[1,3′-bipyridin]-2-one—compound 102

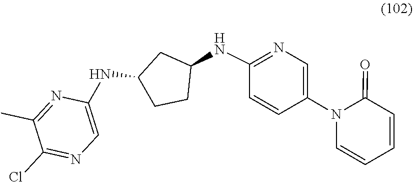 [SMILES: Cc1nc(N[C@H]2CC[C@H](Nc3ccc(-n4ccccc4=O)cn3)C2)cnc1Cl]

6′-(((1S,3S)-3-Aminocyclopentyl)amino)-2H-[1,3′-bipyridin]-2-one compound i-1c (140 mg, 0.52 mmol) was added to 5-bromo-2-chloro-3-methylpyrazine (CAS Reg. No. 112930-94-6) (269 mg, 1.29 mmol), Pd-PEPPSI-IpentCl 2-methylpyridine (21.78 mg, 0.03 mmol) and Cs₂CO₃ (844 mg, 2.59 mmol) in 1,4-dioxane (5 mL). The resulting mixture was stirred at 100° C. for 15 h under a nitrogen atmosphere. The reaction mixture was filtered through Celite. The filter cake was washed with EtOAc (3×20 mL). The filtrate was concentrated. The residue was purified by preparative TLC (MeOH:DCM, 1:15) followed by preparative HPLC (PrepMethod D, gradient: 5-30%) to give the title compound (47 mg, 23%) as a white solid. HRMS (ESI) m/z [M+H]⁺ calcd for C₂₀H₂₂ClN₆O: 397.1538, found: 397.1534. ¹H NMR (300 MHz, DMSO-d₆) δ ppm 1.41-1.59 (2H, m), 1.81-1.97 (2H, m), 2.08-2.24 (2H, m), 2.35 (3H, s), 4.18-4.40 (2H, m), 6.27 (1H, td), 6.44 (1H, ddd), 6.52 (1H, d), 6.93 (1H, d), 7.24 (1H, d), 7.40 (1H, dd), 7.47 (1H, ddd), 7.55 (1H, d), 7.59 (1H, ddd), 7.92 (1H, d).

### Example 104

### 6′-(((1S,3S)-3-((5-Cyclopropylpyrazin-2-yl)amino)cyclopentyl)amino)-2-oxo-2H-[1,3′-bipyridine]-3-carbonitrile—compound 104

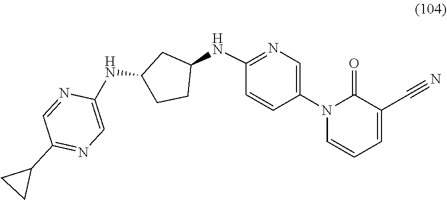 [SMILES: N#Cc1cccn(-c2ccc(N[C@H]3CC[C@H](Nc4cnc(C5CC5)cn4)C3)nc2)c1=O]

Cs₂CO₃ (513 mg, 1.58 mmol) was added to 6′-(((1S,3S)-3-aminocyclopentyl)amino)-2-oxo-2H-[1,3′-bipyridine]-3-carbonitrile×4HCl compound i-35b (139 mg, 0.32 mmol), 2-bromo-5-cyclopropylpyrazine (CAS Reg. No. 1086382-78-6) (69.0 mg, 0.35 mmol) and Pd-PEPPSI-IpentCl 2-methylpyridine (CAS Reg. No. 1612891-29-8) (13.2 mg, 0.02 mmol) in DMF (7 mL) at rt. The resulting mixture was stirred at 100° C. for 15 h under nitrogen, diluted with water (90 mL) and extracted with EtOAc (5×150 mL). The organic layers were combined and washed with brine (5×300 mL), dried (Na₂SO₄), filtered and evaporated to afford the crude product. The residue was first purified by preparative TLC (EtOAc) and then by preparative HPLC (PrepMethod D, gradient: 21-45%) to afford the title compound (50 mg, 38%) as a yellow solid. HRMS (ESI) m/z [M+H]⁺ calcd for C₂₃H₂₄N₇O: 414.2036, found: 414.2032. ¹H NMR (300 MHz, DMSO-d₆) δ ppm 0.72 (2H, dt), 0.78-0.88 (2H, m), 1.41-1.57 (2H, m), 1.81-1.98 (3H, m), 2.09-2.23 (2H, m), 4.18-4.41 (2H, m), 6.43-6.59 (2H, m), 6.84 (1H, d), 7.07 (1H, d), 7.46 (1H, dd), 7.78 (1H, d), 7.88 (1H, d), 7.98 (1H, d), 8.06 (1H, dd), 8.23 (1H, dd).

### Example 105

### 3-(6-(((1S,3S)-3-((5-Cyclopropylpyrazin-2-yl)amino)cyclopentyl)amino)pyridin-3-yl)-1-methyl-1,3-dihydro-2H-imidazo[4,5-b]pyridin-2-one—compound 105

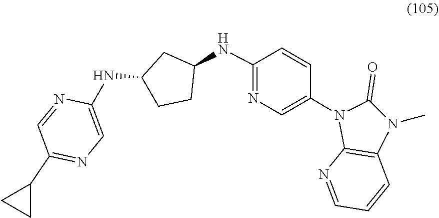 [SMILES: Cn1c(=O)n(-c2ccc(N[C@H]3CC[C@H](Nc4cnc(C5CC5)cn4)C3)nc2)c2ncccc21]

3-(6-(((1S,3S)-3-Aminocyclopentyl)amino)pyridin-3-yl)-1-methyl-1,3-dihydro-2H-imidazo[4,5-b]pyridin-2-one×3 TFA compound i-40b (200 mg, 0.30 mmol) was added to a mixture of 2-bromo-5-cyclopropylpyrazine (CAS Reg. No. 1086382-78-6) (60 mg, 0.30 mmol), Cs₂CO₃ (489 mg, 1.50 mmol) and Pd-PEPPSI-IpentCl 2-methylpyridine (CAS Reg. No. 1612891-29-8) (13 mg, 0.02 mmol) in 1,4-dioxane (10 mL) at rt and the resulting mixture was stirred at 100° C. for 15 h under nitrogen. The reaction mixture was poured into brine (150 mL), extracted with EtOAc (4×100 mL), the organic layers were combined and washed with saturated brine (3×100 mL), dried (Na₂SO₄), filtered and evaporated to afford the crude product. The residue was first purified by preparative TLC (MeOH:DCM=1:20) and then by preparative HPLC (PrepMethod D, gradient: 21-51%) to afford (66 mg, 50%) of the title compound as a white solid. HRMS (ESI) m/z [M+H]⁺ calcd for C₂₄H₂₇N₈O: 443.2302, found: 443.2322. ¹H NMR (300 MHz, DMSO-d₆) δ ppm 0.67-0.76 (2H, m), 0.77-0.85 (2H, m), 1.39-1.6 (2H, m), 1.84-1.99 (3H, m), 2.16 (2H, q), 3.41 (3H, s), 4.19-4.4 (2H, m), 6.59 (1H, d), 6.84 (1H, d), 6.93 (1H, d), 7.09-7.19 (1H, m), 7.45-7.62 (2H, m), 7.79 (1H, d), 7.86-7.98 (2H, m), 8.09 (1H, d).

### Example 106

### 6′-(((1S,3S)-3-((5-Cyclopropylpyrazin-2-yl)amino)cyclopentyl)amino)-3-methoxy-2H-[1,3′-bipyridin]-2-one—compound 106

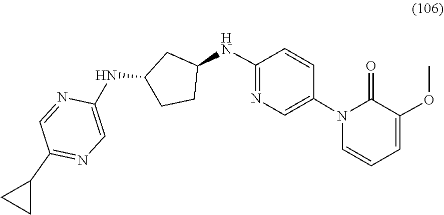 [SMILES: COc1cccn(-c2ccc(N[C@H]3CC[C@H](Nc4cnc(C5CC5)cn4)C3)nc2)c1=O]

6′-(((1S,3S)-3-aminocyclopentyl)amino)-3-methoxy-2H-[1,3′-bipyridin]-2-one×4TFA compound i-3b (200 mg, 0.26 mmol) was added to 2-bromo-5-cyclopropylpyrazine (CAS Reg. No. 1086382-78-6) (53 mg, 0.26 mmol), Cs₂CO₃ (431 mg, 1.32 mmol) and Pd-PEPPSI-IpentCl 2-methylpyridine (11 mg, 0.01 mmol) in 1,4-dioxane (10 mL) at rt and the resulting mixture was stirred at 100° C. for 15 h under nitrogen. The reaction mixture was poured into brine (150 mL), extracted with EtOAc (3×100 mL), the organic layers were combined and washed with saturated brine (3×100 mL), dried (Na₂SO₄), filtered and evaporated to afford the crude product. The residue was first purified by preparative TLC (MeOH:DCM=1:20) and then by preparative HPLC (PrepMethod D, gradient: 18-48%) to afford (45 mg, 41%) of the title compound as a white solid. HRMS (ESI) m/z [M+H]⁺ calcd for C₂₃H₂₇N₆O₂: 419.2190, found: 419.2194. ¹H NMR (400 MHz, DMSO-d₆) δ ppm 0.66-0.86 (4H, m), 1.41-1.57 (2H, m), 1.83-1.95 (3H, m), 2.01-2.20 (2H, m), 3.72 (3H, s), 4.20-4.36 (2H, m), 6.20 (1H, t), 6.53 (1H, d), 6.86 (2H, t), 6.94 (1H, d), 7.17 (1H, dd), 7.38 (1H, dd), 7.79 (1H, s), 7.86-7.93 (2H, m).

### Example 107

### 6′-(((1S,3S)-3-((5-Cyclopropylpyrazin-2-yl)amino)cyclopentyl)amino)-5-methoxy-2H-[1,3′-bipyridin]-2-one—compound 107

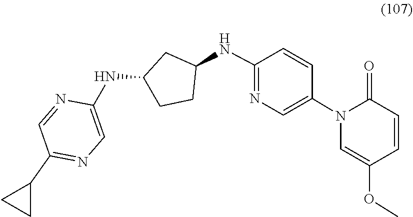 [SMILES: COc1ccc(=O)n(-c2ccc(N[C@H]3CC[C@H](Nc4cnc(C5CC5)cn4)C3)nc2)c1]

6′-(((1S,3S)-3-aminocyclopentyl)amino)-5-methoxy-2H-[1,3′-bipyridin]-2-one×2TFA compound i-42b (220 mg, 0.42 mmol) was added to 2-bromo-5-cyclopropylpyrazine (CAS Reg. No. 1086382-78-6) (124 mg, 0.62 mmol), Pd-PEPPSI-IpentCl 2-methylpyridine (CAS Reg. No. 1612891-29-8) (18 mg, 0.02 mmol) and Cs₂CO₃ (678 mg, 2.08 mmol) in 1,4-dioxane (15 mL) at rt and the resulting suspension was stirred at 100° C. for 18 h under nitrogen. The reaction mixture was concentrated and diluted with EtOAc (100 mL), washed sequentially with water (3×45 mL) and saturated brine (3×35 mL). The organic layer was dried (Na₂SO₄), filtered and evaporated and the residue was first purified by preparative TLC (7 M NH₃ in MeOH:DCM=1:15) followed by preparative HPLC (PrepMethod D, gradient 23-43%) to afford (65 mg, 37%) of the title compound as a white solid. HRMS (ESI) m/z [M+H]⁺ calcd for C₂₃H₂₇N₆O₂: 419.2190, found: 419.2190. ¹H NMR (300 MHz, DMSO-d₆) δ ppm 0.64-0.74 (2H, m), 0.74-0.87 (2H, m), 1.37-1.57 (2H, m), 1.8-1.98 (3H, m), 2.03-2.21 (2H, m), 3.62 (3H, s), 4.16-4.37 (2H, m), 6.41 (1H, d), 6.51 (1H, d), 6.82 (1H, d), 6.90 (1H, d), 7.17 (1H, d), 7.29-7.45 (2H, m), 7.77 (1H, d), 7.86 (1H, d), 7.95 (1H, d).

### Example 108

### 3-Chloro-6′-(((1S,3S)-3-((5-Cyclopropylpyrazin-2-yl)amino)cyclopentyl)amino)-2H-[1,3′-bipyridin]-2-one—compound 108

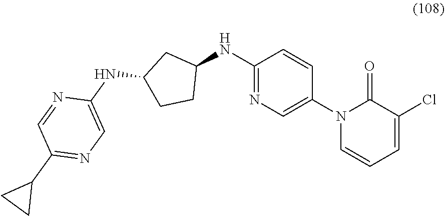 [SMILES: O=c1c(Cl)cccn1-c1ccc(N[C@H]2CC[C@H](Nc3cnc(C4CC4)cn3)C2)nc1]

2-Bromo-5-cyclopropylpyrazine (CAS Reg. No. 1086382-78-6) (82 mg, 0.41 mmol) was added to 6′-(((1S,3S)-3-aminocyclopentyl)amino)-3-chloro-2H-[1,3′-bipyridin]-2-one×2HCl compound i-43b (120 mg, 0.32 mmol), Pd-PEPPSI-IpentCl 2-methylpyridine (13 mg, 0.02 mmol) and Cs₂CO₃ (311 mg, 0.95 mmol) in 1,4-dioxane (20 mL) at rt under nitrogen and the resulting mixture was stirred at 100° C. for 18 h. The reaction mixture was concentrated and diluted with EtOAc (300 mL) and washed sequentially with brine (3×75 mL). The organic layer was dried (Na₂SO₄), filtered and evaporated to afford the crude product. The residue was first purified by preparative TLC (EtOAc:PE=1:5) and the by preparative HPLC (PrepMethod Z8, gradient 23-53%) to afford (15 mg, 11%) of the title compound as a white solid. HRMS (ESI) m/z [M+H]⁺ calcd for C₂₂H₂₄ClN₆O: 423.1694, found: 423.1676. ¹H NMR (300 MHz, DMSO-d₆) δ ppm 0.66-0.75 (2H, m), 0.76-0.88 (2H, m), 1.39-1.59 (2H, m), 1.82-2 (3H, m), 2.05-2.23 (2H, m), 4.19-4.4 (2H, m), 6.31 (1H, t), 6.53 (1H, d), 6.84 (1H, d), 7.01 (1H, d), 7.44 (1H, dd), 7.66 (1H, dd), 7.79 (1H, d), 7.82 (1H, dd), 7.89 (1H, d), 7.96 (1H, d).

### Example 109

### 5-Chloro-6′-(((1S,3S)-3-((5-Cyclopropylpyrazin-2-yl)amino)cyclopentyl)amino)-2H-[1,3′-bipyridin]-2-one—compound 109

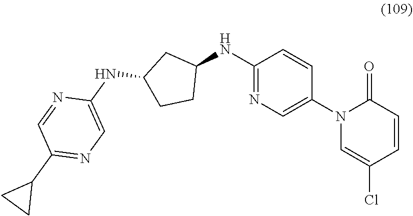 [SMILES: O=c1ccc(Cl)cn1-c1ccc(N[C@H]2CC[C@H](Nc3cnc(C4CC4)cn3)C2)nc1]

6′-(((1S,3S)-3-Aminocyclopentyl)amino)-5-chloro-2H-[1,3′-bipyridin]-2-one×2TFA compound i-44b (300 mg, 0.56 mmol) was added to 2-bromo-5-cyclopropylpyrazine (168 mg, 0.84 mmol), Pd-PEPPSI-IpentCl 2-methylpyridine (24 mg, 0.03 mmol) and Cs₂CO₃ (917 mg, 2.82 mmol) in 1,4-dioxane (10 mL) at rt and the resulting suspension was stirred at 100° C. for 18 h under nitrogen. The reaction mixture was concentrated and diluted with EtOAc (125 mL) and washed sequentially with water (3×50 mL) and brine (3×45 mL). The organic layer was dried (Na₂SO₄), filtered and evaporated to afford the crude product. The residue was first purified by preparative TLC (7 M NH₃ in MeOH:DCM=1:15) and then by preparative HPLC (PrepMethod D, gradient 25-55%) to afford (17 mg, 7%) of the title compound as a white solid. HRMS (ESI) m/z [M+H]⁺ calcd for C₂₂H₂₄ClN₆O: 423.1694, found: 423.1666. ¹H NMR (300 MHz, DMSO-d₆) δ ppm 0.68-0.75 (2H, m), 0.75-0.93 (2H, m), 1.43-1.55 (2H, m), 1.80-2.05 (3H, m), 2.14 (2H, brs), 4.20-4.43 (2H, m), 6.45-6.55 (2H, m), 6.84 (1H, d), 6.98 (1H, d), 7.41 (1H, dd), 7.55 (1H, dd), 7.78 (1H, d), 7.88 (1H, d), 7.91 (1H, d), 7.95 (1H, d).

### Example 110

### 6′-(((1S,3S)-3-((5-Cyclopropylpyrazin-2-yl)amino)cyclopentyl)amino)-2-oxo-2H-[1,3′-bipyridine]-5-carbonitrile—compound 110

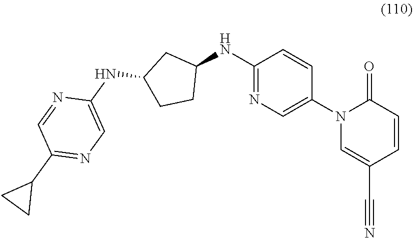 [SMILES: N#Cc1ccc(=O)n(-c2ccc(N[C@H]3CC[C@H](Nc4cnc(C5CC5)cn4)C3)nc2)c1]

(1S,3S)—N¹-(5-Cyclopropylpyrazin-2-yl)cyclopentane-1,3-diamine×3TFA compound i-45b (169 mg, 0.30 mmol) was added to 6′-chloro-2-oxo-2H-[1,3′-bipyridine]-5-carbonitrile compound i-8d (70 mg, 0.30 mmol), Cs₂CO₃ (394 mg, 1.21 mmol) and Pd-PEPPSI-IpentCl 2-methylpyridine (13 mg, 0.02 mmol) in 1,4-dioxane (10 mL) and the resulting mixture was stirred at 100° C. for 15 h under nitrogen. The reaction mixture was filtered through celite. The filter cake was washed with EtOAc (3×20 mL). The filtrate was concentrated under reduced pressure to afford the crude product. The crude product was first purified by preparative TLC (MeOH:DCM=1:10), and further purified by preparative HPLC (PrepMethod J, gradient 15-35%) to afford (72 mg, 58%) as a white solid. HRMS (ESI) m/z [M+H]⁺ calcd for C₂₃H₂₄N₇O: 414.2036, found: 414.1998. ¹H NMR (300 MHz, Methanol-d₄) δ ppm 0.60-1.00 (4H, m), 1.44-1.73 (2H, m), 1.88-2.06 (3H, m), 2.06-2.32 (2H, m), 4.35-4.50 (2H, m), 6.62 (2H, dd), 7.45 (1H, dd), 7.69 (1H, dd), 7.79 (1H, d), 7.97 (1H, d), 8.10 (1H, dd) 8.35 (1H, d).

### Example 111

### 6′-(((1S,3S)-3-((5-(Trifluoromethoxy)pyrazin-2-yl)amino)cyclopentyl)amino)-2H-[1,3′-bipyridin]-2-one—compound 111

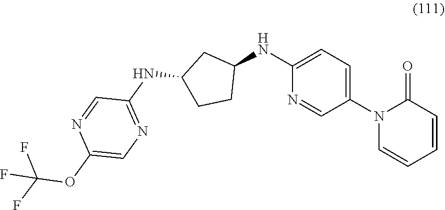 [SMILES: O=c1ccccn1-c1ccc(N[C@H]2CC[C@H](Nc3cnc(OC(F)(F)F)cn3)C2)nc1]

(1S,3S)—N¹-(5-(trifluoromethoxy)pyrazin-2-yl)cyclopentane-1,3-diamine compound i-46b (30 mg, 0.11 mmol), 6′-fluoro-2H-[1,3′-bipyridin]-2-one (CAS Reg. No. 2062643-92-7) compound i-100a (28 mg, 0.15 mmol) and Na₂CO₃ (49 mg, 0.46 mmol) in DMSO (2.5 mL) was stirred at 120° C. for 20 h. The mixture was cooled to rt and purified by preparative HPLC (PrepMethod H, gradient 20-80%) to afford (20 mg, 40%) of the title compound. HRMS (ESI) m/z [M+H]⁺ calcd for C₂₀H₂₀F₃N₆O₂: 433.1594, found: 433.1568. ¹H NMR (500 MHz, CDCl₃) 1.49-1.63 (2H, m), 2.01-2.05 (2H, m), 2.32 (2H, ddq), 4.27 (2H, dq), 4.89 (1H, d), 5.07 (1H, d), 6.24 (1H, td), 6.43 (1H, d), 6.64 (1H, d), 7.29 (1H, dd), 7.40 (1H, ddd), 7.49 (1H, dd), 7.56 (1H, d), 7.9-7.96 (1H, m), 8.03 (1H, d).

### Example 139

### 6′-(((1S,3S)-3-((7-Chloroquinoxalin-2-yl)amino)cyclopentyl)amino)-2H-[1,3′-bipyridin]-2-one—compound 139

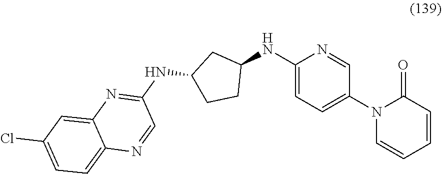 [SMILES: O=c1ccccn1-c1ccc(N[C@H]2CC[C@H](Nc3cnc4ccc(Cl)cc4n3)C2)nc1]

2,7-Dichloroquinoxaline (CAS Reg. No. 59489-31-5) (10 mg, 0.05 mmol) was added to a vial and transferred into the glovebox. NaOtBu (0.024 g, 0.25 mmol) was added followed by 6′-(((1S,3S)-3-aminocyclopentyl)amino)-2H-[1,3′-bipyridin]-2-one 3HCl compound i-1c (19 mg, 0.05 mmol), tBuXPhos Pd G3 (CAS Reg. No. 1447963-75-8) (1.99 mg, 2.50 μmol) and tBuXPhos (CAS Reg. No. 564483-19-8) (0.64 mg, 1.5 μmol) as a stock solutions in DMA (250 μL). The paradox plate was sealed and the reaction was carried out at 65° C. for 16 h. The reaction was cooled to rt. SiliaMetS Thiol (SH) Metal Scavenger (R51030B) (10 mg) was added and the mixture was stirred for at rt for 5 h. The reaction was diluted with DMSO, filtered and purified by preparative HPLC (PrepMethod Parallel C) to give the title compound (2 mg, 9%). HRMS (ESI) m/z [M+H]⁺ calcd for C₂₃H₂₂ClN₆O: 433.1538, found: 433.1540. ¹H NMR (600 MHz, DMSO-d₆) δ ppm 8.29 (s, 1H), 7.96 (d, 1H), 7.94 (dd, 1H), 7.75 (d, 1H), 7.60 (ddd, 1H), 7.54 (d, 1H), 7.48 (ddd, 1H), 7.41 (dd, 1H), 7.31 (dd, 1H), 6.98 (d, 1H), 6.55 (dd, 1H), 6.45 (ddd, 1H), 6.28 (td, 1H), 4.45-4.54 (m, 1H), 4.36-4.43 (m, 1H), 2.16-2.3 (m, 2H), 1.92-2.06 (m, 2H), 1.51-1.62 (m, 2H).

### Example 140

### 6′-(((1S,3S)-3-((5-Methoxypyrazin-2-yl)amino)cyclopentyl)amino)-2H-[1,3′-bipyridin]-2-one compound 140

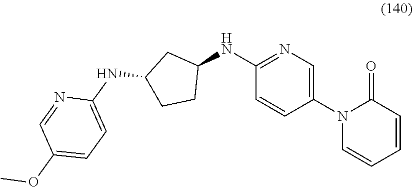 [SMILES: COc1ccc(N[C@H]2CC[C@H](Nc3ccc(-n4ccccc4=O)cn3)C2)nc1]

NaOtBu (381 mg, 3.97 mmol) was added to 2-bromo-5-methoxypyrazine (CAS Reg. No. 143250-10-6) (150 mg, 0.79 mmol), 6′-(((1S,3S)-3-aminocyclopentyl)amino)-2H-[1,3′-bipyridin]-2-one HCl compound i-1c (243 mg, 0.79 mmol), Pd₂(dba)₃ (72.7 mg, 0.08 mmol) and DavePhos (CAS Reg. No. 213697-53-1) (62.5 mg, 0.16 mmol) in 1,4-dioxane (5 mL) at 15° C. The resulting suspension was stirred at 100° C. for 16 h under a nitrogen atmosphere. The solvent was removed under reduced pressure, the residue poured into water (100 mL) and extracted with EtOAc (2×50 mL). The organic layers were combined and washed with sat. brine (3×50 mL), dried over Na₂SO₄, filtered and evaporated. The residue was purified by preparative TLC (EtOAc:PE=1:3) followed by preparative HPLC (PrepMethod D, gradient: 18-35%) to give the title compound (90 mg, 30%) as a white solid. HRMS (ESI) m/z [M+H]⁺ calcd for C₂₀H₂₃N₆O₂: 379.1876, found: 379.1882. ¹H NMR (300 MHz, DMSO-d₆) δ ppm 1.40-1.55 (2H, m), 1.86 (2H, t), 2.06-2.22 (2H, m), 3.76 (3H, s), 4.10-4.23 (1H, m), 4.25-4.39 (1H, m), 6.27 (1H, td), 6.44 (1H, br d), 6.50 (1H, d), 6.52 (1H, d), 6.93 (1H, d), 7.39 (1H, dd), 7.47 (1H, ddd), 7.52 (1H, d), 7.60 (1H, dd), 7.73 (1H, d), 7.91 (1H, d).

### Example 141

### 6′-(((1S,3S)-3-((6-Methylpyrazin-2-yl)amino)cyclopentyl)amino)-2H-[1,3′-bipyridin]-2-one—compound 141

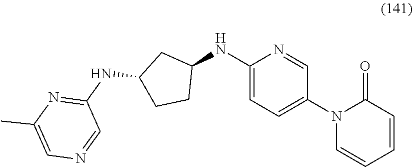 [SMILES: Cc1cncc(N[C@H]2CC[C@H](Nc3ccc(-n4ccccc4=O)cn3)C2)n1]

Pd₂(dba)₃-CHCl₃ (CAS Reg. No. 52522-40-4) (191 mg, 0.18 mmol) and XantPhos (CAS Reg. No. 161265-03-8) (107 mg, 0.18 mmol) were added to 6′-(((1S,3S)-3-aminocyclopentyl)amino)-2H-[1,3′-bipyridin]-2-one compound i-1c (250 mg, 0.92 mmol), 2-chloro-6-methylpyrazine (CAS Reg. No. 38557-71-0) (119 mg, 0.92 mmol) and Cs₂CO₃ (904 mg, 2.77 mmol) in toluene (5 mL) at 20° C. and the resulting suspension was stirred at 90° C. for 18 h under a nitrogen atmosphere. The reaction mixture was diluted with EtOAc (20 mL) and washed sequentially with water (3×50 mL). The organic layer was dried over Na₂SO₄, filtered and evaporated and the crude product purified by preparative HPLC (PrepMethod B, gradient: 11-38%) to give the title compound (19 mg, 5%) as a white solid. HRMS (ESI) m/z [M+H]⁺ calcd for C₂₀H₂₃N60:363.1928, found: 363.1916. ¹H NMR (300 MHz, DMSO-d₆) δ ppm 1.42-1.56 (2H, m), 1.89 (2H, t), 2.07-2.22 (2H, m), 2.24 (3H, s), 4.25-4.38 (2H, m), 6.27 (1H, t), 6.44 (1H, d), 6.52 (1H, d), 6.93 (1H, d), 6.99 (1H, d), 7.39 (1H, dd), 7.47 (1H, ddd), 7.52 (1H, s), 7.60 (1H, dd), 7.70 (1H, s), 7.92 (1H, d).

### Example 142

### 6-(((1S,3S)-3-((2-Oxo-2H-[1,3′-bipyridin]-6′-yl)amino)cyclopentyl)amino)pyrazine-2-carbonitrile—compound 142

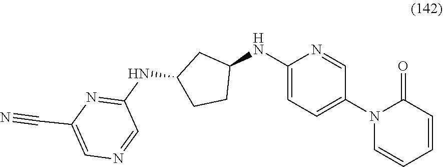 [SMILES: N#Cc1cncc(N[C@H]2CC[C@H](Nc3ccc(-n4ccccc4=O)cn3)C2)n1]

6-Chloropyrazine-2-carbonitrile (CAS Reg. No. 6863-74-7) (93 mg, 0.67 mmol) was added to 6′-(((1S,3S)-3-aminocyclopentyl)amino)-2H-[1,3′-bipyridin]-2-one compound i-1c (150 mg, 0.55 mmol) and DIPEA (291 μL, 1.66 mmol) in DMSO (5 mL) and the resulting mixture was stirred at 110° C. for 18 h. The reaction mixture was diluted with EtOAc (20 mL) and washed sequentially with water (3×50 mL). The organic layer was dried over Na₂SO₄, filtered and evaporated and the residue purified by preparative TLC (MeOH:DCM=1:20) followed by preparative HPLC (PrepMethod C, gradient: 2-47%) to give the title compound (38 mg, 18%) as a yellow solid. HRMS (ESI) m/z [M+H]⁺ calcd for C₂₀H₂₀N₇O: 374.1724, found: 374.1724. ¹H NMR (400 MHz, DMSO-d₆) δ ppm 1.48-1.58 (2H, m), 1.92 (2H, t), 2.14-2.22 (2H, m), 4.21-4.44 (2H, m), 6.28 (1H, td), 6.45 (1H, dd), 6.54 (1H, d), 6.99 (1H, d), 7.41 (1H, dd), 7.48 (1H, ddd), 7.59-7.63 (1H, m), 7.92-7.96 (2H, m), 8.16 (2H, d).

### Example 143

### 6′-(((1S,3S)-3-(Pyrido[2,3-b]pyrazin-3-ylamino)cyclopentyl)amino)-2H-[1,3′-bipyridin]-2-one—compound 143

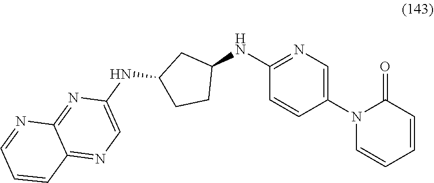 [SMILES: O=c1ccccn1-c1ccc(N[C@H]2CC[C@H](Nc3cnc4cccnc4n3)C2)nc1]

Na₂CO₃ (512 mg, 4.83 mmol) was added to 3-chloropyrido[2,3-b]pyrazine (CAS Reg. No. 155535-23-2) (200 mg, 1.21 mmol) and 6′-(((1S,3S)-3-aminocyclopentyl)amino)-2H-[1,3′-bipyridin]-2-one compound i-1c (653 mg, 2.42 mmol) in DMSO (5 mL) at 20° C. and the resulting suspension was stirred at 110° C. for 16 h under a nitrogen atmosphere. The solvent was removed under reduced pressure and the crude product purified by preparative HPLC (PrepMethod M, gradient: 15-35%) to give the title compound (0.3 g, 62%) as a white solid. HRMS (ESI) m/z [M+H]⁺ calcd for C₂₂H₂₂N₇O: 400.1880, found: 400.1876. ¹H NMR (300 MHz, DMSO-d₆) δ ppm 1.52-1.68 (2H, m), 2.01 (2H, t), 2.16-2.38 (2H, m), 4.33-4.62 (2H, m), 6.24-6.30 (1H, m), 6.40-6.47 (1H, m), 6.56 (1H, d), 7.01 (1H, d), 7.33 (1H, dd), 7.42 (1H, dd), 7.44-7.51 (1H, m), 7.58-7.66 (1H, m), 7.94 (1H, d), 8.11-8.16 (2H, m), 8.35 (1H, s), 8.70 (1H, dd).

### Example 144

### 6′-(((1S,3S)-3-((8-Methoxyquinoxalin-2-yl)amino)cyclopentyl)amino)-2H-[1,3′-bipyridin]-2-one—compound 144

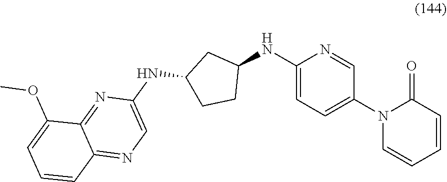 [SMILES: COc1cccc2ncc(N[C@H]3CC[C@H](Nc4ccc(-n5ccccc5=O)cn4)C3)nc12]

2-Chloro-8-methoxyquinoxaline (CAS Reg. No. 659729-70-1) (173 mg, 0.89 mmol) was added to 6′-(((1S,3S)-3-aminocyclopentyl)amino)-2H-[1,3′-bipyridin]-2-one compound i-1c (200 mg, 0.74 mmol) and Na₂CO₃ (314 mg, 2.96 mmol) in DMSO (5 mL) and the resulting mixture was stirred at 100° C. for 18 h. The reaction mixture was diluted with EtOAc (20 mL) and washed sequentially with water (5×50 mL). The organic layer was dried over Na₂SO₄, filtered and evaporated and the crude product purified by preparative HPLC (PrepMethod B, gradient: 20-45%) to give the title compound (65 mg, 20%) as a white solid. HRMS (ESI) m/z [M+H]⁺ calcd for C₂₄H₂₅N₆O₂: 429.2034, found: 429.2024. ¹H NMR (300 MHz, DMSO-d₆) δ ppm 1.40-1.64 (2H, m), 1.94-2.03 (2H, m), 2.13-2.32 (2H, m), 3.89 (3H, s), 4.34-4.43 (1H, m), 4.47-4.57 (1H, m), 6.27 (1H, td), 6.44 (1H, br d), 6.55 (1H, d), 6.99 (1H, d), 7.05 (1H, dd), 7.21 (1H, t), 7.34 (1H, dd), 7.41 (1H, dd), 7.47 (1H, ddd), 7.60 (1H, dd), 7.65 (1H, d), 7.93 (1H, d), 8.27 (1H, s).

### Example 145

### 6′-(((1S,3S)-3-((5-Fluoroquinoxalin-2-yl)amino)cyclopentyl)amino)-2H-[1,3′-bipyridin]-2-one—compound 145

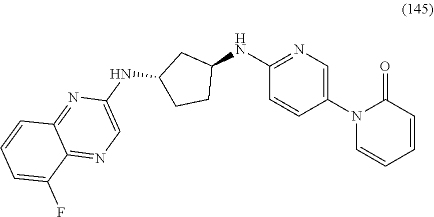 [SMILES: O=c1ccccn1-c1ccc(N[C@H]2CC[C@H](Nc3cnc4c(F)cccc4n3)C2)nc1]

2-Chloro-5-fluoroquinoxaline (CAS Reg. No. 55687-09-7) (81 mg, 0.44 mmol) was added to 6′-(((1S,3S)-3-aminocyclopentyl)amino)-2H-[1,3′-bipyridin]-2-one compound i-1c (100 mg, 0.37 mmol) and Na₂CO₃ (157 mg, 1.48 mmol) in DMSO (5 mL) and the resulting mixture was stirred at 100° C. for 18 h. The reaction mixture was diluted with EtOAc (20 mL) and washed sequentially with water (3×50 mL). The organic layer was dried over Na₂SO₄, filtered and evaporated and the crude product purified by preparative HPLC (PrepMethod D, gradient: 18-33%) to give the title compound (53 mg, 34%) as a white solid. HRMS (ESI) m/z [M+H]⁺ calcd for C₂₃H₂₂FN₆O: 417.1834, found: 417.1840. ¹H NMR (300 MHz, DMSO-d₆) δ ppm 1.49-1.65 (2H, m), 1.90-2.06 (2H, m), 2.14-2.31 (2H, m), 4.32-4.57 (2H, m), 6.27 (1H, td), 6.44 (1H, ddd), 6.54 (1H, dd), 6.99 (1H, d), 7.10 (1H, ddd), 7.35 (1H, dd), 7.41 (1H, dd), 7.44-7.54 (2H, m), 7.60 (1H, ddd), 7.93 (1H, d), 7.97 (1H, d), 8.30 (1H, s).

### Example 146

### 6′-(((1S,3S)-3-((6-Aminopyrido[2,3-b]pyrazin-3-yl)amino)cyclopentyl)amino)-2H-[1,3′-bipyridin]-2-one—compound 146

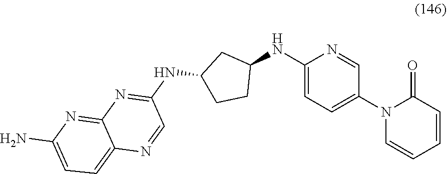 [SMILES: Nc1ccc2ncc(N[C@H]3CC[C@H](Nc4ccc(-n5ccccc5=O)cn4)C3)nc2n1]

3-Chloropyrido[2,3-b]pyrazin-6-amine (CAS Reg. No. 936760-62-2) (134 mg, 0.74 mmol) was added to 6′-(((1S,3S)-3-aminocyclopentyl)amino)-2H-[1,3′-bipyridin]-2-one compound i-1c (200 mg, 0.74 mmol) and Na₂CO₃ (235 mg, 2.22 mmol) in DMSO (1 mL) and the resulting mixture was stirred at 100° C. for 18 h. The reaction mixture was diluted with EtOAc (25 mL) and washed sequentially with water (3×50 mL). The organic layer was dried over Na₂SO₄, filtered and evaporated and the crude product purified by preparative HPLC (PrepMethod D, gradient: 14-29%) to give the title compound (7 mg, 2%) as a yellow solid. HRMS (ESI) m/z [M+H]⁺ calcd for C₂₂H₂₃N₈O: 415.1990, found: 415.1986. ¹H NMR (400 MHz, DMSO-d₆) δ ppm 1.47-1.60 (2H, m), 1.86-2.02 (2H, m), 2.12-2.26 (2H, m), 4.30-4.48 (2H, m), 6.27 (1H, td), 6.44 (1H, d), 6.47-6.57 (4H, m), 6.99 (1H, d), 7.40 (1H, dd), 7.47 (1H, ddd), 7.56 (1H, d), 7.61 (1H, dd), 7.65 (1H, d), 7.86 (1H, s), 7.92 (1H, d).

### Example 147

### 6′-(((1S,3S)-3-((5-Methylpyrazin-2-yl)amino)cyclopentyl)amino)-3-(trifluoromethyl)-2H-[1,3′-bipyridin]-2-one—compound 147

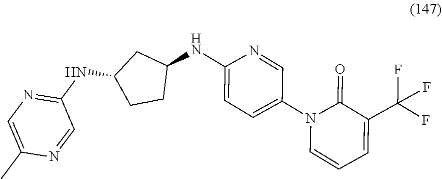 [SMILES: Cc1cnc(N[C@H]2CC[C@H](Nc3ccc(-n4cccc(C(F)(F)F)c4=O)cn3)C2)cn1]

3-(Trifluoromethyl)pyridin-2 (1H-one (CAS Reg. No. 22245-83-6) (124 mg, 0.76 mmol), Cu(I)I (96 mg, 0.51 mmol), (1S,3S)—N¹-(5-iodopyridin-2-yl)-NM-(5-methylpyrazin-2-yl)cyclopentane-1,3-diamine compound i-32a (100 mg, 0.25 mmol) and Cs₂CO₃ (330 mg, 1.01 mmol) were added to a vial. The vial was evacuated and filled with nitrogen (2×). A solution of rel-(1R,2R)—N¹,N²-dimethylcyclohexane-1,2-diamine (72 mg, 0.51 mmol) in toluene (2 mL) was added and the vial was again evacuated and filled with nitrogen (2×). The mixture was heated to 100° C. for 20 h, then cooled to rt, diluted with EtOAc and filtered through Celite. The filtrate was washed with water and brine, dried over Na₂SO₄, filtered and evaporated. The residue was purified by flash chromatography (gradient: 50-100% EtOAc:MeOH=95:5 in heptane, then isocratic: EtOAc:MeOH=95:5) followed by preparative HPLC (PrepMethod Parallel D) to give the title compound (20.4 mg, 18%). HRMS (ESI) m/z [M+H]⁺ calcd for C₂₁H₂₂F₃N₆O: 431.1802, found: 431.1798. ¹H NMR (600 MHz, DMSO-d₆) δ ppm 1.41-1.55 (2H, m), 1.84-1.9 (2H, m), 2.13-2.18 (2H, m), 2.23 (3H, s), 4.18-4.27 (1H, m), 4.27-4.37 (1H, m), 6.42 (1H, t), 6.54 (1H, dd), 6.74-6.81 (1H, m), 6.93 (1H, d), 7.41 (1H, dd), 7.73-7.83 (2H, m), 7.89 (1H, dd), 7.94 (1H, d), 7.97 (1H, dd).

### Example 148

### 6′-(((1S,3S)-3-((5-Methylpyrazin-2-yl)amino)cyclopentyl)amino)-3-(trifluoromethoxy)-2H-[1,3′-bipyridin]-2-one—compound 148

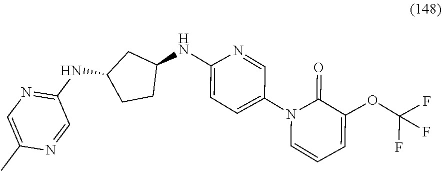 [SMILES: Cc1cnc(N[C@H]2CC[C@H](Nc3ccc(-n4cccc(OC(F)(F)F)c4=O)cn3)C2)cn1]

3-(Trifluoromethoxy)pyridin-2 (1H)-one compound i-56a (47 mg, 0.26 mmol), Cu(I)I (42.9 mg, 0.23 mmol), (1S,3S)—N¹-(5-iodopyridin-2-yl)-N³-(5-methylpyrazin-2-yl)cyclopentane-1,3-diamine compound i-32a (89 mg, 0.23 mmol) and Cs₂CO₃ (293 mg, 0.90 mmol) were added to a vial. The vial was evacuated and filled with nitrogen (2×). A solution of rel-(1R,2R)—N¹,N²-dimethylcyclohexane-1,2-diamine (32.0 mg, 0.23 mmol) in toluene (1.5 mL) was added and the vial was again evacuated and filled with nitrogen (2×). The mixture was heated to 110° C. for 5 h, then cooled to rt, diluted with EtOAc and filtered through Celite. The filtrate was washed with water and brine, dried over Na₂SO₄, filtered and evaporated. The residue was purified by preparative HPLC (PrepMethod SFC-D) to give the title compound (29 mg, 29%). HRMS (ESI) m/z [M+H]⁺ calcd for C₂₁H₂₂F₃N₆O₂: 447.1750, found: 447.1744. ¹H NMR (600 MHz, DMSO-d₆) b ppm 1.42-1.56 (2H, m), 1.88 (2H, t), 2.1-2.19 (2H, m), 2.23 (3H, s), 4.21-4.29 (1H, m), 4.29-4.38 (1H, m), 6.29-6.38 (1H, m), 6.51-6.57 (1H, m), 6.85 (1H, d), 6.96-7.05 (1H, m), 7.44 (1H, dd), 7.66-7.74 (2H, m), 7.77-7.86 (2H, m), 7.97 (1H, d).

### Example 149

### 5-(((1S,3S)-3-((3-Chloro-2-oxo-2H-[1,3′-bipyridin]-6′-yl)amino)cyclopentyl)amino)-N-methylpyrazine-2-carboxamide—compound 149

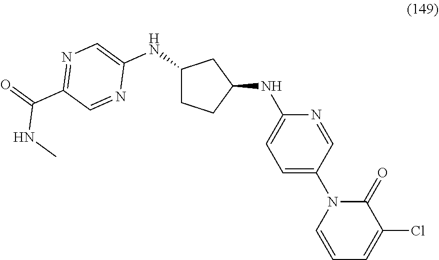 [SMILES: CNC(=O)c1cnc(N[C@H]2CC[C@H](Nc3ccc(-n4cccc(Cl)c4=O)cn3)C2)cn1]

Na₂CO₃ (293 mg, 2.76 mmol) was added to the HCl salt of 5-(((1S,3S)-3-aminocyclopentyl)amino)-N-methylpyrazine-2-carboxamide compound i-88c (150 mg, 0.55 mmol) and 3-chloro-6′-fluoro-2H-[1,3′-bipyridin]-2-one compound i-13a (149 mg, 0.66 mmol) in DMSO (15 mL) at rt and the resulting mixture was stirred at 120° C. for 15 h. The reaction mixture was concentrated, diluted with EtOAc (250 mL) and washed sequentially with brine (3×75 mL). The organic layer was dried (Na₂SO₄), filtered and evaporated to afford the crude product which was purified by preparative TLC (EtOAc) to afford (46 mg, 19%) of the title compound as a white solid. HRMS (ESI) m/z [M+H]⁺ calcd for C₂₁H₂₃ClN₇O₂: 440.1596, found: 440.1586. ¹H NMR (300 MHz, DMSO-d₆) δ 1.46-1.59 (2H, m), 1.92 (2H, t), 2.1-2.25 (2H, m), 2.76 (3H, d), 4.28-4.5 (2H, m), 6.31 (1H, t), 6.54 (1H, d), 7.05 (1H, d), 7.44 (1H, dd), 7.67 (1H, dd), 7.76-7.88 (3H, m), 7.97 (1H, d), 8.26 (1H, q), 8.51 (1H, d).

### Example 150

### 5-(((1S,3S)-3-((3-Methoxy-2-oxo-2H-[1,3′-bipyridin]-6′-yl)amino)cyclopentyl)amino)-N-methylpyrazine-2-carboxamide—compound 150

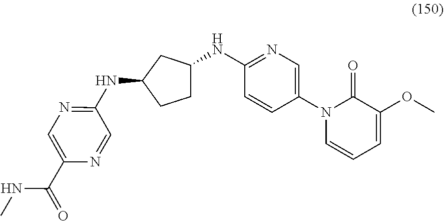

Methyl 5-(((1S,3S)-3-((3-methoxy-2-oxo-2H-[1,3′-bipyridin]-6′-yl)amino)cyclopentyl)ami-no)pyrazine-2-carboxylate compound i-89a (130 mg, 0.30 mmol) was added to methaneamine (30 wt % solution) in EtOH (15 mL) in a microwave vial. The vial was sealed and heated to 100° C. for 2 h in the microwave reactor. The solvent was removed under reduced pressure to afford the crude product. The crude product was purified by preparative HPLC (PrepMethod M, gradient: 18-35%) to give (42 mg, 32%) of the title compound as a pale-yellow solid. HRMS (ESI) m/z [M+H]⁺ calcd for C₂₂H₂₆N₇O₃: 436.2092, found: 436.2090 ¹H NMR (300 MHz, DMSO-d₆) δ 1.43-1.62 (2H, m), 1.92 (2H, t), 2.05-2.26 (2H, m), 2.76 (3H, d), 3.73 (3H, s), 4.30-4.45 (2H, m), 6.20 (1H, t), 6.54 (1H, d), 6.86 (1H, dd), 6.97 (1H, d), 7.17 (1H, dd), 7.39 (1H, dd), 7.80 (1H, br d), 7.85-7.89 (1H, m), 7.92 (1H, d), 8.17-8.35 (1H, m), 8.47-8.55 (1H, m).

### Example 162

### 6′-(((1S,3S)-3-((5-(Methylthio)pyrazin-2-yl)amino)cyclopentyl)amino)-2H-[1,3′-bipyridin]-2-one—compound 162

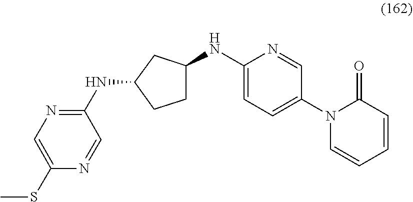 [SMILES: CSc1cnc(N[C@H]2CC[C@H](Nc3ccc(-n4ccccc4=O)cn3)C2)cn1]

2-Bromo-5-(methylthio)pyrazine (CAS Reg. No. 1049026-49-4) (100 mg, 0.49 mmol) was added to 6′-(((1S,3S)-3-aminocyclopentyl)amino)-2H-[1,3′-bipyridin]-2-one HCl compound i-1c (180 mg, 0.59 mmol), Pd-PEPPSI-IpentCl 2-methylpyridine (20.5 mg, 0.02 mmol) and K₂CO₃ (202 mg, 1.46 mmol) in 1,4-dioxane (10 mL) and the resulting mixture was stirred at 100° C. for 15 h under a nitrogen atmosphere. The reaction mixture was quenched with water (100 mL) and extracted with EtOAc (3×75 mL). The combined organic layers were dried over Na₂SO₄, filtered and evaporated and the residue purified by preparative TLC (MeOH:DCM=1:10) followed by preparative HPLC (PrepMethod E, gradient: 2-28%) to give the title compound (50 mg, 26%) as a white solid. HRMS (ESI) m/z [M+H]⁺ calcd for C₂₀H₂₃N₆OS: 395.1648, found: 395.1626. ¹H NMR (300 MHz, MeOH-d₄) δ 1.46-1.71 (2H, m), 2.00 (2H, t), 2.15-2.39 (2H, m), 2.45 (3H, s), 4.2-4.46 (2H, m), 6.47 (1H, td), 6.62 (2H, dd), 7.44 (1H, dd), 7.55-7.66 (2H, m), 7.83 (1H, d), 7.9-7.99 (2H, m).

### Example 163-164

Prepared as described for Example 162 using 6′-(((1S,3S)-3-aminocyclopentyl)amino)-2H-[1,3′-bipyridin]-2-one compound i-1c and the appropriate bromide. Cs₂CO₃ (3 eq) was used instead of K₂CO₃.

### Example 163

### 6′-(((1S,3S)-3-((5-(Ethylthio)pyrazin-2-yl)amino)cyclopentyl)amino)-2H-[1,3′-bipyridin]-2-one—compound 163

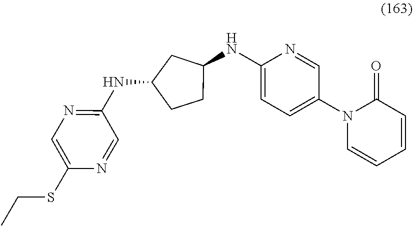 [SMILES: CCSc1cnc(N[C@H]2CC[C@H](Nc3ccc(-n4ccccc4=O)cn3)C2)cn1]

The reaction was performed using compound i-1c (418 mg, 1.22 mmol) and 2-bromo-5-(ethylthio)pyrazine (CAS Reg. No. 1310378-31-4; preparation described in WO2011068171A1 and WO2015017305A1) (400 mg, 1.83 mmol). The crude product was purified by preparative TLC (EtOAc) followed by preparative HPLC (PrepMethod E, gradient: 8-40%) to give the title compound (186 mg, 36%) as a white solid. HRMS (ESI) m/z [M+H]⁺ calcd for C₂₁H₂₅N60S: 409.1804, found: 409.1798. ¹H NMR (400 MHz, DMSO-d₆) 1.19 (3H, t), 1.43-1.57 (2H, m), 1.89 (2H, t), 2.08-2.22 (2H, m), 2.93 (2H, q), 4.19-4.38 (2H, m), 6.28 (1H, td), 6.45 (1H, d), 6.53 (1H, d), 6.96 (1H, d), 7.21 (1H, d), 7.40 (1H, dd), 7.43-7.53 (1H, m), 7.61 (1H, dd), 7.89 (1H, d), 7.93 (1H, d), 7.95 (1H, d).

### Example 164

### Methyl 2-((5-(((1S,3S)-3-((2-oxo-2H-[1,3′-bipyridin]-6′-yl)amino)cyclopentyl)amino)pyrazin-2-yl)thio)acetate—compound 164

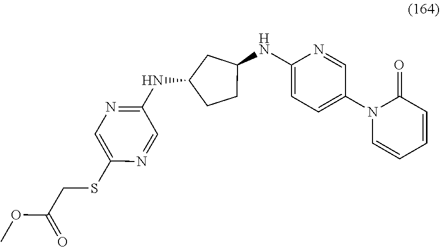 [SMILES: COC(=O)CSc1cnc(N[C@H]2CC[C@H](Nc3ccc(-n4ccccc4=O)cn3)C2)cn1]

The reaction was performed using compound i-1c (74 mg, 0.22 mmol) and methyl 2-((5-bromopyrazin-2-yl)thio)acetate compound i-99a (85 mg, 0.32 mmol). The crude product was purified by preparative TLC (EtOAc:PE=1:4) followed by preparative HPLC (PrepMethod E, gradient: 9-21%) to give the title compound (30 mg, 30%) as a white solid. HRMS (ESI) m/z [M+H]⁺ calcd for C₂₂H₂₅N₆O₃S: 453.1704, found: 453.1722. ¹H NMR (400 MHz, DMSO-d₆) 1.45-1.56 (2H, m), 1.89 (2H, t), 2.09-2.21 (2H, m), 3.60 (3H, s), 3.81 (2H, s), 4.21-4.40 (2H, m), 6.25-6.30 (1H, m), 6.43-6.46 (1H, m), 6.50-6.56 (1H, m), 6.96 (1H, d), 7.25-7.31 (1H, m), 7.40 (1H, dd), 7.44-7.53 (1H, m), 7.58-7.64 (1H, m), 7.86 (1H, d), 7.93 (1H, d), 7.98 (1H, d).

### Example 170

### rel-3-Methoxy-6′-(((1R,3R)-3-((5-methylpyrazin-2-yl)amino)cyclopentyl)amino)-2H-[1,3′-bipyridin]-2-one—compound 170

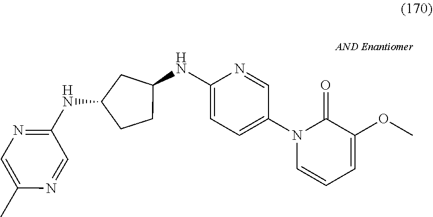

rel-(1R,3R)—N¹-(5-Methylpyrazin-2-yl)cyclopentane-1,3-diamine 3×TFA compound i-98b (0.878 g, 1.65 mmol), 6′-fluoro-3-methoxy-2H-[1,3′-bipyridin]-2-one compound i-97a (0.31 g, 1.41 mmol), DMSO (5 mL) and K₂CO₃ (1 g, 7.24 mmol) was added to a vial, the vial was capped (equipped with a thin needle through the septum) and the mixture was stirred and heated at 120° C. for 16 h in a pre-heated aluminium block. The mixture was cooled to rt and filtered. The filter cake was washed with MeOH and the combined filtrates were concentrated. The remaining DMSO was evaporated using a Biotage V10 instrument (high boiling method, 80° C.) and the obtained black residue was dissolved in DCM and washed with water. The phases were separated using a phase separator. The organic phase was concentrated under reduced pressure and the remaining residue purified by preparative HPLC (PrepMethod F, gradient: 15-60%) to give crude product after freeze drying. To the residue, still containing some water, was added MeCN and the solid was sonicated for 5 min, filtered off, washed with MeCN and dried under vacuum to give the title compound (0.235 g, 42%) as an off-white solid. HRMS (ESI) m/z [M+H]⁺ calcd for C₂₁H₂₅N₆O₂: 393.2034, found: 393.2026. ¹H NMR (500 MHz, DMSO-d₆) δ 1.39-1.57 (2H, m), 1.87 (2H, t), 2.1-2.19 (2H, m), 2.23 (3H, s), 3.72 (3H, s), 4.14-4.4 (2H, m), 6.19 (1H, t), 6.52 (1H, dd), 6.81-6.9 (2H, m), 6.93 (1H, d), 7.16 (1H, dd), 7.37 (1H, dd), 7.74-7.87 (2H, m), 7.90 (1H, dd).

### Example 171

### rel-3-Methoxy-6′-(((1R,3R)-3-((5-methylpyrazin-2-yl)amino)cyclopentyl)amino)-2H-[1,3′-bipyridin]-2-one, Isomer 1—compound 171

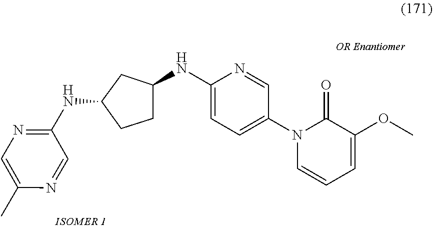

The enantiomers of rel-3-methoxy-6′-(((1R,3R)-3-((5-methylpyrazin-2-yl)amino)cyclo-pentyl)amino)-2H-[1,3′-bipyridin]-2-one Example 170 (0.215 g, 0.55 mmol) were separated by chiral chromatography on a CelluCoat column (5 μm, 250×30 mm ID) eluted with 30% MeOH/DEA (100/20 mM) in CO₂, 130 bar, at a flow rate of 100 mL/min and detected at 250 nm. The first eluted compound was collected and evaporated to give crude Isomer 1 (31 mg); for the second eluted compound see Example 172 (Isomer 2). The major amount of crude Isomer 1 (24 mg) was further purified by chiral chromatography on a Chiralpak-IBN (5 μm, 250×30 mm ID) eluted with MeOH/DEA (B) (100/20 mM) in CO₂, gradient: 40% B for 1 min, 40-45% B for 3 min then 45% B for 11 min, 120 bar, at a flow rate of 100 mL/min and detected at 248 nm. Isomer 1 was collected, dissolved in DCM and washed with water. The organic phase was passed through a phase separator and concentrated. The residue was dissolved in MeCN and water and freeze dried overnight to give Isomer 1, Example 171 (18.9 mg, 79%) as a white solid. HRMS (ESI) m/z [M+H]⁺ calcd for C₂₁H₂₅N₆O₂: 393.2034, found: 393.2042. ¹H NMR (500 MHz, MeOH-d₄) δ 1.51-1.69 (2H, m), 1.99 (2H, t), 2.21-2.37 (5H, m), 3.85 (3H, s), 4.24-4.41 (2H, m), 6.39 (1H, t), 6.61 (1H, d), 7.00 (1H, dd), 7.16 (1H, dd), 7.44 (1H, dd), 7.79 (1H, d), 7.81-7.86 (1H, m), 7.94 (1H, d).

### Example 172

### rel-3-Methoxy-6′-(((1R,3R)-3-((5-methylpyrazin-2-yl)amino)cyclopentyl)amino)-2H-[1,3′-bipyridin]-2-one, Isomer 2—compound 172

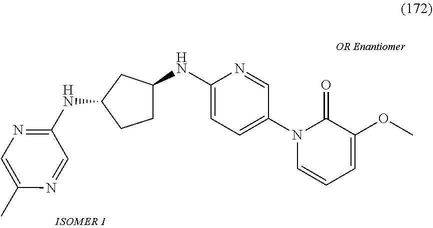

The second eluted compound was collected and evaporated to give the title compound Isomer 2, Example 172 (34 mg, 15%). HRMS (ESI) m/z [M+H]⁺ calcd for C₂₁1H₂₅N₆O₂: 393.2034, found: 393.2034. ¹H NMR (500 MHz, MeOH-d₄) δ 1.52-1.7 (2H, m), 1.99 (2H, t), 2.2-2.4 (5H, m), 3.85 (3H, s), 4.2-4.42 (2H, m), 6.39 (1H, t), 6.61 (1H, d), 6.99 (1H, dd), 7.16 (1H, dd), 7.44 (1H, dd), 7.80 (2H, d), 7.94 (1H, d).

### Example 176

### 6′-(((1S,3S)-3-((5,6-Dimethylpyrazin-2-yl)amino)cyclopentyl)amino)-5-(1H-tetrazol-5-yl)-2H-[1,3′-bipyridin]-2-one—compound 176

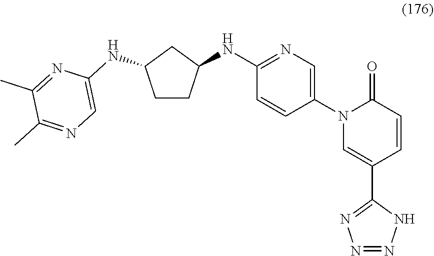 [SMILES: Cc1ncc(N[C@H]2CC[C@H](Nc3ccc(-n4cc(-c5nnn[nH]5)ccc4=O)cn3)C2)nc1C]

6′-(((1S,3S)-3-((5,6-dimethylpyrazin-2-yl)amino)cyclopentyl)amino)-5-(1-(4-methoxybenzyl)-1H-tetrazol-5-yl)-2H-[1,3′-bipyridin]-2-one compound i-93a (100 mg, 0.18 mmol) was added to TFA (5 mL) at rt and the resulting solution was stirred at 80° C. for 3 h. The solvent was removed under reduced pressure and the crude product was purified by preparative HPLC (PrepMethod D, gradient: 5-30%) to afford (45 mg, 57%) of the title compound as a white solid. HRMS (ESI) m/z [M+H]⁺ calcd for C₂₂H₂₅N₁₀O: 445.2208, found: 445.2184. ¹H NMR (300 MHz, DMSO-d₆) δ ppm 1.42-1.57 (2H, m), 1.90 (2H, brt), 2.14 (2H, d), 2.26 (3H, s), 2.27 (3H, s), 4.21-4.41 (2H, m), 6.52-6.73 (3H, m), 7.01 (1H, d), 7.49 (1H, dd), 7.62 (1H, s), 8.01-8.08 (2H, m), 8.21 (1H, d).

### Example 177

### 6′-(((1S,3S)-3-((5-Methylpyrazin-2-yl)amino)cyclopentyl)amino)-5-(1H-tetrazol-5-yl)-2H-[1,3′-bipyridin]-2-one—compound 177

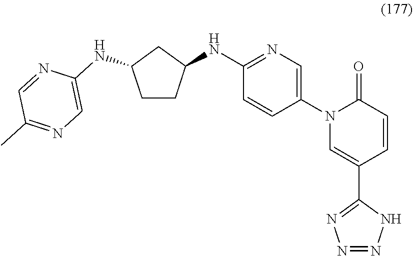 [SMILES: Cc1cnc(N[C@H]2CC[C@H](Nc3ccc(-n4cc(-c5nnn[nH]5)ccc4=O)cn3)C2)cn1]

5-(1-(4-Methoxybenzyl)-1H-tetrazol-5-yl)-6′-(((1S,3S)-3-((5-methylpyrazin-2-yl)amino)cyclopentyl)amino)-2H-[1,3′-bipyridin]-2-one compound i-94a (65 mg, 0.12 mmol) was added to TFA (3 mL) at rt and the resulting solution was stirred at 80° C. for 2 h. The solvent was removed under reduced pressure and the crude product was purified by preparative HPLC (PrepMethod D, gradient: 5-30%) to afford (40 mg, 79%) of the title compound as a white solid. HRMS (ESI) m/z [M+H]⁺ calcd for C₂₁H₂₃N₁₀O: 431.2050, found: 431.2026. ¹H NMR (300 MHz, DMSO-d₆) δ ppm 1.43-1.59 (2H, m), 1.89 (2H, brt), 2.10-2.22 (2H, m), 2.25 (3H, s), 4.22-4.4 (2H, m), 6.53-6.66 (2H, dd), 6.87 (1H, d), 7.01 (1H, d), 7.49 (1H, dd), 7.79-7.87 (2H, m), 8.01-8.08 (2H, m), 8.18 (1H, d).

### Example 178

### 6′-(((1S,3S)-3-((5-(Difluoromethoxy)pyrazin-2-yl)amino)cyclopentyl)amino)-5-(1H-tetrazol-5-yl)-2H-[1,3′-bipyridin]-2-one—compound 178

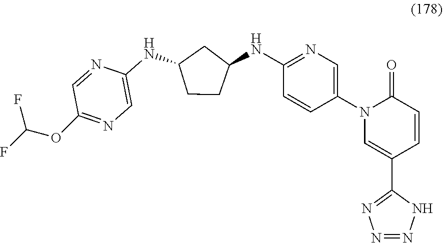 [SMILES: O=c1ccc(-c2nnn[nH]2)cn1-c1ccc(N[C@H]2CC[C@H](Nc3cnc(OC(F)F)cn3)C2)nc1]

6′-(((1S,3S)-3-((5-(difluoromethoxy)pyrazin-2-yl)amino)cyclopentyl)amino)-5-(1-(4-methoxybenzyl)-1H-tetrazol-5-yl)-2H-[1,3′-bipyridin]-2-one compound i-95a (110 mg, 0.18 mmol) was added to TFA (5 mL) at rt and the resulting solution was stirred at 80° C. for 2 h. The solvent was removed under reduced pressure and the crude product was purified by preparative HPLC (PrepMethod D, gradient: 11-31%) to afford (50 mg, 57%) of the title compound as a white solid. HRMS (ESI) m/z [M+H]⁺ calcd for C₂₁H₂₁F₂N₁₀O₂: 483.1812, found: 483.1818. ¹H NMR (300 MHz, DMSO-d₆) δ ppm 1.43-1.66 (2H, m), 1.91 (2H, t), 2.07-2.25 (2H, m), 4.2-4.44 (3H, m), 6.52-6.67 (2H, m), 7.02 (1H, d), 7.16 (1H, d), 7.33 (1H, t, CHF₂), 7.44-7.54 (1H, m), 7.61 (1H, s), 7.91 (1H, s), 8.04 (2H, d), 8.19 (1H, d).

### Example 179

### 6′-(((1S,3S)-3-((5-Chloropyrazin-2-yl)amino)cyclopentyl)amino)-5-(1H-tetrazol-5-yl)-2H-[1,3′-bipyridin]-2-one—compound 179

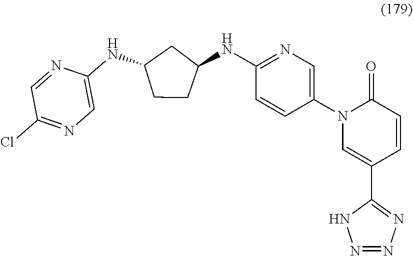 [SMILES: O=c1ccc(-c2nnn[nH]2)cn1-c1ccc(N[C@H]2CC[C@H](Nc3cnc(Cl)cn3)C2)nc1]

6′-(((1S,3S)-3-((5-chloropyrazin-2-yl)amino)cyclopentyl)amino)-5-(1-(4-methoxybenzyl)-1H-tetrazol-5-yl)-2H-[1,3′-bipyridin]-2-one compound i-96c (40 mg, 0.07 mmol) was treated with TFA (1 mL) and it was stirred at rt for 3 h. The solvent was removed under reduced pressure and the crude product was purified by preparative HPLC (PrepMethod M, gradient: 13-32%) to afford (10 mg, 32%) of the title compound as a white solid. HRMS (ESI) m/z [M+H]⁺ calcd for C₂₀H₂₀ClN₁₀O: 451.1504, found: 451.1486. ¹H NMR (300 MHz, DMSO-d₆) δ ppm 1.42-1.61 (2H, m), 1.91 (2H, t), 2.09-2.22 (2H, m), 4.20-4.45 (2H, m), 6.55 (1H, d), 6.60 (1H, d), 7.02 (1H, d), 7.40-7.54 (2H, m), 7.73 (1H, d), 8.0-8.1 (3H, m), 8.14 (1H, d).

### Example 203

### 3-(1-Methyl-1H-pyrazol-4-yl)-6′-(((1S,3S)-3-((5-methylpyrazin-2-yl)amino)cyclopentyl)amino)-2H-[1,3′-bipyridin]-2-one—compound 203

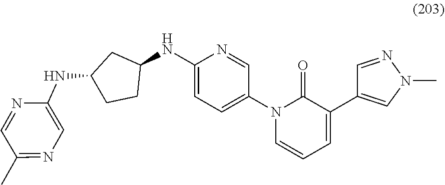 [SMILES: Cc1cnc(N[C@H]2CC[C@H](Nc3ccc(-n4cccc(-c5cnn(C)c5)c4=O)cn3)C2)cn1]

3-Chloro-6′-(((1S,3S)-3-((5-methylpyrazin-2-yl)amino)cyclopentyl)amino)-2H-[1,3′-bipyridin]-2-one Example 47 (250 mg, 0.63 mmol) was added to (1-methyl-1H-pyrazol-4-yl)boronic acid (CAS Reg. No. 847818-55-7) (79 mg, 0.63 mmol), Cs₂CO₃ (205 mg, 0.63 mmol), XPhos (300 mg, 0.63 mmol) and XPhos Pd G3 (533 mg, 0.63 mmol) in 1,4-dioxane (8 mL) and water (2 mL) at rt and the resulting solution was stirred at 100° C. for 18 h under nitrogen. The reaction mixture was diluted with EtOAc (75 mL) and washed sequentially with water (3×50 mL) and brine (3×25 mL). The organic layer was dried (Na₂SO₄), filtered and evaporated and the residue was first purified by preparative TLC (MeOH:DCM=1:20) followed by preparative HPLC (PrepMethod X, gradient: 18-38%) to afford (2 mg, 10%) of the title compound as a white solid. HRMS (ESI) m/z [M+H]⁺ calcd for C₂₄H₂₇N₈O: 443.2302, found: 443.2338. ¹H NMR (300 MHz, DMSO-d₆) δ ppm 1.41-1.60 (2H, m), 1.89 (2H, t), 2.08-2.20 (2H, m), 2.25 (3H, s), 3.85 (3H, s), 4.20-4.41 (2H, m), 6.36 (1H, t), 6.55 (1H, d), 6.87 (1H, d), 6.95 (1H, d), 7.44 (1H, dd), 7.53 (1H, dd), 7.79-7.89 (3H, m), 7.97 (1H, d), 8.01 (1H, s), 8.31 (1H, s).

### Example 204

### 6′-(((1S,3S)-3-((5-Methylpyrazin-2-yl)amino)cyclopentyl)amino)-3-(1H-pyrazol-4-yl)-2H-[1,3′-bipyridin]-2-one—compound 204

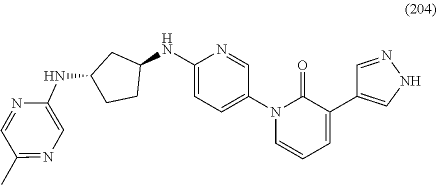 [SMILES: Cc1cnc(N[C@H]2CC[C@H](Nc3ccc(-n4cccc(-c5cn[nH]c5)c4=O)cn3)C2)cn1]

Cs₂CO₃ (181 mg, 0.55 mmol) was added to 3-chloro-6′-(((1S,3S)-3-((5-methylpyrazin-2-yl)amino)cyclopentyl)amino)-2H-[1,3′-bipyridin]-2-one Example 47 (220 mg, 0.55 mmol), (1-(tert-butoxycarbonyl)-1H-pyrazol-4-yl)boronic acid (CAS Reg. No. 1188405-87-9) (118 mg, 0.55 mmol), XPhos (264 mg, 0.55 mmol) and XPhos Pd G3 (469 mg, 0.55 mmol) in 1,4-dioxane (8 mL) and water (2 mL) at rt and the resulting suspension was stirred at 100° C. for 18 h under nitrogen. The reaction mixture was diluted with EtOAc (75 mL) and washed sequentially with water (3×50 mL) and brine (3×25 mL). The organic layer was dried (Na₂SO₄), filtered and evaporated and the residue was first purified by preparative TLC (MeOH:DCM=1:20) and then by preparative HPLC (PrepMethod Z6, gradient: 17-34%) to afford (89 mg, 38%) of the title compound as a white solid. HRMS (ESI) m/z [M+H]⁺ calcd for C₂₃H₂₅N₈O: 429.2146, found: 429.2144. ¹H NMR (300 MHz, DMSO-d₆) δ ppm 1.37-1.60 (2H, m), 1.89 (2H, t), 2.02-2.21 (2H, m), 2.25 (3H, s), 4.2-4.4 (2H, m), 6.36 (1H, t), 6.54 (1H, d), 6.86 (1H, d), 6.93 (1H, d), 7.43 (1H, dd), 7.52 (1H, dd), 7.77-7.85 (2H, m), 7.89 (1H, dd), 7.96 (1H, d), 8.08 (1H, s), 8.34 (1H, s), 12.86 (1H, s).

### Example 205

### 6′-(((1S,3S)-3-((5-Methylpyrazin-2-yl)amino)cyclopentyl)amino)-3-(1H-1,2,3-triazol-4-yl)-2H-[1,3′-bipyridin]-2-one—compound 205

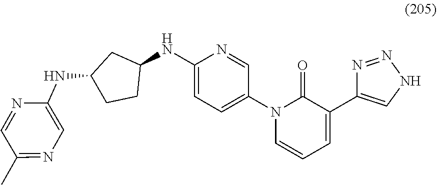 [SMILES: Cc1cnc(N[C@H]2CC[C@H](Nc3ccc(-n4cccc(-c5c[nH]nn5)c4=O)cn3)C2)cn1]

4 M HCl in MeOH (2.5 mL, 10 mmol) was added to 6′-(((1S,3S)-3-((5-methylpyrazin-2-yl)amino)cyclopentyl)amino)-3-(2-(tetrahydro-2H-pyran-2-yl)-2H-1,2,3-triazol-4-yl)-2H-[1,3′-bipyridin]-2-one compound i-68b (150 mg, 0.29 mmol) in MeOH (10 mL) at 15° C. and the resulting mixture was stirred at 60° C. for 2 h. The solvent was removed under reduced pressure to afford crude product which was purified by preparative HPLC (PrepMethod D, gradient: 2-28%) to afford (27 mg, 22%) of the title compound as a white solid. HRMS (ESI) m/z [M+H]⁺ calcd for C₂₂H₂₄N₉O: 430.2098, found: 430.2116. ¹H NMR (300 MHz, DMSO-d₆) δ ppm 1.4-1.6 (2H, m), 1.89 (2H, t), 2.1-2.2 (2H, m), 2.25 (3H, s), 4.2-4.4 (2H, m), 6.48 (1H, t), 6.56 (1H, d), 6.86 (1H, d), 6.97 (1H, d), 7.48 (1H, dd), 7.71 (1H, d), 7.79-7.86 (2H, m), 7.99 (1H, d), 8.27 (1H, dd), 8.37 (1H, s).

### Example 206

### 5-(1-Methyl-1H-pyrazol-4-yl)-6′-(((1S,3S)-3-((5-methylpyrazin-2-yl)amino)cyclopentyl)amino)-2H-[1,3′-bipyridin]-2-one—compound 206

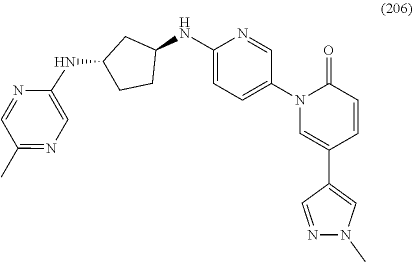

6′-Chloro-5-(1-methyl-1H-pyrazol-4-yl)-2H-[1,3′-bipyridin]-2-one compound i-67b (100 mg, 0.35 mmol), (1S,3S)—N¹-(5-methylpyrazin-2-yl)cyclopentane-1,3-diamine×2HCl compound i-31b (92 mg, 0.35 mmol), Cs₂CO₃ (568 mg, 1.74 mmol) and Pd-PEPPSI-IpentCl 2-methylpyridine (147 mg, 0.17 mmol) in DMF (8 mL) was stirred under an atmosphere of nitrogen at 100° C. for 18 h. The reaction mixture was filtered through Celite and washed with EtOAc (40 mL). The combined filtrates were concentrated under reduced pressure and the residue was purified by preparative HPLC (PrepMethod 0, gradient: 24-54%) to afford (20 mg, 13%) of the title compound as a pale-yellow solid. HRMS (ESI) m/z [M+H]⁺ calcd for C₂₄H₂₇N₈O: 443.2302, found: 443.2316. ¹H NMR (300 MHz, DMSO-d₆) δ ppm 1.45-1.6 (2H, m), 1.89 (2H, t), 2.1-2.2 (2H, m), 2.25 (3H, s), 3.82 (3H, s), 4.21-4.40 (2H, m), 6.53 (2H, t), 6.86 (1H, d), 6.94 (1H, d), 7.45 (1H, dd), 7.70-7.85 (4H, m), 7.88 (1H, d), 7.95-8.06 (2H, m).

### Example 207

### 6′-(((1S,3S)-3-((5-Methylpyrazin-2-yl)amino)cyclopentyl)amino)-5-(1H-pyrazol-4-yl)-2H-[1,3′-bipyridin]-2-one—compound 207

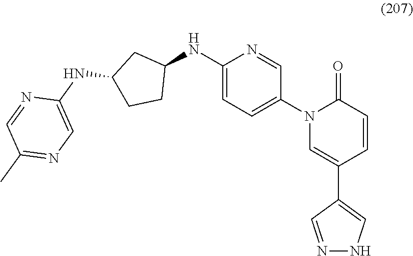 [SMILES: Cc1cnc(N[C@H]2CC[C@H](Nc3ccc(-n4cc(-c5cn[nH]c5)ccc4=O)cn3)C2)cn1]

5-(1-(4-Methoxybenzyl)-1H-pyrazol-4-yl)-6′-(((1S,3S)-3-((5-methylpyrazin-2-yl)amino)cyclopentyl)amino)-2H-[1,3′-bipyridin]-2-one compound i-50e (130 mg, 0.24 mmol) was treated with TFA (7 mL) at rt and it was stirred at 80° C. for 5 h. The reaction mixture was evaporated to afford crude product which was purified by preparative HPLC (PrepMethod Z7, gradient: 16-36%) to afford (26 mg, 25%) of the title compound as a white solid. HRMS (ESI) m/z [M+H]⁺ calcd for C₂₃H₂₅N₈O: 429.2146, found: 429.2172. ¹H NMR (300 MHz, DMSO-d₆) δ ppm 1.45-1.6 (2H, m), 1.89 (2H, t), 2.06-2.20 (2H, m), 2.25 (3H, s), 4.2-4.4 (2H, m), 6.50-6.56 (2H, m), 6.86 (1H, d), 6.94 (1H, d), 7.45 (1H, dd), 7.77-7.86 (3H, m), 7.91 (1H, d), 7.99 (1H, d), 8.09 (1H, s), 12.85 (1H, s).

### Example 209

### 6′-(((1S,3S)-3-((5-Methylpyrazin-2-yl)amino)cyclopentyl)amino)-5-(1H-1,2,3-triazol-4-yl)-2H-[1,3′-bipyridin]-2-one—compound 209

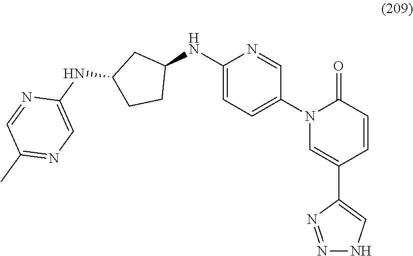 [SMILES: Cc1cnc(N[C@H]2CC[C@H](Nc3ccc(-n4cc(-c5c[nH]nn5)ccc4=O)cn3)C2)cn1]

5-(1-(4-Methoxybenzyl)-1H-1,2,3-triazol-4-yl)-6′-(((1S,3S)-3-((5-methylpyrazin-2-yl)amino)cyclopentyl)amino)-2H-[1,3′-bipyridin]-2-one compound i-49d (10 mg, 0.02 mmol) was added into TFA (2.0 mL) at rt and the resulting solution was stirred at 80° C. for 2 h. The solvent was removed under reduced pressure to afford the crude product which was purified by preparative HPLC (PrepMethod 0, gradient: 5-27%) to afford (1.5 mg, 19%) of the title compound as a white solid. HRMS (ESI) m/z [M+H]⁺ calcd for C₂₂H₂₄N₉O: 430.2098, found: 430.2116. ¹H NMR (300 MHz, Methanol-d₄) δ ppm 1.55-1.73 (2H, m), 1.97-2.07 (2H, m), 2.2-2.32 (2H, m), 2.34 (4H, s), 4.28-4.44 (2H, m), 6.61-6.71 (1H, m), 6.74 (1H, dd), 7.53 (1H, dd), 7.81 (1H, d), 7.85 (1H, s), 8.01-8.16 (4H, m).

### Example 215

### 1-Methyl-3-(6-(((1S,3S)-3-((5-methylpyrazin-2-yl)amino)cyclopentyl)amino)pyridin-3-yl)-2-oxo-2,3-dihydro-1H-benzo[d]imidazole-5-carbonitrile—compound 215

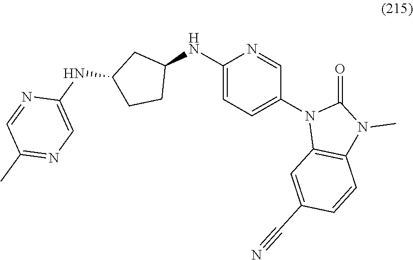

In a slight variation of GM2 (1S,3S)—N¹-(5-methylpyrazin-2-yl)cyclopentane-1,3-diamine 2HCl compound i-31b (100 mg, 0.38 mmol), 3-(6-chloropyridin-3-yl)-1-methyl-2-oxo-2,3-dihydro-1H-benzo[d]imidazole-5-carbonitrile compound i-60d (118 mg, 0.41 mmol), Pd-PEPPSI-IpentCl 2-methylpyridine (16 mg, 0.02 mmol) and Cs₂CO₃ (369 mg, 1.13 mmol) were reacted in DMF (15 mL) at 90° C. for 18 h under nitrogen. Upon aqueous work-up (EtOAc), preparative TLC (EtOAc) followed by preparative HPLC (PrepMethod 0, gradient: 30-47%) the title compound (29 mg, 17%) was obtained as a white solid. HRMS (ESI) m/z [M+H]⁺ calcd for C₂₄H₂₅N₈O: 441.2146, found: 441.2168. ¹H NMR (300 MHz, DMSO-d₆) δ ppm 1.42-1.58 (2H, m), 1.87-1.92 (2H, m), 2.11-2.19 (2H, m), 2.24 (3H, s), 3.43 (3H, s), 4.21-4.30 (1H, m), 4.32-4.41 (1H, m), 6.61 (1H, d), 6.86 (1H, d), 7.03 (1H, d), 7.30 (1H, d), 7.42 (1H, d), 7.47 (1H, dd), 7.60 (1H, dd), 7.81-7.82 (2H, m), 8.06 (1H, d).

### Example 216

### 3-Methyl-1-(6-(((1S,3S)-3-((5-methylpyrazin-2-yl)amino)cyclopentyl)amino)pyridin-3-yl)-2-oxo-2,3-dihydro-1H-benzo[d]imidazole-5-carbonitrile—compound 216

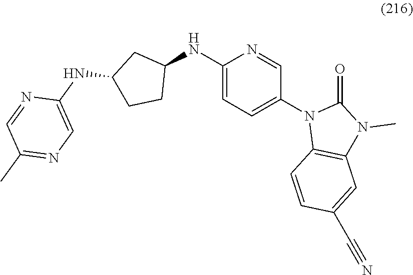

According to GM2 (1S,3S)—N¹-(5-methylpyrazin-2-yl)cyclopentane-1,3-diamine 2HCl compound i-31b (110 mg, 0.41 mmol), 1-(6-chloropyridin-3-yl)-3-methyl-2-oxo-2,3-dihydro-1H-benzo[d]imidazole-5-carbonitrile compound i-61d (70 mg, 0.25 mmol), Pd-PEPPSI-IpentCl 2-methylpyridine (17 mg, 0.02 mmol) and Cs₂CO₃ (405 mg, 1.24 mmol) were reacted in 1,4-dioxane (15 mL) at 100° C. for 18 h under nitrogen. Upon aqueous work-up (EtOAc), preparative TLC (EtOAc) followed by preparative HPLC (PrepMethod X, gradient: 30-50%) the title compound (34 mg, 18%) was obtained as a white solid. HRMS (ESI) m/z [M+H]⁺ calcd for C₂₄H₂₅N₈O: 441.2146, found: 441.2134. ¹H NMR (300 MHz, DMSO-d₆) δ ppm 1.42-1.57 (2H, m), 1.84-1.93 (2H, m), 2.10-2.19 (2H, m), 2.24 (3H, s), 3.41 (3H, s), 4.20-4.29 (1H, m), 4.32-4.40 (1H, m), 6.61 (1H, d), 6.86 (1H, d), 7.01 (1H, d), 7.05 (1H, d), 7.44-7.50 (2H, m), 7.78-7.82 (3H, m), 8.05 (1H, d).

### Example 218

### 1-Methyl-3-(6-(((1S,3S)-3-((5-methylpyrazin-2-yl)amino)cyclopentyl)amino)pyridin-3-yl)-2-oxo-2,3-dihydro-1H-imidazo[4,5-b]pyridine-6-carbonitrile—compound 218

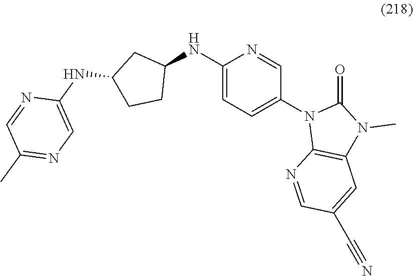

According to GM2 (1S,3S)—N¹-(5-methylpyrazin-2-yl)cyclopentane-1,3-diamine compound i-31b (202 mg, 1.05 mmol), 3-(6-chloropyridin-3-yl)-1-methyl-2-oxo-2,3-dihydro-1H-imidazo[4,5-b]pyridine-6-carbonitrile compound i-82d (150 mg, 0.53 mmol), Pd-PEPPSI-IpentCl 2-methylpyridine (44 mg, 0.05 mmol) and Cs₂CO₃ (513 mg, 1.58 mmol) were reacted in 1,4-dioxane (8 mL) at 100° C. for 15 h under nitrogen. Upon filtration, preparative TLC (DCM:MeOH=10:1) followed by preparative HPLC (PrepMethod E, gradient: 4-30%) the title compound (31 mg, 13%) was obtained as a white solid. HRMS (ESI) m/z [M+H]⁺ calcd for C₂₃H₂₄N₉O: 442.2098, found: 442.2128. ¹H NMR (300 MHz, DMSO-d₆) δ ppm 1.42-1.57 (2H, m), 1.86-1.91 (2H, m), 2.10-2.19 (2H, m), 2.24 (3H, s), 3.42 (3H, s), 4.22-4.28 (1H, m), 4.30-4.39 (1H, m), 6.59 (1H, d), 6.86 (1H, d), 7.00 (1H, d), 7.49 (1H, dd), 7.81-7.82 (2H, m), 8.07-8.10 (2H, m), 8.42 (1H, dd).

### Example 226

### 4-Methoxy-2-(6-(((1S,3S)-3-((5-methylpyrazin-2-yl)amino)cyclopentyl)amino)pyridin-3-yl)pyridazin-3 (2H)-one—compound 226

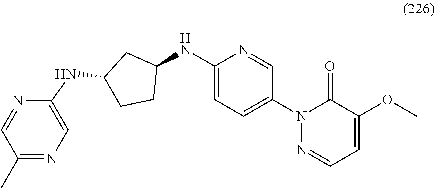 [SMILES: COc1ccnn(-c2ccc(N[C@H]3CC[C@H](Nc4cnc(C)cn4)C3)nc2)c1=O]

rel-(1R,2R)—N¹,N²-Dimethylcyclohexane-1,2-diamine (43.2 mg, 0.30 mmol) was added to (1S,3S)—N-(5-iodopyridin-2-yl)-N3-(5-methylpyrazin-2-yl)cyclopentane-1,3-diamine compound i-32a (120 mg, 0.30 mmol), 4-methoxypyridazin-3 (2H)-one (CAS Reg. No. 38732-07-9) (77 mg, 0.61 mmol), Cu(I)I (57.8 mg, 0.30 mmol) and K₂CO₃ (126 mg, 0.91 mmol) in DMF (10 mL) at 20° C. The resulting mixture was stirred at 100° C. for 15 h under a nitrogen atmosphere. The reaction mixture was concentrated, diluted with EtOAc (250 mL) and washed sequentially with sat. brine (4×50 mL). The organic layer was dried over Na₂SO₄, filtered and evaporated to afford crude product. The residue was purified by preparative TLC (MeOH:DCM=1:15) followed by preparative HPLC (PrepMethod D, gradient: 12-33%) to give the title compound (23 mg, 19%) as a white solid. HRMS (ESI) m/z [M+H]⁺ calcd for C₂₀H₂₄N₇O₂: 394.1986, found: 394.2006. ¹H NMR (400 MHz, DMSO-d₆) δ ppm 1.41-1.58 (2H, m), 1.88 (2H, t), 2.08-2.22 (2H, m), 2.24 (3H, s), 3.85 (3H, s), 4.2-4.4 (2H, m), 6.52 (1H, d), 6.82 (1H, d), 6.86 (1H, d), 6.93 (1H, d), 7.48 (1H, dd), 7.78-7.84 (2H, m), 7.90 (1H, d), 8.07 (1H, d).

### Example 227

### 4-Methyl-2-(6-(((1S,3S)-3-((5-methylpyrazin-2-yl)amino)cyclopentyl)amino)pyridin-3-yl)pyridazin-3 (2H)-one—compound 227

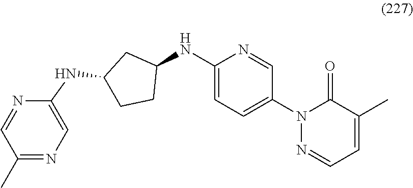 [SMILES: Cc1cnc(N[C@H]2CC[C@H](Nc3ccc(-n4nccc(C)c4=O)cn3)C2)cn1]

Cs₂CO₃ (371 mg, 1.14 mmol) was added to (1S,3S)—N¹-(5-iodopyridin-2-yl)-NV-(5-methylpyrazin-2-yl)cyclopentane-1,3-diamine compound i-32a (150 mg, 0.38 mmol), 4-methylpyridazin-3 (2H-one (CAS Reg. No. 33471-40-8) (84 mg, 0.76 mmol), Cu(I)I (36.1 mg, 0.19 mmol) and N¹,N²-dimethylethane-1,2-diamine (16.73 mg, 0.19 mmol) in DMF (5 mL) at 20° C. under a nitrogen atmosphere. The resulting suspension was stirred at 100° C. for 16 h. The reaction mixture was poured into water (100 mL) and extracted with EtOAc (2×100 mL). The organic layers were combined and washed with sat. brine (3×50 mL), dried over Na₂SO₄, filtered and evaporated. The residue was purified by preparative TLC (7 M NH₃ in MeOH:DCM=1:20) followed by preparative HPLC (PrepMethod X, gradient: 14-42%) to give the title compound (25.9 mg, 18%) as a yellow solid. HRMS (ESI) m/z [M+H]⁺ calcd for C₂₀H₂₄N₇O: 378.2036, found: 378.2058. ¹H NMR (300 MHz, DMSO-d₆) δ ppm 1.39-1.61 (2H, m), 1.88 (2H, t), 2.07-2.21 (5H, m), 2.24 (3H, s), 4.17-4.44 (2H, m), 6.52 (1H, d), 6.90 (2H, dd), 7.35 (1H, dd), 7.50 (1H, dd), 7.77-7.86 (2H, m), 7.90 (1H, d), 8.09 (1H, d).

### Example 231

### 6-Methyl-2-(6-(((1S,3S)-3-((5-methylpyrazin-2-yl)amino)cyclopentyl)amino)pyridin-3-yl)pyridazin-3 (2H)-one—compound 231

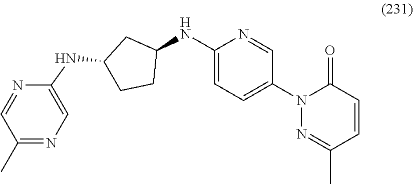 [SMILES: Cc1cnc(N[C@H]2CC[C@H](Nc3ccc(-n4nc(C)ccc4=O)cn3)C2)cn1]

Cs₂CO₃ (213 mg, 0.65 mmol) was added to 2-(6-(((1S,3S)-3-aminocyclopentyl)amino)pyridin-3-yl)-6-methylpyridazin-3 (2H)-one 3HCl compound i-69b (86 mg, 0.22 mmol), 2-bromo-5-methylpyrazine (CAS Reg. No. 98006-90-7) (75 mg, 0.44 mmol) and Pd-PEPPSI-IpentCl 2-methylpyridine (9.2 mg, 10.9 μmol) in 1,4-dioxane (5 mL) at 20° C. The resulting mixture was stirred at 100° C. for 15 h under a nitrogen atmosphere. The reaction mixture was diluted with water (20 mL) and extracted with EtOAc (3×50 mL). The organic layers were combined, dried over Na₂SO₄, filtered and evaporated. The residue was purified by preparative TLC (MeOH:DCM=1:20) followed by preparative HPLC (PrepMethod X, gradient: 17-44%) to give the title compound (55 mg, 67%) as a yellow solid. HRMS (ESI) m/z [M+H]⁺ calcd for C₂₀H₂₄N₇O: 378.2036, found: 378.2036. ¹H NMR (300 MHz, DMSO-d₆) δ ppm 1.42-1.56 (2H, m), 1.88 (2H, t), 2.10-2.20 (2H, m), 2.24 (3H, s), 2.30 (3H, s), 4.20-4.42 (2H, m), 6.52 (1H, d), 6.85-7.00 (3H, m), 7.39 (1H, d), 7.49 (1H, dd), 7.79-7.85 (2H, m), 8.08 (1H, d).

### Example 232

### 4-Chloro-2-(6-(((1S,3S)-3-((5-methylpyrazin-2-yl)amino)cyclopentyl)amino)pyridin-3-yl)pyridazin-3 (2H)-one—compound 232

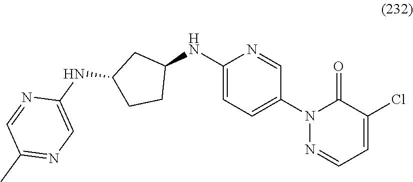 [SMILES: Cc1cnc(N[C@H]2CC[C@H](Nc3ccc(-n4nccc(Cl)c4=O)cn3)C2)cn1]

2-(6-(((1S,3S)-3-Aminocyclopentyl)amino)pyridin-3-yl)-4-chloropyridazin-3 (2H)-one compound i-70b (150 mg, 0.49 mmol) was added to 2-bromo-5-methylpyrazine (CAS Reg. No. 98006-90-7) (170 mg, 0.98 mmol), Pd-PEPPSI-IpentCl 2-methylpyridine (41.3 mg, 0.05 mmol) and Cs₂CO₃ (799 mg, 2.45 mmol) in 1,4-dioxane (15 mL) at 25° C. The resulting solution was stirred at 100° C. for 18 h under a nitrogen atmosphere. The reaction mixture was concentrated, diluted with EtOAc (250 mL) and washed sequentially with sat. brine (3×75 mL). The organic layer was dried over Na₂SO₄, filtered and evaporated. The residue was purified by preparative TLC (MeOH:DCM=1:12) followed by preparative HPLC (PrepMethod O, gradient: 18-38%) to give the title compound (15 mg, 8%) as a white solid. HRMS (ESI) m/z [M+H]⁺ calcd for C₁₉H₂₁ClN₇O: 398.1490, found: 398.1488. ¹H NMR (300 MHz, DMSO-d₆) δ ppm 1.41-1.6 (2H, m), 1.88 (2H, t), 2.08-2.21 (2H, m), 2.24 (3H, s), 4.16-4.43 (2H, m), 6.53 (1H, d), 6.86 (1H, d), 7.02 (1H, d), 7.53 (1H, dd), 7.78-7.87 (3H, m), 8.00 (1H, d), 8.12 (1H, d).

### Example 233

### 6-Chloro-2-(6-(((1S,3S)-3-((5-methylpyrazin-2-yl)amino)cyclopentyl)amino)pyridin-3-yl)pyridazin-3 (2H)-one—compound 233

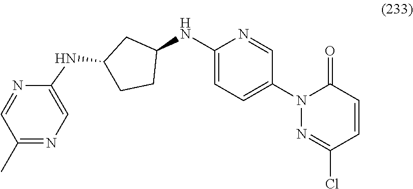 [SMILES: Cc1cnc(N[C@H]2CC[C@H](Nc3ccc(-n4nc(Cl)ccc4=O)cn3)C2)cn1]

Pd-PEPPSI-IpentCl 2-methylpyridine (13.8 mg, 0.02 mmol) was added to 2-bromo-5-methylpyrazine (CAS Reg. No. 98006-90-7) (113 mg, 0.65 mmol), 2-(6-(((1S,3S)-3-aminocyclopentyl)amino)pyridin-3-yl)-6-chloropyridazin-3 (2H)-one compound i-71 b (100 mg, 0.33 mmol) and Cs₂CO₃ (320 mg, 0.98 mmol) in 1,4-dioxane (10 mL) at 25° C. The resulting mixture was stirred at 100° C. for 15 h. The reaction mixture was concentrated, diluted with EtOAc (150 mL) and washed sequentially with sat. brine (3×50 mL). The organic layer was dried over Na₂SO₄, filtered and evaporated and the residue purified by preparative TLC (MeOH DCM=1:20) followed by preparative HPLC (SFC-A, gradient: 5-30%) to give the title compound (21 mg, 16%) as a yellow solid. HRMS (ESI) m/z [M+H]⁺ calcd for C₁₉H₂₁ClN₇O: 398.1490, found: 398.1520. ¹H NMR (300 MHz, DMSO-d₆) δ ppm 1.40-1.55 (2H, m), 1.88 (2H, t), 2.01-2.20 (2H, m), 2.24 (3H, s), 3.97-4.56 (2H, m), 6.53 (1H, d), 6.87 (1H, d), 7.03 (1H, d), 7.13 (1H, d), 7.44-7.52 (1H, m), 7.62 (1H, d), 7.74-7.88 (2H, m), 8.10 (1H, d).

### Example 252

### 6′-(((1S,3S)-3-((3-Oxo-3,4-dihydropyrazin-2-yl)amino)cyclopentyl)amino)-2H-[1,3′-bipyridin]-2-one—compound 252

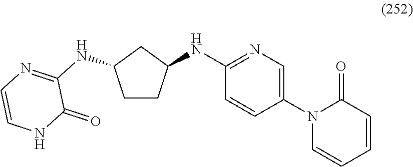 [SMILES: O=c1[nH]ccnc1N[C@H]1CC[C@H](Nc2ccc(-n3ccccc3=O)cn2)C1]

A mixture of 6′-(((1S,3S)-3-((4-(4-methoxybenzyl)-3-oxo-3,4-dihydropyrazin-2-yl)amino)cyclopentyl)amino)-2H-[1,3′-bipyridin]-2-one compound i-78b (190 mg, 0.39 mmol) in DCM (10 mL) was treated with trifluoromethanesulfonic acid (294 mg, 1.96 mmol) at rt and it was stirred for 5 h. The reaction mixture was concentrated under reduced pressure and the obtained material purified by preparative HPLC (PrepMethod R, gradient: 0-15%) to give the title compound (107 mg, 73%) as a white solid. HRMS (ESI) m/z [M+H]⁺ calcd for C₁₉H₂₁N₆O₂: 365.1720, found: 365.1702. ¹H NMR (300 MHz, DMSO-d₆) δ ppm 1.45-1.71 (2H, m), 1.84-2.21 (4H, m), 4.25-4.38 (2H, m), 6.28 (1H, dt), 6.45 (1H, d), 6.55-6.59 (2H, m), 6.71 (1H, d), 7.10 (2H, brs), 7.44-7.51 (2H, m), 7.60 (1H, dd), 7.95 (1H, d), 11.64 (1H, brs).

### Example 253

### 6′-(((1S,3S)-3-((5-Methyl-3-oxo-3,4-dihydropyrazin-2-yl)amino)cyclopentyl)amino)-2H-[1,3′-bipyridin]-2-one—compound 253

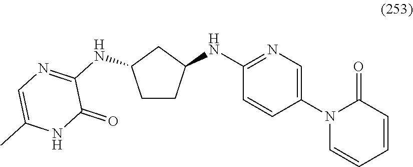 [SMILES: Cc1cnc(N[C@H]2CC[C@H](Nc3ccc(-n4ccccc4=O)cn3)C2)c(=O)[nH]1]

A mixture of 6′-(((1S,3S)-3-((4-(4-methoxybenzyl)-5-methyl-3-oxo-3,4-dihydropyrazin-2-yl)amino)cyclopentyl)amino)-2H-[1,3′-bipyridin]-2-one compound i-79b (80 mg, 0.16 mmol) in DCM (5 mL) was treated with trifluoromethanesulfonic acid (72 mg, 0.48 mmol) at rt and it was stirred for 3 h. The reaction mixture was concentrated under reduced pressure and the obtained material purified by preparative HPLC (PrepMethod 0, gradient: 10-25%) to give the title compound (30 mg, 49%) as a white solid. HRMS (ESI) m/z [M+H]⁺ calcd for C₂₀H₂₃N₆O₂: 379.1876, found: 379.1914. ¹H NMR (300 MHz, DMSO-d₆) δ ppm 1.40-1.62 (2H, m), 1.80-1.97 (5H, m), 2.06-2.18 (2H, m), 4.24-4.37 (2H, m), 6.26 (1H, dt), 6.44 (1H, d), 6.50-6.53 (2H, m), 6.61 (1H, d), 6.90 (1H, d), 7.39 (1H, dd), 7.47 (1H, ddd), 7.60 (1H, dd), 7.92 (1H, d), 11.58 (1H, brs).

### Example 259

### 6′-(((1S,3S)-3-((5-Methylpyrazin-2-yl)amino)cyclopentyl)amino)-5-((methylsulfonyl)methyl)-2H-[1,3′-bipyridin]-2-one—compound 259

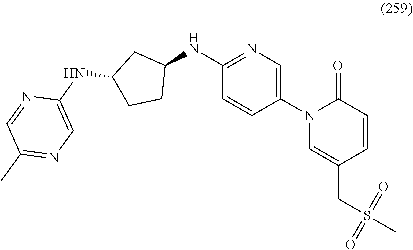

Hydrogen peroxide (30% in water, 150 mg, 1.33 mmol) was added to a mixture of 6′-(((1S,3S)-3-((5-methylpyrazin-2-yl)amino)cyclopentyl)amino)-5-((methylthio)methyl)-2H-[1,3′-bipyridin]-2-one compound i-103d (56 mg, 0.13 mmol) and sodium tungstate (3.9 mg, 0.01 mmol) in THE (5 mL) at 0° C. The mixture was warmed to 25° C. and stirred for 16 h. The reaction mixture was poured into water (100 mL) and the aqueous layer was extracted with EtOAc (2×100 mL). The combined organic layers were dried over Na₂SO₄, filtered and evaporated and the obtained material purified by preparative HPLC (PrepMethod D, gradient: 10-31%) to give the title compound (1.6 mg, 3%) as a white solid. HRMS (ESI) m/z [M+H]⁺ calcd for C₂₂H₂₇N₆O₃S: 455.1860, found: 455.1888. ¹H NMR (300 MHz, DMSO-d₆) δ ppm 1.44-1.53 (2H, m), 1.87 (2H, t), 2.09-2.19 (2H, m), 2.23 (3H, s), 2.94 (3H, s), 4.25-4.36 (4H, m), 6.49 (1H, d), 6.54 (1H, d), 6.86 (1H, d), 6.98 (1H, d), 7.43 (1H, dd), 7.49 (1H, dd), 7.71 (1H, d), 7.81-7.82 (2H, m), 7.96 (1H, d).

### Example 274

### 1-(6-(((1S,3S)-3-((5-Methylpyrazin-2-yl)amino)cyclopentyl)amino)pyridin-3-yl)-3,4-dihydroquinolin-2 (1H)-one—compound 274

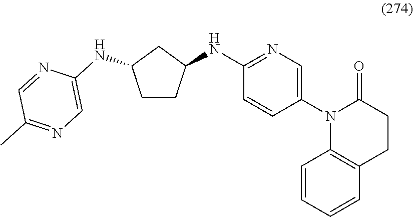 [SMILES: Cc1cnc(N[C@H]2CC[C@H](Nc3ccc(N4C(=O)CCc5ccccc54)cn3)C2)cn1]

In a slight variation of GM3 (1S,3S)—N-(5-iodopyridin-2-yl)-N3-(5-methylpyrazin-2-yl)cyclo-pentane-1,3-diamine compound i-32a (100 mg, 0.25 mmol) was reacted with 3,4-dihydro-2 (1H)-quinolinone (CAS Reg. No. 553-03-7) (37 mg, 0.25 mmol), K₂CO₃ (70 mg, 0.51 mmol), rel-(1R,2R)—N¹,N²-dimethylcyclohexane-1,2-diamine (8 μL, 0.05 mmol) and Cu(I)I (9.6 mg, 0.05 mmol) in 1,4-dioxane (1.5 mL) at 100° C. for 18 h under nitrogen. The reaction mixture was concentrated under reduced pressure and the obtained residue taken up with DMSO, filtered and purified by preparative HPLC (PrepMethod SFC-E) to afford the title compound (28 mg, 27%) as a white solid. HRMS (ESI) m/z [M+H]⁺ calcd for C₂₄H₂₇N₆O: 415.2240, found: 415.2242. ¹H NMR (600 MHz, DMSO-d₆) δ 1.4-1.58 (2H, m), 1.88 (2H, hept), 2.08-2.18 (2H, m), 2.23 (3H, s), 2.66-2.68 (2H, m), 2.98 (2H, dd), 4.23 (1H, m), 4.31 (1H, q), 6.35 (1H, dd), 6.57 (1H, d), 6.85 (2H, dd), 6.95 (1H, td), 7.07 (1H, td), 7.13-7.29 (2H, m), 7.75 (1H, d), 7.76-7.84 (2H, m).

### Example 276

### 1-(6-(((1S,3S)-3-((5-Methylpyrazin-2-yl)amino)cyclopentyl)amino)pyridin-3-yl)-3,4-dihydro-1,8-naphthyridin-2 (1H)-one—compound 276

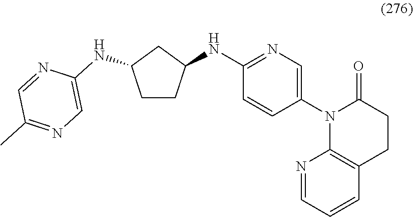 [SMILES: Cc1cnc(N[C@H]2CC[C@H](Nc3ccc(N4C(=O)CCc5cccnc54)cn3)C2)cn1]

In a slight variation of GM3 (1S,3S)—N¹-(5-iodopyridin-2-yl)-N³-(5-methylpyrazin-2-yl)cyclo-pentane-1,3-diamine compound i-32a (100 mg, 0.25 mmol) was reacted with 3,4-dihydro-1,8-naphthyridin-2 (1H)-one (CAS Reg. No. 40000-79-1) (38 mg, 0.25 mmol), K₂CO₃ (70 mg, 0.51 mmol), rel-(1R,2R)—N¹,N²-dimethylcyclohexane-1,2-diamine (10 μL, 0.06 mmol) and Cu(I)I (12 mg, 0.06 mmol) in 1,4-dioxane (1.5 mL) at 100° C. for 17 h under nitrogen. Another amount of Cu(I)I (48 mg, 0.25 mmol) and rel-(1R,2R)—N¹,N²-dimethylcyclohexane-1,2-diamine (40 μL, 0.25 mmol) were added and stirring at 100° C. continued for 21 h under nitrogen. The reaction mixture was concentrated under reduced pressure and the obtained residue taken up with DMSO, filtered and purified by preparative HPLC (PrepMethod SFC-A) to afford the title compound (10 mg, 10%) as a white solid. MS (ESI): m/z [M+H]⁺ 416.2. HRMS (ESI) m/z [M+H]⁺ calcd for C₂₃H₂₆N₇O: 416.2194, found: 416.2196. ¹H NMR (600 MHz, DMSO-d₆) δ 1.44-1.56 (2H, m), 1.90 (2H, dq), 2.16 (2H, td), 2.24 (3H, s), 2.75 (2H, dd), 2.97-3.04 (2H, m), 4.2-4.35 (2H, m), 6.60 and 7.26 (1H in total, s), 6.86 (1H, d), 7-7.03 (2H, m), 7.67 (1H, dd), 7.74 (1H, d), 7.8-7.83 (2H, m), 8.03 (1H, dd).

### Example 278

### 2-(6-(((1S,3S)-3-((5-Methylpyrazin-2-yl)amino)cyclopentyl)amino)pyridin-3-yl)-3-oxo-2,3-dihydropyridazine-4-carbonitrile—compound 278

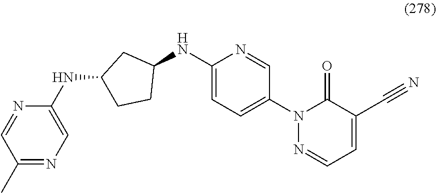 [SMILES: Cc1cnc(N[C@H]2CC[C@H](Nc3ccc(-n4nccc(C#N)c4=O)cn3)C2)cn1]

In a slight variation of GM2 the crude TFA salt of 2-(6-(((1S,3S)-3-aminocyclopentyl)amino)pyri-din-3-yl)-3-oxo-2,3-dihydropyridazine-4-carbonitrile compound i-106b (170 mg, 0.27 mmol) was reacted with 2-bromo-5-methylpyrazine (CAS Reg. No. 98006-90-7) (92 mg, 0.53 mmol), K₂CO₃ (110 mg, 0.80 mmol) and Pd-PEPPSI-IpentCl 2-methylpyridine (22 mg, 0.03 mmol) in 1,4-dioxane (10 mL) at 100° C. for 15 h under nitrogen. The reaction mixture was filtered through a pad of Celite, the filter cake washed with EtOAc (3×20 mL) and the combined filtrates concentrated under reduced pressure. The obtained material was purified by preparative TLC (MeOH:DCM=1:10) followed by preparative HPLC (PrepMethod E, gradient: 5-27%) to give the title compound as an orange solid (24 mg, 23%). HRMS (ESI) m/z [M+H]⁺ calcd for C₂₀H₂₁N₈O: 389.1832, found: 389.1830. ¹H NMR (400 MHz, DMSO-d₆) δ ppm 1.43-1.54 (2H, m), 1.87 (2H, t), 2.10-2.19 (2H, m), 2.23 (3H, s), 4.25 (1H, q), 4.34 (1H, q), 6.53 (1H, d), 6.87 (1H, d), 7.09 (1H, d), 7.54 (1H, dd), 7.80-7.82 (2H, m), 8.13 (1H, d), 8.20 (1H, d), 8.22 (1H, d).

### Example 282

### 4-(Difluoromethoxy)-2-(6-(((1S,3S)-3-((5-methylpyrazin-2-yl)amino)cyclopentyl)amino)pyridin-3-yl)pyridazin-3 (2H)-one—compound 282

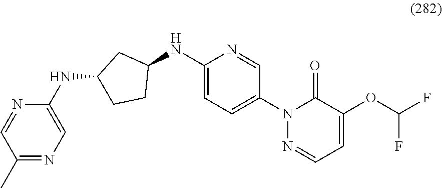 [SMILES: Cc1cnc(N[C@H]2CC[C@H](Nc3ccc(-n4nccc(OC(F)F)c4=O)cn3)C2)cn1]

In a slight variation of GM3 (1S,3S)—N¹-(5-iodopyridin-2-yl)-N³-(5-methylpyrazin-2-yl)cyclopentane-1,3-diamine compound i-32a (150 mg, 0.38 mmol), 4-(difluoromethoxy)pyrida-zin-3 (2H)-one compound i-107e (185 mg, 1.14 mmol), K₂CO₃ (157 mg, 1.14 mmol), Cu(I)I (72 mg, 0.38 mmol) and rel-(1R,2R)—N¹,N²-dimethylcyclohexane-1,2-diamine (54 mg, 0.38 mmol) were reacted in DMF (10 mL) at 100° C. for 18 h. The reaction mixture was diluted with EtOAc (150 mL) and the organic layer was washed sequentially with water (3×75 mL) and sat. brine (3×50 mL), dried over Na₂SO₄, filtered and evaporated and the crude material was purified by preparative TLC (7 M NH₃ in MeOH:DCM=1:15) followed by preparative HPLC (PrepMethod D, gradient: 20-38%) to give the title compound (68 mg, 42%) as a yellow solid. HRMS (ESI) m/z [M+H]⁺ calcd for C₂₀H₂₂F₂N₇O₂: 430.1798, found: 430.1808. ¹H NMR (300 MHz, DMSO-d₆). δ ppm 1.41-1.55 (2H, m), 1.87 (2H, t), 2.09-2.20 (2H, m), 2.24 (3H, s), 4.20-4.39 (2H, m), 6.53 (1H, d), 6.85 (1H, d), 7.00 (1H, d), 7.24 (1H, d), 7.46 (1H, t), 7.52 (1H, dd), 7.81-7.82 (2H, m), 8.02 (1H, d), 8.11 (1H, d). ¹⁹F NMR (282 MHz, DMSO-d₆) b ppm −84.50 (2F, s).

### Assays and Methods Used

### Assay 1—Biochemical Human PCSK9 Assay

This assay measures binding of compounds to PCSK9 by homogenous time-resolved fluorescence resonance energy transfer (TR-FRET).

To determine the IC₅₀ of inhibitors of the interaction between the human PCSK9 and Alexa647 labelled small molecule, fluorescent probe displacement was monitored by homogenous TR-FRET technology. Upon binding of a terbium (Tb) cryptate conjugated anti-His mouse antibody (mAb Anti-6His Tb cryptate Gold, Cisbio) to PCSK9-TEV-His6, the displacement of the probe from PCSK9 was assessed by reduction of the proximity and FRET signal between the Tb cryptate that serves as a FRET-donor and the Alexa647 probe that serves as acceptor.

Recombinantly expressed and purified PCSK9-TEV-His6 (1 nM) was mixed with a fluorescent probe (5 nM) and anti His-Tb-cryptate antibody (0.2 nM) in assay buffer (10 mM HEPES/NaOH, pH 7.4, 150 mM NaCl, 0.005 (v/v) % Tween 20). 6 μL were subsequently added to an assay-ready plate containing 0.06 μL of controls and test compound 10 dose-response serial dilutions starting at a concentration of 10 mM (with 100 μM top and 3.2 nM lowest final concentration) by using Certus flex dispenser. The plate was sealed, and the reaction was incubated overnight (18-24h) at RT in the dark.

FRET signal quantification was achieved by PHERAstar FSX (BMG) plate reader. The created data file contained the emission of FRET acceptor channel (665 nm, probe), FRET donor channel (620 nm, Tb-cryptate, excitation at 337 nm) and the FRET ratio (665 nm/620 nm signal×10.000) which was used for calculation of a test compound's IC₅₀.

### Assay 2—Biophysical Human PCSK9 Assay

This assay measures binding of compounds to PCSK9 by SPR (“surface plasmon resonance”, a biophysical method).

The SPR binding experiments were performed on a Biacore S200 optical biosensor unit at 30° C. A Series S Sensor Chip SA that is designed to bind biotinylated molecules for interaction analysis in Biacore systems was equilibrated at room temperature prior to use. The running buffer for protein tethering and subsequent ligand binding experiments was 10 mM HEPES pH 7.4, 150 mM NaCl, 0.05% (v/v) Tween 20 pH 7.4.

For the surface tethering of PCSK9, biotinylated human PCSK9 (31-692)-Avi-His6 at a concentration of 0.5 mg/mL was used. Prior to the surface tethering, the surface was exposed to a solution of 50 mM NaOH, 500 mM NaCl via 3 consecutive injections of this solution with a contact time of 60 s and a flowrate of 10 μL min⁻¹ to remove non-conjugated streptavidin. The PCSK9 protein was diluted to a concentration of 20 μg/mL using running buffer and injected with a contact time of 180-300 s and a flowrate of 10 μL min⁻¹ over a single flow channel (typically flow channel 2 or flow channel 4) with the aim to achieve protein capture levels of >5000 response units (RU). Remaining biotin binding sites were blocked via 2 consecutive injections of a 10 μM D-biotin solution in running buffer with a contact time of 60 s and a flowrate of 10 μL min⁻¹ over all flow-channels. Flow-channels 1 and 3 typically served as a reference surface throughout the subsequent binding experiments.

The binding experiments were all performed at a flow rate of 30 PL min⁻¹ and by employing the method of single-cycle kinetics. This approach involves the sequential injection of a compound concentration series without regeneration steps. A contact time between 90-150 s was selected, which was followed by a 40 min dissociation phase to allow for a proper estimation of the dissociation rate constant. Test compounds were delivered in DMSO at a concentration of 10 mM and a digital dispenser HP D300 was used to set up the compound concentration series using 6 concentrations. The tested concentrations have been 30, 100, 300, 1000, 3000 and 10000 nM. Prior to injecting any compound, the surfaces were equilibrated by injecting running buffer over them in three separate pulses. The data collection rate was set to 10 Hz.

The raw sensorgrams of the compound injections were first subjected to reference subtraction (subtracting the signal from flow channel 1 and/or 3 from the signal from channels 2 and/or 4 respectively) and then blank subtraction (subtracting the signal from injecting DMSO controls from the reference subtracted data). The resulting double-referenced sensorgrams were then fitted using a 1:1 binding interaction model using the manufactures software package to extract kinetic- and affinity data. Active compounds have been defined by creating a detectable binding signal at the highest compound concentration (10 mM) of 3 RUs. Kinetic- and affinity data on active compounds are only provided for those compounds where the binding signal at the highest compound concentration (10 mM) is 50% of the theoretical maximum binding signal for a 1:1 binding interaction (Rₘₐₓ, typically between 15-20 RUs) in order to enable a proper fitting of the data.

### Assay 3—PCSK9 LDL-C Uptake Assay

This assay measures for PCSK9 antagonist activity based on a test compound's capacity to restore LDL-C-uptake in HepG2 cells. The assay is based on exogenous PCSK9 and LDL-C complexed with a pH-sensitive dye. Outside the cells, at neutral pH, the pHrodo Red-LDL is dimly fluorescent but upon LDLR mediated endocytosis it fluoresces brightly. PCSK9 traffics the LDLR to intracellular degradation and reduces uptake of LDL-C. Inhibition of PCSK9 reduces LDLR degradation and the increased LDL-C uptake is quantified by fluorescence microscopy.

### Preparation of Assay Reagents

Cell medium: MEM supplemented with 10% FBS, 1×NEAA and Sodium Pyruvate Assay medium: OptiMem supplemented with penicillin/streptomycin

### Cells: HepG2 (ATCC #HB-8065)

PCSK9: Sequence:
MGTVSSRRSW WPLPLLLLLL LLLGPAGARA QEDEDGDYEE
LVLALRSEED GLAEAPEHGT TATFHRCAKD PWRLPGTYVV
VLKEETHLSQ SERTARRLQA QAARRGYLTK ILHVFHGLLP
GFLVKMSGDL LELALKLPHV DYIEEDSSVF AQSIPWNLER
ITPPRYRADE YQPPDGGSLV EVYLLDTSIQ SDHREIEGRV
MVTDFENVPE EDGTRFHRQA SKCDSHGTHL AGVVSGRDAG
VAKGASMRSL RVLNCQGKGT VSGTLIGLEF IRKSQLVQPV
GPLVVLLPLA GGYSRVLNAA CQRLARAGVV LVTAAGNFRD
DACLYSPASA PEVITVGATN AQDQPVTLGT LGTNFGRCVD
LFAPGEDIIG ASSDCSTCFV SQSGTSQAAA HVAGIAAMML
SAEPELTLAE LRQRLIHFSA KDVINEAWFP EDQRVLTPNL
VAALPPSTHG AGWQLFCRTV WSAHSGPTRM ATAIARCAPD
EELLSCSSFS RSGKRRGERM EAQGGKLVCR AHNAFGGEGV
YAIARCCLLP QANCSVHTAP PAEASMGTRV HCHQQGHVLT
GCSSHWEVED LGTHKPPVLR PRGQPNQCVG HREASIHASC
CHAPGLECKV KEHGIPAPQE QVTVACEEGW TLTGCSALPG
TSHVLGAYAV DNTCVVRSRD VSTTGSTSEE AVTAVAICCR
SRHLAQASQE LQENLYFQGH HHHHH

### Step by Step Protocol for Running the Assay:

### Day 1

1. Cryopreserved HepG2 cells were thawed in assay medium and centrifuged for 5 min at 250 g. Supernatant was discarded and the cell pellet resuspended in cell medium and counted with a Nucleocounter. Cells were again centrifuged 5 min at 250 g and the pellet was resuspended to 500000 cells/mL in assay medium.
2. 20 μL of above cell mix was dispensed into black pclear TC treated 384 well plates with Multidrop Combi and left at room temperature for 20 minutes.
3. Plates were incubated at 37° C., 5% CO₂ for 24 h

### Day 2

1. Test compounds were prepared in concentration response in DMSO with a half-log dilution factor in DMSO in Echo 384 LDV plates starting at 10 mM.
2. 30 nL of above test compounds were dispensed with Echo 655 to cells for a top concentration of 10 μM
3. 10 μL PCSK9 in assay medium was dispensed to cells with Multidrop Combi for 125 nM final concentration in the wells.
4. Plates were incubated at 37° C., 5% CO₂ for 24 h

### Day 3

1. 10 μL pHrodo Red-LDL in assay medium was dispensed to cells with Multidrop Combi for 6 μg/mL
2. Plates were incubated at 37° C., 5% CO₂ and imaged with the Incucyte S3 after 4 h and 24 h

Image data was processed using Incucyte 2021, a software to identify cells and red fluorescent intensity. Screener was used to further process data. Data was normalized as % effect of the signal between median values of the on-plate controls of DMSO and 100 nM Evolocumab according to the formula % effect=−100*(x−DMSO)/(Evolocumab−DMSO) where x equals the measured signal. Concentration response data of the normalized values were fitted using a four-parameter logistic fit.

### Assay 4—In Vivo Assessment of Compound Effects on Plasma LDL-C Levels

A liver-specific human PCSK9 knock-in mouse model (hPCSK9-KI) was generated by expressing human PCSK9 under the control of a mouse albumin promoter/enhancer in C57BL/6N mice as previously described in Carreras 2019. For all in vivo studies, heterozygous male hPCSK9-KI mice and their wildtype littermates were housed individually, and heterozygous female hPCSK9-KI mice were house in groups of 4-6 mice per cage. All animals had free access to enrichment and were housed in a temperature-controlled room (22° C.) with a 12:12-h light/dark cycle. They were fed a chow diet and water ad libitum.

For in vivo assessment of test compound activity, mice were randomised into experimental groups of for example 8 mice per group. Mice were ˜12 weeks of age at the time of study start. On day −1, blood samples were drawn from the tail vein and baseline levels of plasma LDL-C were assessed using an enzymatic method. Baseline plasma levels of human PCSK9 were assessed by ELISA. Starting on day 0, test compounds were dosed by oral gavage at for example 15 mg/kg BID for up to 21 days. Blood samples were drawn from the tail vein and plasma levels of LDL-C and human PCSK9 were assessed using the afore mentioned methods.

Assay 5—hERG Assay (Human Ether-á-go-go-Related Gene)

This assay (human Ether-á-go-go-Related Gene) measures activity of the compounds at the potassium ion channel hERG (human Ether-á-go-go-Related Gene).

Experiments were performed on the SyncroPatch 384PE high throughput patch clamp platform at room temperature and medium resistance chips with 4 patch holes per site. Chinese hamster ovary K1 (CHO) cell lines over-expressing the ion channel of choice (hERG) were used in assay-ready format and kept in liquid nitrogen or were used from live culture. Cells were either thawed and diluted in HBSS or were detached from flasks and resuspended in HBSS. HBSS comprised 140 mM NaCl, 4 mM KCl, 10 mM HEPES and 5 mM Glucose (pH7.4). The internal patch clamp solution was KF 120 mM, KCl 20 mM, HEPES 10 mM, EGTA 10 mM, and 25 PM Escin (pH 7.2). After the sealing process was complete, the external solution was exchanged for external patch clamp solution comprising NaCl 80 mM, KCl 4 mM, HEPES 10 mM, CaCl₂ 2 mM, MgCl₂ 1 mM, glucose 5 mM, and NMDG 60 mM (pH 7.4). All solutions were stored at room temperature, except Escin, which was stored at 4° C. All test compounds were dispensed in greiner-bio 384 well plates and tested in a 6 point cumulative assay (final DMSO concentration 0.33%). Only wells that passed previously agreed acceptance criteria for this platform were used in this analysis (30 MegaOhm seal resistance, Z prime >0.4 and current size >0.2 nA).

High Concentration hERG

Experiments were performed on the QPatchlI high throughput patch clamp platform at room temperature using single holes QChips. Chinese hamster ovary K1 (CHO) cell lines over-expressing the ion channel of choice (hERG) were used from live culture. All solutions were stored at 4° C. or −20° C. All compounds were dispensed as 10 or 50 mM DMSO stocks, in 96 well plates and diluted to a format that allowed testing in a 6 point cumulative assay (final DMSO concentration 2% or 0.4% DMSO). Only wells that passed previously agreed acceptance criteria for this platform were used in this analysis (500 MegaOhm seal resistance and current size >0.2 nA, with positive controls including Verapamil and DMSO being consistent).

### Assay 6—GSK3β Assay (ThermoFisher Assay)

This assay measures the activity of the compounds at GSK3β (Glycogen synthase kinase-3 beta).

The test compounds were screened in 1% DMSO (final) in the well. For 10 point titrations, 3-fold serial dilutions are conducted from the starting concentration of 10 μM.

### Assay Protocol

Bar-coded Corning, low volume NBS, black 384-well plate
1. 2.5 μL—4× Test Compound or 100 nL 100× plus 2.4 μL kinase buffer
2. 5 μL—2× Peptide/Kinase Mixture
3. 2.5 μL—4×ATP Solution
4. 30-second plate shake
5. 60-minute Kinase Reaction incubation at room temperature
6. 5 μL—Development Reagent Solution
7. 30-second plate shake
8. 60-minute Development Reaction incubation at room temperature
9. Read on fluorescence plate reader and analyse the data

In step 2, the 2×GSK3β (GSK3 beta)/Ser/Thr (Glycogen synthase kinase-3 beta/Serine/Threonine) 09 mixture is prepared in 50 mM HEPES (4-(2-hydroxyethyl)-1-piperazineethanesulfonic acid) pH 7.5, 0.01% BRIJ-35, 10 mM MgCl₂, 1 mM EGTA (egtazic acid). The final 10 μL Kinase Reaction consists of 0.22-0.92 ng GSK3β (GSK3 beta) and 2 μM Ser/Thr 09 in 50 mM HEPES pH 7.5, 0.01% BRIJ-35, 10 mM MgCl₂, 1 mM EGTA.

In step 3 the ATP Solution is diluted to a 4× working concentration in Kinase Buffer (50 mM HEPES pH 7.5, 0.01% BRIJ-35, 10 mM MgCl2, 1 mM EGTA).

In Step 6 the Development Reagent is diluted 1:512 in Development Buffer (10× Novel PKC Lipid Mix: 2 mg/mL Phosphatidyl Serine, 0.2 mg/mL DAG in 20 mM HEPES, pH 7.4, 0.3% CHAPS).

Graphing Software

SelectScreen® Kinase Profiling Service uses XLfit from IDBS. The dose response curve is curve fit to model number 205 (sigmoidal dose-response model). If the bottom of the curve does not fit between −20% & 20% inhibition, it is set to 0% inhibition. If the top of the curve does not fit between 70% and 130% inhibition, it is set to 100% inhibition.

TABLE 10
Activity results in Assays 1, 2, 5 and 6 for compounds 1-80

| | | | Assay 5 | Assay 6 |
|---|---|---|---|---|
| | Assay 1 (PCSK9 | Assay 2 (PCSK9 | (hERG Hu CHO | (GSK3β |
| | Hu Bind TRF) | BIAcore DBA | IF EPh CR- | FRET-Z-Lyte CR) |
| | GMean | CR) GMean | Syncro) | GMean IC₅₀ (μM) |
| Example | IC₅₀ (nM) | Kd (nM) | GMean IC₅₀ (μM) | ATP Conc: 10 (μM) |
| 1 | 2747 | 1700 | >40.1 | |
| 2 | 112 | 64 | >40.0 | >10.0 |
| 3 | 40 | 39 | 24.6 | >10.0 |
| 4 | 104 | 81 | >40.0 | >10.0 |
| 5 | 499 | 412 | 18.5 | 0.713 |
| 6 | 1163 | 863 | >40.1 | |
| 7 | 3502 | | >40.0 | >10.0 |
| 8 | 257 | | >40.0 | >10.0 |
| 9 | 677 | | >40.0 | 5.6 |
| 11 | 3.2 | | 20.2 | >10.0 |
| 12 | 13 | | >40.0 | >10.0 |
| 13 | 4.0 | | 35.6 | >10.0 |
| 14 | 52 | | >40.0 | >10.0 |
| 15 | 118 | | >40.0 | 0.874 |
| 16 | 103 | | >40.0 | >10.0 |
| 17 | 53 | | >40.0 | >10.0 |
| 18 | 17 | 14 | >40.0 | >10.0 |
| 19 | 5.3 | | 24.6 | >10.0 |
| 20 | 17 | | >40.0 | >10.0 |
| 21 | 11 | | 33.2 | >10.0 |
| 22 | 107 | 93 | >40.0 | >10.0 |
| 23 | 8.8 | | 35.9 | >10.0 |
| 24 | 4.3 | 4.0 | 20.5 | >10.0 |
| 25 | 28 | | >40.0 | >10.0 |
| 26 | 24.3 | | 14.3 | >10.0 |
| 27 | 1.7 | 3.1 | 6.35 | >10.0 |
| 28 | 43 | | 25.0 | 0.160 |
| 29 | 14 | | 11.1 | >10.0 |
| 30 | 27 | | 32.2 | >10.0 |
| 31 | 3.7 | | 8.2 | >10.0 |
| 32 | 7.9 | 20 | 3.5 | >10.0 |
| 33 | 12 | 13 | >40.0 | >10.0 |
| 34 | 5.1 | | 15.4 | >10.0 |
| 35 | 2.0 | 3.0 | 4.7 | >10.0 |
| 36 | 4.1 | 5.3 | >40.0 | >10.0 |
| 37 | 6.5 | 5.5 | >40.0 | >10.0 |
| 38 | 2.2 | | 25.1 | >10.0 |
| 39 | 34 | 33 | >40.0 | >10.0 |
| 40 | 67 | | 35.5 | 0.192 |
| 41 | 57 | | >40.0 | >10.0 |
| 42 | 5.8 | 5.0 | >40.0 | >10.0 |
| 43 | 22 | | >40.0 | >10.0 |
| 44 | 5.5 | 4.5 | >40.0 | >10.0 |
| 45 | 1.5 | 1.5 | 23.1 | >10.0 |
| 46 | 3.0 | 4.7 | 33.0 | >10.0 |
| 47 | 2.1 | 1.9 | >40.0 | >10.0 |
| 48 | 113 | | 18.4 | >10.0 |
| 49 | 58 | | >40.0 | >10.0 |
| 77 | 1533 | | >40.0 | >10.0 |
| 78 | 1633 | | >40.0 | >10.0 |
| 79 | 827 | 422 | >40.0 | >10.0 |
| 80 | 1033 | | >40.0 | >10.0 |

TABLE 11
Activity results in Assays 1, 2, 5 and 6 for compounds 81-148

| | | Assay 2 (PCSK9 | Assay 5 | Assay 6 |
|---|---|---|---|---|
| | Assay 1 (PCSK9 | BIAcore DBA | (hERG Hu CHO | (GSK3β |
| | Hu Bind TRF) | CR) | IF EPh CR- | FRET-Z-Lyte CR) |
| | GMean | GMean | Syncro) | GMean IC₅₀ (μM) |
| Example | IC₅₀ (nM) | Kd (nM) | GMean IC₅₀ (μM) | ATP Conc: 10 (μM) |
| 81 | 202 | 66 | >40.0 | 0.498 |
| 82 | 2.5 | 4.1 | 5.5 | >10.0 |
| 83 | 44 | | 5.0 | >10.0 |
| 84 | 11 | | 6.9 | >10.0 |
| 85 | 4.3 | | 5.6 | >10.0 |
| 86 | 73 | 64 | 13.4 | >10.0 |
| 87 | 9.2 | | 18.1 | >10.0 |
| 88 | 4.5 | | 4.5 | >10.0 |
| 89 | 121 | | 6.8 | >10.0 |
| 90 | 15 | | 11.7 | >10.0 |
| 91 | 436 | | 9.6 | >10.0 |
| 92 | 59 | 52 | 4.4 | >10.0 |
| 93 | 87 | | >40.0 | >10.0 |
| 94 | 7.7 | 12 | 17.5 | >10.0 |
| 95 | 114 | | 11.1 | 0.115 |
| 96 | 7.3 | | 12.2 | >10.0 |
| 97 | 63 | 26 | 35.4 | >10.0 |
| 98 | 4.2 | | 10.8 | >10.0 |
| 99 | 40 | | 19.9 | >10.0 |
| 100 | 28 | 45 | >40.1 | >10.0 |
| 101 | 5.5 | | 19.4 | >10.0 |
| 102 | 85 | 122 | 17.7 | >10.0 |
| 103 | 5.7 | 4.7 | >40.1 | >10.0 |
| 104 | 5.8 | | 24.2 | >10.0 |
| 105 | 6.4 | | 20.4 | >10.0 |
| 106 | 4.5 | 3.0 | >40.0 | >10.0 |
| 107 | 105 | | | 0.160 |
| 108 | 3.4 | | 12.7 | >10.0 |
| 109 | 41 | | 19.9 | >10.0 |
| 110 | 48 | | 25.3 | >10.0 |
| 111 | 149 | 128 | 20.8 | >10.0 |
| 139 | 235 | 460 | 4.6 | 0.292 |
| 140 | 309 | 81 | >40.0 | |
| 141 | 592 | 405 | >40.0 | >10.0 |
| 142 | 1606 | 2420 | >35.4 | 0.764 |
| 143 | 2394 | 1540 | >40.0 | >10.0 |
| 144 | 484 | 336 | >35.0 | 2.34 |
| 145 | 960 | 637 | 18.5 | >10.0 |
| 146 | 1481 | 1500 | >40.0 | >10.0 |
| 147 | 5.2 | | 16.6 | >10.0 |
| 148 | 8.0 | | >40.1 | >10.0 |

TABLE 12
Activity results in Assay 3

| | Assay 3 LDL uptake |
|---|---|
| Example | potency (μM) |
| 11 | 0.3 |
| 12 | 0.1 |
| 13 | 0.2 |
| 14 | 0.2 |
| 19 | 0.1 |
| 20 | 0.1 |
| 21 | 0.1 |
| 23 | 0.7 |
| 24 | 0.4 |
| 27 | 0.1 |
| 31 | 0.2 |
| 32 | 0.2 |
| 33 | 0.1 |
| 34 | 0.1 |
| 35 | 0.1 |
| 36 | 0.1 |
| 37 | 0.2 |
| 38 | 0.1 |
| 42 | 0.2 |
| 43 | 0.7 |
| 44 | 0.3 |
| 45 | 0.2 |
| 46 | 0.7 |
| 47 | 0.1 |
| 82 | 0.2 |
| 84 | 0.7 |
| 85 | 0.2 |
| 87 | 0.1 |
| 88 | 0.2 |
| 94 | 0.3 |
| 96 | 0.7 |
| 98 | 0.8 |
| 101 | 0.6 |
| 103 | 0.2 |
| 104 | 0.5 |
| 105 | 0.8 |
| 106 | 0.2 |
| 108 | 0.4 |
| 147 | 0.1 |
| 148 | 0.2 |

TABLE 13
Activity results in Assays 1, 2, 3, 5 and 6 for compounds 149-282

| | | | | Assay 6 | |
|---|---|---|---|---|---|
| | | | Assay 5 | (GSK3β | |
| | | Assay 2 | (hERG Hu | FRET-Z-Lyte | |
| | Assay 1 | (PCSK9 | CHO | CR) | |
| | (PCSK9 Hu | BIAcore | IF EPh CR- | GMean IC₅₀ | Assay 3 LDL |
| | Bind TRF) | DBA CR) | Syncro) | (μM) | uptake |
| Example | GMean | GMean | GMean IC₅₀ | ATP Conc: 10 | potency |
| No | IC₅₀ (nM | Kd (nM) | (μM) | (μM) | (μM) |
| 149 | 3958 | | >40.1 | >10.0 | |
| 150 | 8698 | | >40.1 | >10.0 | |
| 162 | 42 | | >40.1 | >10.0 | |
| 163 | 140 | | 30.7 | >10.0 | |
| 164 | 451 | | >40.1 | >10.0 | |
| 170 | 11 | | >40.1 | 7.0 | 0.2 |
| 171 | 602 | | >40.1 | 2.1 | |
| 172 | 5.3 | | >40.1 | 3.7 | 0.1 |
| 176 | 12 | | >40.1 | >10.0 | 0.1 |
| 177 | 5.4 | | >40.1 | >10.0 | 0.2 |
| 178 | 10 | | >40.1 | >10.0 | 0.1 |
| 179 | 4.7 | | | 6.9 | 0.1 |
| 203 | 7.6 | | >40.1 | >10.0 | 0.3 |
| 204 | 3.5 | | >40.1 | >10.0 | 0.4 |
| 205 | 7.0 | | >40.1 | >10.0 | |
| 206 | 22 | | >40.1 | >10.0 | 0.6 |
| 207 | 8.6 | | >40.1 | >10.0 | 0.4 |
| 209 | 6.3 | | >40.1 | >10.0 | 0.2 |
| 215 | 1.0 | | 16.0 | >10.0 | 0.1 |
| 216 | 2.8 | | 7.3 | >10.0 | 0.3 |
| 218 | 5.4 | | 22.5 | >10.0 | 0.3 |
| 226 | 4.0 | 5.6 | >40.1 | >10.0 | 0.2 |
| 227 | 12 | | >40.1 | >10.0 | |
| 231 | 46 | 35 | >40.1 | >10.0 | |
| 232 | 3.7 | | 39.9 | >10.0 | 0.5 |
| 233 | 24 | | >40.1 | >10.0 | |
| 252 | 2584 | | >40.1 | >10.0 | |
| 253 | 307 | | >40.1 | >10.0 | |
| 259 | 43 | | >40.1 | >10.0 | >0.6 |
| 274 | 8.1 | | 17.1 | >10.0 | |
| 276 | 80 | | >40.0 | >10.0 | |
| 278 | 13 | 12 | >40.1 | >10.0 | |
| 282 | 10 | | >40.1 | >10.0 | |

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the disclosure in diverse forms thereof.

While the disclosure has been described in conjunction with the exemplary embodiments described above, many equivalent modifications and variations will be apparent to those skilled in the art when given this disclosure. Accordingly, the exemplary embodiments set forth above are considered to be illustrative and not limiting. Various changes to the described embodiments may be made without departing from the spirit and scope of the disclosure.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### REFERENCES

A number of publications are cited above in order to more fully describe and disclose the compound of formula (I) and the state of the art to which it pertains. Full citations for these references are provided below. The entirety of each of these references is incorporated herein.

| | Full Reference | DOI |
|---|---|---|
| Elbitar 2016 | Elbitar et al., Expert Opin | 10.1080/13543776.2016.1206080 |
| | Therapeutic Patents 2016 26: 1377- | |
| | 1392. | |
| Abifadel 2003 | Abifadel, et al. Nat. Genet. 2003 | 10.1038/ng1161 |
| | 34: 154-6. | |
| Abifadel 2010 | Abifadel, et al. Expert Opin. Ther. | 10.1517/13543776.2010.518615 |
| | Pat. 2010 20: 1547-71. | |
| Maxwell 2003 | Maxwell, et al. J. Lipid Res. 2003 | 10.1194/jlr.M300203-JLR200 |
| | 44: 2109-19. | |
| Persson 2009 | Persson et al. Endocrinology 2009 | 10.1210/en.2008-1281 |
| | 150: 1140-6. | |
| Langhi 2008 | Langhi et al. FEBS Lett. 2008 | 10.1016/j.febslet.2008.02.038 |
| | 582: 949-55. | |
| Robinson | Robinson et al., Artherosclerosis | 10.1016/j.atherosclerosis.2015.10.023 |
| 2015 | 2015 243: 593-597. | |
| Duan 2012 | W. Duan et al., ChemMedChem | 10.1002/cmdc.201200145 |
| | 2012, 7, 1276-1285. | |
| Carreras | Carreras, A., et al. (2019). In vivo | 10.1186/s12915-018-0624-2 |
| 2019 | genome and base editing of a | |
| | human PCSK9 knock-in | |
| | hypercholesterolemic mouse model. | |
| | BMC biology 17, 4. | |
| Wuts 2014 | P. G. M. Wuts, Greene's Protective | ISBN: 978-1118057483 |
| | Groups in Organic Synthesis 5th ed., | |
| | J. Wiley & Sons, 2014 | |
| Chillemi 1963 | F. Chillemi, G. Palamidassi, | |
| | Farmaco, “Pyrazine derivatives. VI | |
| | Edizione Scientifica 1963, 18(8), | |
| | 557-65 | |

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

### Numbered Statements:

1. A compound of Formula (I)

A-B—C (I)

or a pharmaceutically acceptable salt, tautomeric forms or stereoisomers thereof,

wherein A is of the following:

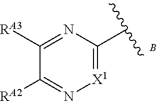

wherein the wavy line indicates the point of attachment to B;

X¹ is C—R^{A1};

R^{A1} is selected from the group consisting of:
(i) H;
(ii) halo;
(iii) CN;
(iv) C₁₋₆ hydrocarbon, optionally substituted by one or more OH, CN, C₁₋₆ acyl, C₁₋₆ alkoxy or one or more halo groups;
(v) C₁₋₆ alkoxy, optionally substituted by OH, one or more halo groups, C₁₋₆ alkyl amido;
(vi) C₁₋₆ alkylester;
(vii) C₁₋₆ alkyl acyl; and
(viii) OH;

R^{A2} is selected from the group consisting of:
(i) H;
(ii) halo;
(iii) CN;
(iv) C₁₋₆ hydrocarbon, optionally substituted by OH, CN, C₁₋₆ acyl, C₁₋₆alkoxy or one or more halo groups;
(v) C₁₋₆ alkoxy, optionally substituted by OH, alkyl amido, or one or more halo groups;
(vi) C₁₋₆ acyl amido (wherein the acyl is optionally substituted by H or methyl);
(vii) C₁₋₆ thioalkyl optionally substituted by C₁₋₆ alkyl ester;
(viii) C₁₋₆ alkyl ester;
(ix) C₁₋₆ alkyl acyl;
(x) C₄₋₅ heterocyclyl;
(xi) C₅ heteroaryl;
(xii) C₁₋₆ alkyl amido optionally substituted by C₁₋₃ alkyl amido, CN, OH, C₂₋₃ alkynyl, C₄₋₆heterocyclyl or C₁₋₃ alkyl wherein the C₁₋₃ alkyl is optionally substituted with one or more halo or OH groups; and
(xiii) OH;

R^{A3} is selected from the group consisting of:
(i) H;
(ii) halo;
(iii) CN;
(iv) C₁₋₆hydrocarbon optionally substituted by OH, CN, C₁₋₆ thioalkyl, C₁₋₆ alkoxy, C₁₋₆ alkyl acyl, C₁₋₆ acyloxy, carboxy, C₁₋₆ alkyl ester, C₁₋₆ alkylamino, —C(═O)NH₂, C₁₋₆ alkyl amido, C₁₋₆ alkyl acylamido, C₁₋₆ alkyl sulfinyl, C₁₋₆ alkyl sulfonyl or one or more halo groups;
(v) OH;
(vi) C₁₋₆ alkoxy, optionally substituted by OH, NH₂, C₄ heterocyclyl or one or more halo groups;
(vii) C₁₋₆ acyloxy;
(viii) C₄ heterocycyl;
(ix) —NH₂;
(x) C₁₋₆ alkylamino, optionally substituted by CN, OH or C₄heterocyclyl;
(xi) C₁₋₆ dialkylamino, optionally substituted by —NH₂;
(xii) C₁₋₆ acylamido (where acyl substituent is H or Me);
(xiii) carbaimidoyl or methyl-carbaimidoyl;
(xiv) carboxyamino;
(xv) C₁₋₆ thioalkyl, optionally substituted by OH or NH₂;
(xvi) C₁₋₆ alkyl sulfinyl;
(xvi) C₁₋₆ alkyl sulfonyl, optionally substituted by one or more halo groups;
(xvii) C₁₋₆ sulfonimodyl;
(xviii) C₁₋₆ alkyl phosphinyl;
(xix) carboxy;
(xx) C(═O)NH₂
(xxi) C₁₋₆ alkyl ester;
(xxii) C₁₋₆ alkyl acyl, optionally substituted by one or more halo groups; and
(xxiii) C₁₋₆ alkyl amido;

or wherein R^{A3} and R^{A2} together with the carbon atoms to which they are bound form:
(i) an optionally substituted C₅₋₇ heterocycle ring;
(ii) an optionally substituted C₅₋₇ heteroaromatic ring;
(iii) an optionally substituted C₆ carboaromatic ring;
(iv) an optionally substituted C₅₋₇ carbocyclic ring
   wherein the optional substituents are selected from C₁₋₆alkyl, halo, C₁₋₆ alkoxy, NH₂, C₁₋₆alkylamino, OH, and CN;

wherein B is of formula (B-1) or (B-2)
i)

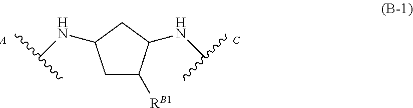

wherein the wavy line indicates the point of attachment to A and C;

R^{B1} is H, OH, ═CHCH₂—OH, —O—C₁₋₄alkyl or C₁₋₄alkyl, wherein the C₁₋₄alkyl is optionally substituted by OH or OMe;
(ii)

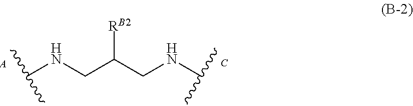

wherein the wavy line indicates the point of attachment to A and C;

R^{B2} is C₁₋₂ alkyl-OH, CH₂CONHMe or C₁₋₃ alkyl;

wherein C is selected from the group consisting of C₆₋₁₀ carboaryl, C₅₋₆ heteroaryl and C₅₋₁₀ heterocyclyl, which groups are optionally substituted by:
(i) C₆₋₁₀ carboaryl, C₄₋₁₀ carbocyclyl, C₅₋₁₀ heteroaryl C₄₋₁₀ heterocyclyl, or C₅₋₁₀ bridged heterocycle, spiro C₆₋₁₂heterocyclyl or a spiro C₆₋₁₂carbocyclyl,

which are themselves optionally substituted by one or more of the following groups:
a) one or two ═O groups;
b) one or more halo groups;
c) CN, NH₂, OH;
d) one or more C₁₋₆ alkyl groups including branched and cyclic and with an optional substituent selected from OH, C₁₋₆ alkyl sulfonyl or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl optionally substituted with methyl;
i) C₄₋₁₀ carbocyclyl with an optional methyl or ═O substituent;
j) C₆₋₁₀ carboaryl with optional substituents of one or more halo groups;
l) P(═O)Me₂;
m) carboxy or CH₂-carboxy;
n) tetrazolyl, CH₂-tetrazolyl, 5-oxo-4H-1,2,4-oxadiazol-3-yl;
(ii) one or more groups selected from carboxy, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido, and di-C₁₋₆ alkyl sulfonamido.

2. The compound of statement 1 or a pharmaceutically acceptable salt thereof, wherein R^{A1} is selected from the group consisting of:
(i) H;
(ii) halo;
(iii) CN;
(iv) C₁₋₆ hydrocarbon, optionally substituted by OH, CN, C₁₋₆ acyl, C₁₋₆ alkoxy or one or more halo groups;
(v) C₁₋₆ alkoxy, optionally substituted by OH, one or more halo groups, C₁₋₆ alkyl amido; and (vi) OH.

3. The compound of statement 1 or 2 or a pharmaceutically acceptable salt thereof, wherein R^{A1} is selected from the group consisting of:
(i) H;
(ii) halo;
(iii) CN;
(iv) C₁₋₆ hydrocarbon, optionally substituted by OH, CN, C₁₋₆ acyl, C₁₋₆ alkoxy or one or more halo groups; and
(v) C₁₋₆ alkoxy, optionally substituted by OH, one or more halo groups, C₁₋₆ alkyl amido.

4. The compound of any one of statements 1-3 or a pharmaceutically acceptable salt thereof, wherein R^{A1} is selected from the group consisting of H, OH, Br, Cl, CN, OCF₂H, OMe, ethoxy, methyl and ethyl.

5. The compound of any of statements 1-4 or a pharmaceutically acceptable salt thereof, wherein R^{A1} is selected from the group consisting of H, Br, Cl, CN, OCF₂H, OMe, ethoxy, methyl and ethyl.

6. The compound of any one of statements 1 to 5 or a pharmaceutically acceptable salt thereof, wherein R^{A1} is selected from the group consisting of H, OH, —OCF₂H, Br and Cl.

7. The compound of any one of statements 1 to 6 or a pharmaceutically acceptable salt thereof, wherein R^{A1} is selected from the group consisting of H, —OCF₂H, Br and Cl.

8. The compound of any one of statements 1 to 7 or a pharmaceutically acceptable salt thereof, wherein R^{A1} is H.

9. The compound of any one of statements 1, 2, 4 or 6 or a pharmaceutically acceptable salt thereof, wherein R^{A1} is OH.

10. The compound of any one of statements 1 to 9 or a pharmaceutically acceptable salt thereof, wherein R^{A} is selected from the group consisting of:
(i) H;
(ii) halo;
(iii) CN;
(iv) C₁₋₆ hydrocarbon, optionally substituted by OH, CN, C₁₋₆acyl, C₁₋₆ alkoxy or one or more halo groups;
(v) OH;
(vi) C₁₋₆ alkoxy, optionally substituted by OH, C₁₋₆ alkyl amido, or one or more halo groups;
(vii) C₁₋₆ alkyl ester;
(viii) C₁₋₆ alkyl acyl;
(ix) C₁₋₆ alkyl amido optionally substituted by C₁₋₃ alkyl amido, CN, C₂₋₃ alkynyl, C₄₋₆ heterocyclyl, or C₁₋₃ alkyl which alkyl is optionally substituted with one or more halo or OH groups; and
(x) C₁₋₆ thioalkyl optionally substituted by C₁₋₆ alkyl ester.

11. The compound of any one of statements 1 to 10 or a pharmaceutically acceptable salt thereof, wherein R^{A} is selected from the group consisting of:
(i) H;
(ii) halo;
(iii) CN;
(iv) C₁₋₆ hydrocarbon, optionally substituted by OH, CN, C₁₋₆ acyl, C₁₋₆ alkoxy or one or more halo groups;
(v) OH;
(vi) C₁₋₆ alkoxy, optionally substituted by OH, C₁₋₆ alkyl amido, or one or more halo groups;
(vii) C₁₋₆ alkyl ester;
(viii) C₁₋₆ alkyl acyl; and
(ix) C₁₋₆ alkyl amido optionally substituted by C₁₋₃ alkyl amido, CN, C₂₋₃ alkynyl, C₄₋₆ heterocyclyl, or C₁₋₃ alkyl which alkyl is optionally substituted with one or more halo or OH groups.

12. The compound of any one of statements 1 to 11 or a pharmaceutically acceptable salt thereof, wherein R^{A} is selected from the group consisting of:
(i) H;
(ii) halo;
(iii) CN;
(iv) C₁₋₆ hydrocarbon, optionally substituted by OH, CN, C₁₋₆acyl, C₁₋₆alkoxy or one or more halo groups;
(v) C₁₋₆ alkoxy, optionally substituted by OH, alkyl amido, or one or more halo groups;
(vi) C₁₋₆ thioalkyl, optionally substituted by C₁₋₆ alkyl ester;
(vii) C₁₋₆ alkyl ester; or
(viii) C₁₋₆ alkyl amido optionally substituted by C₁₋₃ alkyl amido, CN, C₂₋₃ alkynyl, C₄₋₆heterocyclyl or C₁₋₃alkyl wherein the C₁₋₃ alkyl is optionally substituted with one or more halo or OH groups.

13. The compound of any one of statements 1 to 12 or a pharmaceutically acceptable salt thereof, wherein R^{A} is selected from the group consisting of —CN, methyl, Cl, —C(═O)CH₃, —C(═O)OCH₂CH₃, cyclopropyl, —C(═O)NHCH₂C(═O)NH₂, —C(═O)NHCH₂CHCH, —C(═O)NH-oxetane, —C(═O)NHCH₂CHF₂, —C(═O)NHCH₂CH₂OH, —C(═O)NHCH₂CH₃, —C(═O)NH₂, —C(═O)NHCH₃, —C(═O)N(CH₃)₂, —OCF₂H, H, —OMe, and —OCF₃.

14. The compound of any one of statements 1 to 13 or a pharmaceutically acceptable salt thereof, wherein R^{A} is selected from the group consisting of —OCHF₂, methyl, and cyclopropyl.

15. The compound of any one of statements 1 to 12 or a pharmaceutically acceptable salt thereof, wherein R^{A} is selected from the group consisting of CN, methyl, Cl, —C(═O)CH₃, OCHF₂, cyclopropyl, OCF₃, OCH₃, H, —C(═O)NH(CH₃), S—CH₃, —S—CH₂CH₃ or —S—CH₂—C(═O)—O—CH₃

16. The compound of any one of statements 1 to 15 or a pharmaceutically acceptable salt thereof, wherein R^{A3} is selected from the group consisting of:
(i) H;
(ii) halo;
(iii) CN;
(iv) C₁₋₆ hydrocarbon, optionally substituted by OH, CN, C₁₋₆ thioalkyl, C₁₋₆ alkoxy, C₁₋₆ alkyl acyl C₁₋₆ acyloxy, carboxy, C₁₋₆ alkylester, C₁₋₆ alkylamino; —C(═O)NH₂, C₁₋₆ alkyl amido, C₁₋₆ alkylacylamido, C₁₋₆ alkyl sulfinyl, C₁₋₆ alkyl sulfonyl or one or more halo groups;
(v) OH; and
(vi) C₁₋₆ alkoxy, optionally substituted by OH, NH₂, C₄ heterocyclyl or one or more halo groups.

17. The compound of any one of statements 1 to 16 or a pharmaceutically acceptable salt thereof, wherein R^{A1} is selected from the group consisting of H, CF₃, CN, C₁₋₂ alkyl, NH₂ and halo.

18. The compound of any one of statements 1 to 17 or a pharmaceutically acceptable salt thereof, wherein R^{A3} is selected from the group consisting of H, CN and methyl.

19. The compound of any one of statements 1 to 8 or a pharmaceutically acceptable salt thereof, wherein R^{A3} and R^{A2} together with the carbon atoms to which they are bound form an optionally substituted C₆ carboaromatic ring or C₅ heteroaromatic ring, wherein the optional substituents are selected from methyl, NH₂, Cl, F and OMe.

20. The compound of statement 19 or a pharmaceutically acceptable salt thereof, wherein R^{A2} and R^{A3} together with the carbon atoms to which they are bound form:
(i) an optionally substituted C₆ heteroaryl ring; wherein the optional substituent is NH₂
(ii) an optionally substituted C₆carboaryl ring, wherein the optional substituent is F, OMe, Cl; or
(iii) an optionally substituted C₅ heteroaryl or C₅ heterocyclic ring wherein the optional substituents are methyl.

21. The compound of statement 19 or a pharmaceutically acceptable salt thereof, wherein R^{A3} and R^{A2} together with the carbon atoms to which they are bound form: an unsubstituted 2-pyrazole; a 2-pyrrole substituted by methyl; a pyridine optionally substituted by NH₂; or a phenyl optionally substituted by Cl, F or OMe.

22. The compound of any one of statements 1 to 21 or a pharmaceutically acceptable salt thereof, wherein B is of formula (B-1)

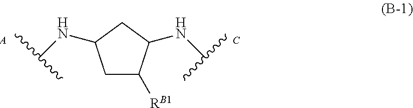

wherein the wavy line indicates the point of attachment to A and C;

wherein R^{B}1 is selected from the group consisting of H, OH, OMe, —O-Ethyl, —CH₂OH, —CH₂CH₂OH and ═CHCH₂—OH.

23. The compound of statement 22 or a pharmaceutically acceptable salt thereof, wherein R^{B1} is selected from the group consisting of —CH₂OH, —CH₂CH₂OH and ═CHCH₂—OH.

24. The compound of any one of statements 1 to 22 or a pharmaceutically acceptable salt thereof, wherein B is of the following formula (B-1a):

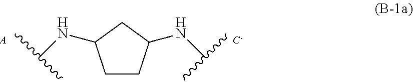

25. The compound of any one of statements 1 to 22 or statement 24 or a pharmaceutically acceptable salt thereof, wherein B is of the following formula (B-1b):

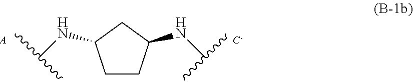

26. The compound of any one of statements 1 to 25 or a pharmaceutically acceptable salt thereof, wherein C is an optionally substituted C₅₋₆ heteroaryl.

27. The compound of any one of statements 1 to 26 or a pharmaceutically acceptable salt thereof, wherein C is an optionally substituted pyridinyl, pyrazinyl or pyrimidinyl, wherein the optional substituents are selected from C₆₋₁₀ carboaryl, C₄₋₁₀ carbocyclyl, C₅₋₁₀ heteroaryl, C₅₋₁₀ heterocyclyl, C₅₋₁₀ bridged heterocyclyl, spiro C₆₋₁₂heterocyclyl or a spiro C₆₋₁₂ carbocyclyl, which are themselves optionally substituted by one or more of the following groups:
a) one or two ═O groups;
b) one or more halo groups;
c) CN, NH₂ or OH;
d) one or more C₁₋₆ alkyl groups including branched and cyclic and with an optional substituent selected from OH, C₁₋₆ alkyl sulfonyl or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl with an optional methyl substituent;
i) C₄₋₁₀ carbocyclyl with an optional methyl or ═O substituent;
j) C₆₋₁₀ carboaryl with optional substituents of one or more halo groups;
l) P(═O)Me₂;
m) carboxy or CH₂-carboxy; and/or
n) tetrazolyl, CH₂-tetrazolyl, 5-oxo-4H-1,2,4-oxadiazol-3-yl.

28 The compound of any one of statements 1 to 27 or a pharmaceutically acceptable salt thereof, wherein C is an optionally substituted pyridinyl, pyrazinyl or pyrimidinyl, wherein the optional substituents are selected from C₆₋₁₀ carboaryl, C₄₋₁₀ carbocyclyl, C₅₋₁₀ heteroaryl, C₅₋₁₀ heterocyclyl, C₅₋₁₀ bridged heterocyclyl, spiro C₆₋₁₂heterocyclyl or a spiro C₆₋₁₂ carbocyclyl, which are themselves optionally substituted by one or more of the following groups:
a) one or two ═O groups;
b) one or more halo groups;
c) CN, NH₂ or OH;
d) one or more C₁₋₆ alkyl groups including branched and cyclic and with an optional substituent selected from OH, or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl;
i) C₄₋₁₀ carbocyclyl with an optional methyl or ═O substituent;
j) C₆₋₁₀ carboaryl with optional substituents of one or more halo groups;
l) P(═O)Me₂;
m) carboxy or CH₂-carboxy; and/or
n) tetrazolyl, CH₂-tetrazolyl, 5-oxo-4H-1,2,4-oxadiazol-3-yl.

29. The compound of any one of statements 1 to 28 or a pharmaceutically acceptable salt thereof, wherein C is substituted by C₆₋₁₀ carboaryl, C₅₋₁₀ heteroaryl, or C₅₋₁₀ heterocyclyl, which are themselves optionally substituted by:
a) one or two ═O groups;
b) one or more halo groups, CN or NH₂;
c) one or more C₁₋₆ alkyl groups, C₁₋₆ alkoxy, or C₁₋₆alkylester, wherein each group is optionally
substituted by one or more halo groups;
d) C₅₋₆ heterocyclyl or C₅₋₆ heteroaryl with an optional methyl substituent;
e) C₆₋₁₀ carboaryl optionally substituted by one or more halo atoms;
f) carboxy or CH₂-carboxy; or
g) tetrazolyl, CH₂-tetrazolyl, 5-oxo-4H-1,2,4-oxadiazol-3-yl.

30. The compound of any one of statements 1 to 27 or a pharmaceutically acceptable salt thereof, wherein C is is an optionally substituted pyridinyl wherein the optional substituents are selected from C₆₋₁₀ carboaryl, C₅₋₁₀ heteroaryl, or C₅₋₁₀ heterocyclyl, which are themselves optionally substituted by:
a) one or two ═O groups;
b) one or more halo groups,
c) CN;
d) one or more C₁₋₆ alkyl optionally substituted by one or more halo groups or C₁₋₆ alkyl sulfonyl;
e) C₁₋₆ alkoxy optionally substituted by one or more halo groups;
f) C₅₋₆ heterocyclyl or C₅₋₆ heteroaryl with an optional methyl substituent;
g) C₆carboaryl optionally substituted by one or more halo atoms; or
h) carboxy.

31. The compound of any one of statements 1 to 25 or a pharmaceutically acceptable salt thereof, wherein C is a C₆₋₁₀ carboaryl, C₅₋₆ heteroaryl and C₅₋₁₀ heterocyclyl, wherein each group is substituted by methyl.

32. The compound of statement 31 or a pharmaceutically acceptable salt thereof, wherein the methyl group on C is at the para position.

33. The compound of any one of statements 1 to 25 or a pharmaceutically acceptable salt thereof, wherein C is of the formula (C-1):

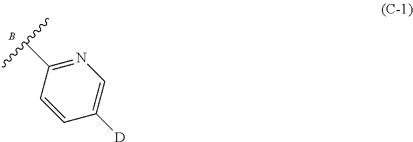

wherein D is C₆₋₁₀ carboaryl, C₅₋₁₀ heteroaryl or C₅₋₁₀ heterocyclyl, each which are themselves optionally substituted by:
i) one or two ═O groups;
ii) one or two C₁₋₄ alkyl groups which can be branched, optionally substituted by —S(═O)₂CH₃;
iii) OMe;
iv) piperazinyl, optionally substituted by methyl;
v) C(═O)OH (carboxy);
vi) Cl;
vii) F;
viii) phenyl, optionally substituted by one or more fluoro;
ix) CN;
x) CF₃;
xi) O—CF₃;
xii) OMe;
xiii) pyrazolyl optionally substituted by methyl, triazolyl or tetrazolyl;
xiv) NH₂;
xv) (CH₃)₂;
xvi) pyridinyl;
xvii) CH₂OH;
xviii) OH; or
xix) P(═O)Me₂.

34. The compound of any one of statements 1 to 25 or a pharmaceutically acceptable salt thereof, wherein C is of the formula (C-1):

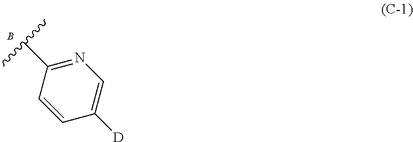

wherein D is C₆₋₁₀ carboaryl, C₅₋₁₀ heteroaryl or C₅₋₁₀ heterocyclyl, each which are themselves optionally substituted by:
i) one or two ═O groups;
ii) one or two C₁₋₄ alkyl groups which can be branched;
iii) OMe;
iv) piperazinyl, optionally substituted by methyl;
v) C(═O)OH (carboxy);
vi) Cl;
vii) F;
viii) phenyl, optionally substituted by one or more fluoro;
ix) CN;
x) CF₃;
xi) O—CF₃;
xii) OMe;
xiii) tetrazolyl, pyrazolyl triazolyl;
xiv) NH₂;
xv) (CH₃)₂;
xvi) pyridinyl;
xvii) CH₂OH;
xviii) OH; or
xix) P(═O)Me₂.

35. The compound of statement 33 or 34 or a pharmaceutically acceptable salt thereof, wherein D is an optionally substituted 6 membered heteroaryl which contains 1 or 2 N atoms, one of which is bonded to (C-1), which is substituted by ═O at the ortho position, wherein the optional substituents are selected from:
i) methyl optionally substituted by —S(═O)₂CH₃;
ii) OMe;
iii) piperazinyl substituted by methyl;
iv) C(═O)OH (carboxy);
v) Cl;
vi) phenyl substituted by fluoro;
vii) CN;
viii) CF₃;
ix) O—CF₃;
x) F; or
xi) pyrazolyl optionally substituted by methyl, triazolyl or tetrazolyl.

36. The compound of statement 33 or 34 or a pharmaceutically acceptable salt thereof, wherein D is an optionally substituted 6 membered heteroaryl which contains 1 or 2 N atoms, one of which is bonded to (C-1), which is substituted by ═O at the ortho position, wherein the optional substituents are selected from:
i) methyl;
ii) OMe;
iii) piperazinyl substituted by methyl;
iv) C(═O)OH (carboxy);
v) Cl;
vi) phenyl substituted by fluoro;
vii) CN;
viii) CF₃;
ix) O—CF₃;
x) F; or
xi) tetrazolyl, pyrazolyl or triazolyl.

37. The compound of statement 33 or 34 or a pharmaceutically acceptable salt thereof, wherein D is an optionally substituted C₁₀ heterocyclyl wherein the optional substituent is an ═O group, optionally wherein D is a 3,4-dihydro-1H-1,8-naphthyridin-2-one (2-oxo-3,4-dihydro-1,8-naphthyridin-1-yl), 3,4-dihydro-1H-quinolin-2-one (2-oxo-3,4-dihydroquinolin-1-yl) 1H-1,8-naphthyridin-2-one (2-oxo-1,8-naphthyridin-1-yl) or 1H-quinolin-2-one (2-oxo-1-quinolyl).

38. The compound of any of statements 33 to 36 or a pharmaceutically acceptable salt thereof, wherein D is of the formula (D-1):

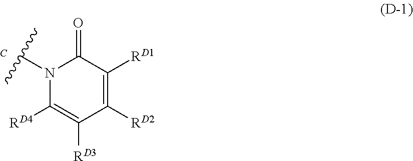

wherein one or two of R^{D1}, R^{D2}, R^{D3} and R^{D4} are selected from:
i) C₁₋₆ alkyl, optionally substituted by C₁₋₆ alkyl sulfonyl or one or more halo groups;
ii) C₁₋₆ alkoxy, optionally substituted by one or more halo groups;
iii) C₅₋₆ heterocyclyl or C₅₋₆ heteroaryl which groups have an optional methyl substituent;
iv) carboxy or CH₂-carboxy;
v) ═O, halo, NH₂ or CN;
vi) phenyl, optionally substituted by one or more halo atoms;

and the rest are H; or

wherein R^{D3} and R^{D4} form an optionally substituted 6 membered carboaromatic, heterocycle or heteroaromatic ring, wherein the optional substituents are selected from OH, methyl, OMe, halo and C(═O)OH;

or wherein R^{D1}, R^{D2}, R^{D3} and R^{D4} are all H.

39. The compound of any of statements 33 to 36 or a pharmaceutically acceptable salt thereof, wherein D is of the formula (D-1):

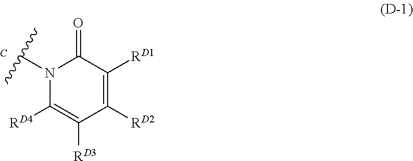

wherein one or two of R^{D1}, R^{D2}, R^{D3} and R^{D4} are selected from:
i) C₁₋₆ alkyl, optionally substituted by one or more halo groups;
ii) C₁₋₆ alkoxy, optionally substituted by one or more halo groups;
iii) C₅₋₆ heterocyclyl or C₅₋₆ heteroaryl with an optional methyl substituent;
iv) carboxy or CH₂-carboxy;
v) ═O, halo, NH₂ or CN;
vi) phenyl, optionally substituted by one or more halo atoms;

and the rest are H; or

wherein R^{D3} and R^{D4} form an optionally substituted 6 membered carboaromatic, heterocycle or heteroaromatic ring, wherein the optional substituents are selected from OH, methyl, OMe, halo and C(═O)OH;

or wherein R^{D1}, R^{D2}, R^{D3} and R^{D4} are all H.

40. The compound of statement 38 or 39 or a pharmaceutically acceptable salt thereof, wherein one or two of R^{D1}, R^{D2}, R^{D3} and R^{D4} are selected from:
i) methyl optionally substituted by CH₂—S(═O)₂CH₃;
ii) OMe;
iii) piperazinyl substituted by methyl;
iv) C(═O)OH (carboxy);
v) Cl;
vi) phenyl substituted by fluoro;
vii) CN;
viii) CF₃;
ix) O—CF₃
x) F;
xi) pyrazolyl optionally substituted by methyl, triazolyl or tetrazolyl;

and the rest of R^{D1}, R^{D2}, R^{D3} and R^{D4} are H.

41. The compound of any one of statements 38-40 or a pharmaceutically acceptable salt thereof, wherein one or two of R^{D1}, R^{D2}, R^{D3} and R^{D4} are selected from:
i) methyl;
ii) OMe;
iii) piperazinyl substituted by methyl;
iv) C(═O)OH (carboxy);
v) Cl;
vi) phenyl substituted by fluoro;
vii) CN;
viii) CF₃;
ix) O—CF₃
x) F;
xi) pyrazolyl, triazolyl or tetrazolyl;

and the rest of R^{D1}, R^{D2}, R^{D3} and R^{D4} are H.

42. The compound of statements 38 or 39 or a pharmaceutically acceptable salt thereof, wherein R^{D1}, R^{D2}, R^{D3} and R^{D4} are H.

43. The compound of statement 38 or 39 or a pharmaceutically acceptable salt thereof, wherein R^{D3} is selected from the group consisting of: H; optionally substituted phenyl, wherein the optional substituent is halo, methyl optionally substituted by —S(═O)₂CH₃, OMe, C(═O)OH, Cl, CN; or piperazinyl optionally substituted by methyl; or pyrazolyl optionally substituted by methyl, triazolyl, or tetrazolyl, and wherein R^{D1}, R^{D2} and R^{D4} are all H.

44. The compound of statement 38 or 39 or a pharmaceutically acceptable salt thereof, wherein R^{D3} is selected from the group consisting of: H; optionally substituted phenyl, wherein the optional substituent is halo, methyl, OMe, C(═O)OH, Cl, CN; or piperazinyl optionally substituted by methyl; or pyrazolyl, triazolyl, or tetrazolyl, and wherein R^{D1}, R^{D2} and R^{D4} are all H.

45. The compound of statements 38 or 39 or a pharmaceutically acceptable salt thereof, wherein R^{D1} is selected from H, methyl, OMe, Cl, CF₃, OCF₃, pyrazolyl optionally substituted by methyl, triazolyl and CN, and wherein R^{D2}, R^{D3} and R^{D4} are all H.

46. The compound of statements 38 or 39 or a pharmaceutically acceptable salt thereof, wherein R^{D1} is selected from H, methyl, OMe, Cl, F, CF₃, OCF₃, and CN, and wherein R^{D2}, R^{D3} and R^{D4} are all H.

47. The compound of statement 38 or 39 or a pharmaceutically acceptable salt thereof, wherein R^{D3} and R^{D4} form an unsubstituted benzene ring or an unsubstituted pyridine ring.

48. The compound of statement 33 or 34 or a pharmaceutically acceptable salt thereof, wherein C is of the formula (C-1) and D is an optionally substituted phenyl or piperidyl, wherein there are one or two optional substituents selected from F, OMe and CN.

49. The compound of statement 33 or 34 or a pharmaceutically acceptable salt thereof, wherein D is of the formula (D-2):

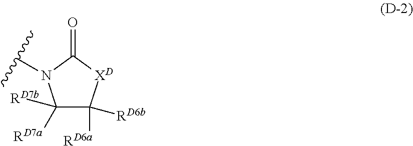

wherein X^{D} is NR^{D5a} or CR^{D5a}R^{D5b};

R^{D5a} is selected from H or methyl;

either R^{D5b} and R^{D6b} are both H or together they are —CH₂—;

R^{D6a} is selected from H, ═O, methyl, —CH₂OH or —C(═O)OH, wherein when R^{D6a} is ═O, R^{D6b} is absent;

R^{D7a} is selected from H, ═O, methyl, —CH₂OH or —C(═O)OH;

R^{D7b} is H, wherein when R^{D7a} is ═O, R^{D7b} is absent;

or wherein R^{D6a} and R^{D7a} together form a benzene ring or a C heteroaromatic ring which is optionally substituted by CN, P(═O)Me₂ or carboxy and R^{D6b} and R^{D7b} are absent.

50. The compound of statement 49 or a pharmaceutically acceptable salt thereof, wherein:
a) when R^{D6a} and R^{D6b} are H, R^{D7a} is selected from ═O, carboxy or CH₂OH and R^{D7b} is H or when R^{D7}a is ═O, R^{D7b} is absent;
b) when R^{D7a} is H, R^{D7b} is H, R^{D6a} is ═O and R^{D6b} is absent.

51. The compound of statement 49 or a pharmaceutically acceptable salt thereof, wherein R^{D6a} and R^{D7a} together form a benzene ring or a C₆ heteroaromatic ring which is optionally substituted by CN, C(═O)OH or P(═O)Me₂ and R^{D6b} and R^{D7b} are absent.

52. The compound of statement 49 or a pharmaceutically acceptable salt thereof, wherein R^{D6a} and R^{D7a} form an unsubstituted benzene ring or an unsubstituted pyridine ring and R^{D1b} and R^{D7b} are absent.

53. The compound of statement 33 or 34 or a pharmaceutically acceptable salt thereof, wherein 0 is selected from the following groups:

| Name | Structure |
|---|---|
| 2-oxo-1-pyridyl | 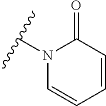 [SMILES: *n1ccccc1=O] |
| 3-methyl-2-oxo-1-pyridyl | 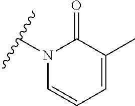 [SMILES: *n1cccc(C)c1=O] |
| 3-methoxy-2-oxo-1-pyridyl | 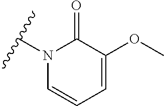 [SMILES: *n1cccc(OC)c1=O] |
| 5-methoxy-2-oxo-1-pyridyl | 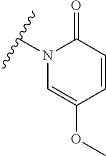 [SMILES: *n1cc(OC)ccc1=O] |
| 5-chloro-2-oxo-1-pyridyl | 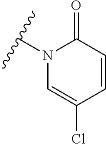 [SMILES: *n1cc(Cl)ccc1=O] |
| 2-oxo-1,8-naphthyridin-1-yl | 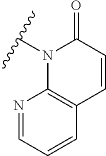 [SMILES: *n1c(=O)ccc2cccnc21] |
| 2-oxo-1-quinolyl | 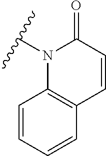 [SMILES: *n1c(=O)ccc2ccccc21] |
| 3-methyl-2-oxo-benzimidazol- 1-yl | 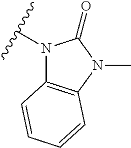 [SMILES: *n1c(=O)n(C)c2ccccc21] |
| 3-methyl-2,4-dioxo-imidazol- idin-1-yl | 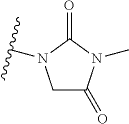 [SMILES: *N1CC(=O)N(C)C1=O] |
| 3-methyl-2,5-dioxo-imidazol- idin-1-yl | 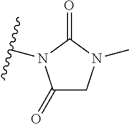 [SMILES: *N1C(=O)CN(C)C1=O] |
| 3-chloro-2-oxo-1-pyridyl | 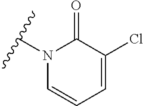 [SMILES: *n1cccc(Cl)c1=O] |
| 3-cyano-2-oxo-1-pyridyl | 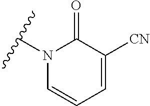 [SMILES: *n1cccc(C#N)c1=O] |
| 5-cyano-2-oxo-1-pyridyl | 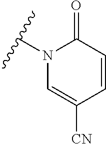 [SMILES: *n1cc(C#N)ccc1=O] |
| 3-fluoro-2-oxopyridin-1(2H)-yl | |
| 5-methyl-2-oxo-1-pyridyl | |
| 5-carboxy-2-oxo-1-pyridyl | |
| 2-oxo-5-(1H-pyrazol-4-yl)-1- pyridyl | 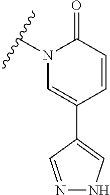 [SMILES: *n1cc(-c2cn[nH]c2)ccc1=O] |
| 2-oxo-5-(1H-triazol-4-yl)-1- pyridyl | 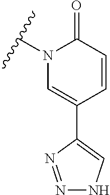 [SMILES: *n1cc(-c2c[nH]nn2)ccc1=O] |
| 5-cyano-3-methyl-2-oxo- benzimidazol-1-yl | 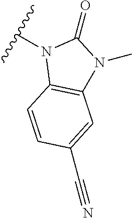 [SMILES: *n1c(=O)n(C)c2cc(C#N)ccc21] |
| 5-carboxy-3-methyl-2-oxo- benzimidazol-1-yl | 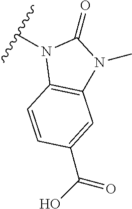 [SMILES: *n1c(=O)n(C)c2cc(C(=O)O)ccc21] |
| 6-carboxy-3-methyl-2-oxo- benzimidazol-1-yl | 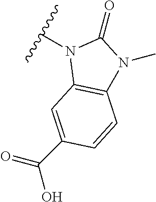 [SMILES: *n1c(=O)n(C)c2ccc(C(=O)O)cc21] |
| (6-dimethylphosphoryl-3- methyl-2-oxo-benzimidazol- 1-yl) | 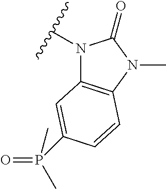 [SMILES: *n1c(=O)n(C)c2ccc(P(C)(C)=O)cc21] |
| 2-oxo-3H-benzimidazol- 1-yl | 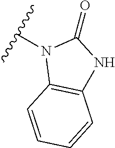 [SMILES: *n1c(=O)[nH]c2ccccc21] |
| 2-oxo-1H-imidazo[4,5-b]- pyridin-3-yl | 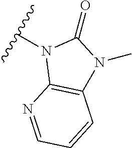 [SMILES: *n1c(=O)n(C)c2cccnc21] |
| 6-oxopyridazin-1-yl | 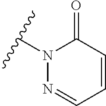 [SMILES: *n1ncccc1=O] |
| 6-oxopyrimidin-1-yl | 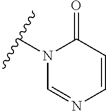 [SMILES: *n1cnccc1=O] |
| 1-methyl-2-oxo-3-pyridyl | 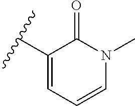 [SMILES: *c1cccn(C)c1=O] |
| 2-oxo-3-(trifluoromethyl)-1- pyridyl | 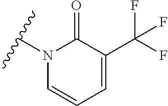 [SMILES: *n1cccc(C(F)(F)F)c1=O] |
| 2-oxo-3-(trifluoromethoxy)- 1-pyridyl | 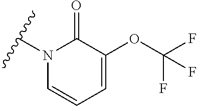 [SMILES: *n1cccc(OC(F)(F)F)c1=O] |
| 2-oxo-5-(1H-tetrazol-5-yl)- 1-pyridyl | 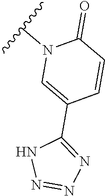 [SMILES: *n1cc(-c2nnn[nH]2)ccc1=O] |

54. The compound of statement 33 or 34 or a pharmaceutically acceptable salt thereof, wherein D is selected from the following groups:

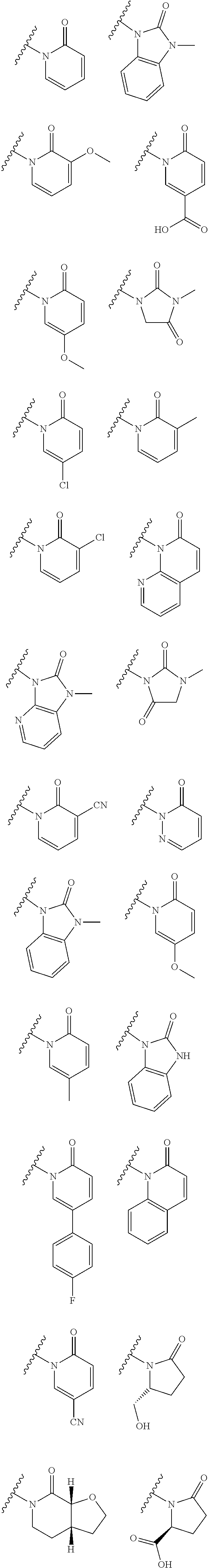

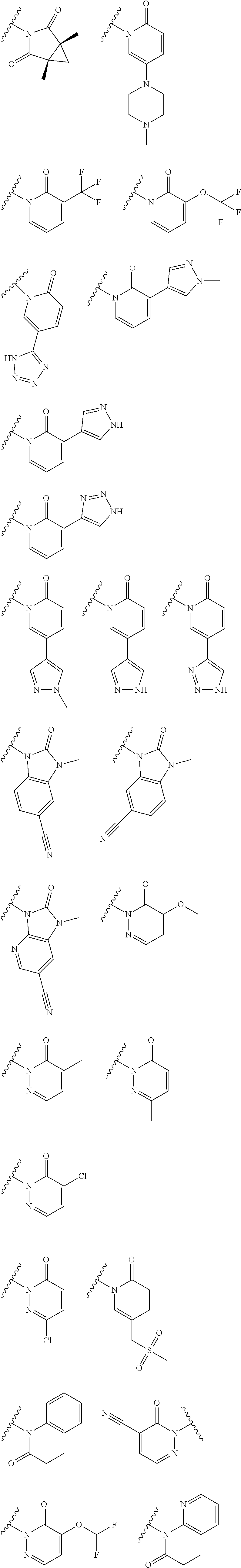

55. The compound of any one of statements 1 to 25 or a pharmaceutically acceptable salt thereof, wherein C is of the formula (C-2):

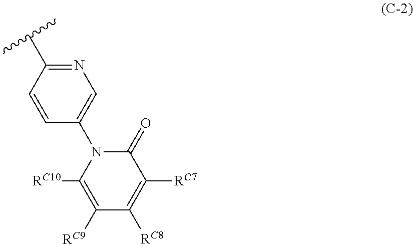

wherein one of R^{C7}, R^{C8}, R^{C9} and R^{C10} are selected from the group consisting of: methyl optionally substituted with S(═O)₂CH₃; OMe; piperazinyl optionally substituted by methyl; C(═O)OH (carboxy); C₁; F; pyrazolyl optionally substituted with methyl; triazolyl; tetrazole; optionally substituted phenyl (wherein the optional substituent is methyl or halo); CN; CF₃; O—CF₃; and the rest of R^{C7}, R^{C8}, R^{C9} and R^{C10} are H; or

R^{C9} and R^{C10} form a benzene or 6 membered heteroaromatic ring, and R^{C7} and R^{C8} are both H; or wherein R^{C7}, R^{C8}, R^{C9} and R^{C10} are all H.

56. The compound of any one of statements 1 to 25 or a pharmaceutically acceptable salt thereof, wherein C is of the formula (C-2):

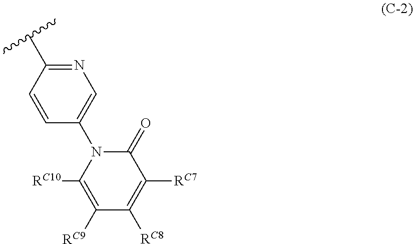

wherein one of R^{C7}, R^{C8}, R^{C9} and R^{C10} are selected from the group consisting of: methyl, OMe; piperazinyl optionally substituted by methyl; C(═O)OH (carboxy); C₁; F; pyrazolyl; triazolyl; tetrazole; optionally substituted phenyl (wherein the optional substituent is methyl or halo); CN; CF₃; O—CF₃; and the rest of R^{C7}, R^{C8}, R^{C9} and R^{C10} are H; or

R^{C9} and R^{C10} form a benzene or 6 membered heteroaromatic ring, and R^{C7} and R^{C8} are both H; or wherein R^{C7}, R^{C8}, R^{C9} and R^{C10} are all H.

57. The compound of statement 55 or 56 or a pharmaceutically acceptable salt thereof, wherein:
a) all of R^{C7}, R^{C8}, R^{C9} and R^{C10} are H; or
b) R^{C9} is selected from H, optionally substituted phenyl (wherein the optional substituent is F), methyl optionally substituted with —S(═O)₂CH₃, OMe, C(═O)OH, Cl, pyrazolyl optionally substituted by methyl, triazolyl, tetrazolyl, CN and piperazinyl optionally substituted by methyl, and R^{C7}, R^{C8} and R^{C10} are all H; or
c) R^{C7} is selected from H, methyl, OMe, O—CF₃, CF₃, Cl, pyrazolyl optionally substituted by methyl, triazolyl and CN and R^{C8}, R^{C9} and R^{C10} are all H.

58. The compound of statement 55 or 56 or a pharmaceutically acceptable salt thereof, wherein:
a) all of R^{C7}, R^{C8}, R^{C9} and R^{C10} are H; or
b) R^{C9} is selected from H, optionally substituted phenyl (wherein the optional substituent is F), methyl, OMe, C(═O)OH, Cl, F, pyrazolyl, triazolyl, tetrazolyl, CN and piperazinyl optionally substituted by methyl, and R^{C7}, R^{C8} and R^{C10} are all H; or
c) R^{C7} is selected from H, methyl, OMe, O—CF₃, CF₃, Cl, F and CN and R^{C8}, R^{C9} and R^{C10} are all H.

59. The compound of any one of statements 1 to 25 or a pharmaceutically acceptable salt thereof, wherein C is selected from the group consisting of:

| No. | Structure |
|---|---|
| 1 | 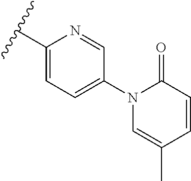 [SMILES: *c1ccc(-n2cc(C)ccc2=O)cn1] |
| 2 | 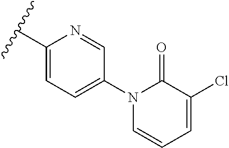 [SMILES: *c1ccc(-n2cccc(Cl)c2=O)cn1] |
| 3 | 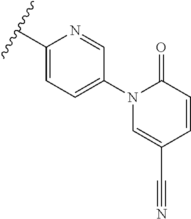 [SMILES: *c1ccc(-n2cc(C#N)ccc2=O)cn1] |
| 4 | 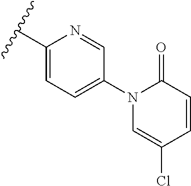 [SMILES: *c1ccc(-n2cc(Cl)ccc2=O)cn1] |
| 5 | 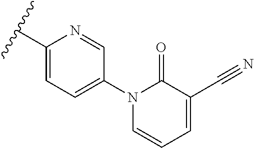 [SMILES: *c1ccc(-n2cccc(C#N)c2=O)cn1] |
| 6 | 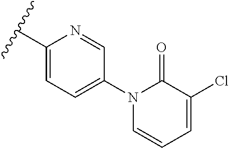 [SMILES: *c1ccc(-n2cccc(Cl)c2=O)cn1] |
| 7 | 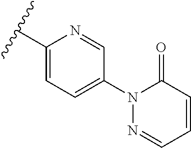 [SMILES: *c1ccc(-n2ncccc2=O)cn1] |
| 8 | 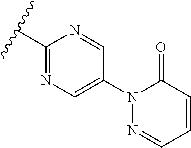 [SMILES: *c1ncc(-n2ncccc2=O)cn1] |
| 9 | 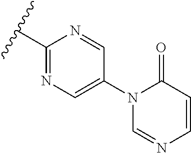 [SMILES: *c1ncc(-n2cnccc2=O)cn1] |
| 10 | 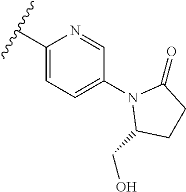 |
| 11 | 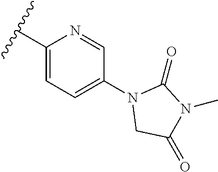 [SMILES: *c1ccc(N2CC(=O)N(C)C2=O)cn1] |
| 12 | 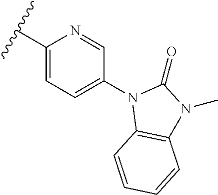 [SMILES: *c1ccc(-n2c(=O)n(C)c3ccccc32)cn1] |
| 13 | 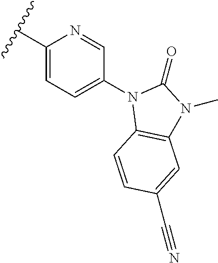 [SMILES: *c1ccc(-n2c(=O)n(C)c3cc(C#N)ccc32)cn1] |
| 14 | 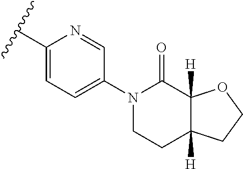 [SMILES: *c1ccc(N2CC[C@H]3CCO[C@H]3C2=O)cn1] |
| 15 | 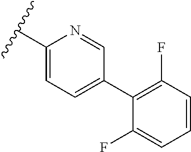 [SMILES: *c1ccc(-c2c(F)cccc2F)cn1] |
| 16 | 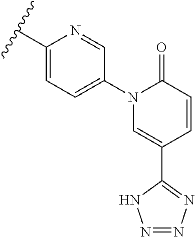 [SMILES: *c1ccc(-n2cc(-c3nnn[nH]3)ccc2=O)cn1] |
| 17 | 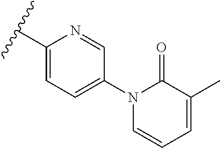 [SMILES: *c1ccc(-n2cccc(C)c2=O)cn1] |
| 18 | 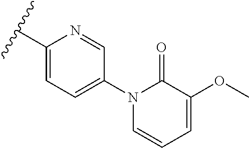 [SMILES: *c1ccc(-n2cccc(OC)c2=O)cn1] |
| 19 | 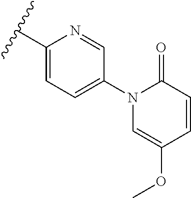 [SMILES: *c1ccc(-n2cc(OC)ccc2=O)cn1] |
| 20 | 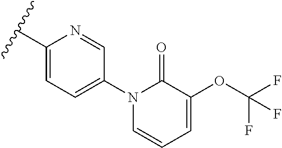 [SMILES: *c1ccc(-n2cccc(OC(F)(F)F)c2=O)cn1] |
| 21 | 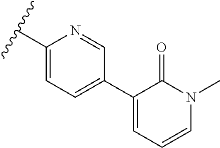 [SMILES: *c1ccc(-c2cccn(C)c2=O)cn1] |
| 22 | 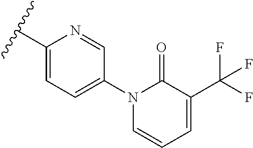 [SMILES: *c1ccc(-n2cccc(C(F)(F)F)c2=O)cn1] |
| 23 | 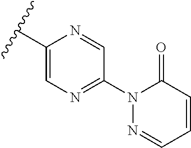 [SMILES: *c1cnc(-n2ncccc2=O)cn1] |
| 24 | 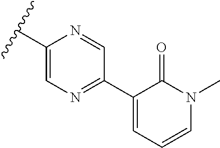 [SMILES: *c1cnc(-c2cccn(C)c2=O)cn1] |
| 25 | 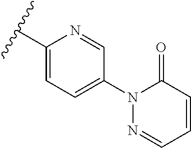 [SMILES: *c1ccc(-n2ncccc2=O)cn1] |
| 26 | 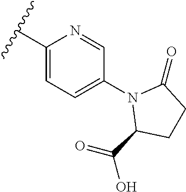 [SMILES: *c1ccc(N2C(=O)CC[C@H]2C(=O)O)cn1] |
| 27 | 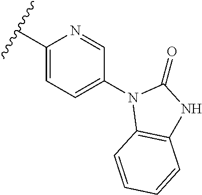 [SMILES: *c1ccc(-n2c(=O)[nH]c3ccccc32)cn1] |
| 28 | 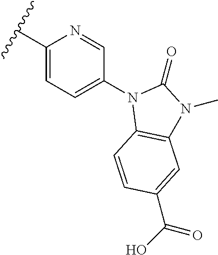 [SMILES: *c1ccc(-n2c(=O)n(C)c3cc(C(=O)O)ccc32)cn1] |
| 29 | 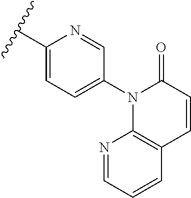 [SMILES: *c1ccc(-n2c(=O)ccc3cccnc32)cn1] |
| 30 | 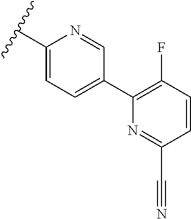 [SMILES: *c1ccc(-c2nc(C#N)ccc2F)cn1] |
| 31 | 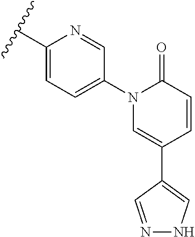 [SMILES: *c1ccc(-n2cc(-c3cn[nH]c3)ccc2=O)cn1] |
| 32 | 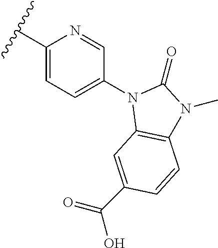 [SMILES: *c1ccc(-n2c(=O)n(C)c3ccc(C(=O)O)cc32)cn1] |
| 33 | 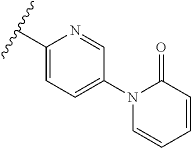 [SMILES: *c1ccc(-n2ccccc2=O)cn1] |
| 34 | 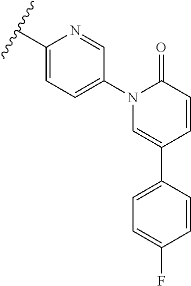 [SMILES: *c1ccc(-n2cc(-c3ccc(F)cc3)ccc2=O)cn1] |
| 35 | 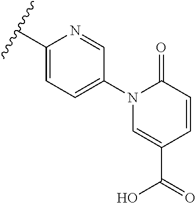 [SMILES: *c1ccc(-n2cc(C(=O)O)ccc2=O)cn1] |
| 36 | 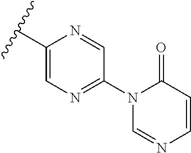 [SMILES: *c1cnc(-n2cnccc2=O)cn1] |
| 37 | 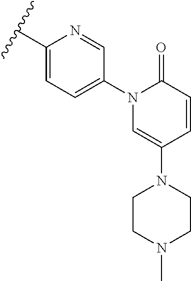 [SMILES: *c1ccc(-n2cc(N3CCN(C)CC3)ccc2=O)cn1] |
| 38 | 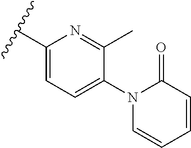 [SMILES: *c1ccc(-n2ccccc2=O)c(C)n1] |
| 39 | 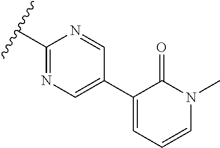 [SMILES: *c1ncc(-c2cccn(C)c2=O)cn1] |
| 40 | 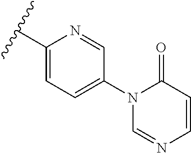 [SMILES: *c1ccc(-n2cnccc2=O)cn1] |
| 41 | 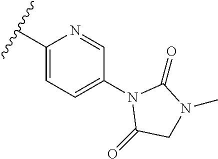 [SMILES: *c1ccc(N2C(=O)CN(C)C2=O)cn1] |
| 42 | 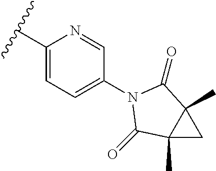 [SMILES: *c1ccc(N2C(=O)[C@]3(C)C[C@]3(C)C2=O)cn1] |
| 43 | 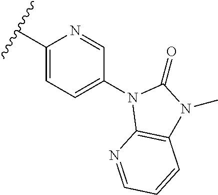 [SMILES: *c1ccc(-n2c(=O)n(C)c3cccnc32)cn1] |
| 44 | 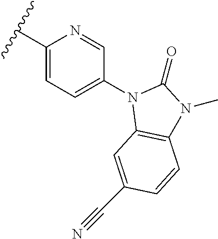 [SMILES: *c1ccc(-n2c(=O)n(C)c3ccc(C#N)cc32)cn1] |
| 45 | 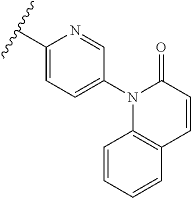 [SMILES: *c1ccc(-n2c(=O)ccc3ccccc32)cn1] |
| 46 | 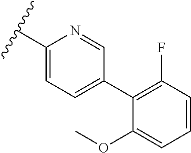 [SMILES: *c1ccc(-c2c(F)cccc2OC)cn1] |
| 47 | 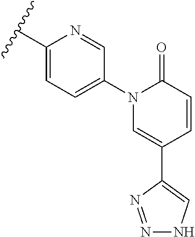 [SMILES: *c1ccc(-n2cc(-c3c[nH]nn3)ccc2=O)cn1] |
| 48 | 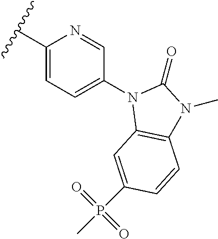 |

60. The compound of any one of statements 1 to 25 or a pharmaceutically acceptable salt thereof, wherein C is selected from the group consisting of:

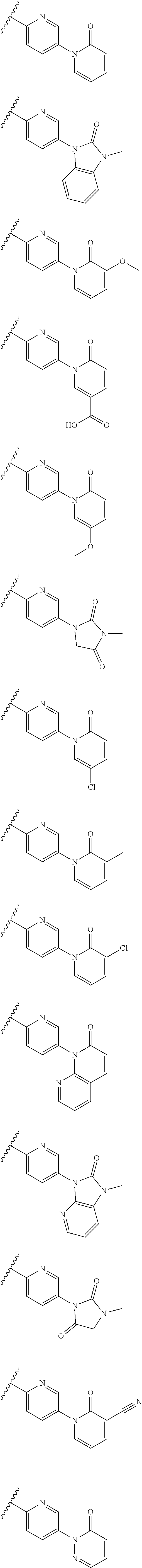

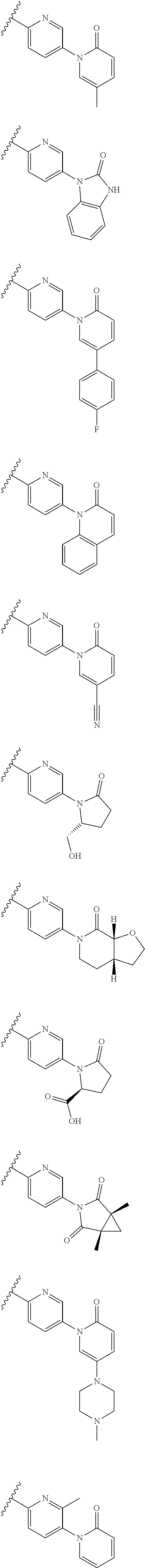

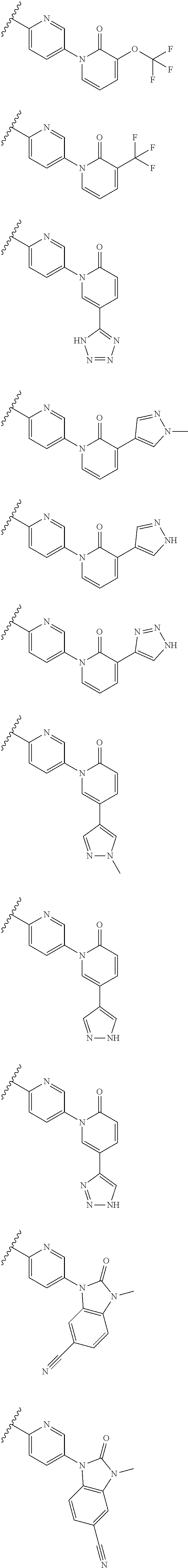

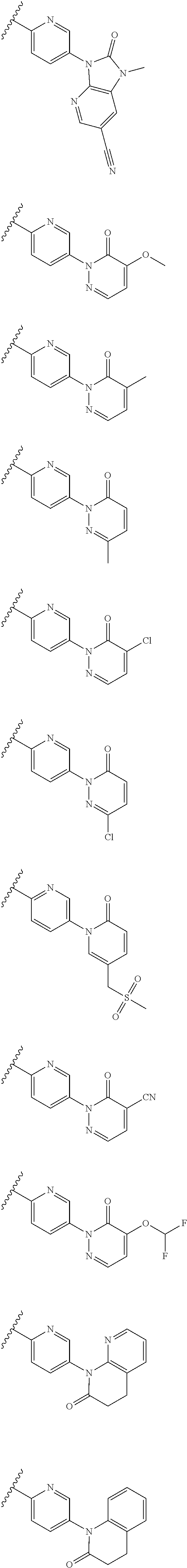

61. The compound of any of the preceding statements or a pharmaceutically acceptable salt thereof, wherein A-B—C is of the formula (I-A), (I-B), (I-Ba), (I-Bb), (I-C), or (I-D):

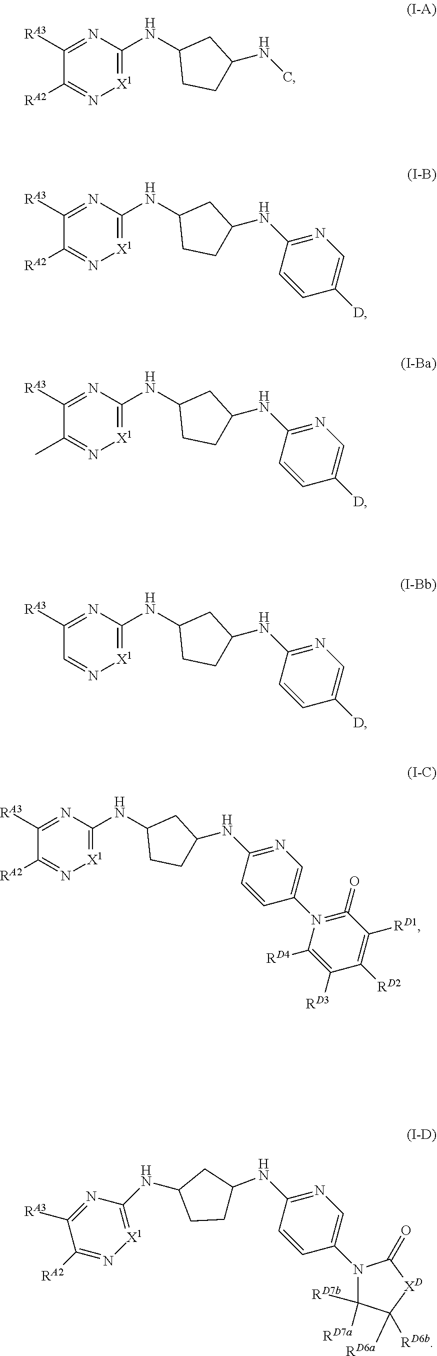

wherein X¹, R^{A2}, R^{A3}, C, D, R^{D1}, R^{D2}, R^{D3}, R^{D4}, R^{D1a}, R^{D2a}, R^{D3a}, R^{D4a}, X^{D}, R^{D6a}, R^{D6b}, R^{D7a} and R^{D7b} are as defined in any of the preceding statements.

62. The compound of Table 1, Table 2 or Table 2a or a pharmaceutically acceptable salt thereof.

63. The compound of any one of statements 1 to 62 or a pharmaceutically acceptable salt thereof, for use in therapy.

64. A pharmaceutical composition comprising the compound of any one of statements 1 to 62 or a pharmaceutically acceptable salt thereof, and a pharmaceutically acceptable diluent, carrier or excipient.

65. The compound of any one of statements 1 to 62 or a pharmaceutically acceptable salt thereof, or a pharmaceutical composition according to statement 64 for use in the treatment of a cardiovascular disease.

66. The compound for use according to statements 65 wherein the compound is administered simultaneously, separately or sequentially in combination with an additional active ingredient selected from the group consisting of:
i) a statin;
ii) a cholesterol absorption inhibitor;
iii) a SGLT2 inhibitor;
iv) a P2Y12 inhibitor;
v) a citrate lyase inhibitor; and
vi) anti-hypertensive drugs.

67. The compound use according to statements 65 or 66 when the cardiovascular disease is selected from dyslipidemia, hypercholesterolemia, hypertriglyceridemia, hyperlipidemia, hypoalphalipoproteinemia, metabolic syndrome, diabetic complications, atherosclerosis, stroke, vascular dimensia, chronic kidney disease, coronary heart disease, coronary artery disease, retinopathy, inflammation, thrombosis, peripheral vascular disease heart failure or congestive heart failure.

68. Use of a compound of any one of statements 1 to 62 or a pharmaceutically acceptable salt thereof, or a pharmaceutical composition according to statement 64 in a method of medical treatment.

69. A method of medical treatment comprising administering to the patient the pharmaceutical composition of statement 64.

70. Use of a compound of any one of statements 1 to 62 in the manufacture of a medicament for use in therapy.

71. A method of treating PCSK9-mediated disease or disorder in a patient need thereof comprising administering to the patient a therapeutically effective amount of the compound or pharmaceutically acceptable salt thereof according to any one of statements 1 to 62 or the pharmaceutical composition according to statement 64.

72. The method according to statement 71, wherein the disease or disorder is a cardiovascular disease or disorder.

73. The method according to statements 72, wherein the cardiovascular disease or disorder is selected from dyslipidemia, hypercholesterolemia, hypertriglyceridemia, hyperlipidemia, hypoalphalipoproteinemia, metabolic syndrome, diabetic complications, atherosclerosis, stroke, vascular dimensia, chronic kidney disease, coronary heart disease, coronary artery disease, retinopathy, inflammation, thrombosis, peripheral vascular disease heart failure or congestive heart failure.

1. A compound of Formula (I)
A-B—C (I)
or a pharmaceutically acceptable salt, tautomeric forms or stereoisomers thereof,
wherein A is of the following: 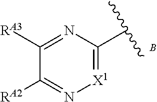
wherein the wavy line indicates the point of attachment to B;
X¹ is C—R^{A1};
R^{A1} is selected from the group consisting of:
(i) H;
(ii) halo;
(iii) CN;
(iv) C₁₋₆ hydrocarbon, optionally substituted by one or more OH, CN, C₁₋₆ acyl, C₁₋₆ alkoxy or one or more halo groups;
(v) C₁₋₆ alkoxy, optionally substituted by OH, one or more halo groups, C₁₋₆ alkyl amido;
(vi) C₁₋₆ alkylester;
(vii) C₁₋₆ alkyl acyl; and
(viii) OH;
R^{A2} is selected from the group consisting of:
(i) H;
(ii) halo;
(iii) CN;
(iv) C₁₋₆ hydrocarbon, optionally substituted by OH, CN, C₁₋₆acyl, C₁₋₆alkoxy or one or more halo groups;
(v) C₁₋₆ alkoxy, optionally substituted by OH, alkyl amido, or one or more halo groups;
(vi) C₁₋₆ acyl amido (wherein the acyl is optionally substituted by H or methyl);
(vii) C₁₋₆ thioalkyl optionally substituted by C₁₋₆ alkyl ester;
(viii) C₁₋₆ alkyl ester;
(ix) C₁₋₆ alkyl acyl;
(x) C₄₋₅ heterocyclyl;
(xi) C₅ heteroaryl;
(xii) C₁₋₆ alkyl amido optionally substituted by C₁₋₃ alkyl amido, CN, OH, C₂₋₃ alkynyl, C₄₋₆heterocyclyl or C₁₋₃alkyl wherein the C₁₋₃alkyl is optionally substituted with one or more halo or OH groups; and
(xiii) OH;
R^{A3} is selected from the group consisting of:
(i) H;
(ii) halo;
(iii) CN;
(iv) C₁₋₆ hydrocarbon optionally substituted by OH, CN, C₁₋₆ thioalkyl, C₁₋₆ alkoxy, C₁₋₆ alkyl acyl, C₁₋₆ acyloxy, carboxy, C₁₋₆ alkyl ester, C₁₋₆ alkylamino, —C(═O)NH₂, C₁₋₆ alkyl amido, C₁₋₆ alkyl acylamido, C₁₋₆ alkyl sulfinyl, C₁₋₆ alkyl sulfonyl or one or more halo groups;
(v) OH;
(vi) C₁₋₆ alkoxy, optionally substituted by OH, NH₂, C₄ heterocyclyl or one or more halo groups;
(vii) C₁₋₆ acyloxy;
(viii) C₄ heterocycyl;
(ix) —NH₂;
(x) C₁₋₆ alkylamino, optionally substituted by CN, OH or C₄ heterocyclyl;
(xi) C₁₋₆ dialkylamino, optionally substituted by —NH₂;
(xii) C₁₋₆ acylamido (where acyl substituent is H or Me);
(xiii) carbaimidoyl or methyl-carbaimidoyl;
(xiv) carboxyamino;
(xv) C₁₋₆ thioalkyl, optionally substituted by OH or NH₂;
(xvi) C₁₋₆ alkyl sulfinyl;
(xvi) C₁₋₆ alkyl sulfonyl, optionally substituted by one or more halo groups;
(xvii) C₁₋₆ sulfonimodyl;
(xviii) C₁₋₆ alkyl phosphinyl;
(xix) carboxy;
(xx) C(═O)NH₂
(xxi) C₁₋₆ alkyl ester;
(xxii) C₁₋₆ alkyl acyl, optionally substituted by one or more halo groups; and
(xxiii) C₁₋₆ alkyl amido;
or wherein R^{A3} and R^{A2} together with the carbon atoms to which they are bound form:
(i) an optionally substituted C₅₋₇ heterocycle ring;
(ii) an optionally substituted C₅₋₇ heteroaromatic ring;
(iii) an optionally substituted C₆ carboaromatic ring;
(iv) an optionally substituted C₅₋₇ carbocyclic ring
wherein the optional substituents are selected from C₁₋₆alkyl, halo, C₁₋₆ alkoxy, NH₂, C₁₋₆alkylamino, OH, and CN;
wherein B is of formula (B-1) or (B-2)
i) 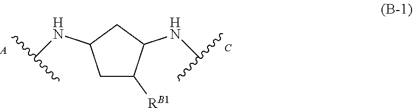
wherein the wavy line indicates the point of attachment to A and C;
R^{B1} is H, OH, ═CHCH₂—OH, —O—C₁₋₄alkyl or C₁₋₄alkyl, wherein the C₁₋₄alkyl is optionally substituted by OH or OMe;
(ii) 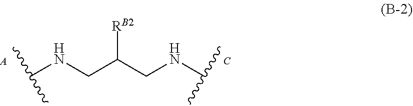
wherein the wavy line indicates the point o attachment to an;
R^{B2} is C₁₋₂ alkyl-OH, CH₂CONHMe or C₁₋₃ alkyl;
wherein C is selected from the group consisting of C₆₋₁₀ carboaryl, C₅₋₆ heteroaryl and C₅₋₁₀ heterocyclyl, which groups are optionally substituted by:
(i) C₆₋₁₀ carboaryl, C₄₋₁₀ carbocyclyl, C₅₋₁₀ heteroaryl, C₄₋₁₀ heterocyclyl, or C₅₋₁₀ bridged heterocycle, spiro C₆₋₁₂heterocyclyl or a spiro C₆₋₁₂carbocyclyl, which are themselves optionally substituted by one or more of the following groups:
a) one or two ═O groups;
b) one or more halo groups;
c) CN, NH₂, OH;
d) one or more C₁₋₆ alkyl groups including branched and cyclic and with an optional substituent selected from OH, C₁₋₆ alkyl sulfonyl or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl optionally substituted with methyl;
i) C₄₋₁₀ carbocyclyl with an optional methyl or ═O substituent;
j) C₆₋₁₀ carboaryl with optional substituents of one or more halo groups;
l) P(═O)Me₂;
m) carboxy or CH₂-carboxy;
n) tetrazolyl, CH₂-tetrazolyl, 5-oxo-4H-1,2,4-oxadiazol-3-yl;
(ii) one or more groups selected from carboxy, CN, halo, nitro, C₁₋₆ alkyl, C₁₋₆ thioalkyl, C₁₋₆ alkoxy, C₁₋₆ alkylacyl, C₁₋₆ alkyl amido, di-C₁₋₆ alkyl amido, C₁₋₆ alkyl sulfonamido, and di-C₁₋₆ alkyl sulfonamido.

2. The compound of claim 1 or a pharmaceutically acceptable salt thereof, wherein R^{A1} is selected from the group consisting of:
(i) H;
(ii) halo;
(iii) CN;
(iv) C₁₋₆ hydrocarbon, optionally substituted by OH, CN, C₁₋₆ acyl, C₁₋₆ alkoxy or one or more halo groups;
(v) C₁₋₆ alkoxy, optionally substituted by OH, one or more halo groups, C₁₋₆ alkyl amido; and
(vi) OH.

3. The compound of claim 1 or a pharmaceutically acceptable salt thereof, wherein R^{A1} is H or OH.

4. The compound of any one of claim 1 or a pharmaceutically acceptable salt thereof, wherein R^{A2} is selected from the group consisting of:
(i) H;
(ii) halo;
(iii) CN;
(iv) C₁₋₆ hydrocarbon, optionally substituted by OH, CN, C₁₋₆acyl, C₁₋₆alkoxy or one or more halo groups;
(v) OH;
(vi) C₁₋₆ alkoxy, optionally substituted by OH, C₁₋₆ alkyl amido, or one or more halo groups;
(vii) C₁₋₆ alkyl ester;
(viii) C₁₋₆ alkyl acyl;
(ix) C₁₋₆ alkyl amido optionally substituted by C₁₋₃ alkyl amido, CN, C₂₋₃ alkynyl, C₄₋₆ heterocyclyl, or C₁₋₃ alkyl which alkyl is optionally substituted with one or more halo or OH groups; and
(x) C₁₋₆ thioalkyl optionally substituted by C₁₋₆ alkyl ester.

5. The compound of claim 1 or a pharmaceutically acceptable salt thereof, wherein R^{A2} is selected from the group consisting of CN, methyl, Cl, —C(═O)CH₃, OCHF₂, cyclopropyl, OCF₃, OCH₃, H, —C(═O)NH(CH₃), S—CH₃, —S—CH₂CH₃ or —S—CH₂—C(═O)—O—CH₃.

6. The compound of claim 1 or a pharmaceutically acceptable salt thereof, wherein R^{A3} is selected from the group consisting of:
(i) H;
(ii) halo;
(iii) CN;
(iv) C₁₋₆ hydrocarbon, optionally substituted by OH, CN, C₁₋₆ thioalkyl, C₁₋₆ alkoxy, C₁₋₆ alkyl acyl C₁₋₆ acyloxy, carboxy, C₁₋₆ alkylester, C₁₋₆ alkylamino; —C(═O)NH₂, C₁₋₆ alkyl amido, C₁₋₆ alkylacylamido, C₁₋₆ alkyl sulfinyl, C₁₋₆ alkyl sulfonyl or one or more halo groups;
(v) OH; and
(vi) C₁₋₆ alkoxy, optionally substituted by OH, NH₂, C₄ heterocyclyl or one or more halo groups.

7. The compound of claim 1 or a pharmaceutically acceptable salt thereof, wherein R^{A3} is selected from the group consisting of H, CN and methyl.

8. The compound of claim 1 or a pharmaceutically acceptable salt thereof, wherein R^{A3} and R^{A2} together with the carbon atoms to which they are bound form an optionally substituted C₆ carboaromatic ring or C₅ heteroaromatic ring, wherein the optional substituents are selected from methyl, NH₂, Cl, F and OMe.

9. The compound of claim 1 or a pharmaceutically acceptable salt thereof, wherein B is of formula (B-1) 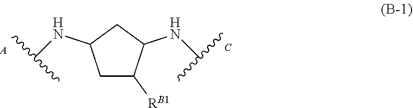
wherein the wavy line indicates the point of attachment to A and C;
wherein R^{B1} is selected from the group consisting of H, OH, OMe, —O-Ethyl, —CH₂OH, —CH₂CH₂OH and ═CHCH₂—OH.

10. The compound of claim 1 or a pharmaceutically acceptable salt thereof, wherein B is of the following formula (B-1a):

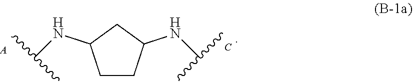

11. The compound of claim 1 or a pharmaceutically acceptable salt thereof, wherein C is an optionally substituted pyridinyl, pyrazinyl or pyrimidinyl, wherein the optional substituents are selected from C₆₋₁₀ carboaryl, C₄₋₁₀ carbocyclyl, C₅₋₁₀ heteroaryl, C₅₋₁₀ heterocyclyl, C₅₋₁₀ bridged heterocyclyl, spiro C₆₋₁₂heterocyclyl and a spiro C₆₋₁₂ carbocyclyl, which are themselves optionally substituted by one or more of the following groups:
a) one or two ═O groups;
b) one or more halo groups;
c) CN, NH₂ or OH;
d) one or more C₁₋₆ alkyl groups including branched and cyclic and with an optional substituent selected from OH, C₁₋₆ alkyl sulfonyl or one or more halo groups;
e) C₁₋₆ alkoxy with optional substituents of one or more halo groups;
f) C₁₋₆ alkylester;
g) C₅₋₆ heterocyclyl with an optional methyl, OH or ═O substituent;
h) C₅₋₆ heteroaryl with an optional methyl substituent;
i) C₄₋₁₀ carbocyclyl with an optional methyl or ═O substituent;
j) C₆₋₁₀ carboaryl with optional substituents of one or more halo groups;
l) P(═O)Me₂;
m) carboxy or CH₂-carboxy; and/or
n) tetrazolyl, CH₂-tetrazolyl, 5-oxo-4H-1,2,4-oxadiazol-3-yl.

12. The compound of claim 1 or a pharmaceutically acceptable salt thereof, wherein C is of the formula (C-1): 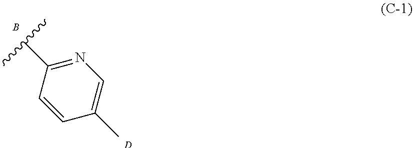
wherein D is C₆₋₁₀carboaryl, C₅₋₁₀ heteroaryl or C₅₋₁₀ heterocyclyl, each which are themselves optionally substituted by:
i) one or two ═O groups;
ii) one or two C₁₋₄ alkyl groups which can be branched, optionally substituted by S(═O)₂CH₃;
iii) OMe;
iv) piperazinyl, optionally substituted by methyl;
v) C(═O)OH (carboxy);
vi) Cl;
vii) F;
viii) phenyl, optionally substituted by one or more fluoro;
ix) CN;
x) CF₃;
xi) O—CF₃;
xii) O—CF₂;
xiii) pyrazolyl optionally substituted by methyl, triazolyl; tetrazolyl;
xiv) NH₂;
xv) pyridinyl;
xvi) CH₂OH;
xvii) OH; or
xviii) P(═O)Me₂.

13. The compound of claim 12 or a pharmaceutically acceptable salt thereof, wherein D is of the formula (D-1): 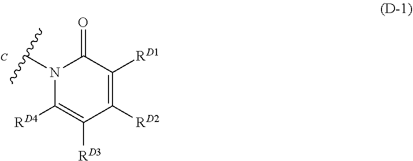
wherein one or two of R^{D1}, R^{D2}, R^{D3} and R^{D4} are selected from
i) C₁₋₆ alkyl, optionally substituted by C₁₋₆ alkyl sulfonyl or one or more halo groups;
ii) C₁₋₆ alkoxy, optionally substituted by one or more halo groups;
iii) C₅₋₆ heterocyclyl or C₅₋₆ heteroaryl optionally substituted with methyl substituent;
iv) carboxy or CH₂-carboxy;
v)═O, halo, NH₂ or CN;
vi) phenyl, optionally substituted by one or more halo atoms;
and the rest are H; or
wherein R^{D3} and R^{D4} form an optionally substituted 6 membered carboaromatic, heterocycle or heteroaromatic ring, wherein the optional substituents are selected from OH, methyl, OMe, halo and C(═O)OH;
or wherein R^{D1}, R^{D2}, R^{D3} and R^{D4} are all H.

14. The compound of claim 13 or a pharmaceutically acceptable salt thereof, wherein:
a) R^{D1}, R^{D2}, R^{D3} and R^{D4} are H; or
b) R^{D3} is selected from the group consisting of: H; optionally substituted phenyl, wherein the optional substituent is halo, methyl optionally substituted by —S(═O)₂CH₃, OMe, C(═O)OH, Cl, CN; or piperazinyl optionally substituted by methyl; or pyrazolyl optionally substituted by methyl, triazolyl, or tetrazolyl, and wherein R^{D1}, R^{D2} and R^{D4} are all H; or
c) R^{D1} is selected from H, methyl, OMe, Cl, CF₃, OCF₃, pyrazolyl optionally substituted by methyl, triazolyl and CN, and wherein R^{D2}, R^{D3} and R^{D4} are all H; or
d) R^{D3} and R^{D4} form an unsubstituted benzene ring or an unsubstituted pyridine ring.

15. The compound of claim 12 or a pharmaceutically acceptable salt thereof, wherein D is of the formula (D-2): 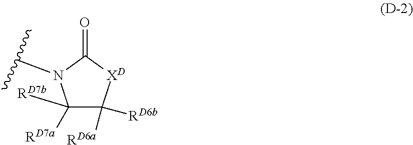
wherein X^{D} is NR^{D5a} or CR^{D5a}R^{D5b};
R^{D5a} is selected from H or methyl;
either R^{D5b} and R^{D6b} are both H or together they are —CH₂—;
R^{D6a} is selected from H, ═O, methyl, —CH₂OH or —C(═O)OH, wherein when R^{D6a} is ═O, R^{D6b} is absent;
R^{D7a} is selected from H, ═O, methyl, —CH₂OH or —C(═O)OH;
R^{D7b} is H, wherein when R^{D7}a is ═O, R^{D7b} is absent;
or wherein R^{D6a} and R^{D7a} together form a benzene ring or a C₆ heteroaromatic ring which is optionally substituted by CN, P(═O)Me₂ or carboxy and R^{D6b} and R^{D7b} are absent.

16. The compound of claim 12 or a pharmaceutically acceptable salt thereof, wherein D is selected from the following groups:

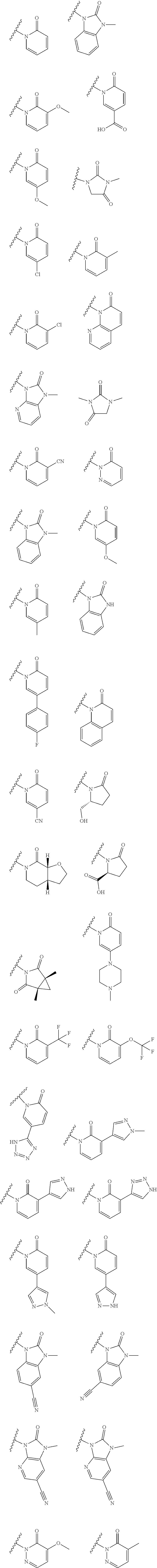

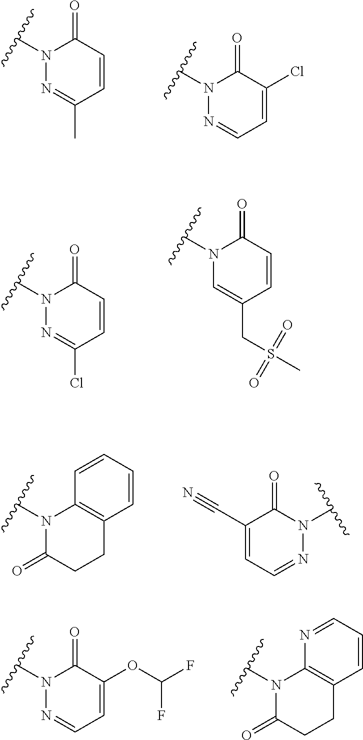

17. The compound of claim 1 or a pharmaceutically acceptable salt thereof, wherein C is of the formula (C-2): 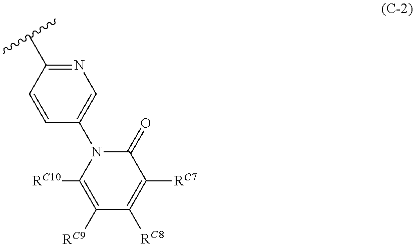
wherein one of R^{C7}, R^{C8}, R^{C9} and R^{C10} are selected from the group consisting of: methyl optionally substituted with S(═O)₂CH₃; OMe; piperazinyl optionally substituted by methyl; C(═O)OH (carboxy); Cl; F; pyrazolyl optionally substituted with methyl; triazolyl; tetrazole; optionally substituted phenyl (wherein the optional substituent is methyl or halo); CN; CF₃; O—CF₃; and the rest of R^{C7}, R^{C8}, R^{C9} and R^{C10} are H; or
R^{C9} and R^{C10} form a benzene or 6 membered heteroaromatic ring, and R^{C7} and R^{C8} are both H; or wherein R^{C7}, R^{C8}, R^{C9} and R^{C10} are all H.

18. The compound of claim 1 or a pharmaceutically acceptable salt thereof, wherein C is selected from the group consisting of:

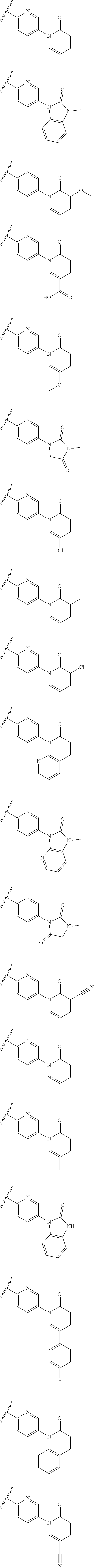

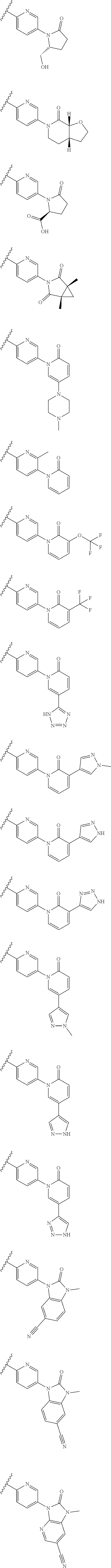

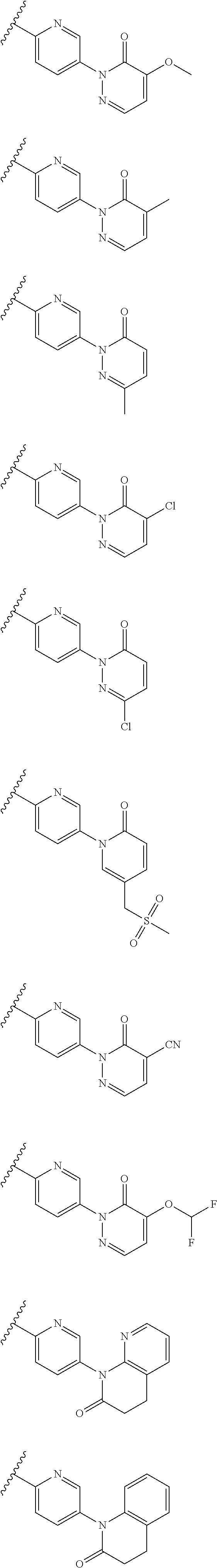

19. The compound of claim 1 or a pharmaceutically acceptable salt thereof, wherein A-B—C is of the formula (I-A), (I-B), (I-Ba), (I-Bb), (I-C), or (I-D): 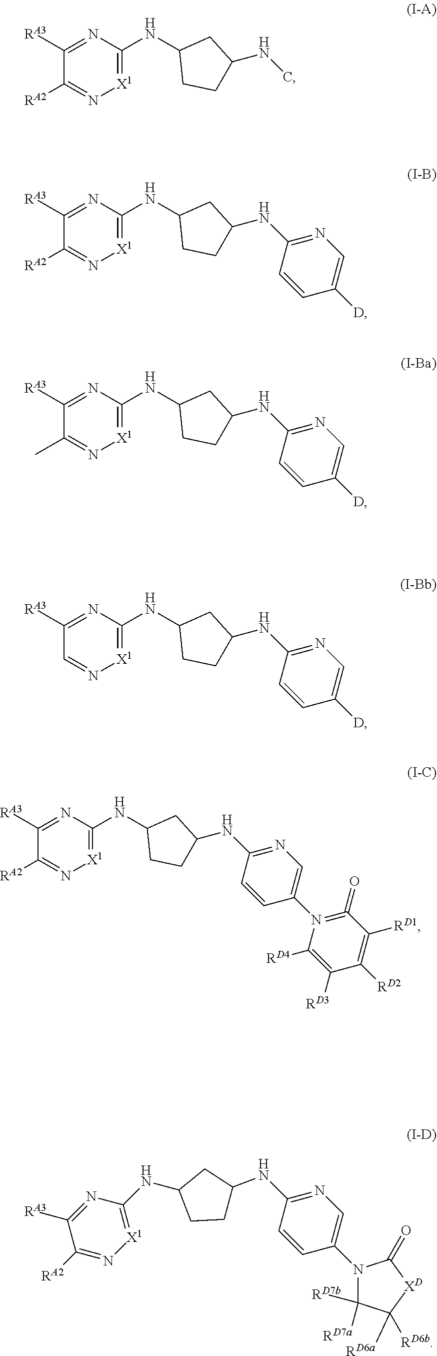
wherein X¹, R^{A2}, R^{A3}, C, D, R^{D1}, R^{D2}, R^{D3}, R^{D4}, R^{D1a}, R^{D2a}, R^{D3a}, R^{D4a}, X^{D}, R^{D6a}, R^{D6b}, R^{D7a} and R^{D7b} are as defined in any of the preceding claims.

20. The compound of claim 1 which is: 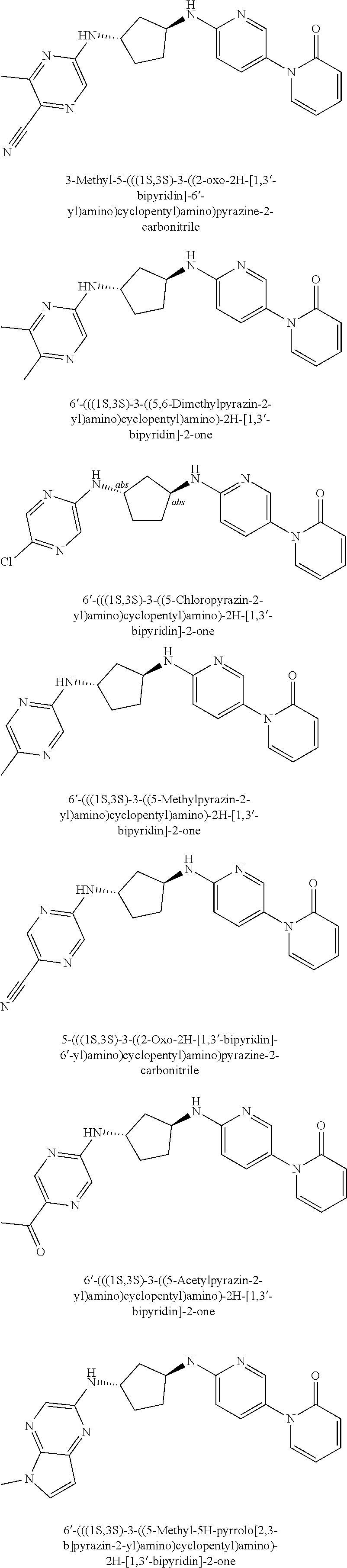 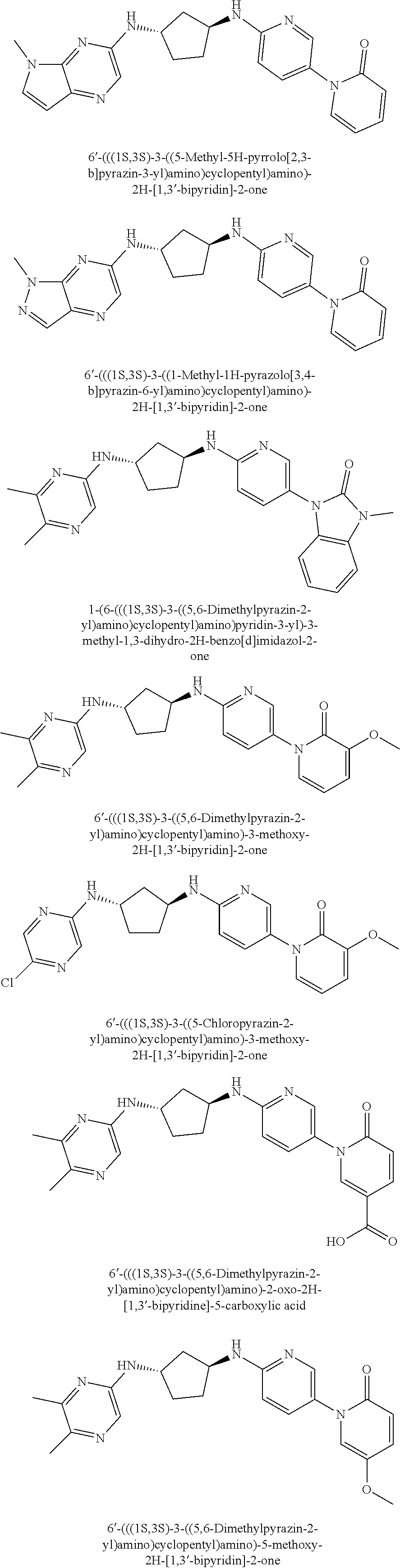 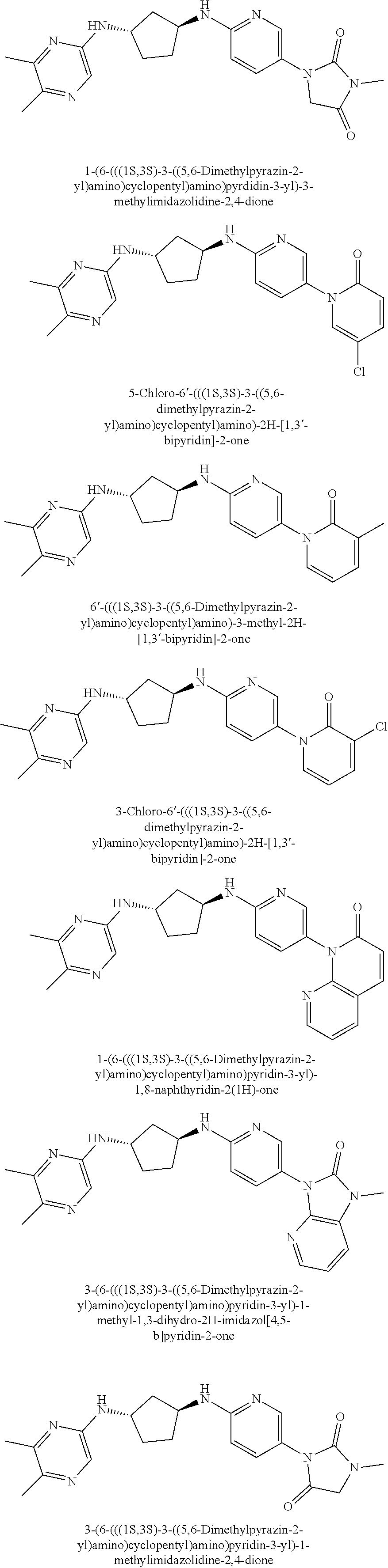 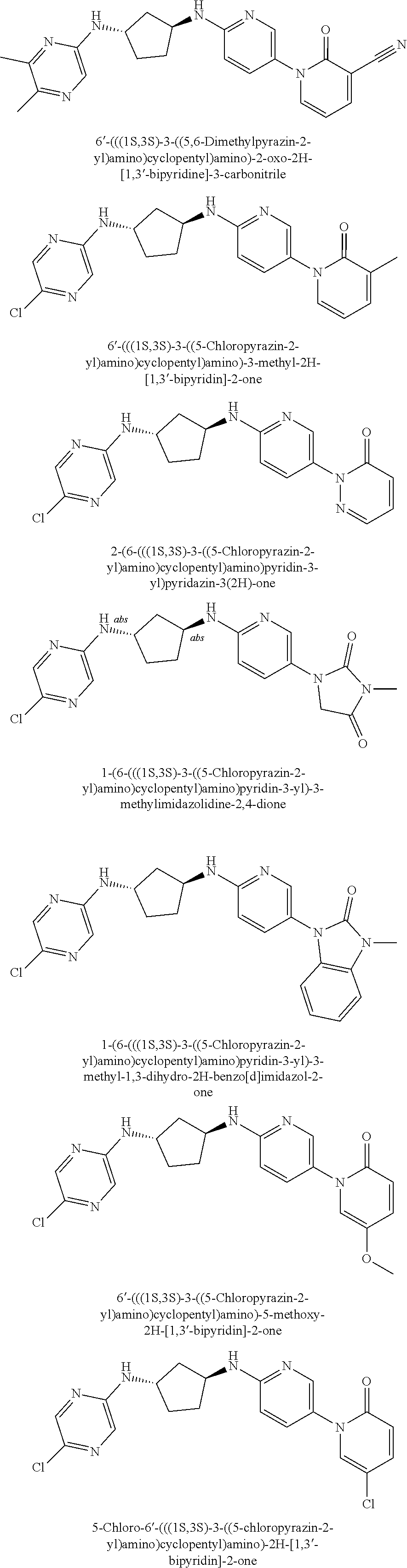 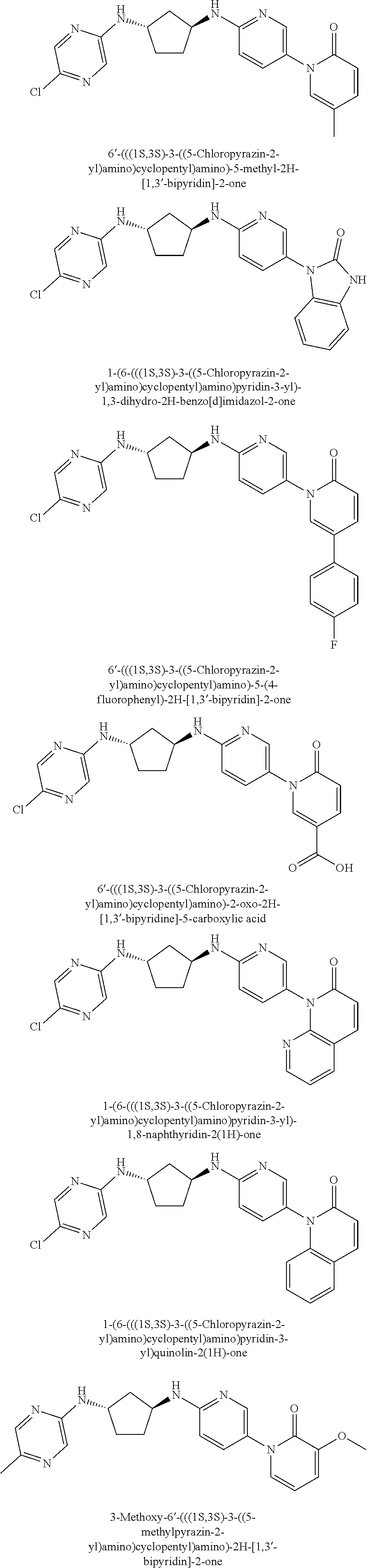 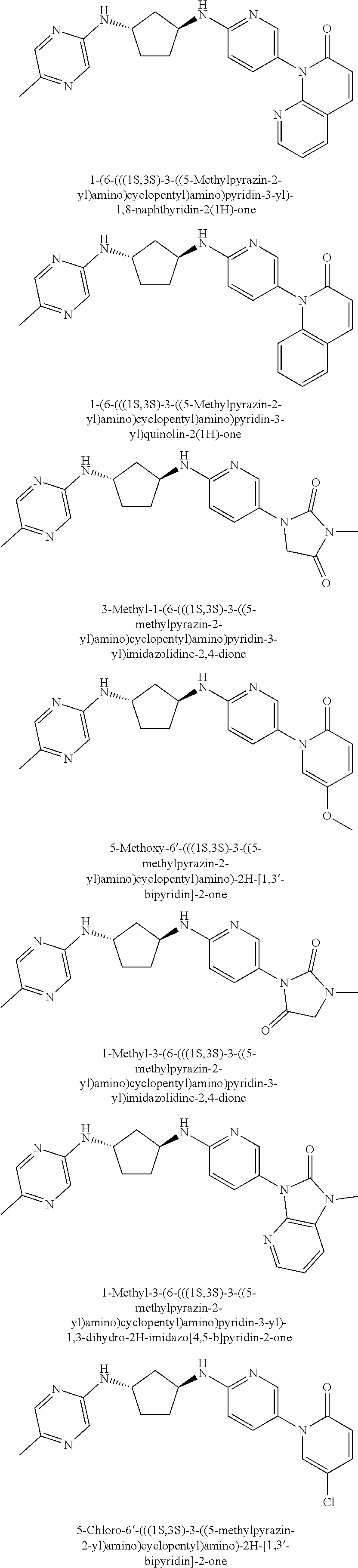 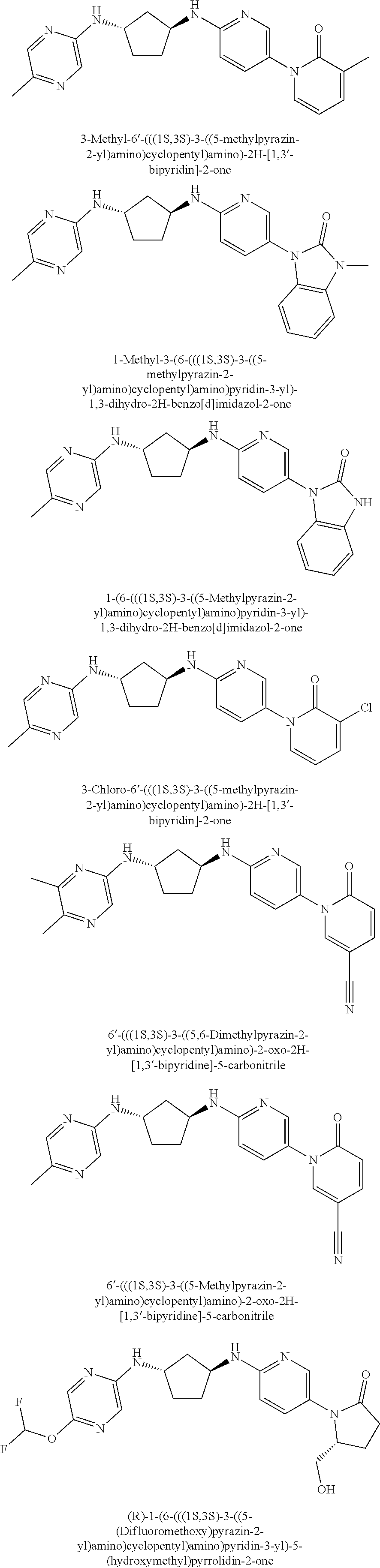 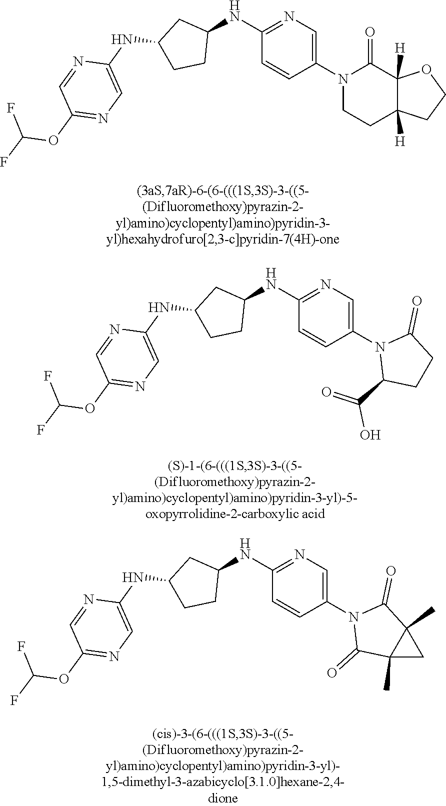 [SMILES: O=C1[C@@H]2OCC[C@@H]2CCN1c1ccc(N[C@H]2CC[C@H](Nc3cnc(OC(F)F)cn3)C2)nc1 ; O=C(O)[C@@H]1CCC(=O)N1c1ccc(N[C@H]2CC[C@H](Nc3cnc(OC(F)F)cn3)C2)nc1 ; C[C@@]12C[C@]1(C)C(=O)N(c1ccc(N[C@H]3CC[C@H](Nc4cnc(OC(F)F)cn4)C3)nc1)C2=O] 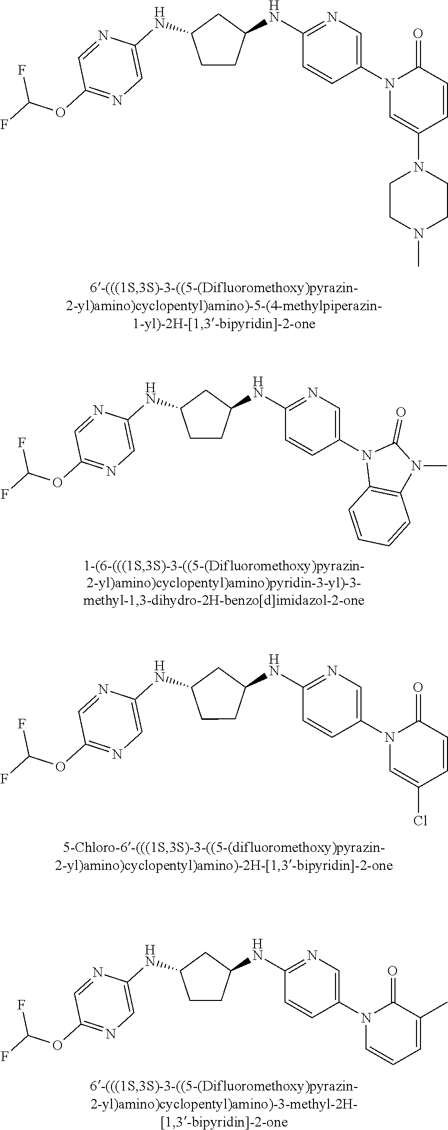 [SMILES: CN1CCN(c2ccc(=O)n(-c3ccc(N[C@H]4CC[C@H](Nc5cnc(OC(F)F)cn5)C4)nc3)c2)CC1 ; Cn1c(=O)n(-c2ccc(N[C@H]3CC[C@H](Nc4cnc(OC(F)F)cn4)C3)nc2)c2ccccc21 ; O=c1ccc(Cl)cn1-c1ccc(N[C@H]2CC[C@H](Nc3cnc(OC(F)F)cn3)C2)nc1 ; Cc1cccn(-c2ccc(N[C@H]3CC[C@H](Nc4cnc(OC(F)F)cn4)C3)nc2)c1=O] 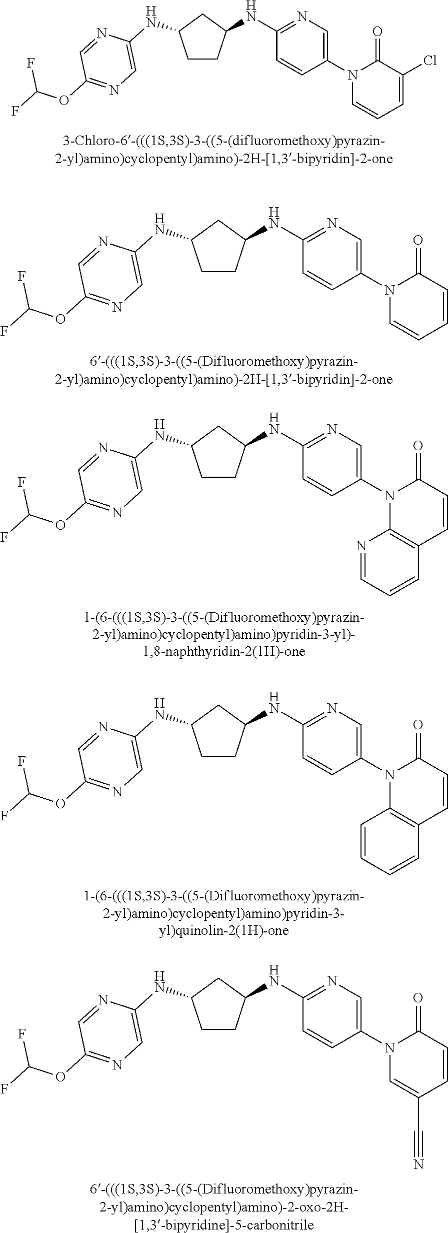 [SMILES: O=c1c(Cl)cccn1-c1ccc(N[C@H]2CC[C@H](Nc3cnc(OC(F)F)cn3)C2)nc1 ; O=c1ccccn1-c1ccc(N[C@H]2CC[C@H](Nc3cnc(OC(F)F)cn3)C2)nc1 ; O=c1ccc2cccnc2n1-c1ccc(N[C@H]2CC[C@H](Nc3cnc(OC(F)F)cn3)C2)nc1 ; O=c1ccc2ccccc2n1-c1ccc(N[C@H]2CC[C@H](Nc3cnc(OC(F)F)cn3)C2)nc1 ; N#Cc1ccc(=O)n(-c2ccc(N[C@H]3CC[C@H](Nc4cnc(OC(F)F)cn4)C3)nc2)c1] 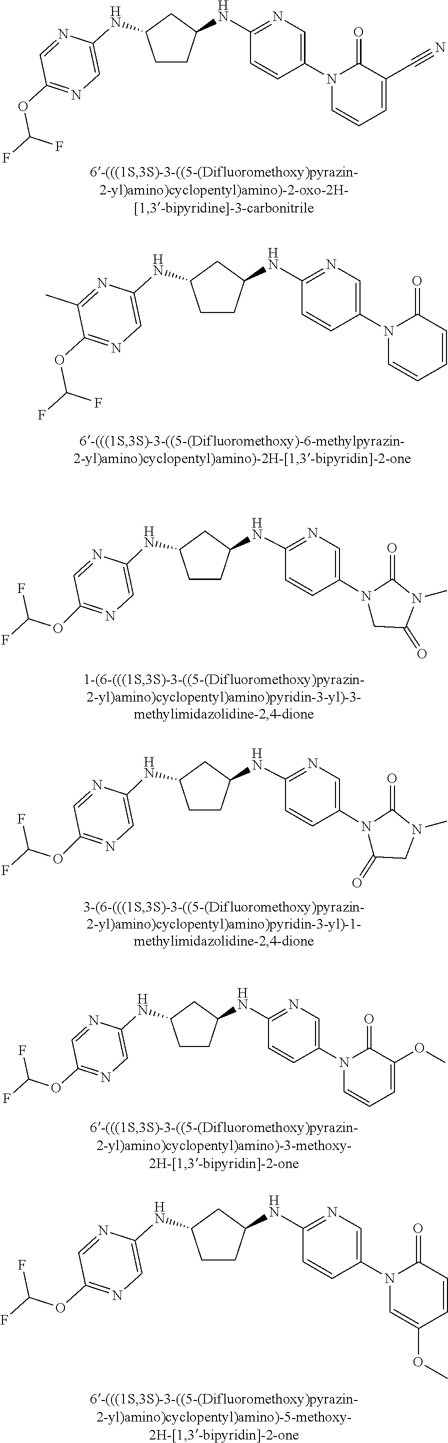 [SMILES: N#Cc1cccn(-c2ccc(N[C@H]3CC[C@H](Nc4cnc(OC(F)F)cn4)C3)nc2)c1=O ; Cc1nc(N[C@H]2CC[C@H](Nc3ccc(-n4ccccc4=O)cn3)C2)cnc1OC(F)F ; CN1C(=O)CN(c2ccc(N[C@H]3CC[C@H](Nc4cnc(OC(F)F)cn4)C3)nc2)C1=O ; CN1CC(=O)N(c2ccc(N[C@H]3CC[C@H](Nc4cnc(OC(F)F)cn4)C3)nc2)C1=O ; COc1cccn(-c2ccc(N[C@H]3CC[C@H](Nc4cnc(OC(F)F)cn4)C3)nc2)c1=O ; COc1ccc(=O)n(-c2ccc(N[C@H]3CC[C@H](Nc4cnc(OC(F)F)cn4)C3)nc2)c1] 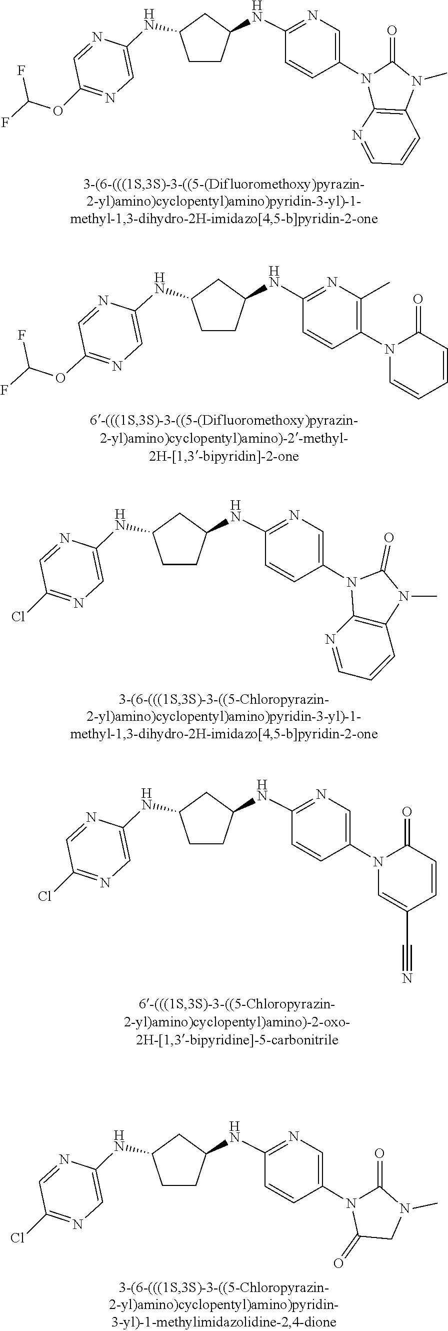 [SMILES: Cn1c(=O)n(-c2ccc(N[C@H]3CC[C@H](Nc4cnc(OC(F)F)cn4)C3)nc2)c2ncccc21 ; Cc1nc(N[C@H]2CC[C@H](Nc3cnc(OC(F)F)cn3)C2)ccc1-n1ccccc1=O ; Cn1c(=O)n(-c2ccc(N[C@H]3CC[C@H](Nc4cnc(Cl)cn4)C3)nc2)c2ncccc21 ; N#Cc1ccc(=O)n(-c2ccc(N[C@H]3CC[C@H](Nc4cnc(Cl)cn4)C3)nc2)c1 ; CN1CC(=O)N(c2ccc(N[C@H]3CC[C@H](Nc4cnc(Cl)cn4)C3)nc2)C1=O] 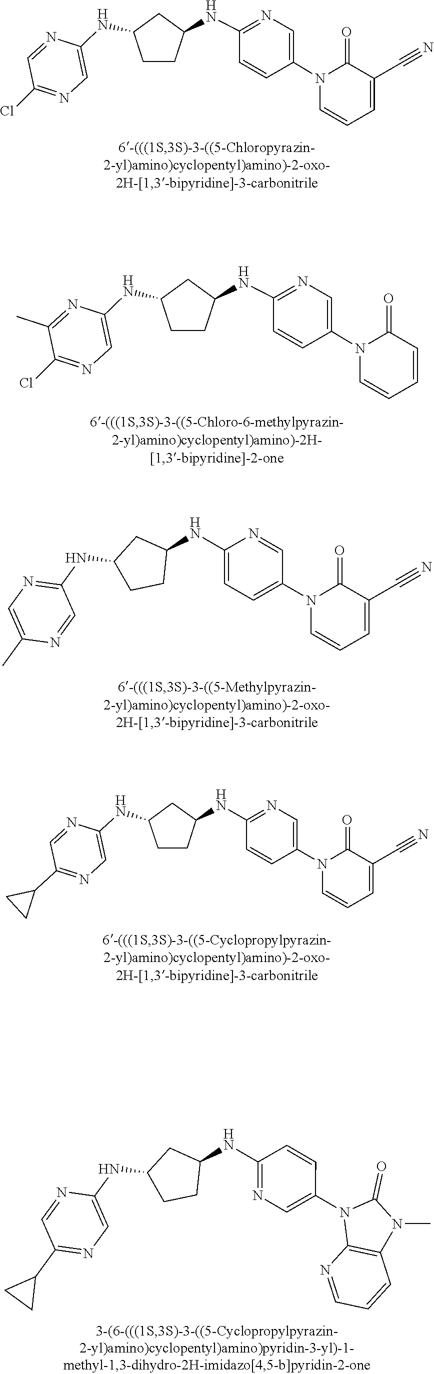 [SMILES: N#Cc1cccn(-c2ccc(N[C@H]3CC[C@H](Nc4cnc(Cl)cn4)C3)nc2)c1=O ; Cc1nc(N[C@H]2CC[C@H](Nc3ccc(-n4ccccc4=O)cn3)C2)cnc1Cl ; Cc1cnc(N[C@H]2CC[C@H](Nc3ccc(-n4cccc(C#N)c4=O)cn3)C2)cn1 ; N#Cc1cccn(-c2ccc(N[C@H]3CC[C@H](Nc4cnc(C5CC5)cn4)C3)nc2)c1=O ; Cn1c(=O)n(-c2ccc(N[C@H]3CC[C@H](Nc4cnc(C5CC5)cn4)C3)nc2)c2ncccc21] 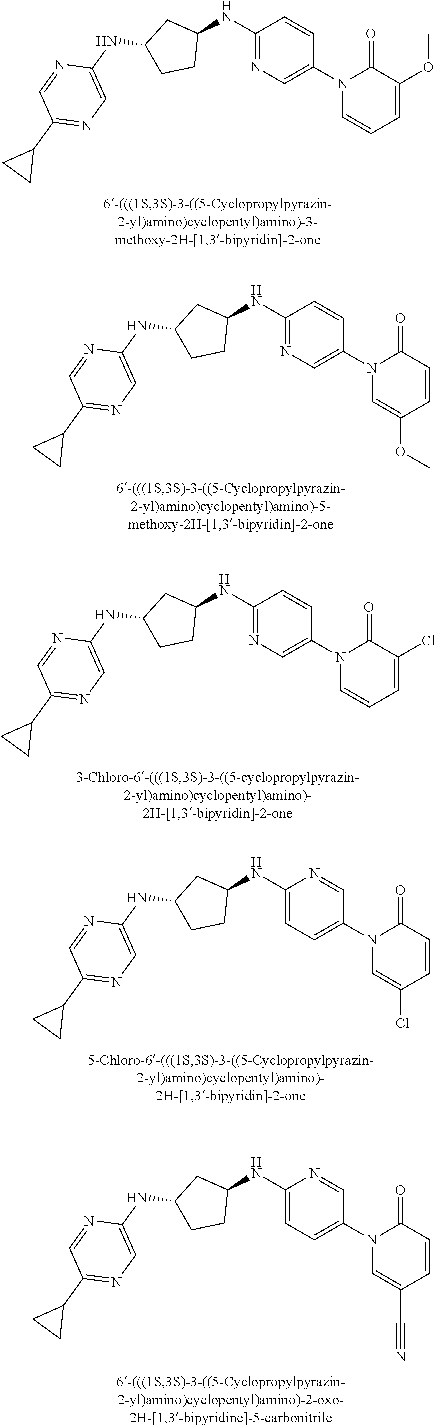 [SMILES: COc1cccn(-c2ccc(N[C@H]3CC[C@H](Nc4cnc(C5CC5)cn4)C3)nc2)c1=O ; COc1ccc(=O)n(-c2ccc(N[C@H]3CC[C@H](Nc4cnc(C5CC5)cn4)C3)nc2)c1 ; O=c1c(Cl)cccn1-c1ccc(N[C@H]2CC[C@H](Nc3cnc(C4CC4)cn3)C2)nc1 ; O=c1ccc(Cl)cn1-c1ccc(N[C@H]2CC[C@H](Nc3cnc(C4CC4)cn3)C2)nc1 ; N#Cc1ccc(=O)n(-c2ccc(N[C@H]3CC[C@H](Nc4cnc(C5CC5)cn4)C3)nc2)c1] 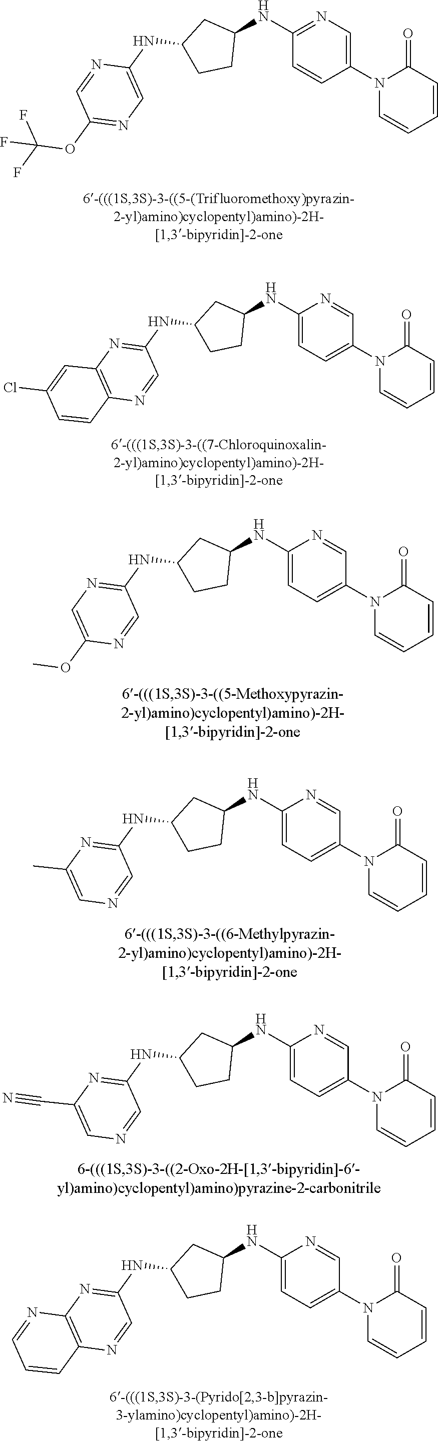 [SMILES: O=c1ccccn1-c1ccc(N[C@H]2CC[C@H](Nc3cnc(OC(F)(F)F)cn3)C2)nc1 ; O=c1ccccn1-c1ccc(N[C@H]2CC[C@H](Nc3cnc4ccc(Cl)cc4n3)C2)nc1 ; COc1cnc(N[C@H]2CC[C@H](Nc3ccc(-n4ccccc4=O)cn3)C2)cn1 ; Cc1cncc(N[C@H]2CC[C@H](Nc3ccc(-n4ccccc4=O)cn3)C2)n1 ; N#Cc1cncc(N[C@H]2CC[C@H](Nc3ccc(-n4ccccc4=O)cn3)C2)n1 ; O=c1ccccn1-c1ccc(N[C@H]2CC[C@H](Nc3cnc4cccnc4n3)C2)nc1] 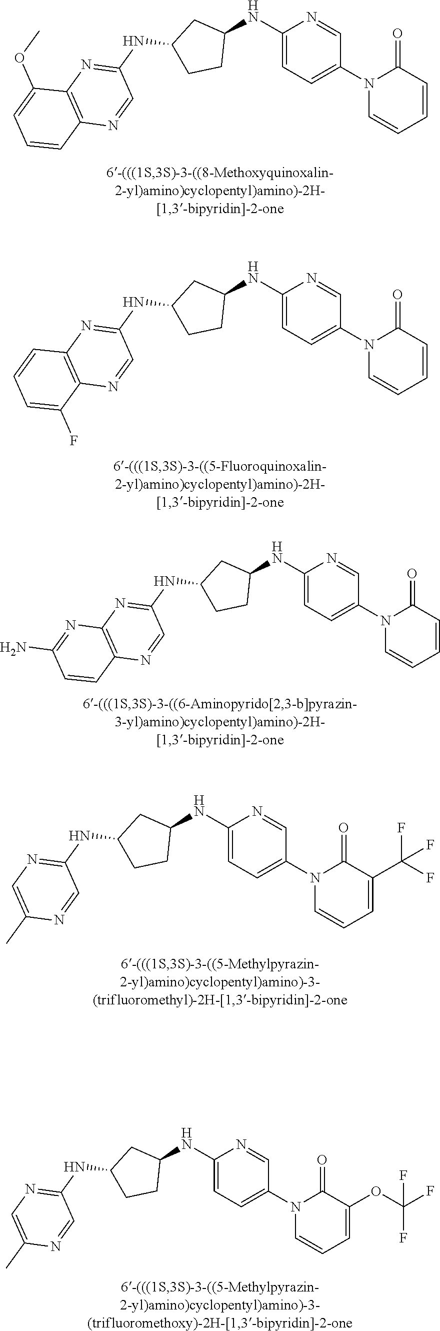 [SMILES: COc1cccc2ncc(N[C@H]3CC[C@H](Nc4ccc(-n5ccccc5=O)cn4)C3)nc12 ; O=c1ccccn1-c1ccc(N[C@H]2CC[C@H](Nc3cnc4c(F)cccc4n3)C2)nc1 ; Nc1ccc2ncc(N[C@H]3CC[C@H](Nc4ccc(-n5ccccc5=O)cn4)C3)nc2n1 ; Cc1cnc(N[C@H]2CC[C@H](Nc3ccc(-n4cccc(C(F)(F)F)c4=O)cn3)C2)cn1 ; Cc1cnc(N[C@H]2CC[C@H](Nc3ccc(-n4cccc(OC(F)(F)F)c4=O)cn3)C2)cn1]
or a pharmaceutically acceptable salt thereof.

21. (canceled)

22. A pharmaceutical composition comprising the compound of claim 1 or a pharmaceutically acceptable salt thereof, and a pharmaceutically acceptable diluent, carrier or excipient.

23. (canceled)

24. (canceled)

25. A method of treating PCSK9-mediated disease or disorder in a patient need thereof comprising administering to the patient a therapeutically effective amount of the compound or pharmaceutically acceptable salt thereof of claim 1.

26. The method according to claim 25, wherein the disease or disorder is a cardiovascular disease or disorder.

27. The method of claim 26 wherein the cardiovascular disease is selected from dyslipidemia, hypercholesterolemia, hypertriglyceridemia, hyperlipidemia, hypoalphalipoproteinemia, metabolic syndrome, diabetic complications, atherosclerosis, stroke, vascular dimensia, chronic kidney disease, coronary heart disease, coronary artery disease, retinopathy, inflammation, thrombosis, peripheral vascular disease heart failure or congestive heart failure.

28. The method of claim 27 wherein the compound is administered simultaneously, separately or sequentially in combination with an additional active ingredient selected from the group consisting of:
i) a statin;
ii) a cholesterol absorption inhibitor;
iii) a SGLT2 inhibitor;
iv) a P2Y12 inhibitor;
v) a citrate lyase inhibitor; and
vi) anti-hypertensive drugs.


SEQUENCE LISTING (1 sequence)

SEQ ID NO: 1 - protein, 705 aa, synthetic construct
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQENLYFQGHHHHHH